(19) United States
(12) Patent Application Publication
(10) Pub. No.: US 2014/0135359 A1
(43) Pub. Date: May 15, 2014

(54) METHODS OF DESIGNING, PREPARING, AND USING NOVEL PROTONOPHORES

(71) Applicant: Louis C. Martineau, Saint-Laurent (CA)

(72) Inventor: Louis C. Martineau, Saint-Laurent (CA)

(21) Appl. No.: 13/674,211

(22) Filed: Nov. 12, 2012

(51) Int. Cl. A01N 43/80; A01N 35/02; A61K 31/095; A01N 41/12; A61K 31/352; A01N 43/16; A61K 31/122; A01N 35/04; A61K 31/351; A61K 31/382; A01N 43/18; A61K 31/341; A01N 43/08; A61K 31/421; A01N 43/76; A61K 31/42; A61K 31/44; A01N 43/40; A61K 31/47; A01N 43/42; A61K 31/4745; A61K 31/12

(52) U.S. Cl. CPC A01N 43/80; A61K 31/12; A01N 35/02; A61K 31/095; A01N 41/12; A61K 31/352; A01N 43/16; A61K 31/122; A01N 35/04; A61K 31/351; A61K 31/382; A01N 43/18; A61K 31/341; A01N 43/08; A61K 31/421; A01N 43/76; A61K 31/42; A61K 31/44; A01N 43/40; A61K 31/47; A01N 43/42; A61K 31/4745
USPC 514/292; 436/138; 435/29; 514/678; 514/706; 514/456; 514/682; 514/680; 514/460; 514/432; 514/473; 514/376; 514/380; 514/352; 514/313

(57) ABSTRACT

The present invention provides a computer-assisted method of generating a protonophore requiring the use of a computer including a processor. The method includes: designing the protonophore, calculating, using the processor, an estimated protonophoric activity; producing the protonophore if the estimated protonophoric activity corresponds to an U₅₀ of about 20 μM or less; and determining the uncoupling activity of the protonophore. The present invention also provides novel protonophores that meet the above requirement and their methods of use.

### BACKGROUND OF THE INVENTION

Protonophores are small xenobiotic chemical compounds that can specifically facilitate the diffusion of protons across a biological membrane characterized by a proton gradient when such compounds exist in both unionized and ionized forms on both sides of said biological membrane. By facilitating the diffusion of protons, protonophores can uncouple oxidative phosphorylation, thereby reducing metabolic efficiency and perturbing energy homeostasis. As such, protonophores have historically been used for the control of pests. However, the effect of protonophores on energy transduction can have the benefit of activating the AMP-activated protein kinase signaling pathway, an important therapeutic target for insulin resistance and related conditions.

Currently, insulin resistance and related conditions are treated with metformin, a compound that can also activate the AMP-activated protein kinase signaling pathway. However, metformin has limited efficacy and low potency, and better alternatives are currently being sought. The mechanism through which protonophores act to activate the AMP-activated protein kinase signaling pathway can be more effective than that of metformin, especially in skeletal muscle, a key target for glucose homeostasis. A protonophore-based treatment may therefore represent a better alternative to metformin, especially if the protonophore is designed without inclusion of the metabolically-stable chemical groups that characterize protonophores used for the control of pests. Moreover, protonophores with low bioavailability may be useful antimicrobial agents. Finally, protonophores with low environmental persistance may be a useful alternative to products currently used for the control of pests.

### SUMMARY OF THE INVENTION

The present invention provides a computer-assisted method of generating a protonophore, the method requiring the use of a computer including a processor. The method includes: designing the protonophore; calculating, using the processor, an estimated protonophoric activity across a biological membrane with a pH gradient for the protonophore; producing the protonophore if the estimated protonophoric activity across the biological membrane with the pH gradient for the protonophore corresponds to an U₅₀ of about 20 μM or less; and determining the uncoupling activity of the protonophore. The present invention also provides novel protonophores that meet the above requirement and their methods of use.

In one embodiment, the biological membrane with the pH gradient includes an inner membrane of a mitochondrion, a thylakoid membrane of a chloroplast, an outer membrane of an aerobic bacterium, or an outer membrane of an archaeum.

In one embodiment, the designing the protonophore includes: adding one or more hydroxyl or thiol groups to an aromatic or a heteroaromatic ring system or replacing one or more of ring atoms of the aromatic or heteroaromatic ring system with one or more unsubstituted acidic or basic nitrogen atoms to provide a first ionizable intermediate having a proportion of an unionized species and a proportion of an ionized species on a first and on a second side of a biological membrane, wherein the aromatic or the heteroaromatic ring system is unsubstituted or substituted with one or more oxygen atoms; provided that if the proportion of the ionized species is less than about one thousand times greater than the proportion of the unionized species on either the first side or the second side of the biological membrane or that the proportion of the unionized species is less than about one thousand times greater than the proportion of the ionized species on either the first side or the second side of the biological membrane, then adding one or more acidity-modulating substituents directly to the aromatic or the heteroaromatic ring system of the first ionizable intermediate to provide a second ionizable intermediate having the proportion of the ionized species two or more times greater than the proportion of the unionized species on both the first and the second sides of the biological membrane or having the proportion of the unionized species two or more times greater than the proportion of the ionized species on both the first and the second sides of the biological membrane, provided that the one or more acidity-modulating substituents do not include one or more nitro groups or one or more cyano groups; and adding one or more lipophilicity-conferring substituents directly to the aromatic or the heteroaromatic ring system of the first ionizable intermediate or the second ionizable intermediate or to the one or more acidity-modulating substituents of the second ionizable intermediate to provide the protonophore, wherein the protonophore exhibits a planar and a linear three-dimensional geometry, and provided that: if the proportion of the unionized species of the protonophore is greater than the proportion of the ionized species of the protonophore, then the ionized species exhibits a greater degree of diffusibility across a biological membrane than the unionized species, or if the proportion of the ionized species of the protonophore is greater than the proportion of the unionized species of the protonophore, then the unionized species exhibits a greater degree of diffusibility across the biological membrane than the ionized species.

In one embodiment, the one or more acidity-modulating substituents each independently include formyl or NH₂. In one embodiment, the one or more lipophilicity-conferring substituents each independently include (C₁-C₁₂)alkyl, (C₁-C₁₂)alkenyl, (C₁-C₁₂)aldehyde, (C₁-C₁₂)alkoxy, (C₆-C₁₂)aryl, halogen, or haloalkyl. In one embodiment, the one or more lipophilicity-conferring substituents each independently include (C₁-C₈)alkyl, (C₁-C₈)alkenyl, (C₁-C₆)aldehyde, or (C₁-C₆)alkoxy.

In one embodiment, the calculating, using the processor, an estimated protonophoric activity across a biological membrane with a pH gradient for the protonophore includes: calculating, using the processor, the estimated protonophoric activity across the biological membrane with the pH gradient for the protonophore as a function of an inverse of a first sum of a first resistance to diffusion across the biological membrane with the pH gradient for an unionized species of the protonophore and of a second resistance to diffusion across the biological membrane with the pH gradient for an ionized species of the protonophore; and comparing the estimated protonophoric activity across the biological membrane with the pH gradient for the protonophore with a second estimated protonophoric activity for a reference protonophore of known U₅₀.

In one embodiment, the determining the first resistance to diffusion across the biological membrane with the pH gradient for the unionized species of the protonophore and determining the second resistance to diffusion across the biological membrane with the pH gradient for the unionized species of the protonophore includes: calculating the first resistance to diffusion across the biological membrane with the pH gradient for the unionized species of the protonophore as an inverse function of a first permeability across the biological membrane with the pH gradient for the unionized species of the protonophore and a function of a first ratio of a first number of molecules of the unionized species of the protonophore at a steady-state condition on a first side of the biological membrane with the pH gradient from which the unionized species of the protonophore translocates over a second number of molecules of the ionized species of the protonophore at the steady-state condition on a second opposite side of the biological membrane with the pH gradient; and calculating the second resistance to diffusion across the biological membrane with the pH gradient for the ionized species of the protonophore as an inverse function of a second permeability across the biological membrane with the pH gradient for the ionized species of the protonophore and a function of the first ratio.

In one embodiment, the determining the uncoupling activity of the protonophore includes measuring an increase of a rate of oxygen consumption in a preparation of isolated mitochondria, in a preparation of cells in culture, or in a preparation of tissues in culture, or measuring a bactericidal or a bacteriostatic effect, a fungicidal or a fungistatic effect, a herbicidal effect, or a pesticidal effect.

In one embodiment, the protonophore includes a compound of Formula (I)

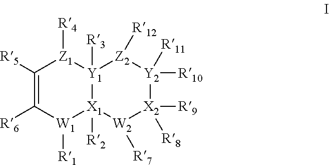

wherein: W₁ is carbon, oxygen, sulfur, or nitrogen; X₁, Y₁, and Z₁ are each independently carbon;

R′₁ is absent, hydroxyl, or thiol; R′₂ is hydrogen, (C1-C6)aldehyde, (C1-C8)alkyl, acetyl, amino, (C1-C6)dialkylamino, or (C1-C8)alkenyl; R′₃ is hydrogen, hydroxyl, thiol, (C1-C6)aldehyde, (C1-C8)alkyl, or (C1-C8)alkenyl; R′₄ is hydrogen, (C1-C6)aldehyde, hydroxyl, (C1-C8)alkyl, (C1-C6)dialkylamino, (C1-C8)alkyl, acetyl, (C1-C8)alkenyl, CO(C1-C8)alkenyl, CO(C1-C8)alkyl, or CO-p-C₆H₅SH; R′₅ is hydrogen, (C1-C6)aldehyde, (C1-C8)alkyl, thiol, acetyl, (C1-C8)alkenyl, or CO(C1-C8)alkenyl; and R′₆ is hydrogen, amino, (C1-C6)dialkylamino, (C1-C6)aldehyde, (C1-C8)alkyl, (C1-C6)alkoxy, acetyl, (C1-C8)alkenyl, CO(C1-C8)alkenyl, or CO(C1-C8)alkyl, provided that W₂, R′₇, X₂, R′₈, R′₉, Y₂, R′₁₀, R′₁₁, Z₂, and R′₁₂ are absent; or

wherein: W₁, X₁, Y₁, and Z₁ are each independently carbon; W₂ is oxygen, carbon, or nitrogen; X₂ is carbon or nitrogen; Y₂ and Z₂ are each independently carbon; R′₁ is hydrogen, (C1-C6)aldehyde, (C1-C8)alkyl, acetyl, (C1-C8)alkenyl, CO(C1-C8)alkenyl, or CO(C1-C8)alkyl; R′₂ and R′₃ are each independently absent; R′₄ is hydrogen, (C1-C6)aldehyde, (C1-C8)alkyl, acetyl, or (C1-C8)alkenyl; R′₅ is hydrogen, hydroxyl, thiol, amino, (C1-C8)alkyl, (C1-C6)aldehyde, acetyl, (C1-C8)alkylamino, (C1-C8)alkenyl, CO(C1-C8)alkenyl, or CO(C1-C8)alkyl; R′₆ is hydrogen, hydroxyl, (C1-C6)aldehyde, thiol, (C1-C8)alkyl, amino, (C1-C8)alkylamino, acetyl, (C1-C8)alkenyl, CO(C1-C8)alkenyl, or CO(C1-C8)alkyl; R′₇ is absent, hydrogen, (C1-C6)aldehyde, amino, or (C1-C8)alkylamino; R′₈ is absent, hydrogen, acetyl, (C1-C8)alkyl, amino, (C1-C8)alkylamino, (C1-C8)alkenyl, or CO(C1-C8)alkenyl or R′₈, R′₉, R′₁₀ and R′₁₁ together form two carbon atoms of an unsubstituted or substituted(C₁-C₁₂)aryl or two carbon atoms of an unsubstituted or substituted(C₁-C₁₂)heteroaromatic; R′₉ and R′₁₁ are each independently absent or hydrogen; R′₁₀ is hydrogen, hydroxyl, thiol, amino, (C1-C8)alkylamino, (C1-C8)alkyl, or (C1-C8)alkenyl; R′₁₂ is carbonyl, hydrogen, amino, (C1-C8)alkylamino, or (C1-C8)alkyl; or

wherein: W₁ is oxygen, sulfur, carbon, or nitrogen; X₁ and Y₁ are each independently carbon or nitrogen; Z₁ is absent; W₂ and Z₂ are each independently hydrogen; R′₁ is absent, hydrogen, or carbonyl; R′₂ is absent, hydrogen, thiol, hydroxyl, (C1-C8)alkyl, acetyl, (C1-C8)alkenyl, CO(C1-C8)alkenyl, CO(C1-C8)alkyl; or CO—(C1-C6)aryl; R′₃ is absent, hydrogen, thiol, hydroxyl, (C1-C6)aldehyde, (C1-C8)alkyl, acetyl, CO(C1-C8)alkenyl, or CO(C1-C8)alkyl; R′₄ is absent; R′₅ is hydrogen, (C1-C8)alkyl, (C1-C8)alkenyl, or CO(C1-C8)alkenyl; or R′₅ and R′₆ together form two carbon atoms of an unsubstituted or substituted(C₁-C₁₂)aryl; R′₆ is hydroxyl, (C1-C6)aldehyde, acetyl, (C1-C8)alkyl, (C1-C8)alkenyl, CO(C1-C8)alkenyl, or CO(C1-C8)alkyl; W₂, X₂, Y₂, Z₂, R′₇, R′₈, R′₉, R′₁₀, R′₁₁, and R′₁₂ is absent; and R′₂, R′₃, Z₁, W₂, X₂, Y₂, Z₂, R′₇, R′₈, R′₉, R′₁₀, R′₁₁, and R′₁₂ together form two carbon atoms of an unsubstituted or substituted(C₁-C₁₂)aryl; or

wherein: W₁ is oxygen; X₁, Y₁, and Z₁ are each independently carbon; R′₁, R′₂, and R′₃ are each independently absent; R′₄ and R′₆ are each independently (C1-C8)alkyl; R′₅ is hydroxyl; W₂ and Z₂ are each independently CH; X₂ and Y₂ are each independently carbon; R′₇ and R′₁₂ are each independently hydrogen; and R′₈, R′₉, R′₁₀ and R′₁₁ together form two carbon atoms of an unsubstituted or substituted(C1-C12)heteroaromatic, or a pharmaceutically acceptable salt, solvate, or prodrug thereof.

In one embodiment, the protonophore is a mono-protic protonophore or a multi-protic protonophore.

The present invention provides a method of designing a protonophore. The method includes: adding one or more hydroxyl or thiol groups to an aromatic or a heteroaromatic ring system or replacing one or more of ring atoms of the aromatic or heteroaromatic ring system with one or more unsubstituted acidic or basic nitrogen atoms to provide a first ionizable intermediate having a proportion of an unionized species and a proportion of an ionized species on a first and on a second side of a biological membrane, wherein the aromatic or the heteroaromatic ring system is unsubstituted or substituted with one or more oxygen atoms; provided that if the proportion of the ionized species is less than about one thousand times greater than the proportion of the unionized species on either the first side or the second side of the biological membrane or that the proportion of the unionized species is less than about one thousand times greater than the proportion of the ionized species on either the first side or the second side of the biological membrane, then adding one or more acidity-modulating substituents directly to the aromatic or the heteroaromatic ring system of the first ionizable intermediate to provide a second ionizable intermediate having the proportion of the ionized species two or more times greater than the proportion of the unionized species on both the first and the second sides of the biological membrane or having the proportion of the unionized species two or more times greater than the proportion of the ionized species on both the first and the second sides of the biological membrane, provided that the one or more acidity-modulating substituents do not include one or more nitro groups or one or more cyano groups; and adding one or more lipophilicity-conferring substituents directly to the aromatic or the heteroaromatic ring system of the first ionizable intermediate or the second ionizable intermediate or to the one or more acidity-modulating substituents of the second ionizable intermediate to provide the protonophore, wherein the protonophore exhibits a planar and a linear three-dimensional geometry, and provided that: if the proportion of the unionized species of the protonophore is greater than the proportion of the ionized species of the protonophore, then the ionized species exhibits a greater degree of diffusibility across a biological membrane than the unionized species, or if the proportion of the ionized species of the protonophore is greater than the proportion of the unionized species of the protonophore, then the unionized species exhibits a greater degree of diffusibility across the biological membrane than the ionized species.

In one embodiment, the one or more heteroatoms of the heteroaromatic ring system each independently include nitrogen, oxygen, sulfur, or a combination thereof. In one embodiment, the aromatic or the heteroaromatic ring system includes a fused or unfused (C₆-C₃₀)aromatic ring system or a fused or unfused (C₁-C₃₀)heteroaromatic ring system. In one embodiment, the aromatic or the heteroaromatic ring system includes a fused (C₆-C₃₀)aromatic ring system. In one embodiment, the aromatic or the heteroaromatic ring system includes an unfused (C₆-C₃₀)aromatic ring system. In one embodiment, the aromatic or the heteroaromatic ring system includes a fused (C₁-C₃₀)heteroaromatic ring system. In one embodiment, the aromatic or the heteroaromatic ring system includes an unfused (C₁-C₃₀)heteroaromatic ring system. In one embodiment, the one or more acidity-modulating substituents each independently include formyl or NH₂. In one embodiment, the one or more lipophilicity-conferring substituents each independently include (C₁-C₁₂)alkyl, (C₁-C₁₂)alkenyl, (C₁-C₁₂)aldehyde, (C₁-C₁₂)alkoxy, (C₆-C₁₂)aryl, halogen, or haloalkyl. In one embodiment, the one or more lipophilicity-conferring substituents each independently include (C₁-C₃)alkyl, (C₁-C₃)alkenyl, (C₁-C₆)aldehyde, or (C₁-C₆)alkoxy.

In one embodiment, a degree of lipophilicity for diffusibility across the biological membrane is from about 2.8 to about 4.0 log P (_{octanol-water}). In one embodiment, the degree of lipophilicity for diffusibility across the biological membrane is from about 3.0 to about 3.6 log P (_{octanol-water}). In one embodiment, the biological membrane includes an inner membrane of a mitochondrion, a thylakoid membrane of a chloroplast, an outer membrane of an aerobic bacterium, or an outer membrane of an archaeum. In one embodiment, the protonophore is a mono-protic protonophore or a multi-protic protonophore. In one embodiment, the mono-protic protonophore has a pKₐ from about 4.0 to about 7.0 and from about 8.4 to about 11.4. In one embodiment, the mono-protic protonophore has a pKₐ from about 4.0 to about 6.0 and from about 9.4 to about 11.4. In one embodiment, the mono-protic protonophore has a pKₐ within about 4 pH units below a first pH of a high pH compartment and about 4 pH units above a second pH of a low pH compartment, wherein the high pH compartment and the low pH compartment are separated by the biological membrane. In one embodiment, the multi-protic protonophore has the unionized species and at least one ionized species present at the first pH the high pH compartment and the second pH of the low pH compartment, and the unionized species or one of the ionized species is predominant at these pH values. In one embodiment, the protonophore has a log P (_{octanol-water}) from about 2.8 to about 6.0. In one embodiment, the protonophore has a log P (_{octanol-water}) of about 3.0 to about 5.5.

The present invention provides a computer-assisted method of calculating an estimated protonophoric activity across a biological membrane with a pH gradient for a protonophore, the method requiring the use of a programmed computer including a processor. The method includes: calculating, using the processor, the estimated protonophoric activity across the biological membrane with the pH gradient for the protonophore as a function of an inverse of a first sum of a first resistance to diffusion across the biological membrane with the pH gradient for an unionized species of the protonophore and of a second resistance to diffusion across the biological membrane with the pH gradient for an ionized species of the protonophore; and comparing the estimated protonophoric activity across the biological membrane with the pH gradient for the protonophore with a second estimated protonophoric activity for a reference protonophore of known U₅₀.

In one embodiment, the biological membrane with the pH gradient includes an inner membrane of a mitochondrion, a thylakoid membrane of a chloroplast, an outer membrane of an aerobic bacterium, or an outer membrane of an archaeum. In one embodiment, the biological membrane is an inner membrane of a mitochondrion.

In one embodiment, determining the first resistance to diffusion across the biological membrane with the pH gradient for the unionized species of the protonophore and determining the second resistance to diffusion across the biological membrane with the pH gradient for the ionized species of the protonophore includes: calculating the first resistance to diffusion across the biological membrane with the pH gradient for the unionized species of the protonophore as an inverse function of a first permeability across the biological membrane with the pH gradient for the unionized species of the protonophore and a function of a first ratio of a first number of molecules of the unionized species of the protonophore at a steady-state condition on a first side of the biological membrane with the pH gradient from which the unionized species of the protonophore translocates over a second number of molecules of the ionized species of the protonophore at the steady-state condition on a second opposite side of the biological membrane with the pH gradient; and calculating the second resistance to diffusion across the biological membrane with the pH gradient for the ionized species of the protonophore as an inverse function of a second permeability across the biological membrane with the pH gradient for the ionized species of the protonophore and a function of the first ratio.

In one embodiment, the first permeability across the biological membrane with the pH gradient for the unionized species of the protonophore and the second permeability across the biological membrane with the pH gradient for the ionized species of the protonophore are each determined using a parallel artificial membrane permeability assay.

In one embodiment, the first permeability across the biological membrane with the pH gradient for the unionized species of the protonophore is estimated from a first model-lipid-water partition coefficient P value for the unionized species of the protonophore combined with one or more measures of size and shape of the protonophore, and the second permeability across the biological membrane with the pH gradient for the ionized species of the protonophore is estimated from a second model-lipid-water partition coefficient P value for the unionized species of the protonophore combined with the one or more measures of size and shape of the protonophore.

In one embodiment, the first model-lipid-water partition coefficient P value for the unionized species of the protonophore is measured from an octanol-water partitioning assay to provide a first octanol-water partition coefficient P_{octanol-water} value for the unionized species of the protonophore or from a liposome-water partitioning assay to provide either a first liposome-water partition coefficient P_{liposome-water} value for the unionized species of the protonophore or a first membrane-water partition coefficient P_{membrane-water} value for the unionized species of the protonophore, and the model-lipid-water partition coefficient P value for the ionized species of the protonophore is measured from an octanol-water partitioning assay to provide a second octanol-water partition coefficient P_{octanol-water} value for the ionized species of the protonophore or from a liposome-water partitioning assay to provide either a second liposome-water partition coefficient P_{liposome-water} value for the ionized species of the protonophore or a second membrane-water partition coefficient P_{membrane-water} value for the ionized species of the protonophore.

In one embodiment, the first model-lipid-water partition coefficient P value for the unionized species of the protonophore is estimated by calculating a third octanol-water partition coefficient P_{octanol-water} value for the unionized species of the protonophore by summarizing the lipophilicity contribution or the hydrophilicity contribution of the various parts of a molecule of the unionized species of the protonophore using a Marvin program (ChemAxon Ltd), a ChemSketch program (Advanced Chemistry Development Inc.), a KowWin program (SRC, Inc.), or an EPI Suite program (United States Environmental Protection Agency), and the second model-lipid-water partition coefficient P value for the ionized species of the protonophore is estimated by calculating a fourth octanol-water partition coefficient P_{octanol-water} value for the ionized species of the protonophore by summarizing the lipophilicity contribution or the hydrophilicity contribution of the various parts of a molecule of the ionized species of the protonophore using the Marvin program, the ChemSketch program, the KowWin program, or the EPI Suite program. In one embodiment, the third octanol-water partition coefficient P_{octanol-water} value for the unionized species of the protonophore and the fourth octanol-water partition coefficient P_{octanol-water} value for the ionized species of the protonophore are calculated using the Marvin program.

In one embodiment, the one or more measures of size and shape of the protonophore include a minimal projection area of the protonophore and a z-length of the protonophore.

In one embodiment, the calculating of the minimal projection area of the protonophore and the calculating of the z-length of the protonophore are performed using the Marvin program.

In one embodiment, the estimating of the first permeability across the biological membrane with the pH gradient for the unionized species of the protonophore from the first model-lipid-water partition coefficient P value for the unionized species of the protonophore comprises: empirically determining an estimate of an optimal model-lipid-water partition coefficient P value for diffusion across the biological membrane with the pH gradient; and provided that the first model-lipid-water partition coefficient P value for the unionized species of the protonophore is equal to the estimated optimal model-lipid-water partition coefficient P value for diffusion across the biological membrane with the pH gradient, then the first permeability across the biological membrane with the pH gradient for the unionized species of the protonophore is multiplied by 1; or the first model-lipid-water partition coefficient P value for the unionized species of the protonophore is not equal to the estimated optimal model-lipid-water partition coefficient P value for diffusion across the biological membrane with the pH gradient, then the first permeability across the biological membrane with the pH gradient for the unionized species of the protonophore is divided by a factor equal to 10 to the power of the absolute value of the difference between the log₁₀-transformed first model-lipid-water partition coefficient P value for the unionized species of the protonophore and the log₁₀-transformed estimated optimal model-lipid-water partition coefficient P value for diffusion across the biological membrane with the pH gradient.

In one embodiment, the estimating of the second permeability across the biological membrane with the pH gradient for the ionized species of the protonophore from the second model-lipid-water partition coefficient P value for the ionized species of the protonophore comprises: empirically determining an estimate of an optimal model-lipid-water partition coefficient P value for diffusion across the biological membrane with the pH gradient; and provided that the second model-lipid-water partition coefficient P value for the ionized species of the protonophore is equal to the estimated optimal model-lipid-water partition coefficient P value for diffusion across the biological membrane with the pH gradient, then the second permeability across the biological membrane with the pH gradient for the ionized species of the protonophore is multiplied by 1; or the second model-lipid-water partition coefficient P value for the ionized species of the protonophore is not equal to the estimated optimal model-lipid-water partition coefficient P value for diffusion across the biological membrane with the pH gradient, then the second permeability across the biological membrane with the pH gradient for the ionized species of the protonophore is divided by a factor equal to 10 to the power of the absolute value of the difference between the log₁₀-transformed second model-lipid-water partition coefficient P value for the ionized species of the protonophore and the log₁₀-transformed estimated optimal model-lipid-water partition coefficient P value for diffusion across the biological membrane with the pH gradient.

In one embodiment, the estimating of the first permeability across the biological membrane with the pH gradient for the unionized species of the protonophore from the one or more measures of size and shape of the protonophore and the estimating of the second permeability across the biological membrane with the pH gradient for the ionized species of the protonophore from the one or more measures of size and shape of the protonophore comprise: provided that the minimal projection area of the protonophore is equal to the minimal projection area of the reference protonophore, and that the z-length of the protonophore is equal to the z-length of the reference protonophore, then the first permeability across the biological membrane with the pH gradient for the unionized species of the protonophore and the second permeability across the biological membrane with the pH gradient for the ionized species of the protonophore are multiplied by a factor of 1; or if the minimal projection area of the protonophore is not equal to the minimal projection area of the reference protonophore, then the first permeability across the biological membrane with the pH gradient for the unionized species of the protonophore and the second permeability across the biological membrane with the pH gradient for the ionized species of the protonophore are divided by a factor equal to the square of the ratio of the minimal projection area of the reference protonophore over the minimal projection area of the protonophore; and if the z-length of the protonophore is not equal to the z-length of the reference protonophore, then the first permeability across the biological membrane with the pH gradient for the unionized species of the protonophore and the second permeability across the biological membrane with the pH gradient for the ionized species of the protonophore are divided by a factor equal to the ratio of the z-length of the reference protonophore over the z-length of the protonophore, or if the z-length of the protonophore is not equal to the z-length of the reference protonophore, then the first permeability across the biological membrane with the pH gradient for the unionized species of the protonophore and the second permeability across the biological membrane with the pH gradient for the ionized species of the protonophore are divided by a factor equal to the ratio of the z-length of the reference protonophore over the z-length of the protonophore.

In one embodiment, determining the first ratio of the number of molecules of the unionized species of the protonophore at the steady-state condition on the first side of the biological membrane with the pH gradient from which the unionized species of the protonophore translocates over the number of molecules of the ionized species of the protonophore at the steady-state condition on the second opposite side of the biological membrane with the pH gradient is performed using a first algorithm.

In one embodiment, the first algorithm includes: (1) determining an acid-dissociation constant pKa for each ionizable site of the protonophore; (2) determining from the acid-dissociation constant pKa for each ionizable site of the protonophore a first proportions of the unionized and of the ionized species of the protonophore at a first pH of the first side of the biological membrane with the pH gradient and a second proportions of the unionized and of the ionized species of the protonophore at a second pH of the second opposite side of the biological membrane with the pH gradient; (3) independently for each pair of the unionized species of the protonophore and one of the ionized species of the protonophore, calculating a second ratio of the concentration of the unionized species of the protonophore on the first side of the biological membrane with the pH gradient over the concentration of the unionized species of the protonophore on the second opposite side of the biological membrane with the pH gradient at the steady-state condition, and calculating a third ratio of the concentration of the ionized species of the protonophore on the first side of the biological membrane with the pH gradient over the concentration of the ionized species of the protonophore on the second opposite side of the biological membrane with the pH gradient at the steady-state condition; (4) independently for each pair of the unionized species of the protonophore and one of the ionized species of the protonophore, calculating a fourth ratio of the number of molecules of the unionized species of the protonophore at the steady-state condition on the first side of the biological membrane with the pH gradient from which the unionized species of the protonophore translocates over the number of molecules of the ionized species of the protonophore at the steady-state condition on the second opposite side of the biological membrane with the pH gradient on the basis of the second and third ratios; (5) provided that the fourth ratio is greater than 1, then, independently for each pair of the unionized species of the protonophore and one of the ionized species of the protonophore, considering the resistance to diffusion across the biological membrane with the pH gradient of the unionized species of the protonophore to be a native resistance to diffusion across the biological membrane with the pH gradient of the unionized species of the protonophore, and incrementally reducing the native resistance to diffusion across the biological membrane with the pH gradient of the unionized species of the protonophore to provide with each increment an adjusted resistance to diffusion across the biological membrane with the pH gradient of the unionized species of the protonophore, not altering the resistance to diffusion across the biological membrane with the pH gradient of the ionized species, and considering this to constitute the first part of the process of incremental reduction of native resistance; or (6) provided that the fourth ratio is smaller than 1, then, independently for each pair of the unionized species of the protonophore and one of the ionized species of the protonophore, considering the resistance to diffusion across the biological membrane with the pH gradient of the ionized species of the protonophore to be a native resistance to diffusion across the biological membrane with the pH gradient of the ionized species of the protonophore, incrementally reducing the native resistance to diffusion across the biological membrane with the pH gradient of the ionized species of the protonophore to provide with each increment an adjusted resistance to diffusion across the biological membrane with the pH gradient of the ionized species of the protonophore, not altering the resistance to diffusion across the biological membrane with the pH gradient of the unionized species, and considering this to constitute the first part of the process of incremental reduction of native resistance; (7) independently for each pair of the unionized species of the protonophore and one of the ionized species of the protonophore, after each incremental reduction of either the native resistance to diffusion across the biological membrane with the pH gradient of the unionized species of the protonophore or of the native resistance to diffusion across the biological membrane with the pH gradient of the ionized species of the protonophore, recalculating the second ratio to provide a fifth ratio, recalculating the third ratio to provide a sixth ratio, recalculating the fourth ratio on the basis of the fifth ratio and of the sixth ratio to provide a seventh ratio, and considering this to constitute the second part of the process of incremental reduction of native resistance; and (8) provided that the fourth ratio is greater than 1, then, independently for each pair of the unionized species of the protonophore and one of the ionized species of the protonophore, repeating both the first and second parts of the process of incremental reduction of native resistance until such time as the seventh ratio is numerically equal to an eigth ratio of the adjusted resistance to diffusion across the biological membrane with the pH gradient of the unionized species of the protonophore over the native resistance to diffusion across the biological membrane with the pH gradient of the unionized species of the protonophore, and taking the first ratio to be equal to the seventh ratio at the point of the final iteration of both the first and the second parts of the process of incremental reduction of the native resistance to diffusion across the biological membrane with the pH gradient of the unionized species of the protonophore; or (9) provided that the fourth ratio is smaller than 1, then, independently for each pair of the unionized species of the protonophore and one of the ionized species of the protonophore, repeating both the first and second parts of the process of incremental reduction of native resistance until such time as the seventh ratio is numerically equal to a ninth ratio of the adjusted resistance to diffusion across the biological membrane with the pH gradient of the ionized species of the protonophore over the native resistance to diffusion across the biological membrane with the pH gradient of the ionized species of the protonophore, and taking the first ratio to be equal to the seventh ratio at the point of the final iteration of both the first and the second parts of the process of incremental reduction of the native resistance to diffusion across the biological membrane with the pH gradient of the ionized species of the protonophore.

In one embodiment, the calculating the first resistance to diffusion across the biological membrane with the pH gradient for the unionized species of the protonophore and the second resistance to diffusion across the biological membrane with the pH gradient for the ionized species of the protonophore as the function of the first ratio includes: provided that the fourth ratio is greater than 1, then the first resistance to diffusion across the biological membrane with the pH gradient for the unionized species of the protonophore is taken to be equal to the inverse of the first permeability divided by the first ratio, and the second resistance to diffusion across the biological membrane with the pH gradient for the ionized species of the protonophore is taken to be the inverse of the second permeability; provided that the fourth ratio is smaller than 1, then the second resistance to diffusion across the biological membrane with the pH gradient for the ionized species of the protonophore is taken to be the inverse of the second permeability divided by the first ratio, and the first resistance to diffusion across the biological membrane with the pH gradient for the unionized species of the protonophore is taken to be the inverse of the first permeability; and provided that the fourth ratio is equal to 1, then the first resistance to diffusion across the biological membrane with the pH gradient for the unionized species of the protonophore is taken to be the inverse of the first permeability, and the second resistance to diffusion across the biological membrane with the pH gradient for the ionized species of the protonophore is taken to be the inverse of the second permeability.

In one embodiment, the pKa of each ionizable site of the protonophore is measured by a titration assay.

In one embodiment, the pKa of each ionizable site of the protonophore is calculated by summarizing the partial charge distribution over the various parts of a molecule of the protonophore using the Marvin program.

In one embodiment, the first proportions of the unionized and of the ionized species of the protonophore at the first pH of the first side of the biological membrane with the pH gradient and the second proportions of the unionized and of the ionized species of the protonophore at the second pH of the second side of the biological membrane with the pH gradient are calculated from the pKa of each of the ionizable sites of the protonophore by applying the Henderson-Hasselbalch equation. In one embodiment, applying of the Henderson-Hasselbalch equation is performed using the Marvin program.

In one embodiment, calculating the second and fifth ratios of the concentration of the unionized species of the protonophore on the first side of the biological membrane with the pH gradient over the concentration of the unionized species of the protonophore on the second opposite side of the biological membrane with the pH gradient at the steady-state condition and calculating the third and sixth ratios of the concentration of the ionized species of the protonophore on the first side of the biological membrane with the pH gradient over the concentration of the ionized species of the protonophore on the second opposite side of the biological membrane with the pH gradient at the steady-state condition are performed by applying the Nernst equation, provided that: (1) the translocation of molecules of the unionized species across the biological membrane with the pH gradient is considered to be coupled to the translocation of molecules of the ionized species across the biological membrane with the pH gradient in a direction opposite that of the translocation of molecules of the unionized species; (2) the translocation of molecules of the unionized species across the biological membrane with the pH gradient and the coupled translocation of molecules of the ionized species across the biological membrane with the pH gradient in the direction opposite that of molecules of the unionized species are considered to be both driven by a second sum of a chemical potential energy released by a concurrent translocation of protons across the biological membrane with the pH gradient from a side of low pH of the biological membrane with the pH gradient to a side of high pH of the biological membrane with the pH gradient plus an electrical potential energy released by the concurrent translocation of protons across the biological membrane with the pH gradient from the side of low pH of the biological membrane with the pH gradient to the side of high pH of the biological membrane with the pH gradient; (3) the first proportions of the unionized species and of the ionized species of the protonophore at the first pH of the first side of the biological membrane with the pH gradient and the second proportions of the unionized species and of the ionized species of the protonophore at the second pH of the second opposite side of the biological membrane with the pH gradient are considered to be constants; (4) independently for each pair of the unionized species of the protonophore and one of the ionized species of the protonophore, the second sum is considered to be partitioned between the unionized species and the ionized species of the protonophore such that a first quotient of the amount of the second sum partitioned to the unionized species of the protonophore divided by the resistance to diffusion across the biological membrane with the pH gradient of the unionized species of the protonophore is equal to a second quotient of the amount of the second sum partitioned to the ionized species of the protonophore divided by the resistance to diffusion across the biological membrane with the pH gradient of the ionized species of the protonophore; (5) independently for each pair of the unionized species of the protonophore and one of the ionized species of the protonophore, provided that only one of the unionized species of the protonophore or the ionized species of the protonophore exhibits a net electrical charge, then the electrical potential energy released by the translocation of protons across the biological membrane with the pH gradient from the side of low pH of the biological membrane with the pH gradient to the side of high pH of the biological membrane with the pH gradient is considered to be not partitioned between the unionized species of the protonophore and the ionized species of the protonophore, but instead considered to be attributed completely to the species that exhibits a net electrical charge; and (6) independently for each pair of the unionized species of the protonophore and one of the ionized species of the protonophore, provided that both the unionized species of the protonophore and the ionized species of the protonophore exhibit a net electrical charge, then the electrical potential energy released by the translocation of protons across the biological membrane with the pH gradient from the side of low pH of the biological membrane with the pH gradient to the side of high pH of the biological membrane with the pH gradient is considered to be partitioned between the unionized species of the protonophore and the ionized species of the protonophore in proportion to the absolute value of the net electrical charge of each species.

The present invention also provides novel protonophores, which have estimated activities that correspond to an U₅₀ of about 20 μM or less.

The present invention provides a method of treating a disorder, disease, or condition benefiting from an uncoupling of mitochondrial respiration in a patient in need thereof. The method includes: administering a composition including a protonophore of Formula (I)

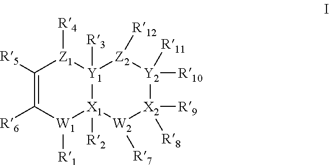

wherein: W₁ is carbon, oxygen, sulfur, or nitrogen; X₁, Y₁, and Z₁ are each independently carbon; R′₁ is absent, hydroxyl, or thiol; R′₂ is hydrogen, (C1-C6)aldehyde, (C1-C8)alkyl, acetyl, amino, (C1-C6)dialkylamino, or (C1-C8)alkenyl; R′₃ is hydrogen, hydroxyl, thiol, (C1-C6)aldehyde, (C1-C8)alkyl, or (C1-C8)alkenyl; R′₄ is hydrogen, (C1-C6)aldehyde, hydroxyl, (C1-C8)alkyl, (C1-C6)dialkylamino, (C1-C8)alkyl, acetyl, (C1-C8)alkenyl, CO(C1-C8)alkenyl, CO(C1-C8)alkyl, or CO-p-C₆H₅SH; R′₅ is hydrogen, (C1-C6)aldehyde, (C1-C8)alkyl, thiol, acetyl, (C1-C8)alkenyl, or CO(C1-C8)alkenyl; and R′₆ is hydrogen, amino, (C1-C6)dialkylamino, (C1-C6)aldehyde, (C1-C8)alkyl, (C1-C6)alkoxy, acetyl, (C1-C8)alkenyl, CO(C1-C8)alkenyl, or CO(C1-C8)alkyl, provided that W₂, R′₇, X₂, R′₈, R′₉, Y₂, R′₁₀, R′₁₁, Z₂, and R′₁₂ are absent; or

wherein: W₁, X₁, Y₁, and Z₁ are each independently carbon; W₂ is oxygen, carbon, or nitrogen; X₂ is carbon or nitrogen; Y₂ and Z₂ are each independently carbon; R′₁ is hydrogen, (C1-C6)aldehyde, (C1-C8)alkyl, acetyl, (C1-C8)alkenyl, CO(C1-C8)alkenyl, or CO(C1-C8)alkyl; R′₂ and R′₃ are each independently absent; R′₄ is hydrogen, (C1-C6)aldehyde, (C1-C8)alkyl, acetyl, or (C1-C8)alkenyl; R′₅ is hydrogen, hydroxyl, thiol, amino, (C1-C8)alkyl, (C1-C6)aldehyde, acetyl, (C1-C8)alkylamino, (C1-C8)alkenyl, CO(C1-C8)alkenyl, or CO(C1-C8)alkyl; R′₆ is hydrogen, hydroxyl, (C1-C6)aldehyde, thiol, (C1-C8)alkyl, amino, (C1-C8)alkylamino, acetyl, (C1-C8)alkenyl, CO(C1-C8)alkenyl, or CO(C1-C8)alkyl; R′₇ is absent, hydrogen, (C1-C6)aldehyde, amino, or (C1-C8)alkylamino; R′₈ is absent, hydrogen, acetyl, (C1-C8)alkyl, amino, (C1-C8)alkylamino, (C1-C8)alkenyl, or CO(C1-C8)alkenyl or R′₈, R′₉, R′₁₀ and R′₁₁ together form two carbon atoms of an unsubstituted or substituted(C₁-C₁₂)aryl or two carbon atoms of an unsubstituted or substituted(C₁-C₁₂)heteroaromatic; R′₉ and R′₁₁ are each independently absent or hydrogen; R′₁₀ is hydrogen, hydroxyl, thiol, amino, (C1-C8)alkylamino, (C1-C8)alkyl, or (C1-C8)alkenyl; R′₁₂ is carbonyl, hydrogen, amino, (C1-C8)alkylamino, or (C1-C8)alkyl; or

wherein: W₁ is oxygen, sulfur, carbon, or nitrogen; X₁ and Y₁ are each independently carbon or nitrogen; Z₁ is absent; W₂ and Z₂ are each independently hydrogen; R′₁ is absent, hydrogen, or carbonyl; R′₂ is absent, hydrogen, thiol, hydroxyl, (C1-C8)alkyl, acetyl, (C1-C8)alkenyl, CO(C1-C8)alkenyl, CO(C1-C8)alkyl; or CO—(C1-C6)aryl; R′₃ is absent, hydrogen, thiol, hydroxyl, (C1-C6)aldehyde, (C1-C8)alkyl, acetyl, CO(C1-C8)alkenyl, or CO(C1-C8)alkyl; R′₄ is absent; R′₅ is hydrogen, (C1-C8)alkyl, (C1-C8)alkenyl, or CO(C1-C8)alkenyl; or R′₅ and R′₆ together form two carbon atoms of an unsubstituted or substituted(C₁-C₁₂)aryl; R′₆ is hydroxyl, (C1-C6)aldehyde, acetyl, (C1-C8)alkyl, (C1-C8)alkenyl, CO(C1-C8)alkenyl, or CO(C1-C8)alkyl; W₂, X₂, Y₂, Z₂, R′₇, R′₈, R′₉, R′₁₀, R′₁₁, and R′₁₂ is absent; and R′₂, R′₃, Z₂, W₂, X₂, Y₂, Z₂, R′₇, R′₈, R′₉, R′₁₀, R′₁₁, and R′₁₂ together form two carbon atoms of an unsubstituted or substituted(C₁-C₁₂)aryl; or

wherein: W₁ is oxygen; X₁, Y₁, and Z₁ are each independently carbon; R′₁, R′₂, and R′₃ are each independently absent; R′₄ and R′₆ are each independently (C1-C8)alkyl; R′₅ is hydroxyl; W₂ and Z₂ are each independently CH; X₂ and Y₂ are each independently carbon; R′₇ and R′₁₂ are each independently hydrogen; and R′₈, R′₉, R′₁₀ and R′₁₁ together form two carbon atoms of an unsubstituted or substituted(C1-C12)heteroaromatic, or a pharmaceutically acceptable salt, solvate, or prodrug thereof.

In one embodiment, the disorder, disease or condition includes insulin resistance, impaired glucose tolerance, Type I diabetes, Type II diabetes, fatty liver disease, lipid accumulation in striated muscle, hyperglycemia, hyperinsulinemia, cancer, or a combination thereof.

In one embodiment, the disorder, disease or condition includes insulin resistance, impaired glucose tolerance, Type I diabetes, Type II diabetes, fatty liver disease, lipid accumulation in striated muscle, hyperglycemia, hyperinsulinemia, cancer, insulin resistance syndrome, metabolic syndrome, cardiomyopathy, atherosclerosis, vascular disease, coronary heart disease, microvascular disease, hypertension, diabetic nephropathy, diabetic neuropathy, diabetic retinopathy, deficient satiety signaling, low oxidative capacity, or a combination thereof.

In one embodiment, W₁ is C, O, S, or N; X₁, Y₁, and Z₁ are each independently carbon; R′₁ is Absent, OH, or SH; R′₂ is Hydrogen, CHO, CH₃, COCH₃, C(CH₃)₃, NH₂, N(CH₃)₂, CH═CH₂, CH═CHCH₃, CH═C(CH₃)₂, CH═CHCH═CH₂, or (CH═CH)₂CH═CH₂; R′₃ is Hydrogen, OH, SH, CHO, CH₃, CH═CH₂, CH═C(CH₃)₂ or (CH═CH)₂(CH₃); R′₄ is Hydrogen, CHO, OH, CH₃, N(CH₃)₂, C(CH₃)₃, COCH₃, CH═CH₂, CH═CHCH₃, (CH═CH)₂(CH₃), CH═CH₂CH═CH₂, COCH═CH₂, CO(CH═CH)₃(CH₃), CO(CH═CH)₂CH═CH₂, CO(CH₂)₄CH₃, or CO-p-C₆H₅SH; R′₅ is Hydrogen, CHO, CH₃, SH, COCH₃, C(CH₃)₃, CH═CH₂, CH═CHCH₃, CH═C(CH₃)₂ (CH═CH)₄(CH₃), or COCH═CH₂; and R′₆ is Hydrogen, NH₂, N(CH₃)₂, CHO, CH₃, OCH₃, COCH₃, CH═CH₂, CH═C(CH₃)₂, COCH═CH₂, or CO(CH₂)₇CH₃; provided that W₂, R′₇, X₂, R′₈, R′₉, Y₂, R′₁₀, R′₁₁, Z₂, and R′₁₂ are absent.

In one embodiment, W₁, X₁, Y₁, and Z₁ are each independently C; W₂ is O, C, or N; X₂ is C or N; X₂ and Y₂ are each independently C; R′₁ is Hydrogen, CHO, CH₃, COCH₃, CH═CH₂, CH═CH(CH₃), (CH═CH)₂CH₃, (CH═CH)₂CH═CH₂, (CH═CH)₃CH₃, COCH═CH₂, COCH═CHCH₃, or CO(CH₂)₃CH₃; R′₂ and R′₃ are each independently absent; R′₄ is Hydrogen, CHO, CH₃, COCH₃, or CH═CH₂; R′₅ is Hydrogen, OH, SH, NH₂, CH₃, CHO, COCH₃, NHCH₃, CH═CH₂, CH═CH(CH₃), C(CH₃)═CH(CH₃), CH═CHCH═CH₂, (CH═CH)₂CH═CH₂, (CH═CH)₃CH₃, COCH═CH₂, COCH═CHCH₃, CO(CH₂)₂CH₃, or CO(CH═CH)₂CH₃; R′₆ is Hydrogen, OH, CHO, SH, CH₃, NH₂, NHCH₃, COCH₃, CH═CH₂, CH═CHCH₃, CH═CHCH═CH₂, (CH═CH)₂(CH₃), COCH═CHCH₃, CO(CH₂)₃CH₃, or CO(CH₂)₄CH₃; R′₇ is Absent, Hydrogen, CHO, NH₂, or NHCH₃; R′₈ is Absent, Hydrogen, COCH₃, CH₃, NH₂, NHCH₃, CH═CH₂, CH═CH(CH₃), CH═C(CH₃)₂, CH═CHCH═CH₂, (CH═CH)₃CH₃, CH═CHCH═CH₂, (CH═CH)₂CH═CH₂, COCH═CH₂, COCH═C(CH₃)₂, COCH═CH(CH₃), or R′₈, R′₉, R′₁₀ and R′₁₁ together form the 3 and 4 carbon atoms of 2-carboxaldehyde-phenol, the 3 and 4 carbon atoms of 2-carboxaldehyde-6-methyl-phenol, the 3 and 4 carbon atoms of 2-acetyl-6-methyl-phenol, the 4 and 5 carbon atoms of 2-carboxaldehyde-phenol, the 3 and 4 carbon atoms of 2-carboxaldehyde-phenol, the 3 and 4 carbon atoms of 2-carboxaldehyde-6-methyl-phenol, the 3 and 4 carbon atoms of 2-carboxaldehyde-phenol, the 1 and 2 carbon atoms of phenyl, or the 2 and 3 carbon atoms of [2,3-b]1-H pyrole; R′₉ and R′₁₁ are each independently absent or hydrogen; R′₁₀ is Hydrogen, OH, SH, NH₂, NHCH₃, CH₃, CH═CH₂, or CH═CHCH₃; and R′₁₂ is ═O, Hydrogen, NH₂, NHCH₃, or CH₃.

In one embodiment, W₁ is O, S, C, or N; X₁ and Y₁ are each independently C or N; Z₁ is Absent; W₂ and Z₂ are each independently Hydrogen; R′₁ is Absent, Hydrogen, or ═O; R′₂ is Absent, Hydrogen, SH, OH, CH₃, C(CH₃)₃, COCH₃, CH═CH₂, CH═CH(CH₃), CH═C(CH₃)₂, (CH═CH)₂CH₃, (CH═CH)₃CH₃, COCH═CH₂, COCH═CH(CH₃), CO(CH₂)₃CH₃, CO(CH₂)₇CH₃, or CO-phenyl: R′₃ is Absent, Hydrogen, SH, OH, CHO, CH₃, C(CH₃)₃, COCH₃, COCH═CH₂, COCH═CH(CH₃), CO(CH₂)₃CH₃, or CO(CH₂)₅CH₃; R′₄ is Absent; R′₅ is Hydrogen, CH₃, C(CH₃)₃, CH═CH₂, CH═CH(CH₃), CH═C(CH₃)₂, CH═CHCH═CH₂, (CH═CH)₂CH₃, (CH═CH)₃CH₃, or COCH═CH₂; or R′₅ and R′₆ together form the 5 and 6 carbon atoms of 1-acetyl-2-pentadienyl-[5,6-b]phenyl, the 3 and 4 carbon atoms of [3,4-b]methylphenyl, the 4 and 5 carbon atoms of [5,6-b]1-methyl-2-vinyl-phenyl, the 1 and 2 carbon atoms of [1,2-b]phenyl, the 3 and 4 carbon atoms of [3,4-b]1-propenyl-phenyl, the 4 and 5 carbon atoms of [4,5-b]-1-acryl-2-methyl-phenyl, the 4 and 5 carbon atoms of [4,5-b]-1-methacryl-2-methyl-phenyl, the 3 and 4 carbon atoms of [3,4-b]-1-(2-methyl-1-propenyl)phenyl, the 4 and 5 carbon atoms of [4,5-b]-1,2-dimethylphenyl, the 4 and 5 carbon atoms of [4,5-b]-1,3 diacetyl-2-(1-propenyl)-phenyl, the 2 and 3 carbon atoms of [2,3-b]-1-(pentane-acyl)-phenyl, the 2 and 3 carbon atoms of [2,3-b]-1-carboxaldehyde-phenyl, the 3 and 4 carbon atoms of [3,4-b]-1-pentane-acyl-phenyl, the 3 and 4 carbon atoms of [3,4-b]-1-acyl-phenyl, the 5 and 6 carbon atoms of [5-6, b]-2-methyl-3-vinyl-4-carboxaldehyde-phenol, the 5 and 6 carbon atoms of [5-6, b]-2-vinyl-4-acetyl-phenol, the 5 and 6 carbon atoms of [5-6, b]-2-vinyl-4-methacryl-phenol, the 5 and 6 carbon atoms of [5-6, b]-2-acetyl-phenol, the 5 and 6 carbon atoms of [5-6, b]-2-acetyl-4-vinyl-phenol, the 2 and 3 carbon atoms of [2-3, b]-1,4-dicarboxyaldehyde-phenyl, the 5 and 6 carbon atoms of [5-6, b]-1,4-dicarboxylaldehyde-2-methyl-phenyl, the 5 and 6 carbon atoms of [5-6, b]-2,3-dimethyl-1-carboxylaldehyde-phenyl, the 4 and 5 carbon atoms of [4-5, b]-2,6-dicarboxylaldehyde-3-methyl-phenol, the 4 and 5 carbon atoms of [4-5, b]-2,6-dicarboxylaldehyde-3-vinyl-phenol, the 4 and 5 carbon atoms of [4-5, b]-2-acetyl-6-propylcarboxy-phenol, the 4 and 5 carbon atoms of [4-5, b]-2-carboxaldehyde-6-methacryl-phenol, the 4 and 5 carbon atoms of [4-5, b]-2-acetyl-6-methacryl-phenol, the 4 and 5 carbon atoms of [4-5, b]-6-carboxylaldehyde-2-methyl-thiophenol, the 3 and 4 carbon atoms of [3-4, b]-thiophenol, the 4 and 5 carbon atoms of [4-5, b]-2-methyl-thiophenol, the 4 and 5 carbon atoms of [4-5, b]-2-methyl-6-methacryl-thiophenol, the 3 and 4 carbon atoms of [3-4, b]-2,6-diacetyl-thiophenol, the 3 and 4 carbon atoms of [3-4, b]-2-acetyl, 6-hexanylcarbonyl-thiophenol, the 3 and 4 carbon atoms of [3-4, b]-6-pentanylcarbonyl-thiophenol, the 3 and 4 carbon atoms of [3-4, b]-2,5-divinyl-6-carboxaldehyde-thiophenol, the 4 and 5 carbon atoms of [4-5, b]-2-propenyl-thiophenol, the 4 and 5 carbon atoms of [4-5, b]-6-pentenyl-2-carboxaldehyde-thiophenol, the 2 and 3 carbon atoms of [2-3, b]-2-(6-pentylcarbonyl)-thiophenol, the 3 and 4 carbon atoms of [3-4, b]-6-pentanylcarbonyl-thiophenol, the 2 and 3 carbon atoms of [2-3, b]-2-carboxaldehyde-1-phenol, or the 2 and 3 carbon atoms of [2,3-b]-1-carboxaldehyde-5,7-divinyl-6-methyl-napthane; R′₆ is OH, CHO, COCH₃, CH₃, CH═CH₂, CH═CH(CH₃), CO(CH═CH)₃CH₃, or CO(CH₂)₅CH₃; W₂, X₂, Y₂, Z₂, R′₇, R′₈, R′₉, R′₁₀, R′₁₁, and R′₁₂ is Absent; and R′₂, R′₃, Z₁, W₂, X₂, Y₂, Z₂, R′₇, R′₈, R′₉, R′₁₀, R′₁₁, and R′₁₂ together form the 3 and 4 carbon atoms of [3-4, d]-2-carboxaldehyde-1-phenol, the 3 and 4 carbon atoms of [3-4, d]-2-carboxaldehyde-1-phenol, or the 4 and 5 carbon atoms of [4,5-d]-2-carboxaldehyde-1-phenol.

In one embodiment, W₁ is O; X₁, Y₁, and Z₁ are each independently C; R′₁ is Absent; R′₂ and R′₃ are each independently absent; R′₄ and R′₆ are each independently CH₃; R′₅ is OH; W₂ and Z₂ are each independently CH; X₂ and Y₂ are each independently C; R′₇ and R′₁₁ are each independently hydrogen; and R′₈, R′₉, R′₁₀ and R′₁₁ together form the 5 and 6 carbon atoms of [5,6-e]2,4-dimethyl-4-hydroxyl-pyran.

In one embodiment, the protonophore of Formula (I) is represented by a protonophore of Formula (II)

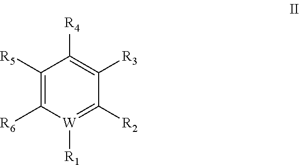

wherein: W is C, O, S, or N; R₁ is Absent, OH, or SH; R₂ is Hydrogen, NH₂, CH₃, C(CH₃)₃, COCH₃, CHO, CH═CH₂, CH═CHCH₃, CH═CHCH═CH₂, (CH═CH)₂(CH₃), (CH═CH)₂CH═CH₂, or (CH═CH)₂CH═CH₂; R₃ is Hydrogen, OH, SH, CH₃, CHO, CH═CH₂, CH═C(CH₃)₂, (CH═CH)₂(CH₃), or (CH═CH)₃(CH₃); R₄ is CHO, CH₃, C(CH₃)₃, N(CH₃)₂, COCH₃, CH═CH₂, CH═CHCH₃, CH═CH₂CH═CH₂, (CH═CH)₂(CH₃), COCH═CH₂, CO(CH═CH)₂CH═CH₂, CO(CH═CH)₃(CH₃), or CO-p-C₆H₅SH; R₅ is Hydrogen, CHO, CH₃, SH, COCH₃, C(CH₃)₃, CH═CH₂, CH═CHCH₃, CH═C(CH₃)₂ (CH═CH)₄(CH₃), or COCH═CH₂; and R₆ is Hydrogen, NH₂, N(CH₃)₂, CHO, CH₃, OCH₃, COCH₃, CH═CH₂, CH═C(CH₃)₂, COCH═CH₂, or CO(CH₂)₇CH₃.

In one embodiment, the protonophore is: 1,3-dihydroxy, 2-(propen-1-yl), 3,6-diformyl, benzene, 1,3-dihydroxy, 4,6-di(prop-2-en-1-one), benzene,1,3-dihydroxy, 2,5-diethenyl, 4,6-diacetyl, benzene,1,3-dihydroxy, 2-((1E)-buta-1,3-dien-1-yl), 4,6-acetyl, benzene,2,4-diacetyl, 34(1E,3E,5E)-hepta-1,3,5-trien-1-yl), thiophenol, 2,4,6-triformyl, 3-methyl, 5-tert-butyl, thiophenol, 2,4-diformyl, 3-((1E,3E)-penta-1,3-dien-1-yl), thiophenol, 3,5-diformyl, 4-((1E,3E)-penta-1,3-dien-1-yl), thiophenol, 2,4,6-triformyl, 3-((1E,3E,5E)-hepta-1,3,5-trien-1-yl), thiophenol, 2,6-diformyl, 4-((2E,4E,6E)-octa-2,4,6-trien-1-one), thiophenol, 2-formyl, 4-((2E,4E,6E)-hepta-2,4,6-trien-1-one), thiophenol, 2-acetyl, 4-(hexan-1-one), thiophenol, 2-ethenyl, 3-sulfanyl, 5-(prop-2-en-1-one), thiophenol, 2-((1E,3E)-hexa-1,3,5-trien-1-yl), 3-sulfanyl, 4,6-diacetyl, thiophenol, 2,5,6-trimethyl, 3-sulfanyl, 4-acetyl, thiophenol, 2-methyl, 3-sulfanyl, 4-formyl, 6-ethenyl, thiophenol, 2-((1E,3E)-penta-1,3-dien-1-yl), 3-sulfanyl, 5-acetyl, thiophenol, 2,5,6-trimethyl, 3-sulfanyl, 4-formyl, thiophenol, 4-[(4-sulfanylphenyl)carbonyl]benzenethiol, 1,3,5-trisulfanyl, 2,4-dimethyl, 6-methoxy, benzene,1,3,5-trisulfanyl, 2,4-dimethyl, benzene, 1,3,5-trisulfanyl, 4-(propen-1-yl), benzene, 3-hydroxy, 4-[(1E)-buta-1,3-dien-1-yl], 5-methyl, pyrilium, 3-hydroxy, 4,5-diethenyl, 6-methyl, pyrilium, 3-hydroxy, 4,5-diethenyl, pyrilium, 3-hydroxy, 4-(propen-1-yl), 5-ethenyl, pyrilium, 2,4-dimethyl, 3-hydroxy, 5-[(1E,3E,5E,7E)-nona-1,3,5,7-tetraen-1-yl], 6-formyl, thiopyran, 2,4-tert-butyl, 3-hydroxy, 5-methyl, 6-formyl, thiopyran, 2,4,5-tri-(propen-1-yl), 3-hydroxy, 6-formyl, 27-4-thiopyran, 2,4-dimethyl, 3-hydroxy, 6-(nonan-1-one), thiopyran, 4-N,4-N-dimethyl, 2,4,6-triamine, 3,5-di-(2-methylpropen-1-yl), pyridine, 2-N,2-N,4-N,4-N,6-N,6-N-hexamethy, 2,4,6-triamine, 3,5-dimethyl, pyridine, 2,6-di-(2-methylpropen-1-yl), 3,5-diethenyl, 4-hydroxy, pyridine, or 2,3,4,5,6-pentaethenyl, pyridine.

In one embodiment, the protonophore of Formula (I) is represented by a protonophore of Formula (III)

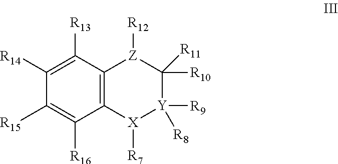

wherein: X is O, C, or N; Y is C or N; Z is C; R, is Absent, Hydrogen, CHO, ═O, NH₂, or NHCH₃, provided that when X is O, then R, is Absent, or when X is C, then R, is Hydrogen, CHO, ═O, NH₂, or NHCH₃ or when X is N, then R, is Absent or Hydrogen; R₈ is Hydrogen, CH₃, NH₂, NHCH₃, COCH₃, CH═CH₂, CH═CHCH₃, CH═C(CH₃)₂, CH═CHCH═CH₂, (CH═CH)₂CH═CH₂, (CH═CH)₃CH₃, COCH═CH₂, COCH═CH(CH₃), COCH═C(CH₃)₂, or R₈, R₉, R₁₀ and R₁₁ together form the 3 and 4 carbon atoms of 2-carboxaldehyde-phenol, the 3 and 4 carbon atoms of 2-carboxaldehyde-6-methyl-phenol, the 3 and 4 carbon atoms of 2-acetyl-6-methyl-phenol, the 4 and 5 carbon atoms of 2-carboxaldehyde-phenol, the 3 and 4 carbon atoms of 2-carboxaldehyde-phenol, the 3 and 4 carbon atoms of 2-carboxaldehyde-6-methyl-phenol, the 3 and 4 carbon atoms of 2-carboxaldehyde-phenol, the 1 and 2 carbon atoms of phenyl, or the 2 and 3 carbon atoms of [2,3-b]1-H pyrole; R₉ and R₁₁ are each independently Absent or Hydrogen; R₁₀ is Hydrogen, OH, SH, NH₂, CH₃, NHCH₃, CH═CH₂, or CH═CH(CH₃); R₁₂ is ═O, Hydrogen, NH₂, NHCH₃, or CH₃; R₁₃ is Hydrogen, CHO, CH₃, COCH₃, or CH═CH₂; R₁₄ is Hydrogen, OH, SH, NH₂, CH₃, CHO, COCH₃, NHCH₃, CH═CH₂, CH═CH(CH₃), C(CH₃)═CH(CH₃), CH═CHCH═CH₂, (CH═CH)₂CH═CH₂, (CH═CH)₃CH₃, COCH═CH₂, COCH═CHCH₃, CO(CH₂)₂CH₃, or CO(CH═CH)₂CH₃; R₁₅ is Hydrogen, OH, CHO, SH, CH₃, NH₂, NHCH₃, COCH₃, CH═CH₂, CH═CHCH₃, CH═CHCH═CH₂, (CH═CH)₂(CH₃), COCH═CHCH₃, CO(CH₂)₃CH₃, or CO(CH₂)₄CH₃; and R₁₆ is Hydrogen, CHO, CH₃, COCH₃, CH═CH₂, CH═CH(CH₃), (CH═CH)₂CH₃, (CH═CH)₂CH═CH₂, (CH═CH)₃CH₃, COCH═CH₂, COCH═CHCH₃, or CO(CH₂)₃CH₃.

In one embodiment, protonophore is 2-(but-3-en-2-one), 3-hydroxy, 5,7-dimethyl, chromone, 2-(prop-2-en-1-one), 3-hydroxy, 6,7-dimethyl, chromone, 2-(2-methyl-prop-2-en-1-one), 3-hydroxy, 7-methyl, chromone, 2-acetyl, 3-hydroxy, 5,7-dimethyl, 6-ethenyl, chromone, 3-hydroxy, 6-(propen-1-yl),7-(but-2-en-1-one), chromone, 2((1E)-buta-1,3-dien-1-yl), 3-hydroxy, 5,6-dimethyl, 8-acetyl,chromone, 2-(prop-2-en-1-one), 3-hydroxy, 6-(propen-1-yl), chromone, 2-(but-2-en-1-one), 3-hydroxy, 6-ethenyl, chromone, 2,5-dimethyl, 6-((2E)-but-2-en-2-yl), 8-acetyl, chromone, 2,3,5-trimethyl, 6,8-diformyl, 7-hydroxy, chromone, 2,3-dimethyl, 6-(prop-2-en-1-one), 7-hydroxy, 8-acetyl, chromone, 2,3,5-trimethyl, 6,8-diformyl, 7-hydroxy, chromone, 2,3-dimethyl, 6-(prop-2-en-1-one), 7-hydroxy, 8-acetyl, chromone, 2-(propen-1-yl), 3-methyl, 6,8-diacetyl, 7-hydroxy, chromone, 2-(propen-1-yl), 6-acetyl, 7-hydroxy, 8-ethenyl, chromone, 2,3-dimethyl, 6-ethenyl, 7-hydroxy, 8-formyl, chromone, 6-(prop-2-en-1-one), 7-hydroxy, 8-ethenyl, chromone, 2,3-dimethyl, 6-formyl, 7-hydroxy, 8-ethenyl, chromone, 2,8-diethenyl, 3-methyl, 6-acetyl, 7-hydroxy, chromone, 3,6-diethenyl, 7-hydroxy, 8-formyl, chromone, 2,3-diethenyl, 6-acetyl, 7-hydroxy, 8-methyl,chromone, 3,8-diethenyl, 6-formyl, 7-hydroxy, chromone, 3-methyl, 7-hydroxy, 8-(but-2-en-1-one), chromone, 2((1E)-buta-1,3-dien-1-yl), 3-methyl, 6-acetyl, 7-hydroxy, chromone, 3-methyl, 6-(propen-1-yl), 7-hydroxy, 8-acetyl, chromone, 2((1E)-buta-1,3-dien-1-yl), 3-methyl, 7-hydroxy, 8-acetyl, chromone, 3-methyl, 6-(butan-1-one), 7-hydroxy, chromone, 6-acetyl, 7-hydroxy, 8-((1E,3E)-penta-1,3-dien-1-yl), chromone, 2-ethenyl, 3,7-dihydroxy, 6-(prop-2-en-1-one), 8-methyl, chromone, 2-(2-methylprop-1-en-1-yl), 3,7-dihydroxy, 6-acetyl, 8-methyl, chromone, 2,6,8-triethenyl, 3,7-dihydroxy, chromone, 2,6-di-(propen-1-yl), 3,7-dihydroxy, chromone, 2,3,5-trimethyl, 6,8-diformyl, 7-hydroxy, dihydrochromone, 6-((2E,4E)-hexa-2,4-dien-1-one), 7-hydroxy, 8-acetyl, dihydrochromone, 6-formyl, 7-hydroxy, 8-((1E,3E,5E)-hexa-1,3,5-trien-1-yl), dihydrochromone, 6-(prop-2-en-1-one), 7-hydroxy, 8-(propen-1-yl), dihydrochromone, 6-((1E,3E,5E)-hexa-1,3,5-trien-1-yl), 7-hydroxy, 8-acetyl, dihydrochromone, 6-(but-2-en-1-one), 7-hydroxy, 8-(prop-2-en-1-one), dihydrochromone, 6-formyl, 7-hydroxy, 8-(pentan-1-one), dihydrochromone, 3,6-dihydroxy-9-oxoxanthene-4,5-dicarbaldehyde, 4,7-diacetyl-3,6-dihydroxy-2-methylxanthen-9-one, 2-acetyl, 3-sulfanyl, 6-((1E)-buta-1,3-dien-1-yl), chromone, 2-(prop-2-en-1-one), 3-sulfanyl, 6-methyl, chromone, 2-methyl, 3-sulfanyl, 7-(pentan-1-one), chromone, 2,3-diethenyl, 6-formyl, 7-sulfanyl, 8-methyl, chromone, 2-ethenyl, 5,8-dimethyl, 6-formyl, 7-sulfanyl, chromone, 2-ethenyl, 5,8-dimethyl, 6-formyl, 7-sulfanyl, chromone, 2-(propen-1-yl), 3-ethenyl, 6-methyl, 7-sulfanyl, 8-acetyl, chromone, 2-(propen-1-yl), 3-ethenyl, 5-methyl, 6,8-diformyl, 7-sulfanyl, chromone, 6-formyl, 7-sulfanyl, 8-((1E,3E,5E)-hepta-1,3,5-trien-1-yl), dihydrochromone, 6-((1E,3E,5E)-hepta-1,3,5-trien-1-yl), 7-sulfanyl, 8-formyl dihydrochromone, 6-(pentan-1-one), 7-sulfanyl, 8-methyl, dihydrochromone, 6-methyl, 7-sulfanyl, 8-(pentan-1-one), dihydrochromone, 2,3,8-trimethyl, 5,7-diformyl, 6-hydroxy, 1,4-naphtoquinone, 2,3-di-(propen-1-yl), 6-hydroxy, 7-acetyl, 1,4-naphtoquinone, 2,3,5,8-tetramethyl, 6-hydroxy, 7-acetyl, 1,4-naphtoquinone, 2,5-dimethyl, 3-(propen-1-yl), 6-hydroxy, 7-acetyl, 1,4-naphtoquinone, 2-(propen-1-yl), 3,5-dimethyl, 6-hydroxy, 7-acetyl, 1,4-naphtoquinone, 2,3,7,8-tetramethyl, 5-acetyl, 6-hydroxy, 1,4-naphtoquinone, 2,3,8-triethenyl, 5-acetyl, 6-hydroxy, 1,4-naphtoquinone, 2,3-diethenyl, 5,7-diacetyl, 6-hydroxy, 8-methyl, 1,4-naphtoquinone, 2,8-diethenyl, 5,7-diformyl, 6-hydroxy, 1,4-naphtoquinone, 2-(propen-1-yl), 5,7-acetyl, 6-hydroxy, 8-ethenyl, 1,4-naphtoquinone, 1,3-diacetyl, 2-hydroxy, anthraquinone,1,3-formyl, 2-hydroxy, anthraquinone, 2,6-dihydroxy, 3,7-diformyl, anthraquinone, 2,6-dihydroxy, 1,5-diformyl, anthraquinone,2,3,5,8-tetramethyl, 6-sulfanyl, 7-formyl, 1,4-naphtoquinone, 2-((1E,3E,5E)-hepta-1,3,5-trien-1-yl), 6-sulfanyl, 7-acetyl, 1,4-naphtoquinone, 2-((1E,3E,5E)-hepta-1,3,5-trien-1-yl), 6-sulfanyl, 7-formyl, 1,4-naphtoquinone, 2-(propen-1-yl), 6-sulfanyl, 7-(but-2-en-1-one), 1,4-naphtoquinone,2,3-dimethyl, 6-sulfanyl, 7-ethenyl, 1,4-naphtoquinone, 2-(propen-1-yl), 6-sulfanyl, 7-ethenyl, 1,4-naphtoquinone, 2-ethenyl, 6-sulfanyl, 7-(propen-1-yl), 1,4-naphtoquinone, 3-ethenyl, 6-sulfanyl, 7-(propen-1-yl), 1,4-naphtoquinone, 3-(propen-1-yl), 6-sulfanyl, 7-ethenyl, 1,4-naphtoquinone, 2-((1E,3E,5E)-hexa-1,3,5-trien-1-yl), 6-sulfanyl, 1,4-naphtoquinone, 6-sulfanyl, 7-(hexan-1-one), 1,4-naphtoquinone, 2-sulfanyl, anthracene-9,10-dione, 3-hydroxy, 6,7-dimethyl, chromenylium, 3-hydroxy, 2,6,7-trimethyl, chromenylium, 3-hydroxy, 6-ethenyl, chromenylium, 2-methyl, 3-hydroxy, 6-ethenyl, chromenylium, 3-hydroxy, 7-ethenyl, chromenylium, 2-methyl, 3-hydroxy, 7-ethenyl, chromenylium, 2-(propen-1-yl), 4-hydroxy, chromenylium,4-hydroxy, 7-ethenyl, chromenylium,7-ethenyl, 8-hydroxy, chromenylium, 2-methyl, 7-ethenyl, 8-hydroxy, chromenylium, 3,6-dihydroxy, 5-methyl, 7-ethenyl, chromenylium, 3,6-dihydroxy, 5,7,8-trimethyl, chromenylium, 2-methyl, 3,6-dihydroxy, 7-(propen-1-yl), chromenylium, 2,4,7-triamine, 3,5,6,8-tetraethenyl, quinoline, 2-N,4-N,7-N-trimethyl, 2,4,7-triamine, 3,5,6,8-tetramethyl, quinoline, 2,5,8-triamine, 3,4,7-trimethyl, 6-((1E)-buta-1,3-dien-1-yl), isoquinoline, N-5-methyl, 2,5,8-triamine, 3,7-dimethyl, 4,6-diethenyl, isoquinoline, N-2,N-8-methyl, 2,5,8-triamine, 3,4,6,7-tetramethyl, isoquinoline, 5,7-diethenyl-6-methyl-1H-naphtho[2,3-b]pyrrole-9-Carbaldehyde, 6,8-dimethyl, 7-((1E,3E)-penta-1,3-dien-1-yl), 1-H-quinolin-4-one, or 6,8-dimethyl, 7-((1E,3E)-penta-1,3-dien-1-yl), 1-H-quinolin-4-one.

In one embodiment, the protonophore of Formula (I) is represented by a protonophore of Formula (IV)

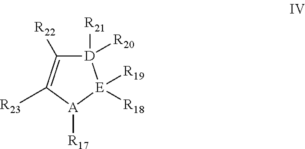

wherein: A is O, S, C, or N; D and E are each independently C or N; R₁₇ is Absent, ═O, or Hydrogen; R₁₈ is Absent, Hydrogen, CHO, OH, SH, CH₃, COCH₃, CH═CH₂, CH═CH(CH₃), CH═C(CH₃)₂, (CH═CH)₂CH₃, (CH═CH)₃CH₃, COCH═CH₂, COCH═CHCH₃, CO(CH₂)₃CH₃, CO(CH₂)₇CH₃, or CO-phenyl; R₁₉ and R₂₀ are each independently Absent or Hydrogen; R₂₀ is Absent, Hydrogen, OH, SH, CHO, COCH₃, CH₃, C(CH₃)₃, COCH═CH₂, COCH═CH(CH₃); CO(CH₂)₃CH₃, or CO(CH₂)₅CH₃; and R₂₂ is Hydrogen, CH₃, C(CH₃)₃, CH═CH₂, CH═CH(CH₃), CH═C(CH₃)₂, CH═CHCH═CH₂, (CH═CH)₂CH₃, (CH═CH)₃CH₃, or COCH═CH₂; provided that: when A is O or S, then R₁₇ is Absent, when A is C, then R₁₇ is ═O, and when A is N, then R₁₇ is Hydrogen.

In one embodiment, the protonophore is 2-hydroxy, 3-acetyl, 4,5-di-(propen-1-yl), furan, 2-hydroxy, 3-(prop-2-en-1-one), 4,5-diethenyl, furan, 2,4-di-(prop-2-en-1-one), 3-hydroxy, 5-ethenyl, furan, 2-hydroxy, 3,5-diformyl, 4-[(1E)-buta-1,3-dien-1-yl], thiofuran, 2-hydroxy, 3-acetyl, 4-methyl, 5-ethenyl, thiofuran, 2-hydroxy, 3,5-acetyl, 4-[(1E,3E)-penta-1,3-dien-1-yl], thiofuran, 2-sulfanyl, 3-formyl, 4,5-di(propen-1-yl), furan, 2-sulfanyl, 3-(but-2-en-1-one), 4,5-diethenyl, furan, 2-sulfanyl, 3-(pentan-1-one), 4,5-dimethyl, furan, 2-methyl, 3-sulfanyl, 4-[(1E,3E,5E)-hepta-1,3,5-trien-1-yl], 5-formyl, furan, 2-methyl, 3-sulfanyl, 5-((2E,4E,6E)-octa-2,4,6-trien-1-one), furan, 3-sulfanyl, 5-(heptan-1-one), furan, 2,3-dithiol, 4-tert-butyl, 5-methyl, furan, 2,7-diacetyl, 3-hydroxy, 6-((1E,3E)-penta-1,3-dien-1-yl), benzofuran, 2-(prop-2-en-1-one), 3-hydroxy, 5-methyl, benzofuran, 2-acetyl, 3-hydroxy, 5-ethenyl, 6-methyl, benzofuran, 2-(but-2-en-1-one), 3-hydroxy, benzofuran, 2-acetyl, 3-hydroxy, 5-(propen-1-yl) benzofuran, 2,5-di-(prop-2-en-1-one), 3-hydroxy, benzofuran, 2-(but-2-en-1-one), 3-hydroxy, 5-acetyl, 6-methyl, benzofuran, 2-acetyl, 3-hydroxy, 5-(but-2-en-1-one), 6-methyl, benzofuran, 2-acetyl, 3-hydroxy, 5-(2-methylprop-1-en-1-yl), benzofuran, 2-acetyl, 3-hydroxy, 5,6-dimethyl, benzofuran, 2,6-di(propen-1-yl), 3-hydroxy, 5,7-diacetyl, benzofuran, 2-acetyl, 3-hydroxy, 7-(pentan-1-one), benzofuran, 2-(pentan-1-one), 3-hydroxy, 7-formyl, benzofuran, 2-formyl, 3-hydroxy, 5-(pentan-1-one), benzofuran, 2-(pentan-1-one), 3-hydroxy, 5-acetyl, benzofuran, 2,5-diethenyl, 3,7-dihydroxy, 4-formyl, 6-methyl, benzofuran, 2-(2-methylprop-1-en-1-yl), 3,7-dihydroxy, 4-acetyl, 6-ethenyl, benzofuran, 3,7-dihydroxy, 4-(but-2-en-1-one), 6-ethenyl, benzofuran, 2-(2-methylprop-1-en-1-yl), 3,7-dihydroxy, 6-acetyl, benzofuran, 2,4-diethenyl, 3,7-dihydroxy, 6-acetyl, benzofuran, 2-((1E,3E)-penta-1,3-dien-1-yl), 3-sulfanyl, 4,7-diformyl, benzofuran, 2-(propen-1-yl), 3-sulfanyl, 4,7-diformyl, 6-methyl, benzofuran, 2,5,6-trimethyl, 3-sulfanyl, 7-formyl, benzofuran, 2,7-dimethyl, 4,6-diformyl, 5-hydroxy, inden-1-one, 3,7-dimethyl, 4,6-diformyl, 5-hydroxy, inden-1-one, 4,6-diformyl, 5-hydroxy, 7-ethenyl, inden-1-one, 4-acetyl, 5-hydroxy, 6-(butan-1-one), inden-1-one, 4-formyl, 5-hydroxy, 6-(but-2-en-1-one), dihydro-inden-1-one, 4-acetyl, 5-hydroxy, 6-(but-2-en-1-one), dihydro-inden-1-one, 2-(propen-1-yl), 4-methyl, 5-sulfanyl, 6-formyl, inden-1-one, 2-(propen-1-yl), 3-methyl, 5-sulfanyl, inden-1-one, 2-ethenyl, 5-sulfanyl, 6-methyl, inden-1-one, 2,4-dimethyl, 5-sulfanyl, 6-(prop-2-en-1-one), inden-1-one, 2-((1E,3E,5E)-hepta-1,3,5-trien-1-yl), 4,6-diacetyl, 5-sulfanyl, inden-1-one, 4-acetyl, 5-sulfanyl, 6-(hexan-1-one), inden-1-one, 5-sulfanyl, 6-(pentan-1-one), inden-1-one, 4,7-diethenyl, 5-sulfanyl, 6-formyl, dihydro-inden-1-one, 5-sulfanyl, 6-(propen-1-yl), dihydro-inden-1-one, 4-formyl, 5-sulfanyl, 6-((1E,3E)-penta-1,3-dien-1-yl), dihydro-inden-1-one, 4-(pentan-1-one), 5-sulfanyl, dihydro-inden-1-one, 3,6-dihydroxy-9-oxofluorene-2,7-dicarbaldehyde, 3,6-dihydroxy-9-oxofluorene-4,5-dicarbaldehyde, 3,6-dihydroxy-9-oxofluorene-2,5-dicarbaldehyde, 2-tert-butyl, 4-(2-methyl-propen-1-yl), 5-hydroxy, oxazole, 2-(nonan-1-one), 4-methy, 5-hydroxy, oxazole, 2-benzoyl, 4-(2-methylprop-1-en-1-yl), 5-hydroxy, oxazole, 3-tert-butyl, 4-(propen-1-yl), 5-hydroxy, isoxazole, 3-(heptan-1-one), 4-methyl, 5-hydroxy, isoxazole, or 5,7-diethenyl-6-methyl-1H-naphtho[2,3-b]pyrrole-9-carbaldehyde.

In one embodiment, the protonophore of Formula (I) is represented by a protonophore of Formula (V)

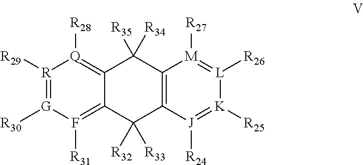

wherein: G, K, L, Q, and R are each independently C; F is O; J. And M are each independently C, O; R₂₄ and R₂₇ are each independently Absent or CH₃; R₂₅ and R₂₆ are each independently CH₃ or OH; R₂₈ and R₃₀ are each independently CH₃; R₂₉ is OH; R₃₁ is Absent; and R₃₂, R₃₃, R₃₄, R₃₅ are each independently Hydrogen, wherein the contacting is in vitro, in vivo, or directly on an organism.

In one embodiment, the protonophore is 3,7-dihydroxy-2,4,6,8-tetramethyl-5H,10H-pyrano[3,2-g]chromene-1,9-bis(ylium) or 3,8-dihydroxy-2,4,7,9-tetramethyl-5H,10H-pyrano[2,3-g]chromene-1,6-bis(ylium).

The present invention provides a pharmaceutical composition for treating a disorder, disease, or condition benefiting from a protonophore-induced uncoupling of mitochondrial oxidative phosphorylation in a patient in need thereof including a protonophore of Formula (I) as defined herein, or a protonophore of Formula (II) as defined herein, or a protonophore of Formula (III) as defined herein, or a protonophore of Formula (IV) as defined herein, or a protonophore of Formula (V) as defined herein, and a pharmaceutically acceptable diluent or carrier.

The present invention provides a method of inhibiting or killing a fungus. The method includes contacting the fungus with an effective anti-fungal amount of a protonophore of Formula (I) as defined herein, or a protonophore of Formula (II) as defined herein, or a protonophore of Formula (III) as defined herein, or a protonophore of Formula (IV) as defined herein, or a protonophore of Formula (V) as defined herein, wherein the contacting is in vitro, in vivo, or directly on the fungus.

In one embodiment, the method of inhibiting or killing a fungus is used to preserve wood.

The present invention provides a method of inhibiting or killing a bacterium. The method includes contacting the bacterium with an effective anti-bacterial amount of a protonophore of Formula (I) as defined herein, or a protonophore of Formula (II) as defined herein, or a protonophore of Formula (III) as defined herein, or a protonophore of Formula (IV) as defined herein, or a protonophore of Formula (V) as defined herein, wherein the contacting is in vitro, in vivo, or directly on the bacterium.

In one embodiment, the bacterium includes bacteria of oral plaque, Helicobacter pylori, or a combination thereof.

The present invention provides a method of inhibiting or killing an antibiotic resistant bacterium. The method includes: contacting the bacterium with an effective anti-bacterial amount of a Formula (I) as defined herein, or a protonophore of Formula (II) as defined herein, or a protonophore of Formula (III) as defined herein, or a protonophore of Formula (IV) as defined herein, or a protonophore of Formula (V) as defined herein, wherein the contacting is in vitro, in vivo, or directly on the antibiotic resistant bacterium.

The present invention provides a method of inhibiting or killing a plant. The method includes contacting the plant with an effective anti-plant amount of a protonophore of Formula (I) as defined herein, or a protonophore of Formula (II) as defined herein, or a protonophore of Formula (III) as defined herein, or a protonophore of Formula (IV) as defined herein, or a protonophore of Formula (V) as defined herein, wherein the contacting is in vitro, in vivo, or directly on the plant.

The present invention provides a method of inhibiting or killing a weed. The method includes contacting the weed with an effective anti-weed amount of a protonophore Formula (I) as defined herein, or a protonophore of Formula (II) as defined herein, or a protonophore of Formula (III) as defined herein, or a protonophore of Formula (IV) as defined herein, or a protonophore of Formula (V) as defined herein, wherein the contacting is in vitro, in vivo, or directly on the weed.

The present invention provides a method of inhibiting or killing an insect. The method includes contacting the insect with an effective anti-insect amount of a protonophore of Formula (I) as defined herein, or a protonophore of Formula (II) as defined herein, or a protonophore of Formula (III) as defined herein, or a protonophore of Formula (IV) as defined herein, or a protonophore of Formula (V) as defined herein, wherein the contacting is in vitro, in vivo, or directly on the insect.

The present invention provides a method of inhibiting or killing a pest. The method includes contacting the insect with an effective anti-insect amount of a protonophore of Formula (I) as defined herein, or a protonophore of Formula (II) as defined herein, or a protonophore of Formula (III) as defined herein, or a protonophore of Formula (IV) as defined herein, or a protonophore of Formula (V) as defined herein, wherein the contacting is in vitro, in vivo, or directly on the pest.

The present invention provides a method of inhibiting or killing a cancer. The method includes contacting the cancer with an effective anti-cancer amount of a protonophore of Formula (I) as defined herein, or a protonophore of Formula (II) as defined herein, or a protonophore of Formula (III) as defined herein, or a protonophore of Formula (IV) as defined herein, or a protonophore of Formula (V) as defined herein, wherein the contacting is in vitro, in vivo, or directly on an organism.

The present invention provides a compound including a protonophore of Formula (I) as defined herein, or a protonophore of Formula (II) as defined herein, or a protonophore of Formula (III) as defined herein, or a protonophore of Formula (IV) as defined herein, or a protonophore of Formula (V) as defined herein, for use in medical therapy.

The present invention provides a use of a compound including a protonophore of Formula (I) as defined herein, or a protonophore of Formula (II) as defined herein, or a protonophore of Formula (III) as defined herein, or a protonophore of Formula (IV) as defined herein, or a protonophore of Formula (V) as defined herein, to prepare a medicament for treatment of a disorder, disease, or condition benefiting from an uncoupling of mitochondrial respiration in a patient in need.

On one embodiment, the medicament includes a pharmaceutically acceptable diluent or carrier.

The present invention provides a compound including a protonophore of Formula (I) as defined herein, or a protonophore of Formula (II) as defined herein, or a protonophore of Formula (III) as defined herein, or a protonophore of Formula (IV) as defined herein, or a protonophore of Formula (V) as defined herein, for use in medical therapy.

The present invention provides the use of a compound including a protonophore of Formula (I) as defined herein, or a protonophore of Formula (II) as defined herein, or a protonophore of Formula (III) as defined herein, or a protonophore of Formula (IV) as defined herein, or a protonophore of Formula (V) as defined herein, to prepare a medicament for treatment of a disorder, disease, or condition benefiting from an uncoupling of mitochondrial respiration in a patient in need.

In one embodiment, the medicament includes a pharmaceutically acceptable diluent or carrier.

### BRIEF DESCRIPTION OF THE DRAWINGS

Embodiments of the invention may be best understood by referring to the following description and accompanying drawings, which illustrate such embodiments. In the drawings:

FIG. 1 illustrates a general mechanism of uncoupling by lipophilic weak acids.

FIG. 2 illustrates thermodynamic considerations of uncoupling by proton shuttles.

FIG. 3 illustrates the proposed resistance-lowering effect of asymmetric molecular distribution.

FIG. 4 illustrates contributions of partitioning behavior and of molecular size and shape to the estimation of permeability/resistance to diffusion.

FIG. 5 illustrates a plot of the interaction of pKa and lipophilicity.

FIG. 6 illustrates thermodynamic considerations for multi-protic proton shuttles.

FIG. 7 illustrates special cases of the proton shuttle mechanism: cations and weak lipophilic bases.

FIG. 8 illustrates a special case of the proton shuttle mechanism: compounds capable of overcoming insufficient lipophilicity through oligomerization.

FIG. 9 illustrates the relationship between predicted and measured activity for a 48-compound test set.

FIG. 10 illustrates naturally-occurring chemical templates conducive to proton shuttle activity and examples of optimized derivatives.

FIG. 11 illustrates optimized di-protic derivatives with greater predicted activity than their mono-protic counterparts.

FIG. 12 illustrates an evaluation of the potential circuits of a di-protic compound.

FIG. 13 illustrates adapting the model to predict the activity of cationic compounds.

FIG. 14 illustrates adapting the model to predict the activity of basic compounds.

FIG. 15 illustrates an example of proton shuttle exhibiting trans-cis photoisomerization.

FIG. 16 illustrates halochromic proton shuttles designed for the direct assessment of molecular distribution.

FIG. 17 illustrates the enhancement of skeletal muscle cell basal glucose uptake and suppression of hepatocyte glucose-6-phosphatase (G6Pase) activity by the reference weak uncoupler 2,4-dinitrophenol.

FIG. 18 illustrates chemical structures of 50 naturally-occurring phenolic compounds screened for uncoupling activity.

FIG. 19 illustrates representative oxygen consumption tracings from isolated mitochondria illustrating the instantaneous increase in the rate of basal oxygen consumption (state 4 respiration; non-ADP-stimulated) characteristic of uncoupling activity.

FIG. 20 illustrates the relationship between uncoupling in isolated mitochondria and up regulation of glucose uptake in skeletal muscle cells and the complete glucose uptake dataset for compounds of interest and their respective nonionizable class parent compound devoid of uncoupling activity, sorted by class.

FIG. 21 illustrates the powerful insulin-like and metformin-like suppression of G6Pase activity in H4IIE hepatocytes induced by uncouplers of the 4′ hydroxychalconoid family.

FIG. 22 illustrates the uncoupling activity of 2,4-dinitrophenol and 50 screened compounds plotted against compound acid-dissociation behavior (pKa(1)) and lipophilicity (log P_{octanol-water} value of the protonated species), two main determinants of uncoupling activity.

FIG. 23 illustrates the core structures conferring activity to identified uncouplers.

FIG. 24 illustrates the concentration-activity relationship of 2,4-dinitrophenol and of fourteen compounds of the screening test set exhibiting greatest uncoupling activity at 100 μM.

FIG. 25 illustrates the proposed distinction between inhibition and uncoupling of oxidative phosphorylation as mechanisms for perturbing energy homeostasis for the indirect activation of AMPK.

FIG. 26 is a block diagram illustrating an exemplary computer-assisted method of generating a protonophore

FIG. 27 is a block diagram illustrating an exemplary method of designing the protonophore.

The drawings are not necessarily to scale. Like numbers used in the figures refer to like components, steps, and the like. However, it will be understood that the use of a number to refer to a component in a given figure is not intended to limit the component in another figure labeled with the same number.

### DETAILED DESCRIPTION OF THE INVENTION

The present invention provides a computer-assisted method of generating a protonophore, the method requiring the use of a computer including a processor. The method includes: designing the protonophore; calculating, using the processor, an estimated protonophoric activity across a biological membrane with a pH gradient for the protonophore; producing the protonophore if the estimated protonophoric activity across the biological membrane with the pH gradient for the protonophore corresponds to an U₅₀ of about 20 μM or less; and determining the uncoupling activity of the protonophore. The present invention also provides novel protonophores that meet the above requirement and their methods of use.

Simple thermodynamic model of unassisted proton shuttle uncoupling and prediction of activity from calculated speciation, lipophilicity, and molecular geometry (Martineau, Journal of Theoretical Biology, 303: 33-61; 2012)

Abstract: A mechanistic model of uncoupling of oxidative phosphorylation by lipophilic weak acids (i.e., proton shuttles) was developed for the purposes of predicting the relative activity of xenobiotics of widely varying structure and of guiding the design of optimized derivatives. The model is based on thermodynamic premises not formulated elsewhere that allow for the calculation of steady-state conditions and of rate of energy dissipation on the basis of acid-dissociation and permeability behavior, the later estimated from partitioning behavior and geometric considerations. Moreover, permeability of either the neutral or of the ionized species is proposed to be effectively enhanced under conditions of asymmetrical molecular distribution. Finally, special considerations were developed to accommodate multi-protic compounds. The comparison of predicted to measured activity for a diverse test set of 48 compounds of natural origin spanning a wide range of activity yielded a Spearman's rho of 0.90. The model was used to tentatively identify several novel proton shuttles, as well as to elucidate core structures particularly conducive to proton shuttle activity from which optimized derivatives can be designed. Principles of design were formulated and examples of derivatives projected to be active at concentrations on the order of 10⁻⁷ M are proposed. Among these are di-protic compounds predicted to shuttle two protons per cycle iteration and proposed to maximally exploit the proton shuttle mechanism. This work promotes the design of highly active, yet easily-metabolized (i.e., metabolically-unstable) uncouplers for therapeutic applications, namely the indirect activation of AMP-kinase, as well as for various industrial applications where low persistence is desirable.

1. Introduction

Oxidative phosphorylation is said to be less than maximally coupled when the proton motive force across the mitochondrial inner membrane is dissipated rather than harnessed for the resynthesis of ATP. In general, mitochondria do not operate at maximal transduction efficiency. Instead, there is a constitutive proton leak across the proton-impermeable inner membrane, mediated largely by the adenine nucleotide translocase [1A], that can be augmented under a variety of physiological situations by non-esterified fatty acids working in concert with members of the uncoupling protein family within the inner membrane [2A]. This partial dissipation of the proton motive force serves to reduce the production of reactive oxygen species by the electron transport chain under conditions of low respiratory flux and high transmembrane potential, is responsible for the non-shivering thermogenesis of brown adipocytes and cold-adapted skeletal muscle, and is necessary for some anabolic and catabolic functions [2A]. It may also confer sensitivity to nutrient-sensing cells such as pancreatic beta cells [3A].

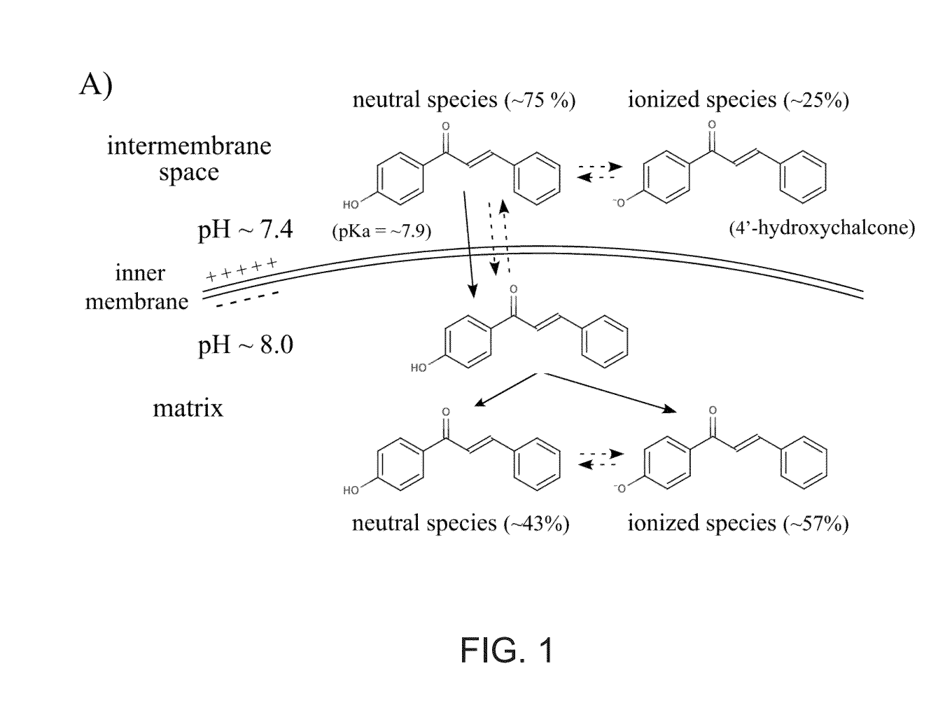

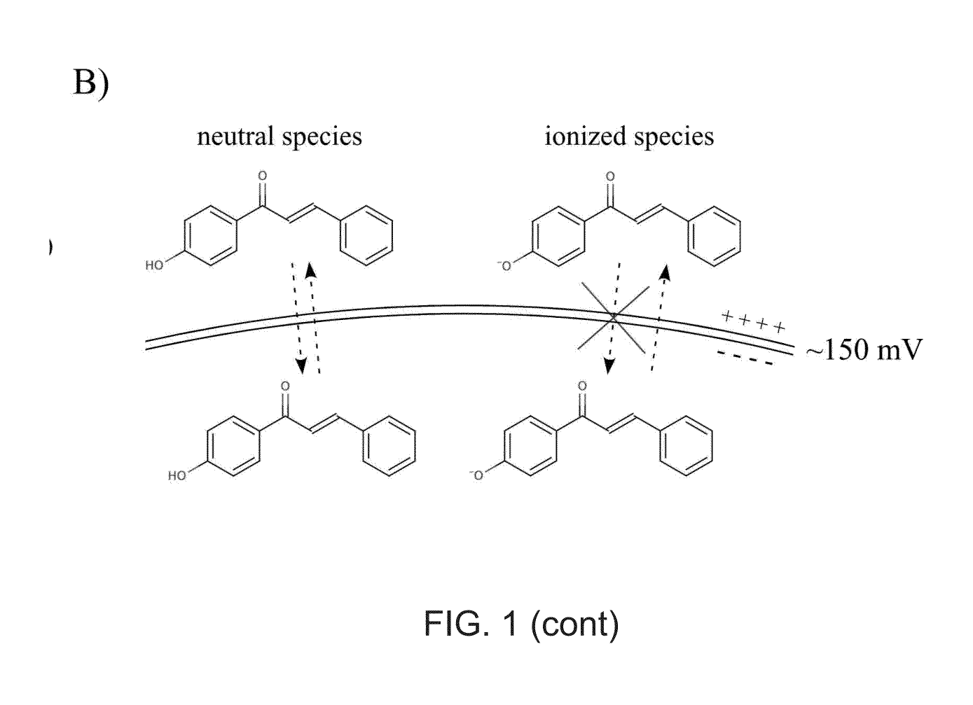

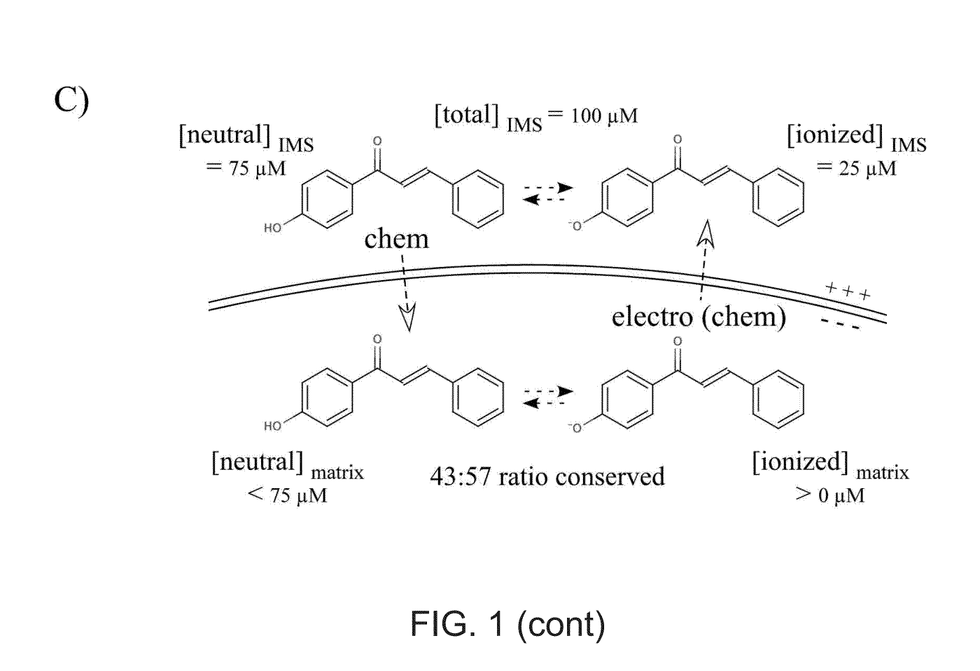

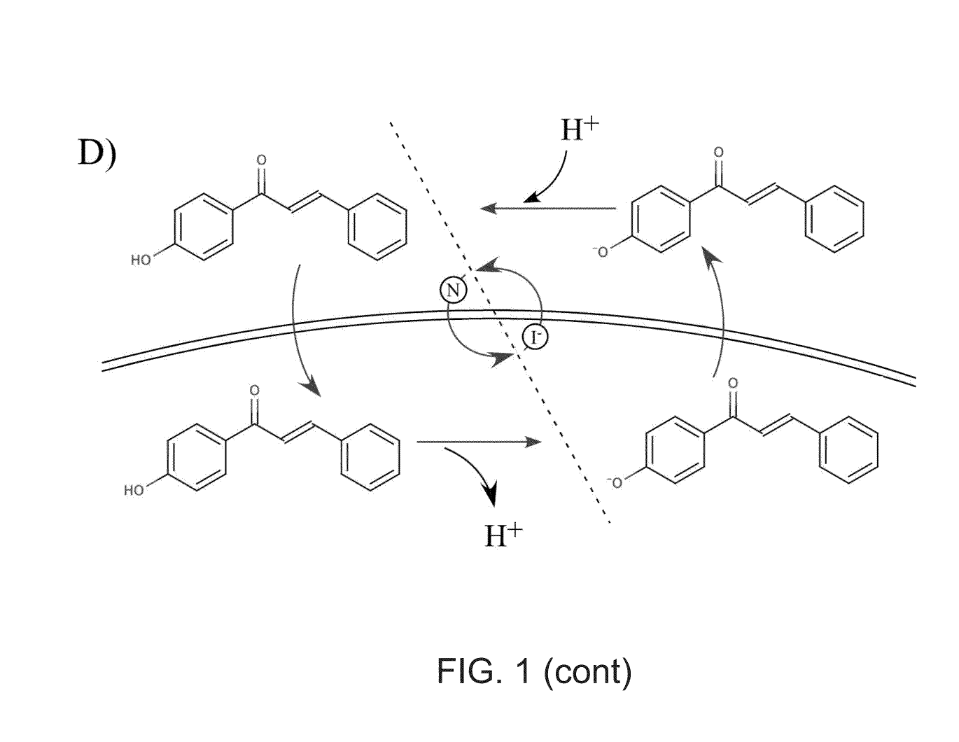

In the larger sense, however, dissipation of the proton motive force, or uncoupling of respiration from phosphorylation, is a state of decreased metabolic efficiency that can lead to ATP starvation and that is induced by xenobiotics rather than physiologically-regulated. Compounds capable of inducing this state are termed uncouplers. Xenobiotic-induced uncoupling can be mediated through a variety of cationophoric mechanisms [2A, 4A]. The term uncoupler, however, is normally reserved for compounds that specifically exhibit protonophoric activity. The first description of such compounds dates back more than 60 years [5A]. Uncouplers are small ionizable compounds characterized by moderate to high lipophilicity and extensive charge delocalization. They act through a simple cyclical mechanism, summarized in FIG. 1. Elucidation of the broad strokes of this mechanism was key to the formulation of the chemiosmotic theory of energy transduction [6A], and the mechanism has since received considerable attention (for reviews, see [2A, 4A, 7A-9A]).

It is important to distinguish between two types of uncouplers. The first are unassisted proton shuttle uncouplers or classical uncouplers (referred to henceforth as proton shuttles), whose activity is completely attributable to the Mitchellian mechanism of uncoupling, described above. The activity of such compounds is directly proportional to concentration, and the most potent among these can induce complete uncoupling (i.e., point at which the induced futile cycle makes use of all the respiratory capacity normally available for ATP resynthesis) at low micromolar concentrations. The second type are protein-assisted uncouplers, whose Mitchellian proton shuttle activity is augmented by incompletely understood interactions with protein constituents of the mitochondrial inner membrane, such as the adenine nucleotide translocase, that may resemble the interaction between fatty acids and proteins of the uncoupling family [2A, 4A, 10A]. The most potent protein-assisted uncouplers, man-made compounds used as industrial biocides [9A] and typically characterized by presence of difficult-to-metabolize (i.e., metabolically-stable) chemical groups, can induce complete uncoupling at concentrations as low as 10-100 nM [2A, 4A]. Evidence for protein interaction includes sensitivity to inhibitors such as carboxyatractylate or 6-ketocholestanol [2A, 10A], greater potency in mitochondrial preparations than in protein-free systems [2A, 11A], the tentative identification of protein partners by photoaffinity labeling [2A], and departure of the dose-response relationship from the 1:1 relationship predicted of the Mitchellian mechanism [2A, 10A].

The activity of proton shuttles is conferred simply by physicochemical properties and a three-dimensional structure that are conducive to efficient transmembrane diffusion. Therefore, there are few structural constraints to activity, and, consequently, proton shuttles comprise a vast chemical space of naturally-occurring and man-made compounds. Interestingly, a large number of non-nitrogenous phenolic plant secondary metabolites exhibit this activity [12A], possibly contributing to thermogenesis or to defense against predatory organisms. While protein-assisted uncouplers share with proton shuttles the same fundamental physicochemical properties, interaction with a protein partner necessarily imposes more stringent, key-in-lock type structural constraints on activity. Moreover, the properties that confer a high quality of interaction with the target protein may be at odds with those that confer efficient transmembrane diffusion. Thus, attempts to relate physicochemical properties to uncoupling activity in a class-independent fashion, especially those that rely entirely on a mathematical best-fit approach, may be confounded by failure to distinguish between proton shuttles and protein-assisted uncouplers, possibly even resulting in physiologically-unsound conclusions. In this same vein, conclusions derived from structure-activity relationship studies restricted to a single class of protein-assisted uncouplers cannot be expected to be universal.

Although uncouplers are best known for industrial uses and are notorious for chemical persistence, these compounds also have potential therapeutic applications. Most promising among these is the use of short-lived/easily-metabolized uncouplers for acutely perturbing energy homeostasis to indirectly stimulate the AMP-activated protein kinase (AMPK) signaling pathway, a key target for insulin resistance and associated metabolic diseases [13A-15A]. Along this line, our group has shown that uncoupling-induced activation of AMPK is often the basis of the insulin-like effects of plant-derived medicinal products [16A, 17A], and we have recently demonstrated insulin-like activities of remarkable magnitude induced by several well-tolerated novel uncouplers of natural origin [12A, 18A]. Another potential therapeutic application is the use of non-orally bioavailable uncouplers as anti-bacterial agents. Due to a largely unconstrained structure-activity relationship and a clear non-dependency on difficult-to-metabolize groups, proton shuttles are better suited to such applications than protein assisted uncouplers. For these same reasons, proton shuttles may also be well suited to industrial biocidal applications where chemical persistence or bioavailability is undesirable. While the maximal activity of proton shuttles is clearly lower than that of protein-assisted uncouplers, it is likely that the full potential of the proton shuttle mechanism has yet to be exploited.

In light of such potential applications, a comprehensive model is needed to guide the design of novel compounds that maximally exploit the proton shuttle mechanism and to facilitate the identification of proton shuttle activity in known compounds. This is of relevance also to the toxicological screening of compound databases. However, a barrier to such a model is that understanding of proton shuttle uncoupling is incomplete. The impetus for the present work was therefore to provide new insight into this mechanism, in particular into the contribution of physicochemical parameters to activity and the interaction among them, in order to develop a tool with broader predictive powers than currently available regression models. While focus of the present work is on compounds composed exclusively of C, H, and O, presumably easily-metabolized and non-persistent, the theoretical framework developed here and the design principles extended from this framework are proposed to apply to all proton shuttles.

2. Methods

2.1 Estimation of Physicochemical Parameters

All physicochemical parameters used for the prediction of proton shuttle activity were calculated using the Marvin chemoinformatics suite (versions 5.2 and 5.3; academic package; ChemAxon Kft., Budapest, Hungary). Structures verified for accuracy against entries in the PubChem. Compound database (National Center for Biotechnology Information; http://pubchem.Ncbi.Nlm.Nih.Gov) or the ChemSpider compound database (Royal Society of Chemistry; http://www.Chempider.Com) were manually drawn in MarvinSketch. The Protonation calculator (v. 5.2) was used to estimate acid-dissociation constants (pKa) and patterns of speciation at pH 7.4 (taken to correspond to the pH of the mitochondrial inter-membrane space) and pH 8.0 (taken to correspond to the pH of the mitochondrial matrix). The accuracy of this substructure-based algorithm has recently been validated [19A]. The Partitioning calculator (v. 5.2) was used to estimate the octanol-water partition coefficient (P_{octanol-water}) of the compound's neutral species and of each of the ionized species that exist at pH 7.4 and 8.0. This trained algorithm is based on a well-accepted substructure approach [20A], refined to handle ionic species. Default ionic strength conditions were specified for these calculations. Estimated values of pKa and P_{octanol-water} were verified against published experimental values whenever available. The Geometry calculator (v. 5.3.2) was used to estimate the following parameters of molecular geometry from structures rendered in three dimensions according to van der Waals atomic radii: 1) smallest two-dimensional molecular surface (in Å²) to be projected from the three-dimensional structure; 2) molecular length measured perpendicularly to this plane of projection (in Å); 3) molecular volume (in Å³). Compounds were rendered as the lowest energy conformer. Stereospecificity of compounds with chiral centers was not considered.

2.2 Assessment of Model Predictive Power

Proton shuttle activity predicted on the basis of a proposed mathematical implementation of the theoretical framework was compared against experimentally-measured uncoupling activity for a test set of 48 structurally-diverse naturally-occurring phenolic compounds spanning a wide range of activity. Uncoupling was measured in isolated rat liver mitochondria treated at 100 μM, as reported elsewhere [12A]. Predicted and measured activity, as well as calculated physicochemical predictors of activity are summarized in Supplemental Data Table 1; compounds are listed by class and are identified by traditional phytochemical nomenclature, Chemical Abstract Service (CAS) registry identifier, and string structure in simplified molecular input line entry specification (SMILES). Fit was assessed by Spearman rank order correlation analysis for non-parametric data.

2.3 In-Silico Screening

The proton shuttle activity of 252 other naturally-occurring phenolic compounds was assessed in-silico using the developed model.

2.4 Design of Synthetic Derivatives

Novel compounds optimized for high activity were designed from naturally-occurring templates identified as being conducive to proton shuttle activity. Predicted activity and calculated physicochemical predictors of activity of these compounds are summarized in Supplemental Data Table 2; compounds are listed by template and are identified by SMILES string structure.

3. Theoretical Framework

3.1 Thermodynamic Considerations of Uncoupling Mediated by Proton Shuttles

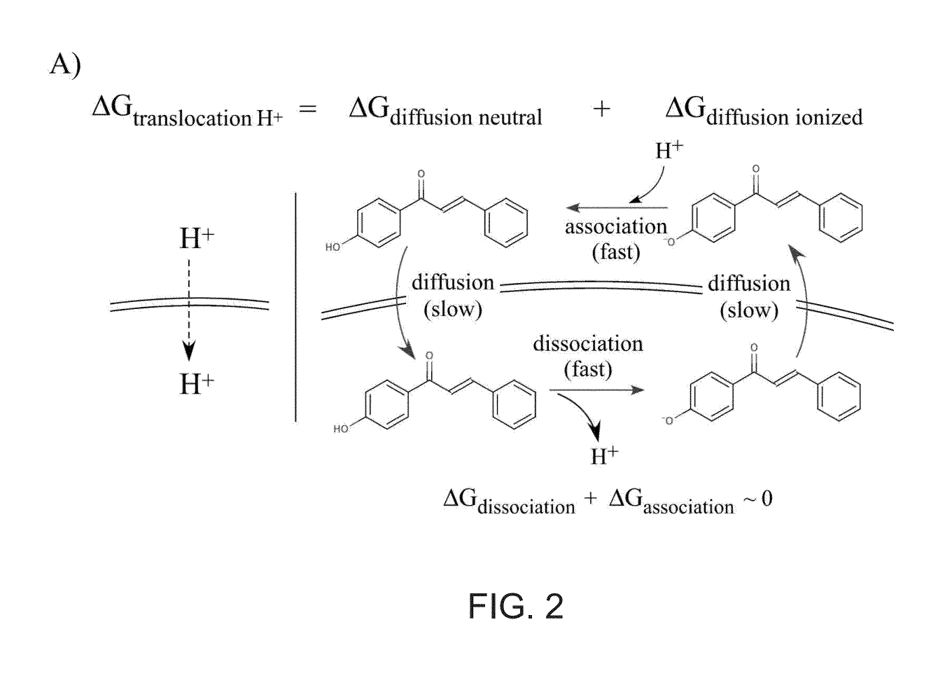

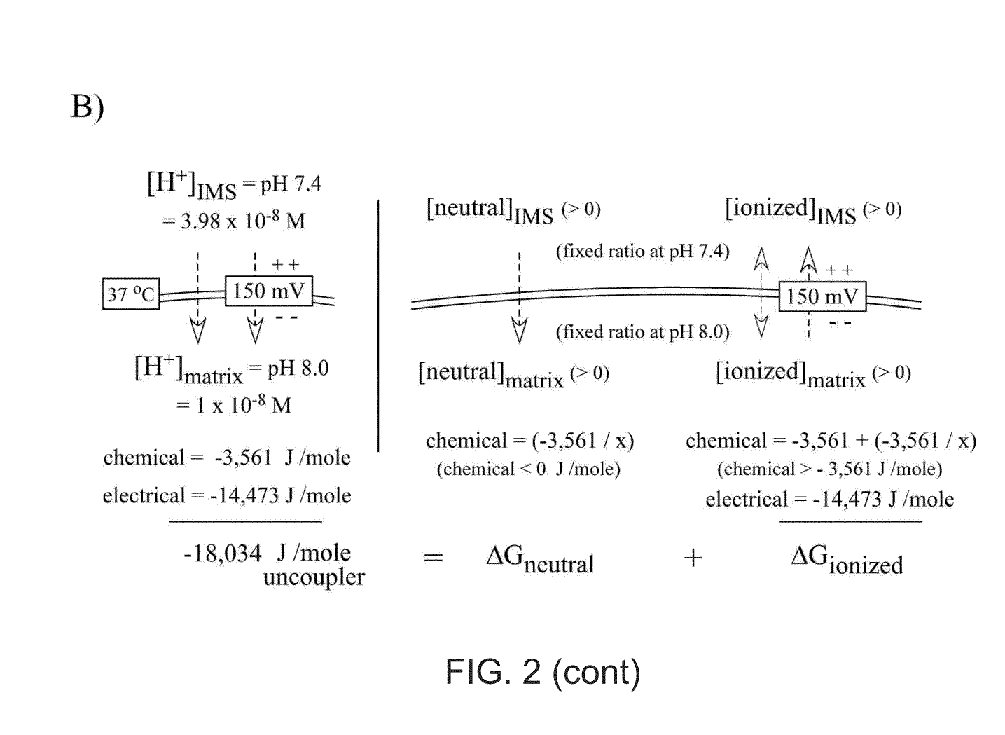

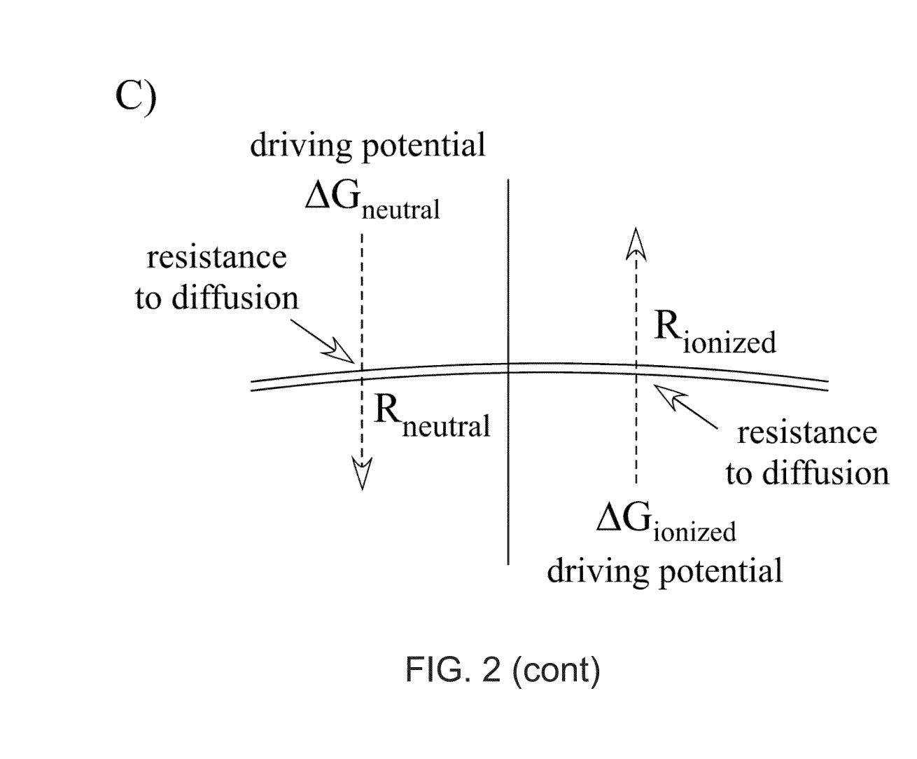

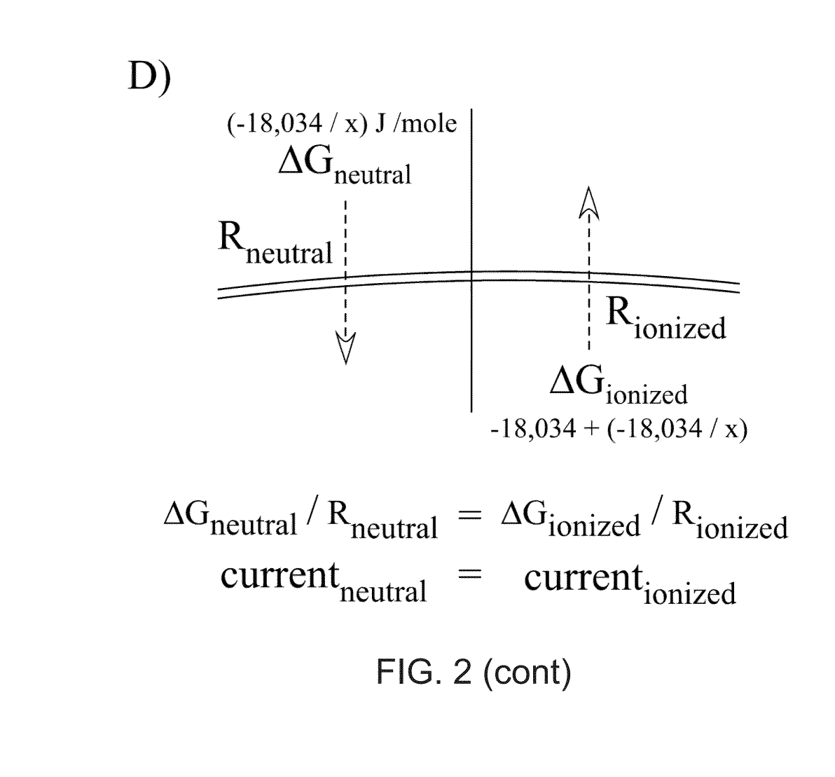

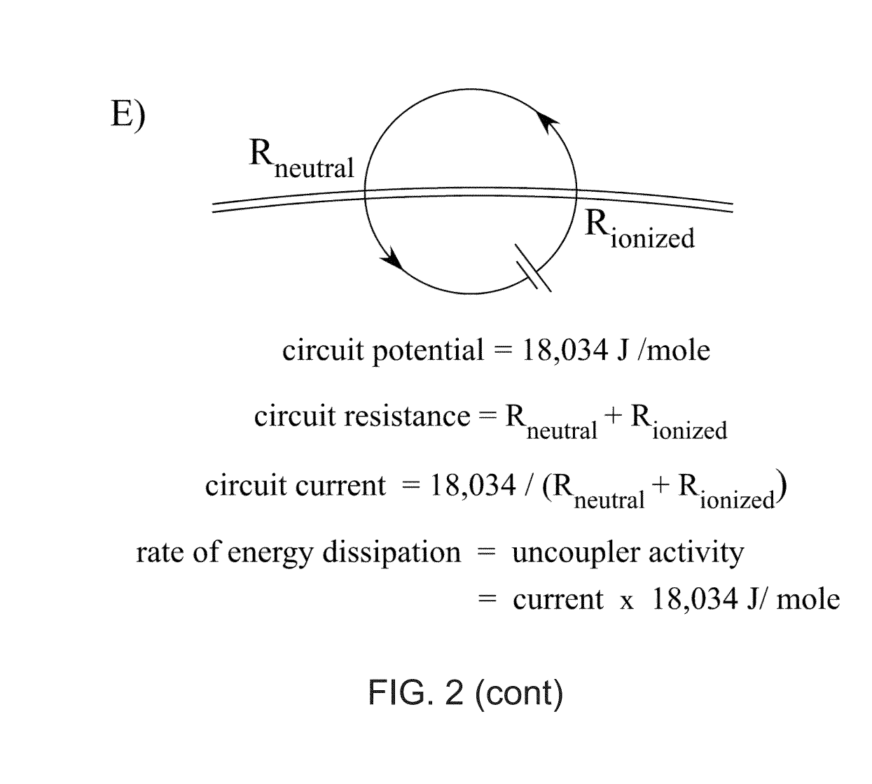

Perpetual disequilibrium and an ensuing cycle consisting of the diffusion of one molecule of the neutral species of an uncoupler from the mitochondrial inter-membrane space (IMS) to the matrix coupled to the diffusion of one molecule of the ionized species of the uncoupler from the matrix to the IMS, the end result of which is the translocation of a membrane-impermeable proton from the IMS to the matrix with each iteration (FIG. 1), lead to a premise of energetic coupling: each iteration of the cycle must be driven by the potential energy released in the translocation of the proton down its electrochemical gradient (FIG. 2A). Assuming the exchange of one molecule of the neutral species for one molecule of the ionized species to be coupled by mass action and to occur simultaneously, then the sum of (1) the energy dissipated in the driving of one molecule of the neutral species from the IMS to the matrix, (2) the energy released by the dissociation of one molecule of the neutral species in the matrix, (3) the energy dissipated in the driving of one molecule of the ionized species from the matrix to the IMS, and (4) the energy invested in the association of one molecule of the ionized species and a proton in the IMS must equal the energy released by the translocation of one proton from the IMS to the matrix. Energetically, the acid-dissociation and -association steps cancel out. Kinetically, these steps are much faster than the diffusional steps and therefore have a negligible effect on the overall rate at which the cycle can operate. The cycle can therefore be reduced to two diffusional steps, the sum of whose Gibbs free energy (A/G) equals that of the proton that they translocate.

The implication of this premise is that all (mono-protic) proton shuttles are driven by the same potential per mole, regardless of chemical species, physicochemical properties, or distribution of neutral and ionized species. Specifying parameters of: 37° C.; IMS pH 7.4; matrix pH 8.0; membrane potential 150 mV negative inside (a proton concentration gradient of 0.6 pH units and an electrical gradient of 150 mV combine for a proton motive force equivalent to 187 mV); and using the convention of attributing negative values to exergonic reactions, the ΔG for the translocation of protons across the inner membrane is calculated by the Nernst equation to be the sum of a −3,561 J./mole chemical component and of a −14,473 J./mole electrical component (FIG. 2B). As the electrical energy driving ionized uncoupler molecules from the matrix to the IMS is also −14,473 J./mole, the −3,561 J./mole balance is equal to the sum of the chemical energies driving the diffusion of neutral molecules and the diffusion of ionized molecules. Alternatively, any combination of neutral and ionized species concentrations constrained by the inviolable IMS and matrix patterns of speciation (dictated by compound pKa and the respective pH of these compartments) results in a −3,561 J./mole sum of chemical energies.

Although this implies that an infinite number of combinations of neutral and ionized concentrations are allowed, it is premised that a unique combination confers maximal efficiency upon the system, resulting in a maximal rate of cycling, and that this combination is determined by the ratio of the permeabilities of the neutral and of the ionized species. If it is also postulated that the system will tend to adopt conditions that confer maximal efficiency, then, at steady-state, this unique combination of concentrations will be favored over all others. The rationale behind this premise can best be explained by discussing permeability is terms of resistance to diffusion (taken to be the inverse of permeability) and applying electrical principles. Each diffusional step can be considered as having its intrinsic resistance (typically greater for the diffusion of the ionized species than for that of the neutral species) and its own driving potential (FIG. 2C). From this, flux or “current” (this term need not be reserved for the diffusion of ionized uncoupler molecules) through each step can be calculated by Ohm's law as potential divided by resistance. Assuming again the exchange of one neutral molecule for one ionized molecule to be coupled by mass action and to occur simultaneously, the system composed of two diffusional steps will be in its most energetically efficient state (i.e., will cycle at its maximal rate) if the average fluxes of both steps are equal (FIG. 2D). Conversely, if average fluxes are not equal, then the slower of the two processes will be rate-limiting to the overall cycle. Steady-state is therefore achieved by a balancing of concentrations within the constraints of patterns of speciation so as for the potential of each step to be proportional to the resistance of that step. This is therefore the premise of equal and maximal flux. From this, the system at steady-state can be considered to be a simple circuit (FIG. 2E) with an overall potential of 18,034 J./mole and with two resistances in series and therefore additive; the current through such a circuit is calculated as potential divided by the sum of the two resistances. Finally, the rate at which energy is dissipated by this circuit, the endpoint of an uncoupler's activity, is calculated as the product of current and potential. Accordingly, the permeabilities or resistances to diffusion of the neutral and ionized species of a proton shuttle are prime determinants of its activity.

3.2 Proposed Resistance-Lowering Effect of Asymmetric Molecular Distribution

According to the above, activity of a proton shuttle is determined by the ratio of the resistances to diffusion of its neutral and ionized species while acid-dissociation behavior (i.e., speciation at mitochondrial pH) merely constrains to fixed ratios the respective IMS and matrix concentrations of the two species. However, it is here proposed that acid-dissociation behavior is also a determinant of activity. More specifically, pKa and resistance to diffusion are proposed to be non-independent predictors of activity such that the resistance of the ionized species is effectively reduced with decreasing pKa, all other considerations being equal and within the limits of pKa (i.e., minimum pKa^{˜}4 for mono-protic acids).

This can be elaborated by considering the distribution of an uncoupler between four pools, the neutral_{IMS}, ionized_{IMS}, neutralₘₐₜᵣᵢₓ, and ionizedₘₐₜᵣᵢₓ pools, and by introducing notions of active fraction (or, in electrical terms, of charge carrier density) and of molecular excess. The ratio of distribution between these pools is determined by: 1) pKa and corresponding IMS and matrix patterns of speciation; 2) predicted steady-state concentrations (in turn dictated by the ratio of the resistances to diffusion of the neutral and ionized species and by patterns of speciation, as described in Section 3.1); and 3) compartment volumes, the volume of the matrix being 20 or more-fold greater than the volume of the IMS. For example, in the case of the compound 4′-hydroxychalcone from FIG. 1, given a pKa of approximately 7.9 and a ratio of the resistances to diffusion of the neutral and ionized species of approximately 1:22 (the estimation of resistance to diffusion is the object of the following section), then a cytosolic concentration of 100 arbitrary units of this compound is expected to result in the following steady-state concentrations (in the same arbitrary units of concentration): [neutral_{IMS}]=74.8; [ionized_{IMS}]=25.2; [neutralₘₐₜᵣᵢₓ]=55.5; [ionizedₘₐₜᵣᵢₓ]=74.3; under the following conditions: 37° C.; IMS pH 7.4; matrix pH 8.0; membrane potential 150 mV; cytosolic volume>>mitochondrial volume. (This corresponds to ΔG of −771 J./mole for the diffusion of the neutral species and of −17,263 J./mole for the diffusion of the ionized species). If the volume of the IMS is such that this compartment contains a total (neutral+ionized) of 100 molecules at the specified cytosolic concentration, and if the volume of the matrix is specified to be 20-fold that of the IMS, then these concentrations translate to the following approximate number of molecules in each pool at any given time: neutral_{IMS}=75; ionized_{IMS}=25; neutralₘₐₜᵣᵢₓ=1110; ionizedₘₐₜᵣᵢₓ=1486. Assuming yet again that the diffusion of one molecule of the neutral species from the IMS to the matrix is coupled by mass action to the diffusion of one molecule of the ionized species from the matrix to the IMS, it can be proposed that the smaller of the two counts of neutral_{IMS} and ionizedₘₐₜᵣᵢₓ molecules (75 in the present example) corresponds to the number of one-molecule exchanges simultaneously mediated by the proton shuttle at any given time at the specified cytosolic concentration; therefore, at any given time, only 75 neutral molecules and 75 ionized molecules in the present example are in movement and directly involved in the shuttling of protons at this specified concentration. In of itself, this active fraction, which varies from one proton shuttle to another at a given cytosolic concentration, has no impact on current or activity, but merely defines the rate of diffusion at a given current, within physical limits of terminal velocity; this is analogous to the linear relationship between charge carrier density and drift velocity in conductors of different materials within a same circuit. However, dividing the larger of the two counts of neutral_{IMS} and ionizedₘₐₜᵣᵢₓ molecules by this number of simultaneous one-molecule exchanges provides a measure of distribution asymmetry: a 20-fold excess in favor of the ionized species in the present example. From this, it is proposed that the resistance of the diffusional step benefiting from a molecular excess is effectively decreased in proportion to the magnitude of this excess.

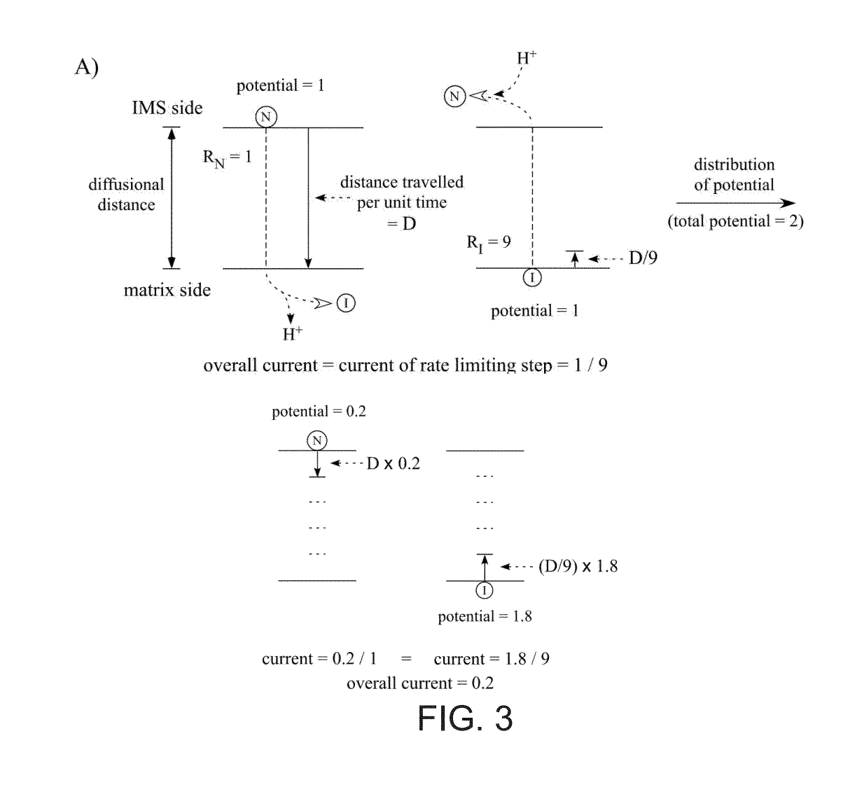

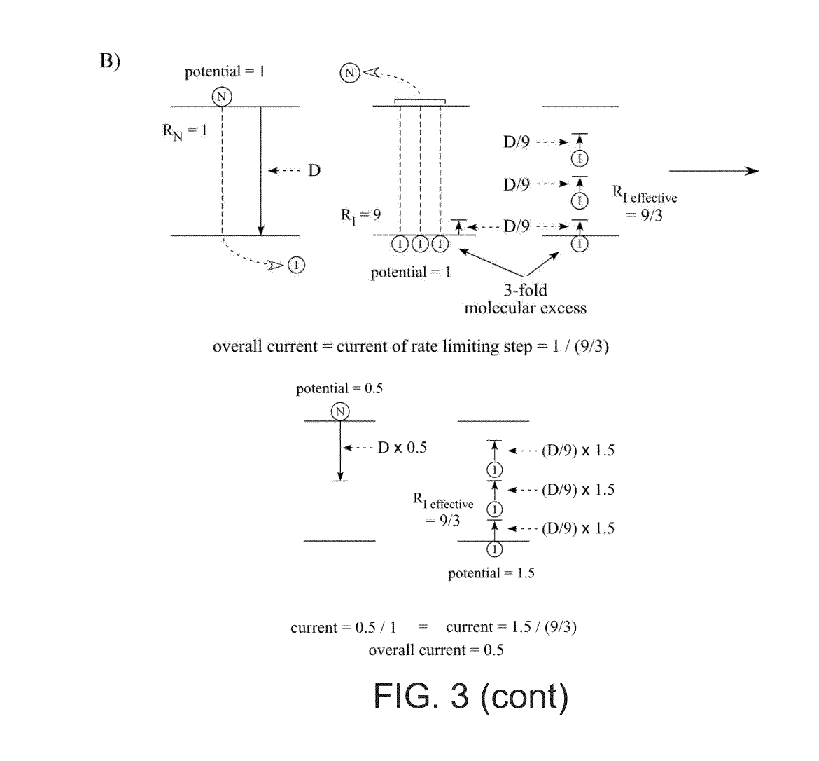

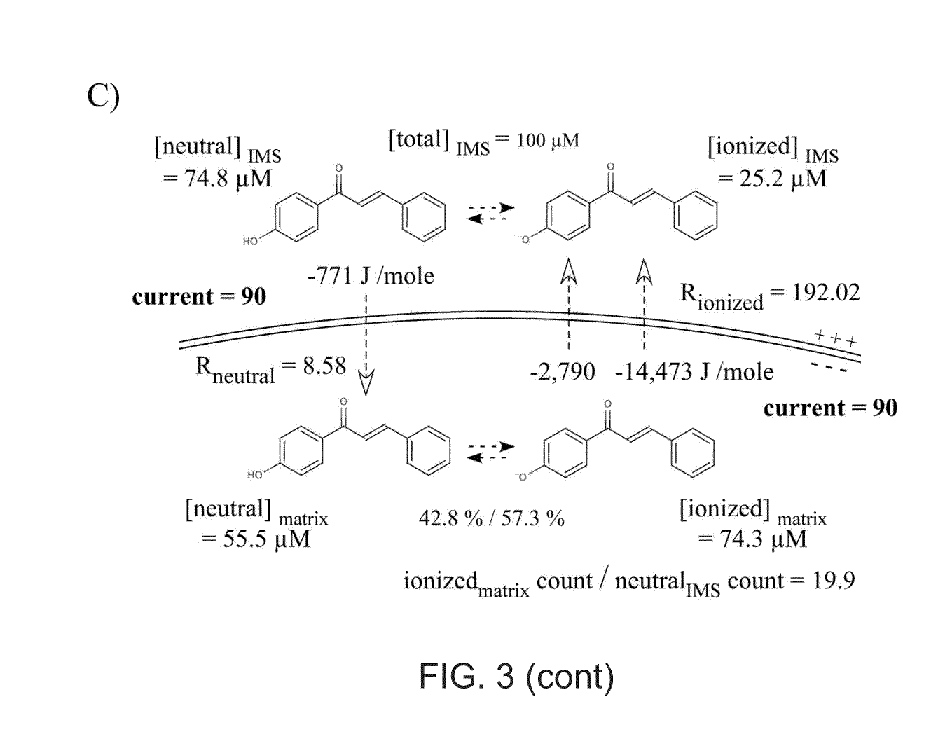

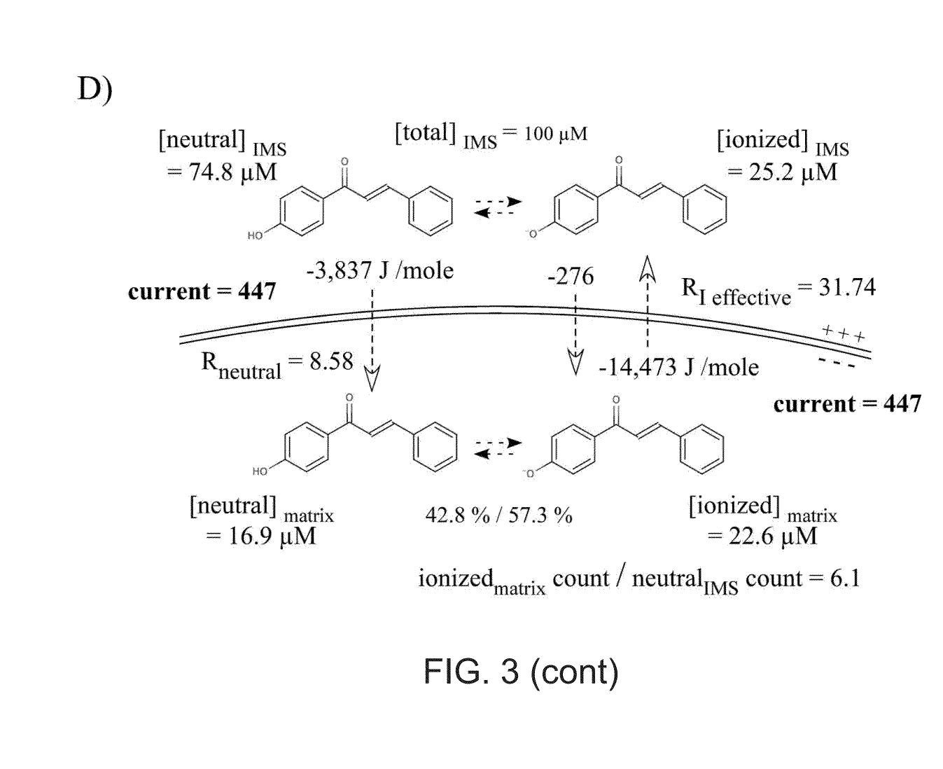

The rationale for this proposed resistance-lowering effect of asymmetrical distribution can best be presented using a simplified model system, as depicted in FIGS. 3A and 3B. In this system, potential is not linked to concentration, nor is there a transmembrane pH gradient and resulting differential speciation. As always, a cycle consists of a coupled and simultaneous exchange of a molecule of the neutral species from the IMS side of the system for a molecule of the ionized species from the matrix side, and stoichiometry is maintained by the concurrent dissociation of a neutral molecule on the matrix side and association of an ionized molecule and a proton on the IMS side (not depicted). It is assumed that a diffusional distance equal to at least some portion of the width of the membrane separates the sites of association and dissociation, that diffusion is a process distinct from the transfer of molecules between the ionizedₘₐₜᵣᵢₓ and ionized_{IMS} pools or between the neutral_{IMS} and neutralₘₐₜᵣᵢₓ pools, and that the boundaries of the neutral_{IMS} and ionizedₘₐₜᵣᵢₓ pools extend into the diffusional distance. Under the scenario of FIG. 3A, there is a single molecule in each of the neutral_{IMS} and ionizedₘₐₜᵣᵢₓ pools, whereas under the scenario of FIG. 3B, there is a single molecule in the neutral_{IMS} pool but three molecules in the ionizedₘₐₜᵣᵢₓ pool; the number of molecules in the other pools need not be specified. As treated in Section 3.1, the two excess molecules in the second scenario do not directly participate in the coupled exchange during a given cycle iteration. Nevertheless, it can be proposed that simultaneously with the diffusion of the participating ionized molecule from the ionizedₘₐₜᵣᵢₓ pool, the excess ionized molecules are driven to diffuse at least some intermediate distance towards the ionized_{IMS} pool before their diffusion is opposed by mass action. Accordingly, upon the subsequent iteration of the cycle, the transfer from the ionizedₘₐₜᵣᵢₓ pool to the ionized_{IMS} pool can be expected to draw upon one of these excess molecules, now physically closer to the ionized_{IMS} pool, rather than upon the molecule newly-formed by the dissociation step and which must bridge the full distance to the ionized_{IMS} pool; by contrast, molecules from the neutral_{IMS} pool must always bridge the full distance. As a result, the time needed to complete the ionized species' diffusional step is reduced relative to that of the initial cycle iteration. In subsequent iterations, ionized molecules closest to the IMS are again preferentially drawn upon. If, as a simplifying assumption, the partial diffusion distance is taken to be equal to the entire distance between the ionizedₘₐₜᵣᵢₓ and ionized_{IMS} pools, then, within a few iterations, this phenomenon can be expected to result in the equidistant staggering of excess ionized molecules across the entire diffusional distance, at which point, the effective resistance of this diffusional step is reduced in proportion to the molecular excess.

In a real system where potential and distribution are indissociable, it must be considered that whenever the resistance of a diffusional step is effectively reduced on the basis of asymmetric distribution, then calculations of steady-state distribution must be revised, in turn reducing the computed magnitude of molecular excess and of the resistance-lowering effect. Stated differently, as the ratio of the resistances to diffusion of the neutral and ionized species becomes more balanced due to a resistance-lowering effect of distribution, the forces driving the two diffusional steps also become more balanced so as to maintain a maximally efficient system, the consequence of which is a reduction in the asymmetry of molecular distribution and of the magnitude of the resistance-lowering effect. (This apparent circularity can be resolved using an optimization routine: in the proposed mathematical implementation (available at doi: 10.1016/j.jtbi.2012.02.032), steady-state concentrations and molecular distribution are initially calculated from a resistance-lowering effect magnitude arbitrarily set to 1; this value is then systematically increased and calculations repeated until it is numerically equal to the magnitude of molecular excess calculated from the corresponding distribution). Returning to the example of 4′-hydroxychalcone, the actual resistance-lowering effect is therefore calculated to be of approximately 6-fold rather than 20-fold as estimated earlier. Calculations of steady-state conditions for this compound, performed with and without taking into account the proposed resistance-lowering effect, are summarized in FIGS. 3C and 3D, respectively.

Note that while the notion of resistance-lowering effect has been developed above in such a way that molecular excess always favors the species with the highest native resistance, the notion applies as well to the reverse situation such as can be expected of a compound with pKa above mitochondrial pH. For example, the compound 3-hydroxychalcone (pKa^{˜}9.4; ratio of the resistances to diffusion of the neutral and ionized species^{˜}1:28) can be expected to have the following steady-state concentrations under the same conditions specified above: [neutral_{IMS}]=99.0 a.u.; [ionized_{IMS}]=1.0 a.u.; [neutralₘₐₜᵣᵢₓ]=84.2 a.u.; [ionizedₘₐₜᵣᵢₓ]=3.3 a.u.; its neutral species thereby benefiting from a 1.5-fold resistance-lowering effect of distribution. However, decreasing the lower of the two resistances has significantly less impact on current, and hence on activity, than decreasing the higher of the resistances by the same factor. In such cases, the effect can therefore be considered negligible and ignored. For rigor, however, the effect is calculated symmetrically throughout this work.

3.3 Estimating Membrane Permeability/Resistance to Diffusion

Activity of a proton shuttle is proposed to be determined by the membrane permeabilities of its neutral and of its ionized species, as well as by its acid-dissociation behavior. In order to predict activity for the purposes of in-silico screening of known compounds or of guiding the design of novel ones, acid-dissociation and permeability behavior must be known or must be reasonably-well estimated. While there exist algorithms for the reliable estimation of pKa from compound structure, from which speciation at any given pH can then be calculated, there are no comparable algorithms for directly estimating membrane permeability. Instead, permeability must be predicted from physicochemical parameters such as lipid partitioning behavior. The present section proposes an approach to the estimation of the membrane permeability of both the neutral and ionized species of a proton shuttle that is based on structure-derived calculations of lipid partitioning behavior and of compound geometry. The contributions to permeability of these predictors are assessed separately and in relative terms, then multiplied together to give relative permeability.

3.3.1 Contribution of Partitioning Behavior to Resistance to Diffusion

The most important physicochemical predictor of a compound's membrane permeability is undoubtedly lipophilicity [21A], or more specifically, deviation from the ideal degree of lipophilicity for transmembrane diffusion. In keeping with the notion of resistance, a deviation from optimal lipophilicity can be considered to increase the viscosity of the interaction between the diffusing compound and the molecules of the medium in which diffusion is occurring, thereby increasing the magnitude of the frictional forces that retard a compound's diffusion and limiting its rate of diffusion under a given driving potential. Both positive and negative deviations will increase the degree of mismatch in hydrophilicity/lipophilicity behavior or polar nature, and hence reduce permeability. The ideal degree of lipophilicity for transmembrane diffusion represents a compromise between an optimum for diffusion through the membrane's hydrophilic boundary layers and outer surfaces and an optimum for diffusion through the membrane's hydrophobic core. The ideal degree of lipophilicity for transmembrane diffusion is therefore necessarily higher than that for transcellular diffusion (determined, for example, from the study of rates of absorption of small molecule drugs) which represents a compromise between the requirements for efficient transmembrane diffusion and those for aqueous diffusivity.

Lipophilicity of a compound is traditionally measured as the compound's partitioning ratio between the model lipid octanol and water (P_{octanol-water}), expressed on a log scale such that log P_{octanol-water} of 0 denotes equal partitioning and log P_{octanol-water} of +3.0 indicates a 1000-fold greater affinity for octanol than for water. This measure largely captures notions of polarity and therefore needs not be augmented with a separate measure of polar surface area. While P_{octanol-water} does not translate directly to partitioning between a biological membrane and water or intracellular fluid [21A], this measure of lipophilicity nevertheless offers good resolution and can be reliably calculated for both neutral and ionized species of a given compound with well-established segment-based algorithms. Expressed in these terms, optimal lipophilicity for absorption of small molecule drugs is reported to be on the order of log P_{octanol-water} of 2.0 [21A]. Based on empirical observations of proton shuttle physicochemical data, the optimal P_{octanol-water} value for transmembrane diffusion can be expected to be at least an order of magnitude greater than this value. The relationship between permeability and octanol-water partitioning is generally regarded as linear over several orders of magnitude, with a slope of approximately unity [21A]. Accordingly, permeability can be modeled to decrease in direct proportion with negative deviations from a specified log P_{octanol-water} optimum. The relationship is less clear at supraoptimal values of lipophilicity [21A], but for simplicity, permeability can be modeled to decrease with positive deviations from the specified optimum at the same rate as for negative deviations.

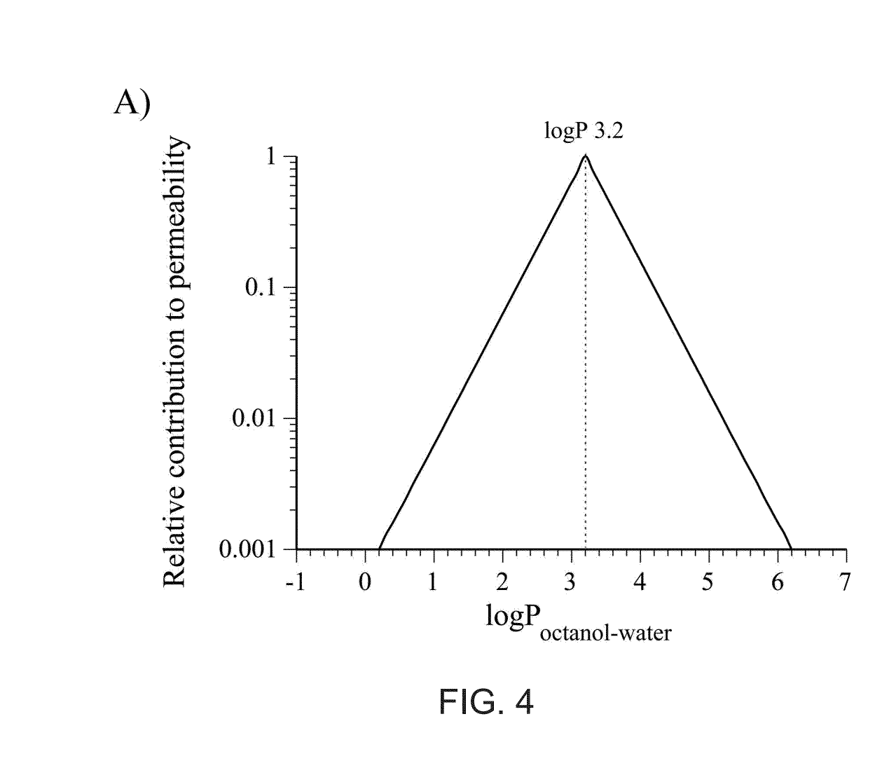

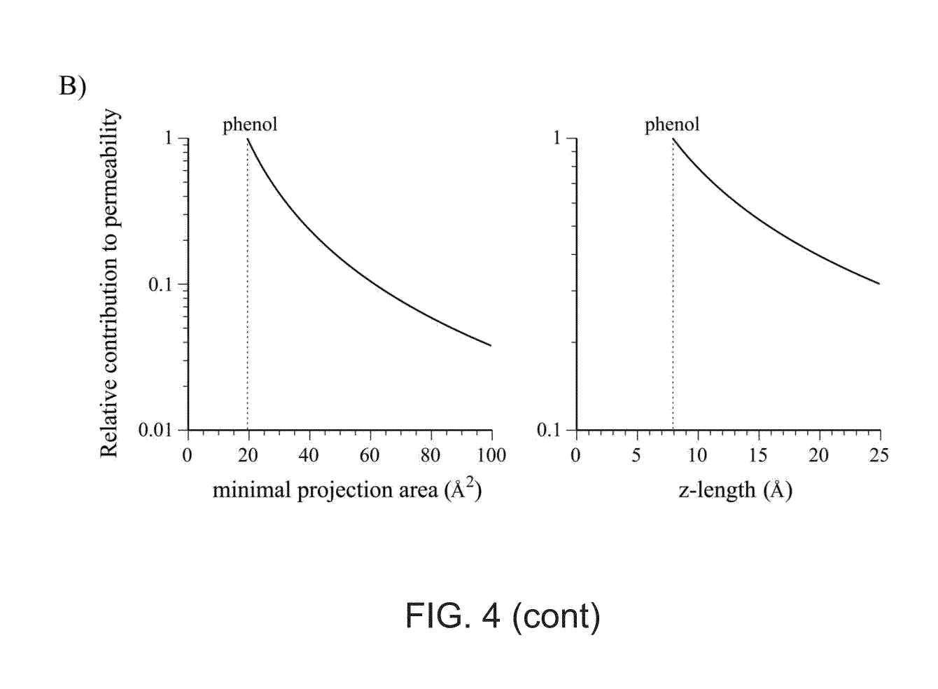

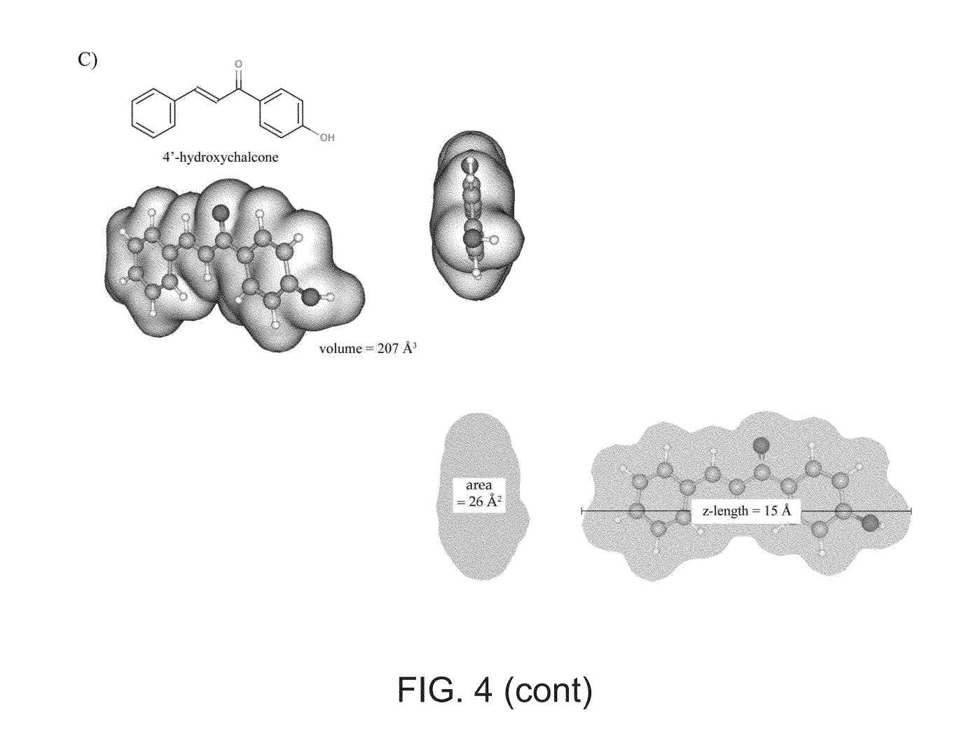

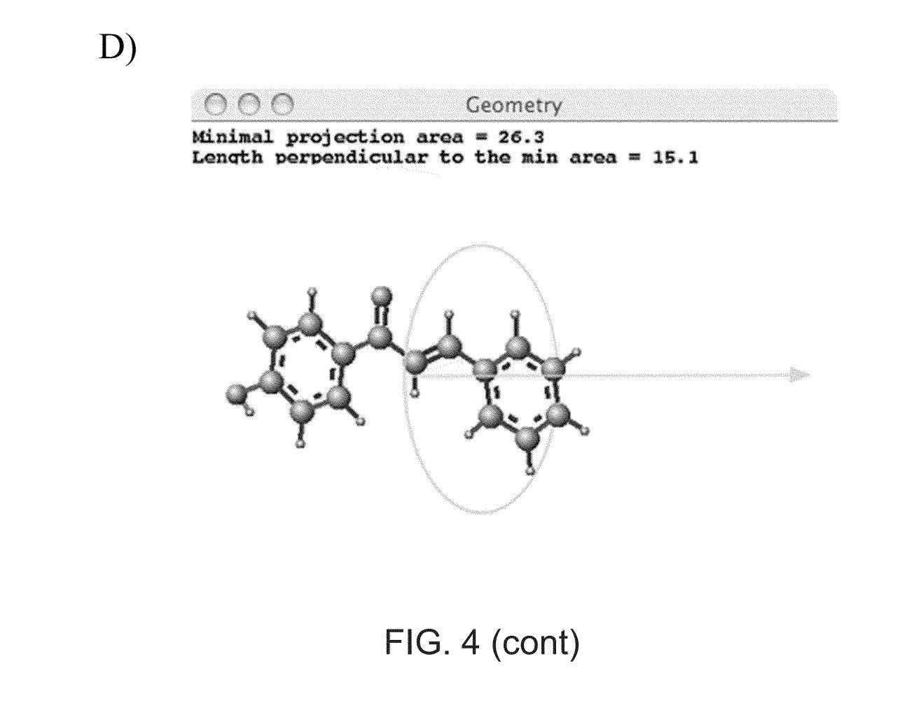

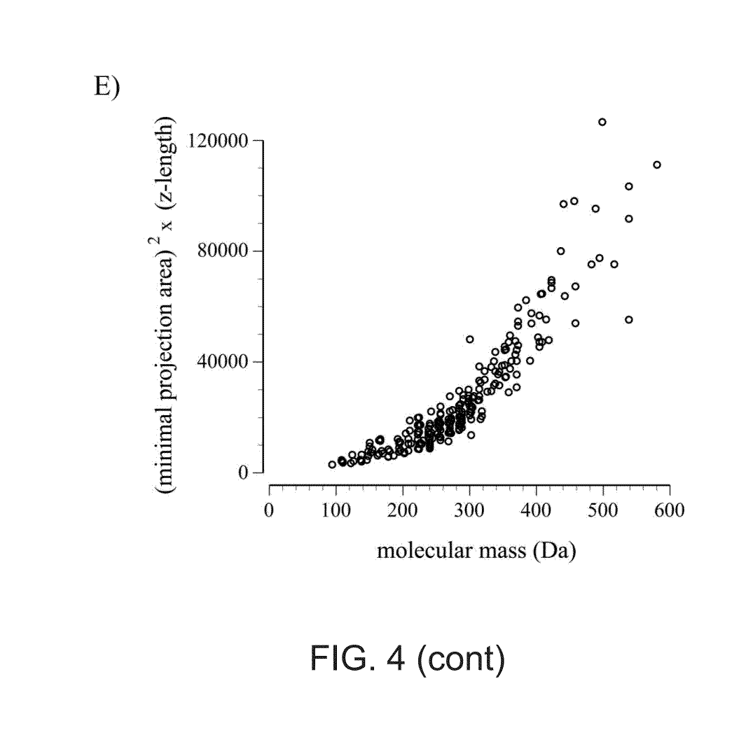

These notions are implemented as follows. The optimal log P_{octanol-water} value for transmembrane diffusion is set to 3.2 on the basis of the best fit between predicted proton shuttle activity and measured uncoupling activity for 48 compounds (Section 4.1, below). At this optimum, the relative contribution of lipophilicity to permeability is set to 1. For compounds with log P_{octanol-water} values of less than 3.2, the contribution to permeability is taken to be equal to the log 10 transformation of 3.2 minus the compound's log P_{octanol-water} value. Conversely, at values above 3.2, the contribution to permeability is taken to be equal to the log 10 transformation of the compound's log P_{octanol-water} value minus 3.2. This bilinear function is illustrated in FIG. 4A.

Evaluation of the contribution of lipophilicity to permeability is performed independently for the neutral and ionized species of a proton shuttle. Ionized species invariably exhibit less affinity for octanol than their corresponding neutral species. The magnitude of this decrease in P_{octanol-water} ranges from less than 100-fold in compounds capable of extensive charge delocalization over a ring system, to over 3000-fold in compounds with a localized charge, such as carboxylic acids; a 150-fold decrease in P_{octanol-water} upon ionization is typical of the phenolic compounds considered in the present work. The bilinear function described is applied to neutral and ionized species in the same manner. It follows from this that the ionized species of an excessively lipophilic compound can be predicted to be more membrane permeable than its corresponding neutral species. Implications of this are addressed in Section 3.4, below. It should be noted that shielding of an ionization site with bulky substituents, suggested by others to contribute to activity [8A], has no effect on the magnitude of decrease in lipophilicity upon ionization.

Finally, it should be noted that many naturally-occurring compounds, including several of the compounds considered in Sections 4.1 and 4.2, below, occur as glycosides. Given that glycosylation reduces P_{octanol-water} by two or more orders of magnitude while also simultaneously contributing to molecular bulk (addressed next), glycosides can generally be considered devoid of proton shuttle activity. Therefore, only the aglycone form of such compounds is considered in the present work, and it is recommended that glycosides always be evaluated as the corresponding aglycone. Along similar lines, naturally-occurring compounds that occur as large polymers (e.g., tannins) can also be expected to be devoid of activity in polymeric form on the basis of excessive lipophilicity and molecular bulk. These should therefore be evaluated as their respective building blocks or as small oligomers with appropriate lipophilicity and geometry (see Section 3.6.3, below).

3.3.2 Contribution of Molecular Size and Shape to Resistance to Diffusion

In addition to lipophilicity, a compound's permeability behavior, and hence its proton shuttle activity, is determined by geometric considerations. Indeed, it is well known that the magnitude of the frictional forces that retard a compound's diffusion increase with molecular size, and that, other physicochemical properties being equal, a smaller compound diffuses more readily through a membrane than a larger one. The impact of molecular size on membrane permeability can be estimated by borrowing from notions of aqueous diffusivity (i.e., Stokes-Einstein relationship): for model compounds of spherical shape, the magnitude of frictional forces varies with compound volume and can be estimated from molecular mass alone, with no other geometric considerations, since the volume of a spherical compound can be shown to depend only on molecular mass; when dealing with asymmetric compounds, molecular mass is corrected by an estimate of deviation from sphericity, under the assumption that for any given mass, a non-spherical compound has a larger volume than a spherical compound and must therefore be subjected to frictional forces of greater magnitude. These notions, however, assume the size of the diffusing compound to be larger than that of the molecules of the medium in which diffusion is occurring, and therefore Brownian motion to be unconstrained; accordingly all possible orientations of an asymmetric compound are equally favored and permeability behavior can be taken to be the average of the effects of each orientation.

As such, these notions do not well predict the frictional forces acting on an asymmetric compound as it is driven through the decidedly anisotropic architecture of a biological membrane, especially if that compound's mass is on the same order as that of the membrane lipid constituents that it must intercalate. Rather, it can be expected that such a compound will be constrained to adopt an orientation whereby its shortest axis is roughly perpendicular to the long axis of membrane lipids and to the direction of travel, since all other orientations will require that a larger hole be opened in the membrane. Stated differently, the orientation that minimizes the surface area perpendicular to the direction of travel (i.e., “frontal area”) can be expected to be favored as it will minimize the magnitude of frictional forces. From this, it may be reasonable to adopt the simplifying assumption of anisotropic diffusion when estimating the contribution of compound size and shape to permeability, and therefore to make this estimate on the basis of the magnitude of frictional forces incurred in the preferred orientation. Accordingly with notions of viscous drag, magnitude of frictional forces can then be taken to be proportional to the square of the compound's “minimized frontal area” and proportional to its length measured perpendicularly to the plane of the minimized frontal area, or the “z-length.” It follows from this that higher rates of diffusion can be achieved by linear and planar compounds than by globular compounds of equal mass, including perfectly spherical compounds. More broadly, it follows that permeability/resistance cannot be determined by size alone, but instead that the interaction of size and shape must be considered. This is consistent with empirical observations that planar compounds are more conducive to proton shuttle activity [22A, 23A]. However, it may not be in complete agreement with results from studies of rates of absorption of small molecule drugs, since, as mentioned above, transcellular diffusion is an aggregate measure of membrane permeability and aqueous diffusivity.

The above notions are implemented as follows. Membrane permeability is taken to decrease with the square of the minimized frontal area, as well as linearly with z-length. The contribution of both of these geometric parameters to permeability is therefore modeled as much smaller than that of lipophilicity. Measures of minimized frontal area and of z-length are normalized to the dimensions of the compound phenol, the smallest structure common to all compounds of interest in the present work, whose respective contributions to permeability are arbitrarily set to 1. These functions are illustrated in FIG. 4B. Frontal area is taken to correspond to the surface area projected from the three-dimensional rendering of a compound in its lowest energy conformation and according to van der Waals atomic radii. The minimized frontal area is therefore taken to be the smallest surface area that can be projected as the rendering is rotated about its three axes, known as the minimal projection area. For phenol, this value is calculated to be 19.4 Å². Z-length is then assessed from any van der Waals surface perpendicular to the plane of the minimal projection area. For phenol, this value is calculated to be 7.9 Å. This is illustrated for 4′-hydroxychalcone in FIGS. 4C and 4D. These measurements are performed on the neutral species of the compound and are assumed to be valid for all ionized species as well; geometric considerations therefore do not factor into determinations of molecular distribution (Section 3.1).

Over the mass range of small molecule drugs, the product of the square of minimal projection area and of z-length better resolves asymmetric compounds than mass indices or other geometric indices such as molecular volume. To this point, the 283 non-ionic compounds of Sections 4.1 and 4.2, below, which range from 94 to 581 Da and from 91 to 486 Å³, span a 42.5-fold range of this index. Furthermore, this index captures the wide range of molecular shape possible at any given molecular mass; plotting the product of the square of minimal projection area and of z-length against molecular mass for these same compounds (FIG. 4E) shows a 2- to 3-fold spread in index values, the absolute importance of which grows with increasing molecular mass. Finally, the relationship that emerges from such a plot suggests that geometric considerations rapidly become prohibitive to diffusion above 300 Da, with a proposed exclusion limit on the order of 600 Da.

This index might be further improved by integrating the notion of dipole moment. Indeed, the orientation of any compound with a non-zero dipole moment (including ionized species) can be expected to be influenced by the electric field resulting from the transmembrane electrical potential. If the orientation dictated by the electrical field does not correspond to the orientation that minimizes frontal area, then the preferred orientation will be a compromise between these two. No attempt was made to account for this effect.

3.4 Implications of the Interaction of Acid-Dissociation Behavior and Resistance to Diffusion

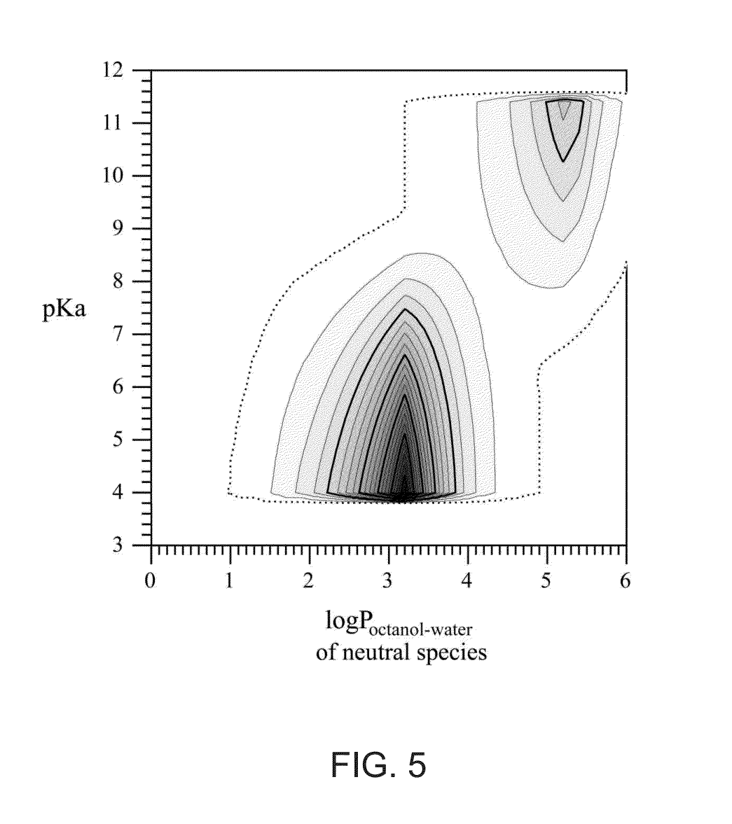

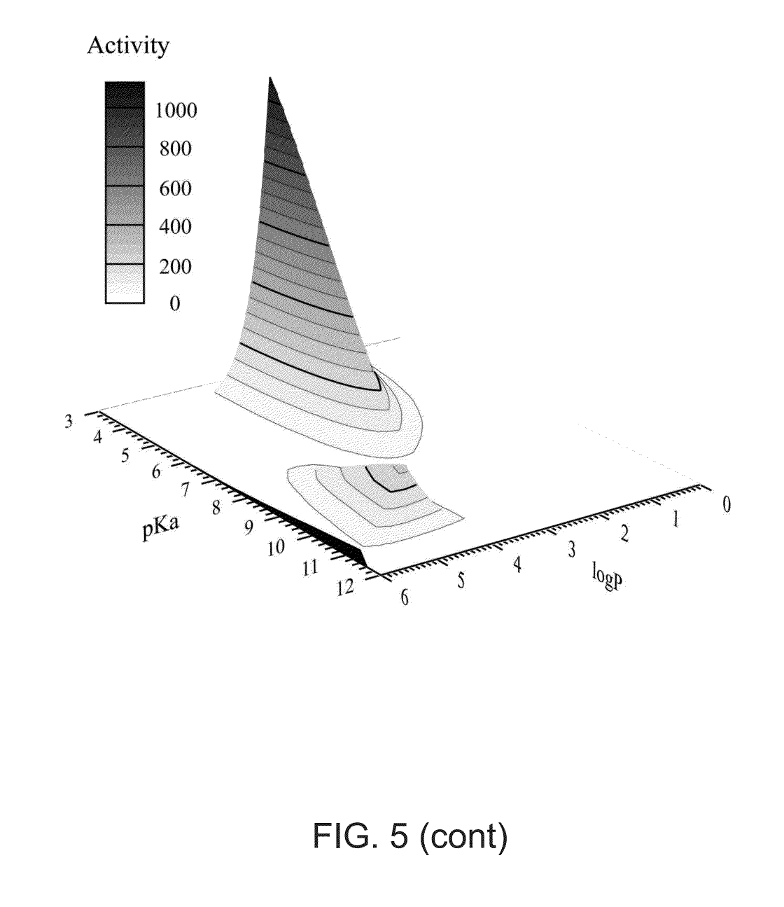

In Sections 3.1 and 3.2, it is proposed that proton shuttle activity is determined by acid-dissociation behavior and membrane permeability behavior, and that these parameters are independent as well as non-independent predictors of activity; they are proposed to be related through the premise of equal and maximal flux, whereby molecular distribution depends on patterns of speciation and on the ratio of the resistances to diffusion of the neutral and ionized species, and through a resistance-lowering effect of asymmetric distribution, a phenomenon resulting in an effective reduction of the resistance to diffusion of either the neutral or the ionized species under certain conditions of molecular distribution. This multi-level interaction and its implications may be best appreciated from of a surface plot of the activity predicted to result from all combinations of pKa and neutral species log P_{octanol-water} values for mono-protic compounds, as presented in FIG. 5.

Such a plot is made possible by controlling both the decrease in lipophilicity incurred upon ionization and the contributions to permeability of minimized frontal area and of z-length. In FIG. 5, the decrease in lipophilicity was fixed at 2.1 units of log P_{octanol-water}, a magnitude representative of phenolic compounds; the corresponding ionized species log P_{octanol-water} value at any plotted neutral species log P_{octanol-water} value is therefore taken to be equal to the neutral species value minus this constant. Specifying a fixed value for the geometric parameters is not appropriate, however, because molecular bulk and lipophilicity become tightly related at moderate to high lipophilicity. Rather, the contributions to permeability of minimized frontal area and of z-length were set to vary with the plotted neutral species log P_{octanol-water} value so as to always correspond to the minimum achievable at a given degree of lipophilicity by compounds composed exclusively of C, H, and O; the function used for this was derived from the relationship between log P_{octanol-water} value and the product of the square of minimized frontal area and of z-length for the 283 non-ionic compounds considered in Sections 4.1 and 4.2, below.

From such a plot, a principal physicochemical space of proton shuttle activity, or “activity space”, can be defined such that activity is maximized as the neutral species log P_{octanol-water} value approaches the optimal value for diffusion (specified to be 3.2), and as pKa approaches its minimum (specified to be 4.0). Low pKa maximizes the resistance-lowering effect of asymmetric distribution acting on the ionized species, thereby minimizing overall resistance to diffusion (i.e., sum of neutral species resistance and ionized species resistance; Section 3.1); as pKa decreases, the asymmetry of distribution increases in favor of the ionized species, and, consequently, the contribution of the ionized species to overall resistance is decreased. Over the pKa range of 4 to 8, the resistance-lowering effect is of such magnitude that the neutral species rather than the ionized species becomes the predominant contributor to overall resistance for any compound that exhibits greater permeability in neutral than in ionized form. Overall resistance is therefore minimized by optimizing the permeability of the neutral species at the expense of that of the ionized species. As such, the ideal neutral species log P_{octanol-water} value coincides with the optimum value for diffusion. Above pKa 8, the resistance-lowering effect tapers off, the contribution of the ionized species to overall resistance increases, and maximal activity is achieved by trading off permeability of the neutral species for increased permeability of the ionized species. Therefore, above pKa 8.0, the ideal neutral species log P_{octanol-water} value becomes greater than the optimum for diffusion. By extension, in the complete absence of a resistance-lowering effect, both species would contribute equally to overall resistance, and overall resistance would be minimized when both species exhibit equal permeability (i.e., log P_{octanol-water} value equally removed from the optimum; neutral log P_{octanol-water} Of 4.25 and ionized log P_{octanol-water} of 2.15, in the case of a decrease of 2.1 units upon ionization and an optimum of 3.2). Minor alterations in model parameters or in the shape of the function relating lipophilicity to permeability may affect the location or the magnitude of the activity peak, but are unlikely to significantly alter the general relationship. Along these lines, a partial collapse of the mitochondrial transmembrane pH gradient and reduction of the potential driving the proton shuttle cycle would reduce peak activity and rate of climb of the relationship without otherwise affecting the general relationship.

The activity space described above applies to compounds that are more membrane-permeable in neutral form than in ionized form. Symmetrically, a second activity space is defined for highly lipophilic compounds that are more permeable in ionized than in neutral form (as per the bilinear function defined in Section 3.3.1). This space is defined such that activity is maximized as the neutral species log P_{octanol-water} approaches the value corresponding to the optimum for diffusion plus the magnitude of the decrease incurred upon ionization (specified to be 3.2+2.1=5.3), and as pKa approaches its maximum (specified to be 11.4). At high pKa, distribution is such that the proposed resistance-lowering effect applies to the neutral species, and, therefore, overall resistance is minimized as the ionized rather than the neutral species log P_{octanol-water} value coincides with the optimum value for diffusion. The location of this peak is, of course, dependent on the specified decrease in lipophilicity incurred upon ionization. The reference protein-assisted uncoupler 2,6-bis(1,1-dimethylethyl)-4-methylphenol (BHT; pKa>11; estimated neutral/ionized species log P_{octanol-water} 5.27/2.89) can be proposed as an example of a compound whose proton shuttle activity is defined by this second activity space.

The peak of the second activity space can be expected to be considerably lower than that of the primary space. This is due to two factors. First, because the matrix volume is greater than the IMS volume, distribution is biased in favor of the matrix side, and the magnitude of the resistance-lowering effect is therefore greater when this effect applies to the ionized species (i.e., in the case of compounds with low pKa) than when it applies to the neutral species (i.e., in the case of compounds with high pKa). Second, molecular bulk increases significantly with increasing lipophilicity above a log P_{octanol-water} value of 4 for compounds composed exclusively of C, H, and O, and this can be expected to negatively impact permeability. This phenomenon is also responsible for the asymmetry around the log P_{octanol-water} axis in the primary space. In light of this activity handicap, and, more importantly, in light of issues associated with excessive lipophilicity (e.g., increased risk of toxicity due to bioaccumulation, non-specific binding, and more difficult metabolism; formulation issues), this second activity space is of limited interest from the perspective of uncoupler design, either for therapeutic or industrial purposes.

3.5 Special Considerations for Multi-Protic Proton Shuttles

A comprehensive theoretical framework of proton shuttle uncoupling must successfully reconcile not only compounds with a single ionizable site (i.e., mono-protic), but also compounds that exist as two or more ionized species at mitochondrial pH (termed multi-protic hereafter). Indeed, more often than not, xenobiotics with proton shuttle activity are multi-protic compounds; this is reflected by the compounds considered in Sections 4.1 and 4.2, below.

Like their mono-protic counterparts, multi-protic compounds must exist in both neutral and ionized forms in both the IMS and matrix as a condition for proton shuttle activity. It should be noted, however, that the pKa range compatible with the above condition tends to be considerably more restrained for multi-ionized species than for mono-ionized species, and the exact range of compatibility will vary according to intrinsic acid-dissociation properties.

The notion of multi-protic proton shuttles raises several mechanistic questions. For example, when a proton shuttle exists as several different ionized species and their corresponding neutral species, do more than one of the ionized species contribute to activity or is activity mediated only by the most “efficient” species? If the answer is the former, what is the contribution of each ionized species and how are molecules of the neutral species apportioned between them when their quantity is inferior to the combined quantity of molecules of the ionized species? If it is the latter, what qualifies a species as most efficient, and do the other ionized but non-participating species influence its activity? Finally, in considering the potential contribution of di-ionized or even other multi-ionized species, can the mechanistic advantage of translocating more than a single proton per cycle iteration more than offset the greatly reduced lipophilicity of such species relative to their mono-ionized counterparts? These questions are addressed here.

3.5.1 Thermodynamic Considerations

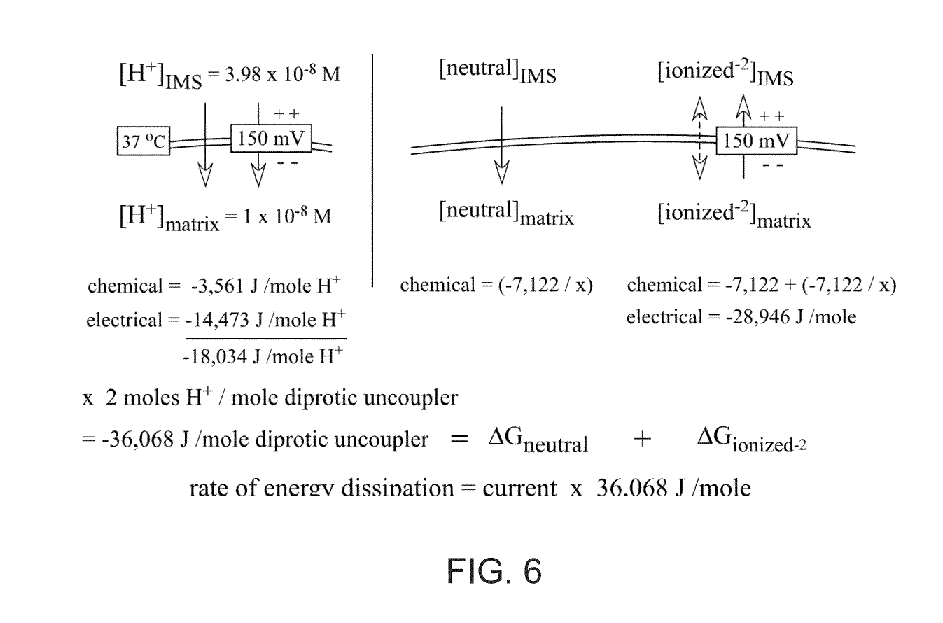

If it is assumed that protons may be shuttled across the mitochondrial inner membrane by a circuit composed of any combination of a multi-protic compound's neutral species and one of its ionized species, then it is necessary to adapt some of the thermodynamic notions of Section 3.1 to account for the possibility of the translocation of more than a single proton per cycle iteration. Thermodynamically, a cycle involving the neutral species and one of the mono-ionized species of a multi-protic proton shuttle can be considered in the same way as the cycle involving the neutral and the ionized species of a mono-protic compound (Section 3.1): such a cycle reduces to a circuit driven by a diffusion potential per mole uncoupler equal to the ΔG for the translocation of a mole of protons from the IMS to the matrix (−18,034 J./mole under the conditions specified in Section 3.1). However, a cycle involving the neutral species and a di-ionized species rather than one of the mono-ionized species reduces to a circuit driven by a diffusion potential per mole equal to twice the ΔG for the translocation of a mole of protons (−36,068 J/mole uncoupler), since two protons are translocated per cycle iteration (FIG. 6). Such a cycle obviously involves additional association and dissociation steps. However, as with the original two steps, these can be considered to energetically cancel out and to be faster than the diffusional steps. The premise of equal and maximal flux still applies, and this larger potential is distributed to the neutral and ionized species, as before, according to the ratio of their resistance to diffusion. Note, however, that the electrical contribution to the diffusion of the ionized species is also doubled (−28,946 J./mole), on the basis of a valence of −2. Finally, the rate of energy dissipation through such a circuit is equal to the product of current and of the potential of 36,068 J./mole. These notions can be extended to potential cycles involving ionized species with more than two charges.

3.5.2 The Most Efficient Circuit

The most parsimonious extension of the model to reconcile the multiple potential circuits of a multi-protic proton shuttle stems from the postulate that the circuit through which energy will be dissipated at the highest rate will be favored at the exclusion of all others (postulate of the maximally efficient circuit). The notion that the proton shuttle cycle can be reduced to an electrical circuit should not be taken to imply that neutral and ionized molecules diffuse along a physical conduit as in a strict electrical sense. Along this line, the existence of multiple possible circuits should not be taken to be synonymous with multiple path options, or to be analogous to a parallel electrical circuit arrangement. Rather, under the initial premise that each iteration of a proton shuttle cycle involves the simultaneous exchange of neutral molecules for ionized molecules according to a strict one-to-one stoichiometry that does not alter steady-state distributions, there is no energetic or resistance-lowering advantage to the coupling of the neutral species to any ionized species other than the one with which the most efficient circuit can be formed. Evaluating the activity of a multi-protic compound therefore requires identifying the most energetically-efficient of its potential circuits.

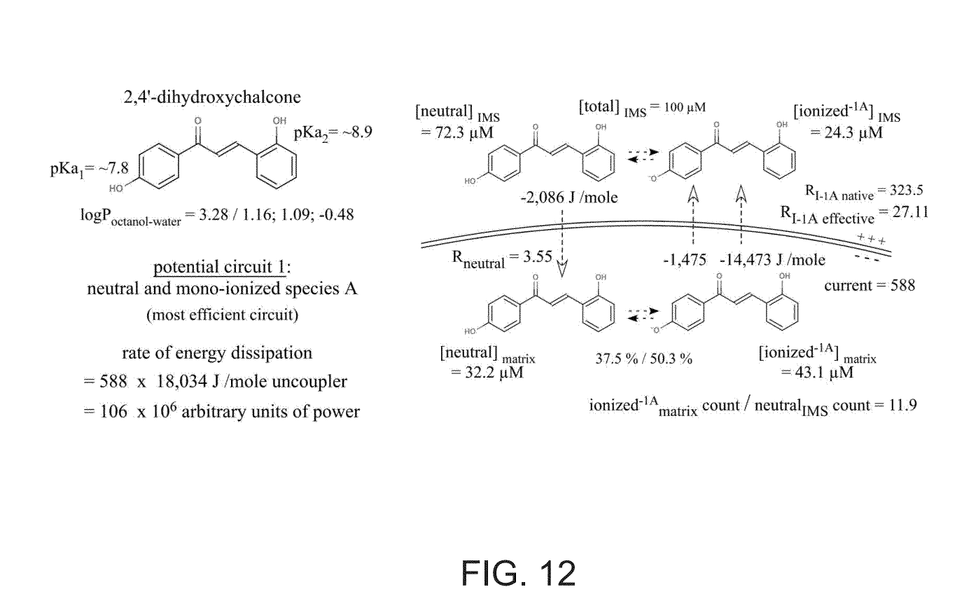

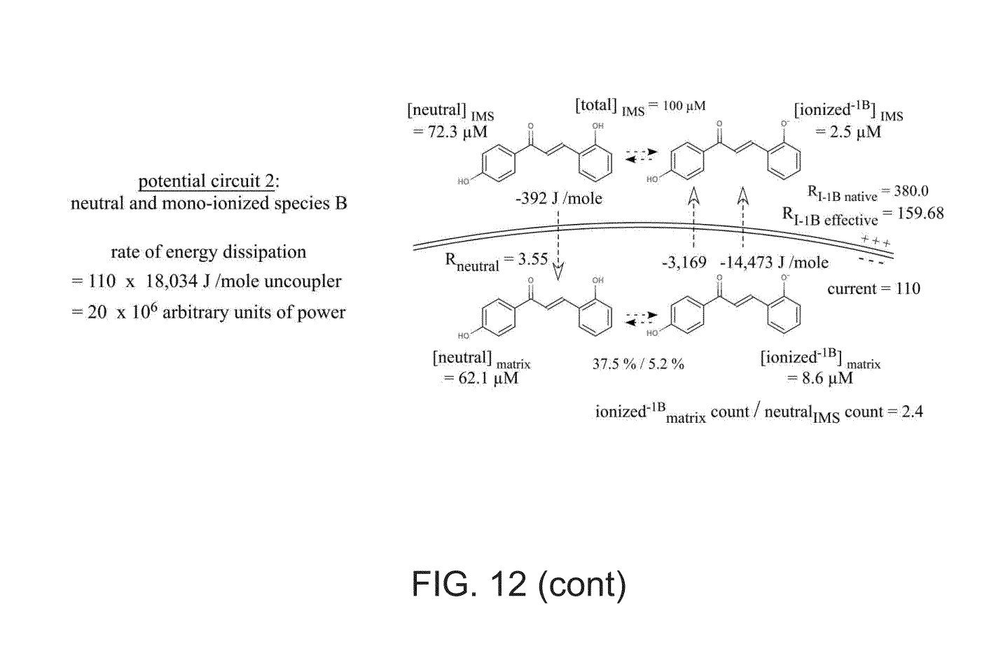

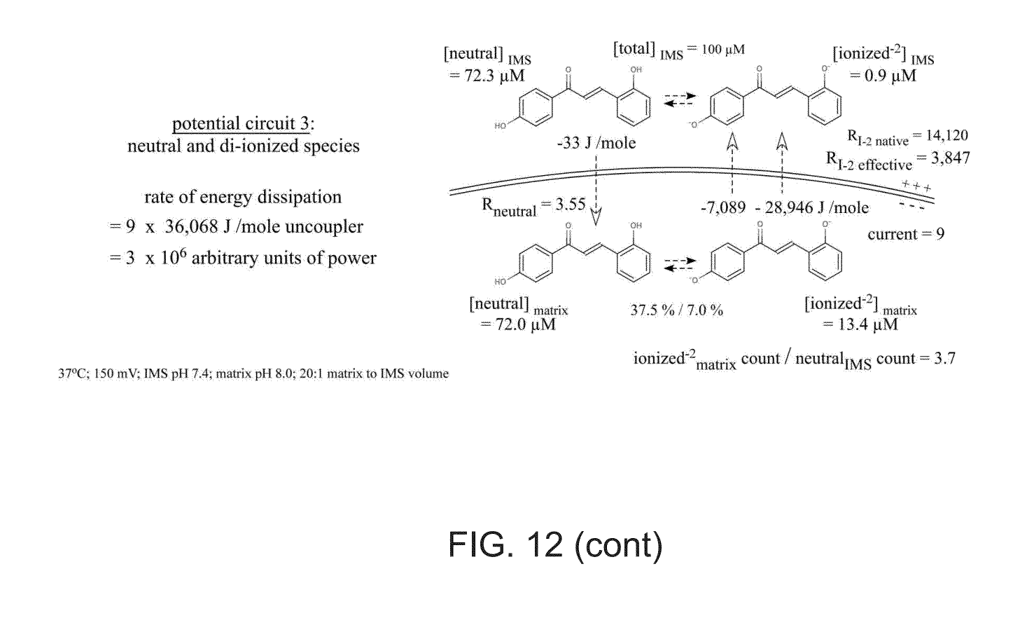

It should be appreciated that, as the ionized species of a given multi-protic compound differ from each other on the basis of resistance to diffusion, of distribution/concentration, and of valence, the ionized species with which the most efficient circuit can be formed is not necessarily the species that exhibits the lowest native resistance. From this, the most efficient circuit can be identified only by evaluating the potential rate of energy dissipation through each of the possible circuits. FIG. 12 summarizes calculations of steady-state conditions and activity for each of the three possible circuits of an example di-protic compound. Note that the magnitude of the resistance-lowering effect of asymmetrical molecular distribution is calculated independently for each circuit.

It follows from this that the activity of a multi-protic compound is nearly always inferior to that of a mono-protic counterpart (i.e., with identical pKa₍₁₎ and lipophilicity). The basis for this prediction is two-fold. First, additional hydroxyl groups detract from lipophilicity and must be compensated by lipophilic substitutions, thereby increasing molecular bulk. Second, and more importantly, as the molecular count of the most efficient ionized species of a multi-protic compound is necessarily less than the total count of all ionized species, this species tends to benefit from a smaller resistance-lowering effect than the sole ionized species of a mono-protic compound with the same pKa₍₁₎. This dilution effect is most obvious in the case of multi-protic compounds that exist as multiple mono-ionized species, with no multi-ionized species.

It also follows that when mono- and multi-ionized species exist at mitochondrial pH, the most efficient circuit is typically one composed of the neutral species and a mono-ionized species; the advantage of shuttling more than a single proton per cycle iteration (i.e., multiplication of the ΔG per mole uncoupler) is calculated, in most cases, to be completely offset by the important decrease in lipophilicity of a multi-ionized species relative to its mono-ionized counterparts (2 to 3 orders of magnitude for each additional ionization event). However, as modeled, the margin is sufficiently narrow in the case of di-ionized species that the most efficient circuit can be one composed of a di-ionized species if such a species is the most prevalent ionized species and/or benefits from a very large resistance-lowering effect of distribution. This is predicted to occur in 27 of the 169 multi-protic compounds considered in Sections 4.1 and 4.2, below, and is also predicted to be the case for the reference protein-assisted uncoupler S-13. Moreover, if the compound is specifically di-protic and its di-ionized species benefits from a near-maximal resistance-lowering effect thanks to optimal acid-dissociation properties, then it is possible that this multi-protic compound can outperform its mono-protic counterparts. By extension, the model predicts the most powerful proton shuttles to be di-protic compounds that shuttle two protons per cycle iteration. This is expanded in Section 4.4.2, below, with proposed examples.

3.6 Minor Variations of the Proton Shuttle Mechanism

Variations of the proton shuttle mechanism that relate to cationic compounds, basic compounds, or compounds that shuttle protons in oligomeric form can all be accommodated by the proposed theoretical framework.

3.6.1 Cationic Compounds as Proton Shuttles

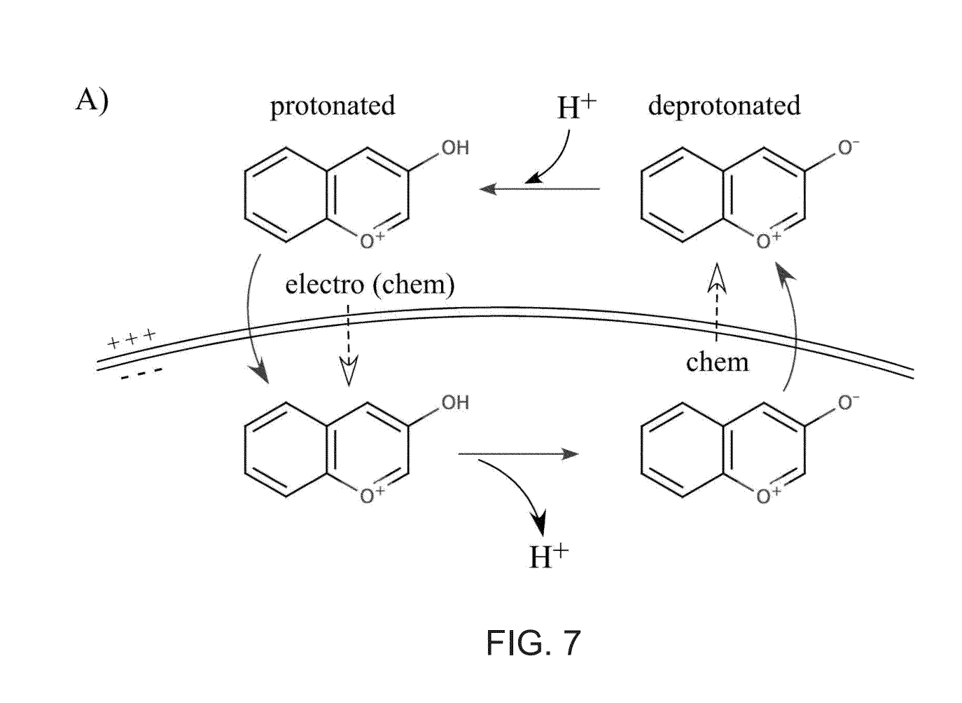

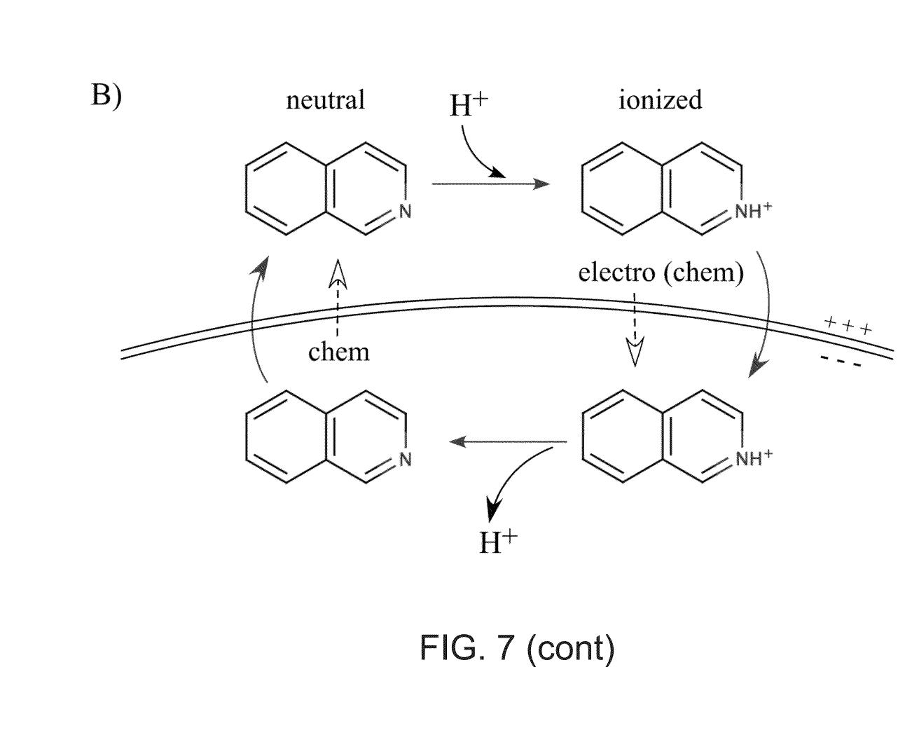

Up to this point, proton shuttles have been described as lipophilic weak acids that exist in both neutral and ionized forms within the compartments of the mitochondrion. In a minor variation of the proton shuttle mechanism, the shuttle can be a cation (FIG. 7A), positively charged rather than neutral when in protonated form, and electrically neutral (no net charge) rather than negatively charged when in (mono-) deprotonated form. As such, it is the protonated rather than the deprotonated species that is subject to the electrical gradient across the mitochondrial inner membrane, and proton shuttle molecules are driven into the matrix rather than out by this electrical force. Correspondingly, the deprotonated species diffuses along a chemical gradient from the matrix to the IMS. It should be noted that although the deprotonated species is electrically neutral, its negative charge nevertheless decreases its lipophilicity relative to the protonated species.

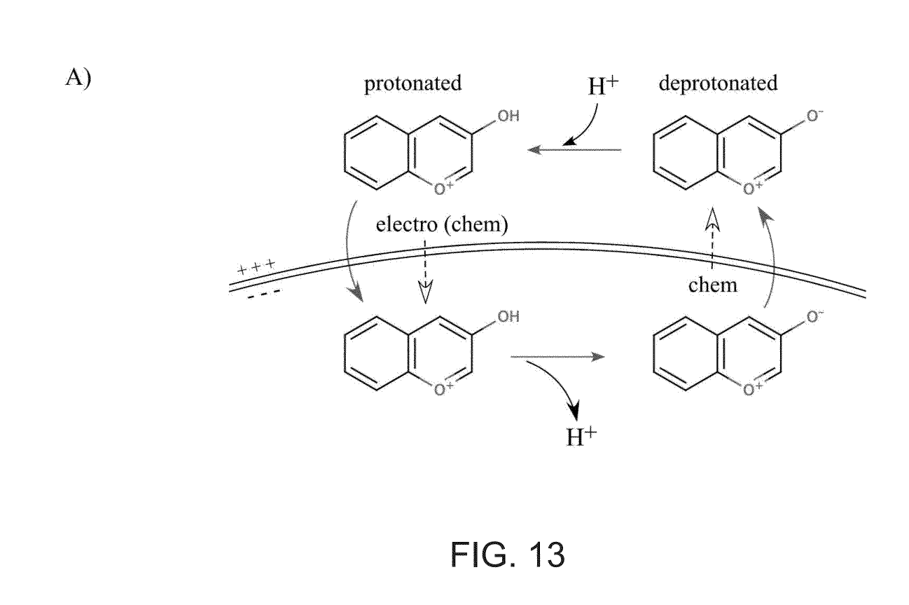

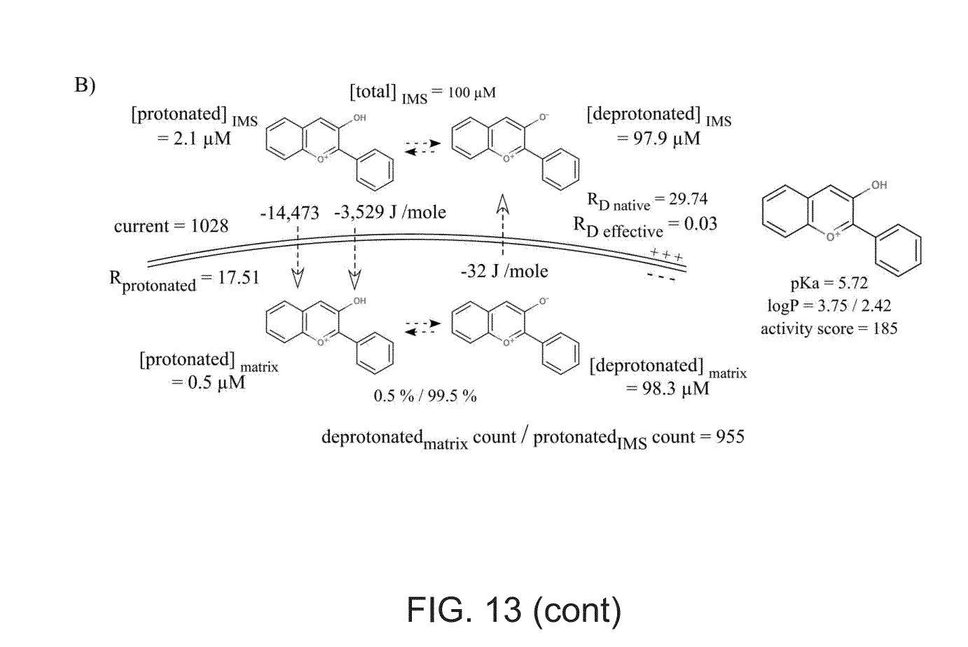

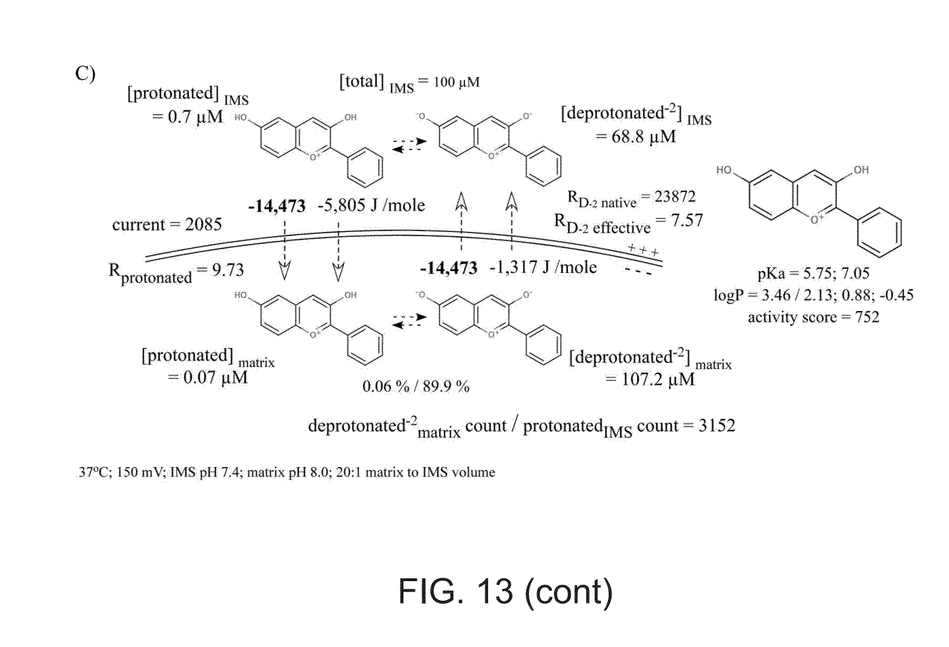

The framework developed for traditional non-ionic proton shuttles can be adapted to cationic compounds simply by accounting for the gain of a positive charge on the protonated side of the model and the corresponding net loss of a negative charge on the deprotonated side. FIG. 13A summarizes the calculations of steady-state conditions and activity for the cationic compound 3-hydroxyflavylium, derived from the flavylium ion backbone of the anthocyanidin class of flavonoids.

Like non-ionic proton shuttles, cationic proton shuttles can, of course, be mono-protic or multi-protic acids. As developed above for non-ionic compounds, assessment of the activity of a cationic multi-protic compound requires that all possible circuits be considered, including those that involve a species ionized at multiple sites and through which more than one proton can be shuttled per cycle iteration. However, whereas a mono-deprotonated species of a cationic proton shuttle is electrically neutral, a multi-deprotonated species exhibits a net negative charge. Therefore, circuits involving the protonated species and a species ionized at multiple sites are characterized by electrical diffusion gradients acting on both species; the positively charged species is driven into the matrix by the membrane potential while the negatively charged species is driven out. FIG. 13B summarizes the calculations of steady-state conditions and activity for the cationic di-protic compound 3,5-dihydroxyflavylium. Along this same line, it is an intriguing possibility that the involvement of the fully-protonated species is not essential and that a circuit might be composed of two deprotonated species: an electrically-neutral mono-deprotonated species and a multi-deprotonated species with a net negative charge.

It should be noted that naturally-occurring anthocyanidins typically contain several hydroxyl substituents with calculated pKa values on the order of, or below cellular pH, and are often expected to exist in a dozen or more ionized forms within the mitochondrion. As a result, the activity of such compounds can be difficult to predict. Moreover, anthocyanidins exhibit pH-dependent stability, and acid-dissociation behavior estimated on the basis of the uncorrected structure may not be representative of acid-dissociation behavior at mitochondrial pH.

3.6.2 Lipophilic Weak Bases as Proton Shuttles

The proton shuttle mechanism of uncoupling is most often associated with lipophilic weak acids. However, a lipophilic weak base can also shuttle protons (FIG. 7B) [9A, 24A]. This occurs through a simple reversal of the mechanism described for acids. Specifically, in a base, the species that carries a proton from the IMS to the matrix (i.e., the protonated species) is the positively charged ionized species rather than the neutral (deprotonated) species. Because of its positive charge, the ionized species is driven into the matrix rather than out by the electrical potential across the inner membrane. Diffusion of the protonated species into the matrix is therefore driven by an electrical gradient, and correspondingly, diffusion of the deprotonated species into the IMS occurs down a chemical gradient.

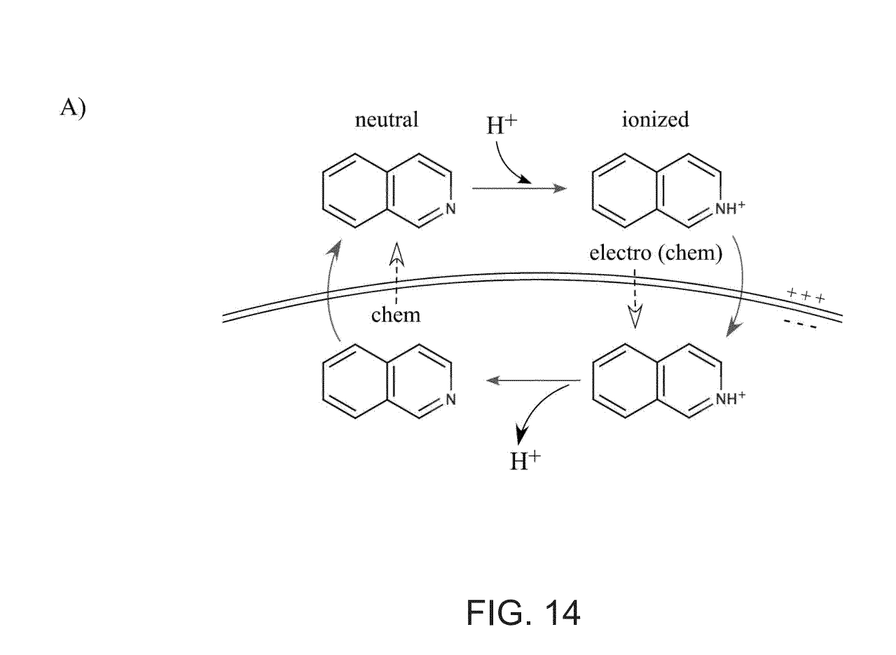

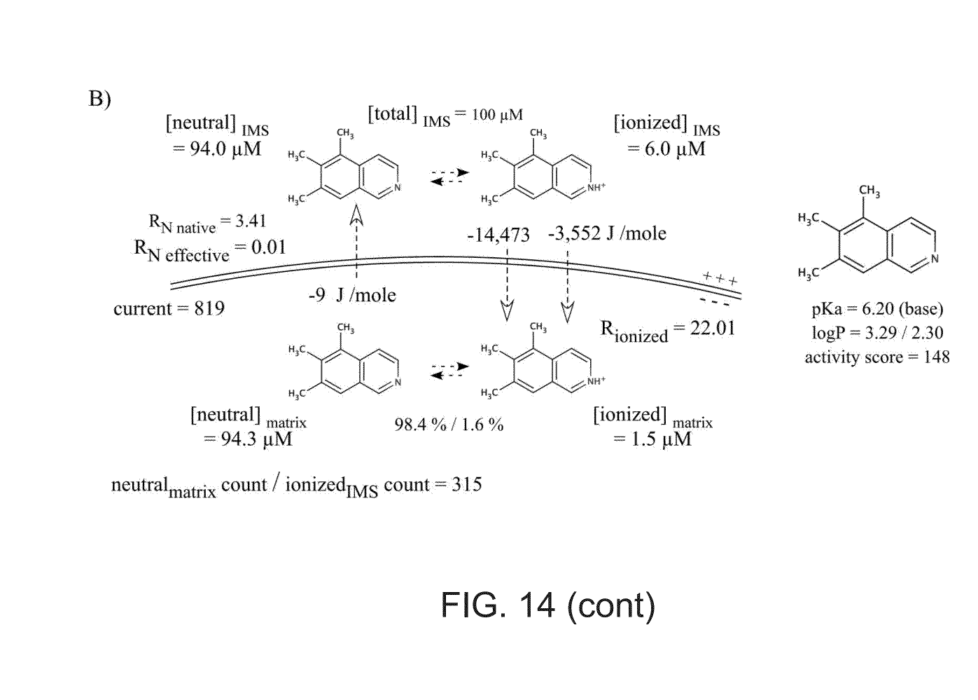

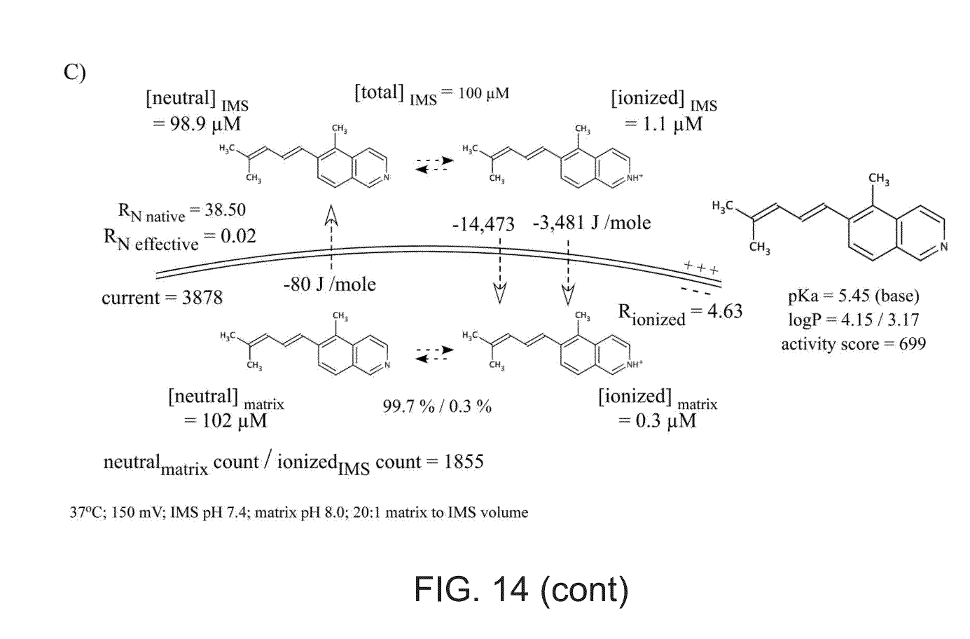

Basic uncouplers have not been as well studied as their acidic counterparts, likely because they occur less frequently. Nevertheless, there is no reason to expect that, given equivalent physicochemical properties, basic proton shuttles are any less effective than acidic proton shuttles. Indeed, the relationships governing resistance to diffusion can be expected to be identical for bases as for acids. Similarly, a resistance-lowering effect of distribution can be proposed for bases as for acids, albeit reversed such that activity increases with increasing values of basic pKa (up to the specified maximum of 11.4) rather than decreasing values. The framework developed for acidic proton shuttles can therefore readily be adapted to basic proton shuttles by reversing certain calculations. FIG. 14A summarizes the calculations of steady-state conditions and activity for a derivative of the naturally-occurring isoquinoline structure, optimized for low resistance to diffusion.

Note that the interaction of pKa and log P_{octanol-water} defines two activity spaces for bases as for acids. In the secondary activity space of bases, activity peaks as basic pKa decreases and the log P_{octanol-water} value of the ionized rather than the neutral species approaches the specified optimal value of 3.2. FIG. 14B summarizes the calculations of steady-state conditions and activity for a derivative of the isoquinoline structure designed to be excessively lipophilic in neutral form so as to take advantage of this secondary activity space.

Despite equivalent predicted efficacy, bases may be less attractive than acids from the point of view of rational design of optimized proton shuttles. One reason is that the chemical space of basic compounds is more limited than that of acids. Another is that basic compounds are not amenable to the fine-tuning of base-association behavior through resonance effects of substituents (see Section 4.2, below). Finally, nitrogenous compounds tend to be more difficult to metabolize than compounds composed of C, H, and O, and may therefore be less suited to applications where short-lived activity is desirable. Lipophilic weak base uncouplers will not be addressed further in this work.

3.6.3 Physicochemical Properties Enhanced Through Oligomerization

Oligomerization of a lipophilic compound can be expected to result in increased lipophilicity: in general, with each addition of a monomer, the overall value of log P_{octanol-water} is increased by an amount on the order of the value of the monomer itself. Thus, compounds with the propensity for oligomerization and that exhibit sub-optimal lipophilicity in monomeric form may exhibit near-optimal lipophilicity in dimeric or even oligomeric form. This may translate into increased activity if the gain in lipophilicity is not completely offset by the accompanying increase in molecular bulk and if acid-dissociation properties are not negatively affected by oligomerization. If a given compound exists as oligomers of various size, it can be expected that one n-mer will exhibit a more optimal degree of lipophilicity than all other forms. Moreover, each n-mer may have different isoforms, in which case it can be expected that one isoform will be more conducive to activity than others on the basis of geometric considerations and/or of acid-dissociation behavior.

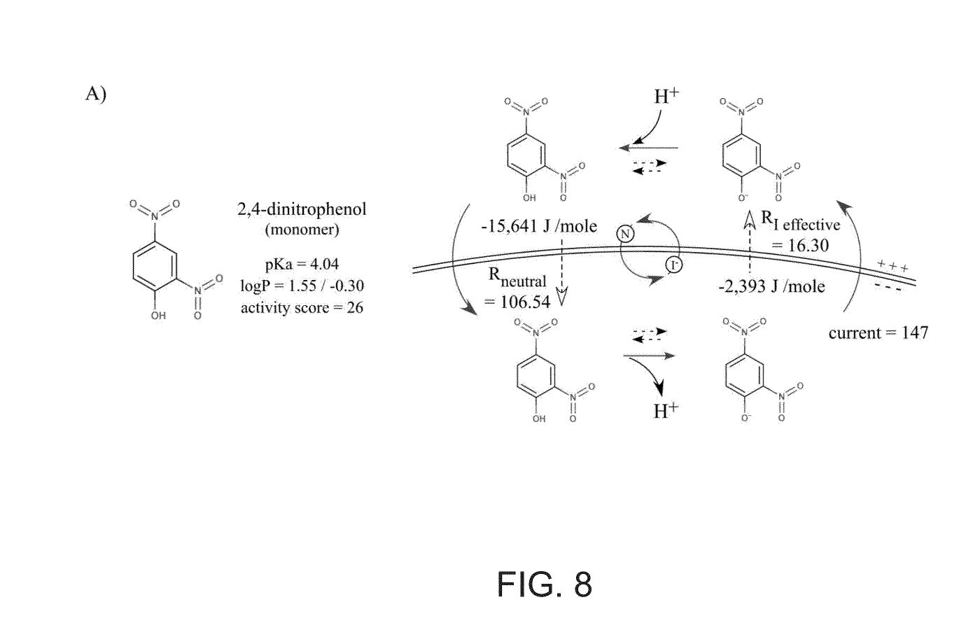

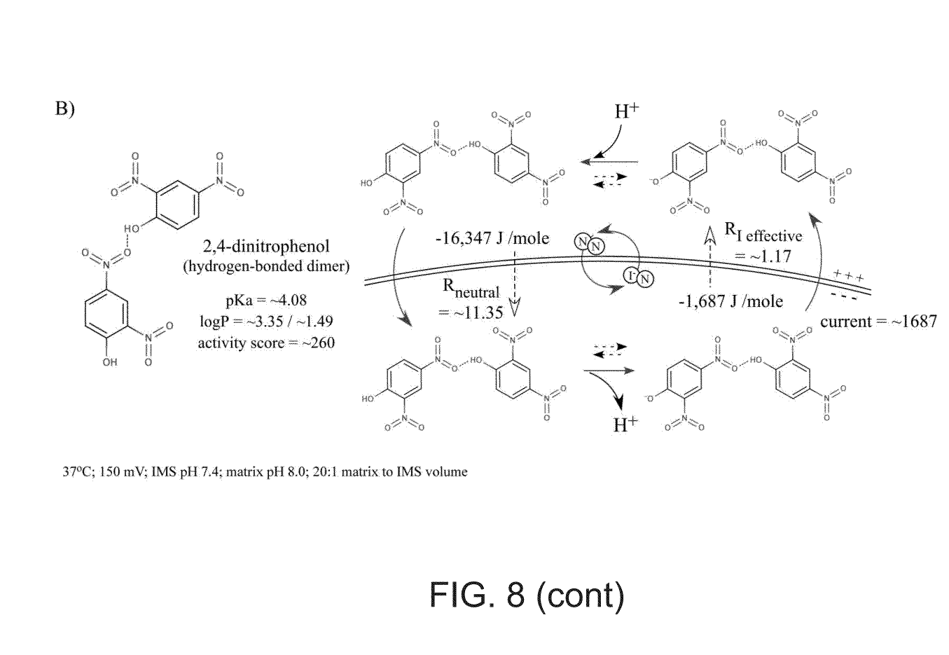

This special case of proton shuttle uncoupling is exemplified by the reference uncoupler 2,4-dinitrophenol (FIG. 8), notwithstanding that the activity of this compound is believed to be mediated to some degree by interaction with the adenine nucleotide translocase [2A]. 2,4-dinitrophenol is characterized by small size, optimal pKa of approximately 4.0, and extensive charge delocalization (reduction of 1.85 in log P octanol-water upon ionization). However, its log P_{octanol-water} value of 1.55 is incompatible with a high rate of diffusion. Instead, activity of this compound can be attributed to its dimeric form, presumably the result of a propensity for strong hydrogen bonding between a nitro group of one monomer and the hydroxyl group of another. In dimeric form, the log P_{octanol-water} value is approximately double that of the monomer, as the number of substructures that contribute to lipophilicity and the number of substructures that contribute to hydrophilicity are both approximately doubled. It is noteworthy that two dimer isoforms are possible. Both are predicted to be better proton shuttles than the monomer since their increase in molecular bulk only partially cancels out their gain in lipophilicity, and net resistance to diffusion is therefore decreased. However, one dimer is predicted to retain a near-optimal pKa of 4.1, whereas the other is predicted to exhibit a one unit increase in pKa. The first dimer is therefore predicted to shuttle protons with 10-fold more efficiency than the monomer, whereas the second is predicted to exhibit only 2-fold more activity. Calculations of steady-state conditions and activity for the monomer and for the more efficient of the two dimers are summarized in FIG. 8.

In some cases, oligomerization may also contribute to uncoupling activity by modulating acid-dissociation behavior. For example, salicylic acid, whose pKa₍₁₎ is calculated to be 2.8 and therefore incompatible with proton shuttle activity, dimerizes at high concentrations through intermolecular hydrogen bonding between carboxyl groups. As the hydrogen-bonded carboxyl group is no longer ionizable, the higher pKa hydroxyl substituent instead becomes the most readily ionizable site. Consequently, uncoupling activity is conferred to the dimer. Other carboxylic acids may behave in this way.

When assessing a compound with a propensity for spontaneous oligomerization, it is possible to identify the form of this compound that is most conducive to proton shuttle activity, as above, by comparing the predicted activity of the monomeric form to that of potential oligomeric forms of increasing size. However, it is not possible to predict that compound's overall activity without knowledge of the relative distribution of all forms of the compound present in the mitochondrion. If such knowledge is available, then overall activity can be taken to be equal to the sum of the activity of each form present, weighted according to respective effective concentration. For example, if under physiological conditions 2,4-dinitrophenol exists in dimer form only, and the two possible dimers are equally probable (i.e., exhibiting the same change in free energy from dimerization), then it can be proposed that two distinct chemical species contribute additively to the activity of the compound and that the effective concentration of each is ¼ of the reference (monomeric form) concentration; activity for the compound is then taken to be ¼ of the score of dimer 1 plus ¼ of the score of dimer 2. Without knowledge of distribution, it is only possible to predict that activity of 2,4-dinitrophenol is less than or equal to ½ of the score of the most efficient dimer.

It should be noted that if a compound's propensity for oligomerization is not recognized and taken into consideration, then predictions of activity based on the monomeric form may be erroneous. Along this line, it may be appropriate to consider not only the propensity for oligomerization through hydrogen bonding, but also that for spontaneous oxidative coupling, such as might be expected of the building blocks of tannins and procyanidins, for example.

Finally, the converse of the notion of compounds that shuttle protons more efficiently in oligomeric rather than in monomeric form is that of compounds whose metabolites are more efficient proton shuttles than the parent compound. From this, potential metabolites with near-optimal lipophilicity should be considered whenever attempting to predict the activity of compounds with supraoptimal lipophilicity.

4. Results

The theoretical framework developed in Section 3 has been mathematically implemented in the form of a spreadsheet (This proposed implemenation is available at doi: 10.1016/j.jtbi.2012.02.032). As in preceding sections, all calculations are based on the following conditions: 37° C.; IMS pH 7.4; matrix pH 8.0; 150 mV transmembrane potential; 20:1 matrix to IMS volume ratio. Driving potential is therefore taken to be 18,034 J./mole as described in Section 3.1. However, because resistance to diffusion is expressed relatively as described in Section 3.3, current must be expressed as arbitrary units of current, and activity as arbitrary units of rate of energy dissipation. In the present section, the predictive power of this model of proton shuttle activity is gauged and the model is applied to the screening of a library of naturally-occurring compounds, to the identification of chemical templates conducive to activity, and to the design of derivatives of these templates optimized for maximal activity. Patterns of speciation, log P_{octanol-water} values, and geometric parameters for all compounds analyzed are estimated using a commercial chemoinformatics package, as described in Section 2.1. All predictors, calculations, and results are recorded in the Supplemental Data File.

4.1 Evaluation of Model Predictive Power: Comparison of Predicted to Measured Activity

Validity of the theoretical framework was addressed by assessing the predictive power of its proposed mathematical implementation. Specifically, predicted proton shuttle activity was compared to uncoupling activity measured directly in isolated mitochondria under standardized conditions for a chemically-diverse test set of 48 naturally-occurring phenolic compounds. This large test set is the product of the recent screening of uncoupling activity in flavonoids (including flavones, isoflavones, flavonols, flavanols, and flavanones) and related compounds, including chalconoids, anthraquinones, stilbenoids, cinnamates, and simple phenolic compounds [12A].

The test set included nine compounds characterized as having significant uncoupling activity, with an U₅₀ (concentration at which 50% uncoupling is induced) as low as 10 μM. These span a range of calculated pKa₍₁₎ of 4.5 to 7.9, and exhibit a highly variable number of ionized species at mitochondrial pH, ranging from 1 to 11. Based on their structure, their relatively weak uncoupling activity, and their steep dose-response relationship [12A], it is likely that their activity is entirely attributable to the Mitchellian proton shuttle mechanism. However, potentiation of activity through protein-interaction has not been ruled out by testing sensitivity to carboxyatractylate, 6-ketocholestanol, or cyclosporin A. These compounds are described in Table 1A. As annotated in the table, five of these nine compounds have also been identified by others as exhibiting uncoupling activity [23A, 25A-27A]. The balance of the test set consisted of less active or inactive phenolic compounds of the same classes represented by these nine, as well as of related classes. The low activity of these compounds may be attributed to a variety of reasons: insufficient lipophilicity in general; poor charge delocalization/insufficient lipophilicity of the ionized species; geometric considerations unfavorable to efficient diffusion, possibly combined with excessive lipophilicity; pKa₍₁₎ close to cellular pH; or absence of an ionizable group, as in the case of four completely inactive class parent compounds. Although inclusion of a large number of weakly active compounds results in a distribution bias, the test set is particularly well-suited to gauging the model's power to discriminate active compounds from poorly active or inactive ones. All 48 test set compounds are described in Supplemental Data Table 1. All are composed exclusively of C, H, and O. It should be noted that although the compounds tested span an important range of structure, the test set did not include cationic compounds (addressed in Section 3.6.1), such as anthocyanidins, or compounds that fall into the second of the two activity spaces described in Section 3.4. Finally, the test set did not include highly active reference compounds (e.g., arylhydrazones), as such compounds are invariably protein-assisted uncouplers and therefore inappropriate for assessing the ability to predict proton shuttle activity.

Predicted proton shuttle activity expressed in relative terms was compared to uncoupling activity measured by oxygraphy in isolated rat liver mitochondria at a test compound concentration of 100 μM. As described in detail in the original screening study, activity was assessed as the xenobiotic-induced stimulation of the rate of basal succinate-supported oxygen consumption (i.e., in the absence of ADP; state 4 respiration), expressed relative to the increase stimulated by a saturating amount of ADP (i.e., state 3 respiration) in vehicle-treated mitochondria of the same preparation; 0% uncoupling therefore indicates no increase in basal stimulation, whereas 100% uncoupling indicates basal oxygen consumption increased to the rate of state 3 respiration. (In this system, the state 3/state 4 coupling ratio is typically between 4.5 and 5, and uncoupled oxygen consumption can exceed the rate of state 3 respiration by up to 30%). Inclusion of weakly active and inactive compounds in the test set dictated that measured activity is expressed as the magnitude of uncoupling at a standardized concentration, rather than the concentration corresponding to a standardized magnitude of effect (e.g., U₅₀). This single concentration approach is valid so long as there is no saturation of the uncoupling effect at the test concentration; a concentration of 100 μM was deemed optimal for these purposes, falling close to the upper limit of the linear portion of the dose-response relationship of the most active compounds of the test set [12A], and therefore providing maximum resolution for measuring activity in weakly active compounds. A drawback to this approach, however, is the possibility of underestimating the measurement of uncoupling activity in special cases of concurrent inhibition of oxidative phosphorylation between complexes II and IV of the electron transport chain (where a bell-shaped dose-response relationship is exhibited rather than the saturating relationship typical of uncouplers)[12A]. This may be addressed by extrapolating activity from measurements taken at lower concentrations that fall within the linear portion of the dose-response relationship. However, because such concurrent inhibition was suspected in only a few cases (e.g., formononetin, butein), this correction was not attempted.

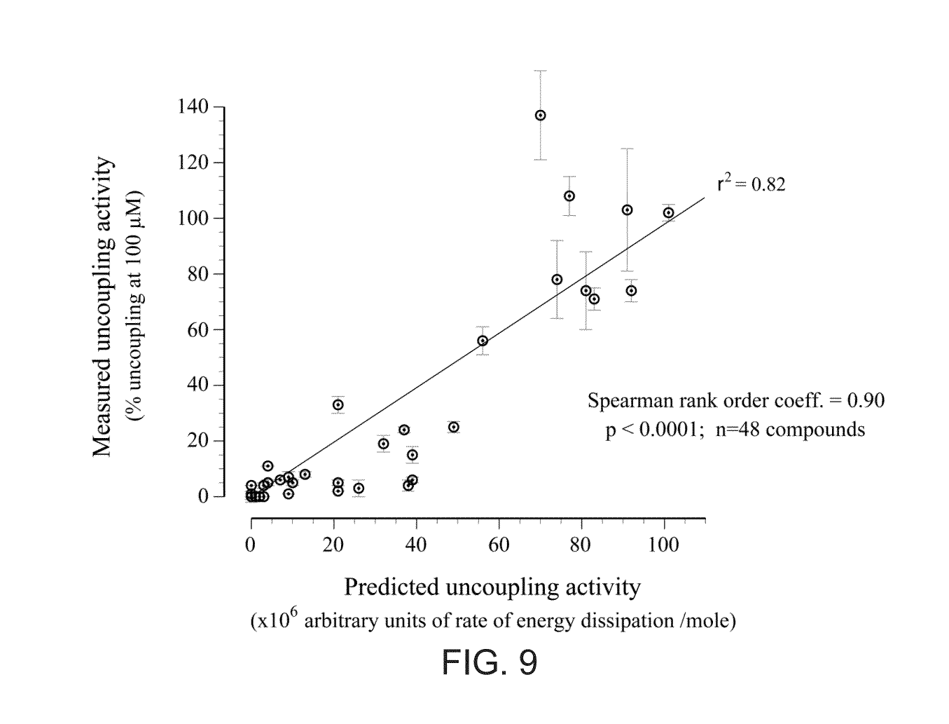

Satisfactory predictive power was indicated by a Spearman rank-order correlation coefficient of 0.90 (FIG. 9). The model successfully resolved the nine most active compounds from the rest of the test set, in spite of the considerable structural diversity and wide range of acid-dissociation behavior exhibited by these compounds. Moreover, the weak activity or absence of activity in other compounds could be attributed in all cases to unfavorable lipophilicity, acid-dissociation behavior, molecular size and/or shape, or a combination of these explanations. In only a few cases was predicted activity overestimated relative to measured activity. This best fit was obtained with the optimum degree of lipophilicity for diffusion set to a log P_{octanol-water} value of 3.2 (Section 3.3.1).

It should be noted that the present assessment of predictive power cannot distinguish error in the estimation of permeability behavior (Section 3.3) or error in structure-based calculations of pKa and log P_{octanol-water} values (Section 2.1) from error in the theoretical framework of the model. However, the use of calculated rather than experimentally-determined physicochemical parameter values surely contributes to overall error of fit. Indeed, acid-dissociation constants can be difficult to accurately estimate in multi-protic compounds characterized by fused carbocyclic and heterocyclic rings and in which the electronic effects of several substructures must be considered; comparisons of calculated to measured values, when available, suggest that the estimation of pKa can in some instances be off by 1 or more pH units. Underestimation of pKa may explain the few instances of overestimated activity, namely involving compounds of the stilbenoid class and of the flavanone subclass of flavonoids. While log P_{octanol-water} calculations are generally robust, the importance of this parameter to the prediction of activity is such that even small errors on the order of half a unit or less can significantly impact the accuracy of prediction. The observed tendency for increasing spread at higher activities may be attributable to a decreasing tolerance for error in the calculation of physicochemical predictors as activity increases. It may be appropriate to conduct additional comparisons of predicted to measured activity with compounds whose physicochemical properties have been experimentally measured in order to remove error associated with the structure-based calculations. However, assessing the validity of the theoretical framework of the model independently from that of the estimation of permeability behavior from physicochemical parameters is likely better performed using alternate experimental approaches, such as those proposed in Section 5, below.

The reference uncoupler 2,4-dinitrophenol, originally included in the test set as a positive control [12A], was excluded from the correlational analysis since the 100 μM test concentration falls slightly outside the linear portion of its dose-response relationship [12A], its activity is believed to be at least in small part attributable to a non-Mitchellian mechanism [2A], and calculation of its proton shuttle activity requires special assumptions (described in Section 3.6.3 and FIG. 8). It is noteworthy that inclusion of 2,4-dinitrophenol based on ½ of the activity predicted of its most efficient dimer in FIG. 8 (resulting predicted activity 130×10⁶ a.u.; measured activity 143%; U₅₀^{˜}10 μM) would improve slightly the fit of the overall relationship.

Although the model is designed to predict activity in relative terms, conclusions concerning absolute activity can nevertheless be drawn from the relationships illustrated in FIG. 5 and FIG. 9. It is first necessary to appreciate that as a consequence of expressing predictors of permeability relative to a specified optimal value on open-ended scales, only compounds that exhibit pKa outside of the specified limits of compatibility can be considered as having zero activity; conversely, all (mono-protic) compounds exhibiting pKa between 4.0 and 11.4 fall within the non-zero activity space. For practical purposes, however, it is useful to define a minimal threshold below which activity is unlikely to be detectable by respirometry or other means at concentrations below 100 μM; based on the relationship between predicted and measured activity, a value of 10×10⁶ a.u. is proposed as such a threshold. This threshold is indicated on the left side of FIG. 5 by a dotted line. Based also on these results, the value of 100×10⁶ a.u. can be taken to correspond to an U₅₀ on the order of 10 μM. Then, assuming a 1:1 linear relationship between concentration and activity, the maximal predicted activity plotted in FIG. 5 (peak value of 1074×10⁶ a.u. calculated for compounds composed exclusively of C, H, and O, and specifying a 2.1 unit decrease in log P_{octanol-water} incurred upon ionization) can conservatively be taken to correspond to a best U₅₀ of approximately 1 μM. Note that this maximum can be slightly bettered if a more optimal decrease in lipophilicity incurred upon ionization is specified (e.g., peak value of 1208×10⁶ a.u. for a decrease of 1.33 units). However, presence of halogen atoms or nitrogenous substituents is not expected to significantly increase maximal predicted activity. Examples of synthetic compounds designed to exhibit near-maximal activity are proposed in Section 4.4, below.

4.2 In-Silico Screening for Proton Shuttle Activity

A primary application of the present work is the in-silico screening of xenobiotics for proton shuttle activity. Persistent uncouplers of oxidative phosphorylation are of relevance to environmental toxicology, and in-silico screening can be useful to identify such compounds. Given that a wide variety of botanical products exhibit uncoupling activity, in-silico screening can also be of use to the assessment of the innocuity of natural health products and their constituents. Along similar lines, botanical compounds with uncoupling activity are often the basis of the anti-hyperglycemic effects of traditional treatments for diabetes [16A, 17A] and the ability to evaluate the activity of constituents of these products as they are identified can facilitate the process of isolating active principles.

To demonstrate its use as a tool for identifying proton shuttles with significant activity, the model was applied to the prediction of the activity of 252 naturally-occurring compounds. In keeping with the notion that significant uncoupling activity does not require the strongly electron-withdrawing groups traditionally associated with uncouplers, all compounds screened are composed exclusively of C, H, and O. These span several structurally-diverse chemical classes, including all classes and subclasses represented in the test set of Section 4.1 and several others closely related.

Specifying 45×10⁶ a.u. as a threshold, the screening tentatively identified 62 phytochemicals as proton shuttles with activity of physiological significance. These are described in Table 1B. While uncoupling activity has only previously been ascribed to seven of these known compounds [26A, 28A-32A], as annotated in the table, the majority of the 62 compounds belong to the same classes as the most active compounds tested in Section 4.1, and their identification is therefore generally made with a high degree of confidence. As in Section 4.1, many of these are multi-protic compounds, and, in several cases, activity is predicted to be mediated by the combination of the neutral species and a di-ionized species rather than a mono-ionized species (Section 3.5.2). Some identifications, however, should be considered highly tentative. These include the five highly lipophilic compounds artelastin, bolusanthol C, millewanin A, 6,8-diprenylgenistein, and 3′,5′-diprenylgenistein, as well as the fifteen compounds belonging to the flavylium cation (anthocyanidin) subclass of flavonoids. The first five fall into the second of the two activity spaces described in Section 3.4. Moreover, their activity is predicted to be mediated by a di-ionized species, but the calculated margin of difference between the most efficient di-ionized circuit and the most efficient mono-ionized circuit is uncharacteristically large. Flavylium cations can exhibit pH-dependent instability. Therefore, acid-dissociation behavior estimated on the basis of their generic structure may not be representative of their behavior at mitochondrial pH. In support of this, there exist no reports of the uncoupling activity of anthocyanidins in animal mitochondria.

The most active of the non-flavylium compounds of interest were calculated to exhibit activity on the order of 150×10⁶ a.u. Assuming linearity, this represents 50% more activity than the best compounds tested in Section 4.1, and therefore corresponds to an U₅₀ of below 10 μM. Actives of the flavylium cation subclass of flavonoids may even surpass this level of activity. The compounds described in Tables 1A and 1B are therefore proposed to be the most powerful naturally-occurring proton shuttles identified to date.

4.3 Identification of Naturally-Occurring Templates Conducive to Activity

The 48 phenolic compounds of Section 4.1 represent several broad phytochemical families and span a wide range of chemical structures. However, the most active among these are hydroxy-substituted members of only three structurally-distinct groups: the chalconoid class, the flavone/isoflavone subclass of flavonoids, and the anthraquinone class. While the library screened in Section 4.2 includes over 250 compounds and spans a considerably broader range of chemical structures, the compounds identified as being of interest also mostly arise from these same groups. This suggests that a handful of core structures or chemical templates are particularly conducive to proton shuttle activity, imparting some key physicochemical characteristics to their derivatives, and that these may be the basis of the majority of naturally-occurring proton shuttles. Such templates may also be appropriate starting points for the rational design of optimized synthetic derivatives (Section 4.4, below).

Inspection of the compounds summarized in Tables 1A and 1B reveals that their activity can be ascribed to very narrow subclasses: active chalconoids are hydroxyl-substituted at the 4′ position; active (iso)flavones are either 7-hydroxy(iso)flavones or 3-hydroxyflavones (i.e., flavonols); active anthraquinones are hydroxyl-substituted at position 2. The main distinguishing feature of these four core structures is proposed to be a pKa near or below mitochondrial pH, and therefore more conducive to proton shuttle activity than the pKa of typical phenolics. Indeed, the defining hydroxyl substituent of each of these structures benefits from an ester-induced and position-specific pKa-lowering effect, resulting in a pKa that is markedly lower than that of hydroxyl substituents at other positions or that of phenol and simple phenolics (^{˜}10.0). Specifically, 4′-hydroxychalconoids, 7-hydroxy(iso)flavones, 2-hydroxyanthraquinones, and flavonols exhibit a pKa₍₁₎ on the order of 7.9, 7.5, 7.3, and 5.3, respectively.

These four structures also benefit from a log P_{octanol-water} value that is within half a unit of the specified optimal of 3.2: 4′-hydroxychalconoids, 7-hydroxyflavone/7-hydroxyisoflavone, 2-hydroxyanthraquinone, and flavonol respectively exhibit a neutral log P_{octanol-water} value of 3.6, 2.7, 2.6, and 2.7. A large number of substituted derivatives with near optimal lipophilicity can therefore be expected to occur naturally. Moreover, with the exception of flavonol, these structures exhibit extensive charge delocalization, with a decrease of log P_{octanol-water} on the order of 2.1 upon ionization of their defining hydroxyl substituent; flavonols undergo a much larger decrease of log P_{octanol-water}, on the order of 3.5, thereby offsetting their pKa advantage (Section 3.4) over the other structures. Finally, these four structures exhibit a planar conformation and linear shape, and therefore a small frontal area in relation to their mass (Section 3.2.1): respective minimal projection areas are 1.35, 1.78/1.52, 1.37, and 1.70 times that of phenol. Although similar properties can be observed in related structures, they must be combined with compatible acid-dissociation behavior in order to result in a chemical template particularly conducive to proton shuttle activity.

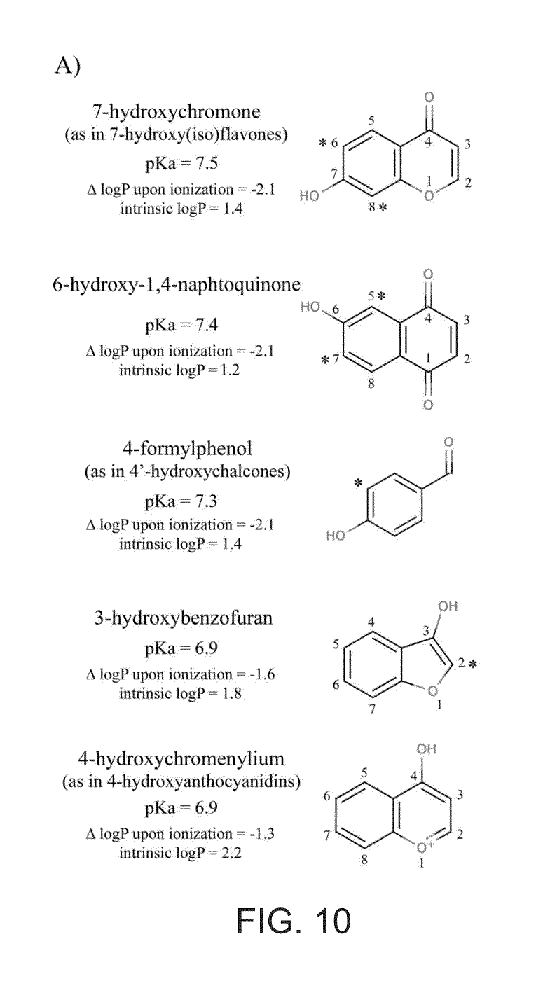

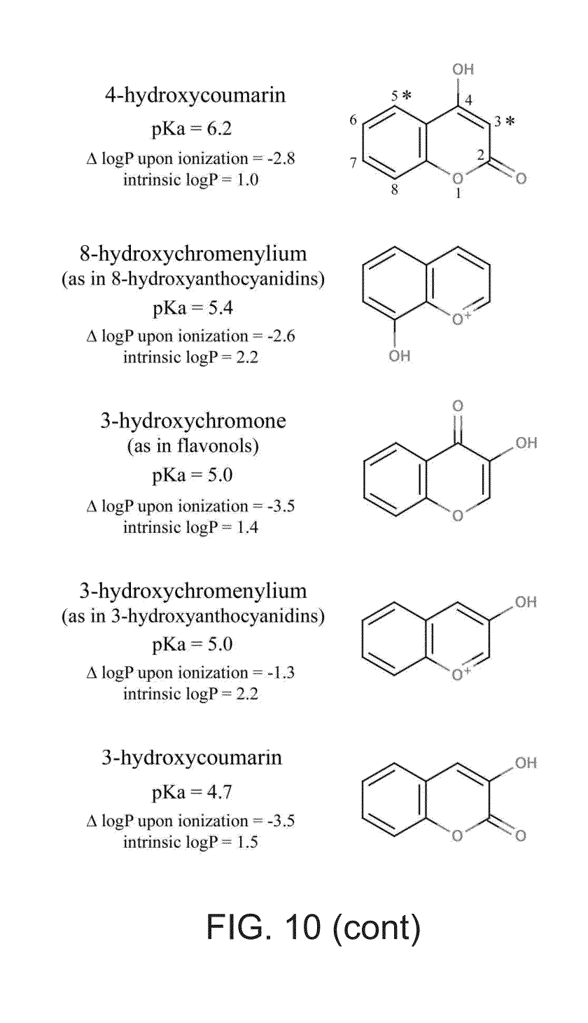

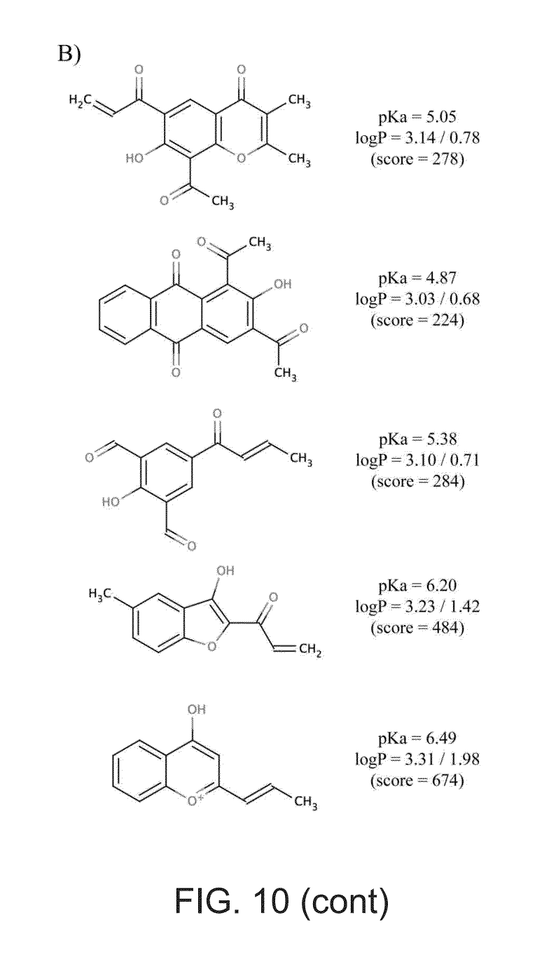

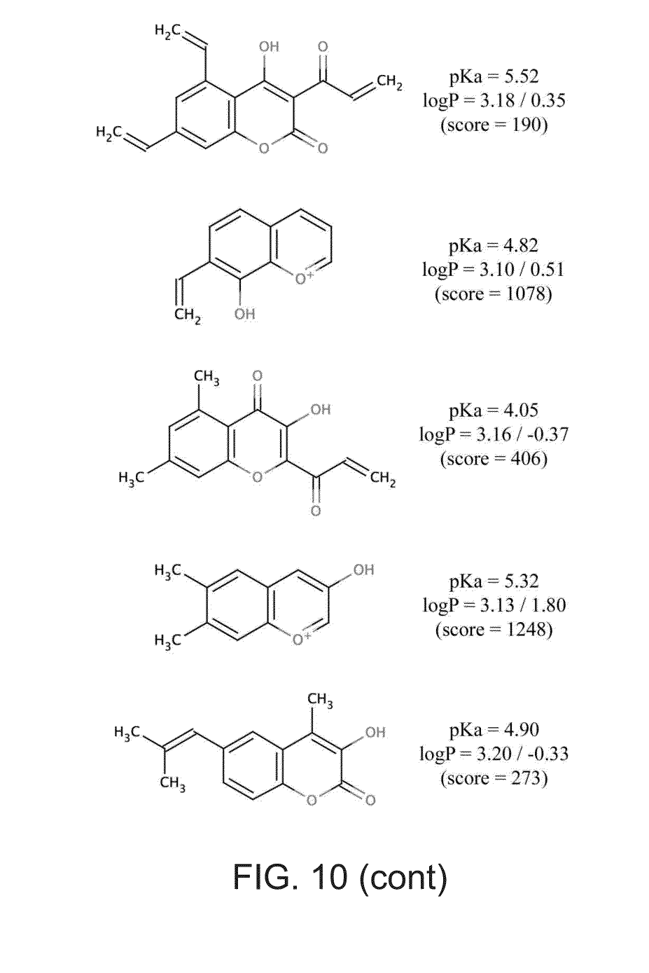

It is possible to further reduce these structures to their minimal core elements: the B-ring of (iso)flavone and flavonol can be considered a non-essential aryl substituent to a chromone core structure, either 7- or 3-hydroxy-substituted; 4′-hydroxychalcone can be similarly simplified to the 4-formylphenol core structure; 2-hydroxyanthraquinone can be simplified to 6-hydroxy-1,4-naphtoquinone (FIG. 10). The acid-dissociation behavior, propensity for extensive charge delocalization, and geometric considerations of these core elements are largely as described above for the more complex structures. Lipophilicity is, of course, significantly reduced, and as such, these core structures cannot be expected to exhibit proton shuttle activity without additional substitution. This is borne out by the observation that the compounds 7-hydroxychromone and para-acetylphenol tested in Section 4.1 exhibit no appreciable activity at 100 μM in isolated rat liver mitochondria (Supplemental Data Table 1).

Additional groups from which actives were tentatively identified in Section 4.2 include the benzofuran family and the flavylium cation (anthocyanidin) subclass of flavonoids. Applying a similar reductive approach to these groups, core elements conducive to proton shuttle activity can be identified as 3-hydroxybenzofuran (pKa₍₁₎ of 6.9; decrease in log P_{octanol-water} of 1.6 upon ionization; planar conformation) and the chromenylium cation, hydroxy-substituted at position 3, 4, or 8 (pKa₍₁₎ of 5.0, 6.9, 5.4, respectively; decrease in log P_{octanol-water} upon ionization of 1.3, 1.3, and 2.6, respectively; planar conformation)(FIG. 10). Of note, the hydroxychromenylium structures exhibit slightly greater neutral log P_{octanol-water} than other structures identified (with the exception of the larger 2-hydroxyanthraquinone), and therefore optimal lipophilicity for proton shuttle activity can be attained with less extensive substitution.

Although no actives were identified from the coumarin family in Section 4.2, the 3-hydroxy- and 4-hydroxycoumarin backbones can nevertheless also be considered core elements conducive to proton shuttle activity (FIG. 10). These are functionally analogous to the 3-hydroxy and 7-hydroxychromone structures above, whereby 3-hydroxycoumarin exhibits a particularly low pKa (below 5.0) but sub-optimal charge delocalization, whereas 4-hydroxycoumarin exhibits better charge delocalization but a higher pKa (6.2).

The notion of templates conducive to activity can be applied not only to acidic structures but also to basic ones. The naturally-occurring isoquinoline structure featured in FIG. 7B may be considered an example of such a basic template; although its low basic pKa of 5.3 is suboptimal for a basic proton shuttle, this structure exhibits remarkable charge delocalization, reflected by a decrease in log P_{octanol-water} of only 1 unit when in protonated form. Since the focus of this work is primarily on compounds composed exclusively of C, H and O, no attempt was made to identify other such basic templates.

Finally, it should be noted that structures with high pKa (the simplest being phenol) may also be considered conducive to activity if the second of the two activity spaces described in Section 3.4 is targeted rather than the primary space. However, such structures are of lesser interest than those described above for several reasons, not the least of which is that the greater lipophilicity (log P_{octanol-water} value on the order of 5) required by the second activity space carries with it an increased risk for toxicity.

4.4 Design of Synthetic Derivatives Optimized for Activity

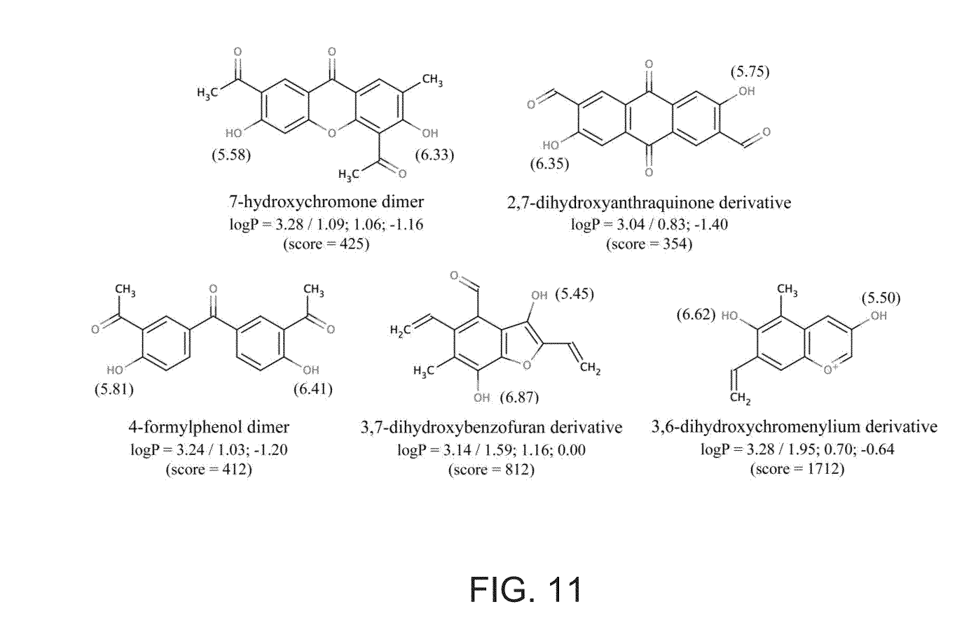

Ten chemical templates were identified, above, as particularly conducive to proton shuttle activity and as the basis of the activity of various naturally-occurring uncouplers (FIG. 10). These templates arise from six structurally-distinct classes, five of which are characterized by a fused ring system. The physicochemical constraints on proton shuttle activity detailed in the present work are such that the chemical space of naturally-occurring uncouplers with significant activity that is specified by these templates is clearly very large, encompassing perhaps thousands of compounds in addition to those listed in Tables 1A and 1B. From each of these templates can also be designed a large number of synthetic derivatives. General principles for the design of derivatives optimized for activity are here developed. Examples of such rationally-designed derivatives are provided in support (FIGS. 10 and 11; Supplemental Data Table 2), with the caveats that predictions of activity are based on estimations of physicochemical properties that are prone to error, that derivatives have not been tested, and that derivatives may exhibit unintended inhibitory activities at other sites within oxidative phosphorylation, as is common of lipophilic phenolic compounds. Additionally, it should be noted that the chromenylium structure is more reactive than phenol, benzofuran, chromone, coumarin, or anthraquinone, and that while its stability can be increased by substitutions at positions 2 or 4, hydroxychromenylium derivatives proposed in this section may be impossible to synthesize. Moreover, as with anthocyanidins, pH-dependent instability of chromenylium-based compounds may result in a higher pKa at mitochondrial pH than that estimated on the basis of generic structure.

Design principles are presented in the context of the most common type of proton shuttle, but can be adapted where needed so as to be applicable to basic compounds, compounds most effective in oligomeric form, or compounds that diffuse more easily in ionized than in neutral form.

4.4.1 Mono-Protic Derivatives

The design of optimized mono-protic derivatives of the templates described above is addressed first. In designing such derivatives, a first consideration is pKa. Given that proton shuttle activity is proposed to increase as pKa approaches its lower limit (arbitrarily defined as 4.0), then a derivative optimized for activity should exhibit a pKa as close to this limit as possible. The pKa of a given template can be decreased using electron-withdrawing substituents acting through inductive and/or resonance effects. Such substituents include halogen atoms and nitro and cyano groups. However, in keeping with the objective of identifying and designing easily-metabolized and low-persistence uncouplers, formyl and acetyl groups are favored instead. The magnitude of the pKa-lowering effect of an electron-withdrawing substituent is dependent on the position of the substituent relative to the ionization site. For each of the templates under consideration, positions at which such substituents exert maximal effect are indicated in FIG. 10 by an asterisk; at these positions, formyl and acetyl groups exert a pKa-lowering effect of 1 to 2 units. In templates with a relatively high pKa (i.e., 6.9 to 7.5), a larger effect may be desirable and can be achieved through the additive effect of two or even three such substituents. However, the advantage of optimizing pKa with more than one electron-withdrawing substituent must be balanced against geometric considerations of increased molecular bulk. Also, as a low pKa is proposed to confer a resistance-lowering effect upon the ionized species only, optimization of pKa will confer a greater benefit to templates that incur a larger decrease in lipophilicity upon ionization than to those that exhibit better charge delocalization.

As described in Section 4.3, the templates are insufficiently lipophilic to exhibit any appreciable proton shuttle activity without substitution. Formyl and acetyl pKa-lowering substituents can contribute a small concomitant increase in lipophilicity (halogen substituents significantly more so). However, achieving optimal log P_{octanol-water} for proton shuttle activity (proposed to be on the order of 3.2) generally requires the use of more lipophilic substituents. Such substituents are typically alkyls and alkenes, whose contribution to lipophilicity is largely proportional to chain length. Geometric considerations developed in Section 3.3.2 dictate that lipophilic substituents should be restricted to those that lie coplanar with the ring system of the templates, as this will contribute to minimizing frontal area. Thus methyl (+0.5 units of log P_{octanol-water}), ethenyl (+0.7), propen-1-yl (+1.1), 2-methylpropen-1-yl (+1.3), and 1-methylpropen-1-yl (+1.4) groups should be favored over isopropyl, allyl, dimethylallyl, prenyl, or cyclic substituents. Furthermore, if several positions are available for lipophilic substituents, then considerations of molecular shape further dictate that substituents be positioned in the long axis of the compound. Although alkyl substituents may exert a small electron-donating inductive effect when positioned in proximity to the ionizable group, geometric considerations will generally prevail over a small increase in pKa when choosing between substitution positions. The additive effect of a combination of lipophilic substituents may be required to achieve optimal lipophilicity. To achieve very fine-tuning of log P_{octanol-water} a slightly hydrophilic substituent such as a methoxy group (−0.2) may also be used in combination with a lipophilic substituent. In terms of geometry, a combination of small lipophilic substituents, such as two methyl groups, may in some cases be advantageous over a larger substituent of equivalent lipophilicity. Furthermore, substituents should not be placed at adjacent positions, if possible, as steric interactions may result, forcing distal atoms of the substituents out of plane and increasing the compound's frontal area. Finally, a lipophilic substituent can be positioned distal to the keto group of a pKa-lowering substituent rather than directly on the template's ring system if this confers a geometric or other advantage.

The final design consideration should be ease of chemical synthesis. Indeed, it may not be practical to implement all the design strategies outlined here if the final result is a compound poorly suited to synthesis. Rather, fine-tuning of activity should be weighed against increasing structural complexity.

An example of a mono-protic derivative optimized for high proton shuttle activity in respect to pKa, lipophilicity, and geometry (but not ease of synthesis) is illustrated to the right of each of the ten templates in FIG. 10. The predicted activity of each of these derivatives (indicated below the calculated values of pKa and log P_{octanol-water}) is likely representative of the maximal activity achievable from the respective template using only substituents composed of C, H, and O. From this, some templates may be said to be more conducive to activity than others. Namely, hydroxychromenylium cationic templates benefit from very extensive charge delocalization, higher intrinsic lipophilicity (baseline lipophilicity weighted by geometric considerations), and from absence of the interdependence between pKa and degree of charge delocalization that handicaps other templates. Indeed, other templates exhibit either good charge delocalization but tend to require the pKa-lowering effect of electron-withdrawing substituents, thereby incurring increased molecular bulk, or do not require electron-withdrawing substituents but exhibit a comparatively low degree of charge delocalization. Because of this tradeoff, the predicted activity of the most highly optimized derivatives from these templates is lower than the theoretical maximum calculated in FIG. 8 (on the order of 1100×10⁶ a.u. for compounds that exhibit a decrease in log P_{octanol-water} of 2.1 upon ionization and that are composed exclusively of C, H, and O). Using the reasoning described in Section 4.1, this corresponds to an U₅₀ of slightly below 5 μM for these derivatives. Activity of optimized derivatives of hydroxychromenylium templates, on the other hand, is closer to the theoretical maximum for these templates (on the order of 1400×10⁶ a.u. for compounds composed of C, H, and O, and that exhibit a decrease in log P_{octanol-water} of 1.33 upon ionization) and corresponds to an U₅₀ of approximately 1 μM.

In addition to the examples illustrated in FIG. 10, several other derivatives of each of the ten templates are proposed in Supplemental Data Table 2.

4.4.2 Di-Protic Derivatives

As treated under Section 3.4, multi-protic compounds are generally predicted to be less effective proton shuttles than their mono-ionized counterparts (that share a common structural template and that exhibit comparable resistance to diffusion of the neutral species); more extensive lipophilic substitution is required to offset the hydrophilic effect of additional hydroxyl groups, and the presence of multiple ionized species tends to reduce the magnitude of the resistance-lowering effect acting on any one species. Also as treated under Section 3.4, the model allows for the possibility that multi-protic proton shuttles can translocate two rather than one proton per cycle iteration in cases where a di-ionized species benefits from a large resistance-lowering effect of distribution. Consequently, in such cases the penalties associated with multi-protic compounds are offset by the thermodynamic advantage of translocating more than one proton per cycle iteration. In nearly all such cases, this offset is only partial. However, in cases of di-protic compounds exceptionally optimized such that the di-ionized species benefits from a near maximal resistance-lowering effect, the advantage can outweigh the penalties, and these di-ionized compounds can be more active than any of their mono-ionized counterparts. The following considers the design of such optimized di-protic derivatives from the templates described in Section 4.3.

In designing optimized di-protic derivatives, the same considerations of molecular geometry and of lipophilicity outlined above for mono-protic derivatives apply. However, considerations of acid-dissociation behavior differ slightly: the objective is no longer for ionized species in general to benefit from a near maximal resistance-lowering effect of distribution, but specifically for the di-ionized species to be favored. In other words, minimizing the proportion of the neutral species relative to the proportion of ionized species must be achieved in such a way as for the di-ionized species to be the most prevalent ionized species. Seeking to minimize only the pKa₍₁₎ of a di-protic compound results in a mitochondrial distribution skewed towards the corresponding mono-ionized species, possibly at the complete exclusion of the di-ionized species if pKa₍₂₎ is high. Instead, pKa₍₁₎ and pKa₍₂₎ must be minimized together. Since molecular distribution is the product of the interaction of both pKa₍₁₎ and pKa₍₂₎, it is more difficult to define a target value for pKa than in the case of mono-protic compounds. A value of 6.0 for both may be proposed as close to ideal, with 5.5 and 6.5 constituting a practical design target. Below this threshold, the neutral species can no longer be expected to exist at mitochondrial pH. As before, electron-withdrawing substituents can be used to fine-tune pKa. It is noteworthy that the molecular proportion of the di-ionized species can be further maximized relative to that of the mono-ionized species by designing symmetrical compounds such that ionization of either hydroxyl group results in a single mono-ionized species.

Given the vast number of permutations possible, it might be expected that several di-protic templates conducive to high proton shuttle activity could be derived from each of the mono-protic templates of Section 4.3. However, not all permutations are allowed: the existence of the neutral species at mitochondrial pH is precluded by the addition of a second hydroxyl group to some templates, or at certain positions in other templates. Moreover, it is essential that a template be capable of extensive delocalization of not one but two charges if the decrease in lipophilicity upon double ionization is to be minimized. The result is that only a handful of appropriate di-protic templates can be derived from the ten initial mono-protic templates. Furthermore, while each of these can produce a variety of highly optimized derivatives (several examples are proposed in Supplemental Data Table 2), derivatives predicted to be more active than their mono-protic counterpart of FIG. 10 can only be designed from five di-protic templates. An example from each is illustrated in FIG. 11.

These “super” proton shuttles, in which the advantage of translocating two protons per cycle more than offsets the intrinsic penalties of multi-protic compounds relative to mono-protic counterparts, are predicted to be up to 20-fold more potent than the best compounds tested in Section 4.1, or to exhibit an U₅₀ on the order of 500 nM; they are proposed to be representative of the most active proton shuttle uncouplers possible.

5. Discussion

The present work is intended as a predictive tool for identifying highly active proton shuttles and for guiding the design of such compounds for a variety of applications, including therapeutic uses. It is intended to have broad, class-independent chemical applicability. Rather than relating predictors of activity through a mathematical best-fit approach, this work is based on a theoretical framework that addresses several gaps in the decades-old understanding of the phenomenon. The mechanistic insight gleaned from this approach is likely the foremost contribution of the present work.

The theoretical framework is built on premises and hypotheses not formulated elsewhere. These are summarized as follows: 1) The exchange of one molecule of the neutral species of a proton shuttle for one molecule of the ionized species is coupled by mass action; 2) The sum of the Gibbs free energy for the diffusion of one molecule of the neutral species into the matrix and of the Gibbs free energy for the diffusion of one molecule of the (mono-) ionized species out of the matrix equals the Gibbs free energy for the translocation of one proton from the IMS to the matrix; 3) Within the constraints of IMS and matrix patterns of speciation, the system tends towards a steady-state distribution of the neutral and ionized species such that the flux of the neutral species into the matrix equals the flux of the ionized species out, thereby maximizing the rate of energy dissipation; 4) The effective resistance to diffusion of the ionized species is decreased when the matrix content of this species exceeds the IMS content of the neutral species; conversely, the effective resistance to diffusion of the neutral species is decreased when the IMS content of this species exceeds the matrix content of the ionized species; 5) In the case of multi-protic proton shuttles, the shuttling of protons involves a single ionized species at the exclusion of all other ionized species, and the combination of this species and the neutral species constitutes the circuit through which energy can be dissipated at the highest rate; when assessing rate of energy dissipation of potential circuits, points 1 to 4 must be adjusted, as required, to account for the translocation of more than a single proton per cycle iteration.

Points 1 to 3 allow for the calculation of steady-state neutral and ionized species concentrations and molecular distribution on either side of the inner membrane on the basis of pKa and of the permeability of the neutral species relative to that of the ionized species. Point 4 links pKa and lipophilicity, in accordance with long-standing empirical observations that these parameters are non-independent predictors of activity. Point 5 is presented as the most parsimonious extension of the model by which the activity of multi-protic uncouplers can be reconciled; in spite of their high occurrence, multi-protic compounds typically are not addressed by models of proton shuttle uncoupling. Finally, calculating the magnitude of the effect of point 4 on the basis of steady-state distribution, and taking resistance to diffusion to be equal to the inverse of permeability, the rate of energy dissipation, or uncoupling activity, is calculated as the square of the Gibbs free energy of proton translocation divided by the sum of the resistances to diffusion of the neutral and the ionized species. Given relative measures of permeability behavior, the activity of various compounds can thus be compared semi-quantitatively.

Conclusions that can be drawn from the theoretical framework and its proposed mathematical implementation (i.e., the model) include the following: 1) maximal uncoupling activity achievable through the unassisted proton shuttle mechanism (U₅₀ on the order of 1 μM) is greater than commonly believed; 2) proton shuttles can be divided into moderately lipophilic compounds with a pKa below mitochondrial pH, and highly lipophilic compounds with a pKa above mitochondrial pH; 3) mono-protic compounds tend to be more active than multi-protic counterparts exhibiting comparable permeability behavior; 4) it is often more efficient for a multi-protic compound to shuttle two protons per cycle iteration, rather than one, through a circuit composed of the neutral species and a di-ionized species; 5) in exceptional cases, a di-protic compound that shuttles two protons per cycle iteration can dissipate energy at a higher rate than any of its mono-protic counterparts; the most powerful proton shuttles are such compounds; 6) powerful proton shuttle activity is not dependent on the presence of nitro or cyano chemical groups or on halogenation, and can be achieved by compounds composed exclusively of C, H, and O.

The overall validity of this work is supported by a demonstration of good predictive power. Specifically, predicted relative activity was compared to experimentally-measured uncoupling for 48 structurally-diverse lipophilic weak acids exhibiting a wide range of activity, acid-dissociation behavior (pKa and number of ionization sites), and other physicochemical parameters. Broad chemical applicability of the model is also supported by its ability to reconcile special cases of the proton shuttle mechanism, including cations, bases, and hydrogen-bonded dimers (Section 3.6).

The degree of fit shown in FIG. 9 reflects not only the validity of the mechanistic framework but also that of the proposed method of estimating permeability behavior on the basis of octanol-water partitioning and molecular size and shape. Octanol-water partitioning is a well-established contributor to permeability behavior [21A] and predictor of uncoupling activity [8A] that can conveniently be estimated from compound structure with acceptable accuracy. It is here proposed that permeability behavior is directly related to octanol-water partitioning up to an optimal value of the partitioning coefficient, beyond which permeability behavior is inversely related to octanol-water partitioning. A log P_{octanol-water} value of 3.2 is proposed as this optimum on the basis of best-fit analysis. This value concords with empirical observations. Interestingly, this is below the values of 3.6 to 4.0 identified as optimal for some classes of protein-assisted uncouplers [4A, 18A], supporting the notion that effective interaction with a membrane protein may be achieved at the expense of some transmembrane diffusion efficiency. The relationship between partitioning behavior and permeability behavior used here can undoubtedly be refined, thereby improving overall predictive power. However, it is unlikely that the main conclusions of the present work would be significantly altered by specifying slope values that depart from unity, smoother transitions, or a plateau rather than a peak.

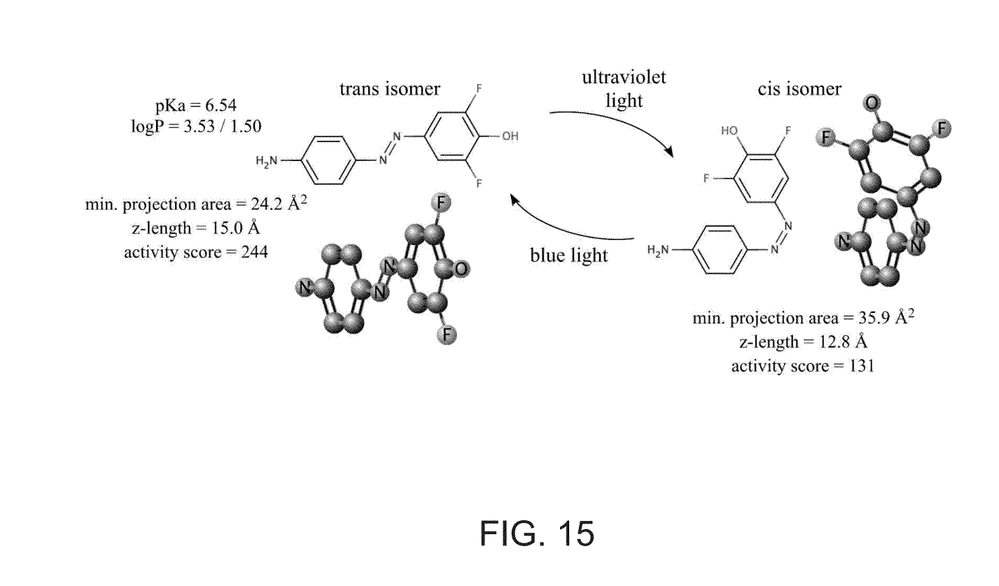

In addition to partitioning behavior, compound size and shape are also clear determinants of permeability behavior. In accordance with empirical observations that the most active proton shuttles tend to be planar and linear compounds [22A, 23A], it is here argued that below a molecular mass cut-off for membrane permeability on the order of 600 Da., shape is a more important predictor of activity than size alone. The geometric index proposed here, based on an assumption of anisotropic diffusion, sensitively captures compound shape and better resolves compounds than measures of mass or volume. As the weighting given to geometric considerations is significantly less than that given to octanol-water partitioning, refinements to this index of size and shape are not expected to have any impact on conclusions. Although its inclusion only slightly improves the overall predictive power of the model, this index or another such measure capable of resolving the range of compound shapes possible at any given compound mass, appears essential for reconciling lack of activity in compounds that otherwise exhibit appropriate physicochemical properties. The importance of such geometric considerations might be addressed in the future by comparing the activity of the stereoisomers of compounds with a chiral center. Alternatively, the activity of proton shuttles designed around a structure that exhibits cis-trans photoisomerization, such as azobenzene, might be modulated in real-time as the compounds toggle under ultraviolet illumination between a planar and linear conformation and a heavily kinked conformation (FIG. 15). Incidentally, it has been suggested that bulky substituents may be favorable to uncoupling activity if these are placed in such a way as to reduce solvent accessibility to the hydroxyl group; this is proposed to be the role of the two tert-butyl groups that flank the hydroxyl group of the highly potent phenolic protein-assisted uncoupler SF-6847 [8A]. However, while tert-butyl groups clearly contribute to lipophilicity (each one contributing 1.5 units of log P_{octanol-water}), they cannot be expected to affect the magnitude of decrease in lipophilicity incurred upon ionization. Therefore, although it is unclear whether the quality of interaction between SF-6847 and its protein target would be degraded, it is proposed that the permeability behavior of SF-6847 would be improved by replacing the tert-butyl groups by less bulky but equally lipophilic trichloromethyl groups (with the added advantage of conferring a pKa-lowering inductive effect).

Given that acid-association/-dissociation reactions occur at least to some extent within membranes, it may be argued that the proton shuttle cycle takes place entirely within the mitochondrial inner membrane. As such, the pattern of speciation derived from the membrane pKa value might be considered a better predictor of activity than that derived from the aqueous pKa, where membrane pKa is equal to aqueous pKa plus the difference between the log P_{membrane-water} values of the neutral species and of the ionized species [21A]. However, for several reasons, substitution of calculated membrane pKa for calculated aqueous pKa is unlikely to significantly enhance predictive power. First, the magnitude of the pKa shift can be expected to be relatively small and constant across all compounds of interest in this study. This is because ionized species generally partition more strongly into a membrane than into octanol (i.e., the log P_{membrane-water} difference is considerably smaller than the log P_{octanol-water} difference)[21A] and because all compounds of interest are characterized by good charge delocalization over a phenolic ring structure and thus by a minimized log P_{octanol-water} difference. While a small shift in pKa can significantly affect the speciation of compounds with a pKa close to cellular pH, it is of little consequence to the calculation of activity of powerful proton shuttles, as these are predicted to be characterized by much lower or higher pKa. More importantly, a nearly equal upward shift across compounds will not affect calculations of relative activity, as the model is designed to output. It should also be considered that the transformation of aqueous pKa into membrane pKa necessarily introduces additional error, compounded by the fact that log P_{membrane-water} values currently cannot be calculated from two-dimensional structure with the same reliability as log P_{octanol-water}. Along this line, the use of calculated membrane pKa as a predictor may be invalidated by failure to account for the difference in partitioning behavior between the membrane's hydrophilic domains and its hydrophobic core, or for the possibility that the electrical potential across the membrane polarizes the intramembrane distribution of the ionized species. These notions may be tested in future iterations of the model.

An obvious but unavoidable limitation of the present model is that it cannot predict potentiation of uncoupling activity mediated by interaction with proteins of the inner membrane. While protein-assisted uncoupling appears to generally require similar physicochemical properties as proton shuttle uncoupling (i.e., compounds must exist in neutral and ionized forms at IMS and matrix pH, must be lipophilic, and must exhibit extensive charge delocalization), interaction with a protein partner can be assumed to impose additional constraints, both structural and physicochemical. Given the difficulty involved in identifying the ideal physicochemical properties for uncoupling when all uncoupling mechanisms are confounded (for example see: [33A-36A]), it can be proposed that the constraints for interaction with a protein partner are somewhat at odds with the constraints for proton shuttle activity, and therefore that protein-assisted uncouplers must trade off proton shuttle activity for quality of interaction with their partner. It follows from this that proton shuttle activity will vary from one protein-assisted uncoupler to another independently of overall uncoupling activity. Case in point, while the present model necessarily underestimates the uncoupling activity of highly potent reference compounds such as SF-6847, FCCP, and CCCP as their activity is mediated in large part by protein interactions, the model predicts the two arylhydrazone uncouplers to be far better proton shuttles (CCCP>FCCP) than SF-6847. Along similar lines, the reported complexing of the neutral and ionized forms of certain uncouplers such as pentachlorophenol [37A], which clearly does not serve to increase the lipophilicity of an insufficiently lipophilic compound as does the dimerization of 2,4-dinitrophenol described in Section 3.6.3, can be proposed as necessary for interaction with a protein partner. Finally, it is proposed that any uncoupler that does not conform to the present model (e.g., platanetin [30A], usnic acid [31A]), regardless of potency, must be suspected of being a protein-assisted uncoupler, as would be the case if it exhibited sensitivity to inhibitors [38A], or more subtle signs such as a shallow dose-response relationship [10A, 12A] or sensitivity to minor structural modifications that do not affect physicochemical properties [18A].

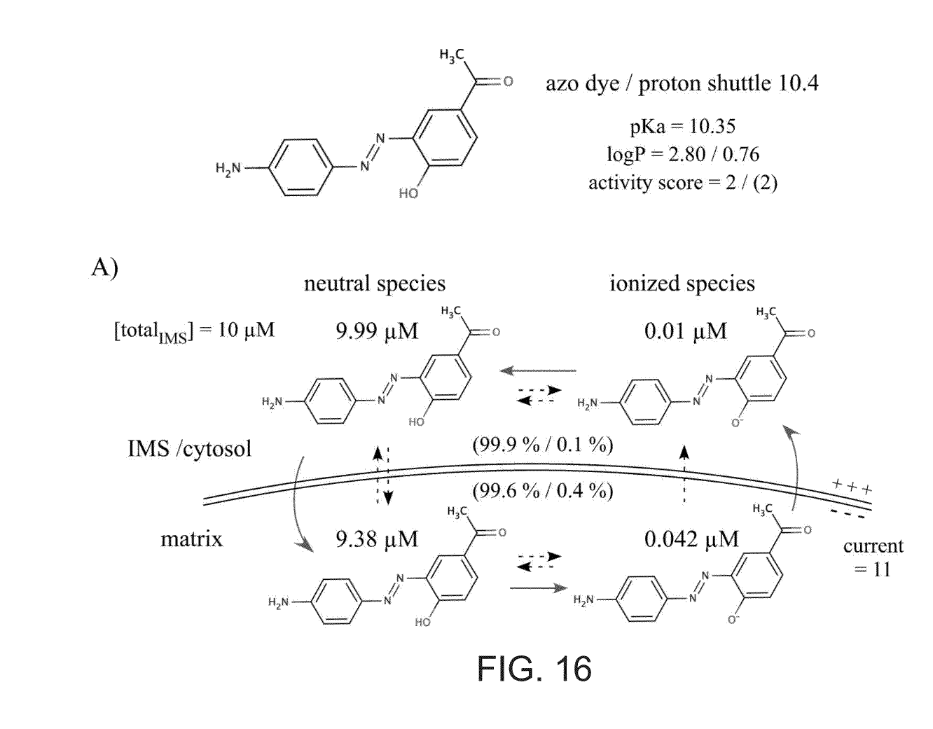

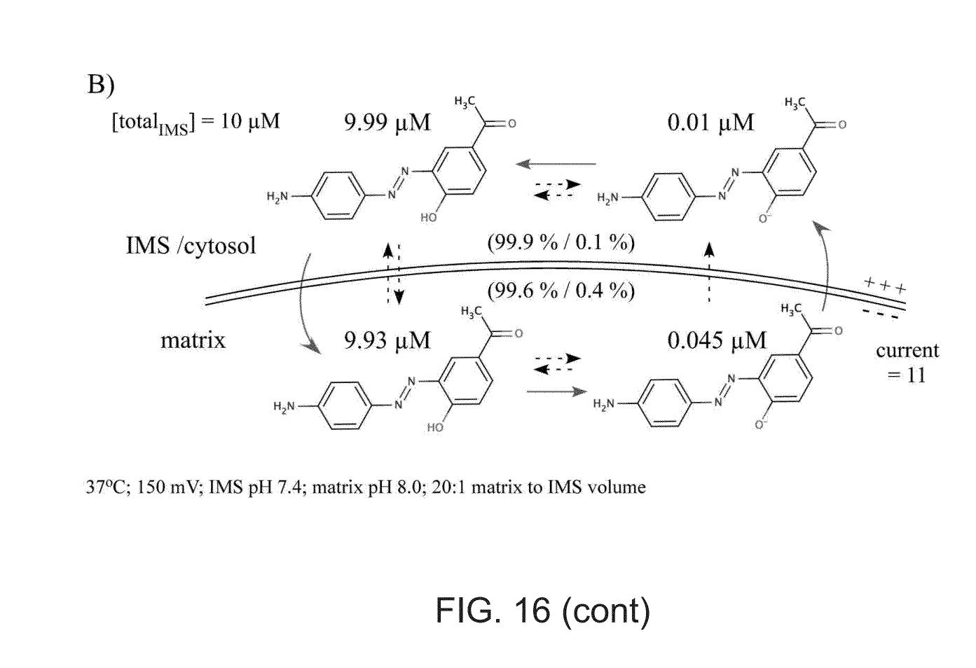

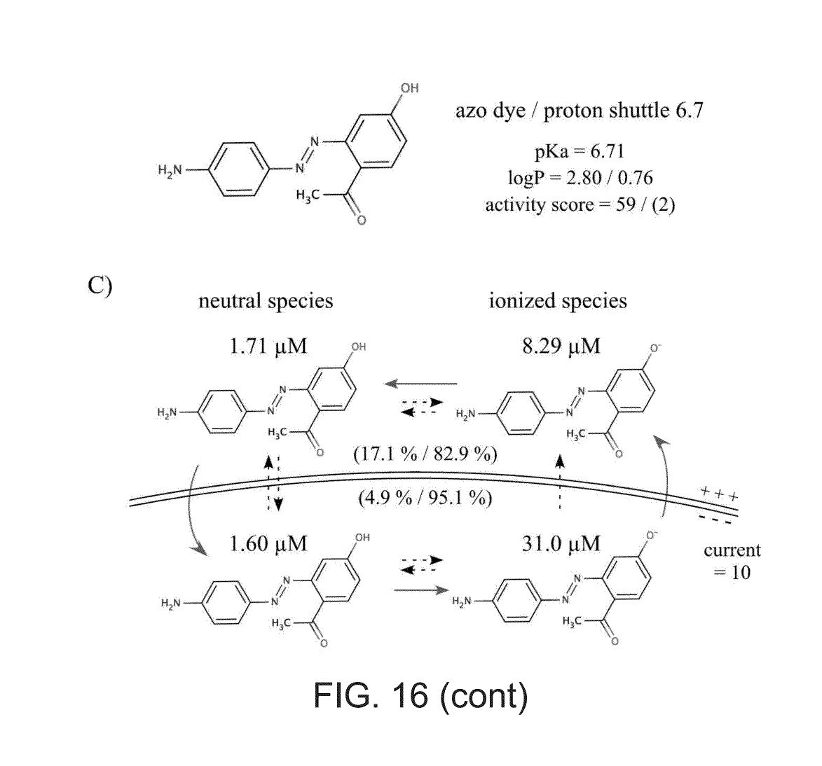

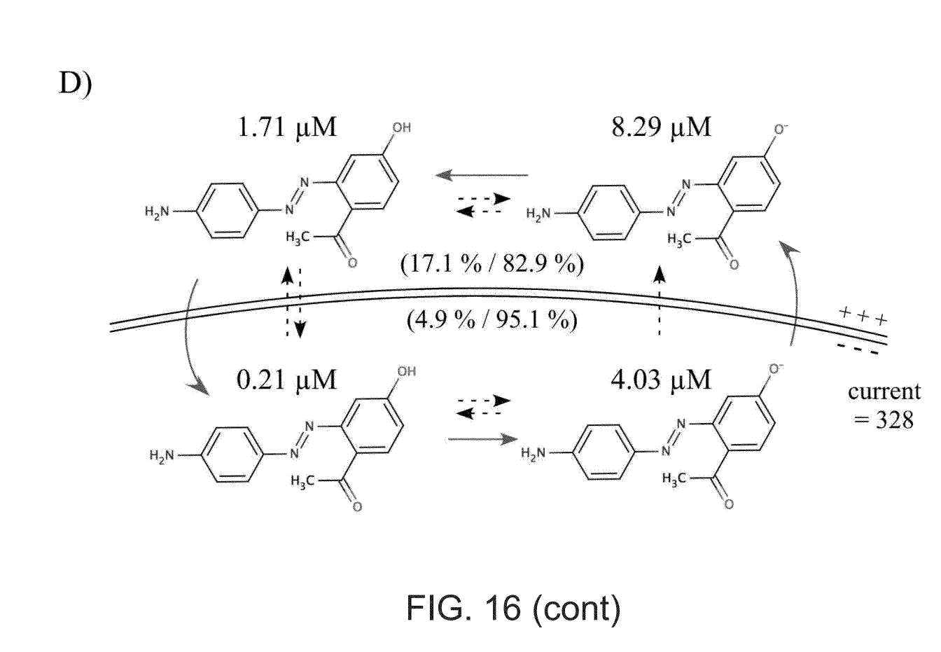

Further validation of the present work may be achieved by applying the model to the design and synthesis of novel uncouplers. These may simply be compounds optimized for high activity, such as examples presented in FIGS. 10 and 11, as well as in Supplemental Data Table 2, or they may be compounds designed to more specifically address aspects of the theoretical framework or the conclusions of the model. For example, the notion of a resistance-lowering effect of asymmetric distribution may be addressed by testing compounds designed such that their predicted activity is largely dependent on this effect. Similarly, multi-protic compounds can be designed such that their predicted activity is dependent on the shuttling of two protons per cycle iteration. It may also be possible to address the basic notions of distribution by designing proton shuttles from structures that exhibit halochromic properties (i.e., chromophores whose neutral and ionized forms exhibit different absorbance maxima, as in pH indicator dyes) for use with a microspectroscopic approach capable of resolving absorbance in subcellular compartments; from absorbances measured in the cytosol (representative of the mitochondrial IMS compartment) and in mitochondria (representative of the mitochondrial matrix compartment), IMS to matrix ratios of the concentrations of the neutral form and of the ionized forms could then directly be assessed and compared to predicted ratios calculated with and without the proposed resistance-lowering effect of distribution (FIG. 16). Anthraquinones and azobenzenes may be appropriate for this purpose.

It is likely that the theoretical framework developed can be useful for predicting proton shuttle activity of xenobiotics not only in higher animals but also in a wide variety of organisms for a variety of industrial purposes related to energy starvation. With little or no modification, the model may be suitable for predicting activity in insect and fungal mitochondria for the purposes of identifying compounds with pesticidal or anti-fungal activities. By specifying parameters of transmembrane electrical potential, transmembrane pH gradient (and corresponding limits for compound pKa), and compartment volume ratio that reflect chloroplastic conditions (e.g., electrical gradient^{˜}25 mV; stroma pH^{˜}8; thylakoid space pH^{˜}5; large stroma to lumen volume ratio), the model may be adapted to the identification of compounds with herbicidal activity. Similarly, it may be adapted to the identification of anti-bacterial or bacteriostatic compounds by specifying parameter values representative of the intra- and extracellular conditions of neutrophilic, acidophilic or even alkalophilic bacteria. It should be appreciated, however, that activity under one set of conditions does not guarantee activity under another.

Of the potential therapeutic applications of uncouplers, decreasing metabolic efficiency for the purpose of weight control is perhaps the most obvious. Indeed, uncouplers have been of interest for the treatment of obesity since the 1930's when dinitrophenols were shown to be clinically efficacious at decreasing metabolic efficiency to induce negative energy balance [39A]. While these original products were removed from market due to concerns over the formation of cataracts [40A], this area of research is still actively pursued today [41A, 42A], with emphasis on improving safety [10A]. However, such an application can only be expected to succeed if uncoupling activity is chronically sustained, which in turn requires that activity be tightly controlled so as not to induce energy starvation and complications related to increased reliance on anaerobic metabolism (i.e., lactic acidosis). This is particularly challenging given the typically narrow range of concentration over which uncouplers exhibit dose-response [10A].

A more promising therapeutic application is the indirect stimulation of the AMPK signaling pathway through the use of short-acting uncouplers. AMPK is a cytoprotective monitor of energy homeostasis: in response to an overwork-induced elevation in the concentration of AMP and/or a decrease of ATP availability, AMPK triggers effects, both acute and transcriptional, for restoring and protecting energy homeostasis, including the inhibition of non-essential energy-consuming processes, the stimulation of substrate uptake, and the up regulation of the capacity for substrate uptake and oxidation [43A]; AMPK is central to mitochondrial biogenesis and the development of fatigue-resistance through exercise training [44A]. An effective method to activate AMPK for therapeutic purposes is to induce a perturbation of energy homeostasis through the transient disruption of mitochondrial energy transduction. Indeed, the highly successful anti-hyperglycemic drug metformin induces AMPK-mediated insulin-like inhibition of hepatic glucose output as a result of its inhibitory effect on complex I of the electron transport chain [45A, 46A], and similar drugs that inhibit oxidative phosphorylation at other sites are currently in advanced stages of development [47A]. Uncouplers have also been shown to effectively activate AMPK and induce effects of therapeutic relevance [48A]. In this regard, however, uncouplers may have intrinsic advantages over compounds such as metformin. To this point, it has been proposed that uncouplers are better suited to the activation of AMPK in tissues with important oxidative capacity such as skeletal muscle, since, unlike inhibitors of oxidative phosphorylation, they can perturb energy homeostasis without completely encroaching on respiratory capacity, thereby inducing AMPK activation without the need for compromised ATP availability [12A]. Key to the successful use of uncouplers as replacements to metformin and metformin-like drugs is that activation of AMPK is rapid and is sustained long after energy homeostasis is restored [16A], and perturbations of energy homeostasis therefore need not be sustained in order to indirectly trigger AMPK-mediated effects of therapeutic relevance. Thus, safety can be maximized by favoring short-lived, easily-metabolized compounds, unlikely to cause complications related to sustained metabolic stress or related to bioaccumulation. Proton shuttles are ideally suited for this since their unconstrained structure-activity relationship allows for great flexibility in the design of compounds optimized for both high activity and low toxicity. It should be noted that built-in susceptibility to degradation need not imply susceptibility to first-pass metabolism and low bioavailability since it can be combined with pro-drug strategies such as acetylation of vulnerable hydroxyl groups to increase the amount of proton shuttle reaching target tissues.

An objective of the present work was to support the notion that uncoupling activity is not dependent on the presence of cyano, nitro, and other notoriously difficult-to-metabolize groups that are associated with the best-known reference uncouplers. Indeed, other less problematic electron-withdrawing groups can be used in their stead to exert a pKa-lowering effect. Similarly, alkyl and alkene groups can be used rather than halogen atom substituents to impart an adequate level of lipophilicity. In this vein, focus throughout this work has been on compounds composed exclusively of C, H, and O, working under the assumption that such compounds are generally more easily metabolized than nitrogenous or halogenated counterparts. A conclusion drawn from the present model is that such compounds can be optimized so as to exhibit an U₅₀ on the order of 1 μM (and perhaps lower for highly optimized di-protic compounds) and to push the known limits of the proton shuttle mechanism. The design of easily-metabolized uncouplers is of relevance not only to therapeutic applications but also to industrial applications. Indeed, it is environmentally sound to design pesticides or wood preservatives that have low potential for bioaccumulation. Moreover, compounds with particularly low oral bioavailability may be of interest as food preservatives or anticaries and antiplaque agents. Along these lines, low bioavailability may be combined with properties that confer activity towards acidophilic bacteria but not neutrophilic bacteria so as to selectively target stomach Helicobacter pylori.

From this, it is proposed that the prediction of uncoupling activity can be further refined by including considerations of activity duration based on predicted ease or difficulty of absorption and metabolism. Indeed, the integral of the magnitude of uncoupling activity over time may be a better predictor of the degree of perturbation of energy homeostasis and of metabolic stress induced by a given compound than peak activity alone. Gross differences in duration of activity may be especially relevant to the evaluation of the potential for toxicity related to increased cellular reliance on anaerobic metabolism (i.e., acidosis). Even among compounds unlikely to bioaccumulate, such as those considered in the present work, subtle differences in susceptibility to transformation and deactivation by processes such as methylation, sulfanation, or glucuronidation that decrease lipophilicity or that target hydroxyl groups, may account for significant differences in therapeutic potential. In combination with a better understanding of the optimal pattern of perturbation of energy homeostasis (magnitude, duration, and frequency) for maximizing AMPK-mediated effects, such an approach may facilitate the identification of lead compounds for therapeutic applications.

Referring to FIG. 1: General Mechanism of Uncoupling by Lipophilic Weak Acids. Activity requires at a minimum that a compound exist in both neutral and ionized forms in both the mitochondrial inter-membrane space (IMS; cytosolic pH) and the mitochondrial matrix (approximately 0.5 pH units more basic), and that both forms be appreciably membrane-permeable. Such a compound must therefore be characterized by a pKa within approximately 4 units of mitochondrial pH (assuming a mono-protic acid), small size and moderate to high lipophilicity (viz. An octanol-water partition coefficient>100), and extensive delocalization of its charge such that the decrease in permeability incurred upon ionization is limited to approximately two orders of magnitude. As a compound meeting these conditions permeates the mitochondrion, it seeks to equilibrate across the inner membrane, but equilibrium cannot be achieved. The neutral species diffuses into the matrix where it dissociates according to a pattern more favorable to the ionized species than in the IMS (A). However, the ionized species is prevented from diffusing into the matrix by the large electrical potential across the membrane (viz. approximately 150 mV, negative inside); this potential also drives out of the matrix the ionized species newly-formed from the dissociation of the neutral species (B). The consequence of this asymmetry and of the constraints imposed by patterns of speciation is that the neutral species is perpetually subjected to a chemical diffusion gradient into the matrix, while the ionized species is perpetually subjected to an electrical diffusion (i.e., migration) gradient (potentially combined with a chemical diffusion gradient) out (C). The result is a cycle consisting of the diffusion of one molecule of the neutral species into the matrix coupled by mass action to the diffusion of one molecule of the ionized species out, with a proton carried into the matrix with each iteration (D). This dissipates the potential energy across the inner membrane that is necessary to the resynthesis of ATP; dissipation is precisely countered by an increase in the rate of proton pumping out of the matrix by the electron transport chain, in so far as spare respiratory capacity is available (not illustrated). An uncoupler whose activity is entirely attributable to this mechanism is termed a proton shuttle. Renewed interest in this mechanism lies in the therapeutic application of xenobiotic uncouplers to the induction of transient metabolic stress for the indirect activation of AMPK signaling; the mechanism is illustrated here with the plant metabolite 4′-hydroxychalcone (pKa^{˜}7.9; log P_{octanol-water}^{˜}3.6), the parent of a family of weak uncouplers that induce remarkable insulin-like activities in liver and skeletal muscle cells [12A].

Referring to FIG. 2: Thermodynamic Considerations of Uncoupling by Proton Shuttles. A) The sum of the Gibbs free energies (A/G) for the diffusion of a mole of the neutral species of a proton shuttle into the matrix (or matrix side of the system), the dissociation of a mole the neutral species in the matrix, the diffusion of a mole of the ionized species into the IMS (or IMS side), and the association of a mole of the ionized species in the IMS must equal the ΔG for the translocation of a mole of protons across the inner membrane down its concentration and electrical gradients. As the dissociation and association steps are assumed to be fast and to cancel out, the cycle reduces to two diffusional steps. B) Under the specified conditions, the ΔG for the translocation of protons, and therefore the sum of the ΔG's for the two diffusional steps, is calculated to be −18,034 J./mole (negative sign denoting an exergonic process). Given that the electrical contribution to the ΔG for the diffusion of the ionized species is fixed at −14,473 J./mole, in accordance with a valence of −1, then concentrations of the neutral and the ionized species are constrained as follows: 1) the ΔG for the diffusion of the neutral species into the matrix must be negative; 2) the chemical contribution to the ΔG for the diffusion of the ionized species out of the matrix must be >−3,561 J./mole; 3) the sum of the ΔG for the diffusion of the neutral species in and of the chemical contribution to the ΔG for the diffusion of the ionized species out must equal −3,561 J./mole; 4) patterns of speciation dictated by pKa and compartment pH are inviolable. C) Applying an electrical analogy, each diffusional step is considered as having a driving potential corresponding to its respective ΔG, and an intrinsic resistance to diffusion; respective diffusional flux or “current” is estimated by dividing potential by resistance. D) Since the two diffusional steps are coupled by mass action, they have equal flux under steady-state conditions. From this, it is postulated that while still respecting the constraints outlined above, concentrations of the neutral and ionized species under steady-state conditions are such that the resulting ΔG's for the diffusional steps are proportional to the respective intrinsic resistances, and the quotient of ΔG and resistance is therefore equal for both species. E) From this, the system can be considered a closed circuit with a driving potential of 18,034 J./mole and a total resistance equal to the sum of two resistances in series. Dividing potential by total resistance yields current, used in its proper electrical sense, at any point within the circuit. Current multiplied by potential yields power or rate of energy dissipation, the ultimate expression of uncoupler activity at a given uncoupler concentration. Activity of a proton shuttle is therefore dictated by the intrinsic resistances to diffusion of its neutral and ionized species.

Referring to FIG. 3: Proposed Resistance-Lowering Effect of Asymmetric Molecular Distribution. Conditions of asymmetric distribution are proposed to effectively decrease the resistance to diffusion of either the neutral or ionized species of a proton shuttle. This notion is developed using a simplified system. In (A), the single neutral molecule on the IMS side and the single ionized molecule on the matrix side bridge the diffusional distance (which can be taken to be the width of the membrane) at a terminal velocity determined by their respective driving potential and resistance to diffusion. These then respectively undergo dissociation and association, whereupon the cycle begins anew. Although terminal velocities may differ, the dissociation/association steps occur simultaneously and require that both molecules have completed their respective diffusion process. Resistance of the ionized species is 9-fold greater than that of the neutral species. Driving potential is considered independent of molecular distribution. If driving potential is shared equally between species, then diffusion of the ionized species is rate-limiting to the cycle (left). However, if potential is attributed such that both species travel the same distance per unit time, then the cycle turns at its maximal rate (right). In (B), there are three ionized molecules for one neutral molecule. As before, one neutral molecule is exchanged for one ionized molecule with each iteration of the cycle, and the resistance of the ionized species is 9-fold that of the neutral species. If all three ionized molecules can bridge the diffusional distance simultaneously but only one can undergo association in any given iteration, then, under steady-state conditions, ionized molecules assume equidistant spacing corresponding to one third of the diffusional distance. Consequently, the time needed for any one molecule to bridge the distance from the start of a cycle, and hence the resistance to diffusion, is effectively decreased by a factor of three. As before, maximum cycling rate is achieved when both species travel the same distance per unit time. Maximal rate is higher for (B) than for (A) as the same potential is divided by a total resistance of 1+3, rather than 1+9. In (C) and (D), these notions are applied to 4′-hydroxychalcone: (C) summarizes steady-state conditions calculated only from pKa and the ratio of the predicted resistances of the two species, whereas (D) factors in the resistance-lowering effect of an excess of matrix ionized molecules relative to the number of IMS neutral molecules. Note that the 6-fold resistance-lowering effect is smaller than predicted from the 20-fold molecular excess calculated in (C); unlike in the simplified system, potential and distribution are indissociable and a decrease in resistance commands the rebalancing of distribution. Calculations assume the following conditions: 37° C.; IMS pH 7.4; matrix pH 8.0; 150 mV transmembrane potential; matrix to IMS volume ratio 20:1; total (neutral+ionized) concentration of proton shuttle in the IMS 100

Referring to FIG. 4: Contributions of Partitioning Behavior and of Molecular Size and Shape to the Estimation of Permeability/Resistance to Diffusion. Membrane permeability, or its inverse, resistance to diffusion, is a prime determinant of proton shuttle activity. Permeability behavior is here estimated from the octanol-water partition coefficient (P_{octanol-water}), a measure of lipophilicity, calculated independently for the neutral and the ionized species, and, to a lesser extent, from an index of molecular size and shape based on minimal projection area. Optimal lipophilicity for the diffusion of proton shuttles across the inner membrane was set to log P_{octanol-water} 3.2, a value conferring a best fit in Section 4.1 and likely representing a compromise between the optimum for diffusion through the membrane's hydrophilic boundary layers and outer surfaces and the optimum for diffusion through its hydrophobic core. Deviation from this optimum, either positive or negative, was taken to linearly decrease permeability as the viscosity of interaction between the diffusing compound and the molecules of the medium through which it is diffusing increases in both cases; the function used to relate values of log P_{octanol-water} to relative permeability is plotted in (A). Based on an assumption of anisotropic diffusion, it is proposed that the favored spatial orientation of a proton shuttle as it is driven through the membrane is such that the area of the surface perpendicular to the direction of travel is minimized. This minimized frontal area was assessed as the smallest area that can be projected from the three-dimensional rendering of a compound in its lowest-energy conformation and based on van der Waals atomic radii. Relative permeability was taken to decrease with the square of this area and linearly with compound length measured perpendicularly to this area, or z-length, in accordance with notions of viscous drag (B); permeability was expressed relative to phenol, the parent structure of all compounds considered in the present study. Minimal projection area and z-length are illustrated for 4′-hydroxychalcone (C). At the request of the author, calculations of these parameters have been implemented in the ChemAxon Marvin chemoinformatics suite; a screenshot taken from version 5.3 is shown (D). The product of the square of minimal projection area and of z-length was plotted against molecular mass for the 283 non-ionic compounds considered in Sections 4.1 and 4.2 (E). From this, it can be appreciated that this index better resolves asymmetric compounds than mass, and effectively captures the range of molecular shape possible at any given mass. The depicted relationship also suggests that geometric considerations rapidly become prohibitive to efficient diffusion beyond 300 Da.

Referring to FIG. 5: Plot of the Interaction of pKa and Lipophilicity. Proton shuttle activity is proposed to be determined by a multi-level interaction between acid-dissociation behavior and permeability behavior. This interaction can be appreciated from a surface plot of the activity predicted to result from various combinations of pKa and neutral species log P_{octanol-water} for a mono-protic acid under conditions where geometric considerations and the log P_{octanol-water} value of the ionized species are controlled. This exercise also allows for appreciation of the range within which activity is predicted to vary. Activity was calculated under the following conditions and is expressed in multiples of 10⁶ arbitrary units (a.u.) of rate of energy dissipation: 37° C.; IMS pH 7.4; matrix pH 8.0; transmembrane electrical potential 150 mV; matrix to IMS volume ratio 20:1; log P_{octanol-water} optimum for diffusion 3.2; pKa range compatible with activity 4.0 to 11.4. The magnitude of decrease in log P_{octanol-water} upon ionization was fixed at 2.1, as typical of phenolic compounds. The product of minimized frontal area and z-length was set to the minimum achievable at any given value of log P_{octanol-water} for compounds composed exclusively of C, H, and O, thereby reflecting the increase in bulk required to achieve high lipophilicity. Two distinct activity spaces emerge: a primary space describing compounds whose neutral species exhibits less resistance to diffusion than its ionized species, and a secondary space describing compounds whose neutral species is excessively lipophilic and exhibits more resistance to diffusion than its ionized species. On the basis of Section 4.1, the activity scale can be calibrated as follows: 10×10⁶ a.u. (indicated by a dotted line) is the threshold for measurable uncoupling activity; 50×10⁶ a.u. (first solid isobar) is a rough threshold for activity of physiological relevance; 100×10⁶ a.u. (second solid isobar) corresponds to an U₅₀ on the order of 10 μM. From this, the peak of the primary activity space can be proposed to correspond to an U₅₀ on the order of 1 μM if a 1:1 relationship between proton shuttle concentration and activity is assumed. This peak is slightly higher if compounds capable of exceptionally extensive charge delocalization are considered, but halogenation is not predicted to significantly improve maximal activity.

Referring to FIG. 6: Thermodynamic Considerations for Multi-Protic Proton Shuttles. It is postulated that the activity of a multi-protic proton shuttle is mediated by the single most efficient combination of the neutral species and one of the ionized species that exist at mitochondrial pH. The model predicts that under certain circumstances, this most efficient combination can be made up of a di-ionized species rather than a mono-ionized species. When this occurs, two protons are translocated per cycle. The ΔG per mole uncoupler driving such a cycle is therefore twice the ΔG for the translocation of a mole of protons, and the concentrations of the neutral and di-ionized species must be such that the sum of the ΔG's for their diffusion is equal to this value. Under the specified conditions, such a cycle will be driven by −36,068 J./mole uncoupler; given that the electrical contribution to the ΔG for the diffusion of the di-ionized species is fixed at −28,946 J./mole, in accordance with a valence of −2, then the concentrations of the neutral and of the di-ionized species must be such that the sum of the ΔG for the diffusion of the neutral species and of the chemical contribution to the ΔG for the diffusion of the di-ionized species equals −7,122 J./mole, while respecting patterns of speciation. FIG. 12 summarizes the calculation of steady-state conditions for the three possible circuits of an example di-protic compound.

Referring to FIG. 7: Special Cases of the Proton Shuttle Mechanism: Cations and Weak Lipophilic Bases. The theoretical framework can reconcile the activity of cationic compounds (A) and of basic compounds (B). In these special cases, the proton shuttle cycle is best described in terms of protonated and deprotonated species. In the first case (illustrated by the naturally-occurring chromenylium ion structure, hydroxy-substituted at position 3), the compound is positively charged rather than neutral when in protonated form, whereas in deprotonated form, its positive and negative charges cancel out and the compound exhibits no net charge instead of a negative charge. Accordingly, the transmembrane electrical potential acts on the protonated species rather than on the deprotonated species, driving diffusion of the charged species into the matrix rather than out; the deprotonated species diffuses out of the matrix down a chemical gradient. In the second case (illustrated by the naturally-occurring isoquinoline structure), the protonated species corresponds to the positively-charged ionized species, whereas the deprotonated species corresponds to the neutral species. As above, it is the protonated species that is subject to an electrical gradient, and this gradient drives diffusion into the matrix rather than out; the neutral species diffuses out of the matrix down a concentration gradient. This cycle is therefore reversed relative to that of an acidic proton shuttle. The model can be adapted to predict the activity of cationic and basic proton shuttles by taking into account their special electrical behavior; FIGS. 13 and 14 summarize the calculation of steady-state conditions and activity for two examples of cationic and basic compounds, respectively.

Referring to FIG. 8: Special Case of the Proton Shuttle Mechanism: Compounds Capable of Overcoming Insufficient Lipophilicity Through Oligomerization. The theoretical framework can reconcile the activity of compounds that shuttle protons in oligomeric form, as exemplified by the reference uncoupler 2,4-dinitrophenol. In monomeric form, lipophilicity of this compound (log P_{octanol-water} 1.55) is incompatible with the efficient shuttling of protons (A). However, 2,4-dinitrophenol undergoes spontaneous dimerization, presumably through strong hydrogen-bonding between a nitro and a hydroxyl group, resulting in an approximate doubling of log P_{octanol-water} and therefore in near-optimal lipophilicity. The advantage associated with this increase in lipophilicity outweighs the accompanying increase in molecular bulk, and proton shuttle activity is therefore predicted to be significantly greater in dimeric than in monomeric form. Note that two non-equivalent dimers are possible: both are predicted to be characterized by near-optimal lipophilicity and to incur a comparable increase in molecular bulk, but only one is expected to retain the near-optimal pKa of the monomer; one isoform is predicted to shuttle protons with ten-fold greater activity than the monomer (B), whereas the other is predicted to do so with only two-fold greater activity (not shown). Given knowledge of the mitochondrial distribution of the monomeric and various oligomeric forms of the compound, the model can be adapted to predict overall proton shuttle activity by summating activities predicted individually for each form, weighted according to respective effective concentration. Note that nitro groups have been drawn so as to indicate equivalency between oxygen atoms.

Referring to FIG. 9: Relationship Between Predicted and Measured Activity for a 48-Compound Testset. Validity of the theoretical framework and its proposed mathematical implementation was assessed by comparing predicted proton shuttle activity against experimentally-measured uncoupling. This was performed for a large test set of naturally-occurring phenolic compounds that span a wide range of structures and physicochemical parameter values. The most active of these exhibit an U₅₀ on the order of 10 μM and can therefore be counted among the most powerful proton shuttles known. Compound descriptors and measured uncoupling activity, reported elsewhere [12A] are summarized in Supplemental Data Table 1. Uncoupling activity is expressed as the increase in state 4 respiration of isolated rat liver mitochondria treated with 100 μM of test compound, relative to ADP-stimulated respiration set to 100%. This standardized test concentration falls within the linear portion of most test compounds' dose-response relationship. Reported activity represents the average of measurements performed over 2 to 3 independent mitochondrial preparations; error bars show SEM. Predicted proton shuttle activity is based on calculated rather than experimentally-determined physicochemical data and is expressed as multiples of 10⁶ arbitrary units of rate of energy dissipation. A satisfactory fit between predicted and measured activity is indicated by a Spearman rank order correlation coefficient of 0.90. The nine compounds with significant uncoupling activity were successfully resolved from the rest of the test set despite pKa₍₁₎ values ranging from 4.5 to 7.9 and a variable number of ionization sites.

Referring to FIG. 10: Naturally-Occurring Chemical Templates Conducive to Proton Shuttle Activity and Examples of Derivatives Optimized for High Activity. Ten hydroxy-substituted structures (left) were identified as core elements of naturally-occurring proton shuttles and as potential templates for the design of optimized synthetic derivatives. These structures are characterized by pKa equal to or below mitochondrial pH and by a propensity for extensive charge delocalization. Optimized derivatives can be designed by the addition of electron-withdrawing substituents at positions denoted by an asterisk to further decrease pKa, and the addition of lipophilic substituents, preferably coplanar with the core structure and positioned so as to have the least impact on the structure's frontal area, to increase log P_{octanol-water} to approximately 3.2. For each template, a derivative designed for maximal activity but composed exclusively of C, H, and O is proposed (right): formyl or acetyl substituents are used to optimize pKa, whereas planar lipophilic substituents such as methyl, ethenyl, propen-1-yl, and 1-methylpropen-1-yl are used to optimize lipophilicity. These examples are predicted to exhibit an U₅₀ on the order of 1 μM. At the risk of resulting in more difficult-to-metabolize derivatives, activity can be slightly increased on the basis of reduced molecular bulk by using halogenated substituents rather than alkyls and alkenes to achieve optimal lipophilicity. Predicted activity (expressed in multiples of 10⁶ arbitrary units of rate of energy dissipation) and calculated values of pKa and log P_{octanol-water} (neutral/ionized forms) are indicated beside each example. Additional examples are listed in Supplemental Data Table 2. The proposed hydroxychromenylium derivatives may be unstable and impossible to synthesize.

Referring to FIG. 11: Optimized Di-Protic Derivatives with Greater Predicted Activity than their Mono-Protic Counterparts. The model allows for the possibility that a di-protic compound highly-optimized in terms of lipophilicity and geometry can be more active than any of its mono-protic counterparts if its di-ionized species benefits from a near-maximal resistance-lowering effect of distribution. For this, pKa₍₁₎ and pKa₍₂₎ must be jointly minimized without precluding the existence of the neutral species at matrix pH. Such exceptional derivatives can be designed from five of the ten templates of FIG. 10, and proposed examples are illustrated. These are predicted to exhibit an U₅₀ on the order of 500 nM to 1 μM and to be more powerful than any known proton shuttles. As before, derivatives predicted to be slightly more active on the basis of geometric considerations are possible if design is not restricted to C, H, and O atoms. Predicted activity (expressed in multiples of 10⁶ arbitrary units of rate of energy dissipation) and calculated values of pKa₍₁₎, pKa₍₂₎, and log P_{octanol-water} (neutral/ionized forms) are indicated for each example. The proposed dihydroxychromenylium derivative may be unstable.

Referring to FIG. 12: Evaluation of the Potential Circuits of a Di-Protic Compound. The activity of a multi-protic proton shuttle can, in principle, be mediated by its neutral species in combination with any of its ionized species. It is postulated, however, that activity is mediated exclusively by the single most efficient combination, or the “circuit” that can dissipate energy at the greatest rate. In order to identify this most efficient circuit, steady-state concentrations, the resistance-lowering effect of distribution, and activity must be calculated independently for each of the possible circuits, as for the circuit of a mono-protic compound. In the example of the di-protic 2,4′-dihydroxychalcone, three circuits are possible: two involving the neutral species and one of the mono-ionized species (circuits 1 and 2), and a third involving the neutral species and the di-ionized species (circuit 3). Of these, circuit 1 is expected to be the one through which energy can be dissipated at the highest rate. Circuits 2 and 3 are less efficient because their respective ionized species are less lipophilic than that of circuit 1 (only slightly less so in the case circuit 2) and therefore have greater resistance to diffusion, and because their respective ionized species are less prevalent than that of circuit 1 and therefore benefit from a smaller resistance-lowering effect of distribution. It should be noted that in circuit 3, the ionized species carries two protons per cycle iteration, and neutral molecules are therefore exchanged for ionized molecules at a ratio of 2:1; the driving potential per mole of uncoupler of this circuit is twice that of circuits 1 and 2, and calculations of the resistance-lowering effect of distribution for this circuit take into account the unequal exchange ratio. These advantages offset in large part the decrease in lipophilicity of the di-ionized species relative to the mono-ionized species; given a more favorable distribution and a larger resistance-lowering effect, it would be possible for circuit 3 to be more efficient than circuits 1 or 2. Finally, it should be noted that the steady-state concentrations of all species must conform to the patterns of speciation dictated by acid-dissociation behavior, and the concentrations (IMS/matrix in μM) of ionized^{-1B} and ionized⁻² in circuit 1 are calculated as 2.5/4.5 and 0.9/6.0, respectively; the postulate of equal flux and maximal current dictates not only the steady-state concentrations of the species mediating proton shuttle uncoupling, but also those of non-contributing species.

Referring to FIG. 13: Adapting the Model to Predict the Activity of Cationic Compounds. The model can readily be adapted to the prediction of proton shuttle activity in cationic compounds by accounting for the gain of a positive charge on the protonated side and the corresponding net loss of a negative charge on the deprotonated side. Calculations of steady-state conditions and activity are summarized here for the compound 3-hydroxyflavylium (A), derived from the flavylium ion backbone of the anthocyanidin class of flavonoids. This example is predicted to be well suited to proton shuttle activity due to its pKa of approximately 5 and to the exceptional capacity for negative charge delocalization of the chromenylium ring structure (decrease in log P_{octanol-water} upon ionization of less than 1.4 units). Like their non-ionic counterparts, cationic proton shuttles can be mono-protic or multi-protic acids. Applying the rationale developed for non-ionic compounds, assessment of the activity of a multi-protic cationic compound requires that all possible circuits be considered, including those that involve a species ionized at multiple sites and through which more than one proton can be shuttled per cycle iteration. Interestingly, in the case of cationic compounds, circuits involving the protonated species and a multi-ionized species are characterized by an electrical diffusion gradient acting on both species; the positively-charged species is driven into the matrix while the (net) negatively-charged species is driven out. Such a circuit, composed of the protonated species and a di-ionized species, is illustrated for the compound 3,5-dihydroxyflavylium (B). Along this same line, it is an intriguing possibility that the involvement of the fully protonated species is not essential and that a circuit might be composed of two deprotonated species: an electrically neutral mono-deprotonated species and a multi-deprotonated species with a net negative charge (not shown).

Referring to FIG. 14: Adapting the Model to Predict the Activity of Basic Compounds. While lipophilic weak base uncouplers occur less frequently than lipophilic weak acid uncouplers, there is no reason to expect that they should be less conducive to activity than their acidic counterparts, given equivalent physicochemical properties. The relationships governing resistance to diffusion (Section 3.3) should apply equally to bases as to acids. Moreover, it can be expected that the activity of bases is modulated by basic pKa in accordance with a mathematical relationship similar to that developed for acids (Section 3.2), only reversed such that activity increases with increasing rather than decreasing basic pKa (up to the arbitrarily-fixed limit of 11.4). Therefore, the model can be adapted to the prediction of proton shuttle activity in basic xenobiotics simply by reversing certain functions. Calculations of steady-state conditions and activity are summarized here for an isoquinoline derivative with near-optimal lipophilicity (A). From a design perspective, it is noteworthy that the ring system of this compound is predicted to exhibit very extensive charge delocalization (decrease in log P_{octanol-water} upon ionization of as little as 1 unit) and therefore appears to be well-suited to proton shuttle activity. However, the same resonance considerations responsible for this phenomenon also impart to this compound an undesirably low basic pKa. Interestingly, just as the model predicts a secondary activity space for acidic compounds (Section 3.2; FIG. 5), it predicts that low basic pKa is conducive to activity of a basic compound when such a compound exhibits greater permeability in ionized form than in neutral form (i.e., excessive lipophilicity in neutral form): activity is maximized when basic pKa approaches 4.0 and lipophilicity of the ionized rather than the neutral species approaches the specified log P_{octanol-water} optimum of 3.2. An example of a compound designed around these considerations is illustrated (B). The design of optimized basic proton shuttles is not considered further in the present work since basic templates conducive to activity are fewer than acidic templates, basic compounds are not amenable to substituent-induced increase in pKa, and equivalent or superior predicted activity can be achieved with non-nitrogenous compounds.

Referring to FIG. 15: Example of Proton Shuttle Exhibiting Cis-Trans Photoisomerization. The importance of compound shape to activity may be tested with proton shuttles designed around the azobenzene structure, or other such structure that exhibits photoisomerization properties. The trans-isomer of the illustrated compound is predicted to exhibit high proton shuttle activity (left). However, activity of the cis-isomer (right), kinked and non-planar, is predicted to be reduced by approximately one half relative to that of the trans-isomer on the basis of geometric considerations. Under UV illumination, the compound can be converted from the stable trans-isomer to the higher energy cis-isomer. Thermal back-relaxation occurs within seconds to minutes. Alternatively, cis- to trans-conversion can be prompted with blue light illumination. It may therefore be possible to modulate the activity of this compound in real time in isolated mitochondria by toggling back and forth between its two isomers.

Referring to FIG. 16: Halochromic Proton Shuttles Designed for the Direct Assessment of Molecular Distribution. The notions of molecular distribution on which the model is built may be directly tested using weak proton shuttles designed from the skeleton of a pH-sensitive chromophore in conjunction with a microspectroscopic approach. In this way, concentrations of proton shuttle in neutral and in ionized forms in the IMS and matrix compartments may be estimated by measuring absorbance at the chromophore's two maxima in the cytosol and in mitochondria. From this, the IMS to matrix ratios of neutral form and ionized form concentrations may be assessed and compared to predicted ratios, calculated with and without the contribution of the proposed resistance-lowering effect of distribution (Section 3.2). Since this approach assesses distribution rather than activity, proton shuttles can be designed for low activity so as to ensure that the membrane potential is not collapsed during the experiment. A series of such probes differing only in acid-dissociation behavior may be designed and tested. Multi-protic compounds may also be included. The predicted steady-state conditions of two proton shuttles derived from an azo-type pH indicator are illustrated. The two compounds are predicted to exhibit significantly different acid-dissociation behavior, but to be comparable in other respects. The compound on the left does not benefit from a resistance-lowering effect of distribution acting on its ionized species due to its overly high pKa of 10.4, and its activity is predicted to be negligible whether the resistance-lowering effect is ignored (A) or taken into account (B). In accordance with the postulate of equal fluxes and maximum current (Section 3.1), a large difference is expected between the IMS to matrix ratio of the neutral species concentrations (approximately 1:1) and the IMS to matrix ratio of the ionized species concentrations (approximately 1:4). In contrast, the compound on the right is expected to benefit from a significant resistance-lowering effect acting on its ionized species on the basis of its pKa of 6.7. If this effect is ignored (C), then the compound's activity and its IMS to matrix ratio of ionized species concentrations are calculated to be comparable to that of the compound on the left. However, if the resistance-lowering effect is taken into account (D), then the compound is expected to have measurable proton shuttle activity and its IMS to matrix ratio of ionized species concentrations is calculated to be approximately 2:1, or 8-fold higher than in (C) or than that of the compound on the left.

Referring to Tables 1A and 1B: Descriptors of the Most Active Compounds Tested and of Active Compounds Tentatively Identified Through in-silico Screening.

Notes to Table 1A and 1B: Complete 48 compound validation testset summarized in Supplemental Data Table 1. Predicted proton shuttle uncoupling activity reported in arbitrary units of rate of energy dissipation/mole uncoupler; 100×10⁶ a.u. corresponds approximately to an U₅₀ of 10 μM. Uncoupling activity measured in isolated rat mitochondria; methodology does not differentiate between proton shuttle activity and protein-assisted uncoupling activity. Some compounds of screening testset may not be naturally-occurring. Predictions of activity in flavylium cations are highly tentative as compounds may be unstable. Acid-dissociation constants and octanol-water partition coefficients calculated using ChemAxon Marvin 5.2. Minimal projection area and z-length perpendicular to minimal projection area calculated using ChemAxon Marvin 5.3. PubChem compound database, National Center for Biotechnology Information (http://pubchem.ncbi.nlm.nih.gov). ChemSpider compound database, Royal Society of Chemistry (http://www.chempider.com).

TABLE 1A
Compounds From Validation Testset Exhibiting Highest Uncoupling Activity.

| | | | | Calculated | | | Measured | Predicted | |
|---|---|---|---|---|---|---|---|---|---|
| | | | | logP | Minimal | z- | Uncoupling | Proton Shuttle | Report of |
| | | | Calculated | (neutral | Projection | length | (% uncoupling ± | Activity | Uncoupling |
| Compound name | CAS # | Chemical Class | pKa | Species) | Area (Å²) | (Å) | SEM @ 100 μM) | (×10⁶ a.u.) | Activity |
| frangulic acid | 518- | anthraquinone | 7.39; 9.44; | 3.82 | 33.90 | 13.21 | 78 ± 14 | 74 | |
| | 82-1 | | 10.23 | | | | | | |
| butein | 487- | chalconoid | 7.75; 8.77; | 3.33 | 30.57 | 15.32 | 71 ± 4 | 83 | ref. [25A] |
| | 52-5 | | 9.35; 12.46 | | | | | | |
| homobutein | 34000- | chalconoid | 7.77; 8.96; | 3.47 | 36.15 | 15.42 | 56 ± 5 | 56 | |
| | 39-0 | | 9.65 | | | | | | |
| 4′-hydroxychalcone | 2657- | chalconoid | 7.87 | 3.59 | 26.27 | 15.07 | 74 ± 14 | 81 | ref. [26A] |
| | 25-2 | | | | | | | | |
| isoliquiritigenin | 961- | chalconoid | 7.75; 8.78; | 3.63 | 28.74 | 15.54 | >100 | 77 | ref. [25A] |
| | 29-5 | | 9.36 | | | | | | |
| galangin | 548- | flavonoid: | 4.79; 6.85; | 2.76 | 35.84 | 13.51 | 95 ± 9 | 101 | ref. [27A] |
| | 83-4 | flavonol | 9.57 | | | | | | |
| biochanin A | 491- | flavonoid: | 6.61; 9.29 | 3.22 | 35.28 | 16.19 | >100 | 91 | |
| | 80-5 | (iso)flavone | | | | | | | |
| chrysin | 480- | flavonoid: | 6.64; 9.32 | 3.01 | 35.04 | 13.51 | >100 | 70 | ref. [23A] |
| | 40-0 | (iso)flavone | | | | | | | |
| genistein | 446- | flavonoid: | 6.61; 8.79; | 3.08 | 31.91 | 14.98 | 74 ± 4 | 92 | |
| | 72-0 | (iso)flavone | 9.46 | | | | | | |

TABLE 1B
Compounds From in-silico Screening Testset Predicted to Exhibit Significant Proton Shuttle Activity.

| | CAS #, | | | | | | | |
|---|---|---|---|---|---|---|---|---|
| | PubChem | | | Calculated | | | Predicted | |
| | CID, or | | | logP | Minimal | | Proton Shuttle | Report of |
| | Chemspider | | Calculated | (neutral | Projection | z-Length | Activity | Uncoupling |
| Compound Name | ID | Chemical Class | pKa | species) | Area (Å²) | (Å) | (×10⁶ a.u.) | Activity |
| 2-hydroxy-10-anthrone | 5449-65-0 | anthracene | 7.88 | 3.25 | 27.80 | 12.50 | 94 | |
| alizarin | 72-48-0 | anthraquinone | 7.55; 13.05 | 2.96 | 27.36 | 12.33 | 86 | ref. [29A] |
| averufanin | 28458-24-4 | anthraquinone | 6.49; 7.19; | 4.21 | 44.02 | 15.94 | 46 | |
| | | | 10.52; 11.26 | | | | | |
| 3,7-dimethyl anthraflavic | none | anthraquinone | 7.38; 7.99 | 3.34 | 32.44 | 13.72 | 104 | |
| acid | available | | | | | | | |
| 2-hydroxy | 605-32-3 | anthraquinone | 7.30 | 2.62 | 26.63 | 12.47 | 53 | |
| anthraquinone | | | | | | | | |
| purpurin | 81-54-9 | anthraquinone | 7.29; 11.65; | 3.31 | 31.10 | 12.21 | 144 | |
| | | | 14.71 | | | | | |
| questin | 3774-64-9 | anthraquinone | 7.03; 10.25 | 3.32 | 34.97 | 13.00 | 130 | |
| auronol | PubChem | benzofuran | 5.86 | 3.86 | 30.52 | 13.82 | 130 | |
| | 27562062 | | | | | | | |
| cardamonin | 76-22-2 | chalconoid | 7.20; 9.10 | 3.78 | 34.54 | 14.62 | 56 | |
| echinatin | 34221-41-5 | chalconoid | 7.83; 8.86 | 3.13 | 33.47 | 15.32 | 45 | ref. [26A] |
| eriodictyolchalcone | 14917-41-0 | chalconoid | 7.07; 8.62; | 3.67 | 34.38 | 15.49 | 60 | |
| | | | 9.19; 10.07; | | | | | |
| | | | 12.47 | | | | | |
| 4′-hydroxy, 2- | PubChem | chalconoid | 7.87 | 3.43 | 35.61 | 14.89 | 48 | |
| methoxychalcone | 5348419 | | | | | | | |
| 4′-hydroxy, 3- | PubChem | chalconoid | 7.87 | 3.43 | 36.22 | 14.23 | 49 | |
| methoxychalcone | 6123889 | | | | | | | |
| 4′-hydroxy, 4- | PubChem | chalconoid | 7.87 | 3.43 | 31.32 | 16.92 | 55 | |
| methoxychalcone | 5355594 | | | | | | | |
| 2,4′-dihydroxychalcone | PubChem | chalconoid | 7.83; 8.90 | 3.28 | 24.05 | 15.18 | 106 | |
| | 5861624 | | | | | | | |
| 2′,4′-dihydroxychalcone | 1776-30-3 | chalconoid | 7.43; 9.34 | 3.93 | 28.63 | 15.13 | 58 | |
| 3,4′-dihydroxychalcone | PubChem | chalconoid | 7.86; 9.42 | 3.28 | 25.22 | 15.25 | 96 | |
| | 8832859 | | | | | | | |
| 4,4′-dihydroxychalcone | 3600-61-1 | chalconoid | 7.84; 9.06 | 3.28 | 23.89 | 15.87 | 103 | |
| 2′,4′, dihydroxy, 2- | PubChem | chalconoid | 7.43; 9.34 | 3.78 | 34.57 | 14.92 | 47 | |
| methoxychalcone | 6161915 | | | | | | | |
| 2′,4′-dihydroxy, 4- | PubChem | chalconoid | 7.43; 9.34 | 3.78 | 30.96 | 16.94 | 52 | |
| methoxychalcone | 5711223 | | | | | | | |
| 2,2′,4′- | 26962-50-5 | chalconoid | 7.41; 8.75; | 3.63 | 31.96 | 15.14 | 64 | |
| trihydroxychalcone | | | 9.46 | | | | | |
| 4′- | PubChem | (dihydro)chalconoid | 7.78 | 3.51 | 37.31 | 14.40 | 48 | |
| hydroxydihydrochalcone | 14416163 | | | | | | | |
| 2′,4′-dihydroxy, 4- | PubChem | (dihydro)chalconoid | 7.34; 9.27 | 3.69 | 36.46 | 16.88 | 45 | |
| methoxydihydrochalcone | 578444 | | | | | | | |
| demethoxycapillarisin | 61854-36-2 | chromone | 7.22; 9.57; | 3.35 | 44.11 | 13.05 | 68 | |
| | | | 11.20 | | | | | |
| bolusanthol C | PubChem | flavonoid: | 7.07; 9.29; | 6.18 | 63.25/64.02 | 16.16/15.17 | 46/47 | |
| (stereoisomers 1/2) | 637199 | (iso)flavanone | 11.11 | | | | | |
| pinocembrin | 480-39-7 | flavonoid: | 7.28; 11.30 | 3.14 | 36.95/42.95 | 13.11/12.06 | 89/72 | ref. [32A] |
| (stereoisomers 1/2) | | (iso)flavanone | | | | | | |
| sigmoidin J (enol | 157999-01-4 | flavonoid: | 8.25; 9.38; | 3.75 | 49.80 | 16.70 | 62 | |
| tautomer) | | (iso)flavanone | 10.10 | | | | | |
| flavonol | 577-85-5 | flavonoid: flavonol | 5.29 | 2.72 | 33.07 | 13.50 | 62 | |
| 3-hydroxywogonin | PubChem | flavonoid: flavonol | 4.69; 8.00; | 2.61 | 43.08 | 13.52 | 42 | ref. [30A] |
| | 5378234 | | 12.04 | | | | | |
| kaempferide | PubChem | flavonoid: flavonol | 4.78; 7.49; | 2.61 | 37.74 | 15.53 | 63 | |
| | 5281666 | | 11.52 | | | | | |
| 5-methylflavonol | none | flavonoid: flavonol | 5.42 | 3.23 | 35.01 | 13.94 | 145 | |
| | available | | | | | | | |
| 6-methylflavonol | PubChem | flavonoid: flavonol | 5.39 | 3.23 | 33.99 | 14.58 | 151 | |
| | 227445 | | | | | | | |
| 7-methylflavonol | PubChem | flavonoid: flavonol | 5.43 | 3.23 | 38.27 | 13.89 | 121 | |
| | 1640313 | | | | | | | |
| 8-methylflavonol | none | flavonoid: flavonol | 5.40 | 3.23 | 38.61 | 13.50 | 125 | |
| | available | | | | | | | |
| 2′-methylflavonol | PubChem | flavonoid: flavonol | 5.41 | 3.23 | 37.79 | 13.65 | 129 | |
| | 44235774 | | | | | | | |
| 3′-methylflavonol | PubChem | flavonoid: flavonol | 5.39 | 3.23 | 36.72 | 13.72 | 136 | |
| | 44457131 | | | | | | | |
| 4′-methylflavonol | PubChem | flavonoid: flavonol | 5.42 | 3.23 | 33.61 | 14.42 | 153 | |
| | 265711 | | | | | | | |
| platanin | PubChem | flavonoid: flavonol | 4.73; 8.06; | 2.97 | 37.98 | 14.38 | 109 | ref. [30A] |
| | 627136 | | 12.27; 14.19 | | | | | |
| artelastin | 182052-05-7 | flavonoid: | 6.85; 8.42; | 7.06 | 77.70 | 15.79 | 50 | |
| | | (iso)flavone | 11.00 | | | | | |
| 7-hydroxyisoflavone | 13057-72-2 | flavonoid: | 7.48 | 3.03 | 29.48 | 14.36 | 81 | |
| | | (iso)flavone | | | | | | |
| 7-hydroxy-3- | 18651-15-5 | flavonoid: | 7.49 | 3.06 | 36.98 | 13.49 | 58 | |
| methylflavone | | (iso)flavone | | | | | | |
| 7-hydroxy-5- | 15235-99-1 | flavonoid: | 7.63 | 3.18 | 35.77 | 13.45 | 69 | |
| methylflavone | | (iso)flavone | | | | | | |
| 7-hydroxy-8- | PubChem | flavonoid: | 7.92 | 3.55 | 31.46 | 14.30 | 58 | |
| methylisoflavone | 5408595 | (iso)flavone | | | | | | |
| millewanin A | PubChem | flavonoid: | 7.26; 9.20; | 6.38 | 69.60 | 16.52 | 54 | |
| | 11154818 | (iso)flavone | 11.24 | | | | | |
| 6,8-diprenylgenistein | PubChem | flavonoid: | 6.89; 8.96; | 6.53 | 68.79 | 16.15 | 62 | |
| | 480783 | (iso)flavone | 11.04 | | | | | |
| 3′,5′-diprenylgenistein | PubChem | flavonoid: | 7.24; 8.59; | 6.53 | 68.02 | 16.32 | 66 | |
| | 44257287 | (iso)flavone | 11.24 | | | | | |
| apigenidin | 1151-98-0 | flavonoid: flavylium | 6.24; 7.93; | 3.18 | 35.90 | 14.12 | 373 | |
| | | cation | 8.67 | (protonated | | | | |
| | | | | sp.) | | | | |
| capensinidin | 19077-85-1 | flavonoid: flavylium | 5.98; 6.82; | 2.42 | 50.77 | 14.24 | 85 | |
| | | cation | 8.13 | (protonated | | | | |
| | | | | sp.) | | | | |
| diosmetinidin | 64670-94-6 | flavonoid: flavylium | 6.24; 7.93; | 2.93 | 37.71 | 14.27 | 188 | |
| | | cation | 8.69 | (protonated | | | | |
| | | | | sp.) | | | | |
| hursutidin | 4092-66-4 | flavonoid: flavylium | 5.92; 6.74; | 2.42 | 52.49 | 14.38 | 82 | |
| | | cation | 8.14 | (protonated | | | | |
| | | | | sp.) | | | | |
| 3-hydroxyflavylium | 7249-10-7 | flavonoid: flavylium | 5.72 | 3.75 | 33.00 | 13.49 | 185 | |
| | | cation | | (protonated | | | | |
| | | | | sp.) | | | | |
| 5-hydroxyflavylium | none | flavonoid: flavylium | 7.49 | 3.75 | 30.95 | 13.64 | 157 | |
| | available | cation | | (protonated | | | | |
| | | | | sp.) | | | | |
| 6-hydroxyflavylium | none | flavonoid: flavylium | 7.16 | 3.75 | 30.11 | 14.29 | 171 | ref. [28A] |
| | available | cation | | (protonated | | | | |
| | | | | sp.) | | | | |
| 7-hydroxyflavylium | ChemSpider | flavonoid: flavylium | 7.57 | 3.75 | 32.01 | 13.33 | 147 | |
| | 17612652 | cation | | (protonated | | | | |
| | | | | sp.) | | | | |
| 3-hydroxy, 6,4′- | none | flavonoid: flavylium | 5.81 | 3.24 | 39.87 | 17.09 | 319 | |
| dimethoxyflavylium | available | cation | | (protonated | | | | |
| | | | | sp.) | | | | |
| 3-hydroxy, 7,4′- | none | flavonoid: flavylium | 5.98 | 3.24 | 38.57 | 15.90 | 363 | |
| dimethoxyflavylium | available | cation | | (protonated | | | | |
| | | | | sp.) | | | | |
| 8-hydroxy, 7,4′- | none | flavonoid: flavylium | 5.41 | 3.24 | 38.90 | 15.90 | 336 | |
| dimethoxyflavylium | available | cation | | (protonated | | | | |
| | | | | sp.) | | | | |
| 3,6-dihydroxyflavylium | none | flavonoid: flavylium | 5.75; 7.05 | 3.46 | 33.54 | 14.14 | 752 | |
| | available | cation | | (protonated | | | | |
| | | | | sp.) | | | | |
| 3,6-dihydroxy, 4′- | none | flavonoid: flavylium | 5.79; 7.05 | 3.21 | 37.08 | 15.95 | 860 | |
| methoxyflavylium | available | cation | | (protonated | | | | |
| | | | | sp.) | | | | |
| luteolinidin | 1154-78-5 | flavonoid: flavylium | 6.24; 7.79; | 2.90 | 35.51 | 14.19 | 199 | |
| | | cation | 8.40; 11.71 | (protonated | | | | |
| | | | | sp.) | | | | |
| rosinidin | 4092-64-2 | flavonoid: flavylium | 5.94; 6.77; | 2.67 | 44.46 | 15.06 | 196 | |
| | | cation | 8.67 | (protonated | | | | |
| | | | | sp.) | | | | |
| vulpinic acid | 521-52-8 | furan | 5.38 | 3.23 | 47.12 | 15.52 | 74 | ref. [31A] |

Referring to Supplemental Data Table 1: Summary of 48 Compounds Tested.

Notes to Supplemental Data Table 1: Uncoupling activity measured in isolated rat mitochondria. Predicted proton shuttle uncoupling activity expressed in arbitrary units of rate of energy dissipation/mole uncoupler. * racemic mixture of 2 stereoisomers tested; predicted activity is an average of calculations performed for each stereoisomer. # number of distinct ionized species existing at mitochondrial pH too numerous for microspeciation calculation; the resistance-lowering effect of molecular distribution therefore not calculated. Acid-dissociation constants and octanol-water partition coefficients were calculated using ChemAxon Marvin 5.2. Minimal projection area and z-length perpendicular to this projected area were calculated using ChemAxon Marvin 5.3. For correlational analysis between measured and predicted activity, measurements inferior to 0% were transformed to 0%.

SUPPLEMENTAL DATA TABLE 1
Summary of 48 Compounds Tested.

| | | | | Calculated | | | Measured Uncoupling | Predicted |
|---|---|---|---|---|---|---|---|---|
| | | | | logP | Minimal | | Activity | Proton |
| | | | Calculated | (neutral | Projection | z-Length | (% uncoupling ± SEM | Shuttle Activity |
| Compound Name | CAS # | Chemical Class | pKa | species) | Area (Å²) | (Å) | @ 100 μM) | (×10⁶ a.u.) |
| anthraquinone | 84-65-1 | anthraquinone | n/a | 2.92 | 25.98 | 11.86 | −1 ± 1 | 0 |
| frangulic acid | 518-82-1 | anthraquinone | 7.39; 9.44; | 3.82 | 33.90 | 13.21 | 78 ± 14 | 74 |
| | | | 10.23 | | | | | |
| chalcone | 94-41-7 | chalconoid | n/a | 3.89 | 27.11 | 14.35 | −6 ± 0 | 0 |
| butein | 487-52-5 | chalconoid | 7.75; 8.77; | 3.33 | 30.57 | 15.32 | 71 ± 4 | 83 |
| | | | 9.35; 12.46 | | | | | |
| homobutein | 34000- | chalconoid | 7.77; 8.96; | 3.47 | 36.15 | 15.42 | 56 ± 5 | 56 |
| | 39-0 | | 9.65 | | | | | |
| 4′-hydroxychalcone | 2657-25-2 | chalconoid | 7.87 | 3.59 | 26.27 | 15.07 | 74 ± 14 | 81 |
| isoliquiritigenin | 961-29-5 | chalconoid | 7.75; 8.78; | 3.63 | 28.74 | 15.54 | 108 ± 7 | 77 |
| | | | 9.36 | | | | | |
| phloretin | 60-82-2 | (dihydro)chalconoid | 8.00; 9.49; | 3.90 | 38.66 | 15.17 | 15 ± 3 | 39 |
| | | | 10.68; | | | | | |
| | | | 11.96 | | | | | |
| caffeic acid | 331-39-5 | cinnamate | 3.64; 9.28; | 1.53 | 25.37 | 12.06 | 0 ± 1 | 0 |
| | | | 12.69 | | | | | |
| ferulic acid | 1135-24-6 | cinnamate | 3.77; 9.98 | 1.67 | 29.88 | 12.12 | −1 ± 1 | 0 |
| (+) catechin | 154-23-4 | flavonoid: flavanol | 9.00; 9.62; | 1.80 | 46.81 | 12.15 | −2 ± 0 | 0 |
| | | | 10.80; | | | | | |
| | | | 12.65; | | | | | |
| | | | 14.09 | | | | | |
| (−) epigallocatechin | 989-51-5 | flavonoid: flavanol | 7.99; 8.64; | 3.08 | 62.85 | 13.67 | 0 ± 1 | 1 (#) |
| gallate | | | 9.09; | | | | | |
| | | | 10.39; | | | | | |
| | | | 11.60; | | | | | |
| | | | 12.83; | | | | | |
| | | | 13.31 | | | | | |
| silibinin (A and B) | 22888- | flavonoid: | 7.81; 9.83; | 2.63 | 73.12/70.74 | 14.07/15.77 | 5 ± 1 | 4 (*#) |
| | 70-6 | (iso)flavanone | 10.62; | | | | | |
| | | | 12.28; | | | | | |
| | | | 14.56 | | | | | |
| (±) hesperetin | 69097- | flavonoid: | 7.92; 9.74; | 2.68 | 39.35/48.53 | 15.60/12.94 | 5 ± 1 | 21 (*) |
| | 99-0 | (iso)flavanone | 10.71 | | | | | |
| (±) naringenin | 480-40-1 | flavonoid: | 7.91; 9.46; | 2.84 | 36.81/44.30 | 14.11/11.58 | 4 ± 2 | 38 (*) |
| | | (iso)flavanone | 10.68 | | | | | |
| datiscetin | 480-15-9 | flavonoid: flavonol | 4.45; 6.84; | 2.46 | 37.56 | 13.62 | 33 ± 3 | 21 |
| | | | 9.11; 9.75 | | | | | |
| galangin | 548-83-4 | flavonoid: flavonol | 4.79; 6.85; | 2.76 | 35.84 | 13.51 | 95 ± 9 | 101 |
| | | | 9.57 | | | | | |
| kaempferol | 520-18-3 | flavonoid: flavonol | 4.70; 6.84; | 2.46 | 36.86 | 14.12 | 25 ± 2 | 49 |
| | | | 9.28; 9.88 | | | | | |
| morin | 480-16-0 | flavonoid: flavonol | 4.45; 6.84; | 2.16 | 41.24 | 13.51 | 1 ± 0 | 9 |
| | | | 9.16; 9.76; | | | | | |
| | | | 10.84 | | | | | |
| myricetin | 529-44-2 | flavonoid: flavonol | 4.40; 6.84; | 1.85 | 39.39 | 14.33 | 11 ± 0 | 4 |
| | | | 8.91; 9.67; | | | | | |
| | | | 11.33; | | | | | |
| | | | 14.71 | | | | | |
| quercetin | 117-39-5 | flavonoid: flavonol | 4.54; 6.84; | 2.16 | 36.77 | 14.28 | 8 ± 1 | 13 |
| | | | 9.12; 9.80; | | | | | |
| | | | 12.82 | | | | | |
| flavone | 525-82-6 | flavonoid: | n/a | 2.97 | 32.19 | 13.36 | −1 ± 2 | 0 |
| | | (iso)flavone | | | | | | |
| apigenin | 520-36-5 | flavonoid: | 6.63; 8.63; | 2.71 | 35.89 | 14.12 | 19 ± 3 | 32 |
| | | (iso)flavone | 9.42 | | | | | |
| biochanin A | 491-80-5 | flavonoid: | 6.61; 9.29 | 3.22 | 35.28 | 16.19 | 103 ± 22 | 91 |
| | | (iso)flavone | | | | | | |
| chrysin | 480-40-0 | flavonoid: | 6.64; 9.32 | 3.01 | 35.04 | 13.51 | 137 ± 16 | 70 |
| | | (iso)flavone | | | | | | |
| daidzein | 486-66-8 | flavonoid: | 6.48; 8.96 | 2.73 | 29.59 | 14.96 | 6 ± 1 | 39 |
| | | (iso)flavone | | | | | | |
| formononetin | 485-72-3 | flavonoid: | 6.48 | 2.88 | 34.67 | 16.12 | 24 ± 1 | 37 |
| | | (iso)flavone | | | | | | |
| genistein | 446-72-0 | flavonoid: | 6.61; 8.79; | 3.08 | 31.91 | 14.98 | 74 ± 4 | 92 |
| | | (iso)flavone | 9.46 | | | | | |
| amentoflavone | 1617-53-4 | flavonoid: bis- | 6.12; 6.78; | 5.09 | 73.90 | 18.93 | 4 ± 1 | 3 (#) |
| | | flavonoid | 8.12; 8.72; | | | | | |
| | | | 9.15; 9.76 | | | | | |
| cupressuflavone | 3952-18-9 | flavonoid: bis- | 5.84; 6.37; | 5.09 | 83.38 | 13.19 | −2 ± 0 | 3 (#) |
| | | flavonoid | 8.33; 8.65; | | | | | |
| | | | 8.82; 9.51 | | | | | |
| sciadopitysin | 521-34-6 | flavonoid: bis- | 6.28; 8.49; | 5.53 | 79.80 | 17.46 | −1 ± 0 | 1 (#) |
| | | flavonoid | 9.27 | | | | | |
| phenol | 108-95-2 | simple phenolic | 10.02 | 1.67 | 19.41 | 7.90 | 0 ± 0 | 1 |
| benzoic acid | 65-85-0 | simple phenolic | 4.08 | 1.63 | 19.20 | 9.43 | 0 ± 1 | 0 |
| carvacrol | 499-75-2 | simple phenolic | 10.42 | 3.43 | 30.26 | 10.40 | 7 ± 2 | 9 |
| catechol | 120-80-9 | simple phenolic | 9.34; 12.79 | 1.37 | 22.61 | 7.92 | 0 ± 0 | 0 |
| gallic acid | 149-91-7 | simple phenolic | 3.94; 9.04; | 0.72 | 26.38 | 10.04 | −2 ± 1 | 0 |
| | | | 11.17; | | | | | |
| | | | 14.80 | | | | | |
| hydroquinone | 123-31-9 | simple phenolic | 9.68; 11.55 | 1.37 | 21.67 | 8.31 | 0 ± 1 | 0 |
| 4-acetylphenol | 99-93-4 | simple phenolic | 7.79 | 1.23 | 21.41 | 10.32 | 0 ± 0 | 2 |
| 7-hydroxychromone | 59887- | simple phenolic | 6.53 | 1.37 | 25.06 | 9.93 | 0 ± 0 | 3 |
| | 89-7 | | | | | | | |
| pyrogallol | 87-66-1 | simple phenolic | 8.94; | 1.06 | 23.08 | 7.90 | 1 ± 1 | 0 |
| | | | 11.30; | | | | | |
| | | | 14.70 | | | | | |
| resorcinol | 108-46-3 | simple phenolic | 9.26; 10.73 | 1.37 | 21.64 | 7.82 | 0 ± 0 | 1 |
| salicylic acid | 69-72-7 | simple phenolic | 2.79; 13.23 | 1.98 | 22.16 | 9.12 | 4 ± 0 | 0 |
| thymol | 89-83-8 | simple phenolic | 10.59 | 3.43 | 32.24 | 10.44 | 6 ± 0 | 7 |
| vanillin | 121-33-5 | simple phenolic | 7.81 | 1.22 | 27.99 | 9.33 | 0 ± 1 | 1 |
| trans-stilbene | 103-30-0 | stilbenoid | n/a | 4.31 | 23.82 | 13.71 | −1 ± 1 | 0 |
| trans-piceatannol | 4339-71-3 | stilbenoid | 8.91; 9.49; | 3.10 | 29.29 | 14.49 | 5 ± 1 | 10 |
| | | | 10.62; | | | | | |
| | | | 12.68 | | | | | |
| trans-pinosylvin | 102-61-4 | stilbenoid | 9.16; 10.61 | 3.71 | 27.80 | 13.73 | 3 ± 3 | 26 |
| trans-resveratrol | 501-36-0 | stilbenoid | 8.99; 9.63; | 3.40 | 26.81 | 14.49 | 2 ± 0 | 21 |
| | | | 10.64 | | | | | |

Referring to Supplemental Data Table 2: Summary of Proposed Monoprotic and Diprotic Derivatives Optimized for Activity.

Notes to Supplemental Data Table 2: Predicted proton shuttle uncoupling activity expressed in arbitrary units of rate of energy dissipation/mole. Compounds have been screened in-silico only and have not been synthesized and tested. Compounds have no CAS registry number, PubChem CID, or ChemSpider ID. Acid-dissociation constants and octanol-water partition coefficients were calculated using ChemAxon Marvin 5.2. Minimal projection area and z-length perpendicular to this projected area were calculated using ChemAxon Marvin 5.3

SUPPLEMENTAL DATA TABLE 2
Summary of Proposed Monoprotic and Diprotic Derivatives Optimized for Activity.

| | | | | | | Predicted Activity | Predicted |
|---|---|---|---|---|---|---|---|
| | | | | | | of | Activity of |
| | | Calculated | Calculated | Minimal | | Monoprotic | Diprotic |
| | | pKa₍₁₎; | logP | Projection | z-Length | Species | Species |
| Chemical Class | SMILES String Structure | pka₍₂₎ | (neutral/ionized) | Area (Å2) | (Å) | (×10⁶ a.u.) | (×10⁶ a.u.) |
| 2-hydroxyanthraquinone | CC(═O)C1═C(O)C(C(C)═O)═C2C(═O)C3═C(C═CC═C3)C(═O)C2═C1 | 4.87 | 3.03/0.68 | 37.72 | 12.77 | 224 | n/a |
| 3-hydroxybenzofuran | CC1═CC═C2OC(C(═O)C═C)═C(O)C2═C1 | 6.20 | 3.23/1.42 | 25.72 | 13.13 | 484 | n/a |
| | CC(═O)C1═C(O)C2═CC(C═C)═C(C)C═C2O1 | 5.97 | 3.21/1.42 | 29.71 | 12.60 | 433 | n/a |
| | C\C═C\C(═O)C1═C(O)C2═CC═CC═C2O1 | 5.89 | 3.10/1.32 | 27.52 | 13.54 | 392 | n/a |
| | C\C═C\C1═CC═C2OC(C(C)═O)═C(O)C2═C1 | 5.89 | 3.08/1.30 | 26.87 | 14.38 | 370 | n/a |
| | C\C═C\C1═CC═C2OC(C(C)═O)═C(O)C2═C1 | 5.89 | 3.08/1.30 | 26.91 | 14.38 | 369 | n/a |
| | OC1═C(OC2═CC═C(C═C12)C(═O)C═C)C(═O)C═C | 5.06 | 3.03/1.30 | 29.23 | 15.25 | 351 | n/a |
| | C\C═C\C(═O)C1═C(O)C2═CC(C(C)═O)═C(C)C═C2O1 | 5.06 | 3.17/1.44 | 34.44 | 15.82 | 340 | n/a |
| | C\C═C\C(═O)C1═C(C)C═C2OC(C(C)═O)═C(O)C2═C1 | 5.04 | 3.17/1.45 | 35.99 | 14.91 | 331 | n/a |
| | C\C(C)═C\C1═CC═C2OC(C(C)═O)═C(O)C2═C1 | 5.87 | 3.32/1.54 | 30.53 | 14.45 | 315 | n/a |
| | CC(═O)C1═C(O)C2═CC(C)═C(C)C═C2O1 | 6.17 | 2.98/1.18 | 27.66 | 12.08 | 291 | n/a |
| | CC(C)═CC(═O)C1═C(O)C2═CC(═CC═C2O1)C(C)═O | 4.99 | 2.90/1.18 | 29.02 | 15.49 | 267 | n/a |
| 3-hydroxychromone | CC1═CC2═C(C(C)═C1)C(═O)C(O)═C(O2)C(═O)C═C | 4.05 | 3.16/−0.37 | 30.87 | 12.61 | 406 | n/a |
| | CC1═CC2═C(C═C1C)C(═O)C(O)═C(O2)C(═O)C═C | 4.03 | 3.16/−0.37 | 30.92 | 13.25 | 385 | n/a |
| | C\C(C)═C\C(═O)C1═C(O)C(═O)C2═C(O1)C═C(C)C═C2 | 4.07 | 3.28/−0.25 | 36.90 | 14.15 | 245 | n/a |
| | CC(═O)C1═C(O)C(═O)C2═C(O1)C═C(C)C(C═C)═C2C | 4.01 | 3.14/−0.39 | 37.63 | 13.63 | 241 | n/a |
| | CC1═C(O)C(═O)C2═C(O1)C(C)═C(C)C(C)═C2 | 5.38 | 3.16/−0.36 | 33.14 | 11.39 | 192 | n/a |
| | CC1═C(O)C(═O)C2═C(O1)C═C(C═C)C(C═C)═C2 | 5.19 | 3.10/−0.43 | 33.77 | 12.10 | 173 | n/a |
| | C\C═C\C(═O)C1═CC2═C(C═C1\C═C\C)C(═O)C(O)═CO2 | 4.99 | 3.25/−0.28 | 41.68 | 12.39 | 146 | n/a |
| | C\C═C\C1═CC2═C(C(═O)C(O)═CO2)C(C)═C1 | 5.40 | 3.06/−0.47 | 32.08 | 13.17 | 138 | n/a |
| | C\C═C\C1═CC2═C(OC(C═C)═C(O)C2═O)C═C1 | 5.60 | 3.12/−0.41 | 31.24 | 14.78 | 128 | n/a |
| | CC1═CC(C)═C2C(═O)C(O)═COC2═C1C | 5.49 | 2.96/−0.57 | 32.89 | 11.17 | 115 | n/a |
| 7-hydroxychromone | CC(═O)C1═C(O)C(═CC2═C1OC(C)═C(C)C2═O)C(═O)C═C | 5.05 | 3.14/0.78 | 36.15 | 13.46 | 278 | n/a |
| | C\C═C\C1═C(C)C(═O)C2═C(O1)C(C(C)═O)═C(O)C(═C2)C(C)═O | 4.97 | 3.14/0.79 | 38.94 | 14.28 | 233 | n/a |
| | C\C═C\C1═CC(═O)C2═C(O1)C(C═C)═C(O)C(═C2)C(C)═O | 6.20 | 3.27/1.05 | 34.18 | 14.71 | 190 | n/a |
| | CC1═C(C)C(═O)C2═C(O1)C(C═O)═C(O)C(C═C)═C2 | 6.24 | 3.06/0.84 | 35.08 | 11.14 | 184 | n/a |
| | OC1═C(C═C)C2═C(C═C1C(═O)C═C)C(═O)C═CO2 | 6.29 | 3.07/0.84 | 33.78 | 12.21 | 179 | n/a |
| | CC1═C(C)C(═O)C2═C(O1)C(C═C)═C(O)C(C═O)═C2 | 6.25 | 3.06/0.84 | 35.82 | 11.11 | 176 | n/a |
| | CC(═O)C1═CC2═C(OC(C═C)═C(C)C2═O)C(C═C)═C1O | 6.18 | 3.28/1.06 | 37.30 | 13.27 | 176 | n/a |
| | OC1═C(C═O)C2═C(C═C1C═C)C(═O)C(C═C)═CO2 | 6.22 | 3.00/0.78 | 31.74 | 12.66 | 174 | n/a |
| | CC(═O)C1═CC2═C(OC(C═C)═C(C═C)C2═O)C(C)═C1O | 6.54 | 3.20/0.95 | 36.24 | 12.60 | 171 | n/a |
| | OC1═C(C═C)C2═C(C═C1C═O)C(═O)C(C═C)═CO2 | 6.24 | 3.00/0.78 | 32.49 | 12.73 | 164 | n/a |
| | C\C═C\C(═O)C1═C(O)C═CC2═C1OC═C(C)C2═O | 6.37 | 3.11/0.88 | 36.37 | 12.20 | 162 | n/a |
| | C\C═C\C1═C(C)C(═O)C2═C(O1)C═C(O)C(═C2)C(C)═O | 6.11 | 2.93/0.72 | 30.71 | 14.38 | 149 | n/a |
| | C\C═C\C1═CC2═C(OC═C(C)C2═O)C(C(C)═O)═C1O | 6.17 | 3.09/0.87 | 39.67 | 12.88 | 139 | n/a |
| | C\C═C\C1═C(C)C(═O)C2═C(O1)C(C(C)═O)═C(O)C═C2 | 6.28 | 2.93/0.70 | 38.79 | 11.25 | 107 | n/a |
| | C\C═C\C(═O)C1═CC2═C(OC(C)═CC2═O)C═C1O | 6.20 | 2.92/0.70 | 34.98 | 14.45 | 105 | n/a |
| | C\C═C\C1═C(O)C(═CC2═C1OC(C)═CC2═O)C(C)═O | 6.19 | 2.90/0.68 | 37.26 | 12.66 | 102 | n/a |
| 3-hydroxycoumarin | C\C(C)═C\C1═CC2═C(OC(═O)C(O)═C2C)C═C1 | 4.90 | 3.20/−0.33 | 31.44 | 13.08 | 273 | n/a |
| | C\C(C)═C\C1═CC2═C(C═C1)C(C)═C(O)C(═O)O2 | 4.92 | 3.20/−0.33 | 32.98 | 13.40 | 242 | n/a |
| | CC1═C(C)C(C)═C2OC(═O)C(O)═CC2═C1 | 5.13 | 3.08/−0.45 | 30.81 | 10.74 | 232 | n/a |
| | CC(═O)C1═C(C)C(C═C)═C(C)C2═C1C(C)═C(O)C(═O)O2 | 4.59 | 3.16/−0.37 | 42.40 | 11.13 | 188 | n/a |
| | C\C═C\C1═CC═C2C(OC(═O)C(O)═C2C)═C1 | 4.85 | 2.96/−0.57 | 29.94 | 13.21 | 177 | n/a |
| | C\C═C(/C)C1═CC2═C(C═C1)C═C(O)C(═O)O2 | 4.99 | 2.96/−0.57 | 28.40 | 13.46 | 177 | n/a |
| | C\C═C\C(═O)C1═CC2═C(C(C)═C1)C(C═C)═C(O)C(═O)O2 | 4.79 | 3.19/−0.34 | 39.70 | 15.10 | 155 | n/a |
| 4-hydroxycoumarin | OC1═C(C(═O)C═C)C(═O)OC2═C1C(C═C)═CC(C═C)═C2 | 5.52 | 3.18/0.35 | 35.42 | 14.80 | 190 | n/a |
| | CC(C)═CC1═CC2═C(C═C1)C(O)═C(C(═O)C═C)C(═O)O2 | 5.68 | 3.07/0.24 | 34.77 | 15.21 | 136 | n/a |
| | OC1═CC(═O)OC2═C1C═CC(═C2)C(═O)\C═C\C1═CC═CC═C1 | 5.74 | 2.95/0.12 | 29.29 | 16.31 | 132 | n/a |
| | C\C═C\C1═CC2═C(C(═C1)\C═C\C)C(O)═C(C(C)═O)C(═O)O2 | 5.53 | 3.19/0.36 | 45.06 | 14.66 | 120 | n/a |
| 6-hydroxy-1,4- | C\C═C\C1═C(/C═C/C)C(═O)C2═CC(C(C)═O)═C(O)C═C2C1═O | 5.99 | 3.24/1.04 | 39.82 | 14.63 | 164 | n/a |
| naphtoquinone | | | | | | | |
| | CC(═O)C1═CC2═C(C═C1O)C(═O)C(═CC2═O)C1═CC═CC═C1 | 6.03 | 3.07/0.86 | 30.17 | 16.11 | 198 | n/a |
| | CC(═O)C1═CC2═C(C═C1O)C(═O)C═C(C1═CC═CC═C1)C2═O | 6.00 | 3.07/0.86 | 33.96 | 15.12 | 169 | n/a |
| | OC1═CC2═C(C═C1C═O)C(═O)C═C(C2═O)C1═CC═CC═C1 | 6.08 | 3.22/1.01 | 27.43 | 15.23 | 324 | n/a |
| | OC1═CC2═C(C═C1C═O)C(═O)C(═CC2═O)C1═CC═CC═C1 | 6.06 | 3.22/1.01 | 30.86 | 14.59 | 270 | n/a |
| | CC(═O)C1═C(O)C(C)═C2C(═O)C(C)═C(C)C(═O)C2═C1C | 6.60 | 3.22/0.97 | 38.77 | 12.33 | 145 | n/a |
| | C\C═C\C1═C(C)C(═O)C2═CC(C(C)═O)═C(O)C(C)═C2C1═O | 6.48 | 3.23/0.99 | 36.73 | 13.96 | 153 | n/a |
| | C\C═C\C1═C(C)C(═O)C2═C(C)C(O)═C(C═C2C1═O)C(C)═O | 6.47 | 3.23/0.99 | 40.74 | 13.60 | 128 | n/a |
| | CC(═O)C1═C2C(═O)C(C)═C(C)C(═O)C2═C(C)C(C)═C1O | 6.61 | 3.22/0.97 | 43.88 | 11.37 | 121 | n/a |
| | CC(═O)C1═C2C(═O)C(C═C)═C(C═C)C(═O)C2═C(C═C)C═C1O | 6.04 | 3.21/1.00 | 48.93 | 12.26 | 131 | n/a |
| | OC1═C(C═O)C2═C(C═C1)C(═O)C═C(C2═O)C1═CC═CC═C1 | 6.18 | 3.22/1.00 | 36.08 | 13.52 | 199 | n/a |
| | OC1═C(C═O)C2═C(C═C1)C(═O)C(═CC2═O)C1═CC═CC═C1 | 6.16 | 3.22/1.00 | 25.94 | 14.62 | 361 | n/a |
| | CC(═O)C1═CC2═C(C(═O)C(═CC2═O)C2═CC═CC═C2)C(C(C)═O)═C1O | 4.90 | 3.27/0.93 | 43.54 | 15.89 | 175 | n/a |
| | CC(═O)C1═CC2═C(C(═O)C═C(C3═CC═CC═C3)C2═O)C(C(C)═O)═C1O | 4.87 | 3.27/0.93 | 36.96 | 15.00 | 262 | n/a |
| | CC(═O)C1═C(C)C2═C(C(═O)C(C═C)═C(C═C)C2═O)C(C(C)═O)═C1O | 4.97 | 3.19/0.84 | 44.54 | 13.12 | 218 | n/a |
| | OC1═C(C═O)C2═C(C(═O)C(C═C)═CC2═O)C(C═C)═C1C═O | 4.98 | 3.19/0.83 | 39.03 | 12.43 | 294 | n/a |
| | C\C═C\C1═CC(═O)C2═C(C1═O)C(C═C)═C(C(C)═O)C(O)═C2C(C)═O | 4.88 | 3.27/0.92 | 46.32 | 13.64 | 180 | n/a |
| simple phenolic | C\C═C\C(═O)C1═CC(C═O)═C(O)C(C═O)═C1 | 5.38 | 3.10/0.71 | 32.70 | 12.70 | 284 | n/a |
| | CC(═O)C1═CC(C(═O)C═C)═C(O)C(═C1)C(═O)C═C | 5.31 | 3.16/0.78 | 35.00 | 13.62 | 276 | n/a |
| | CC(C)═CC(═O)C1═CC(C(C)═O)═C(O)C(═C1)C(C)═O | 5.27 | 3.03/0.66 | 40.66 | 12.80 | 165 | n/a |
| | CCCCCC(═O)C1═CC═C(O)C═C1 | 7.78 | 3.26/1.15 | 27.43 | 15.16 | 92 | n/a |
| 3-hydroxychromenylium | CC1═CC2═C(C═C1C)[O+]═CC(O)═C2 | 5.32 | 3.13/1.80 | 24.36 | 11.03 | 1248 | n/a |
| | CC1═CC2═C(C═C1C)[O+]═C(C)C(O)═C2 | 6.10 | 3.25/1.92 | 28.53 | 11.29 | 908 | n/a |
| | OC1═CC2═C(C═CC(C═C)═C2)[O+]═C1 | 4.99 | 3.10/1.76 | 25.73 | 11.25 | 1028 | n/a |
| | CC1═[O+]C2═C(C═C(C═C)C═C2)C═C1O | 5.78 | 3.21/1.88 | 26.96 | 12.60 | 1011 | n/a |
| | OC1═CC2═C(C═C(C═C)C═C2)[O+]═C1 | 5.05 | 3.10/1.76 | 23.05 | 11.76 | 1225 | n/a |
| | CC1═[O+]C2═C(C═CC(C═C)═C2)C═C1O | 5.85 | 3.21/1.88 | 25.12 | 11.79 | 1241 | n/a |
| 4-hydroxychromenylium | OC1═CC═[O+]C2═C1C═CC(C═C)═C2 | 6.87 | 3.10/1.76 | 24.70 | 11.53 | 961 | n/a |
| | CC1═[O+]C2═C(C═CC(C═C)═C2)C(O)═C1 | 7.10 | 3.21/1.88 | 29.19 | 11.78 | 799 | n/a |
| 8-hydroxychromenylium | OC1═C(C═C)C═CC2═C1[O+]═CC═C2 | 4.82 | 3.10/0.51 | 24.04 | 11.85 | 1078 | n/a |
| | CC1═[O+]C2═C(C═C1)C═CC(C═C)═C2O | 4.91 | 3.21/0.63 | 26.02 | 12.51 | 1070 | n/a |
| dihydroxyanthraquinone | OC1═CC2═C(C═C1C═O)C(═O)C1═CC(O)═C(C═O)C═C1C2═O | 5.75; 6.35 | 3.04/0.83; −1.40 | 34.81 | 14.19 | 188 | 354 |
| dihydroxybenzofuran | CC1═C(O)C2═C(C(O)═C(O2)C═C)C(C═O)═C1C═C | 5.45; 6.87 | 3.14/1.59; 1.16; | 33.36 | 12.92 | 362/281 | 812 |
| | | | 0.00 | | | | |
| | CC(C)═CC1═C(O)C2═C(O1)C(O)═C(C═C)C═C2C(C)═O | 5.28; 6.92 | 3.11/1.11; 1.58; −0.05 | 38.62 | 14.24 | 265/178 | 565 |
| | C\C═C\C(═O)C1═CC(C═C)═C(O)C2═C1C(O)═CO2 | 5.14; 6.89 | 2.96/0.97; 1.44; −0.19 | 36.76 | 11.92 | 248/159 | 529 |
| | CC(C)═CC1═C(O)C2═C(O1)C(O)═C(C═C2)C(C)═O | 5.57; 7.24 | 3.02/0.79; 1.47; −0.38 | 29.96 | 14.41 | 303/163 | 497 |
| | CC(═O)C1═C(O)C2═C(C(O)═C(O2)C═C)C(C═C)═C1 | 4.64; 8.21 | 3.13/1.45; 1.01; −0.83 | 33.54 | 13.39 | 383/127 | 396 |
| dihydroxychromone | CC(═O)C1═C(O)C═C2OC3═C(C(C)═O)C(O)═C(C)C═C3C(═O)C2═C1 | 5.58; 6.33 | 3.28/1.09; 1.06; −1.16 | 35.81 | 14.57 | 211/164 | 425 |
| | CC(═O)C1═C2OC3═C(C(C)═O)C(O)═C(C)C═C3C(═O)C2═CC═C1O | 5.70; 6.37 | 3.28/1.08; 1.06; −1.16 | 38.37 | 13.34 | 200/170 | 404 |
| | CC1═C2OC(C═C)═C(O)C(═O)C2═CC(C(═O)C═C)═C1O | 5.24; 6.74 | 3.17/−0.36; 0.93; | 32.56 | 14.13 | 171/180 | 208 |
| | | | −2.61 | | | | |
| dihydroxyphenol | CC(═O)C1═CC(═CC═C1O)C(═O)C1═CC(C(C)═O)═C(O)C═C1 | 5.81; 6.41 | 3.24/1.03; −1.20 | 33.96 | 14.98 | 229 | 412 |
| | OC1═CC(O)═C(C═C1C(═O)C═C)C(═O)C═C | 6.03; 7.87 | 3.29/1.06; −1.20 | 33.23 | 11.58 | 269 | 189 |
| | CC(═O)C1═C(O)C(C═C)═C(O)C(C(C)═O)═C1C═C | 5.89; 7.58 | 3.25/1.03; −1.22 | 42.85 | 9.81 | 217 | 212 |
| dihydroxychromenylium | CC1═C(C)C2═C(C═C(O)C═[O+]2)C(C)═C1O | 5.79; 7.49 | 3.32/1.98; 0.73; −0.60 | 34.39 | 10.62 | 577/348 | 1097 |
| | C\C═C\C1═CC2═C(C═C(O)C(C)═[O+]2)C═C1O | 5.84; 6.57 | 3.28/1.94; 0.69; −0.64 | 33.60 | 13.11 | 533/432 | 1261 |
| | CC1═C(O)C(C═C)═CC2═C1C═C(O)C═[O+]2 | 5.50; 6.62 | 3.28/1.95; 0.70; −0.64 | 30.95 | 12.13 | 688/536 | 1712 |
| 2-hydroxyanthraquinone | CC(═O)C1═C(O)C(C(C)═O)═C2C(═O)C3═C(C═CC═C3)C(═O)C2═C1 | 4.87 | 3.03/0.68 | 37.72 | 12.77 | 224 | n/a |

Referring to Documents: [1A] M. D. Brand et al., Biochem. J. 392 (2005) 353-362. [2A] V. P. Skulachev, Biochim. Biophys. Acta 1363 (1998) 100-124. [3A] C. Affourtit, et al. Biochem. J. 409 (2008) 199-204. [4A] K. B. Wallace, et al. Ann. Rev. Pharmacol. Toxicol. 40 (2000) 353-388. [5A] W. F. Loomis, et al. J. Biol. Chem. 173 (1948) 807. [6A] P. Mitchell, Nature 191 (1961) 144-148. [7A] S. G. McLaughlin, et al., Physiol. Rev. 60 (1980) 825-863. [8A] H. Terada, Environ. Health Perspect. 87 (1990) 213-218. [9A] W. G. Whittingham, Uncouplers of oxidative phosphorylation, in: W. Kramer, U. Schirmer (Eds.), Modern crop protection compounds, Wiley, Weinheim, 2007, pp. 505-528. [10A] P. H. Lou, et al., Biochem. J. 407 (2007) 129-140. [11A] H. P. Ting, et al., Arch. Biochem. Biophys. 141 (1970) 141-146. [12A] L. C. Martineau, Biochim. Biophys. Acta 1820 (2012) 133-150. [13A] P. Misra, Expert Opin. Ther. Targets 12 (2008) 91-100. [14A] B. Viollet, et al., Front. Biosci. 14 (2009) 3380-3400. [15A] G. R. Steinberg, Physiol. Rev. 89 (2009) 1025-1078. [16A] L. C. Martineau, et al., J. Ethnopharmacol. 127 (2010) 396-406. [17A] A. Benhaddou-Andaloussi, et al., Diabetes Obes. Metab. 12 (2010) 148-157. [18A] H. M. Eid, et al., Biochem. Pharmacol. 79 (2010) 444-454. [19A] J. Manchester, et al., J. Chem. Info. Model. (doi: 10.1021/ci100019p) (2010). [20A] V. N. Viswanadhan, et al. J. Chem. Info. Model. 29 (1989) 163-172. [21A] A. Avdeef, Absorption and drug development: solubility, permeability, and charge state, Wiley-Interscience, Hoboken, N.J., 2003. [22A] E. H. C. Verhaeren, Phytochemistry 19 (1980) 501-503. [23A] C. van Dijk, et al., Biochem. Pharmacol. 60 (2000) 1593-1600. [24A] H. Nagamune, et al., Biochim. Biophys. Acta 1141 (1993) 231-237. [25A] P. Ravanel, et al., Phytochem. 21 (1982) 2845-2850. [26A] B. Inoue, et al., J. Toxicol. Sci 7 (1982) 245-254. [27A] D. J. Dorta, et al., Chem. Biol. Interact. 152 (2005) 67-78. [28A] G. Stenlid, Phytochemistry 9 (1970) 2251-2256. [29A] K. Kawai, et al., Cell Biol. Toxicol. 2 (1986) 457-467. [30A] S. Creuzet, et al., Phytochemistry 27 (1988) 3093-3099. [31A] A. N. Abo-Khatwa, et al., Nat. Toxins 4 (1996) 96-102. [32A] A. C. Santos, et al., Free Radical Biol. Medic. 24 (1998) 1455-1461. [33A] B. I. Escher, et al., Env. Sci. Tech. 30 (1996) 3071-3079. [34A] B. I. Escher, et al., Env. Sci. Tech. 33 (1999) 560-570. [35A] S. Spycher, et al., Chem. Res. Toxicol. 18 (2005) 1858-1867. [36A] S. Spycher, et al., Chem. Res. Toxicol. 21 (2008) 911-927. [37A] A. W. Barstad, et al., Biochim. Biophys. Acta 1140 (1993) 262-270. [38A] A. A. Starkov, et al., Biochim. Biophys. Acta 1318 (1997) 159-172. [39A] J. Parascandola, Mol. Cell. Biochem. 5 (1974) 69-77. [40A] W. D. Horner, Arch Ophthalmol 27 (1942) 1097-1121. [41A] J. A. Harper, et al., Rev. 2 (2001) 255-265. [42A] M. E. Harper, et al., Annu. Rev. Nutr. 28 (2008) 13-33. [43A] D. G. Hardie, Endocrinology 144 (2003) 5179-5183. [44A] W. W. Winder, et al., Cell Biochem. Biophys. 47 (2007) 332-347. [45A] G. Zhou, et al., J. Clin. Invest 108 (2001) 1167-1174. [46A] M. R. Owen, et al., Biochem. J. 348 Pt 3 (2000) 607-614. [47A] P. Fouqueray, et al., J. Diabetes Metab. 2:126 doi:10.4172/2155-6156.1000126 (2011). [48A] T. Hayashi, et al., Diabetes 49 (2000) 527-531.

Large enhancement of skeletal muscle cell glucose uptake and suppression of hepatocyte glucose-6-phosphatase activity by weak uncouplers of oxidative phosphorylation (Martineau, Biochimica et Biophysica Acta, 1820: 133-150; 2012)

Background: Perturbation of energy homeostasis in skeletal muscle and liver resulting from a transient inhibition of mitochondrial energy transduction can produce effects of relevance for the control of hyperglycemia through activation of the AMP-activated protein kinase, as exemplified by the antidiabetic drug metformin. The present focuses on uncoupling of oxidative phosphorylation rather than its inhibition as a trigger for such effects. Methods: The reference weak uncoupler 2,4-dinitrophenol, fourteen naturally-occurring phenolic compounds identified as uncouplers in isolated rat liver mitochondria, and fourteen related compounds with little or no uncoupling activity were tested for enhancement of glucose uptake in differentiated C2C12 skeletal muscle cells following 18 h of treatment at 25-100 μM. A subset of compounds were tested for suppression of glucose-6-phosphatase (G6Pase) activity in H4IIE hepatocytes following 16 h at 12.5-25 μM. Metformin (400 μM) was used as a standard in both assays. Results: Dinitrophenol and nine of eleven compounds that induced 50% or more uncoupling at 100 μM in isolated mitochondria enhanced basal glucose uptake by 53 to 269%; the effect of the 4′-hydroxychalcone butein was more than 6-fold that of metformin; negative control compounds increased uptake by no more than 25%. Dinitrophenol and four 4′-hydroxychalconoids also suppressed hepatocyte G6Pase as well as, or more effectively than metformin, whereas the unsubstituted parent compound chalcone, devoid of uncoupling activity, had no effect. Conclusions: Activities key to glycemic control can be induced by a wide range of weak uncouplers, including compounds free of difficult to metabolize groups typically associated with uncouplers. General significance: Uncoupling represents a valid and possibly more efficient alternative to inhibition for triggering cytoprotective effects of therapeutic relevance to insulin resistance in both muscle and liver. Identification of actives of natural origin and the insights into their structure-activity relationship reported herein may lead to alternatives to metformin.

Introduction: The antidiabetic drug metformin decreases hyperglycemia through insulin-like and insulin-potentiating effects in liver and skeletal muscle cells [1B-3B]. It also exerts an insulin-sensitizing effect in these tissues, presumably through a reduction of intracellular lipid accumulation [4B, 5B] brought about by increased oxidative capacity and altered fuel preference [6B, 7B]. These effects, both acute and chronic, can be attributed to the activation of the insulin-independent AMP-activated protein kinase (AMPK) signaling pathway [8B]. AMPK, extremely sensitive to AMP but inhibited by high concentrations of ATP, functions to monitor and protect energy homeostasis [9B, 10B]. In response to a perturbation of this homeostasis resulting from increased energy demand or decreased energy supply, it triggers acute corrective mechanisms that include the reduction of non-essential energy expenditure and the increase of energy production through the stimulation of substrate uptake and oxidation, as well as gene-expression-level effects for protecting homeostasis against future perturbations, including the increase of substrate uptake capacity (i.e., upregulation of transporter content) and of oxidative capacity (i.e., mitochondriogenesis). In light of its critical role in metabolic regulation, AMPK is considered a key therapeutic target for insulin resistance and associated metabolic diseases [11B-13B].

Metformin does not directly stimulate AMPK. Instead, it induces its activation by perturbing energy homeostasis [8B]. Specifically, it induces disruption of oxidative phosphorylation through inhibition of complex I of the electron transport chain [8B, 14B, 15B] thereby reducing the capacity for ATP resynthesis; if the maximal rate of ATP resynthesis becomes insufficient to meet cellular demand, then the total concentration of ATP must fall, thereby removing inhibition to AMPK activation. Accordingly, the main complication associated with this mechanism is systemic acidosis resulting from increased reliance on anaerobic metabolism (i.e., glycolysis). Indirect activation of AMPK can similarly be induced by other inhibitors of complex I [16B, 17B], as well as by inhibition of oxidative phosphorylation at various sites downstream of complex I [18B-20B]. New inhibition-based AMPK activators are currently undergoing clinical trials [21B]. Interestingly, the muscle and liver effects of thiazolidinedione insulin-sensitizers may be due to metformin-like inhibitory activity [22B], unrelated to their PPAR [symbol] agonist activity.

Alternatively, indirect activation of AMPK can be induced by uncoupling of oxidation from phosphorylation [16B], whereby protons pumped from the mitochondrial matrix into the mitochondrial intermembrane space by a normally-functioning electron transport chain are permitted to short-circuit ATP synthase and pass back across the mitochondrial inner membrane without transduction of their potential energy. This decrease in metabolic efficiency, like inhibition of oxidative phosphorylation, reduces the capacity for ATP resynthesis. However, in contrast to inhibition, uncoupling can be hypothesized to perturb energy homeostasis even in the absence of overt metabolic stress. Indeed, any increase in proton leakage across the mitochondrial inner membrane is compensated by increased flux through the electron transport so as to protect the proton motive force. As this increased flux and all the reactions that support it do not return increased ATP resynthesis, they effectively represent energy expenditure, indirectly consuming ATP. Such increase in ATP consumption can be expected to result in increased production of AMP by the adenylate kinase reaction, and from this, the activation of AMPK, whether or not uncoupling is of sufficient magnitude to impact the concentration of ATP. This potential distinction may be exploited to minimize risk of metabolic complications. Moreover, the distinction may be expected to translate into increased efficacy in skeletal muscle. Indeed, because the maximal oxidative capacity of muscle can be orders of magnitude greater than that necessary to meet resting energy demand, perturbing energy homeostasis through a reduction of capacity alone may be more difficult than in other cell types. Metformin's low efficacy in skeletal muscle relative to liver supports this notion [23B]. A second proposed advantage of uncoupling over inhibition is that the increased flux through the electron transport chain stimulated by uncoupling protects against the generation of free radicals associated with low flux and high mitochondrial membrane potential [24B, 25B], a condition which, along with the intracellular accumulation of lipids [26B-28B], may be causal to insulin resistance [25B, 29B]. Indeed, physiologically-regulated uncoupling mediated by members of the uncoupling protein family is an important mechanism for controlling oxidative stress [30B]. In sharp contrast, some forms of inhibition may contribute to free radical generation [31B]. Finally, whereas an inhibitor must interact directly with a target component of oxidative phosphorylation, compounds that induce uncoupling (i.e., uncouplers), in their simplest form, are small lipophilic compounds that cyclically shuttle protons across the inner mitochondrial membrane unaided and without interaction with integral membrane proteins [30B-33B]. Therefore, whereas inhibitory activity can be expected to require specific three-dimensional structure, Mitchellian uncoupling activity is not subject to key-in-lock type structural constraints, but is instead dependent only on an appropriate combination of acid-dissociation behavior and physicochemical properties conducive to transmembrane diffusion. This translates into an expansive chemical space and great flexibility for drug design and the optimization of safety independently of activity, for example, by selecting for highly active but easily-metabolized and short-lived (i.e., metabolically-unstable) compounds that are unlikely to cause sustained upregulation of glycolysis in the event of overt metabolic stress.

It is surprising that uncoupling has received little attention as a mechanism for the treatment and prevention of insulin resistance in light of such pontential advantages over inhibition of oxidative phopshorylation. One reason may be that uncouplers are best known as industrial insecticides, herbicides, and fungicides, and that the prevalence of naturally-occurring compounds with uncoupling activity but low persistence is underappreciated. Our interest in the therapeutic potential of uncouplers stems from our own observations that uncoupling-induced activation of AMPK is frequently the basis of the increase in glucose uptake induced by plant-based traditional treatments for diabetes [34B-36B] or by plant-derived compounds [37B], and that such products induce robust and sustained activation of AMPK signaling in spite of only mild and transient metabolic stress [34B, 35B, 37B]. The present work begins to address the hypotheses developed above while building on our recent findings. Specifically, the main purpose of this study was to demonstrate that weak uncouplers can be more efficacious than metformin for upregulating skeletal muscle cell glucose uptake, while being of comparably high efficacy for suppressing hepatocyte glucose output. This is demonstrated with uncouplers spanning a wide range of structure and physicochemical properties, including the reference weak uncoupler 2,4-dinitrophenol and more than a dozen naturally-occurring compounds of the type likely to be found in plant-based traditional treatments of diabetes. The latter, identified here by screening for uncoupling activity in isolated mitochondria, are all composed exclusively of C, H, and O, and therefore devoid of the notoriously difficult to metabolize chemical groups associated with reference uncouplers. The findings and observations reported herein have applications for the development of alternatives to metformin with greater skeletal muscle activity and greater potency without commensurate increase in incidence of metabolic complications.

Materials and Methods: Test compounds and reagents: Metformin (1,1 dimethylbiguanide hydrochloride), phenformin (phenethylbiguanide hydrochloride), 2,4-dinitrophenol, and fifty naturally-occurring phenolic compounds selected for screening (listed in Table 2 with Chemical Abstract Service (CAS) registry numbers) were obtained from Sigma-Aldrich (St-Louis, Mo.), with the exception of 4′-hydroxychalcone, homobutein, amentoflavone, cupressuflavone, and sciadopitysin obtained from Extrasynthèse (Genay-Cedex, France), and of datiscetin and pinosylvin obtained from Sequoia Research Products (Pangbourne, UK). Five compounds screened exist as two stereoisomers; a racemic mixture was used for the compounds naringenin, hesperetin, and silibinin; the (+) isomer was used for catechin; the (−) isomer was used for epigallocatechin gallate. Purity of all test compounds was ≧95%, with the exception of kaempferol (90%), frangulic acid (90%) and curcumin (94% curcuminoid content). Compounds were solubilized in dimethyl sulfoxide (DMSO) at a concentration of 100 mM, with the exception of metformin and anthraquinone, solubilized in water at 400 mM and 100 mM respectively. Compounds were aliquoted and stored at −20° C. in the dark. Aliquots were thawed immediately prior to use and subjected to a single freeze-thaw cycle. All other reagents were from Sigma-Aldrich, unless otherwise specified.

2.2 Screening of Compounds for Uncoupling Activity in Isolated Mitochondria

Isolation of mitochondria and measurement of oxygen consumption were performed as previously described [20B, 34B, 35B]. Mitochondria were isolated from the liver of male Wistar rats weighing 200-225 g. Animal care and handling conformed to the guidelines of the Canadian Council on Animal Care and of the Université de Montréal. Rats were anesthetized with sodium pentobarbital (50 mg/kg body weight). The portal vein was cannulated and the hepatic artery and infrahepatic inferior vena cava were ligated. The liver was flushed with 100 ml of Krebs-Henseleit buffer (25 mM NaHCO₃, 1.2 mM KH₂PO₄, pH 7.4, 250 mM NaCl, 4.8 mM KCl, 2.1 mM CaCl₂, 1.2 mM MgSO₄) at 22° C. and excised. Mitochondria were isolated from 2 g of tissue as per Johnson and Lardy [38B]. Briefly, tissue was homogenized on ice using a Teflon potter homogenizer in ice-cold Tris-sucrose buffer (10 mM Tris, pH 7.2, 250 mM sucrose, 1 mM EGTA) and centrifuged at 600×g for 10 min at 4° C. The supernatant was centrifuged at 15 000×g for 5 min at 4° C. The pellet was washed once in the same buffer, centrifuged at 15 000×g, washed once in EGTA-free Tris-sucrose buffer, and centrifuged again. The final pellet, containing viable mitochondria, was suspended in EGTA-free Tris-sucrose buffer and kept on ice. Protein content of the homogenate was determined by Lowry protein assay.

The effects of test compounds on rate of oxygen consumption of isolated mitochondria were assessed with a Clark-type oxygen microelectrode system with a 1 ml reaction chamber (Oxygraph; Hansatech Instruments; Norfolk, UK). One mg of mitochondrial protein was added to respiration buffer (5 mM KH₂PO₄, pH 7.2, 250 mM sucrose, 5 mM MgCl₂, 1 mM EGTA, and 2 μM of the complex I inhibitor rotenone) at 25° C. in the reaction chamber, for a final volume of 990 μl. State 4 respiration was initiated one minute later by the injection of 6 mM (final concentration) of the complex II substrate succinate, and the basal rate of oxygen consumption per mg mitochondrial protein was determined over the next 2 min. Test compound was then injected and its effect on the rate of basal oxygen consumption was assessed over at least 1 min. Oxidative phosphorylation (state 3 respiration) was then induced by the addition of 200 μM (final concentration) ADP and the ADP-stimulated rate of oxygen consumption per mg mitochondrial protein in the presence of compound was determined. Multiple runs of the vehicle-(DMSO) control were conducted at the beginning and end of each experimental session in order to establish session-normal basal and ADP-stimulated rates of oxygen consumption, and to ensure no loss in mitochondrial viability over the duration of the session, typically less than 4 h from the end of the isolation protocol. Preparations consistently yielded a coupling ratio (ADP-stimulated rate of oxygen consumption/basal rate of oxygen consumption) of 4.5 to 5. Compounds were all screened at 100 μM in 0.1% DMSO in two to three different mitochondrial preparations. Compounds that measurably stimulated basal rate of oxygen consumption at this concentration were also tested at 25 μM. The effect of each compound was evaluated as: 1) increase in basal rate of oxygen consumption per mg protein, a direct measure of the magnitude of uncoupling effect; 2) decrease in functional capacity per mg protein, a measure of the magnitude of the uncoupling effect plus any concomitant inhibitory effect, where functional capacity is defined as the difference of the ADP-stimulated rate of oxygen consumption (considered the maximal functional rate of oxygen consumption) and basal rate of oxygen consumption (considered the rate of oxygen consumption driven by proton leak and that does not contribute to ATP resynthesis). This assumes that the rate of proton leak is independent of flux through oxidative phosphorylation. Calculations were as follows: the average functional capacity of the vehicle control experiments for a given session was calculated by subtracting the session-average basal oxygen consumption from the session-average ADP-stimulated oxygen consumption. For 1) above, the absolute increase in basal oxygen consumption measured in a given experiment was expressed as a percentage of the session-average vehicle control functional capacity. By this definition, complete uncoupling 100%) was said to have occurred if basal oxygen consumption equaled or surpassed ADP-stimulated oxygen consumption, effectively abolishing capacity for ATP synthesis. For 2) above, the functional capacity measured in a given experiment was expressed as a percentage of the session-average vehicle control functional capacity to give the residual functional capacity. Finally, the contribution of inhibitory activity, if any, to diminished functional capacity was estimated by subtracting the decrease in functional capacity attributable to uncoupling from the total decrease in functional capacity.

In addition, dose-escalation experiments were performed with 2,4-dinitrophenol and the fourteen compounds that exhibited the greatest uncoupling activity at 100 μM in order to determine the concentration at which 50% uncoupling is induced (U₅₀) and to assess the concentration-activity relationship; test compound was injected repeatedly over the course of a single experiment and the cumulative effect on basal rate of oxygen consumption was assessed after each injection. DMSO was confirmed to have no effect on basal oxygen consumption at a concentration of up to 2% under this paradigm.

2.3 ³H-deoxyglucose uptake in C2C12 myotubes: Cell culture and the ³H-deoxyglucose uptake assay were performed as previously described [34B, 35B, 37B]. Briefly, C2C12 murine skeletal myoblasts (American Type Culture Collection; Manassas, Va.) were cultured under standard conditions in 12-well plates. Cells were proliferated to 80% confluence in high-glucose Dulbecco's modified Eagle's medium (DMEM; Wisent; St-Bruno, QC) supplemented with 10% fetal bovine serum (FBS; Wisent), 10% horse serum (HS), and antibiotics. Differentiation into multinucleated myotubes was then promoted with DMEM supplemented with 2% HS and antibiotics. All uptake assays were performed on 7-day differentiated cells, and treatments were timed accordingly. Cells were treated with metformin (100 or 400 μM), phenformin (100 μM), 2,4-dinitrophenol and other test compounds (25, 50 or 100 μM), or vehicle (DMSO) in complete differentiation medium. DMSO concentration was fixed at 0.1% for all conditions. Cells were routinely inspected for abnormal morphology by phase-contrast microscopy at the conclusion of the treatment period. Thirty minutes prior to uptake experiments, cells were equilibrated in Krebs-phosphate buffer (KPB; 20 mM HEPES, 4.05 mM Na₂HPO₄, 0.95 mM NaH₂PO₄, pH 7.4, 120 mM NaCl, 5 mM glucose, 4.7 mM KCl, 1 mM CaCl₂ and 1 mM MgSO₄) at 37° C. Insulin, prepared freshly, was added to some vehicle control wells at 100 nM during this period. Cells were then washed twice in glucose-free KPB at 37° C. before incubation for exactly 10 min at 37° C. in glucose-free KPB containing 0.5 μCi/ml 2-deoxy-D-[1-³H]glucose (Amersham Biosciences; Buckinghamshire, UK). Cells were then placed on ice and immediately washed three times with ice-cold KPB. Cells were inspected for monolayer detachment and lysed in 0.1 N NaOH with scraping. Lysates were transferred to Ready-Gel scintillation fluid (Beckman Coulter, Inc.; Fullerton, Calif.) and incorporated radioactivity was assessed in a liquid scintillation counter (1219 RackBeta; Perkin-Elmer; Waltham, Mass.). Three independent experiments of 18 h treatment duration were performed for each of the selected test compounds, with three replicates per condition per experiment. Vehicle-control and 50 μM 2,4-dinitrophenol conditions were included on every plate. Some compounds were also tested at 50 μM under the following conditions: a 15 h treatment with vehicle only followed by a 3 h treatment with the test compound; a 15 h treatment with the test compound followed by a 3 h repeat treatment.

2.4 Glucose-6-phosphatase activity in H4IIE hepatocytes: H4IIE murine hepatocytes (American Type Culture Collection) were cultured to confluence in 12-well plates in DMEM supplemented with 10% FBS and antibiotics. Cells were treated with insulin (100 nM; prepared freshly), metformin (400 μM), 2,4-dinitrophenol or other selected test compounds (12.5-25 μM), or vehicle (DMSO) for 16 h in serum-free medium. Effects of test compounds on cellular viability were assessed by measuring the release of lactate dehydrogenase (LDH) into the culture medium at the end of a 16 h treatment using a commercial kit (Cytotoxicity Detection Kit; Roche Diagnostics; Laval, QC) as per the manufacturer's instructions; LDH release was expressed as % of total (medium+lysate) LDH content for each well. Cells were also routinely inspected for abnormal morphology by phase-contrast microscopy at the conclusion of the treatment period. Following treatment, cells were washed in HEPES-buffered saline (10 mM HEPES, pH 7.4, 150 mM NaCl) at 37° C. Glucose-6-phosphatase (G6Pase) activity was assessed by measuring the rate of glucose formation in the presence of a non-limiting amount of glucose-6-phosphate (G6P), as done by others [39B, 40B]. Glucose production was measured with a commercial glucose assay kit (AutoKit Glucose; Wako Diagnostics; Richmond, Va.). Two hundred μl of AutoKit Glucose buffer solution diluted 1:4 in water were added to each well. Cells were then lysed by the addition of 50 μl of 0.05% Triton X-100 in similarly diluted AutoKit Glucose buffer solution. Immediately following addition of Triton X-100, 20 mM (final concentration) of G6P were added to each well for a final volume of 2754 Plates were then incubated for exactly 40 min. At 37° C., after which time 500 μl of AutoKit Glucose color reagent were added and incubation was continued for exactly 5 min. Samples were then rapidly transferred to microcentrifuge tubes. Fifty μl were removed for assay of total protein content in order to account for effects of test compounds on cellular viability or proliferation [41B]. A commercial protein assay kit based on the Bradford method was used (Protein Assay; Bio-Rad Laboratories; Hercules, Calif.). This assay was observed to be unaffected by high concentrations of phenolic compounds. The remaining volume was centrifuged at 3000×g for 5 min. Absorbance of the supernatant was measured at 505 nm at ambient temperature and glucose concentration was calculated from a standard curve performed in parallel. Control wells without exogenous G6P were included on each plate for each treatment condition and activity measured from these wells was subtracted from activity measured in the presence of exogenous G6P. G6Pase activity calculated in this way was then expressed normalized to protein content on a well-by-well basis. Two independent experiments in cells of different passages were performed for each of the selected test compounds, with four to six replicates per condition per experiment.

2.5 Calculation of Physicochemical Properties of Screened Compounds:

The octanol-water partition coefficient (P_{octanol-water}), an estimate of a compound's lipophilicity, and the acid-dissociation constant (pKa) of each ionizable group were predicted using the Marvin 5.2 chemoinformatics suite (academic package; ChemAxon Kft.; Budapest, Hungary) from manually-drawn structures validated against structures provided by the compound manufacturers and against public database entries. The Protonation calculator plug-in was used to estimate pKa at a temperature of 37° C. The Partitioning calculator plug-in was used to estimate P of the neutral molecular species under default ionic strength conditions. Calculated log P_{octanol-water} and pKa were verified against published experimental values whenever available. Molecular geometry was assessed from the three-dimensional rendering of the low-energy conformer of each compound in MarvinView.

2.6 Statistical Analysis:

Results are reported as mean±SEM, with the number of replicates and number of independent experiments indicated. Data were analyzed by one-way analysis of variance with a Fisher post-hoc test and statistical significance set at p 0.05.

3. Results and Discussion

3.1 The Reference Weak Uncoupler 2,4-Dinitrophenol as Model Compound

3.1.1 Enhancement of Glucose Uptake in C2C12 Myotubes:

The hypothesis that uncouplers of oxidative phosphorylation are generally more conducive to the enhancement of skeletal muscle glucose uptake than compounds that perturb energy homeostasis through other forms of disruption of oxidative phosphorylation, including the antidiabetic drug metformin, was addressed first by comparing the effects of the well-studied uncoupler 2,4-dinitrophenol directly against those of metformin and of its more potent derivative phenformin in differentiated C2C12 myotubes. Specifically, effects on basal (non-insulin-stimulated or constitutive) uptake of labeled deoxyglucose were assessed following an 18 h treatment with the test compounds. Glucose utilization by skeletal muscle is key to glycemic control and glucose uptake is rate-limiting to this process [42B, 43B]. Uptake is stimulated acutely by insulin [44B] and by AMPK activation [45B]. AMPK can also chronically increase constitutive uptake and insulin stimulated uptake through an increase in the expression of glucose transporters, and hence of uptake capacity [46B, 47B]. An18 h treatment duration was selected to favor upregulation of glucose uptake capacity rather than acute translocation and/or activation of pre-existing glucose transporters as the mechanism of enhancement of glucose uptake [48B].

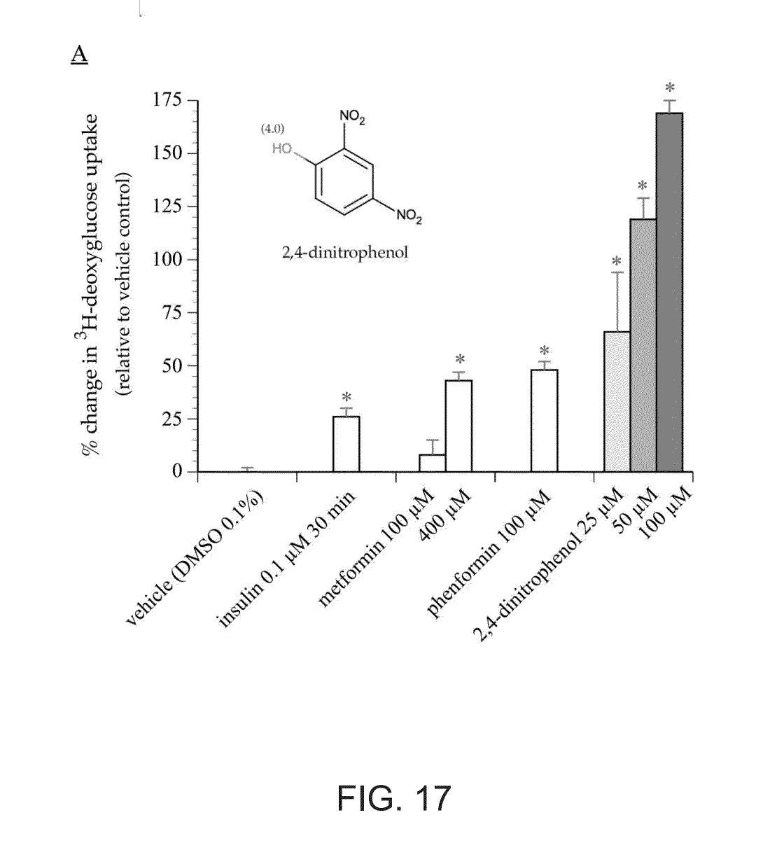

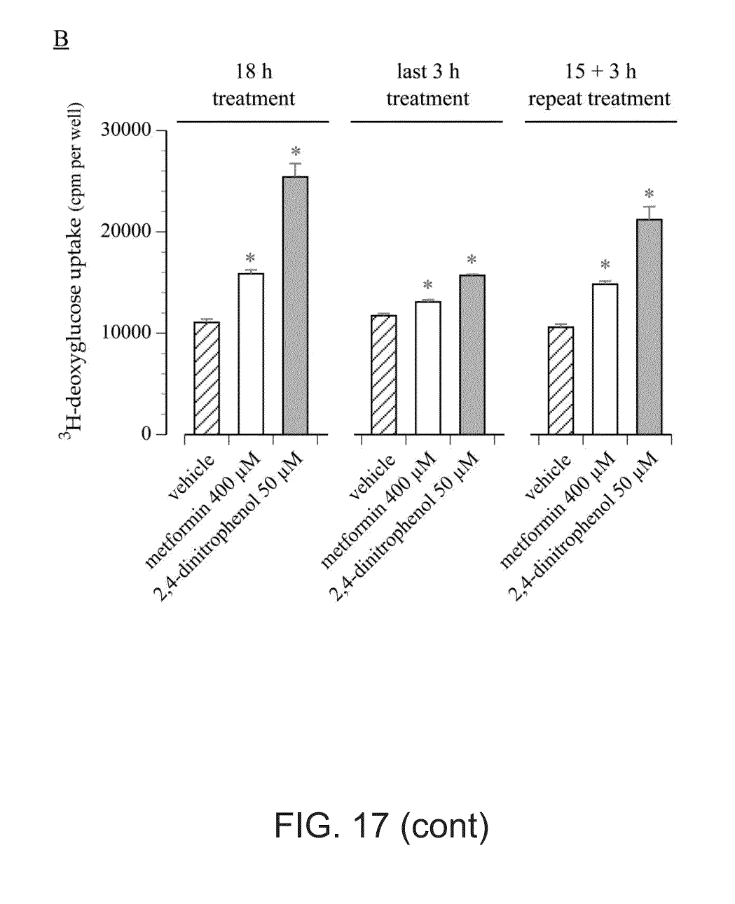

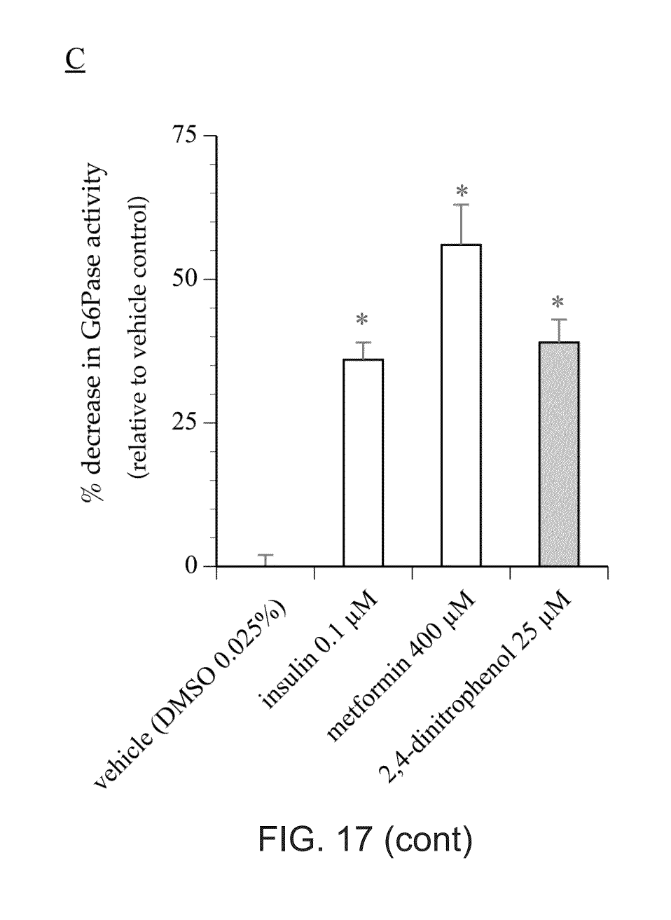

Under this paradigm, 2,4-dinitrophenol increased uptake by 66, 119 and 169% at 25, 50 and 100 μM, respectively, whereas metformin (400 μM) and phenformin (100 μM) increased basal uptake by 43% and 48%, respectively (FIG. 17A). For reference purposes, a supraphysiological dose of insulin (100 μM) administered to vehicle-treated cells 30 min prior to the deoxyglucose assay stimulated uptake by 26%. The importance of treatment duration to the observed effects of 2,4-dinitrophenol as well as those of metformin was further explored by comparing the results of an 18 h treatment to those of a 3 h treatment in parallel cells pre-treated 15 h with vehicle, and those of a 15 h treatment in parallel cells followed by a 3 h repeat treatment. Both 2,4-dinitrophenol at 50 μM and metformin at 400 μM induced a greater increase in glucose uptake under the original 18 h treatment paradigm than under the other two paradigms (FIG. 17B).

If the insulin-stimulated rate of uptake is taken as an approximation of the maximal uptake capacity of untreated cells, then sustained increases in rate of basal glucose uptake significantly greater than can be stimulated acutely by insulin, as induced here by treatment with 2,4-dinitrophenol or biguanides, support the notion of an expression-level upregulation of the capacity for uptake; while not tested, it can be expected that the effect of acute insulin stimulation in treated cells would be proportional to that in untreated cells. 2,4-dinitrophenol has been shown by other groups to induce acute insulin-like increases of glucose uptake in cultured muscle cells through the translocation of GLUT1 and GLUT4 transporters [49B, 50B] and increases of glucose uptake in skeletal muscle ex-vivo mediated by AMPK [16B]. While the more important effects of a longer duration treatment with 2,4-dinitrophenol have not been previously been addressed, the contrast between acute effects involving transporter translocation and activation and longer-term effects of greater magnitude involving increased transporter mRNA and protein content, has been well described for the complex IV inhibitor azide [48B, 51B].

The observations that several hours are needed for the full expression of the effect of 2,4-dinitrophenol and metformin, and that, following the three hour treatment, effects are only on the order of those reported above for a 30 min stimulation with insulin, are again consistent with an enhancement of glucose uptake capacity through increased expression of effector proteins. That overall upregulation was lower under the re-treatment paradigm than under the 18 h treatment paradigm may reflect a transient inhibition of translation and other non-essential energy-consuming processes resulting from a reactivation of AMPK; while not performed, assessment of uptake 18 h rather than 3 h following the repeat treatment would be expected to show an increase in capacity greater than induced by the single treatment paradigm.

3.1.2 Suppression of glucose-6-phophatase in H4IIE hepatocytes:

The hypothesis that uncouplers of oxidative phosphorylation can generally suppress liver glucose output as effectively as agents that perturb energy homeostasis through other forms of disruption of oxidative phosphorylation, including metformin, was addressed first by comparing the effects of 2,4-dinitrophenol directly against those of metformin in H4IIE hepatocytes. Specifically, suppression of G6Pase activity was assessed following a 16 h treatment with the test compounds. Hepatic glucose release is key to glycemic control and G6Pase activity is a rate-limiting step of this process [52B, 53B]. Expression of the catalytic subunit of hepatocyte G6Pase is negatively regulated by insulin [54B, 55B] as well as by AMPK [56B-58B].

Under this paradigm, 2,4-dinitrophenol at 25 μM significantly suppressed G6Pase activity per mg total protein by 39% and metformin at 400 μM suppressed activity by 56% (FIG. 17C). For reference purposes, a 16 h treatment with 100 nM insulin suppressed G6Pase activity by 36%, whereas a 16 h treatment with 1 μM of the glucocorticoid dexamethasone increased G6Pase activity by 56%. 2,4-dinitrophenol was well-tolerated by H4IIE hepatocytes at the concentration tested, as supported by morphological assessment and by a rate of release of lactate dehydrogenase over the 16 h treatment comparable to that of vehicle-treated cells (results not shown). This insulin-like activity of metformin is well-known and accounts for much of this drug's anti-hyperglycemic activity [3B, 59B, 60B]. However, such insulin-like activity has not previously been attributed to 2,4-dinitrophenol or other uncouplers of oxidative phosphorylation.

It should be noted that because the G6Pase catalytic subunit is a short-lived protein, an assay of the downregulation of G6Pase activity or of glucose output necessarily affords only limited resolution. It is therefore a possibility that by focusing on a different endpoint, such as the upregulation of a marker of oxidative capacity following several days of treatment, 2,4-dinitrophenol could be shown to be more efficacious than metformin in hepatocytes as in muscle cells, above. Alternatively, given the low oxidative capacity of hepatocytes as compared to skeletal muscle cells, including the C2C12 cell model in which oxidative capacity increases several fold during differentiation [61B, 62B], it may be difficult to subtly perturb energy homeostasis without inducing metabolic stress, as proposed to be possible in skeletal muscle with uncouplers.

3.2 Screening of Compounds for Uncoupling Activity in Isolated Mitochondria:

In order to more generally support results obtained with the reference uncoupler 2,4-dinitrophenol, above, a screening study for uncoupling activity in isolated mitochondria was performed so as to identify a wide sample of weak uncouplers of oxidative phosphorylation and an equally wide sample of closely related compounds with little to no uncoupling activity for testing in skeletal muscle cells and hepatocytes. In light of our previous observations of AMPK-mediated enhancement of glucose uptake by plant products with uncoupling activity [34B-37B], and given that uncoupling activity is associated with phenolic compounds of moderate to high lipophilicity that are ionizable at physiological pH [30B-33B], this screening focused on flavonoids and related plant metabolites.

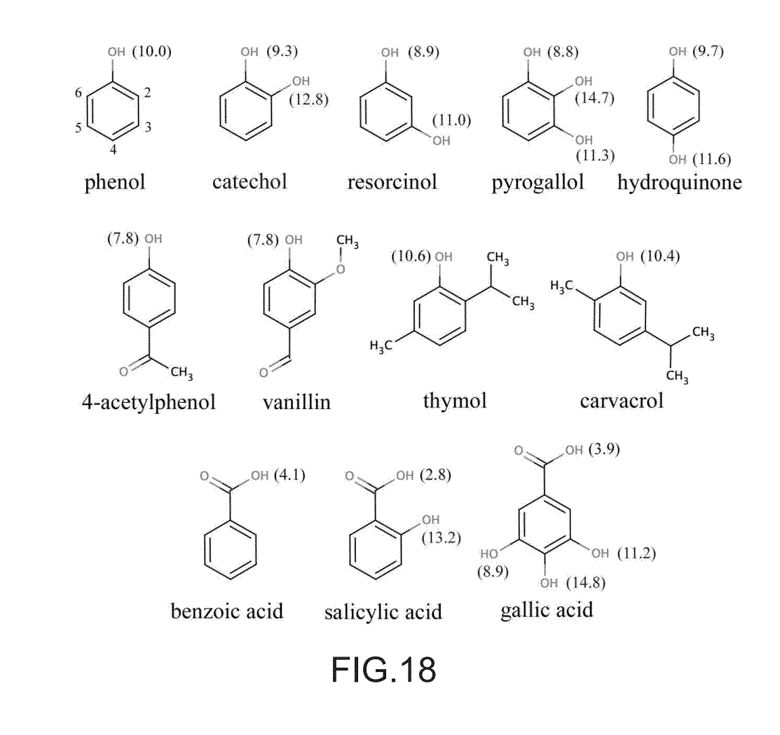

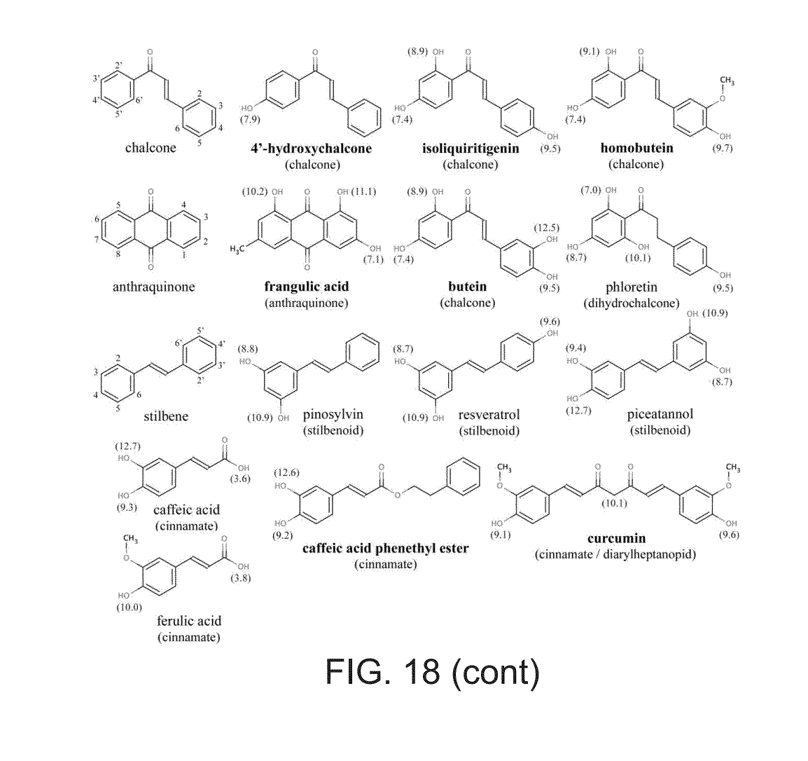

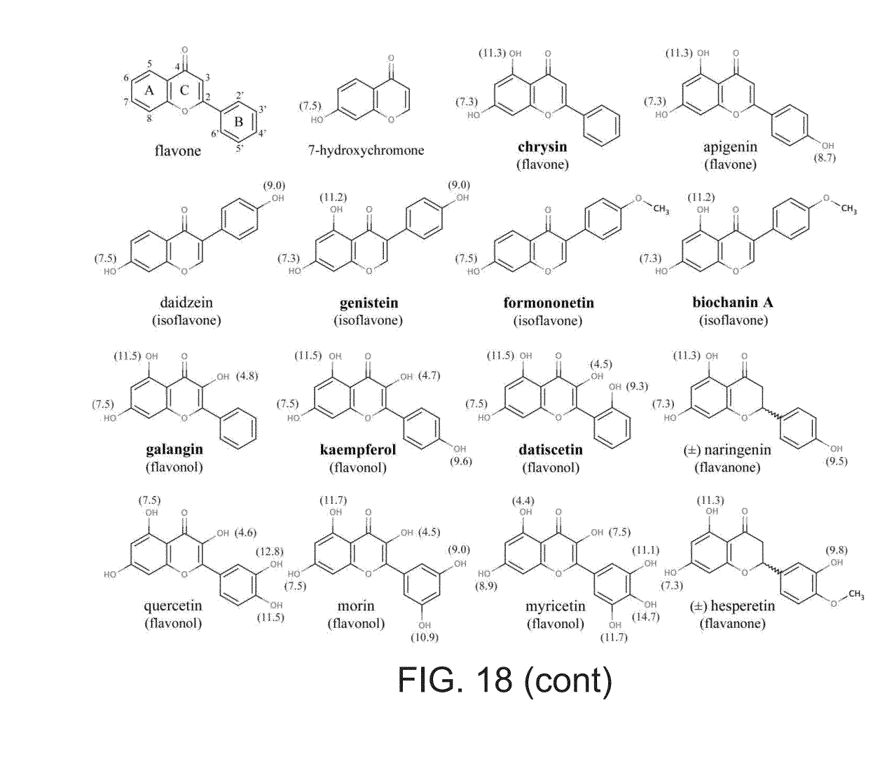

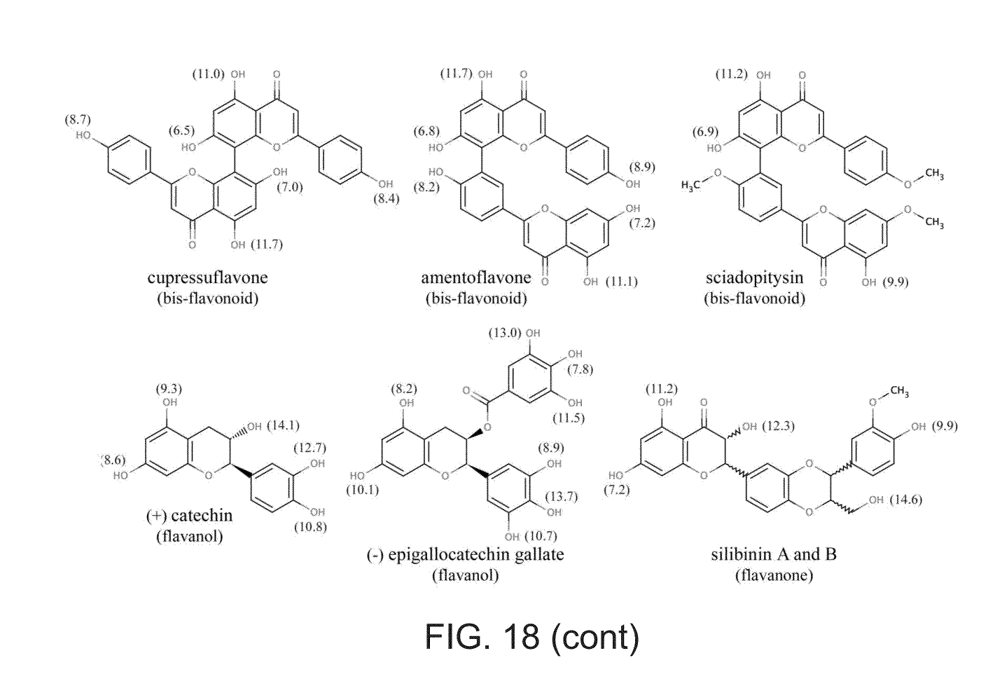

A total of fifty compounds were selected for screening from several phytochemical classes, including flavonoids (six different subclasses of these are represented), chalconoids, stilbenoids, anthraquinones, cinnamates, and simple naturally-occurring phenolics. The unsubstituted parent compound for each of the polycyclic classes, nonionizable at physiological pH and therefore presumably devoid of uncoupling activity, were included as negative controls for the screening study and subsequent cell-based assays, below. 2,4-dinitrophenol served as positive control. All compounds are listed in Table 2, grouped by phytochemical family and identified by traditional phytochemical name and by CAS registry number. Chemical structures are provided in FIG. 18. Selected compounds spanned a wide range of structure and physicochemical properties. However, all compounds considered were composed exclusively of C, H, and O.

Screening was performed by oxygraphy in isolated rat liver mitochondria. Specifically, compounds were tested at 100 μM for stimulation of the rate of basal (state 4 respiration) oxygen consumption (i.e., Increase in respiration not accompanied by a commensurate increase in resynthesis of ATP). This measure of uncoupling captures the activity of both classical proton shuttle uncouplers and of protein-assisted uncouplers, as well as that of cationophores or inducers of a cation conductance [30B, 31B]. Uncoupling was reported as a percentage of the rate of ADP-stimulated consumption (state 3 respiration) measured in vehicle-treated control mitochondria, thereby allowing the pooling of data obtained from different preparations; inter-preparation variability in coupling ratio and in rate of basal O₂ consumption ranged from 4.0 to 5.4 and from 8 to 21 nmoles per min per mg mitochondrial protein, respectively. At concentrations tested, none of the test compounds save the positive control were expected to induce uncoupling of sufficiently large magnitude to collapse the mitochondrial membrane potential; membrane potential was therefore not monitored.

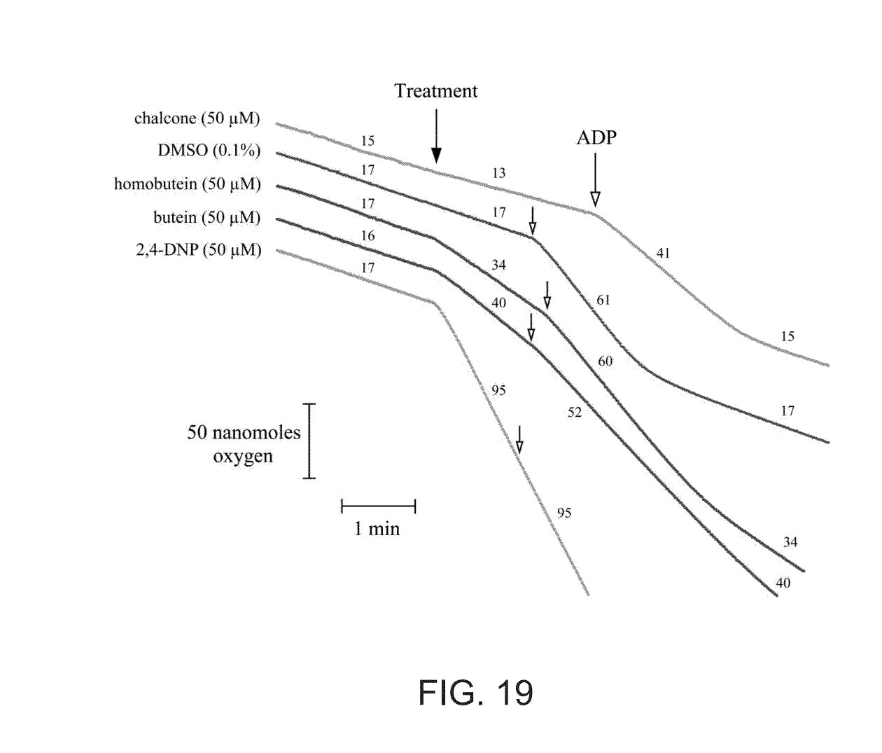

Fourteen compounds were found to induce more than 20% uncoupling (i.e., Increase in rate of basal oxygen consumption of at least 1.7- to 2-fold, depending on coupling ratio) at 100 μM (Table 2). These included seven flavonoids, four chalconoids, two cinnamates, and one anthraquinone. Representative tracings of oxygen consumption are shown in FIG. 19. Of these fourteen compounds, three induced complete uncoupling (i.e., rate of basal oxygen consumption≧control rate of ADP-stimulated oxygen consumption; increase in rate of basal oxygen consumption≧4.4- to 5.6-fold) at 100 μM: the chalconoid isoliquiritigenin, the flavone chrysin, and the isoflavone biochanin A. In addition to these fourteen compounds of particular interest, ten others induced 5 to 20% uncoupling at 100 μM while the balance of compounds exhibited little (seven compounds) to no activity (nineteen compounds). As expected, the non-ionizable parent compounds anthraquinone, chalcone, flavone, and stilbene figured among the inactive. Compounds exhibiting significant activity at 100 μM were further screened at 25 μM. At this concentration, eight induced more than 20% uncoupling (Table 2), including two capable of complete uncoupling: chrysin and the flavonol galangin.

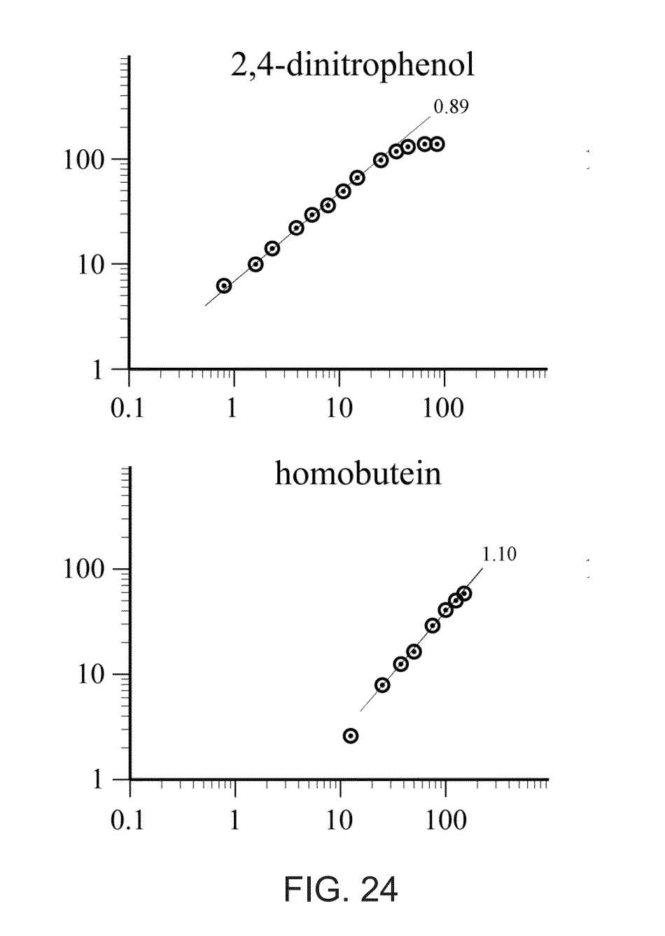

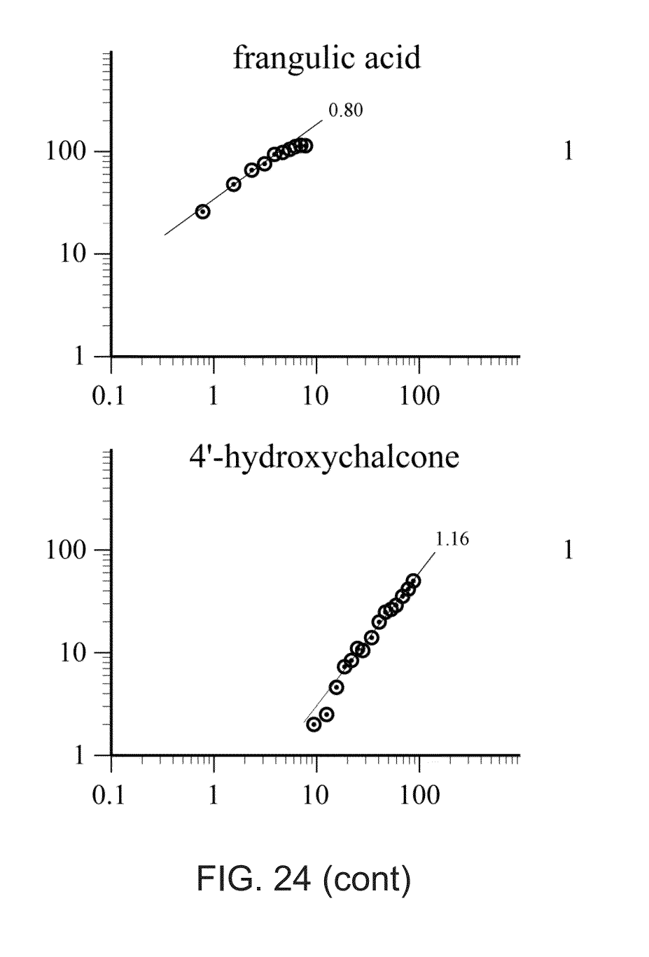

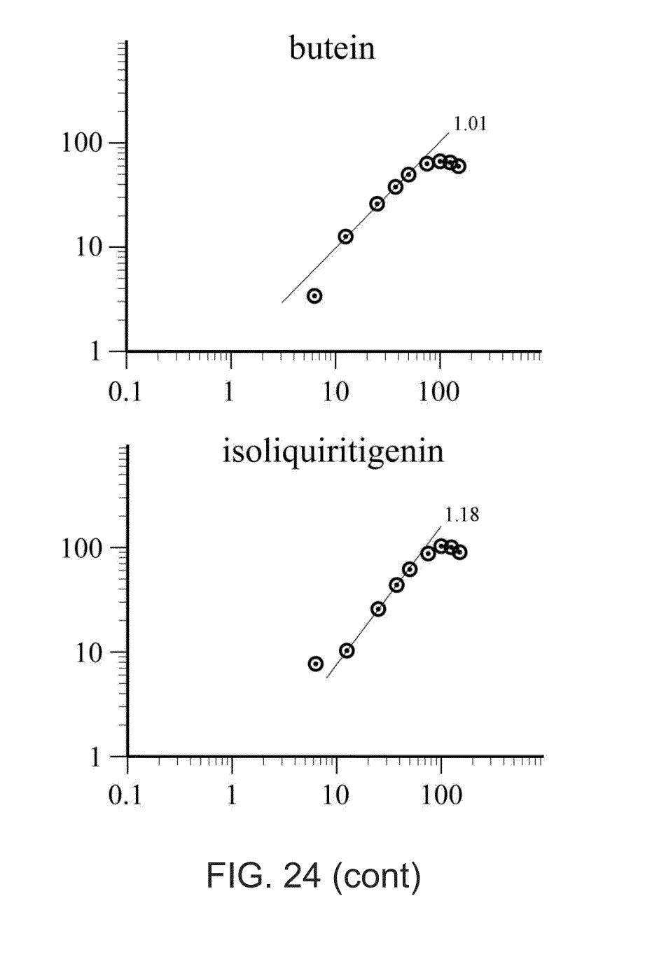

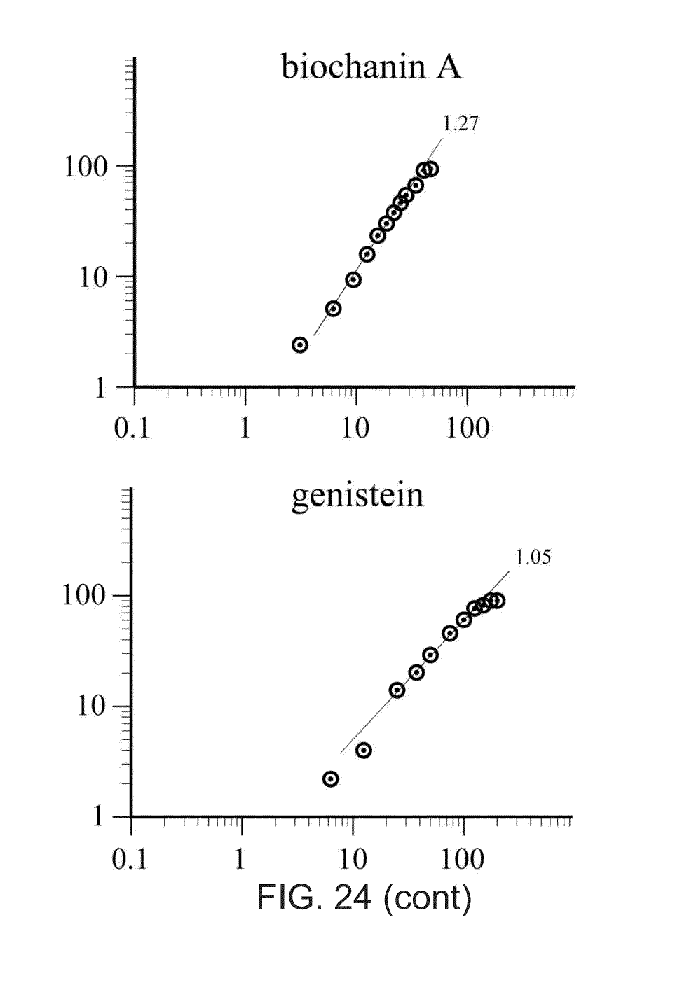

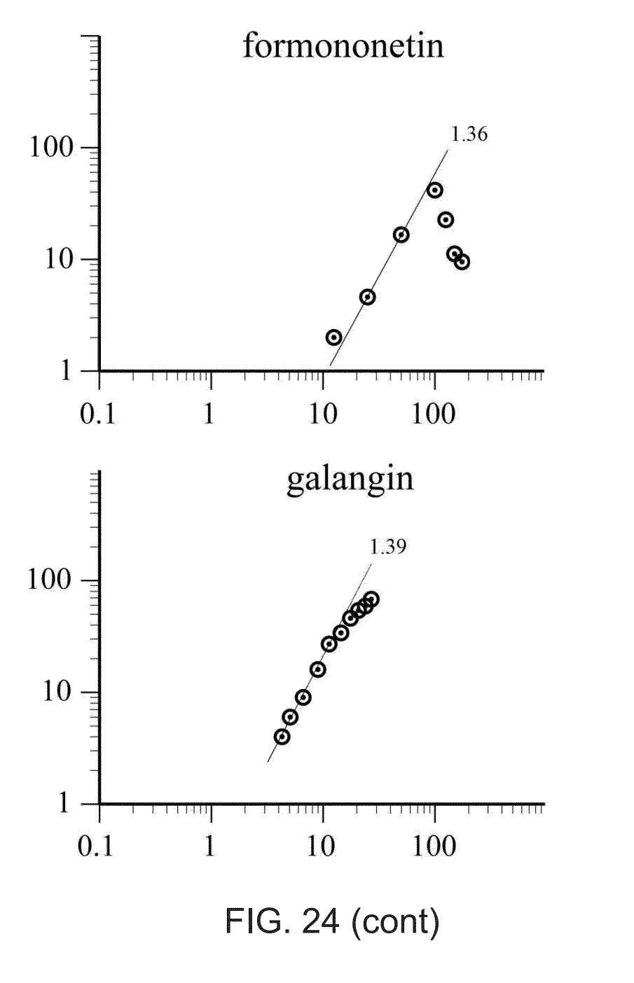

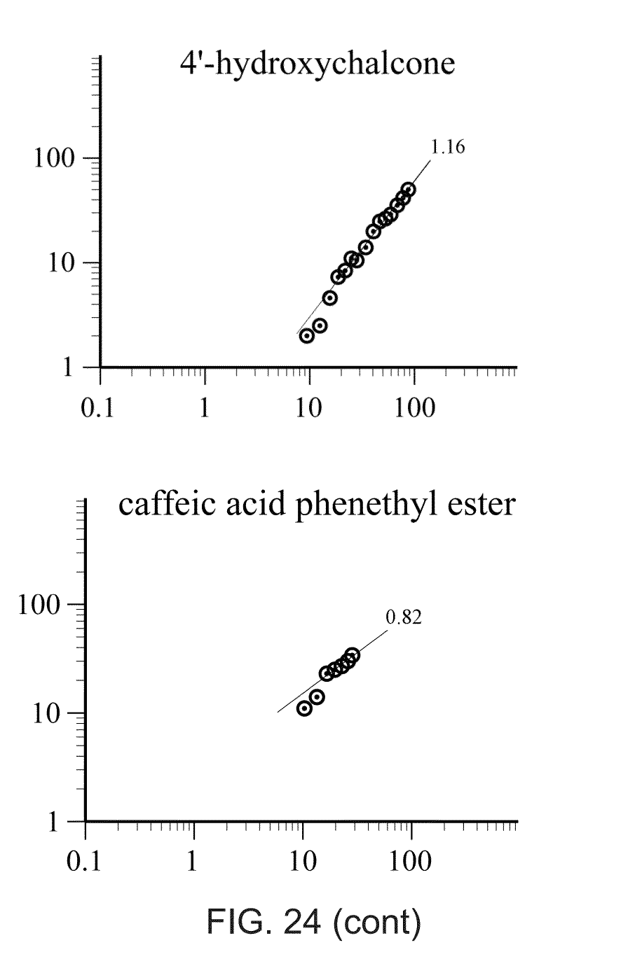

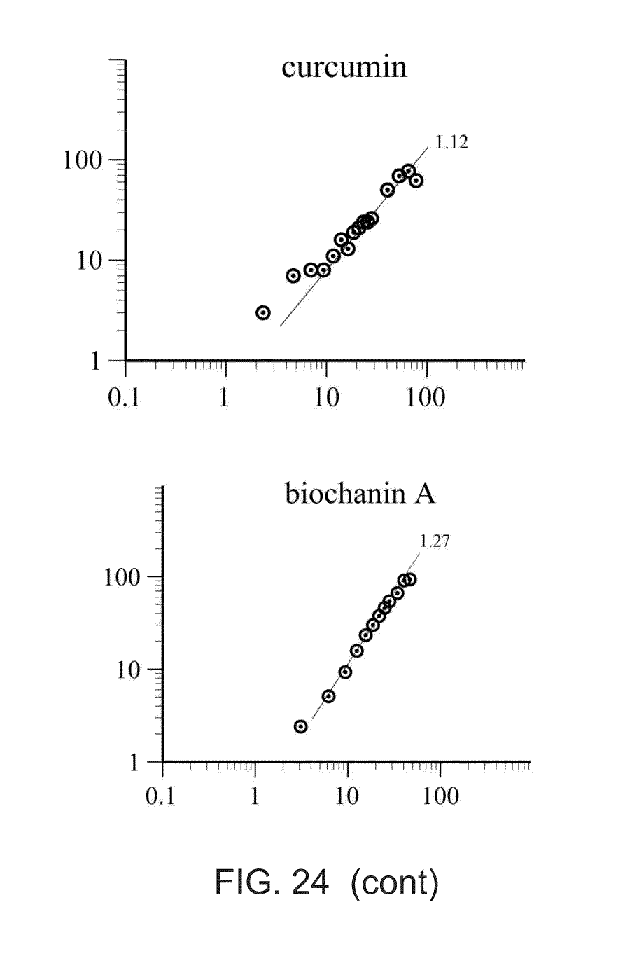

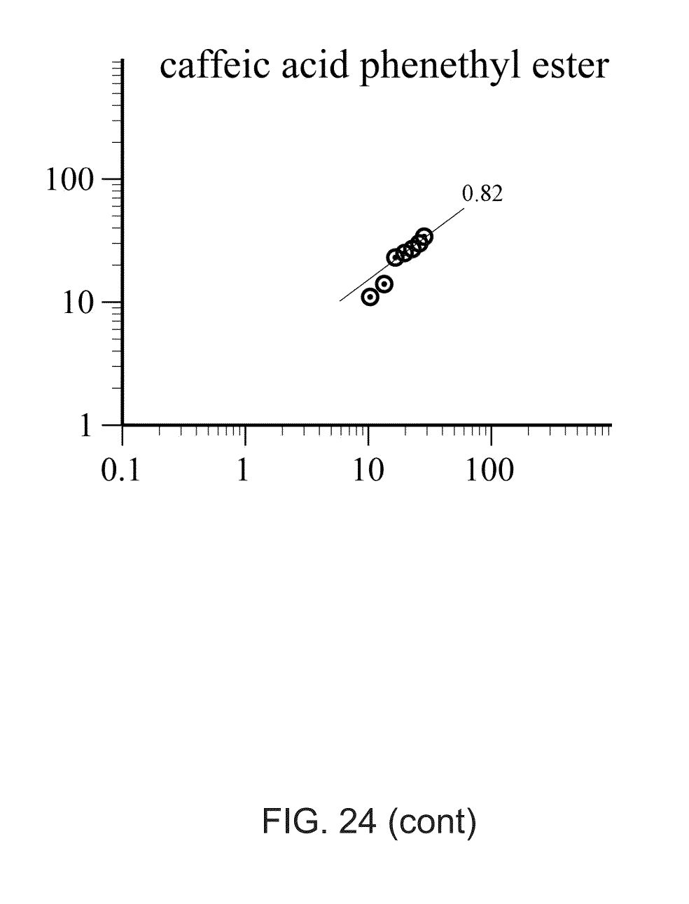

Additionally, the concentration-to-activity relationship of the fourteen most active and of 2,4-dinitrophenol was assessed using a dose-escalation approach. Results of representative experiments are plotted in FIG. 24. Concentration at which 50% uncoupling is induced (U₅₀), maximal inducible uncoupling, and slope of the linear portion of the relationship are reported in Table 2. The fourteen compounds of interest were found to span a considerable range of U₅₀, from 2 to 125 μM. It is noteworthy that four of these, the anthraquinone frangulic acid, the flavonols datiscetin and galangin, and the flavone chrysin, exhibited potency on the order of that of 2,4-dinitrophenol. Unexpectedly, while U₅₀ was generally reflective of activity measured at 100 μM, several compounds exhibited saturation well below maximal uncoupling (defined as stimulation in the rate of basal oxygen consumption induced by 2,4-dinitrophenol at 100 μM), in some cases even resulting in a peak-type concentration-activity relationship (FIG. 24). Because of such a relationship, the maximal activity assessed by dose-escalation was in four instances significantly greater than the uncoupling activity assessed at 100 μM; case in point, dose-escalation allowed for the identification of the anthraquinone frangulic acid as a fifth compound capable of complete uncoupling at 100 μM or below (Table 2).

A saturation effect at submaximal activity is indicative of inhibition of oxidative phosphorylation concurrent with uncoupling activity. Given that respiration was supported by the complex II substrate succinate, such inhibition may have occurred at any point along the electron transport chain downstream of complex I. Inhibition of substrate import may also have caused this effect [63B-65B].

As is characteristic of most uncouplers, all fourteen compounds exhibited submaximal activity over a narrow concentration range of less than two orders of magnitude and exhibited linearity over a portion of their concentration-to-activity relationship (FIG. 24). However, the slope of this linear portion varied greatly among compounds, perhaps indicating mechanistic differences. Indeed, classical proton shuttle uncouplers can be expected to exhibit a near 1:1 relationship between concentration and activity. A shallower, more extended relationship suggests facilitation of Mitchellian uncoupling through interaction with a protein constituent of the mitochondrial inner membrane, such as the adenine nucleotide translocase [30B, 31B, 66B]. Such a mechanism is thought to contribute in part to the activity of 2,4-dinitrophenol [30B], observed here to exhibit a slope of 0.89. Four compounds exhibited a slope markedly less than unity, the most notable of which was the flavonol datiscetin (slope of 0.44). A slope greater than unity may indicate augmentation of protonophoric activity through a cationophoric conductance. Several compounds exhibited a slope markedly greater than unity, the most notable of which were the flavonol galangin (slope of 1.39) and the isoflavone formononetin (slope of 1.36). Additional testing in the presence of inhibitors including carboxyatractylate, 6-ketocholestanol, and cyclosporin A, may provide further insight into the contribution, if any, of non-Mitchellian mechanisms.

Several compounds of the testset have been reported to exhibit uncoupling activity by others [67B-74B] or elsewhere [35B, 37B], as annotated in Table 2. It should be noted that in a number of cases, this activity has been identified in plant rather than animal mitochondria and reported potency is lower than that observed here; this may be attributed to known differences between these systems [75B]. Furthermore, several compounds of the testset have been proposed to be inducers of mitochondrial permeability transition [76B-78B]; if permeability transition can be induced immediately upon compound injection, then in some cases cationophoric activity may have contributed to the measured uncoupling activity.

Following assessment of stimulation of the rate of basal oxygen consumption, ADP was injected to induce oxidative phosphorylation (state 3 respiration) and the rate of ADP-stimulated oxygen consumption was then determined and compared to that measured under vehicle-control conditions in the same experimental session. While unrelated to the identification of compounds with uncoupling activity, the rate of ADP-stimulated oxygen consumption was observed to be inhibited by the majority of compounds tested (Table 2). Among the fourteen compounds with significant uncoupling activity, all but biochanin A and 4′-hydroxychalcone exhibited a reduction in functional capacity that was greater than could be expected from their uncoupling activity alone. In cases where a compound exhibited inhibitory activity in the absence of any significant uncoupling activity, as was notably the case for piceatannol, quercetin, silibinin, datiscetin, chalcone, resveratrol, morin, and amentoflavone, ATP synthase (i.e., complex V) could be concluded to be the site of inhibition. In cases of concurrent uncoupling and inhibition, the site of inhibition could not be determined by the methodology used here and could only be concluded to be downstream of complex I. However, unless the relationship between concentration and uncoupling activity exhibited submaximal saturation (discussed above), inhibition of ATP synthase could be suspected in these cases as well in light of the prevalence of such inhibitory activity among the types of polycyclic phytochemicals considered here; as annotated in Table 2, eleven compounds of the testset have been reported by others [73B, 79B-81B] or elsewhere [20B] to inhibit ATP synthase, whereas four have been reported to have other inhibitory effects [82B-84B]. It should be noted that in the event of concurrent uncoupling and inhibition downstream of complex I but upstream of ATP synthase, uncoupling activity as measured here would tend to be underestimated; this may be the case for butein, reported by others to be an inhibitor of complex II [83B]. Inhibitory activities were not further investigated.

3.3 Enhancement of Glucose Uptake in C2C12 Myotubes by Identified Uncouplers:

Fourteen naturally-occurring compounds found to induce more than 20% uncoupling at 100 μM in isolated rat liver mitochondria (Section 3.2) were tested for enhancement of basal glucose uptake in C2C12 myotubes following an 18 h treatment at 25 to 100 μM, as reported for the reference uncoupler 2,4-dinitrophenol, above (Section 3.1.1). To reinforce the hypothesized link between uncoupling activity and upregulation of glucose uptake, fourteen related compounds exhibiting little or no uncoupling activity at 100 μM in isolated mitochondria were also tested under the same conditions.

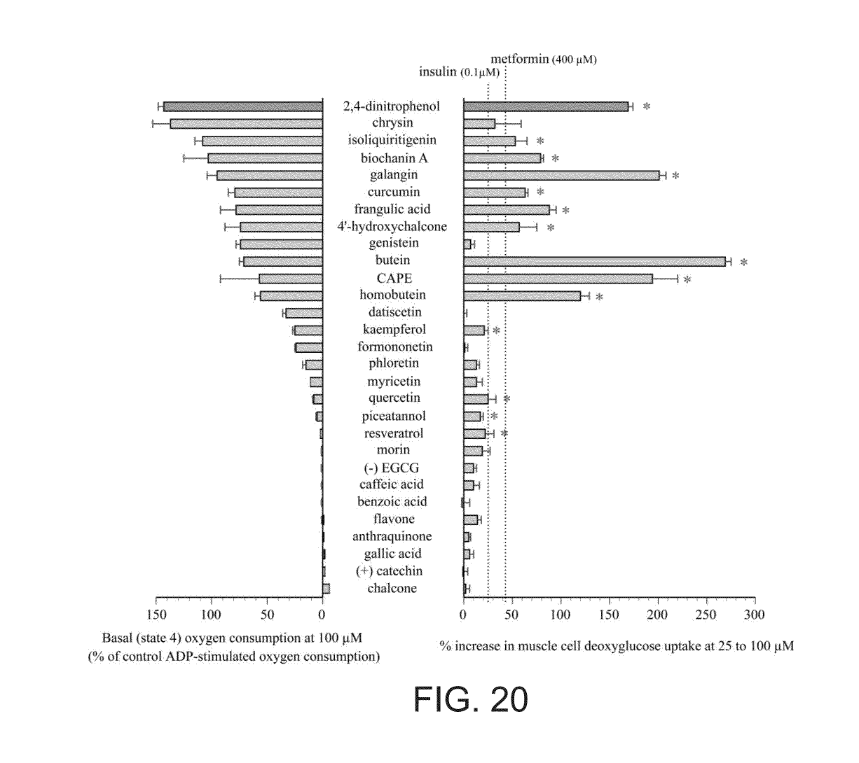

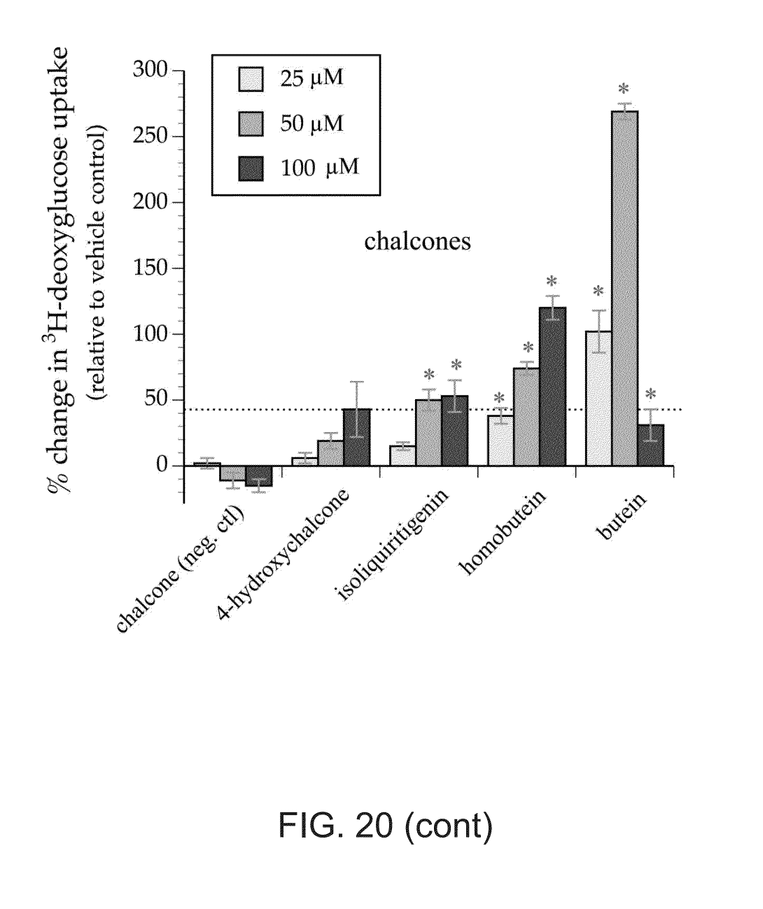

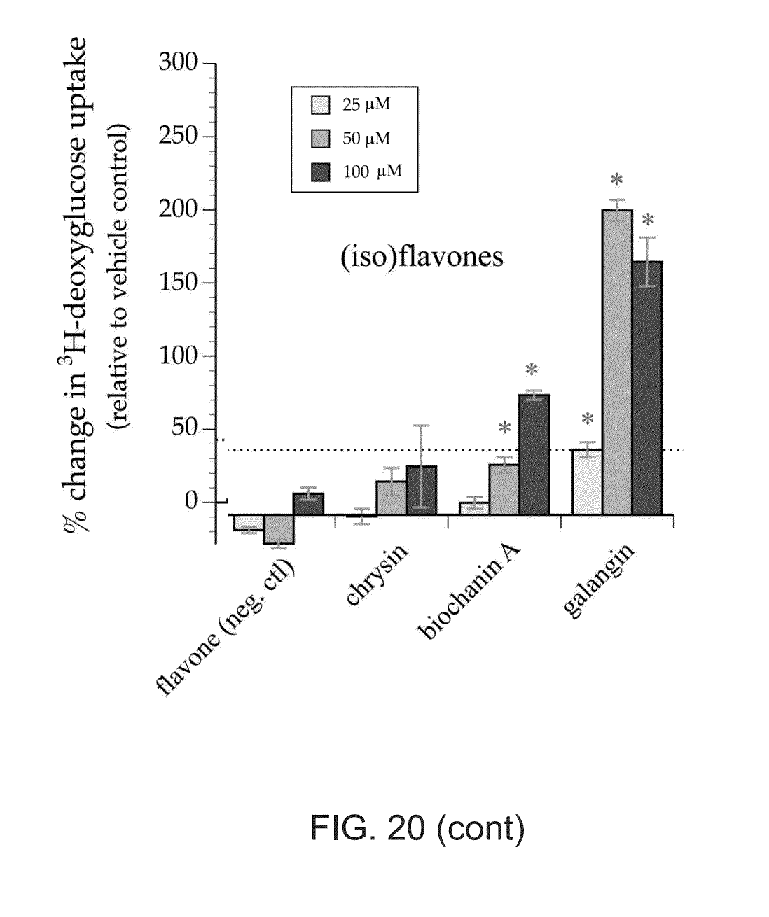

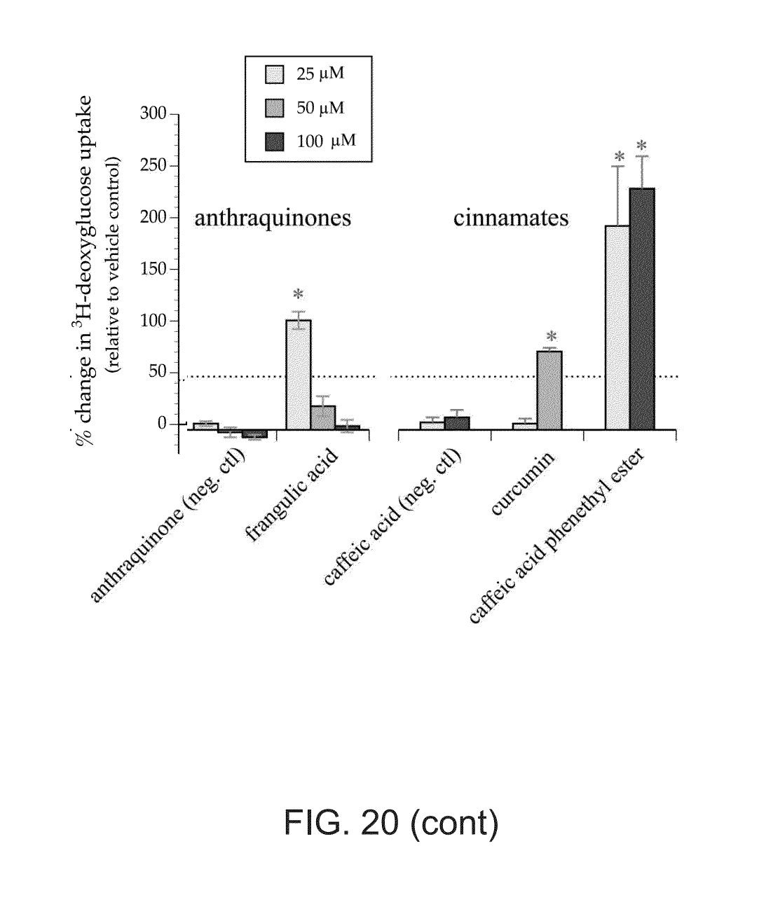

Nine of the compounds with significant uncoupling activity increased basal glucose uptake to levels greater than that achieved with metformin treatment at 400 μM (FIG. 20). Four of these compounds induced an effect of three- or more fold that of metformin. The most important effect, a 269% increase corresponding to over six times the effect of metformin at ⅛th of metformin's concentration, was induced by the chalconoid butein; this effect is believed to be the largest increase in glucose uptake reported to date in a muscle cell model. All nine actives were compounds that induced more than 50% uncoupling in isolated mitochondria at 100 μM, suggesting that a minimum threshold of uncoupling activity is required in order to promote significant upregulation of glucose uptake. Accordingly, and as expected, none of the 14 compounds with little or no uncoupling activity induced increases in glucose uptake superior to that of metformin. None of the treatment conditions caused morphological changes or other patent indications of cytotoxicity, save curcumin and kaempferol which respectively caused increased susceptibility to monolayer detachment and decreased uptake relative to vehicle-control levels at 100 μM (not shown).

Of the compounds with significant uncoupling activity, only caffeic acid phenethyl ester has previously been reported to increase muscle cell glucose uptake [85B]; this effect was subsequently found by our group to be related to uncoupling activity and the activation of AMPK signaling [37B]. Along this line, genistein, formononetin, and biochanin A have been found by others to stimulate mitochondrial biogenesis [86B], an effect concordant with AMPK activation. Also, derivatives of genistein have been shown to acutely induce very large increases in muscle cell glucose uptake that coincide with activation of AMPK and increases in mRNA content of GLUT1 and GLUT4 glucose transporters [87B].

It should be noted that three compounds of the present study, quercetin, resveratrol, and piceatannol, induced a modest enhancement of glucose uptake of 17 to 25% despite exhibiting little to no uncoupling activity. An increase in glucose uptake induced by resveratrol has previously been reported by others and found to be AMPK-dependent [19B], while our group has reported the effect of quercetin elsewhere and shown it to coincide with activation of AMPK signaling [20B]. Fittingly, quercetin, resveratrol, and piceatannol are all known inhibitors of ATP synthase [80B, 81B], and therefore, like compounds with uncoupling activity, their effects on glucose uptake are likely also the result of a perturbation of energy homeostasis. Along these lines, it is possible that the effect on glucose uptake of some uncouplers may be potentiated by concurrent inhibitory activity, as reported in Section 3.2.

While important enhancement of glucose uptake was clearly only induced by compounds exhibiting significant uncoupling activity, a strong correlation was not observed between glucose uptake and uncoupling. This should not be surprising given that uncoupling was assessed as an instantaneous effect in a reduced system (with the possibility of underestimation due to concurrent inhibitory activity, as considered in Section 3.2), whereas enhancement of glucose uptake represents the integration of a large number of sequential events over an 18 h period. Indeed, magnitude of enhancement of glucose uptake is likely related not only to magnitude of the perturbation of energy homeostasis, but also to onset time of the perturbation and, even more importantly, to its duration. Onset time of a given compound in cultured cells is likely determined by ease of transmembrane diffusion and aqueous diffusivity. Given the importance of ease of diffusion to magnitude of uncoupling activity, onset time is therefore likely related to magnitude of the perturbation of homeostasis, thereby introducing a non-linear component to the relationship between uncoupling activity and enhancement of glucose uptake. Duration of activity, on the other hand, may be a function of a compound's susceptibility to xenobiotic metabolism, including processes such as methylation, sulfanation, and glucuronidation that decrease lipophilicity or that target the hydroxyl groups critical to uncoupling activity, and that thereby abolish activity. Susceptibility to such transformations is likely determined by compound structure and factors such as molecular flexibility. As such, susceptibility can be expected to vary considerably across different chemical classes such as those represented here; on the basis of molecular flexibility alone, chalconoids and cinnamates may be expected to generally exhibit lesser susceptibility to metabolism than the more rigid flavonoids. If enhancement of glucose uptake is proposed to be related to the integral of perturbation of energy homeostasis over time, then duration of uncoupling activity can be considered to be as important a determinant of enhancement of glucose uptake as magnitude of uncoupling activity. By extension, if duration of activity is not taken into account either by direct measurement or by a structure-based prediction of susceptibility to metabolism, a strong correlation between uncoupling activity and enhancement of glucose uptake cannot be expected for a structurally-diverse set of compounds. Conversely, a good correlation is more likely to be observed for a highly homogeneous set of compounds, as was the case in our previous study of caffeic acid phenethyl ester and twenty related cinnamates which yielded a correlation of r²=0.80. [37B]. Finally, the threshold level of uncoupling activity for inducing significant enhancement of glucose uptake, estimated here to correspond to approximately 50% uncoupling in isolated mitochondria, is difficult to translate into % increase in oxygen consumption or % of oxidative capacity used in whole cells. However, this threshold is likely well below the level that can be expected to cause collapse of the mitochondrial membrane potential.

3.4 Suppression of glucose-6-phosphatase activity in H4IIE hepatocytes by identified uncouplers of the 4′-hydroxychalcone class:

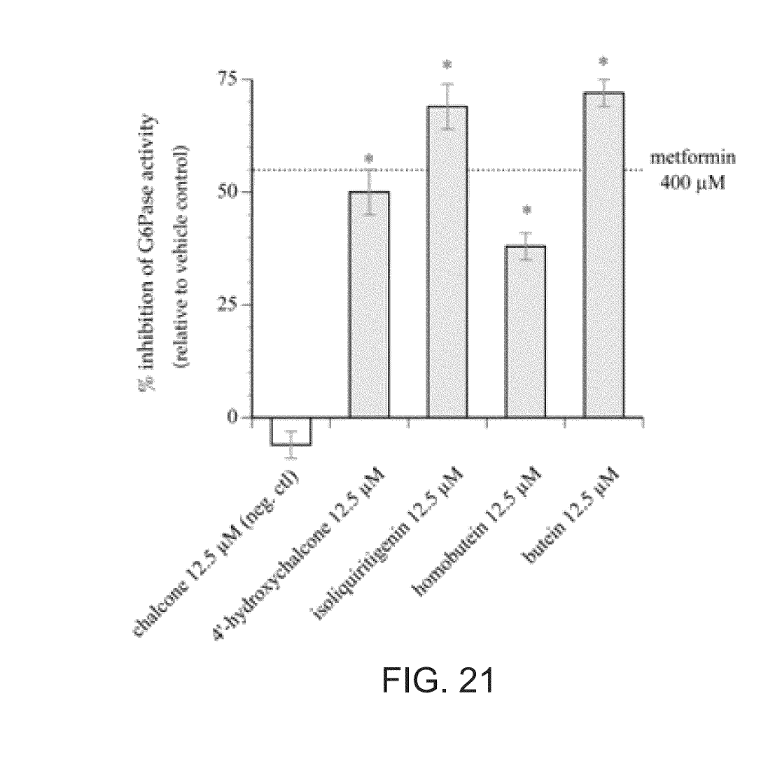

As a class, 4′-hydroxychalconoid uncouplers exhibited the most remarkable effects in muscle cells. These compounds and their unsubstituted parent, chalcone, were therefore selected for testing of suppression of G6Pase activity in H4IIE hepatocytes following a 16 h treatment, as induced by the reference uncoupler 2,4-dinitrophenol, above (Section 3.1.2). At 12.5 μM, all four compounds were more efficacious than insulin at 100 nM (FIG. 21). Butein and isoliquiritigenin were particularly active, both exceeding the effect of metformin at 400 μM. As expected, chalcone did not suppress G6Pase activity. None of the treatment conditions affected cellular viability, as assessed by rate of release of lactate dehydrogenase over 16 h. As before, G6Pase activity was normalized to each well's total protein content so as to account for any effects on rate of cellular proliferation. It should be noted that release of lactate dehydrogenase was increased by treatment with 4′-hydroxychalcone, isoliquiritigenin, and butein at 25 μM (not shown).

These findings and those of Section 3.1.2 represent the first report linking uncoupling activity to the therapeutically-relevant suppression of hepatic glucose output. Given that stimulation of glycogenolysis is an expected result of AMPK activation [94B], it is fitting that butein and isoliquiritigenin have been found by others to decrease liver glycogen content following 7-day administration in-vivo [95B]. Moreover, another 4′-hydroxychalconoid, 2′,4′-dihydroxy-4-methoxydihydrochalcone, has been found to induce an AMPK-dependent downregulation of phosphoenolpyruvate carboxykinase (PEPCK) and gluconeogenesis in liver cells [88B]. Finally, genistein, observed to exhibit significant uncoupling activity in Section 3.2, has been reported to decrease hyperglycemia through the suppression of G6Pase and PEPCK activities in vivo [89B]. It should be noted that several flavonoids with little to no uncoupling activity, as observed here, have nevertheless been reported by others to suppress G6Pase activity, glucose output, or gluconeogenesis. These include silibinin [90B], epigallocatechin gallate [91B], daidzein [89B], and naringenin [92B]. As most of these compounds were observed to induce important inhibition of oxidative phosphorylation in Section 3.2, their reported liver effects may therefore also be related to disruption of energy homeostasis.

3.5 Physicochemical Properties of Compounds Exhibiting Uncoupling Activity:

For a compound to possess Mitchellian uncoupling activity, it must exist in both neutral and ionized forms in both the mitochondrial intermembrane space (pH^{˜}7.4) and the mitochondrial matrix (pH^{˜}8.0) and be capable of diffusing across the mitochondrial inner membrane in either form. Because of differential speciation between the intermembrane space and the matrix, and, more importantly, because of the membrane potential across the mitochondrial inner membrane (^{˜}150 mV negative inside), the neutral species of such a compound is perpetually subjected to a concentration gradient into the matrix, while the less lipophilic negatively-charged species is perpetually subjected to an electrochemical gradient out of the matrix. (In the case of a basic compound, the neutral deprotonated species is subjected to a concentration gradient out of the matrix while the less lipophilic positively-charged species is subjected to an electrochemical gradient into the matrix). Consequently, under steady-state conditions, there is diffusion of protonated molecules into the matrix coupled to the diffusion of deprotonated molecules out of the matrix, the resulting cycle carrying a proton from the intermembrane space into the matrix down its electrochemical gradient with each iteration, and dissipating potential energy in the process. This mechanism accounts in whole for the activity of classical proton shuttle uncouplers and in part for that of protein-assisted uncouplers [30B-33B]. The efficiency with which a compound performs this cycle is clearly largely dependent on how easily the compound can diffuse through the mitochondrial inner membrane in neutral and in ionized form. This can be expected to be determined by compound lipophilicity [32B], such that diffusion efficiency is reduced by negative or positive deviations from an optimal degree of lipophilicity, and by the extent to which charge is delocalized, such that compounds capable of charge delocalization over a ring structure (e.g., phenolic compounds) exhibit a smaller reduction in lipophilicity upon ionization than compounds with more localized charge (e.g., carboxylic acids).

While a compound must clearly be ionizable and exhibit an acid-dissociation constant (pKa) within some 4 units above and below mitochondrial pH (i.e., pKa of approximately 4.0 to 11.4) in order to exist in neutral and ionized forms in both mitochondrial compartments (this range may be more constrained for compounds with two or more groups ionizable at physiological pH), it is unclear whether pKa is otherwise related to activity. Similarly, while the importance of lipophilicity is clear, the optimum degree of lipophilicity or even the range of lipophilicity compatible with Mitchellian uncoupling are not well-defined. Efforts to elucidate the relationship between physicochemical properties and activity may be confounded by chemical-class-dependent differences. Alternatively, they may be confounded by a failure to distinguish classical proton shuttle uncouplers from protein-assisted uncouplers. Indeed, although all uncouplers are subject to the same fundamental constraints of pKa and lipophilicity, it is likely that interaction of an uncoupler with a protein target imposes additional constraints, structural and physicochemical; by extension, a compound well-suited for interaction with its target protein may exhibit physicochemical properties that are sub-optimal for Mitchellian uncoupling. Given the homogeneity of the present study's testset and the likelihood that compounds of interest are classical proton shuttle uncouplers, examination of the testset as a whole may provide new insight into physicochemical properties most conducive to Mitchellian uncoupling.

Calculated values of pKa₍₁₎ (the acid-dissociation constant of the most readily ionizable site) and of the octanol-water partition coefficient (P_{octanol-water}; a well-established measure of lipophilicity) are listed in Table 2 for all compounds. From these, it can be appreciated that activity is indeed compatible with an extensive range of both pKa₍₁₎ and log P_{octanol-water} values; the fourteen most active compounds were characterized by a pKa₍₁₎ ranging from 4.5 to 9.2 and log P_{octanol-water} ranging from 2.5 to 4.1. As expected, all compounds exhibiting uncoupling activity were characterized by one or more ionizable hydroxyl groups, and unsubstituted class parent compounds (flavone, chalcone, anthraquinone, and stilbene) were accordingly devoid of activity. Although only a small number of compounds of the testset fell outside the pKa₍₁₎ range of 4.0 to 11.4 considered compatible with activity, all of these, save one, were also devoid of activity. The exception, salicylic acid, like many carboxylic acids, is known to dimerize through hydrogen bonding between carboxyl groups; dimerization effectively renders the carboxyl group nonionizable and the apparent pKa₍₁₎ of the dimer (estimated at 5.5) can therefore be expected to be higher than that of the monomer (calculated at 2.8).

Hard cut-off values for log P_{octanol-water} are not appropriate since any compound exhibiting the necessary acid-dissociation behavior and of the mass range of the compounds considered here can be expected to exhibit some degree of membrane permeability and therefore some degree of Mitchellian uncoupling activity. Therefore, any proposed cut-off values must be specific to a reference test concentration. Based on the large number of low to moderate lipophilicity compounds considered, a log P_{octanol-water} value of approximately 1.8 can be proposed as a lower cut-off for measurable activity at 100 μM. An upper cut-off value, however, cannot be identified from the present testset due to an insufficient number of highly lipophilic test compounds. It should be noted that the reference uncoupler 2,4-dinitrophenol falls below the proposed lower limit. However, this compound is known to dimerize through hydrogen bonding between a nitro and a hydroxyl substituent, resulting in an approximate doubling of the effective log P_{octanol-value} of the dimer relative to the monomer.

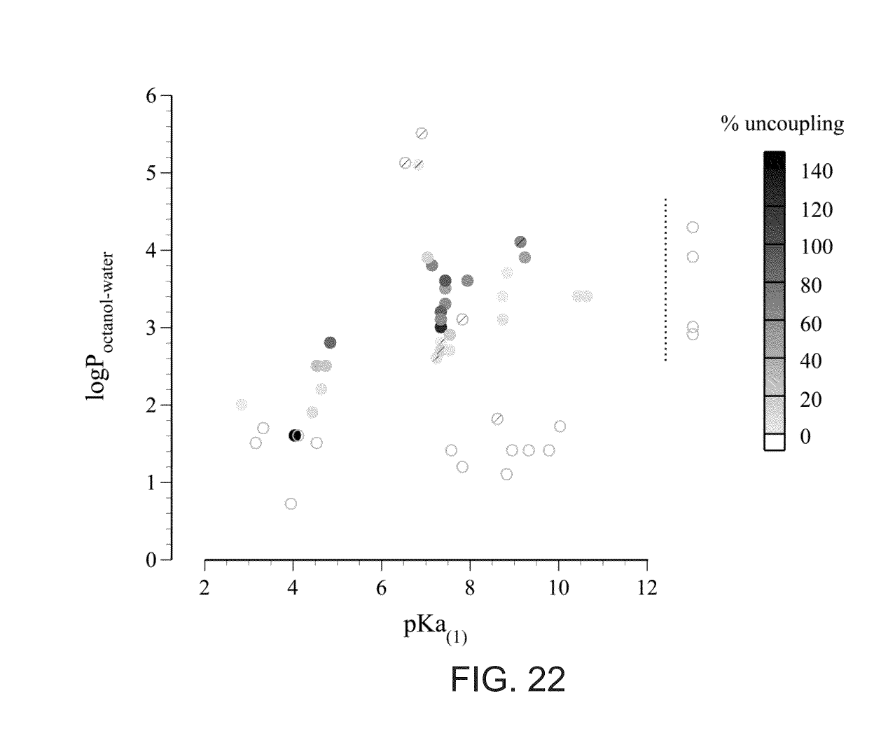

It is difficult to identify an optimal value of log P_{octanol-water} given that activity appears unrelated to lipophilicity among the 14 most active compounds of the testset; while activity is indeed well-correlated to lipophilicity at lower values of lipophilicity, as best appreciated from the six members of the flavonol subclass spanning a range of log P_{octanol-water} from 1.5 to 2.8 and a range of uncoupling activity from 1% to complete uncoupling, the five compounds that induce complete uncoupling span the considerable range of log P_{octanol-water} of 2.8 to 3.8. This may be reconciled if the proposed bell-shaped relationship between lipophilicity and activity is characterized by a plateau rather than a peak optimum. Alternatively, identification of an optimal value of log P_{octanol-water} may require that pKa be taken into account. Indeed, if acid-dissociation behavior is considered alongside lipophilicity, as in the three-dimensional plot of FIG. 22, then a relationship may be proposed such that compounds with suboptimal lipophilicity can nevertheless exhibit significant uncoupling activity if their pKa tends towards the lower limit of compatibility. This notion is best appreciated by comparing the flavones and isoflavones of the testset, in all of which the position 7 hydroxyl substituent is the most readily ionizable and imparts a pKa₍₁₎ ranging from 7.3-7.5, to flavonols, characterized by a position 3 hydroxyl substituent that confers a pKa₍₁₎ on the order of 5.0: significant uncoupling activity is observed in flavones and isoflavones that exhibit values of log P_{octanol-water} between 2.9 to 3.2 and activity falls off abruptly in compounds with only slightly lower lipophilicity; in contrast, flavonols with log P_{octanol-water} of 2.5 to 2.8 exhibit activity comparable to (iso)flavones with log P_{octanol-water} of 2.9 to 3.2. The difference is underscored by the fact that flavonols exhibit a decrease in log P_{octanol-water} of ^{˜}3.5 units upon ionization due to poor charge delocalization over the flavonoid chroman ring, rather than the ^{˜}2 unit decrease characteristic of delocalization over a benzene ring; in sharp contrast to log P_{octanol-water} values on the order of 1.0 for the ionized species of the active flavones and isoflavones, the ionized species of the active flavonols exhibit values of on the order of −1.0. Another example comes from the comparison of the 4-hydroxychalconoids isoliquiritigenin (pKa₍₁₎ value of 7.4) and 4′-hydroxychalcone (pKa value of 7.9); in spite of an identical log P_{octanol-water} value of 3.6, isoliquiritigenin exhibits greater uncoupling activity than its counterpart. This would suggest not only that pKa is indeed a determinant of uncoupling activity, with values below mitochondrial pH most conducive to activity, but also that pKa and lipophilicity are non-independent predictors of activity. By extension, a pKa above mitochondrial pH may hinder activity. Indeed, among the fourteen compounds with significant uncoupling activity none are characterized by a pKa₍₁₎ value greater than 8.0, save caffeic acid phenethyl ester, shown elsewhere to represent a family of uncouplers with atypical structural constraints [37B], and the related compound curcumin, shown by others to be an inducer of mitochondrial permeability transition rather than an uncoupler [77B, 93B]. Moreover, a number of compounds of the testset characterized by appropriate lipophilicity but a pKa₍₁₎ value on the order of 9.0, namely compounds of the stilbenoid class, exhibited negligible uncoupling activity. It is unclear how a mitochondrial distribution of uncoupler molecules skewed in favor of the ionized species would be more conducive to activity than a more balanced distribution expected from a pKa₍₁₎ value near mitochondrial pH. Nevertheless, that lower pKa values are more conducive to activity is in accord with long-standing empirical observations that reference uncouplers such as 2,4-dinitrophenol (pKa^{˜}4.0), carbonyl cyanide 3-chlorophenylhydrazone (CCCP; pKa^{˜}6.0) and carbonyl cyanide 4-(trifluoromethoxy) phenylhydrazone (FCCP; pKa^{˜}6.2) tend to be characterized by pKa below mitochondrial pH, although this must be tempered by the fact that most reference uncouplers are protein-assisted uncouplers. As proposed, a contribution of pKa to activity would narrow the optimal value of log P_{octanol-water} to within a range of 3.0 to 3.6.

In addition to lipophilicity, the ease with which a compound diffuses across the mitochondrial inner membrane, and hence its uncoupling activity, can be expected to be determined by geometric considerations. Indeed, other properties being equal, a small compound will diffuse more readily than a larger compound. Large size may therefore account for the low activity of some compounds considered here, such as the flavonoid C—C dimers (i.e., bisflavonoids) and other complex flavonoids such as silibinin, as none of the most active compounds save curcumin have a molecular mass of more than 300 Da. It should, however, be noted that compound size and lipophilicity co-vary and become increasingly related at higher size/lipophilicity, making it difficult to estimate the contribution of excessive size to low uncoupling activity; indeed, excessive lipophilicity may have contributed as much as geometric considerations to the lack of the activity of the bisflavonoids. Perhaps more important than compound size is shape, an independent predictor of activity such that planar and linear compounds intercalate membrane phospholipids more easily than non-planar or globular compounds of similar mass and other properties. In support of this, it is observed that all 14 compounds with significant activity exhibit a high degree of, or complete planarity. Moreover, deviation from planarity resulting from dehydrogenation of either the flavonoid chroman ring, such as in flavanones and flavanols, or of the chalconoid backbone, as in the dihydrochalconoid phloretin, appears to significantly impact uncoupling activity. The effect of deviation from planarity is perhaps most evident in epigallocatechin gallate, a distinctly non-planar compound devoid of activity in spite of compatible acid-dissociation behavior (pKa₍₁₎ value of 7.8) and near optimal lipophilicity (log P_{octanol-water} value of 3.1). Predicted deviations of compound shape from planarity are reported in Table 2 and are indicated in FIG. 22. The notion that planarity is an important attribute for uncoupling activity has been suggested by others also focusing on the uncoupling activity of naturally-occurring compounds [71B]; specifically, it was suggested that imperfect planarity was a contributor to the diminished activity of flavanones relative to flavones, as also observed here. Interestingly, it has been proposed that bulky substituents may be favorable to uncoupling activity if these are positioned in such as way as to “shield” the ionizable group [32B], presumably reducing solvent accessibility to that part of the molecule. However, in the presence of extensive charge delocalization, it is questionable whether bulky substituents can further minimize the decrease in lipophilicity incurred upon ionization.

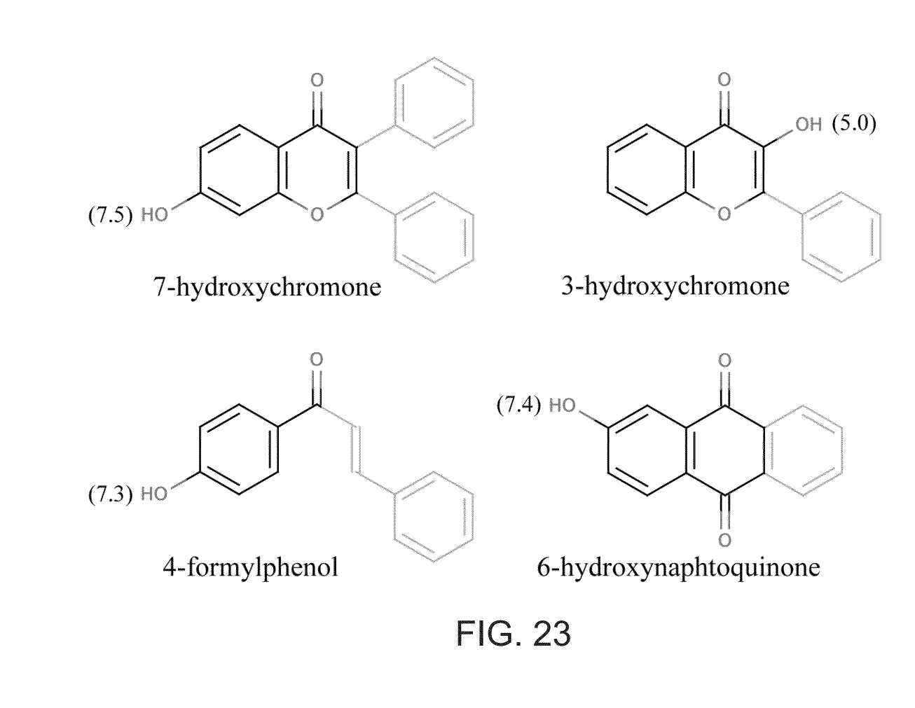

Regardless of the exact nature of the relationship between pKa and uncoupling activity, four parent structural templates (FIG. 23) emerge from the screening study as particularly conducive to uncoupling activity on the basis of their intrinsic pKa and planarity: the 3-hydroxyflavone structure (i.e., flavonol), or its simpler 3-hydroxychromone root (pKa^{˜}5.0); the 7-hydroxy(iso)flavone structure, or its simpler 7-hydroxychromone root (pKa^{˜}7.5); the 4′-hydroxychalconoid structure, or its simpler 4-formylphenol root (pKa^{˜}7.3); and the 2-hydroxyanthraquinone structure, or its simpler 6-hydroxynaphtoquinone root (pKa^{˜}7.4). For the most part, these templates are insufficiently lipophilic to exhibit significant uncoupling activity on their own. This is supported by the lack of activity exhibited by 4-acetylphenol and 7-hydroxychromone in Section 3.2. However, compounds derived from these templates have a high probability of being active if they are substituted so as to exhibit a log P_{octanol-water} value between 3.0 and 3.6 and so as to conserve a planar structure. These templates are common in plant metabolites and it is therefore to be expected from this that proton shuttle uncouplers are highly prevalent in nature. It should be noted that compounds of the types screened in this study occur more frequently in nature as glycosides than aglycones. However, because the sugar moiety of glycosides typically reduces lipophilicity by some three units of log P_{octanol-water} while also adding considerable molecular bulk, glycosides can generally be expected to be poor uncouplers.

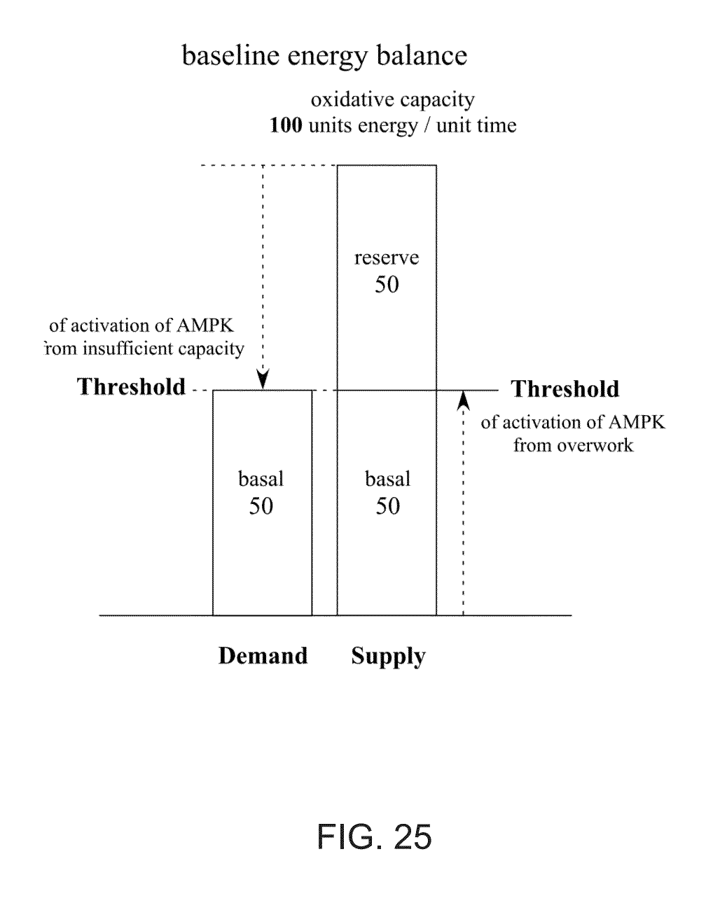

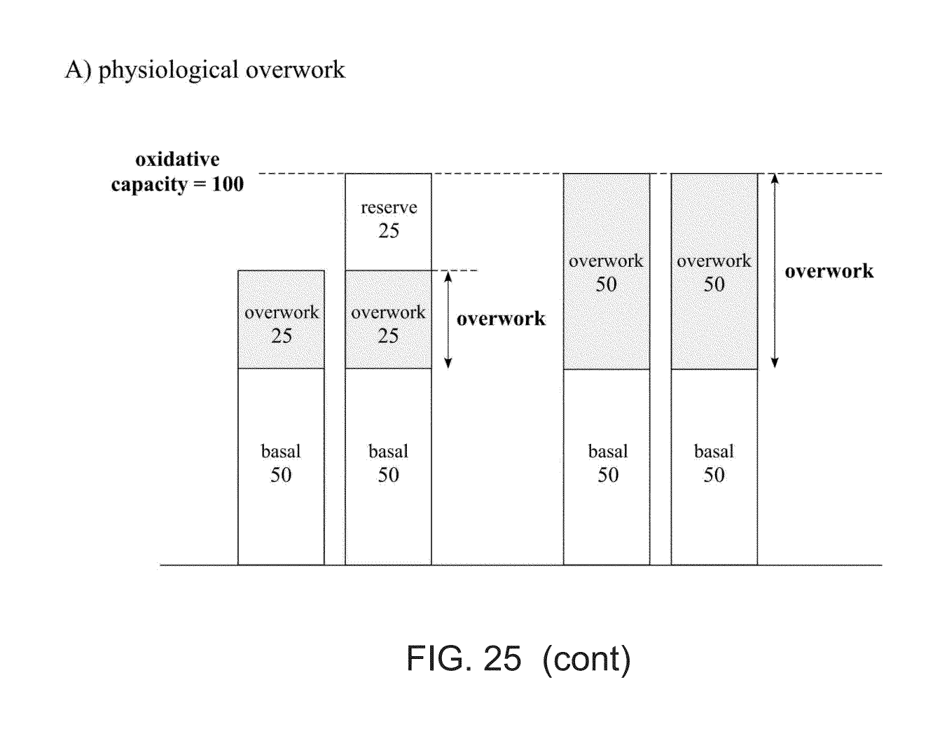

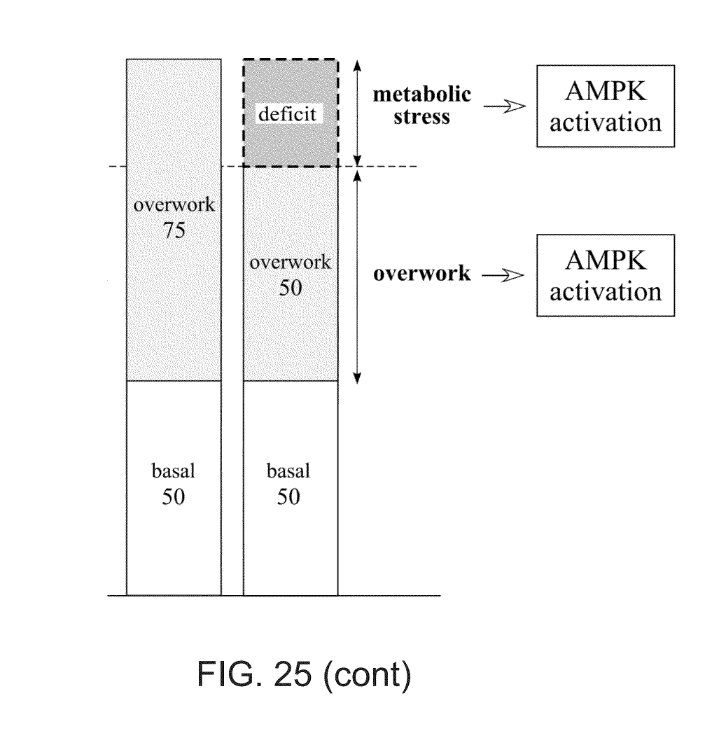

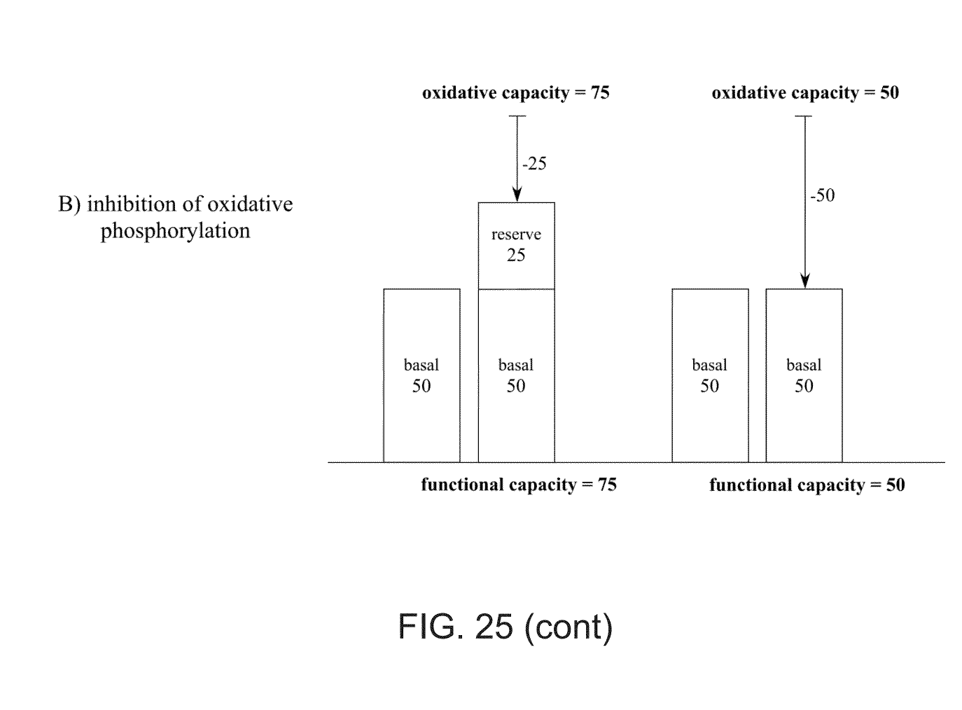

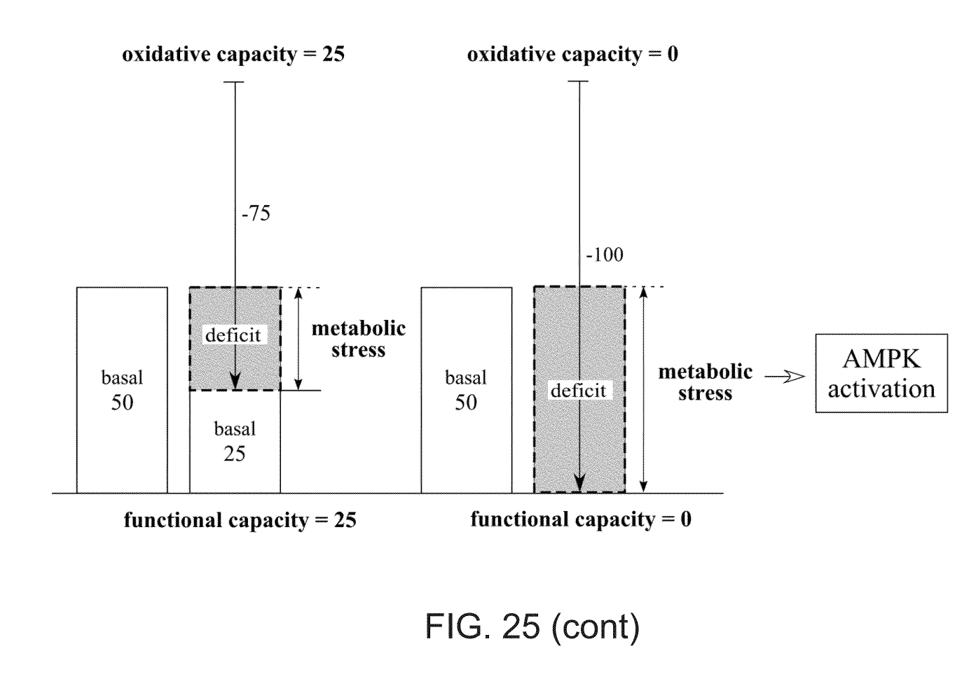

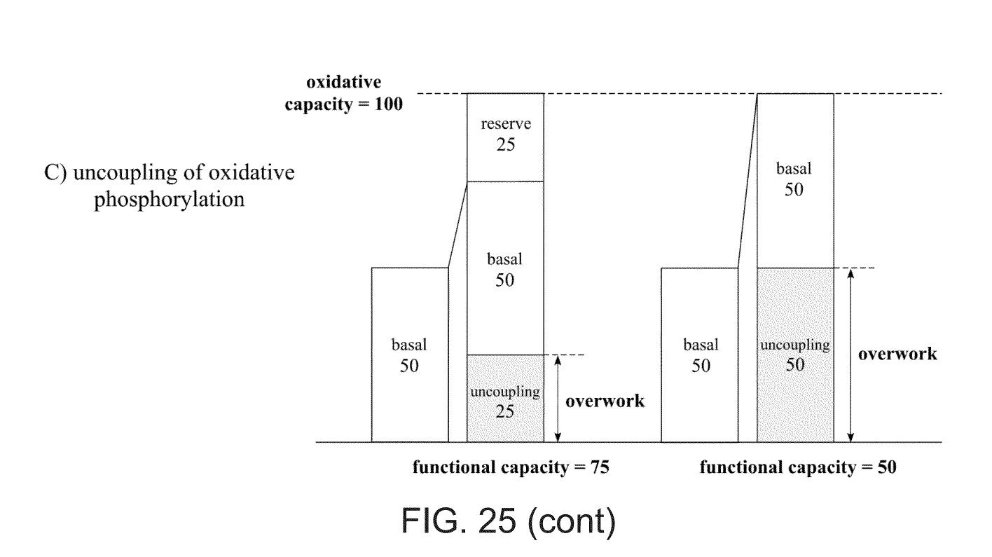

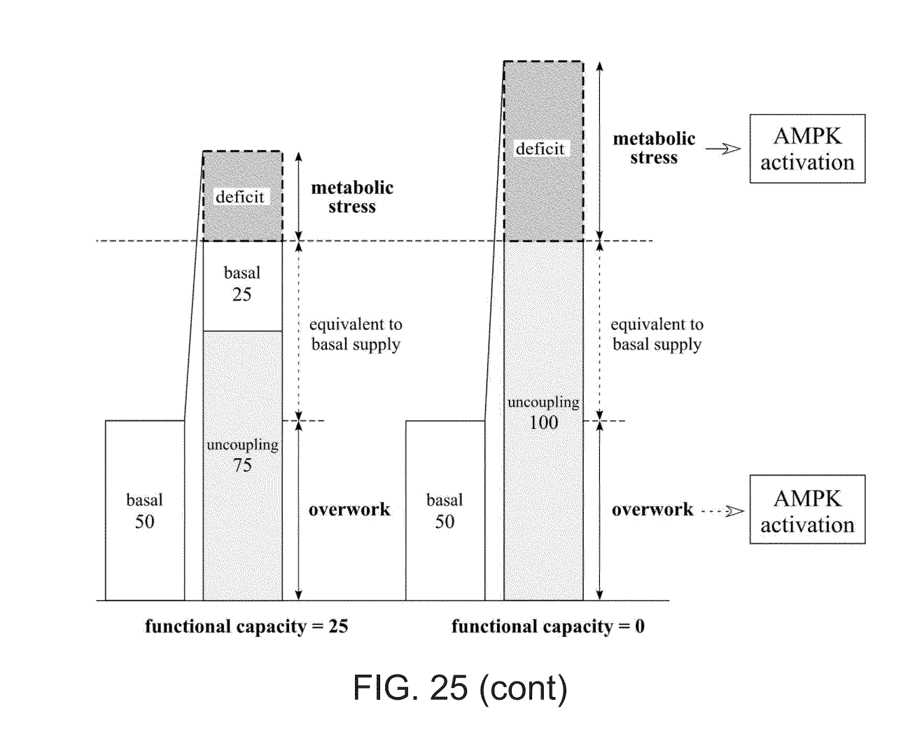

4. Summary: This study demonstrates that compounds exhibiting uncoupling activity can induce an important enhancement of glucose uptake capacity in skeletal muscle cells, greatly surpassing that induced by metformin, and an important insulin-like suppression of glucose output in hepatocytes, at least comparable to that induced by metformin; these activities are of clear importance to the control of hyperglycemia. These findings are consistent with a mechanism of action whereby the reduced metabolic efficiency induced by a transient uncoupler-mediated proton leak across the mitochondrial inner membrane of insufficient magnitude to collapse the membrane potential causes a perturbation of energy homeostasis that triggers AMPK signaling and leads to, among other beneficial AMPK-mediated effects, the upregulation of GLUT1 and GLUT4 transporters in muscle cells and the downregulation of key enzymes of gluconeogenesis and glucose output in hepatocytes. A wide variety of compounds have been used for this demonstration, including the reference weak uncoupler 2,4-dinitrophenol and numerous phenolic compounds of plant origin observed to exhibit uncoupling activity in isolated mitochondria, in many cases comparable to that of 2,4-dinitrophenol. To reinforce the link between uncoupling and these activities of therapeutic interest, a large number of related compounds exhibiting little to no uncoupling activity were shown to have negligible effect on upregulation of glucose uptake. Structure-activity analysis of screened compounds has provided insight into optima and limits of physicochemical properties compatible with uncoupling activity. Moreover, actives have been reduced to a handful of structural templates common in plant metabolites and here proposed to be particularly conducive to uncoupling activity. An imperfect relationship between magnitude of uncoupling activity and enhancement of glucose uptake suggests that other factors such as time to onset and duration of activity also influence the endpoint. A minimum threshold level of activity (or of the integral of activity over time) may also be required. Finally, although uncouplers were only compared to metformin in the present study, results nevertheless lend support to the hypothesis that uncoupling may be a more effective mechanism than inhibition of oxidative phosphorylation for inducing effects of relevance to insulin resistance in tissues with high oxidative capacity such as skeletal muscle; a physiological explanation for such an advantage of uncouplers over inhibitors like metformin is proposed in FIG. 25. The findings of this study have applications to the identification or design of safe and novel therapies against insulin resistance and related metabolic disorders based on short-lived compounds with weak uncoupling activity.

Referring to FIG. 17: Enhancement of Skeletal Muscle Cell Basal Glucose Uptake and Suppression of Hepatocyte Glucose-6-Phosphatase (G6Pase) Activity by the Reference Weak Uncoupler 2,4-Dinitrophenol. A) Treatment of differentiated C2C12 muscle cells for 18 h with 2,4-dinitrophenol

(structure inset) resulted in an important and dose-dependent increase in the rate of constitutive, non-insulin-stimulated glucose uptake, as assessed by incorporation of radiolabeled deoxyglucose over 10 min. At 50 to 100 μM, 2,4-dinitrophenol more than doubled the absolute rate of basal glucose uptake. This effect was nearly four-fold greater than could be achieved with the anti-diabetic drug metformin at 400 μM or by its more potent derivative phenformin at 100 μM, and more than six-fold greater than could be induced by a 30 min treatment with a supra-physiological concentration of insulin. Sustained increases in rate of basal glucose uptake above that of insulin-stimulated uptake, as induced here by treatment with 2,4-dinitrophenol or biguanides, are indicative of an increase in the cellular capacity for uptake. Whereas acute insulin-like effects of 2,4-dinitrophenol on glucose uptake have been reported by others, such longer-term and quantitatively more important effects have not been attributed to 2,4-dinitrophenol or other uncouplers of oxidative phosphorylation. Results are expressed as mean±SEM increase in deoxyglucose uptake relative to a vehicle-treated control group for three or more independent experiments of three to four replicates per condition per experiment. * denotes a significant difference (p≦0.05) from the respective vehicle control group. Cellular morphology was unaffected by any of the treatment conditions. B) The increase in rate of basal glucose uptake induced by an 18 h treatment with 2,4-dinitrophenol or with metformin was greater than that induced by a 3 h treatment in parallel cells pre-treated for 15 h with vehicle. Additionally, repeat treatment 3 h before the end of the 18 h treatment did not further increase the rate of basal glucose uptake relative to that resulting from the simple 18 h treatment. That several hours are needed for the full expression of the effect of 2,4-dinitrophenol or of metformin is again consistent with an enhancement of glucose uptake capacity through increased expression of effector proteins. On the other hand, the increases observed to result from the 3 h treatment, on the order of that which can be induced by insulin stimulation (A), may be explained by transient post-translational effects (i.e., translocation and/or activation of glucose transporters). Results shown were generated from a single experiment in which all three treatment paradigms were performed in parallel on cells of the same passage, with all conditions were included on each plate. Results are expressed as mean+SEM counts per minute of incorporated label for four replicates per condition. * denotes a significant difference (p≦0.05) from the respective vehicle-treated control group for each paradigm. C) Treatment of H4IIE hepatocytes with 2,4-dinitrophenol induces insulin-like and metformin-like suppression of G6Pase activity. G6Pase is rate-limiting to the release of glucose by hepatocytes and its expression is tightly regulated by insulin. All treatments were for 16 h. This insulin-like effect of metformin accounts for much of the drug's anti-hyperglycemic activity. However, this insulin-like activity has not previously been attributed to 2,4-dinitrophenol or other uncouplers of oxidative phosphorylation. Results are expressed as mean % change+SEM, relative to a vehicle-treated control group for two independent experiments of four to six replicates per condition. Viability was unaffected by any of the treatment conditions, as assessed by release of lactate dehydrogenase (not shown). To further control against potentially confounding effects of cytotoxicity or of altered rate of proliferation, G6Pase activity is normalized by total protein content on a well by well basis. * denotes a significant difference (p≦0.05) from the vehicle control group. Dexamethasone (1 μM), used as a negative control, increased G6Pase activity by 56±10% (not shown).

Referring to FIG. 18: Chemical Structure of 50 Naturally-Occurring Phenolic Compounds Screened for Uncoupling Activity. Compounds were selected so as to represent several phytochemical classes, including flavonoids (6 subclasses), chalconoids, stilbenoids, anthraquinones, and simple phenolics, spanning a wide range of structure and physicochemical properties. All compounds considered are composed exclusively of C, H, and O. The unsubstituted parent compound for each of the polycyclic classes, nonionizable at physiological pH and therefore devoid of uncoupling activity, are included as negative controls throughout the present work. Compounds are identified by traditional phytochemical nomenclature. CAS registry numbers are reported in Table 2. The substituent numbering convention is indicated for the prototypical compound of each class. Calculated acid-dissociation constants are indicated beside each ionizable group. Compounds with bolded names are most active uncouplers, inducing more than 20% uncoupling at 100 μM in isolated rat liver mitochondria, as reported in Table 2.

Referring to FIG. 19: Representative Oxygen Consumption Tracings from Isolated Mitochondria Illustrating the Instantaneous Increase in the Rate of Basal Oxygen Consumption (state 4 respiration; non-ADP-stimulated) Characteristic of Uncoupling Activity. Action of the reference uncoupler 2,4-dinitrophenol and of two 4′-hydroxychalconoids of the screening testset, butein and homobutein, are illustrated. Compounds were applied at 50 μM in 0.1% DMSO. Respiration was supported by the complex II substrate succinate. Basal oxygen consumption was monitored for 2 min prior to injection of test compound or vehicle (indicated by black arrow) and then for at least 1 min thereafter. State 3 respiration was then initiated by the injection of 200 μM ADP (final concentration; indicated by white arrow). Rates of oxygen consumption in nmoles/min/mg mitochondrial protein are indicated under each segment of the tracings. Butein and homobutein induced partial uncoupling at this concentration (doubling the rate of basal oxygen consumption), whereas 2,4-dinitrophenol increased basal oxygen consumption above the level of ADP-stimulated consumption of vehicle-treated mitochondria of the same preparation (defined as 100% uncoupling). Chalcone, the parent compound of the two 4′-hydroxychalconoid uncouplers, was devoid of uncoupling activity in accordance with its lack of ionizable site. However, this compound exerted a small inhibitory effect on oxidative phosphorylation, observable as reductions in the rates of both basal and ADP-stimulated oxygen consumption. Inhibition of ATP synthase or other enzyme complexes of oxidative phosphorylation are common activities of naturally-occurring phenolic compounds. In the present case, inhibition in the absence of ADP and the use of succinate as substrate point to inhibition between complex II and IV of the electron transport chain or inhibition of substrate transport. Butein and homobutein also exhibited inhibition of ADP-stimulated consumption and it is likely that inhibition of basal oxygen consumption was also present but masked by the uncoupling activity, thereby resulting in a minor underestimation of uncoupling activity. It should be noted that the term uncoupling normally applies specifically to protonophoric activity, but that an increase in the rate basal oxygen consumption as measured here can be the result of broader cationic activity. Moreover, the methodology used does not distinguish between classical proton shuttle uncoupling (i.e., unassisted Mitchellian uncoupling) and uncoupling facilitated by interaction with protein constituents of the mitochondrial inner membrane. However, on the basis of structure and physicochemical characteristics, it can be expected that the majority of compounds considered in the present study exhibit classical proton shuttle uncoupling activity.

Referring to FIG. 20: (A) Relationship Between Uncoupling in Isolated Mitochondria and Upregulation of Glucose Uptake in Skeletal Muscle Cells. In addition to the reference uncoupler 2,4-dinitrophenol, fourteen naturally-occurring compounds found to induce more than 20% uncoupling at 100 μM in isolated rat liver mitochondria, and fourteen related compounds with little to no uncoupling activity under the same conditions were tested in C2C12 muscle cells for upregulation of glucose uptake activity following an 18 h treatment at 25 to 100 μM. Uncoupling activity is presented on the left-side x-axis and glucose uptake upregulation activity is presented on the right. Compounds are listed in decreasing order of uncoupling activity measured at 100 μM. For reference, the magnitude of increase in glucose uptake induced by 30 min of insulin stimulation (100 nM) and by an 18 h treatment with 400 metformin, as reported in FIG. 1, are indicated by dashed lines. Uncoupling is reported as increase in rate of basal oxygen consumption (state 4 respiration), expressed as % of the average rate of ADP-stimulated consumption (state 3 respiration) measured in vehicle (0.1% DMSO)-treated mitochondria of the same preparation. Each compound was tested in 2-3 independent mitochondrial preparations. Results are expressed as mean±SEM. Glucose uptake results are expressed as mean+SEM % increase in deoxyglucose uptake relative to vehicle-treated controls for three or more independent experiments of three to four replicates per condition per experiment. * denotes a significant difference (p 0.05) from vehicle (0.1% DMSO)-treated controls (SEM=2). All conditions were confirmed not to cause morphological changes or other patent indications of cytotoxicity, with the exception of curcumin, which increased susceptibility to monolayer detachment at 100 μM (not shown), and of kaempferol, which decreased uptake relative to vehicle-control levels at 100 μM (not shown). CAPE=caffeic acid phenethyl ester. EGCG=epigallocatechin gallate. (B) Complete glucose uptake dataset for compounds of interest and their respective nonionizable class parent compound devoid of uncoupling activity, sorted by class. Results are expressed as above. Cellular morphology was unaffected by any of the treatment conditions.

Referring to FIG. 21: Powerful Insulin-Like and Metformin-Like Suppression of G6Pase Activity in H4IIE Hepatocytes Induced by Uncouplers of the 4′-Hydroxychalconoid Family. The class parent compound chalcone, devoid of uncoupling activity, is included as a negative control. All uncouplers were tested at 12.5 μM. Treatment duration was 16 h. The dashed line indicates the level of suppression achieved by 400 μM metformin over the same treatment duration, as reported in FIG. 1C. Results are expressed as mean % change±SEM, relative to a vehicle-treated control group for two independent experiments of four to six replicates per condition. Activity is normalized by each well's total protein content. * denotes a significant difference (p 0.05) from the vehicle control group. Release of lactate dehydrogenase over 16 h was not increased by any of the treatments.

Referring to FIG. 22: Uncoupling Activity of 2,4-Dinitrophenol and 50 Screened Compounds

Plotted Against Compound Acid-Dissociation Behavior (pKa₍₁₎) and Lipophilicity (log P_{octanol-water} value of the protonated species), Two Main Determinants of Uncoupling Activity. Values of pKa₍₁₎ and log P_{octanol-water}, calculated using commercial chemoinformatics software, are reported in Table 2. Uncoupling is reported as mean increase in rate of basal oxygen consumption (state 4 respiration), expressed as % of the average rate of ADP-stimulated consumption (state 3 respiration) measured in vehicle (0.1% DMSO)-treated mitochondria of the same preparation. Compounds predicted to exhibit a three dimensional shape that markedly deviates from planarity are identified by a diagonal line through their symbol; deviations from planarity are reported in Table 2.

Referring to FIG. 23: Core Structures Conferring Activity to Identified Uncouplers. The fourteen compounds found to exhibit significant uncoupling activity can be reduced to four hydroxylated core structures characterized by appropriate acid-dissociation properties. Calculated pKa is indicated beside each hydroxyl group. It is proposed that all monoprotic compounds and many multiprotic compounds containing one of these structures will also exhibit significant uncoupling activity if the substituents to the core structure confer an appropriate degree of lipophilicity (i.e., log P_{octanol-water} on the order of 2.8 to 3.6) and if compound three-dimensional structure is planar or nearly-planar; nitro, cyano, or other groups typically associated with uncouplers are not required for activity. Given that these core structures are common in plant metabolites, the prevalence of naturally-occurring uncouplers may be higher than previously appreciated. These core structures may also serve as templates for the design of synthetic uncouplers for the indirect activation of AMPK; by restricting design to derivatives composed exclusively of C, H, and O, and by not exceeding the optimal degree of lipophilicity for activity, safety of such synthetic compounds can be maximized.

Referring to FIG. 24: Concentration-Activity Relationship of 2,4-Dinitrophenol and of Fourteen Compounds of the Screening Testset Exhibiting Greatest Uncoupling Activity at 100 μM. Results of a representative dose-escalation experiment in isolated rat liver mitochondria for each of the compounds are plotted on logarithmic scales. Uncoupling is reported as increase in rate of basal oxygen consumption (state 4 respiration), expressed as % of the average rate of ADP-stimulated consumption (state 3 respiration) measured in vehicle (0.1% DMSO)-treated mitochondria of the same preparation. DMSO was confirmed to have no effect on basal oxygen consumption at concentrations of up to 2% under the dose-escalation paradigm. Concentration at which 50% uncoupling is induced (U₅₀) and maximal inducible uncoupling are reported in Table 2. Slope of the linear portion of the relationship are indicated on each plot.

Referring to FIG. 25: Proposed Distinction Between Inhibition and Uncoupling of Oxidative Phosphorylation as Mechanisms for Perturbing Energy Homeostasis for the Indirect Activation of AMPK. It can be expected that an inhibitor of the electron transport chain or of ATP synthase does not induce activation of AMPK unless the reduction in oxidative capacity that it causes is of sufficient magnitude that cellular energy needs cannot be met by the residual capacity; only beyond this threshold is the cellular ATP concentration compromised and energy homeostasis therefore perturbed. Note that this threshold for metabolic stress is determined by the ratio of basal energy demand to oxidative capacity (or, alternatively, the magnitude of reserve capacity), which varies greatly between cell types. Activation of AMPK signaling can be expected to be directly related to the magnitude of mismatch between supply and demand, up to the point of zero residual capacity. Note that a deficit need not result in depletion of cellular ATP if it is compensated by anaerobic glycolysis, upregulated allosterically by the decreased concentration of ATP and the increased concentrations of ADP and AMP. Moreover, the activation of AMPK decreases basal energy demand through inhibition of non-essential energy-consuming cellular functions, thereby reducing the deficit. Similarly to an inhibitor, an uncoupler of oxidative phosphorylation induces metabolic stress when the reduction in functional capacity that it causes is of sufficiently great magnitude that the effective maximal rate of ATP resynthesis is lower than the rate of ATP consumption. However, even if the reduction in functional capacity is insufficient to cause such deficit, uncoupling may still be considered to perturb energy homeostasis. Indeed, the decrease in energy transduction efficiency resulting from uncoupling, whereby less ATP is resynthesized at a given rate of mitochondrial respiration, translates into an increased cost to basal activity. Alternatively, the increase in oxidation needed at a given rate of ATP resynthesis must be supported by energy-consuming processes, such as substrate import into the mitochondrion, amounting to an indirect increase in ATP demand. It is therefore proposed that uncoupling induces AMPK activation through overwork, regardless of whether a deficit between supply and demand exists, and that this activation is additive to that induced by deficit. In skeletal muscle where oxidative capacity is very high and resting energy demand is met by a small fraction of the capacity required for contractile activity (i.e., large reserve capacity), uncoupling may be a more effective mechanism for activating AMPK than inhibition. Moreover, activating AMPK without inducing metabolic stress may greatly minimize the risk of complications associated with indirect activators of AMPK. These notions are presented graphically in terms of energy demand and supply for a hypothetical system characterized by an arbitrary oxidative capacity of 100 energy units per unit time and a resting energy demand of 50 energy units per unit time. For simplicity, the contribution of anaerobic glycolysis is ignored and basal demand is considered to be a constant. Perturbation of energy homeostasis, and hence activation of AMPK, can be considered to occur if capacity fails to meet demand (i.e., deficit; metabolic stress) or if demand increases above the level of basal demand (i.e., overwork). This is most intuitive in a situation of physiological overwork (e.g., contractile activity in muscle cells), as depicted in A, where demand is increased by 25 (left), 50 (center), or 75 units (right). In B, oxidative capacity of the system is decreased through inhibition of oxidative phosphorylation; in this situation, AMPK is activated only when capacity falls below 50 units. In C, functional capacity of the system, rather than its actual capacity, is decreased by uncoupling. This is depicted as a greater proportion of supply attributed to meet the basal demand. AMPK is proposed to be activated by this overwork-like effect of uncoupling, in addition to the deficit depicted in the rightmost two panels. Note that if oxidative capacity is not overwhelmed by uncoupling, then mitochondrial membrane potential is not expected to be compromised.

Referring to Table 2: Identifiers, Calculated Physicochemical Properties, and Summary of Uncoupling Activity of Compounds Screened in Isolated Rat Liver Mitochondria. Notes to Table 2: pKa₍₁₎: acid dissociation constant of the most ionizable group; P: octanol-water partition coefficient of a compound in its unionized state; (1) nitro group oxygens out of plane; (2) methoxy group carbon out of plane; (3) 90° axial rotation of ring systems; (4) 45° axial rotation of ring systems; (5) isopropyl group carbons out of plane; pKa₍₁₎, log P_{octanol-water}, and three-dimensional structure assessed using ChemAxon Marvin 5.2: Uncoupling defined as increase in rate of basal oxygen consumption (state 4 respiration) per mg mitochondrial protein: Uncoupling expressed as % of average rate of ADP-stimulated consumption (state 3 respiration) per mg mitochondrial protein measured in vehicle-treated mitochondria of the same preparation. Functional capacity defined as the difference between rate of ADP-stimulated oxygen consumption and rate of basal oxygen consumption. Residual functional capacity defined as functional capacity expressed as % of average functional capacity in vehicle-treated mitochondria of the same preparation. Inhibitory effect estimated as decrease in functional capacity over and above that attributable to uncoupling. Oxygen consumption assays were performed in isolated rat liver mitochondria. Each compound tested in 2-3 independent preparations. Results are expressed as mean±SEM. 0.1% DMSO used as vehicle and to establish baseline functional measurements.

TABLE 2
Identifiers, Calculated Physicochemical Properties, and Summary of Uncoupling Activity
of Compounds Screened in Isolated Rat Liver Mitochondria.

| | | | Mol. | | | |
|---|---|---|---|---|---|---|
| | | | Mass | pKa | | 3-D |
| Compound | Class | CAS # | (Da) | (lowest) | logP | Structure |
| 2,4- | reference | 51- | 184.1 | 4.0 | 1.6 | planar (1) |
| dinitrophenol | | 28-5 | | | | |
| anthraquinone | anthraquinone | 84- | 208.2 | n/a | 2.9 | fully |
| | | 65-1 | | | | planar |
| frangulic acid | anthraquinone | 518- | 270.2 | 7.1 | 3.8 | fully |
| | | 82-1 | | | | planar |
| chalcone | chalconoid | 94- | 208.3 | n/a | 3.9 | fully |
| | | 41-7 | | | | planar |
| butein | chalconoid | 487- | 272.3 | 7.4 | 3.3 | fully |
| | | 52-5 | | | | planar |
| homobutein | chalconoid | 34000- | 286.3 | 7.4 | 3.5 | planar (2) |
| | | 39-0 | | | | |
| 4′-hydroxy- | chalconoid | 2657- | 224.3 | 7.9 | 3.6 | fully |
| chalcone | | 25-2 | | | | planar |
| isoliquiritigenin | chalconoid | 961- | 256.3 | 7.4 | 3.6 | fully |
| | | 29-5 | | | | planar |
| phloretin | (dihydro) | 60- | 274.3 | 7.0 | 3.9 | planar (3) |
| | halconoid | 82-2 | | | | |
| caffeic acid | cinnamate | 331- | 180.2 | 3.6 | 1.5 | fully |
| | | 39-5 | | | | planar |
| caffeic acid | cinnamate | 104594- | 284.3 | 9.2 | 3.9 | planar (3) |
| phenethyl ester | | 70-9 | | | | |
| ferulic acid | cinnamate | 1135- | 194.2 | 3.8 | 1.7 | planar (2) |
| | | 24-6 | | | | |
| curcumin | cinnamate/diarylheptanoid | 458- | 368.4 | 9.1 | 4.1 | non- |
| | | 37-7 | | (keto) | (keto) | planar |
| | | | | | | (keto) |
| | | | | 7.9 | 3.8 | planar |
| | | | | (enol) | (enol) | (enol)(2) |
| amentoflavone | bis-flavonoid | 1617- | 538.5 | 6.8 | 5.1 | non- |
| | | 53-4 | | | | planar |
| cupressuflavone | bis-flavonoid | 3952- | 538.5 | 6.5 | 5.1 | non- |
| | | 18-9 | | | | planar |
| sciadopitysin | bis-flavonoid | 521- | 580.5 | 6.9 | 5.5 | non- |
| | | 34-6 | | | | planar |
| (+) catechin | flavanol | 154- | 290.3 | 8.6 | 1.8 | non- |
| | | 23-4 | | | | planar |
| (−) | flavanol | 989- | 458.4 | 7.8 | 3.1 | non- |
| epigallocatechin | | 51-5 | | | | planar |
| gallate | | | | | | |
| (±) hesperetin | flavanone | 6909 | 302.3 | 7.3 | 2.7 | planar |
| | | 7-99-0 | | | | (2; 4)(stereoisomer |
| | | | | | | A) |
| | | | | | | non- |
| | | | | | | planar |
| | | | | | | (stereoisomer |
| | | | | | | B) |
| (±) naringenin | flavanone | 480- | 272.3 | 7.3 | 2.8 | planar |
| | | 40-1 | | | | (4)(stereoisomer |
| | | | | | | A) |
| | | | | | | non- |
| | | | | | | planar |
| | | | | | | (stereoisomer |
| | | | | | | B) |
| silibinin (A and | flavanone | 2288 | 482.4 | 7.2 | 2.6 | non- |
| B) | | 8-70-6 | | | | planar |
| datiscetin | flavonol | 480- | 286.2 | 4.5 | 2.5 | planar (4) |
| | | 15-9 | | | | |
| galangin | flavonol | 548- | 270.2 | 4.8 | 2.8 | planar (4) |
| | | 83-4 | | | | |
| kaempferol | flavonol | 520- | 286.2 | 4.7 | 2.5 | planar (4) |
| | | 18-3 | | | | |
| morin | flavonol | 480- | 302.2 | 4.5 | 1.5 | planar (4) |
| | | 16-0 | | | | |
| myricetin | flavonol | 529- | 318.2 | 4.4 | 1.9 | planar (4) |
| | | 44-2 | | | | |
| quercetin | flavonol | 117- | 302.2 | 4.6 | 2.2 | planar (4) |
| | | 39-5 | | | | |
| flavone | flavone/isoflavone | 525- | 222.2 | n/a | 3.0 | planar (4) |
| | | 82-6 | | | | |
| apigenin | flavone/isoflavone | 520- | 270.2 | 7.3 | 2.7 | planar (4) |
| | | 36-5 | | | | |
| biochanin A | flavone/isoflavone | 491- | 284.3 | 7.3 | 3.2 | planar |
| | | 80-5 | | | | (2; 4) |
| chrysin | flavone/isoflavone | 480- | 254.2 | 7.3 | 3.0 | planar (4) |
| | | 40-0 | | | | |
| daidzein | flavone/isoflavone | 486- | 254.2 | 7.5 | 2.7 | planar (4) |
| | | 66-8 | | | | |
| formononetin | flavone/isoflavone | 485- | 268.3 | 7.5 | 2.9 | planar |
| | | 72-3 | | | | (2; 4) |
| genistein | flavone/isoflavone | 446- | 270.2 | 7.3 | 3.1 | planar (4) |
| | | 72-0 | | | | |
| phenol | simple | 108- | 94.1 | 10.0 | 1.7 | fully |
| | phenolic | 95-2 | | | | planar |
| benzoic acid | simple | 65- | 122.1 | 4.1 | 1.6 | fully |
| | phenolic | 85-0 | | | | planar |
| carvacrol | simple | 499- | 150.2 | 10.4 | 3.4 | planar (5) |
| | phenolic | 75-2 | | | | |
| catechol | simple | 120- | 110.1 | 9.3 | 1.4 | fully |
| | phenolic | 80-9 | | | | planar |
| gallic acid | simple | 149- | 170.1 | 3.9 | 0.7 | fully |
| | phenolic | 91-7 | | | | planar |
| hydroquinone | simple | 123- | 110.1 | 9.7 | 1.4 | fully |
| | phenolic | 31-9 | | | | planar |
| 4-acetylphenol | simple | 99- | 136.2 | 7.8 | 1.2 | fully |
| | phenolic | 93-4 | | | | planar |
| 7-hydroxy | simple | 5988 | 162.1 | 7.5 | 1.4 | fully |
| chromone | phenolic | 7-89-7 | | | | planar |
| pyrogallol | simple | 87- | 126.1 | 8.8 | 1.1 | fully |
| | phenolic | 66-1 | | | | planar |
| resorcinol | simple | 108- | 110.1 | 8.9 | 1.4 | fully |
| | phenolic | 46-3 | | | | planar |
| salicylic acid | simple | 69- | 138.1 | 2.8 | 2.0 | fully |
| | phenolic | 72-7 | | | | planar |
| thymol | simple | 89- | 150.2 | 10.6 | 3.4 | planar (5) |
| | phenolic | 83-8 | | | | |
| vanillin | simple | 121- | 152.2 | 7.8 | 1.2 | planar (2) |
| | phenolic | 33-5 | | | | |
| stilbene (trans) | stilbenoid | 103- | 180.3 | n/a | 4.3 | fully |
| | | 30-0 | | | | planar |
| piceatannol | stilbenoid | 4339- | 244.2 | 8.7 | 3.1 | fully |
| (trans) | | 71-3 | | | | planar |
| pinosylvin | stilbenoid | 102- | 212.2 | 8.8 | 3.7 | fully |
| (trans) | | 61-4 | | | | planar |
| resveratrol | stilbenoid | 501- | 228.2 | 8.7 | 3.4 | fully |
| (trans) | | 36-0 | | | | planar |

| | Test | | Residual | Inhibitory | |
|---|---|---|---|---|---|
| | conc. | Uncoupling | capacity | effect | |
| Compound | (μM) | (%) | (%) | (%) | Notes |
| 2,4- | 100 | 143 ± 5 | 0 ± 0 | 0 | physicochemical |
| dinitrophenol | | | | | properties altered |
| | | | | | by in-situ |
| | | | | | dimerization [23B] |
| | 25 | 127 ± 11 | 14 ± 6 | 0 | |
| anthraquinone | 100 | −1 ± 1 | 85 ± 21 | 15 | |
| frangulic acid | 100 | 78 ± 14 | 0 ± 0 | 22 | reported uncoupling |
| | | | | | activity of related |
| | | | | | compounds in plant |
| | | | | | mitochondria [67B] |
| | 25 | 59 ± 16 | 0 ± 0 | 41 | |
| chalcone | 100 | −6 ± 0 | 35 ± 5 | 65 | |
| butein | 100 | 71 ± 4 | 0 ± 0 | 29 | reported uncoupler |
| | | | | | in plant |
| | | | | | mitochondria [68B] |
| | 25 | 26 ± 4 | 51 ± 12 | 23 | reported inhibitor of |
| | | | | | complex II [83B] |
| homobutein | 100 | 56 ± 5 | 22 ± 3 | 22 | |
| | 25 | 6 ± 0 | 79 ± 7 | 15 | |
| 4′-hydroxy- | 100 | 74 ± 14 | 26 ± 19 | 0 | reported uncoupler |
| chalcone | | | | | [69B] |
| | 25 | 13 ± 2 | 80 ± 2 | 7 | |
| isoliquiritigenin | 100 | 108 ± 7 | 0 ± 0 | 0 | reported uncoupler |
| | | | | | in plant |
| | | | | | mitochondria [68B] |
| | 25 | 24 ± 3 | 65 ± 6 | 11 | |
| phloretin | 100 | 15 ± 3 | 41 ± 17 | 44 | reported inhibitor of |
| | | | | | ATP synthase [80B] |
| | 25 | 3 ± 1 | 99 ± 12 | 0 | |
| caffeic acid | 100 | 0 ± 1 | 79 ± 9 | 21 | |
| caffeic acid | 100 | 57 ± 35 | 0 ± 0 | 43 | reported uncoupler |
| phenethyl ester | | | | | with atypical |
| | | | | | structure-activity |
| | | | | | relationship [37B] |
| | 25 | 56 ± 13 | 39 ± 12 | 5 | |
| ferulic acid | 100 | −1 ± 1 | 91 ± 9 | 9 | |
| curcumin | 100 | 79 ± 6 | 0 ± 0 | 21 | reported inhibitor of |
| | | | | | ATP synthase [80B] |
| | 25 | 42 ± 8 | 41 ± 9 | 17 | |
| amentoflavone | 100 | 4 ± 1 | 39 ± 5 | 57 | |
| | 25 | 3 ± 0 | 70 ± 0 | 27 | |
| cupressuflavone | 100 | −2 ± 0 | 95 ± 4 | 5 | |
| sciadopitysin | 100 | −1 ± 0 | 98 ± 0 | 2 | |
| (+) catechin | 100 | −2 ± 0 | 98 ± 5 | 2 | |
| (−) | 100 | 0 ± 1 | 98 ± 2 | 2 | reported inhibitor of |
| epigallocatechin | | | | | ATP synthase [80B] |
| gallate | | | | | |
| (±) hesperetin | 100 | 5 ± 1 | 81 ± 2 | 14 | |
| | 25 | 1 ± 1 | 91 ± 8 | 8 | |
| (±) naringenin | 100 | 4 ± 2 | 65 ± 7 | 31 | |
| silibinin (A and | 100 | 5 ± 1 | 22 ± 5 | 73 | |
| B) | | | | | |
| datiscetin | 100 | 33 ± 3 | 0 ± 0 | 67 | |
| | 25 | 15 ± 2 | 52 ± 9 | 33 | |
| galangin | 100 | 95 ± 9 | 0 ± 0 | 5 | reported uncoupler |
| | | | | | [73B] |
| | 25 | 102 ± 3 | 2 ± 2 | 0 | |
| kaempferol | 100 | 25 ± 2 | 28 ± 4 | 47 | reported inhibitor of |
| | | | | | ATP synthase [80B] |
| | 25 | 10 ± 1 | 62 ± 5 | 28 | |
| morin | 100 | 1 ± 0 | 39 ± 3 | 60 | reported inhibitor of |
| | | | | | ATP synthase [80B] |
| myricetin | 100 | 11 ± 0 | 46 ± 3 | 43 | reported inhibitor of |
| | | | | | complex II [83B] |
| | 25 | 6 ± 3 | 66 ± 2 | 28 | |
| quercetin | 100 | 8 ± 1 | 12 ± 5 | 80 | reported uncoupler |
| | | | | | [74B] |
| | 25 | 4 ± 0 | 60 ± 10 | 36 | reported inhibitor of |
| | | | | | ATP synthase [79B; |
| | | | | | 80B; 73B; 81B; 20B] |
| flavone | 100 | −1 ± 2 | 75 ± 13 | 25 | reported inhibitor of |
| | | | | | complex I in plant |
| | | | | | mitochondria [82B] |
| apigenin | 100 | 19 ± 3 | 37 ± 8 | 44 | reported inhibitor of |
| | | | | | ATP synthase [80B] |
| | 25 | 9 ± 1 | 63 ± 9 | 28 | |
| biochanin A | 100 | 103 ± 22 | 0 ± 0 | 0 | reported inhibitor of |
| | | | | | ATP synthase [80B] |
| | 25 | 75 ± 36 | 27 ± 27 | 0 | |
| chrysin | 100 | 137 ± 16 | 0 ± 0 | 0 | reported uncoupler |
| | | | | | in vesicle system |
| | | | | | [71B] |
| | 25 | 109 ± 22 | 0 ± 0 | 0 | |
| daidzein | 100 | 6 ± 1 | 68 ± 4 | 26 | reported inhibitor of |
| | | | | | ATP synthase [80B] |
| | 25 | 3 ± 1 | 78 ± 5 | 19 | |
| formononetin | 100 | 24 ± 1 | 58 ± 8 | 18 | |
| | 25 | 7 ± 0 | 80 ± 5 | 13 | |
| genistein | 100 | 74 ± 4 | 20 ± 0 | 6 | reported inhibitor of |
| | | | | | ATP synthase [80B] |
| | 25 | 8 ± 0 | 77 ± 1 | 15 | |
| phenol | 100 | 0 ± 0 | 96 ± 4 | 4 | para-halogenated |
| | | | | | phenols reported |
| | | | | | uncouplers [70B] |
| benzoic acid | 100 | 0 ± 1 | 96 ± 3 | 4 | |
| carvacrol | 100 | 7 ± 2 | 83 ± 2 | 10 | reported uncoupler |
| | | | | | [35B] |
| catechol | 100 | 0 ± 0 | 88 ± 1 | 12 | reported inhibitor of |
| | | | | | oxidative |
| | | | | | phosphorylation |
| | | | | | [84B] |
| gallic acid | 100 | −2 ± 1 | 98 ± 1 | 2 | |
| hydroquinone | 100 | 0 ± 1 | 90 ± 8 | 10 | |
| 4-acetylphenol | 100 | 0 ± 0 | 87 ± 1 | 13 | |
| 7-hydroxy | 100 | 0 ± 0 | 86 ± 2 | 14 | |
| chromone | | | | | |
| pyrogallol | 100 | 1 ± 1 | 83 ± 17 | 16 | |
| resorcinol | 100 | 0 ± 0 | 91 ± 3 | 9 | |
| salicylic acid | 100 | 4 ± 0 | 90 ± 2 | 6 | reported uncoupler |
| | | | | | in plant |
| | | | | | mitochondria [72B]; |
| | | | | | physicochemical |
| | | | | | properties may be |
| | | | | | altered by in-situ |
| | | | | | dimerization |
| thymol | 100 | 6 ± 0 | 65 ± 5 | 29 | reported uncoupler |
| | | | | | [35B] |
| vanillin | 100 | 0 ± 1 | 74 ± 16 | 26 | |
| stilbene (trans) | 100 | −1 ± 1 | 99 ± 3 | 1 | |
| piceatannol | 100 | 5 ± 1 | 6 ± 3 | 89 | reported inhibitor of |
| (trans) | | | | | ATP synthase [80B, |
| | | | | | 81B] |
| | 25 | 1 ± 1 | 35 ± 7 | 64 | |
| pinosylvin | 100 | 3 ± 3 | 63 ± 4 | 34 | |
| (trans) | | | | | |
| | 25 | 0 ± 1 | 80 ± 13 | 20 | |
| resveratrol | 100 | 2 ± 0 | 36 ± 4 | 62 | reported inhibitor of |
| (trans) | | | | | ATP synthase [80B, |
| | | | | | 81B] |
| | 25 | 0 ± 0 | 71 ± 9 | 29 | |

Referring to Documents: [1B] N. Wollen, et al., Diabète Métab. 14 (1988) 88-91. [2B] A. Klip, et al., Diab. Care 13 (1990) 696-704. [3B] N. F. Wiernsperger, et al. Drugs 58 (Suppl. 1) (1999) 31-39. [4B] T. Teranishi, et al., Metab. Clin. Exp. 56 (2007) 1418-1424. [5B] Y. Tamura, et al., Diab. Obes. Metab. 10 (2008) 733-738. [6B] J. M. Lenhard et al., Biochem. Pharmacol. 54 (1997) 801-808. [7B] M. Suwa, et al., J. Appl. Physiol. 101 (2006) 1685-1692. [8B] D. G. Hardie, Int. J. Obes. 32 (Suppl. 4) (2008) S7-S12. [9B] W. W. Winder, J. Appl. Physiol. 91 (2001) 1017-1028. [10B] W. W. Winder, et al., Cell Biochem. Biophys. 47 (2007) 332-347. [11B] P. Misra, Expert Opin. Ther. Targets 12 (2008) 91-100. [12B] B. Viollet et al., Front. Biosci. 14 (2009) 3380-3400. [13B] G. R. Steinberg, et al., Physiol. Rev. 89 (2009) 1025-1078. [14B] M. Y. El-Mir, et al., J. Biol. Chem. 275 (2000) 223-228. [15B] M. R. Owen, et al., Biochem. J. 348 (Pt 3) (2000) 607-614. [16B] T. Hayashi, et al., Diabetes 49 (2000) 527-531. [17B] N. Turner, et al., Diabetes 57 (2008) 1414-1418. [18B] M. Jing, F. et al. Am. J. Physiol. 292 (2007) C477-C487. [19B] C. E. Park, et al., Exp. Mol. Med. 39 (2007) 222-229. [20B] H. M. Eid, et al., Mol. Nutr. Food Res. 54 (2010) 991-1003. [21B] V. Pirags, et al., Diabetologia 53 (2010) S354. [22B] B. Brunmair, et al., Diabetes 53 (2004) 1052-1059. [23B] J. Radziuk, et al., Curr. Drug Targets 3 (2003) 151-169. [24B] P. Jezek, et al., Int. J. Biochem. Cell Biol. 37 (2005) 2478-2503. [25B] S. Nakamura, et al., J. Biol. Chem. 284 (2009) 14809-14818. [26B] J. A. Chavez, et al., J. Biol. Chem. 278 (2003) 10297-10303. [27B] K. F. Petersen, et al., Am. J. Med. 119 (2006) S10-S16. [28B] S. Timmers, et al., Physiol. Behav. 94 (2008) 242-251. [29B] E. J. Anderson, et al., J. Clin. Invest. 119 (2009) 573-581. [30B] V. P. Skulachev, Biochim. Biophys. Acta 1363 (1998) 100-124. [31B] K. B. Wallace, et al. Ann. Rev. Pharmacol. Toxicol. 40 (2000) 353-388. [32B] H. Terada, Environ. Health Perspect. 87 (1990) 213-218. [33B] W. G. Whittingham, Uncouplers of oxidative phosphorylation, in: W. Kramer, U. Schirmer (Eds.), Modern crop protection compounds, Wiley, Weinheim, 2007, pp. 505-528. [34B] L. C. Martineau, et al., J. Ethnopharmacol. 127 (2010) 396-406. [35B] A. Benhaddou-Andaloussi, et al., Diab. Obes. Metab. 12 (2010) 148-157. [36B] A. Benhaddou-Andaloussi, et al., Evid. Based Compl. Alt. Med. (2011) 538671. [37B] H. M. Eid, et al., Biochem. Pharmacol. 79 (2010) 444-454. [38B] D. Johnson, H. A. Lardy, Isolation of liver or kidney mitochondria, in: R. W. Eastbrook, M. E. Pullman (Eds.), Methods in Enzymology, 10, Academic Press, New York, N.Y., 1967. [39B] M. Heishi, et al., Diabetologia 49 (2006) 1647-1655. [40B] C. H. Lau, et al., Phytother. Res. 22 (2008) 1384-1388. [41B] K. Yu, et al., Comp. Biochem. Physiol. C Toxicol. Pharmacol. 143 (2006) 225-231. [42B] F. H. Ziel, et al., Diabetes 37 (1988) 885-890. [43B] J. M. Ren, et al., J. Biol. Chem. 268 (1993) 16113-16115. [44B] L. Chang, et al., Mol. Med. 10 (2004) 65-71. [45B] N. Musi, et al., Acta. Physiol. Scandinav. 178 (2003) 337-345. [46B] S. L. McGee, et al., Clin. Exp. Pharmacol. Physiol. 33 (2006) 395-399. [47B] S. L. McGee, et al., Front. Biosci. 13 (2008) 3022-3033. [48B] M. Shetty, et al., Am. J. Physiol. 262 (1992) C527-0532. [49B] N. Bashan, et al., Am. J. Physiol. 264 (1993) C430-C440. [50B] T. Tsakiridis, et al., Biochem. J. 309 (Pt 1) (1995) 1-5. [51B] M. Shetty, et al., Am. J. Physiol. 265 (1993) C1224-C1229. [52B] N. Matsuura, et al., Can. J. Biochem. 53 (1975) 28-36. [53B] B. Christ, et al., Biochem. J. 238 (1986) 185-191. [54B] E. van Schaftingen, et al., Biochem. J. 362 (2002) 513-532. [55B] N. Yabaluri, et al., J. Biosci. 35 (2010) 473-484. [56B] P. A. Lochhead, et al., Diabetes 49 (2000) 896-903. [57B] A. Barthel, et al., Endocrinology 143 (2002) 3183-3186. [58B] K. Morioka, et al., Int. J. Mol. Med. 15 (2005) 449-452. [59B] R. S. Hundal, et al., Diabetes 49 (2000) 2063-2069. [60B] G. Zhou, et al., J. Clin. Invest. 108 (2001) 1167-1174. [61B] A. H. Remels, et al., Mol. Cell. Endocrinol. 315 (2009) 113-120. [62B] E. Barbieri, et al., J. Aging Res. (2011) (Article ID 845379). [63B] B. Chance, et al., J. Biol. Chem. 238 (1963) 439-444. [64B] G. Prezioso, et al., J. Bioenerg. 3 (1972) 377-385. [65B] D. G. Hammond, et al., J. Pharmacol. Exp. Ther. 293 (2000) 822-828. [66B] P. H. Lou, et al., Biochem. J. 407 (2007) 129-140. [67B] E. H. C. Verhaeren, Phytochemistry 19 (1980) 501-503. [68B] P. Ravanel, et al., Phytochemistry 21 (1982) 2845-2850. [69B] B. Inoue, et al., J. Toxicol. Sci. 7 (1982) 245-254. [70B] F. Izushi, et al., Acta Med. Okayama 42 (1988) 7-14. [71B] C. van Dijk, et al., Biochem. Pharmacol. 60 (2000) 1593-1600. [72B] C. Norman, et al., Plant Physiol. 134 (2004) 492-501. [73B] D. J. Dorta, et al., Chem. Biol. Interact. 152 (2005) 67-78. [74B] S. Trumbeckaite, et al., Biomed. Pharmacother. 60 (2006) 245-248. [75B] H. Ikuma, et al., Plant Physiol. 42 (1967) 1400-1406. [76B] G. Galati, et al., Drug Metab. Drug Interact. 17 (2000) 311-349. [77B] D. Morin, et al., FEBS Lett. 495 (2001) 131-136. [78B] M. Salvi, et al., Biochim. Biophys. Acta 1556 (2002) 187-196. [79B] D. R. Lang, et al. Biochim. Biophys. Acta 333 (1974) 180-186. [80B] J. Zheng, et al. Br. J. Pharmacol. 130 (2000) 1115-1123. [81B] J. R. Gledhill, et al., Proc. Natl. Acad. Sci. 104 (2007) 13632-13637. [82B] P. Ravanel, Phytochemistry 20 (1981) 2101-2103. [83B]W. F. Hodnick, et al., Biochem. Pharmacol. 35 (1986) 2345-2357. [84B] G. E. Barreto, et al., Acta Cir. Bras. (Suppl. 1)(2005) 72-77. [85B] E. S. Lee, et al., Biochem. Biophys. Res. Comm. 361 (2007) 854-858. [86B] K. A. Rasbach, et al., J. Pharmacol. Exp. Ther. 325 (2008) 536-543. [87B] M. S. Lee, et al., Biol. Pharm. Bull. 32 (2009) 504-508. [88B] D. Govorko, et al., Am. J. Physiol. 293 (2007) E1503-E1510. [89B] M. S. Choi, et al., Diab. Metab. Res. Rev. 24 (2008) 74-81. [90B] B. Guigas, et al. Cell. Physiol. Biochem. 20 (2007) 925-934. [91B] Q. F. Collins, et al., J. Biol. Chem. 282 (2007) 30143-30149. [92B] A. Purushotham, et al., Mol. Nutr. Food Res. 53 (2009) 300-307. [93B] H. Ligeret, et al., Free Radic. Biol. Med. 36 (2004) 919-929. [94B] M. E. Young, et al., FEBS Lett. 382 (1996) 43-47. [95B] H. Jamal, et al., Biol. Med, 1 (2009) 107-115.

One of ordinary skill in the art would readily appreciate that the pharmaceutical formulations and methods described herein can be prepared and practiced by applying known procedures in the pharmaceutical arts. These include, for example, unless otherwise indicated, conventional techniques of pharmaceutical sciences including pharmaceutical dosage form design, drug development, pharmacology, of organic chemistry, and polymer sciences. See generally, for example, Remington: The Science and Practice of Pharmacy, 21^{st} edition, Lippincott, Williams & Wilkins, (2005).

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

The referenced items are provided solely for their disclosure prior to the filing date of the present application. Nothing herein is to be construed as an admission that the present invention is not entitled to antedate such material by virtue of prior invention.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Unless otherwise indicated, the words and phrases presented in this document have their ordinary meanings to one of skill in the art. Such ordinary meanings can be obtained by reference to their use in the art and by reference to general and scientific dictionaries, for example, Webster's Third New International Dictionary, Merriam-Webster Inc., Springfield, Mass., 1993, The American Heritage Dictionary of the English Language, Houghton Mifflin, Boston Mass., 1981, and Hawley's Condensed Chemical Dictionary, 14^{th} edition, Wiley Europe, 2002.

The following explanations of certain terms are meant to be illustrative rather than exhaustive. These terms have their ordinary meanings given by usage in the art and in addition include the following explanations.

As used herein, the term “about” refers to a variation of 10 percent of the value specified; for example about 50 percent carries a variation from 45 to 55 percent.

As used herein, the term “and/or” refers to any one of the items, any combination of the items, or all of the items with which this term is associated.

As used herein, the singular forms “a,” “an,” and “the” include plural reference unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation.

Specific and preferred values listed below for radicals, substituents, and ranges, are for illustration only; they do not exclude other defined values or other values within defined ranges for the radicals and substituents.

As used herein, a dash (“-”) that is not between two letters or symbols is used to indicate a point of attachment for a moiety or substituent. For example, the moiety —CONH₂ is attached through the carbon atom.

As used herein, the terms “a.u.” or “a.u. of power” refer to arbitrary units of predicted rate of energy dissipation by a protonophore.

As used herein, the term “acidity-modulating substituent” refers to an electron-withdrawing or electron-donating substituent or group positioned in such way as to alter the acid-dissociation behaviour of an ionizable substituent or group.

As used herein, the term “acyl” group refers to a group containing a carbonyl moiety wherein the group is bonded via the carbonyl carbon atom. The carbonyl carbon atom is also bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen atom, the group is a “formyl” group, an acyl group as the term is defined herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

As used herein, the term “administration” refers to a method of placing a device to a desired site. The placing of a device can be by any pharmaceutically accepted means such as by swallowing, retaining it within the mouth until the drug has been dispensed, placing it within the buccal cavity, inserting, implanting, attaching, etc. These and other methods of administration are known in the art.

As used herein, the term “amino” refers to —NH₂. The amino group can be optionally substituted as defined herein for the term “substituted.” The term “alkylamino” refers to —NR₂, wherein at least one R is alkyl and the second R is alkyl or hydrogen. The term “acylamino” refers to N(R)C(═O)R, wherein each R is independently hydrogen, alkyl, or aryl.

As used herein, the terms “amide” (or “amido”) refer to C- and N-amide groups, i.e., —C(O)NR₂, and —NRC(O)R groups, respectively. Amide groups therefore include but are not limited to carbamoyl groups (—C(O)NH₂) and formamide groups (—NHC(O)H).

As used herein, the term “alkanoyl” or “alkylcarbonyl” refers to —C(═O)R, wherein R is an alkyl group as previously defined.

As used herein, the term “acyloxy” or “alkylcarboxy” refers to —O—C(═O)R, wherein R is an alkyl group as previously defined. Examples of acyloxy groups include, but are not limited to, acetoxy, propanoyloxy, butanoyloxy, and pentanoyloxy. Any alkyl group as defined above can be used to form an acyloxy group.

As used herein, the term “alkoxycarbonyl” refers to —C(═O)OR (or “COOR”), wherein R is an alkyl group as previously defined.

As used herein, the term “alkyl” refers to a C₁-C₁₈ hydrocarbon containing normal, secondary, tertiary or cyclic carbon atoms. Examples are methyl, ethyl, 1-propyl, 2-propyl, 1-butyl, 2-methyl-1-propyl (iso-butyl, —CH₂CH(CH₃)₂), 2-butyl (sec-butyl, —CH(CH₃)CH₂CH₃), 2-methyl-2-propyl (tert-butyl, —C(CH₃)₃), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl.

The alkyl can be a monovalent hydrocarbon radical, as described and exemplified above, or it can be a divalent hydrocarbon radical (i.e., alkylene).

As used herein, the term “alkenyl” refers to a C₂-C₁₈ hydrocarbon containing normal, secondary, tertiary or cyclic carbon atoms with at least one site of unsaturation, i.e., a carbon-carbon, sp² double bond. Examples include, but are not limited to: ethylene or vinyl (—CH═CH₂), allyl (—CH₂CH═CH₂), cyclopentenyl (—C₅H₇), and 5-hexenyl (—CH₂CH₂CH₂CH₂CH═CH₂). The alkenyl can be a movalent hydrocarbon radical, as described and exemplified above, or it can be a divalent hydrocarbon radical (i.e., alkenylene).

As used herein, the term “alkylene” refers to a saturated, branched or straight chain or cyclic hydrocarbon radical of 1-18 carbon atoms, and having two monovalent radical centers derived by the removal of two hydrogen atoms from the same or different carbon atoms of a parent alkane. Typical alkylene radicals include, but are not limited to: methylene (—CH₂—) 1,2-ethylene (—CH₂CH₂—), 1,3-propylene (—CH₂CH₂CH₂—), 1,4-butylene (—CH₂CH₂CH₂CH₂—), and the like.

As used herein, the term “alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethenylene (—CH═CH—).

As used herein, the term “alkoxy” refers to the group alkyl-O—, where alkyl is defined herein. Preferred alkoxy groups include, e.g., methoxy, ethoxy, n-propoxy, iso-propoxy, n-butoxy, tert-butoxy, sec-butoxy, n-pentoxy, n-hexoxy, 1,2-dimethylbutoxy, and the like.

As used herein, the term “alcohol” refers to a compound of a general formula ROH.

As used herein, the term “AMPK” refers to the AMP-activated protein kinase.

As used herein, the term “aryl” or “aromatic” refers to an unsaturated aromatic carbocyclic group of from 6 to 30 carbon atoms having a single ring (e.g., phenyl) or multiple condensed (fused) rings, wherein at least one ring is aromatic (e.g., naphthyl, dihydrophenanthrenyl, fluorenyl, or anthryl). Preferred aryls include phenyl, naphthyl and the like. The aryl can optionally be a divalent radical, thereby providing an arylene.

As used herein, the terms “aryloxy” and “arylalkoxy” refer to, respectively, an aryl group bonded to an oxygen atom and an aralkyl group bonded to the oxygen atom at the alkyl moeity. Examples include but are not limited to phenoxy, naphthyloxy, and benzyloxy.

As used herein, the term “cancer” refers to any type of cancer, including skin cancer, lung cancer, pancreatic cancer, ovarian cancer, liver cancer, glioma, prostate cancer, colon cancer, breast cancer, endometrial cancer, leukemia, CNS cancer, melanoma, renal cancer, and the like.

As used herein, the term “carboxyl” refers to —COOH.

As used herein, the term “chemically feasible” refers to a bonding arrangement or a compound where the generally understood rules of organic structure are not violated; for example a structure within a definition of a claim that would contain in certain situations a pentavalent carbon atom that would not exist in nature would be understood to not be within the claim.

When a substituent is specified to be an atom or atoms of specified identity, “or a bond”, a configuration is referred to when the substituent is “a bond” that the groups that are immediately adjacent to the specified substituent are directly connected to each other by a chemically feasible bonding configuration.

All chiral, diastereomeric, racemic forms of a structure are intended, unless a particular stereochemistry or isomeric form is specifically indicated. Compounds used in the present invention can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

Although the structures of the compounds disclosed herein may be depicted as having one particicular configuration, for example, a double bond with a cis, the invention covers all possible configurations and/or permutations.

As used herein, the term “cell membrane (or plasma membrane or mitochondrial membrane)” refers to a semi-permeable lipid bilayer that has a common structure in all living cells; it contains primarily proteins and lipids that are involved in a myriad of important cellular processes.

As used herein, the phrase “compounds of the disclosure” refer to compounds of Formulas (I-V) and pharmaceutically acceptable enantiomers, diastereomers, and salts thereof. Similarly, references to intermediates, are meant to embrace their salts where the context so permits.

As used herein the term “diabetes,” includes both insulin-dependent diabetes mellitus (i.e., IDDM, also known as Type 1 diabetes) and non-insulin-dependent diabetes mellitus (i.e., NIDDM, also known as Type 2 diabetes) and is characterized by a fasting plasma glucose level of greater than or equal to 126 mg/dl.

As used herein, the term “an effective amount” refers to an amount sufficient to effect beneficial or desired results. An effective amount can be administered in one or more administrations, applications, or dosages. Determination of an effective amount for a given administration is well within the ordinary skill in the pharmaceutical arts.

As used herein, the term “energy transduction” refers to proton transfer through the respiratory complexes embedded in a membrane, utilizing electron transfer reactions to pump protons across the membrane and create an electrochemical potential also known as the proton electrochemical gradient.

As used herein the term “energy transformation” in cells refers to chemical bonds being constantly broken and created, to make the exchange and conversion of energy possible It is generally stated that that transformation of energy from a more to a less concentrated form is the driving force of all biological or chemical processes that are responsible for the respiration of a cells.

As used herein, the phrase “fused aromatic ring system” alone or in combination, refers to a carbocyclic aromatic ring radical fused to another carbocyclic aromatic ring radical, the two having two atoms in common. Typical fused aromatic ring systems include, but are not limited to napthalene, quinoline, isoquinoline, indole, and isoindole.

As used herein, the term “halo” refers to fluoro, chloro, bromo, and iodo. Similarly, the term “halogen” refers to fluorine, chlorine, bromine, and iodine.

As used herein, the term “haloalkyl” refers to alkyl as defined herein substituted by 1-4 halo groups as defined herein, which may be the same or different. Representative haloalkyl groups include, by way of example, trifluoromethyl, 3-fluorododecyl, 12,12,12-trifluorododecyl, 2-bromooctyl, 3-bromo-6-chloroheptyl, and the like.

As used herein, the term “heteroaryl” is defined herein as a monocyclic, bicyclic, or tricyclic ring system containing one, two, or three aromatic rings and containing at least one nitrogen, oxygen, or sulfur atom in an aromatic ring, and which can be unsubstituted or substituted. The heteroaryl can optionally be a divalent radical, thereby providing a heteroarylene.

Examples of heteroaryl groups include, but are not limited to, 2H-pyrrolyl, 3H-indolyl, 4H-quinolizinyl, 4nH-carbazolyl, acridinyl, benzo[b]thienyl, benzothiazolyl, p-carbolinyl, carbazolyl, chromenyl, cinnaolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, naptho[2,3-b], oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, and xanthenyl. In one embodiment the term “heteroaryl” denotes a monocyclic aromatic ring containing five or six ring atoms containing carbon and 1, 2, 3, or 4 heteroatoms independently selected from the group non-peroxide oxygen, sulfur, and N(Z) wherein Z is absent or is H, O, alkyl, phenyl, or benzyl. In another embodiment heteroaryl denotes an ortho-fused bicyclic heterocycle of about eight to ten ring atoms derived therefrom, particularly a benz-derivative or one derived by fusing a propylene, or tetramethylene diradical thereto.

As used herein, the term “heterocycle” or “heterocyclyl” refers to a saturated or partially unsaturated ring system, containing at least one heteroatom selected from the group oxygen, nitrogen, and sulfur, and optionally substituted with alkyl, or C(═O)OR^{b}, wherein Rb is hydrogen or alkyl. Typically heterocycle is a monocyclic, bicyclic, or tricyclic group containing one or more heteroatoms selected from the group oxygen, nitrogen, and sulfur. A heterocycle group also can contain an oxo group (═O) attached to the ring. Non-limiting examples of heterocycle groups include 1,3-dihydrobenzofuran, 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuclidine, and thiomorpholine. The heterocycle can optionally be a divalent radical, thereby providing a heterocyclene.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles.

As used herein, the term “herbicide” refers to a molecule or combination of molecules that inhibits or otherwise kills unwanted plants, such as, but not limited to, deleterious or annoying weeds, broadleaf plants, grasses and sedges and can be used for crop protection, edifice protection or turf protection.

As used herein, the term “insecticide” refers to the active chemical compound or ingredient, which kills or causes knockdown of insects.

As used herein, the terms “include,” “for example,” “such as,” and the like are used illustratively and are not intended to limit the present invention.

As used herein, the terms “individual,” “host,” “subject,” and “patient” are used interchangeably, and refer to a mammal, including, but not limited to, primates, including simians and humans.

As used herein, the term “ionized species” refers to the ionized states of a protic compound that exist at both a pH of a first side and a pH of a second side of a biological membrane across which the compound exerts protonophoric activity.

As used herein, the term “keto” refers to (C═O).

As to any of the groups described herein, which contain one or more substituents, it is understood, of course, that such groups do not contain any substitution or substitution patterns which are sterically impractical and/or synthetically non-feasible. In addition, the compounds of this disclosed subject matter include all stereochemical isomers arising from the substitution of these compounds.

Selected substituents within the compounds described herein are present to a recursive degree. In this context, “recursive substituent” means that a substituent may recite another instance of itself. Because of the recursive nature of such substituents, theoretically, a large number may be present in any given claim. One of ordinary skill in the art of medicinal chemistry and organic chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis.

Recursive substituents are an intended aspect of the disclosed subject matter. One of ordinary skill in the art of medicinal and organic chemistry understands the versatility of such substituents. To the degree that recursive substituents are present in an claim of the disclosed subject matter, the total number will be determined as set forth above.

As used herein, the term “mammal” refers to any of a class of warm-blooded higher vertebrates that nourish their young with milk secreted by mammary glands and have skin usually more or less covered with hair, and non-exclusively includes humans and non-human primates, their children, including neonates and adolescents, both male and female, livestock species, such as horses, cattle, sheep, and goats, and research and domestic species, including dogs, cats, mice, rats, guinea pigs, and rabbits.

As used herein, the phrase “minimal projection area” refers to the smallest two-dimensional molecular surface of a compound that can be projected from a pseudo three-dimensional rendering of that compound, preferably according to van der Waals's atomic radii, and preferably in the compound's lowest-energy state or in the state most likely to be assumed by the compound under physiological conditions.

As used herein, the term “mitochondria” refers to membrane-enclosed organelles, found in most eukaryotic cells (animal cells, plant cells, and fungi).

As used herein, the term “molecular weight” refers to a weight-average molecular weight, as is well known in the art.

As used herein, the term “obesity” refers to a condition in which there is an excess of body fat.

As used herein, the terms “optional” or “optionally” mean that the subsequently described event or condition may but need not occur, and that the description includes instances where the event or condition occurs and instances in which it does not. For example, “optionally substituted” means that the named substituent may be present but need not be present, and the description includes situations where the named substituent is included and situations where the named substituent is not included.

As used herein, the term “oxo” refers to =0.

As used herein, the term “patient” refers to a warm-blooded animal, and preferably a mammal, for example, a cat, dog, horse, cow, pig, mouse, rat, or primate, including a human.

As used herein, the term “permeability” refers to the rate of movement of the unionized species or of one of the ionized species (as specified) of a protic compound through the biological membrane across which the protic compound exerts protonophoric activity.

As used herein, the term “pest” refers to any insect, rodent, fish, nematode, fungus, weed, or any form of terrestrial or aquatic plant or animal life or virus, or bacterial organism or microorganisms (except those viruses, bacteria or other microorganisms existing in living humans or other living animals) considered injurious to health, the environment or man's economic well-being.

As used herein, the term “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio. Several pharmaceutically acceptable ingredients are known in the art and official publications such as The United States Pharmacoepia describe the analytical criteria to assess the pharmaceutical acceptability of numerous ingredients of interest.

As used herein, the term “pharmaceutically acceptable salts” refers to ionic compounds, wherein a parent non-ionic compound is modified by making acid or base salts thereof.

As used herein, the term “pharmacologically active agent” refers to a chemical compound, complex or composition that exhibits a desirable effect in the biological context, i.e., when administered to a subject. The term includes pharmacologically active, pharmaceutically acceptable derivatives of those active agents specifically mentioned herein, including, but not limited to, salts, esters, amides, prodrugs, active metabolites, isomers, analogs, crystalline forms, hydrates, and the like.

As used herein, the terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the terms “prevent,” “preventative,” “prevention,” “protect,” and “protection” refer to medical procedures that keep the malcondition from occurring in the first place. The terms mean that there is no or a lessened development of disease or disorder where none had previously occurred, or no further disorder or disease development if there had already been development of the disorder or disease.

As used herein, the term “prodrug” refers to any pharmaceutically acceptable form of compound of the Formulas (I)-(V), which, upon administration to a patient, provides a compound of the Formulas (I)-(V). Pharmaceutically acceptable prodrugs refer to a compound that is metabolized, for example hydrolyzed or oxidized, in the host to form a compound of the formula (I) or formula (II). Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. The prodrug can be readily prepared from the compounds of Formulas (I)-(V) using methods known in the art. See, e.g. See Notari, R. E., “Theory and Practice of Prodrug Kinetics,” Methods in Enzymology, 112:309 323 (1985); Bodor, N., “Novel Approaches in Prodrug Design,” Drugs of the Future, 6(3):165 182 (1981); and Bundgaard, H., “Design of Prodrugs: Bioreversible-Derivatives for Various Functional Groups and

Chemical Entities,” in Design of Prodrugs (H. Bundgaard, ed.), Elsevier, N.Y. (1985); Burger's Medicinal Chemistry and Drug Chemistry, 5th Ed., Vol. 1, pp. 172 178, 949 982 (1995). The prodrug may be prepared with the objective(s) of improved chemical stability, improved patient acceptance and compliance, improved bioavailability, prolonged duration of action, improved organ selectivity (including improved brain penetrance), improved formulation (e.g., increased hydrosolubility), and/or decreased side effects (e.g., toxicity). See e.g. T. Higuchi and V. Stella, “Prodrugs as Novel Delivery Systems”, Vol. 14 of the A.C.S. Symposium Series; Bioreversible Carriers in Drug Design, ed. Edward B. Roche, American Pharmaceutical Association and Pergamon Press, (1987). Prodrugs include, but are not limited to, compounds derived from compounds of Formulas (I)-(V) wherein hydroxy, amine or sulfhydryl groups, if present, are bonded to any group that, when administered to the subject, cleaves to form the free hydroxyl, amino or sulfhydryl group, respectively. Selected examples include, but are not limited to, biohydrolyzable amides and biohydrolyzable esters and biohydrolyzable carbamates, carbonates, acetate, formate and benzoate derivatives of alcohol and amine functional groups.

As used herein, the term “protonophore” refers to a compound that contains an acidic or basic chemical group that confers upon the compound acid-dissociation properties such that the compound is ionizable under physiological conditions and exists in both unionized and ionized states at both a pH of a first side and a pH of a second side of a biological membrane across which the compound exerts protonophoric activity.

As used herein, the term “protonophore” may be “mono-protic” wherein it is characterized by a single such acidic or basic chemical group, and exists in a single unionized state and a single ionized state at both the pH of the first side and the pH of the second side of the biological membrane across which the compound exerts protonophoric.

As used herein, the term “protonophore” may be “multi-protic” wherein it is characterized by multiple such acidic or basic groups, and exists in a single unionized state and in multiple different ionized states at both the pH of the first side and the pH of the second side of the biological membrane across which the compound exerts protonophoric activity.

As used herein, the term “reaction mixture” may contain all reagents for a particular reaction, or may lack at least one of the reagents for the reaction.

As used herein, the phrase “room temperature” refers to a temperature in the range of about 20° C. to about 30° C.

As used herein, the terms “stable compound” and “stable structure” are meant to indicate a compound that is sufficiently robust to survive isolation to a useful degree of purity from a reaction mixture, and formulation into an efficacious therapeutic agent. Only stable compounds are contemplated herein.

As used herein, the term “substituted” is intended to indicate that one or more hydrogens on the atom indicated in the expression using “substituted” is replaced with a selection from the indicated group(s), provided that the indicated atom's normal valency is not exceeded, and that the substitution results in a stable compound. Suitable indicated groups include, e.g., alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, acyloxy, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, carbamate, isocyanato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl, or hydroxy. When a substituent is oxo (i.e., ═O) or thioxo (i.e., ═S) group, then two hydrogens on the atom are replaced.

As used herein, the phrase “subject in need thereof” refers to a subject who is in need of treatment or prophylaxis as determined by a researcher, veterinarian, medical doctor or other clinician.

As used herein, the term “therapeutically effective amount” is intended to include an amount of a compound described herein, or an amount of the combination of compounds described herein, e.g., to treat or prevent the disease or disorder, or to treat the symptoms of the disease or disorder, in a host. As used herein, the terms “therapy,” and “therapeutic” refer to either “treatment” or “prevention,” thus, agents that either treat damage or prevent damage are “therapeutic.”

As used herein, the terms “treating” or “treat” or “treatment” refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease.

As used herein, the term “treatment” covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

As used herein, the term “U₅₀” refers to the concentration of a protonophore at which the protonophore induces 50% uncoupling in isolated mitochondria.

As used herein, the term “uncoupler” refers to a molecule or device that causes the separation of the energy stored in the proton electrochemical gradient (ΔμH÷) of membranes from the synthesis of ATP.

As used herein, the term “uncoupling” refers to the use of an uncoupler (a molecule or device) to cause the separation of the energy stored in the proton electrochemical gradient (ΔμH⁺) of membranes from the synthesis of ATP.

As used herein, the term “unionized species” refers to the unionized state of a protic compound that exists at both a pH of a first side and a pH of a second side of a biological membrane across which the compound exerts protonophoric activity.

As used herein, the phrase “z-length” refers to the molecular length of a compound measured perpendicularly to the plane of projection of the compound's minimal projection area defined herein.

In various embodiments, the compound or set of compounds, such as are used in the inventive methods, can be any one of any of the combinations and/or sub-combinations of the above-listed embodiments.

As used herein, “μg” denotes microgram, “mg” denotes milligram, “g” denotes gram, “μL” denotes microliter, “mL” denotes milliliter, “L” denotes liter, “nM” denotes nanomolar, “μM” denotes micromolar, “mM” denotes millimolar, “M” denotes molar, and “nm” denotes nanometer.

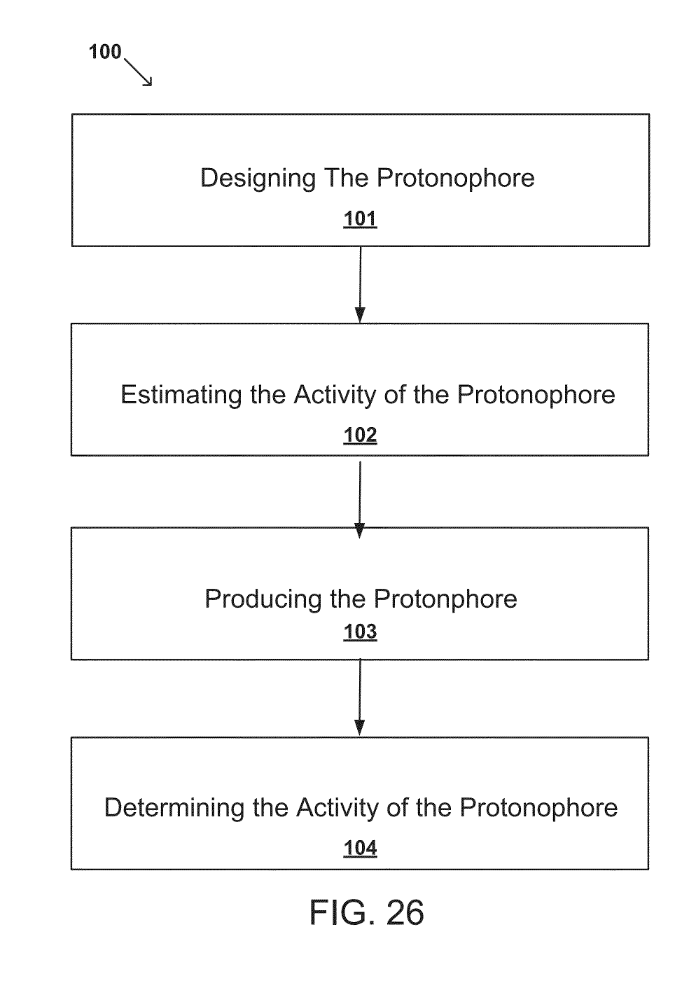

FIG. 26 is a block diagram illustrating an exemplary computer-assisted method of generating a protonophore 100. The method 100 includes designing the protonophore 101, estimating the activity of the protonophore 102, producing the protonophore 103, and determining the activity of the protonophore 104.

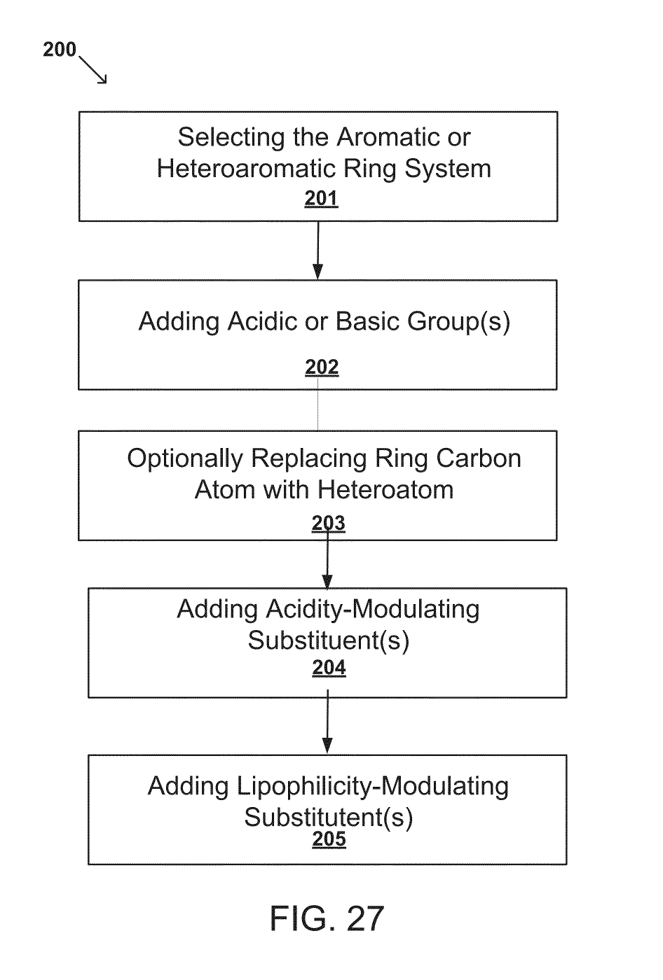

FIG. 27 is a block diagram illustrating an exemplary method of designing the protonophore 200. The method 200 includes selecting the aromatic or heteroaromatic ring system 201, adding one or more acidic groups 202, optionally replacing one or more of the ring atoms of the aromatic or heteroaromatic ring system with one or more unsubstituted acidic or basic nitrogen atoms 203, adding one or more acidity-modulating substituents 204, and adding one or more lipophilicity-conferring substituents 205.

### Methods of Making the Compounds of Formulas (I-V)

The compounds described herein can be prepared by any of the applicable techniques of organic synthesis. Many such techniques are well known in the art. However, many of the known techniques are elaborated in Compendium of Organic Synthetic Methods (John Wiley & Sons, New York) Vol. 1, Ian T. Harrison and Shuyen Harrison (1971); Vol. 2, Ian T. Harrison and Shuyen Harrison (1974); Vol. 3, Louis S. Hegedus and Leroy Wade (1977); Vol. 4, Leroy G. Wade Jr., (1980); Vol. 5, Leroy G. Wade Jr. (1984); and Vol. 6, Michael B. Smith; as well as March, J., Advanced Organic Chemistry, 4th Edition, John Wiley & Sons, New York (1992); Comprehensive Organic Synthesis. Selectivity, Strategy & Efficiency in Modern Organic Chemistry, In 9 Volumes, Barry M. Trost, Editor-in-Chief, Pergamon Press, New York (1993); Advanced Organic Chemistry, Part B: Reactions and Synthesis, 4th Ed.; Carey and Sundberg; Kluwer Academic/Plenum Publishers: New York (2001); Advanced Organic Chemistry, Reactions, Mechanisms, and Structure, 2nd Edition, March, McGraw Hill (1977); Protecting Groups in Organic Synthesis, 2nd Edition, Greene, T. W., and Wutz, P. G. M., John Wiley & Sons, New York (1991); Katritzky, A. R. Handbook of Heterocyclic Chemistry, Pergamon Press Ltd; New York (1985), Katritzky, A. R. Comprehensive Heterocyclic Chemistry, Volumes 1-8 Pergamon Press Ltd; New York (1984), and Comprehensive Organic Transformations, 2nd Edition, Larock, R. C., John Wiley & Sons, New York (1999). Exemplary methods of making the compounds described herein are described herein in the examples below.

Obviously, numerous modifications and variations of the presently disclosed subject matter are possible in light of the above teachings. It is therefore to be understood that within the scope of the claims, the disclosed subject matter may be practiced otherwise than as specifically described herein.

Specific ranges, values, and embodiments provided herein are for illustration purposes only and do not otherwise limit the scope of the disclosed subject matter, as defined by the claims.

The starting materials useful to synthesize the compounds of the present disclosure are known to those skilled in the art and can be readily manufactured or are commercially available.

The following methods set forth below are provided for illustrative purposes and are not intended to limit the scope of the claimed disclosure. It will be recognized that it may be necessary to prepare such a compound in which a functional group is protected using a conventional protecting group then to remove the protecting group to provide a compound of the present disclosure. The details concerning the use of protecting groups in accordance with the present disclosure are known to those skilled in the art.

The compounds of Formulas II to IV are listed in Tables 3 to 6, respectively.

TABLE 3

| Compound | | | |
|---|---|---|---|
| Number | Chemical Structure | Chemical Name | Comments |
| 1-1 | 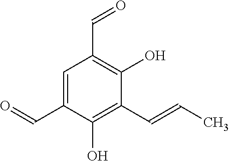 | 1,3-dihydroxy, 2-(propen- 1-yl), 3,6-diformyl, benzene | estimated pKa: 5.5; 7.2 estimated logP (neutral/ionized): 3.2/1.0; −1.2 estimated minimal projection area: 32 Å² estimated z-length: 11 Å predicted rate of energy dissipation: 515 × 10⁶ a.u. of power |
| 1-2 | 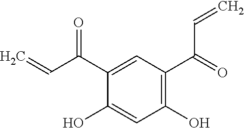 [SMILES: C=CC(=O)c1cc(C(=O)C=C)c(O)cc1O] | 1,3-dihydroxy, 4,6-di(prop- 2-en-1-one), benzene | estimated pKa: 6.0; 7.9 estimated logP (neutral/ionized): 3.3/1.1; −1.2 estimated minimal projection area: 33 Å² estimated z-length: 12 Å predicted rate of energy dissipation: 269 × 10⁶ a.u. of power |
| 1-3 | 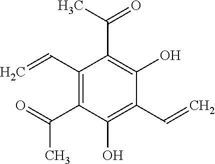 [SMILES: C=Cc1c(O)c(C(C)=O)c(C=C)c(C(C)=O)c1O] | 1,3 dihydroxy, 2,5- diethenyl, 4,6-diacetyl, benzene | estimated pKa: 5.9; 7.6 estimated logP (neutral/ionized): 3.3/1.0; −1.2 estimated minimal projection area: 43 Å² estimated z-length: 10 Å predicted rate of energy dissipation: 217 × 10⁶ a.u. of power |
| 1-4 | 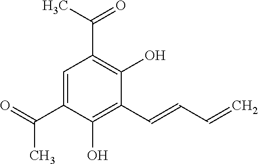 [SMILES: C=C/C=C/c1c(O)c(C(C)=O)cc(C(C)=O)c1O] | 1,3-dihydroxy, 2-((1E)- buta-1,3-dien-1-yl), 4,6- acetyl, benzene | estimated pKa: 5.9; 7.5 estimated logP (neutral/ionized): 3.1/0.8; −1.4 estimated minimal projection area: 34 Å² estimated z-length: 13 Å predicted rate of energy dissipation: 198 × 10⁶ a.u. of power |
| 2-1 | 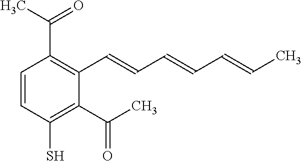 [SMILES: C/C=C/C=C/C=C/c1c(C(C)=O)ccc(S)c1C(C)=O] | 2,4-diacetyl, 3-((1E,3E,5E)- hepta-1,3,5-trien-1-yl), thiophenol | estimated pKa: 5.9 estimated logP (neutral/ionized): 3.4/1.9 estimated minimal projection area: 44 Å² estimated z-length: 15 Å predicted rate of energy dissipation: 151 × 10⁶ a.u. of power |
| 2-2 | 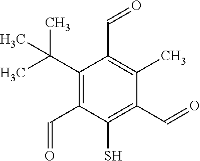 [SMILES: Cc1c(C=O)c(S)c(C=O)c(C(C)(C)C)c1C=O] | 2,4,6-triformyl, 3-methyl, 5-tert-butyl, thiophenol | estimated pKa: 5.7 estimated logP (neutral/ionized): 3.3/1.8 estimated minimal projection area: 43 Å² estimated z-length: 11 Å predicted rate of energy dissipation: 281 × 10⁶ a.u. of power |
| 2-3 | 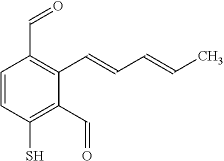 [SMILES: C/C=C/C=C/c1c(C=O)ccc(S)c1C=O] | 2,4-diformyl, 3-((1E,3E)- penta-1,3-dien-1-yl), thiophenol | estimated pKa: 5.8 estimated logP (neutral/ionized): 3.1/1.7 estimated minimal projection area: 30 Å² estimated z-length: 13 Å predicted rate of energy dissipation: 428 × 10⁶ a.u. of power |
| 2-4 | 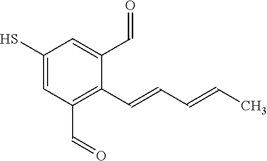 [SMILES: C/C=C/C=C/c1c(C=O)cc(S)cc1C=O] | 3,5-diformyl, 4-((1E,3E)- penta-1,3-dien-1-yl), thiophenol | estimated pKa: 5.9 estimated logP (neutral/ionized): 3.1/1.7 estimated minimal projection area: 32 Å² estimated z-length: 15 Å predicted rate of energy dissipation: 332 × 10⁶ a.u. of power |
| 2-5 | 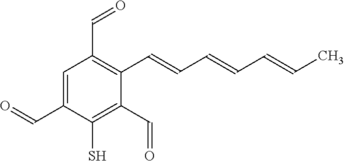 [SMILES: C/C=C/C=C/C=C/c1c(C=O)cc(C=O)c(S)c1C=O] | 2,4,6-triformyl, 3- ((1E,3E,5E)-hepta-1,3,5- trien-1-yl), thiophenol | estimated pKa: 5.5 estimated logP (neutral/ionized): 3.4/20 estimated minimal projection area: 32 Å² estimated z-length: 17 Å predicted rate of energy dissipation: 238 × 10⁶ a.u. of power |
| 2-6 | 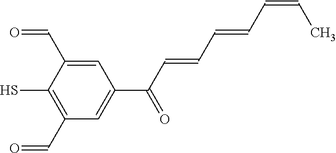 [SMILES: C\C=C/C=C/C=C/C(=O)c1cc(C=O)c(S)c(C=O)c1] | 2,6-diformyl, 4-((2E,4E,6E)- octa-2,4,6-trien-1-one), thiophenol | estimated pKa: 5.4 estimated logP (neutral/ionized): 3.3/1.8 estimated minimal projection area: 32 Å² estimated z-length: 17 Å predicted rate of energy dissipation: 348 × 10⁶ a.u. of power |
| 2-7 | 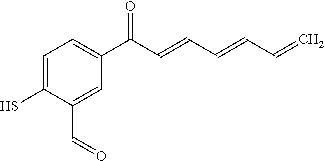 [SMILES: C=C/C=C/C=C/C(=O)c1ccc(S)c(C=O)c1] | 2-formyl, 4-((2E,4E,6E)- hepta-2,4,6-trien-1-one), thiophenol | estimated pKa: 5.7 estimated logP (neutral/ionized): 3.2/1.7 estimated minimal projection area: 26 Å² estimated z-length: 17 Å predicted rate of energy dissipation: 470 × 10⁶ a.u. of power |
| 2-8 | 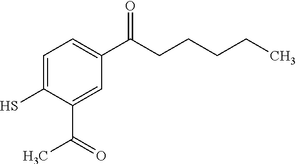 [SMILES: CCCCCC(=O)c1ccc(S)c(C(C)=O)c1] | 2-acetyl, 4-(hexan-1-one), thiophenol | estimated pKa: 5.8 estimated logP (neutral/ionized): 3.2/1.8 estimated minimal projection area: 33 Å² estimated z-length: 15 Å predicted rate of energy dissipation: 346 × 10⁶ a.u. of power |
| 3-1 | 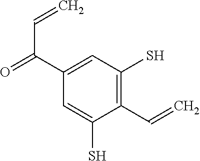 | 2-ethenyl, 3-sulfanyl, 5- (prop-2-en-1-one), thiophenol | estimated pKa: 5.7; 6.7 estimated logP (neutral/ionized): 3.2/1.8; 0.4 estimated minimal projection area: 32 Å² estimated z-length: 12 Å predicted rate of energy dissipation: 1084 × 10⁶ a.u. of power |
| 3-2 | 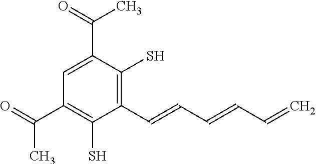 [SMILES: C=C/C=C/C=C/c1c(S)c(C(C)=O)cc(C(C)=O)c1S] | 2-((1E,3E)-hexa-1,3,5-trien- 1-yl), 3-sulfanyl, 4,6- diacetyl, thiophenol | estimated pKa: 5.5; 6.4 estimated logP (neutral/ionized): 3.1/1.6; 0.2 estimated minimal projection area: 46 Å² estimated z-length: 15 Å predicted rate of energy dissipation: 371 × 10⁶ a.u. of power |
| 3-3 | 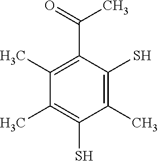 | 2,5,6-trimethyl, 3-sulfanyl, 4-acetyl, thiophenol | estimated pKa: 5.9; 6.9 estimated logP (neutral/ionized): 3.3/1.8; 0.4 estimated minimal projection area: 38 Å² estimated z-length: 10 Å predicted rate of energy dissipation: 758 × 10⁶ a.u. of power |
| 3-4 | 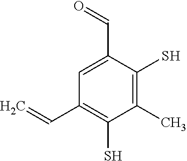 | 2-methyl, 3-sulfanyl, 4- formyl, 6-ethenyl, thiophenol | estimated pKa: 5.7; 6.7 estimated logP (neutral/ionized): 3.1/1.7; 1.7; 0.3 estimated minimal projection area: 34 Å² estimated z-length: 11 Å predicted rate of energy dissipation: 1004 × 10⁶ a.u. of power |
| 3-5 | 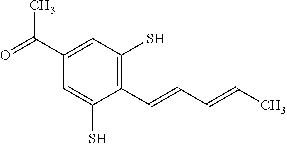 [SMILES: C/C=C/C=C/c1c(S)cc(C(C)=O)cc1S] | 2-((1E,3E)-penta-1,3-dien- 1-yl), 3-sulfanyl, 5-acetyl, thiophenol | estimated pKa: 5.8; 6.8 estimated logP (neutral/ionized): 3.4/1.9; 0.5 estimated minimal projection area: 36 Å² estimated z-length: 15 Å predicted rate of energy dissipation: 535 × 10⁶ a.u. of power |
| 3-6 | 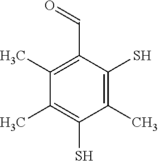 | 2,5,6-trimethyl, 3-sulfanyl, 4-formyl, thiophenol | estimated pKa: 5.6; 6.9 estimated logP (neutral/ionized): 3.4/2.0; 2.0; 0.6 estimated minimal projection area: 35 Å² estimated z-length: 9 Å predicted rate of energy dissipation: 810 × 10⁶ a.u. of power |
| 4-1 | 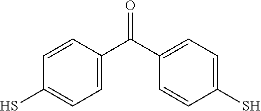 [SMILES: O=C(c1ccc(S)cc1)c1ccc(S)cc1] | 4-[(4- sulfanylphenyl)carbonyl] benzenethiol PubChem. CID 15147116 | estimated pKa: 5.5; 6.5 estimated logP (neutral/ionized): 3.6/0.8 estimated minimal projection area: 27 Å² estimated z-length: 15 Å predicted rate of energy dissipation: 711 × 10⁶ a.u. of power |
| 5-1 | 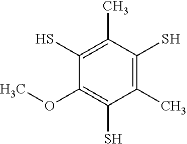 | 1,3,5-trisulfanyl, 2,4- dimethyl, 6-methoxy, benzene | estimated pKa: 5.6; 6.5; 7.5 estimated logP (neutral/ionized): 3.1/1.7; 1.7; 0.3; 0.3; −1.2 estimated minimal projection area: 37 Å² estimated z-length: 11 Å predicted rate of energy dissipation: 1048 × 10⁶ a.u. of power |
| 5-2 | 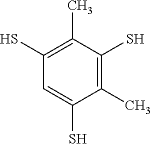 | 1,3,5-trisulfanyl, 2,4- dimethyl, benzene | estimated pKa: 5.8; 6.9; 7.9 estimated logP (neutral/ionized): 3.3/1.9; 1.9; 0.4; 0.4; −1.0 estimated minimal projection area: 32 Å² estimated z-length: 10 Å predicted rate of energy dissipation: 1142 × 10⁶ a.u. of power |
| 5-3 | 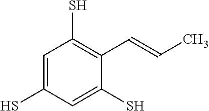 | 1,3,5-trisulfanyl, 4- (propen-1-yl), benzene | estimated pKa: 5.7; 6.7; 7.7 estimated logP (neutral/ionized): 3.4/2.0; 2.0; 0.5; 0.5; −0.9 estimated minimal projection area: 33 Å² estimated z-length: 11 Å predicted rate of energy dissipation: 985 × 10⁶ a.u. of power |
| 23-1 | 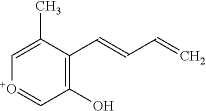 | 3-hydroxy, 4-[(1E)-buta- 1,3-dien-1-yl], 5-methyl, pyrilium | estimated pKa: 4.5 estimated logP (neutral/ionized): 3.1/1.8 estimated minimal projection area: 28 Å² estimated z-length: 11 Å predicted rate of energy dissipation: 946 × 10⁶ a.u. of power |
| 23-2 | 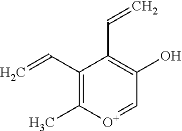 | 3-hydroxy, 4,5-diethenyl, 6-methyl, pyrilium | estimated pKa: 5.2 estimated logP (neutral/ionized): 3.2/1.8 estimated minimal projection area: 33 Å² estimated z-length: 9 Å predicted rate of energy dissipation: 875 × 10⁶ a.u. of power |
| 23-3 | 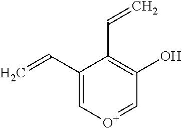 | 3-hydroxy, 4,5-diethenyl, pyrilium | estimated pKa: 4.4 estimated logP (neutral/ionized): 3.0/1.7 estimated minimal projection area: 27 Å² estimated z-length: 10 Å predicted rate of energy dissipation: 997 × 10⁶ a.u. of power |
| 23-4 | 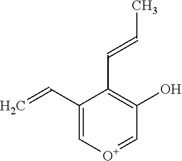 | 3-hydroxy, 4-(propen-1-yl), 5-ethenyl, pyrilium | estimated pKa: 4.4 estimated logP (neutral/ionized): 3.4/2.1 estimated minimal projection area: 31 Å² estimated z-length: 10 Å predicted rate of energy dissipation: 669 × 10⁶ a.u. of power |
| 27-1 | 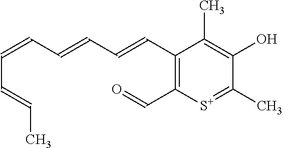 [SMILES: C/C=C/C=C\C=C\C=C\c1c(C=O)[s+]c(C)c(O)c1C] | 2,4-dimethyl, 3-hydroxy, 5- [(1E,3E,5E,7E)-nona- 1,3,5,7-tetraen-1-yl], 6- formyl, thiopyran | estimated pKa: 5.1 estimated logP (neutral/ionized): 3.3/2.0 estimated minimal projection area: 36 Å² estimated z-length: 19 Å predicted rate of energy dissipation: 321 × 10⁶ a.u. of power |
| 27-2 | 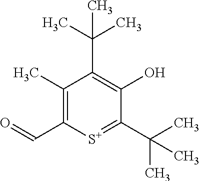 [SMILES: Cc1c(C=O)[s+]c(C(C)(C)C)c(O)c1C(C)(C)C] | 2,4-tert-butyl, 3-hydroxy, 5-methyl, 6-formyl, thiopyran | estimated pKa: 5.9 estimated logP (neutral/ionized): 3.2/1.9 estimated minimal projection area: 47 Å² estimated z-length: 11 Å predicted rate of energy dissipation: 365 × 10⁶ a.u. of power |
| 27-3 | 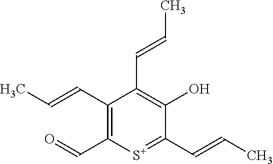 [SMILES: C/C=C/c1[s+]c(C=O)c(/C=C/C)c(/C=C/C)c1O] | 2,4,5-tri-(propen-1-yl), 3- hydroxy, 6-formyl, 27- 4thiopyran | estimated pKa: 4.6 estimated logP (neutral/ionized): 3.3/1.9 estimated minimal projection area: 41 Å² estimated z-length: 14 Å predicted rate of energy dissipation: 383 × 10⁶ a.u. of power |
| 27-4 | 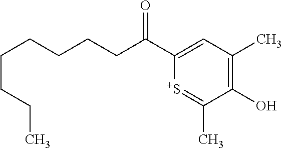 [SMILES: CCCCCCCCC(=O)c1cc(C)c(O)c(C)[s+]1] | 2,4-dimethyl, 3-hydroxy, 6- (nonan-1-one), thiopyran | estimated pKa: 5.6 estimated logP (neutral/ionized): 3.1/1.7 estimated minimal projection area: 37 Å² estimated z-length: 19 Å predicted rate of energy dissipation: 281 × 10⁶ a.u. of power |
| 44-1 | 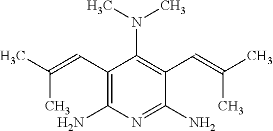 [SMILES: CC(C)=Cc1c(N)nc(N)c(C=C(C)C)c1N(C)C] | 4-N,4-N-dimethyl, 2,4,6- triamine, 3,5-di-(2- methylpropen-1-yl), pyridine | estimated pKa: 10.5 estimated logP (neutral/ionized): 3.1/1.1 estimated minimal projection area: 47 Å² estimated z-length: 13 Å predicted rate of energy dissipation: 49 × 10⁶ a.u. of power |
| 44-2 | 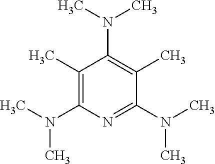 | 2-N,2-N,4-N,4-N,6-N,6-N- hexamethyl, 2,4,6-triamine, 3,5-dimethyl, pyridine | estimated pKa: 11.0 estimated logP (neutral/ionized): 3.3/1.3 estimated minimal projection area: 49 Å² estimated z-length: 11 Å predicted rate of energy dissipation: 91 × 10⁶ a.u. of power |
| 51-1 | 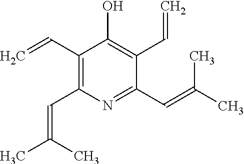 [SMILES: C=Cc1c(C=C(C)C)nc(C=C(C)C)c(C=C)c1O] | 2,6-di-(2-methylpropen-1- yl), 3,5-diethenyl, 4- hydroxy, pyridine | estimated pKa: 10.8 estimated logP (neutral/ionized): 5.1/3.2 estimated minimal projection area: 50 Å² estimated z-length: 11 Å predicted rate of energy dissipation: 233 × 10⁶ a.u. of power |
| 53-1 | 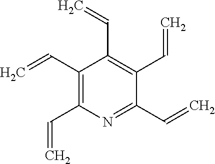 [SMILES: C=Cc1nc(C=C)c(C=C)c(C=C)c1C=C] | 2,3,4,5,6-pentaethenyl, pyridine | estimated pKa: 4.9 estimated logP (neutral/ionized): 4.9/3.0 estimated minimal projection area: 34 Å² estimated z-length: 11 Å predicted rate of energy dissipation: 442 × 10⁶ a.u. of power |

TABLE 4

| Compound | | | |
|---|---|---|---|
| Number | Chemical Structure | Chemical Name | Comments |
| 6-1 | 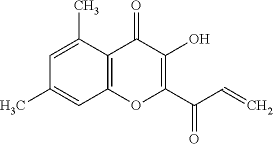 [SMILES: C=CC(=O)c1oc2cc(C)cc(C)c2c(=O)c1O] | 2-(but-3-en-2-one), 3- hydroxy, 5,7-dimethyl, chromone | estimated pKa: 4.1 estimated logP (neutral/ionized): 3.2/−0.4 estimated minimal projection area: 31 Å² estimated z-length: 13 Å predicted rate of energy dissipation: 406 × 10⁶ a.u. of power |
| 6-2 | 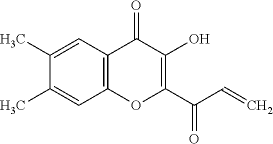 [SMILES: C=CC(=O)c1oc2cc(C)c(C)cc2c(=O)c1O] | 2-(prop-2-en-1-one), 3- hydroxy, 6,7-dimethyl, chromone | estimated pKa: 4.0 estimated logP (neutral/ionized): 3.2/ −0.4 estimated minimal projection area: 31 Å² estimated z-length: 13 Å predicted rate of energy dissipation: 385 × 10⁶ a.u. of power |
| 6-3 | 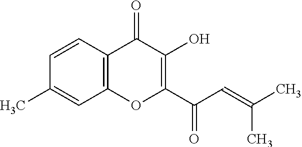 [SMILES: CC(C)=CC(=O)c1oc2cc(C)ccc2c(=O)c1O] | 2-(2-methyl-prop-2-en-1- one), 3-hydroxy, 7-methyl, chromone | estimated pKa: 4.1 estimated logP (neutral/ionized): 3.3/−0.3 estimated minimal projection area: 37 Å² estimated z-length: 14 Å predicted rate of energy dissipation: 245 × 10⁶ a.u. of power |
| 6-4 | 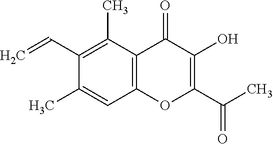 [SMILES: C=Cc1c(C)cc2oc(C(C)=O)c(O)c(=O)c2c1C] | 2-acetyl, 3-hydroxy, 5,7- dimethyl, 6-ethenyl, chromone | estimated pKa: 4.0 estimated logP (neutral/ionized): 3.1/ −0.4 estimated minimal projection area: 38 Å² estimated z-length: 14 Å predicted rate of energy dissipation: 241 × 10⁶ a.u. of power |
| 6-5 | 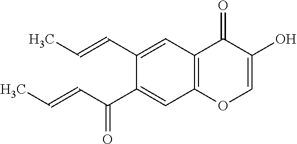 [SMILES: C/C=C/C(=O)c1cc2occ(O)c(=O)c2cc1/C=C/C] | 3-hydroxy, 6-(propen-1- yl),7-(but-2-en-1-one), chromone | estimated pKa: 5.0 estimated logP (neutral/ionized): 3.3/ −0.3 estimated minimal projection area: 42 Å² estimated z-length: 12 Å predicted rate of energy dissipation: 146 × 10⁶ a.u. of power |
| 6-6 | 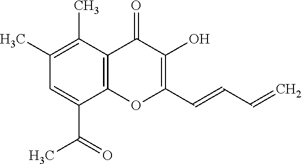 [SMILES: C=C/C=C/c1oc2c(C(C)=O)cc(C)c(C)c2c(=O)c1O] | 2-((1E)-buta-1,3-dien-1-yl), 3-hydroxy, 5,6-dimethyl, 8- acetyl, chromone | estimated pKa: 5.5 estimated logP (neutral/ionized): 3.1/−0.4 estimated minimal projection area: 40 Å² estimated z-length: 14 Å predicted rate of energy dissipation: 85 × 10⁶ a.u. of power |
| 6-7 | 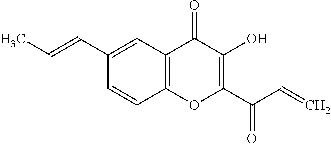 [SMILES: C=CC(=O)c1oc2ccc(/C=C/C)cc2c(=O)c1O] | 2-(prop-2-en-1-one), 3- hydroxy, 6-(propen-1-yl), chromone | estimated pKa: 3.9 estimated logP (neutral/ionized): 3.3/ −0.3 estimated minimal projection area: 30 Å² estimated z-length: 15 Å predicted rate of energy dissipation: 341 × 10⁶ a.u. of power |
| 6-8 | 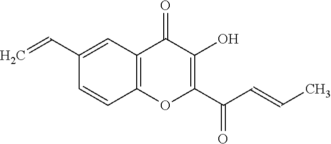 [SMILES: C=Cc1ccc2oc(C(=O)/C=C/C)c(O)c(=O)c2c1] | 2-(but-2-en-1-one), 3- hydroxy, 6-ethenyl, chromone | estimated pKa: 3.9 estimated logP (neutral/ionized): 3.3/−0.3 estimated minimal projection area: 35 Å² estimated z-length: 16 Å predicted rate of energy dissipation: 251 × 10⁶ a.u. of power |
| 6-9 | 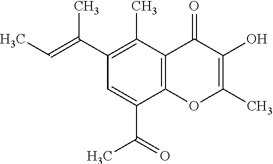 [SMILES: C/C=C(\C)c1cc(C(C)=O)c2oc(C)c(O)c(=O)c2c1C] | 2,5-dimethyl, 6-((2E)-but-2- en-2-yl), 8-acetyl, chromone | estimated pKa: 5.1 estimated logP (neutral/ionized): 3.1/ −0.4 estimated minimal projection area: 45 Å² estimated z-length: 13 Å predicted rate of energy dissipation: 94 × 10⁶ a.u. of power |
| 7-1 | 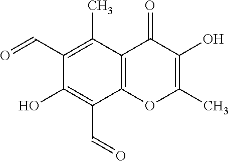 [SMILES: Cc1oc2c(C=O)c(O)c(C=O)c(C)c2c(=O)c1O] | 2,3,5-trimethyl, 6,8- diformyl, 7-hydroxy, chromone | estimated pKa: 5.2 estimated logP (neutral/ionized): 3.2/0.8 estimated minimal projection area: 36 Å² estimated z-length: 11 Å predicted rate of energy dissipation: 351 × 10⁶ a.u. of power |
| 7-2 | 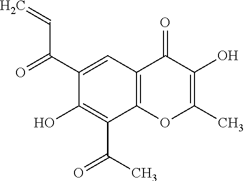 [SMILES: C=CC(=O)c1cc2c(=O)c(O)c(C)oc2c(C(C)=O)c1O] | 2,3-dimethyl, 6-(prop-2-en- 1-one), 7-hydroxy, 8-acetyl, chromone | estimated pKa: 5.1 estimated logP (neutral/ionized): 3.1/0.8 estimated minimal projection area: 36 Å² estimated z-length: 13 Å predicted rate of energy dissipation: 278 × 10⁶ a.u. of power |
| 7-3 | 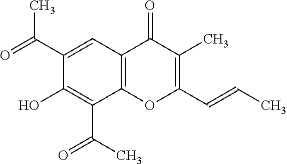 [SMILES: C/C=C/c1oc2c(C(C)=O)c(O)c(C(C)=O)cc2c(=O)c1C] | 2-(propen-1-yl), 3-methyl, 6,8-diacetyl, 7-hydroxy, chromone | estimated pKa: 5.0 estimated logP (neutral/ionized): 3.1/0.8 estimated minimal projection area: 39 Å² estimated z-length: 14 Å predicted rate of energy dissipation: 233 × 10⁶ a.u. of power |
| 7-4 | 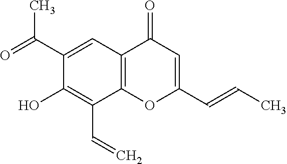 [SMILES: C=Cc1c(O)c(C(C)=O)cc2c(=O)cc(/C=C/C)oc12] | 2-(propen-1-yl), 6-acetyl, 7-hydroxy, 8-ethenyl, chromone | estimated pKa: 6.2 estimated logP (neutral/ionized): 3..3/1.1 estimated minimal projection area: 34 Å² estimated z-length: 15 Å predicted rate of energy dissipation: 190 × 10⁶ a.u. of power |
| 7-5 | 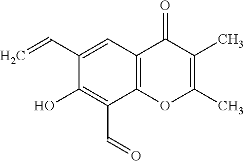 [SMILES: C=Cc1cc2c(=O)c(C)c(C)oc2c(C=O)c1O] | 2,3-dimethyl, 6-ethenyl, 7-hydroxy, 8-formyl, chromone | estimated pKa: 6.2 estimated logP (neutral/ionized): 3.1/0.8 estimated minimal projection area: 35 Å² estimated z-length: 11 Å predicted rate of energy dissipation: 184 × 10⁶ a.u. of power |
| 7-6 | 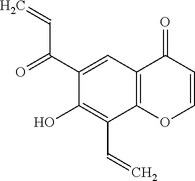 [SMILES: C=CC(=O)c1cc2c(=O)ccoc2c(C=C)c1O] | 6-(prop-2-en-1-one), 7-hydroxy, 8-ethenyl, chromone | estimated pKa: 6.3 estimated logP (neutral/ionized): 3.1/0.8 estimated minimal projection area: 34 Å² estimated z-length: 12 Å predicted rate of energy dissipation: 179 × 10⁶ a.u. of power |
| 7-7 | 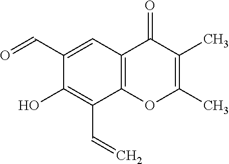 [SMILES: C=Cc1c(O)c(C=O)cc2c(=O)c(C)c(C)oc12] | 2,3-dimethyl, 6-formyl, 7-hydroxy, 8-ethenyl, chromone | estimated pKa: 6.3 estimated logP (neutral/ionized): 3.1/0.8 estimated minimal projection area: 36 Å² estimated z-length: 11 Å predicted rate of energy dissipation: 176 × 10⁶ a.u. of power |
| 7-8 | 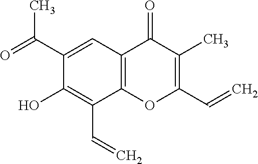 [SMILES: C=Cc1oc2c(C=C)c(O)c(C(C)=O)cc2c(=O)c1C] | 2,8-diethenyl, 3-methyl, 6-acetyl, 7-hydroxy, chromone | estimated pKa: 6.2 estimated logP (neutral/ionized): 3.3/1.1 estimated minimal projection area: 37 Å² estimated z-length: 13 Å predicted rate of energy dissipation: 176 × 10⁶ a.u. of power |
| 7-9 | 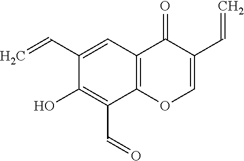 [SMILES: C=Cc1cc2c(=O)c(C=C)coc2c(C=O)c1O] | 3,6-diethenyl, 7-hydroxy, 8-formyl, chromone | estimated pKa: 6.2 estimated logP (neutral/ionized): 3.0/0.8 estimated minimal projection area: 32 Å² estimated z-length: 13 Å predicted rate of energy dissipation: 174 × 10⁶ a.u. of power |
| 7-10 | 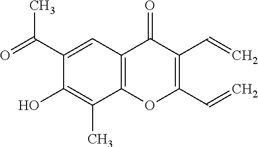 [SMILES: C=Cc1oc2c(C)c(O)c(C(C)=O)cc2c(=O)c1C=C] | 2,3-diethenyl, 6-acetyl, 7- hydroxy, 8- methyl, chromone | estimated pKa: 6.5 estimated logP (neutral/ionized): 3.2/1.0 estimated minimal projection area: 36 Å² estimated z-length: 13 Å predicted rate of energy dissipation: 171 × 10⁶ a.u. of power |
| 7-11 | 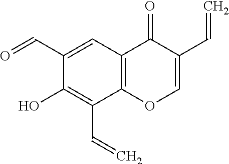 [SMILES: C=Cc1coc2c(C=C)c(O)c(C=O)cc2c1=O] | 3,8-diethenyl, 6-formyl, 7- hydroxy, chromone | estimated pKa: 6.2 estimated logP (neutral/ionized): 3.0/0.8 estimated minimal projection area: 32 Å² estimated z-length: 13 Å predicted rate of energy dissipation: 164 × 10⁶ a.u. of power |
| 7-12 | 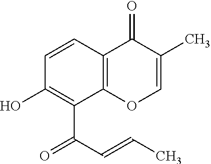 [SMILES: C/C=C/C(=O)c1c(O)ccc2c(=O)c(C)coc12] | 3-methyl, 7-hydroxy, 8- (but-2-en-1-one), chromone | estimated pKa: 6.4 estimated logP (neutral/ionized): 3.1/0.9 estimated minimal projection area: 36 Å² estimated z-length: 12 Å predicted rate of energy dissipation: 162 × 10⁶ a.u. of power |
| 7-13 | 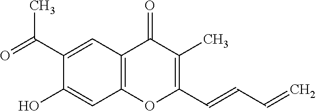 [SMILES: C=C/C=C/c1oc2cc(O)c(C(C)=O)cc2c(=O)c1C] | 2-((1E)-buta-1,3-dien-1-yl), 3-methyl, 6-acetyl, 7- hydroxy, chromone | estimated pKa: 6.1 estimated logP (neutral/ionized): 3.1/0.9 estimated minimal projection area: 36 Å² estimated z-length: 16 Å predicted rate of energy dissipation: 135 × 10⁶ a.u. of power |
| 7-14 | 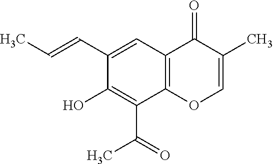 [SMILES: C/C=C/c1cc2c(=O)c(C)coc2c(C(C)=O)c1O] | 3-methyl, 6-(propen-1-yl), 7-hydroxy, 8-acetyl, chromone | estimated pKa: 6.2 estimated logP (neutral/ionized): 3.1/0.9 estimated minimal projection area: 40 Å² estimated z-length: 13 Å predicted rate of energy dissipation: 139 × 10⁶ a.u. of power |
| 7-15 | 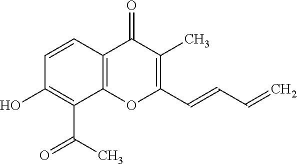 [SMILES: C=C/C=C/c1oc2c(C(C)=O)c(O)ccc2c(=O)c1C] | 2-((1E)-buta-1,3-dien-1-yl), 3-methyl, 7-hydroxy, 8- acetyl, chromone | estimated pKa: 6.3 estimated logP (neutral/ionized): 3.1/0.9 estimated minimal projection area: 42 Å² estimated z-length: 11 Å predicted rate of energy dissipation: 137 × 10⁶ a.u. of power |
| 7-16 | 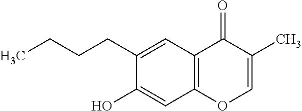 [SMILES: CCCCc1cc2c(=O)c(C)coc2cc1O] | 3-methyl, 6-(butan-1-one), 7-hydroxy, chromone | estimated pKa: 6.1 estimated logP (neutral/ionized): 3.1/0.9 estimated minimal projection area: 37 Å² estimated z-length: 14 Å predicted rate of energy dissipation: 163 × 10⁶ a.u. of power |
| 7-17 | 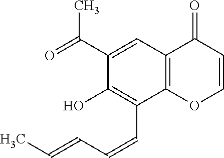 [SMILES: C/C=C/C=C\c1c(O)c(C(C)=O)cc2c(=O)ccoc12] | 6-acetyl, 7-hydroxy, 8- ((1E,3E)-penta-1,3-dien-1- yl), chromone | estimated pKa: 6.2 estimated logP (neutral/ionized): 3.2/1.0 estimated minimal projection area: 39 Å² estimated z-length: 14 Å predicted rate of energy dissipation: 166 × 10⁶ a.u. of power |
| 8-1 | 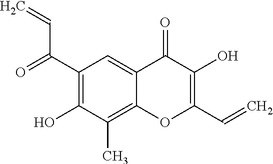 [SMILES: C=CC(=O)c1cc2c(=O)c(O)c(C=C)oc2c(C)c1O] | 2-ethenyl, 3,7-dihydroxy, 6-(prop-2-en-1-one), 8- methyl, chromone | estimated pKa: 5.2; 6.7 estimated logP (neutral/ionized): 3.2/0.9; −0.4; −2.6 estimated minimal projection area: 33 Å² estimated z-length: 14 Å predicted rate of energy dissipation: 208 × 10⁶ a.u. of power |
| 8-2 | 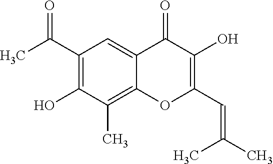 [SMILES: CC(=O)c1cc2c(=O)c(O)c(C=C(C)C)oc2c(C)c1O] | 2-(2-methylprop-1-en-1-yl), 3,7-dihydroxy, 6-acetyl, 8- methyl, chromone | estimated pKa: 5.3; 6.7 estimated logP (neutral/ionized): 3.1/0.8; −0.5; −2.7 estimated minimal projection area: 36 Å² estimated z-length: 15 Å predicted rate of energy dissipation: 122 × 10⁶ a.u. of power |
| 8-3 | 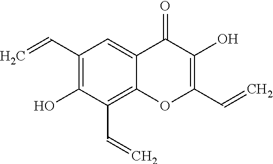 [SMILES: C=Cc1cc2c(=O)c(O)c(C=C)oc2c(C=C)c1O] | 2,6,8-triethenyl, 3,7- dihydroxy, chromone | estimated pKa: 5.5; 7.5 estimated logP (neutral/ionized): 3.2/1.1; −0.4; −2.5 estimated minimal projection area: 35 Å² estimated z-length: 13 Å predicted rate of energy dissipation: 137 × 10⁶ a.u. of power |
| 8-4 | 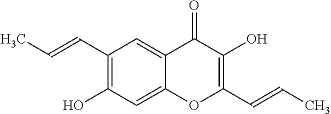 [SMILES: C/C=C/c1cc2c(=O)c(O)c(/C=C/C)oc2cc1O] | 2,6-di-(propen-1-yl), 3,7- dihydroxy, chromone | estimated pKa: 5.5; 7.4 estimated logP (neutral/ionized): 3.2/1.1; −0.3; −2.4 estimated minimal projection area: 32 Å² estimated z-length: 15 Å predicted rate of energy dissipation: 138 × 10⁶ a.u. of power |
| 9-1 | 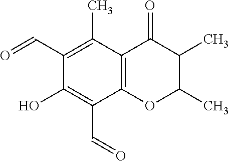 [SMILES: Cc1c(C=O)c(O)c(C=O)c2c1C(=O)C(C)C(C)O2] | 2,3,5-trimethyl, 6,8- diformyl, 7-hydroxy, dihydrochromone | estimated pKa: 5.2 estimated logP (neutral/ionized): 3.2/0.8 estimated minimal projection area: 40 Å² estimated z-length: 11 Å predicted rate of energy dissipation: 290 × 10⁶ a.u. of power |
| 9-2 | 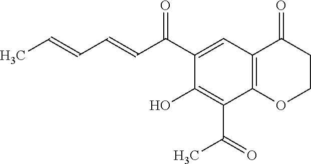 [SMILES: C/C=C/C=C/C(=O)c1cc2c(c(C(C)=O)c1O)OCCC2=O] | 6-((2E,4E)-hexa-2,4-dien-1- one), 7-hydroxy, 8-acetyl, dihydrochromone | estimated pKa: 5.0 estimated logP (neutral/ionized): 3.1/0.7 estimated minimal projection area: 39 Å² estimated z-length: 16 Å predicted rate of energy dissipation: 177 × 10⁶ a.u. of power |
| 9-3 | 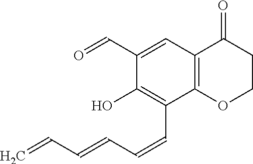 [SMILES: C=C/C=C/C=C\c1c(O)c(C=O)cc2c1OCCC2=O] | 6-formyl, 7-hydroxy, 8- ((1E,3E,5E)-hexa-1,3,5- trien-1-yl), dihydrochromone | estimated pKa: 6.2 estimated logP (neutral/ionized): 3.2/0.9 estimated minimal projection area: 36 Å² estimated z-length: 15 Å predicted rate of energy dissipation: 164 × 10⁶ a.u. of power |
| 9-4 | 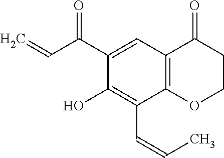 [SMILES: C=CC(=O)c1cc2c(c(/C=C\C)c1O)OCCC2=O] | 6-(prop-2-en-1-one), 7- hydroxy, 8-(propen-1-yl), dihydrochromone | estimated pKa: 6.2 estimated logP (neutral/ionized): 3.1/0.9 estimated minimal projection area: 41 Å² estimated z-length: 13 Å predicted rate of energy dissipation: 127 × 10⁶ a.u. of power |
| 9-5 | 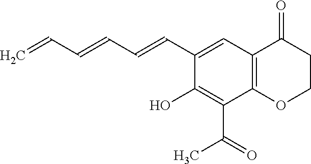 [SMILES: C=C/C=C/C=C/c1cc2c(c(C(C)=O)c1O)OCCC2=O] | 6-((1E,3E,5E)-hexa-1,3,5- trien-1-yl), 7-hydroxy, 8- acetyl, dihydrochromone | estimated pKa: 6.1 estimated logP (neutral/ionized): 3.0/0.8 estimated minimal projection area: 40 Å² estimated z-length: 16 Å predicted rate of energy dissipation: 93 × 10⁶ a.u. of power |
| 9-6 | 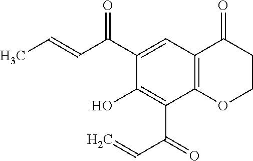 [SMILES: C=CC(=O)c1c(O)c(C(=O)/C=C/C)cc2c1OCCC2=O] | 6-(but-2-en-1-one), 7- hydroxy, 8-(prop-2-en-1- one), dihydrochromone | estimated pKa: 5.1 estimated logP (neutral/ionized): 3.3/1.0 estimated minimal projection area: 42 Å² estimated z-length: 14 Å predicted rate of energy dissipation: 182 × 10⁶ a.u. of power |
| 9-7 | 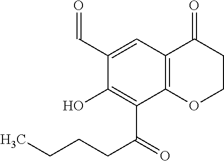 [SMILES: CCCCC(=O)c1c(O)c(C=O)cc2c1OCCC2=O] | 6-formyl, 7-hydroxy, 8- (pentan-1-one), dihydrochromone | estimated pKa: 5.0 estimated logP (neutral/ionized): 3.2/0.8 estimated minimal projection area: 39 Å² estimated z-length: 15 Å predicted rate of energy dissipation: 232 × 10⁶ a.u. of power |
| 10-1 | 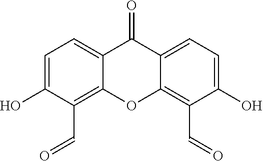 [SMILES: O=Cc1c(O)ccc2c(=O)c3ccc(O)c(C=O)c3oc12] | 3,6-dihydroxy-9- oxoxanthene-4,5- dicarbaldehyde | estimated pKa: 5.6; 6.2 estimated logP (neutral/ionized): 3.1/0.9; −1.4 estimated minimal projection area: 33 Å² estimated z-length: 12 Å predicted rate of energy dissipation: 490 × 10⁶ a.u. of power |
| 10-2 | 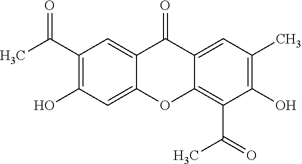 [SMILES: CC(=O)c1cc2c(=O)c3cc(C)c(O)c(C(C)=O)c3oc2cc1O] | 4,7-diacetyl-3,6-dihydroxy- 2-methylxanthen-9-one | estimated pKa: 5.6; 6.3 estimated logP (neutral/ionized): 3.3/1.1; 1.1; −1.2 estimated minimal projection area: 36 Å² estimated z-length: 15 Å predicted rate of energy dissipation: 425 × 10⁶ a.u. of power |
| 10-3 | 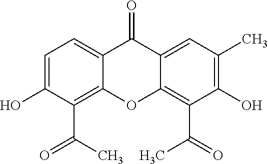 [SMILES: CC(=O)c1c(O)ccc2c(=O)c3cc(C)c(O)c(C(C)=O)c3oc12] | 4,7-diacetyl-3,6-dihydroxy- 2-methylxanthen-9-one | estimated pKa: 5.7; 6.4 estimated logP (neutral/ionized): 3.3/1.1; 1.1; −1.2 estimated minimal projection area: 38 Å² estimated z-length: 13 Å predicted rate of energy dissipation: 404 × 10⁶ a.u. of power |
| 11-1 | 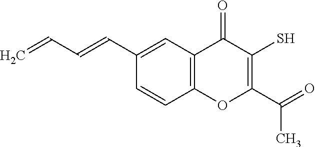 [SMILES: C=C/C=C/c1ccc2oc(C(C)=O)c(S)c(=O)c2c1] | 2-acetyl, 3-sulfanyl, 6-((1E)- buta-1,3-dien-1-yl), chromone | estimated pKa: 4.9 estimated logP (neutral/ionized): 3.2/2.0 estimated minimal projection area: 35 Å² estimated z-length: 16 Å predicted rate of energy dissipation: 379 × 10⁶ a.u. of power |
| 11-2 | 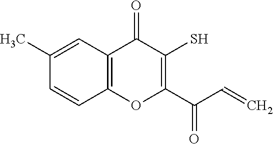 [SMILES: C=CC(=O)c1oc2ccc(C)cc2c(=O)c1S] | 2-(prop-2-en-1-one), 3- sulfanyl, 6-methyl, chromone | estimated pKa: 4.9 estimated logP (neutral/ionized): 3.2/2.0 estimated minimal projection area: 30 Å² estimated z-length: 13 Å predicted rate of energy dissipation: 607 × 10⁶ a.u. of power |
| 11-3 | 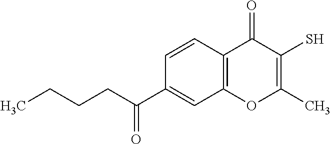 [SMILES: CCCCC(=O)c1ccc2c(=O)c(S)c(C)oc2c1] | 2-methyl, 3-sulfanyl, 7- (pentan-1-one), chromone | estimated pKa: 5.5 estimated logP (neutral/ionized): 3.3/2.1 estimated minimal projection area: 38 Å² estimated z-length: 16 Å predicted rate of energy dissipation: 250 × 10⁶ a.u. of power |
| 12-1 | 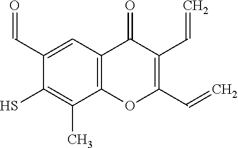 [SMILES: C=Cc1oc2c(C)c(S)c(C=O)cc2c(=O)c1C=C] | 2,3-diethenyl, 6-formyl, 7- sulfanyl, 8-methyl, chromone | estimated pKa: 5.7 estimated logP (neutral/ionized): 3.1/1.7 estimated minimal projection area: 35 Å² estimated z-length: 12 Å predicted rate of energy dissipation: 323 × 10⁶ a.u. of power |
| 12-2 | 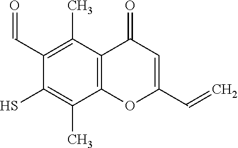 [SMILES: C=Cc1cc(=O)c2c(C)c(C=O)c(S)c(C)c2o1] | 2-ethenyl, 5,8-dimethyl, 6- formyl, 7-sulfanyl, chromone | estimated pKa: 5.7 estimated logP (neutral/ionized): 3.1/1.7 estimated minimal projection area: 36 Å² estimated z-length: 12 Å predicted rate of energy dissipation: 296 × 10⁶ a.u. of power |
| 12-3 | 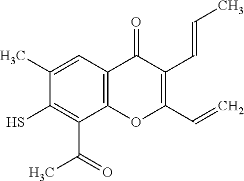 [SMILES: C=Cc1oc2c(C(C)=O)c(S)c(C)cc2c(=O)c1/C=C/C] | 2-(propen-1-yl), 3-ethenyl, 6-methyl, 7-sulfanyl, 8- acetyl, chromone | estimated pKa: 5.8 estimated logP (neutral/ionized): 3.3/1.9 estimated minimal projection area: 40 Å² estimated z-length: 14 Å predicted rate of energy dissipation: 215 × 10⁶ a.u. of power |
| 12-4 | 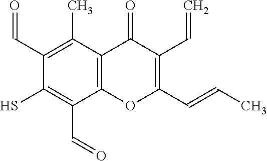 [SMILES: C=Cc1c(/C=C/C)oc2c(C=O)c(S)c(C=O)c(C)c2c1=O] | 2-(propen-1-yl), 3-ethenyl, 5-methyl, 6,8-diformyl, 7- sulfanyl, chromone | estimated pKa: 5.5 estimated logP (neutral/ionized): 3.2/1.8 estimated minimal projection area: 44 Å² estimated z-length: 13 Å predicted rate of energy dissipation: 256 × 10⁶ a.u. of power |
| 13-1 | 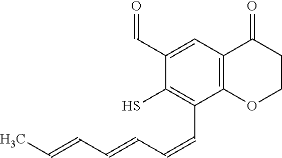 [SMILES: C/C=C/C=C/C=C\c1c(S)c(C=O)cc2c1OCCC2=O] | 6-formyl, 7-sulfanyl, 8- ((1E,3E,5E)-hepta-1,3,5- trien-1-yl), dihydrochromone | estimated pKa: 5.8 estimated logP (neutral/ionized): 3.3/1.9 estimated minimal projection area: 42 Å² estimated z-length: 17 Å predicted rate of energy dissipation: 177 × 10⁶ a.u. of power |
| 13-2 | 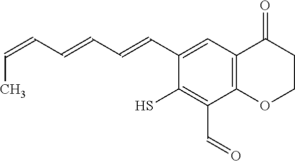 [SMILES: C\C=C/C=C/C=C/c1cc2c(c(C=O)c1S)OCCC2=O] | 6-((1E,3E,5E)-hepta,1,3,5- trien-1-yl), 7-sulfanyl, 8- formyl dihydrochromone | estimated pKa: 5.8 estimated logP (neutral/ionized): 3.3/1.9 estimated minimal projection area: 41 Å² estimated z-length: 17 Å predicted rate of energy dissipation: 182 × 10⁶ a.u. of power |
| 13-3 | 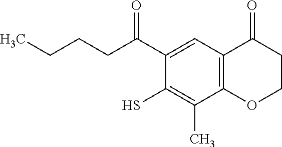 [SMILES: CCCCC(=O)c1cc2c(c(C)c1S)OCCC2=O] | 6-(pentan-1-one), 7- sulfanyl, 8-methyl, dihydrochromone | estimated pKa: 5.8 estimated logP (neutral/ionized): 3.1/1.6 estimated minimal projection area: 39 Å² estimated z-length: 15 Å predicted rate of energy dissipation: 194 × 10⁶ a.u. of power |
| 13-4 | 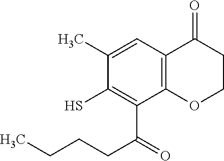 [SMILES: CCCCC(=O)c1c(S)c(C)cc2c1OCCC2=O] | 6-methyl, 7-sulfanyl, 8- (pentan-1-one), dihydrochromone | estimated pKa: 5.8 estimated logP (neutral/ionized): 3.1/1.6 estimated minimal projection area: 40 Å² estimated z-length: 13 Å predicted rate of energy dissipation: 201 × 10⁶ a.u. of power |
| 14-1 | 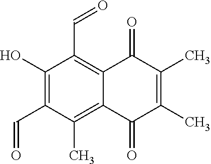 [SMILES: CC1=C(C)C(=O)c2c(C=O)c(O)c(C=O)c(C)c2C1=O] | 2,3,8-trimethyl, 5,7- diformyl, 6-hydroxy, 1,4- naphtoquinone | estimated pKa: 5.1 estimated logP (neutral/ionized): 3.2/0.9 estimated minimal projection area: 37 Å² estimated z-length: 12 Å predicted rate of energy dissipation: 319 × 10⁶ a.u. of power |
| 14-2 | 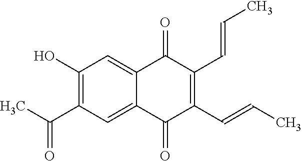 [SMILES: C/C=C/C1=C(/C=C/C)C(=O)c2cc(C(C)=O)c(O)cc2C1=O] | 2,3-di-(propen-1-yl), 6- hydroxy, 7-acetyl, 1,4- naphtoquinone | estimated pKa: 6.0 estimated logP (neutral/ionized): 3.2/1.0 estimated minimal projection area: 40 Å² estimated z-length: 15 Å predicted rate of energy dissipation: 164 × 10⁶ a.u. of power |
| 14-3 | 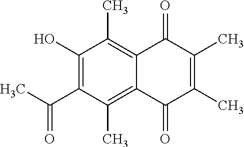 [SMILES: CC(=O)c1c(C)c2c(c(C)c1O)C(=O)C(C)=C(C)C2=O] | 2,3,5,8-tetramethyl, 6- hydroxy, 7-acetyl, 1,4- naphtoquinone | estimated pKa: 6.6 estimated logP (neutral/ionized): 3.2/1.0 estimated minimal projection area: 39 Å² estimated z-length: 12 Å predicted rate of energy dissipation: 145 × 10⁶ a.u. of power |
| 14-4 | 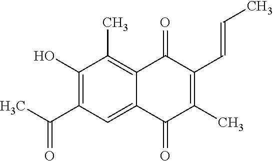 [SMILES: C/C=C/C1=C(C)C(=O)c2cc(C(C)=O)c(O)c(C)c2C1=O] | 2,5-dimethyl, 3-(propen-1- yl), 6-hydroxy, 7-acetyl, 1,4-naphtoquinone | estimated pKa: 6.5 estimated logP (neutral/ionized): 3.2/1.0 estimated minimal projection area: 37 Å² estimated z-length: 14 Å predicted rate of energy dissipation: 153 × 10⁶ a.u. of power |
| 14-5 | 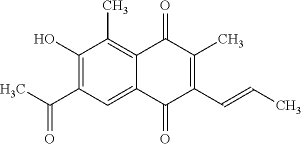 [SMILES: C/C=C/C1=C(C)C(=O)c2c(cc(C(C)=O)c(O)c2C)C1=O] | 2-(propen-1-yl), 3,5- dimethyl, 6-hydroxy, 7- acetyl, 1,4-naphtoquinone | estimated pKa: 6.5 estimated logP (neutral/ionized): 3.2/1.0 estimated minimal projection area: 41 Å² estimated z-length: 14 Å predicted rate of energy dissipation: 128 × 10⁶ a.u. of power |
| 14-6 | 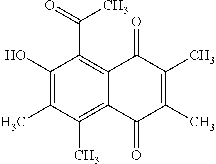 [SMILES: CC(=O)c1c(O)c(C)c(C)c2c1C(=O)C(C)=C(C)C2=O] | 2,3,7,8-tetramethyl, 5- acetyl, 6-hydroxy, 1,4- naphtoquinone | estimated pKa: 6.6 estimated logP (neutral/ionized): 3.2/1.0 estimated minimal projection area: 44 Å² estimated z-length: 11 Å predicted rate of energy dissipation: 121 × 10⁶ a.u. of power |
| 14-7 | 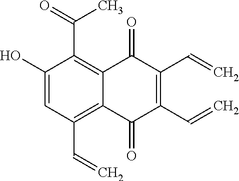 [SMILES: C=CC1=C(C=C)C(=O)c2c(C(C)=O)c(O)cc(C=C)c2C1=O] | 2,3,8-triethenyl, 5-acetyl, 6- hydroxy, 1,4- naphtoquinone | estimated pKa: 6.0 estimated logP (neutral/ionized): 3.2/1.0 estimated minimal projection area: 49 Å² estimated z-length: 12 Å predicted rate of energy dissipation: 131 × 10⁶ a.u. of power |
| 14-8 | 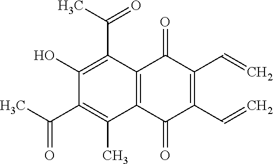 [SMILES: C=CC1=C(C=C)C(=O)c2c(c(C)c(C(C)=O)c(O)c2C(C)=O)C1=O] | 2,3-diethenyl, 5,7-diacetyl, 6-hydroxy, 8-methtyl, 1,4- naphtoquinone | estimated pKa: 5.0 estimated logP (neutral/ionized): 3.2/0.8 estimated minimal projection area: 45 Å² estimated z-length: 13 Å predicted rate of energy dissipation: 218 × 10⁶ a.u. of power |
| 14-9 | 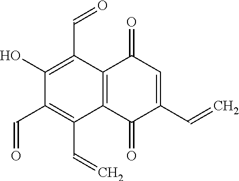 [SMILES: C=CC1=CC(=O)c2c(C=O)c(O)c(C=O)c(C=C)c2C1=O] | 2,8-diethenyl, 5,7-diformyl, 6-hydroxy, 1,4- naphtoquinone | estimated pKa: 5.0 estimated logP (neutral/ionized): 3.2/0.8 estimated minimal projection area: 39 Å² estimated z-length: 12 Å predicted rate of energy dissipation: 294 × 10⁶ a.u. of power |
| 14-10 | 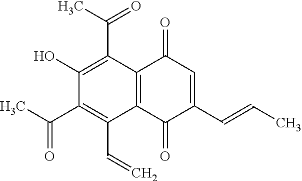 [SMILES: C=Cc1c(C(C)=O)c(O)c(C(C)=O)c2c1C(=O)C(/C=C/C)=CC2=O] | 2-(propen-1-yl), 5,7-acetyl, 6-hydroxy, 8-ethenyl, 1,4- naphtoquinone | estimated pKa: 4.9 estimated logP (neutral/ionized): 3.3/0.9 estimated minimal projection area: 46 Å² estimated z-length: 14 Å predicted rate of energy dissipation: 180 × 10⁶ a.u. of power |
| 15-1 | 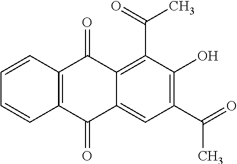 [SMILES: CC(=O)c1cc2c(c(C(C)=O)c1O)C(=O)c1ccccc1C2=O] | 1,3-diacetyl, 2-hydroxy, anthraquinone | estimated pKa: 4.9 estimated logP (neutral/ionized): 3.0/0.7 estimated minimal projection area: 38 Å² estimated z-length: 13 Å predicted rate of energy dissipation: 224 × 10⁶ a.u. of power |
| 15-2 | 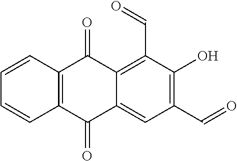 [SMILES: O=Cc1cc2c(c(C=O)c1O)C(=O)c1ccccc1C2=O] | 1,3-formyl, 2-hydroxy, anthraquinone | estimated pKa: 5.0 estimated logP (neutral/ionized): 3.3/1.0 estimated minimal projection area: 35 Å² estimated z-length: 13 Å predicted rate of energy dissipation: 298 × 10⁶ a.u. of power |
| 16-1 | 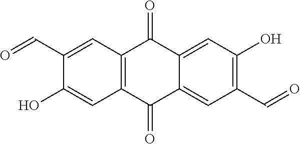 [SMILES: O=Cc1cc2c(cc1O)C(=O)c1cc(C=O)c(O)cc1C2=O] | 2,6-dihydroxy, 3,7-diformyl, anthraquinone | estimated pKa: 5.8; 6.4 estimated logP (neutral/ionized): 3.0/0.8; −1.4 estimated minimal projection area: 35 Å² estimated z-length: 14 Å predicted rate of energy dissipation: 359 × 10⁶ a.u. of power |
| 16-2 | 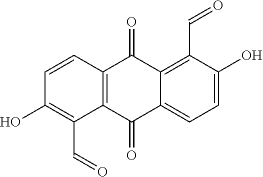 [SMILES: O=Cc1c(O)ccc2c1C(=O)c1ccc(O)c(C=O)c1C2=O] | 2,6-dihydroxy, 1,5-diformyl, anthraquinone | estimated pKa: 5.8; 6.4 estimated logP (neutral/ionized): 3.0/0.8; −1.4 estimated minimal projection area: 30 Å² estimated z-length: 13 Å predicted rate of energy dissipation: 437 × 10⁶ a.u. of power |
| 17-1 | 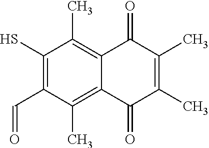 [SMILES: CC1=C(C)C(=O)c2c(C)c(C=O)c(S)c(C)c2C1=O] | 2,3,5,8-tetramethyl, 6- sulfanyl, 7-formyl, 1,4- naphtoquinone | estimated pKa: 5.7 estimated logP (neutral/ionized): 3.1/1.7 estimated minimal projection area: 37 Å² estimated z-length: 12 Å predicted rate of energy dissipation: 324 × 10⁶ a.u. of power |
| 17-2 | 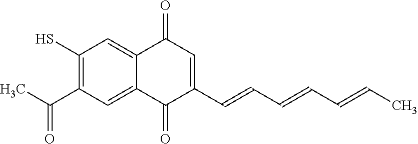 [SMILES: C/C=C/C=C/C=C/C1=CC(=O)c2cc(S)c(C(C)=O)cc2C1=O] | 2-((1E,3E,5E)-hepta-1,3,5- trien-1-yl), 6-sulfanyl, 7- acetyl, 1,4-naphtoquinone | estimated pKa: 5.5 estimated logP (neutral/ionized): 3.1/1.7 estimated minimal projection area: 34 Å² estimated z-length: 19 Å predicted rate of energy dissipation: 237 × 10⁶ a.u. of power |
| 17-3 | 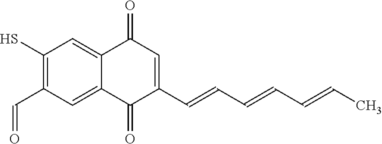 [SMILES: C/C=C/C=C/C=C/C1=CC(=O)c2cc(S)c(C=O)cc2C1=O] | 2-((1E,3E,5E)-hepta-1,3,5- trien-1-yl), 6-sulfanyl, 7- formyl, 1,4-naphtoquinone | estimated pKa: 5.5 estimated logP (neutral/ionized): 3.3/1.9 estimated minimal projection area: 33 Å² estimated z-length: 19 Å predicted rate of energy dissipation: 288 × 10⁶ a.u. of power |
| 17-4 | 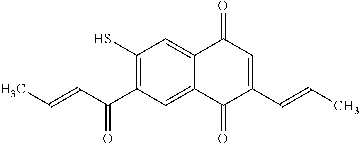 [SMILES: C/C=C/C(=O)c1cc2c(cc1S)C(=O)C=C(/C=C/C)C2=O] | 2-(propen-1-yl), 6-sulfanyl, 7-(but-2-en-1-one), 1,4- naphtoquinone | estimated pKa: 5.7 estimated logP (neutral/ionized): 3.2/1.8 estimated minimal projection area: 40 Å² estimated z-length: 16 Å predicted rate of energy dissipation: 239 × 10⁶ a.u. of power |
| 17-5 | 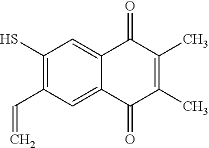 [SMILES: C=Cc1cc2c(cc1S)C(=O)C(C)=C(C)C2=O] | 2,3-dimethyl, 6-sulfanyl, 7- ethenyl, 1,4-naphtoquinone | estimated pKa: 5.9 estimated logP (neutral/ionized): 3.1/1.7 estimated minimal projection area: 34 Å² estimated z-length: 12 Å predicted rate of energy dissipation: 359 × 10⁶ a.u. of power |
| 17-6 | 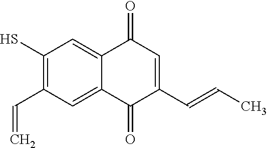 [SMILES: C=Cc1cc2c(cc1S)C(=O)C=C(/C=C/C)C2=O] | 2-(propen-1-yl), 6-sulfanyl, 7-ethenyl, 1,4- naphtoquinone | estimated pKa: 5.9 estimated logP (neutral/ionized): 3.3/1.8 estimated minimal projection area: 36 Å² estimated z-length: 14 Å predicted rate of energy dissipation: 291 × 10⁶ a.u. of power |
| 17-7 | 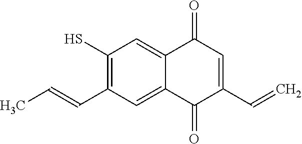 [SMILES: C=CC1=CC(=O)c2cc(S)c(/C=C/C)cc2C1=O] | 2-ethenyl, 6-sulfanyl, 7- (propen-1-yl), 1,4- naphtoquinone | estimated pKa: 5.9 estimated logP (neutral/ionized): 3.3/1.8 estimated minimal projection area: 34 Å² estimated z-length: 14 Å predicted rate of energy dissipation: 338 × 10⁶ a.u. of power |
| 17-8 | 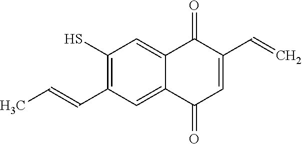 [SMILES: C=CC1=CC(=O)c2cc(/C=C/C)c(S)cc2C1=O] | 3-ethenyl, 6-sulfanyl, 7- (propen-1-yl), 1,4- naphtoquinone | estimated pKa: 5.9 estimated logP (neutral/ionized): 3.3/1.8 estimated minimal projection area: 33 Å² estimated z-length: 15 Å predicted rate of energy dissipation: 339 × 10⁶ a.u. of power |
| 17-9 | 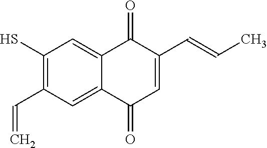 [SMILES: C=Cc1cc2c(cc1S)C(=O)C(/C=C/C)=CC2=O] | 3-(propen-1-yl), 6-sulfanyl, 7-ethenyl, 1,4- naphtoquinone | estimated pKa: 5.9 estimated logP (neutral/ionized): 3.3/1.8 estimated minimal projection area: 32 Å² estimated z-length: 15 Å predicted rate of energy dissipation: 357 × 10⁶ a.u. of power |
| 17-10 | 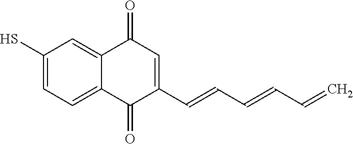 [SMILES: C=C/C=C/C=C/C1=CC(=O)c2cc(S)ccc2C1=O] | 2-((1E,3E,5E)-hexa-1,3,5- trien-1-yl), 6-sulfanyl, 1,4- naphtoquinone | estimated pKa: 5.8 estimated logP (neutral/ionized): 3.2/1.8 estimated minimal projection area: 31 Å² estimated z-length: 17 Å predicted rate of energy dissipation: 339 × 10⁶ a.u. of power |
| 17-11 | 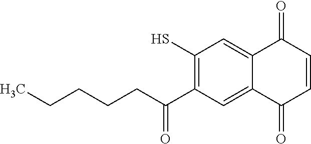 [SMILES: CCCCCC(=O)c1cc2c(cc1S)C(=O)C=CC2=O] | 6-sulfanyl, 7-(hexan-1-one), 1,4-naphtoquinone | estimated pKa: 5.7 estimated logP (neutral/ionized): 3.2/1.8 estimated minimal projection area: 38 Å² estimated z-length: 16 Å predicted rate of energy dissipation: 253 × 10⁶ a.u. of power |
| 18-1 | 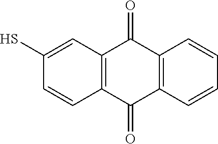 [SMILES: O=C1c2ccccc2C(=O)c2cc(S)ccc21] | 2-sulfanyl, anthracene- 9,10-dione PubChem. CID 22058815; CAS 13354-38-6; | estimated pKa: 5.5 estimated logP (neutral/ionized): 3.2/1.6 estimated minimal projection area: 29 Å² estimated z-length: 13 Å predicted rate of energy dissipation: 407 × 10⁶ a.u. of power |
| 19-1 | 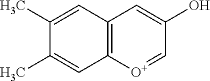 | 3-hydroxy, 6,7-dimethyl, chromenylium | estimated pKa: 5.3 estimated logP (neutral/ionized): 3.13/1.80 estimated minimal projection area: 24 Å² estimated z-length: 11 Å predicted rate of energy dissipation: 1248 × 10⁶ a.u. of power |
| 19-2 | 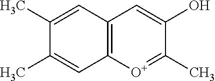 | 3-hydroxy, 2,6,7-trimethyl, chromenylium | estimated pKa: 6.1 estimated logP (neutral/ionized): 3.3/1.9 estimated minimal projection area: 29 Å² estimated z-length: 11 Å predicted rate of energy dissipation: 908 × 10⁶ a.u. of power |
| 19-3 | 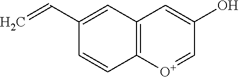 | 3-hydroxy, 6-ethenyl, chromenylium | estimated pKa: 5.0 estimated logP (neutral/ionized): 3.1/1.8 estimated minimal projection area: 26 Å² estimated z-length: 11 Å predicted rate of energy dissipation: 1028 × 10⁶ a.u. of power |
| 19-4 | 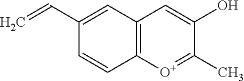 | 2-methyl, 3-hydroxy, 6- ethenyl, chromenylium | estimated pKa: 5.8 estimated logP (neutral/ionized): 3.2/1.9 estimated minimal projection area: 27 Å² estimated z-length: 13 Å predicted rate of energy dissipation: 1011 × 10⁶ a.u. of power |
| 19-5 | 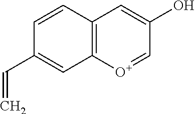 | 3-hydroxy, 7-ethenyl, chromenylium | estimated pKa: 5.1 estimated logP (neutral/ionized): 3.1/1.8 estimated minimal projection area: 23 Å² estimated z-length: 12 Å predicted rate of energy dissipation: 1180 × 10⁶ a.u. of power |
| 19-6 | 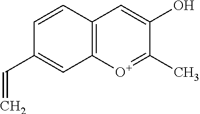 | 2-methyl, 3-hydroxy, 7- ethenyl, chromenylium | estimated pKa: 5.9 estimated logP (neutral/ionized): 3.2/1.9 estimated minimal projection area: 27 Å² estimated z-length: 12 Å predicted rate of energy dissipation: 1024 × 10⁶ a.u. of power |
| 20-1 | 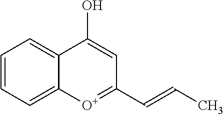 | 2-(propen-1-yl), 4-hydroxy, chromenylium | estimated pKa: 6.5 estimated logP (neutral/ionized): 3.3/2.0 estimated minimal projection area: 29 Å² estimated z-length: 12 Å predicted rate of energy dissipation: 674 × 10⁶ a.u. of power |
| 20-2 | 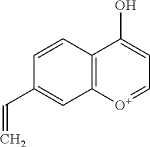 | 4-hydroxy, 7-ethenyl, chromenylium | estimated pKa: 6.9 estimated logP (neutral/ionized): 3.1/1.8 estimated minimal projection area: 25 Å² estimated z-length: 12 Å predicted rate of energy dissipation: 961 × 10⁶ a.u. of power |
| 21-1 | 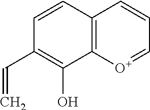 | 7-ethenyl, 8-hydroxy, chromenylium | estimated pKa: 4.8 estimated logP (neutral/ionized): 3.1/0.5 estimated minimal projection area: 24 Å² estimated z-length: 12 Å predicted rate of energy dissipation: 1078 × 10⁶ a.u. of power |
| 21-2 | 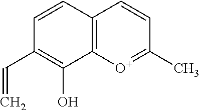 | 2-methyl, 7-ethenyl, 8- hydroxy, chromenylium | estimated pKa: 4.9 estimated logP (neutral/ionized): 3.2/0.6 estimated minimal projection area: 26 Å² estimated z-length: 13 Å predicted rate of energy dissipation: 1070 × 10⁶ a.u. of power |
| 22-1 | 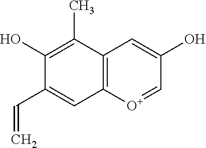 | 3,6-dihydroxy, 5-methyl, 7- ethenyl, chromenylium | estimated pKa: 5.5; 6.6 estimated logP (neutral/ionized): 3.2/2.0; 0.7; −0.6 estimated minimal projection area: 31 Å² estimated z-length: 12 Å predicted rate of energy dissipation: 1712 × 10⁶ a.u. of power |
| 22-2 | 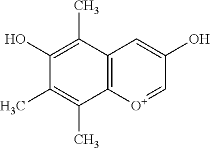 | 3,6-dihydroxy, 5,7,8- trimethyl, chromenylium | estimated pKa: 5.8; 7.5 estimated logP (neutral/ionized): 3.3/2.0; 0.7; −0.6 estimated minimal projection area: 34 Å² estimated z-length: 11 Å predicted rate of energy dissipation: 1097 × 10⁶ a.u. of power |
| 22-3 | 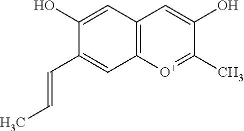 [SMILES: C/C=C/c1cc2[o+]c(C)c(O)cc2cc1O] | 2-methyl, 3,6-dihydroxy, 7- (propen-1-yl), chromenylium | estimated pKa: 5.8; 6.6 estimated logP (neutral/ionized): 3.3/1.9; 0.7; −0.6 estimated minimal projection area: 34 Å² estimated z-length: 13 Å predicted rate of energy dissipation: 1261 × 10⁶ a.u. of power |
| 45-1 | 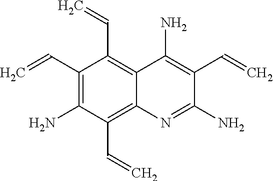 [SMILES: C=Cc1c(N)nc2c(C=C)c(N)c(C=C)c(C=C)c2c1N] | 2,4,7-triamine, 3,5,6,8- tetraethenyl, quinoline | estimated pKa: 10.5 estimated logP (neutral/ionized): 3.1/1.1 estimated minimal projection area: 47 Å² estimated z-length: 13 Å predicted rate of energy dissipation: 49 × 10⁶ a.u. of power |
| 45-2 | 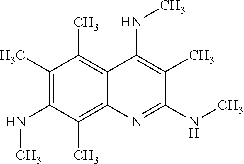 | 2-N,4-N,7-N-trimethyl, 2,4,7-triamine, 3,5,6,8- tetramethyl, quinoline | estimated pKa: 11.0 estimated logP (neutral/ionized): 3.3/1.3 estimated minimal projection area: 49 Å² estimated z-length: 11 Å predicted rate of energy dissipation: 91 × 10⁶ a.u. of power |
| 46-1 | 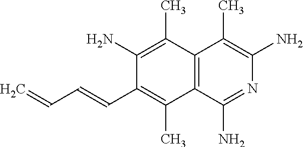 [SMILES: C=C/C=C/c1c(N)c(C)c2c(C)c(N)nc(N)c2c1C] | 2,5,8-triamine, 3,4,7- trimethyl, 6-((1E)-buta-1,3- dien-1-yl), isoquinoline | estimated pKa: 11.0 estimated logP (neutral/ionized): 3.3/1.3 estimated minimal projection area: 39 Å² estimated z-length: 15 Å predicted rate of energy dissipation: 100 × 10⁶ a.u. of power |
| 46-2 | 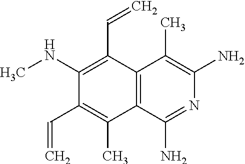 [SMILES: C=Cc1c(NC)c(C=C)c2c(C)c(N)nc(N)c2c1C] | N-5-methyl, 2,5,8-triamine, 3,7-dimethyl, 4,6-diethenyl, isoquinoline | estimated pKa: 10.8 estimated logP (neutral/ionized): 3.3/1.3 estimated minimal projection area: 46 Å² estimated z-length: 12 Å predicted rate of energy dissipation: 79 × 10⁶ a.u. of power |
| 46-3 | 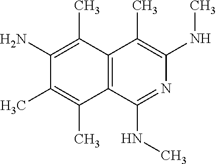 [SMILES: CNc1nc(NC)c2c(C)c(C)c(N)c(C)c2c1C] | N-2,N-8-methyl, 2,5,8- triamine, 3,4,6,7- tetramethyl, isoquinoline | estimated pKa: 10.8 estimated logP (neutral/ionized): 3.1/1.1 estimated minimal projection area: 42 Å² estimated z-length: 12 Å predicted rate of energy dissipation: 80 × 10⁶ a.u. of power |
| 52-1 | 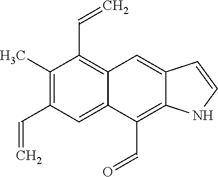 [SMILES: C=Cc1cc2c(C=O)c3[nH]ccc3cc2c(C=C)c1C] | 5,7-diethenyl-6-methyl-1H- naphto[2,3-b]pyrrole-9- carbaldehyde | estimated pKa: 11.3 estimated logP (neutral/ionized): 4.8/3.3 estimated minimal projection area: 41 Å² estimated z-length: 13 Å predicted rate of energy dissipation: 302 × 10⁶ a.u. of power |
| 52-2 | 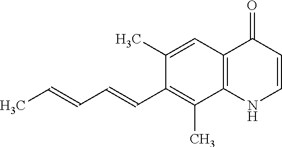 [SMILES: C/C=C/C=C/c1c(C)cc2c(=O)cc[nH]c2c1C] | 6,8-dimethyl, 7-((1E,3E)- penta-1,3-dien-1-yl), 1-H- quinolin-4-one | estimated pKa: 11.3 estimated logP (neutral/ionized): 4.6/3.1 estimated minimal projection area: 35 Å² estimated z-length: 15 Å predicted rate of energy dissipation: 390 × 10⁶ a.u. of power |

TABLE 5

| Compound | | | |
|---|---|---|---|
| Number | Chemical Structure | Chemical Name | Comments |
| 28-1 | 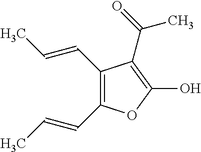 | 2-hydroxy, 3-acetyl, 4,5-di- (propen-1-yl), furan | estimated pKa: 4.6 estimated logP (neutral/ionized): 3.3/1.6 estimated minimal projection area: 36 Å² estimated z-length: 12 Å predicted rate of energy dissipation: 433 × 10⁶ a.u. of power |
| 28-2 | 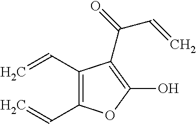 | 2-hydroxy, 3-(prop-2-en-1- one), 4,5-diethenyl, furan | estimated pKa: 4.7 estimated logP (neutral/ionized): 3.3/1.6 estimated minimal projection area: 29 Å² estimated z-length: 12 Å predicted rate of energy dissipation: 691 × 10⁶ a.u. of power |
| 29-1 | 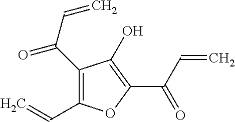 [SMILES: C=CC(=O)c1oc(C=C)c(C(=O)C=C)c1O] | 2,4-di-(prop- 2-en-1-one), 3-hydroxy, 5-ethenyl, furan | estimated pKa: 5.4 estimated logP (neutral/ionized): 3.3/1.4 estimated minimal projection area: 30 Å² estimated z-length: 14 Å predicted rate of energy dissipation: 397 × 10⁶ a.u. of power |
| 30-1 | 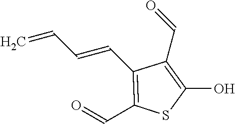 | 2-hydroxy, 3,5-diformyl, 4- [(1E)-buta- 1,3-dien-1-yl], thiofuran | estimated pKa: 4.6 estimated logP (neutral/ionized): 3.1/1.4 estimated minimal projection area: 29 Å² estimated z-length: 12 Å predicted rate of energy dissipation: 594 × 10⁶ a.u. of power |
| 30-3 | 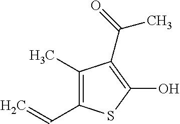 | 2-hydroxy, 3-acetyl, 4- methyl, 5-ethenyl, thiofuran | estimated pKa: 5.7 estimated logP (neutral/ionized): 3.2/1.4 estimated minimal projection area: 29 Å² estimated z-length: 11 Å predicted rate of energy dissipation: 593 × 10⁶ a.u. of power |
| 30-4 | 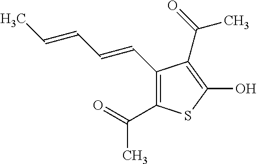 [SMILES: C/C=C/C=C/c1c(C(C)=O)sc(O)c1C(C)=O] | 2-hydroxy, 3,5-acetyl, 4- [(1E,3E)-penta- 1,3-dien-1-yl], thiofuran | estimated pKa: 4.5 estimated logP (neutral/ionized): 3.2/1.5 estimated minimal projection area: 39 Å² estimated z-length: 13 Å predicted rate of energy dissipation: 360 × 10⁶ a.u. of power |
| 31-1 | 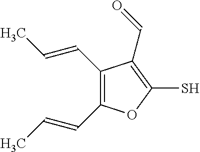 | 2-sulfanyl, 3-formyl, 4,5- di(propen-1-yl), furan | estimated pKa: 5.4 estimated logP (neutral/ionized): 3.2/1.8 estimated minimal projection area: 39 Å² estimated z-length: 11 Å predicted rate of energy dissipation: 402 × 10⁶ a.u. of power |
| 31-2 | 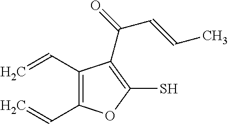 | 2-sulfanyl, 3-(but-2-en-1- one), 4,5-diethenyl, furan | estimated pKa: 5.4 estimated logP (neutral/ionized): 3.4/2.0 estimated minimal projection area: 31 Å² estimated z-length: 12 Å predicted rate of energy dissipation: 406 × 10⁶ a.u. of power |
| 31-3 | 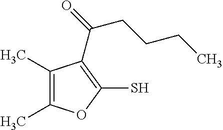 | 2-sulfanyl, 3-(pentan-1-one), 4,5-dimethyl, furan | estimated pKa: 5.6 estimated logP (neutral/ionized): 3.2/1.8 estimated minimal projection area: 35 Å² estimated z-length: 13 Å predicted rate of energy dissipation: 362 × 10⁶ a.u. of power |
| 32-1 | 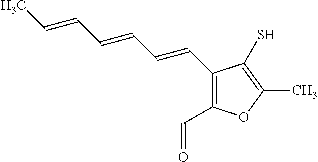 [SMILES: C/C=C/C=C/C=C/c1c(C=O)oc(C)c1S] | 2-methyl, 3-sulfanyl, 4- [(1E,3E,5E)- hepta-1,3,5-trien- 1-yl], 5-formyl, furan | estimated pKa: 5.8 estimated logP (neutral/ionized): 3.2/1.8 estimated minimal projection area: 31 Å² estimated z-length: 16 Å predicted rate of energy dissipation: 377 × 10⁶ a.u. of power |
| 32-3 | 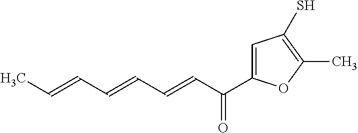 [SMILES: C/C=C/C=C/C=C/C(=O)c1cc(S)c(C)o1] | 2-methyl, 3-sulfanyl, 5- ((2E,4E,6E)- octa-2,4,6-trien- 1-one), furan | estimated pKa: 5.6 estimated logP (neutral/ionized): 3.1/1.7 estimated minimal projection area: 28 Å² estimated z-length: 17 Å predicted rate of energy dissipation: 347 × 10⁶ a.u. of power |
| 32-4 | 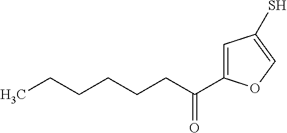 | 3-sulfanyl, 5-(heptan-1-one), furan | estimated pKa: 5.5 estimated logP (neutral/ionized): 3.2/1.7 estimated minimal projection area: 30 Å² estimated z-length: 17 Å predicted rate of energy dissipation: 397 × 10⁶ a.u. of power |
| 33-1 | 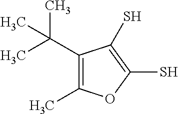 | 2,3-dithiol, 4-tert-butyl, 5- methyl, furan | estimated pKa: 5.4; 6.6 estimated logP (neutral/ionized): 3.1/1.7; 1.7; 0.3 estimated minimal projection area: 39 Å² estimated z-length: 10 Å predicted rate of energy dissipation: 915 × 10⁶ a.u. of power |
| 34-1 | 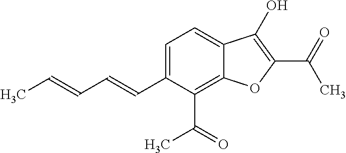 [SMILES: C/C=C/C=C/c1ccc2c(O)c(C(C)=O)oc2c1C(C)=O] | 2,7-diacetyl, 3-hydroxy, 6- ((1E,3E)-penta- 1,3-dien-1-yl), benzofuran | estimated pKa: 5.1 estimated logP (neutral/ionized): 3.2/1.4 estimated minimal projection area: 32 Å² estimated z-length: 16 Å predicted rate of energy dissipation: 379 × 10⁶ a.u. of power |
| 34-2 | 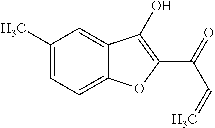 | 2-(prop-2-en- 1-one), 3- hydroxy, 5-methyl, benzofuran | estimated pKa: 6.2 estimated logP (neutral/ionized): 3.2/1.4 estimated minimal projection area: 26 Å² estimated z-length: 13 Å predicted rate of energy dissipation: 484 × 10⁶ a.u. of power |
| 34-3 | 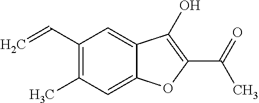 [SMILES: C=Cc1cc2c(O)c(C(C)=O)oc2cc1C] | 2-acetyl, 3-hydroxy, 5- ethenyl, 6-methyl, benzofuran | estimated pKa: 6.0 estimated logP (neutral/ionized): 3.2/1.4 estimated minimal projection area: 30 Å² estimated z-length: 13 Å predicted rate of energy dissipation: 433 × 10⁶ a.u. of power |
| 34-4 | 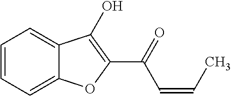 | 2-(but-2-en- 1-one), 3- hydroxy, benzofuran | estimated pKa: 5.9 estimated logP (neutral/ionized): 3.1/1.3 estimated minimal projection area: 28 Å² estimated z-length: 14 Å predicted rate of energy dissipation: 392 × 10⁶ a.u. of power |
| 34-5 | 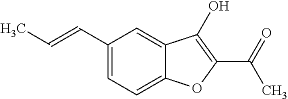 [SMILES: C/C=C/c1ccc2oc(C(C)=O)c(O)c2c1] | 2-acetyl, 3-hydroxy, 5- (propen-1-yl) benzofuran | estimated pKa: 5.9 estimated logP (neutral/ionized): 3.1/1.3 estimated minimal projection area: 27 Å² estimated z-length: 14 Å predicted rate of energy dissipation: 369 × 10⁶ a.u. of power |
| 34-6 | 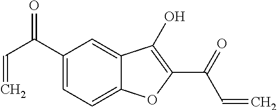 [SMILES: C=CC(=O)c1ccc2oc(C(=O)C=C)c(O)c2c1] | 2,5-di-(prop-2- en-1-one), 3- hydroxy, benzofuran | estimated pKa: 5.1 estimated logP (neutral/ionized): 3.0/1.3 estimated minimal projection area: 29 Å² estimated z-length: 15 Å predicted rate of energy dissipation: 351 × 10⁶ a.u. of power |
| 34-7 | 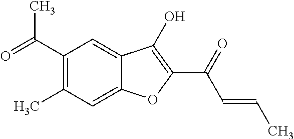 [SMILES: C/C=C/C(=O)c1oc2cc(C)c(C(C)=O)cc2c1O] | 2-(but-2-en- 1-one), 3- hydroxy, 5-acetyl, 6-methyl, benzofuran | estimated pKa: 5.1 estimated logP (neutral/ionized): 3.2/1.4 estimated minimal projection area: 34 Å² estimated z-length: 16 Å predicted rate of energy dissipation: 340 × 10⁶ a.u. of power |
| 34-8 | 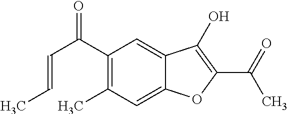 [SMILES: C/C=C/C(=O)c1cc2c(O)c(C(C)=O)oc2cc1C] | 2-acetyl, 3-hydroxy, 5-(but-2- en-1-one), 6-methyl, benzofuran | estimated pKa: 5.0 estimated logP (neutral/ionized): 3.2/1.5 estimated minimal projection area: 35 Å² estimated z-length: 15 Å predicted rate of energy dissipation: 338 × 10⁶ a.u. of power |
| 34-9 | 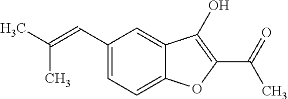 [SMILES: CC(=O)c1oc2ccc(C=C(C)C)cc2c1O] | 2-acetyl, 3-hydroxy, 5-(2- methylprop- 1-en-1-yl), benzofuran | estimated pKa: 5.9 estimated logP (neutral/ionized): 3.3/1.5 estimated minimal projection area: 30 Å² estimated z-length: 14 Å predicted rate of energy dissipation: 317 × 10⁶ a.u. of power |
| 34-10 | 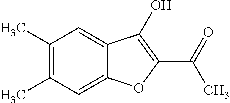 | 2-acetyl, 3-hydroxy, 5,6- dimethyl, benzofuran | estimated pKa: 6.2 estimated logP (neutral/ionized): 3.0/1.2 estimated minimal projection area: 28 Å² estimated z-length: 12 Å predicted rate of energy dissipation: 291 × 10⁶ a.u. of power |
| 34-11 | 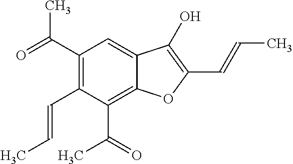 [SMILES: C/C=C/c1oc2c(C(C)=O)c(/C=C/C)c(C(C)=O)cc2c1O] | 2,6-di(propen- 1-yl), 3- hydroxy, 5,7-diacetyl, benzofuran | estimated pKa: 5.5 estimated logP (neutral/ionized): 3.1/1.6 estimated minimal projection area: 40 Å² estimated z-length: 15 Å predicted rate of energy dissipation: 224 × 10⁶ a.u. of power |
| 34-12 | 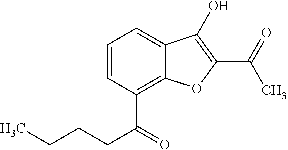 [SMILES: CCCCC(=O)c1cccc2c(O)c(C(C)=O)oc12] | 2-acetyl, 3-hydroxy, 7- (pentan-1-one), benzofuran | estimated pKa: 5.0 estimated logP (neutral/ionized): 3.1/1.4 estimated minimal projection area: 37 Å² estimated z-length: 15 Å predicted rate of energy dissipation: 261 × 10⁶ a.u. of power |
| 34-13 | 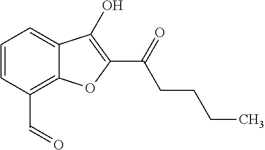 [SMILES: CCCCC(=O)c1oc2c(C=O)cccc2c1O] | 2-(pentan-1-one), 3-hydroxy, 7-formyl, benzofuran | estimated pKa: 5.0 estimated logP (neutral/ionized): 3.3/1.5 estimated minimal projection area: 35 Å² estimated z-length: 14 Å predicted rate of energy dissipation: 365 × 10⁶ a.u. of power |
| 34-14 | 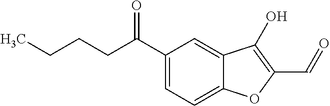 [SMILES: CCCCC(=O)c1ccc2oc(C=O)c(O)c2c1] | 2-formyl, 3-hydroxy, 5- (pentan-l-one), benzofuran | estimated pKa: 5.0 estimated logP (neutral/ionized): 3.3/1.5 estimated minimal projection area: 33 Å² estimated z-length: 16 Å predicted rate of energy dissipation: 362 × 10⁶ a.u. of power |
| 34-15 | 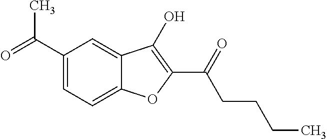 [SMILES: CCCCC(=O)c1oc2ccc(C(C)=O)cc2c1O] | 2-(pentan-1-one), 3-hydroxy, 5-acetyl, benzofuran | estimated pKa: 4.9 estimated logP (neutral/ionized): 3.1/1.4 estimated minimal projection area: 32 Å² estimated z-length: 16 Å predicted rate of energy dissipation: 332 × 10⁶ a.u. of power |
| 35-1 | 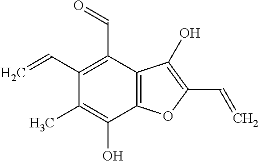 [SMILES: C=Cc1oc2c(O)c(C)c(C=C)c(C=O)c2c1O] | 2,5-diethenyl, 3,7-dihydroxy, 4-formyl, 6-methyl, benzofuran | estimated pKa: 5.5; 6.9 estimated logP (neutral/ionized): 3.1/1.6; 1.2; 0.0 estimated minimal projection area: 33 Å² estimated z-length: 13 Å predicted rate of energy dissipation: 812 × 10⁶ a.u. of power |
| 35-2 | 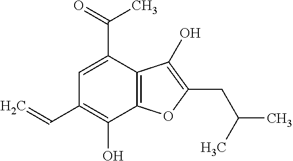 [SMILES: C=Cc1cc(C(C)=O)c2c(O)c(CC(C)C)oc2c1O] | 2-(2-methylprop- 1-en-1-yl), 3,7-dihydroxy, 4-acetyl, 6- ethenyl, benzofuran | estimated pKa: 5.3; 6.9 estimated logP (neutral/ionized): 3.1/1.6; 1.1; −0.1 estimated minimal projection area: 39 Å² estimated z-length: 14 Å predicted rate of energy dissipation: 565 × 10⁶ a.u. of power |
| 35-3 | 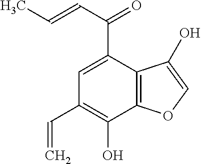 [SMILES: C=Cc1cc(C(=O)/C=C/C)c2c(O)coc2c1O] | 3,7-dihydroxy, 4-(but-2-en-1- one), 6-ethenyl, benzofuran | estimated pKa: 5.1; 6.9 estimated logP (neutral/ionized): 3.0/1.4; 1.0; −0.2 estimated minimal projection area: 37 Å² estimated z-length: 12 Å predicted rate of energy dissipation: 529 × 10⁶ a.u. of power |
| 35-4 | 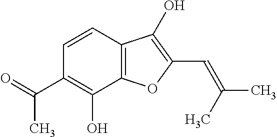 [SMILES: CC(=O)c1ccc2c(O)c(C=C(C)C)oc2c1O] | 2-(2-methylprop- 1-en-1-yl), 3,7-dihydroxy, 6-acetyl, benzofuran | estimated pKa: 5.6; 7.2 estimated logP (neutral/ionized): 3.0/1.5; 0.8; −0.4 estimated minimal projection area: 30 Å² estimated z-length: 14 Å predicted rate of energy dissipation: 497 × 10⁶ a.u. of power |
| 35-5 | 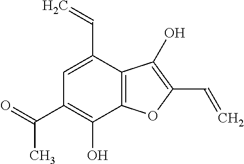 [SMILES: C=Cc1oc2c(O)c(C(C)=O)cc(C=C)c2c1O] | 2,4-diethenyl, 3,7-dihydroxy, 6-acetyl, benzofuran | estimated pKa: 5.6; 7.3 estimated logP (neutral/ionized): 3.1/1.6; 0.9; −0.3 estimated minimal projection area: 34 Å² estimated z-length: 13 Å predicted rate of energy dissipation: 544 × 10⁶ a.u. of power |
| 36-1 | 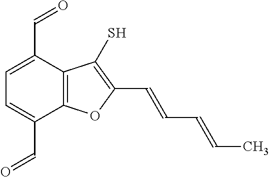 [SMILES: C/C=C/C=C/c1oc2c(C=O)ccc(C=O)c2c1S] | 2-((1E,3E)-penta- 1,3-dien-1- yl), 3-sulfanyl, 4,7-diformyl, benzofuran | estimated pKa: 5.7 estimated logP (neutral/ionized): 3.2/1.8 estimated minimal projection area: 39 Å² estimated z-length: 14 Å predicted rate of energy dissipation: 299 × 10⁶ a.u. of power |
| 36-2 | 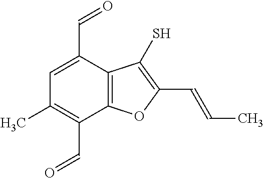 [SMILES: C/C=C/c1oc2c(C=O)c(C)cc(C=O)c2c1S] | 2-(propen-1-yl), 3-sulfanyl, 4,7-diformyl, 6-methyl, benzofuran | estimated pKa: 5.6 estimated logP (neutral/ionized): 3.2/1.8 estimated minimal projection area: 39 Å² estimated z-length: 13 Å predicted rate of energy dissipation: 313 × 10⁶ a.u. of power |
| 36-3 | 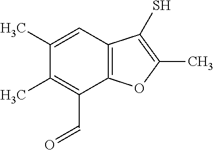 | 2,5,6-trimethyl, 3-sulfanyl, 7- formyl, benzofuran | estimated pKa: 5.9 estimated logP (neutral/ionized): 3.2/1.7 estimated minimal projection area: 31 Å² estimated z-length: 11 Å predicted rate of energy dissipation: 490 × 10⁶ a.u. of power |
| 37-1 | 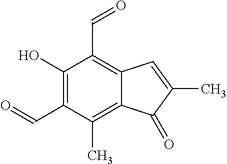 [SMILES: CC1=Cc2c(C=O)c(O)c(C=O)c(C)c2C1=O] | 2,7-dimethyl, 4,6-diformyl, 5- hydroxy, inden-1-one | estimated pKa: 5.4 estimated logP (neutral/ionized): 3.3/0.9 estimated minimal projection area: 32 Å² estimated z-length: 11 Å predicted rate of energy dissipation: 400 × 10⁶ a.u. of power |
| 37-2 | 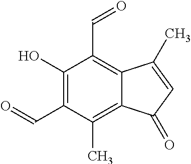 [SMILES: CC1=CC(=O)c2c(C)c(C=O)c(O)c(C=O)c21] | 3,7-dimethyl, 4,6-diformyl, 5- hydroxy, inden-1-one | estimated pKa: 5.4 estimated logP (neutral/ionized): 3.2/0.8 estimated minimal projection area: 31 Å² estimated z-length: 11 Å predicted rate of energy dissipation: 407 × 10⁶ a.u. of power |
| 37-3 | 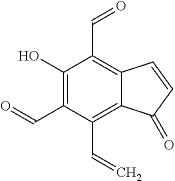 [SMILES: C=Cc1c(C=O)c(O)c(C=O)c2c1C(=O)C=C2] | 4,6-diformyl, 5-hydroxy, 7- ethenyl, inden-1-one | estimated pKa: 5.3 estimated logP (neutral/ionized): 3.1/0.7 estimated minimal projection area: 32 Å² estimated z-length: 11 Å predicted rate of energy dissipation: 353 × 10⁶ a.u. of power |
| 37-4 | 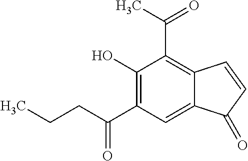 [SMILES: CCCC(=O)c1cc2c(c(C(C)=O)c1O)C=CC2=O] | 4-acetyl, 5-hydroxy, 6-(butan- 1-one), inden-1-one | estimated pKa: 5.2 estimated logP (neutral/ionized): 3.2/0.8 estimated minimal projection area: 36 Å² estimated z-length: 14 Å predicted rate of energy dissipation: 283 × 10⁶ a.u. of power |
| 38-1 | 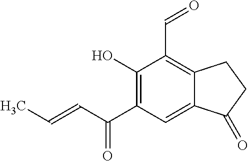 [SMILES: C/C=C/C(=O)c1cc2c(c(C=O)c1O)CCC2=O] | 4-formyl, 5-hydroxy, 6-(but- 2-en-1-one), dihydro-inden- 1-one | estimated pKa: 5.2 estimated logP (neutral/ionized): 3.3/0.9 estimated minimal projection area: 36 Å² estimated z-length: 13 Å predicted rate of energy dissipation: 290 × 10⁶ a.u. of power |
| 38-2 | 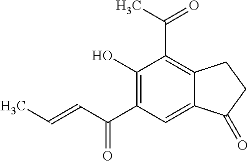 [SMILES: C/C=C/C(=O)c1cc2c(c(C(C)=O)c1O)CCC2=O] | 4-acetyl, 5-hydroxy, 6-(but-2- en-1-one), dihydro-inden-1- one | estimated pKa: 5.2 estimated logP (neutral/ionized): 3.1/0.7 estimated minimal projection area: 39 Å² estimated z-length: 13 Å predicted rate of energy dissipation: 205 × 10⁶ a.u. of power |
| 39-1 | 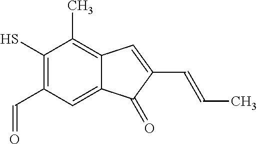 [SMILES: C/C=C/C1=Cc2c(cc(C=O)c(S)c2C)C1=O] | 2-(propen-1-yl), 4-methyl, 5- sulfanyl, 6-formyl, inden-1- one | estimated pKa: 5.8 estimated logP (neutral/ionized): 3.2/1.7 estimated minimal projection area: 32 Å² estimated z-length: 14 Å predicted rate of energy dissipation: 382 × 10⁶ a.u. of power |
| 39-2 | 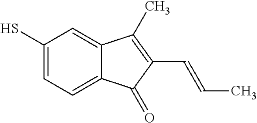 | 2-(propen-1-yl), 3-methyl, 5- sulfanyl, inden-1-one | estimated pKa: 6.1 estimated logP (neutral/ionized): 3.2/1.8 estimated minimal projection area: 30 Å² estimated z-length: 14 Å predicted rate of energy dissipation: 410 × 10⁶ a.u. of power |
| 39-3 | 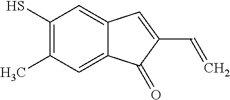 | 2-ethenyl, 5-sulfanyl, 6- methyl, inden-1-one | estimated pKa: 6.1 estimated logP (neutral/ionized): 3.1/1.6 estimated minimal projection area: 30 Å² estimated z-length: 12 Å predicted rate of energy dissipation: 354 × 10⁶ a.u. of power |
| 39-4 | 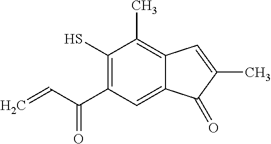 [SMILES: C=CC(=O)c1cc2c(c(C)c1S)C=C(C)C2=O] | 2,4-dimethyl, 5-sulfanyl, 6- (prop-2-en-1-one), inden-1- one | estimated pKa: 5.8 estimated logP (neutral/ionized): 3.2/1.8 estimated minimal projection area: 32 Å² estimated z-length: 13 Å predicted rate of energy dissipation: 423 × 10⁶ a.u. of power |
| 39-5 | 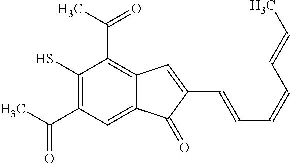 [SMILES: C/C=C/C=C\C=C\C1=Cc2c(cc(C(C)=O)c(S)c2C(C)=O)C1=O] | 2-((1E,3E,5E)- hepta-1,3,5- trien-1-yl), 4,6-diacetyl, 5- sulfanyl, inden-1-one | estimated pKa: 5.6 estimated logP (neutral/ionized): 3.1/1.7 estimated minimal projection area: 41 Å² estimated z-length: 19 Å predicted rate of energy dissipation: 158 × 10⁶ a.u. of power |
| 39-6 | 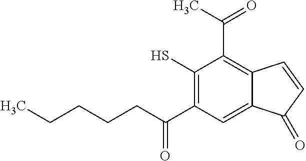 [SMILES: CCCCCC(=O)c1cc2c(c(C(C)=O)c1S)C=CC2=O] | 4-acetyl, 5-sulfanyl, 6-(hexan- 1-one), inden-1-one | estimated pKa: 5.6 estimated logP (neutral/ionized): 3.2/1.7 estimated minimal projection area: 41 Å² estimated z-length: 17 Å predicted rate of energy dissipation: 207 × 10⁶ a.u. of power |
| 39-7 | 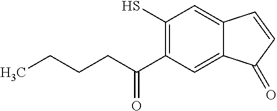 [SMILES: CCCCC(=O)c1cc2c(cc1S)C=CC2=O] | 5-sulfanyl, 6-(pentan-1-one), inden-1-one | estimated pKa: 5.8 estimated logP (neutral/ionized): 3.2/1.7 estimated minimal projection area: 35 Å² estimated z-length: 14 Å predicted rate of energy dissipation: 307 × 10⁶ a.u. of power |
| 40-1 | 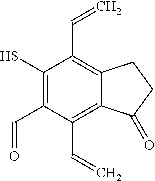 [SMILES: C=Cc1c(S)c(C=O)c(C=C)c2c1CCC2=O] | 4,7-diethenyl, 5-sulfanyl, 6- formyl, dihydro- inden-1-one | estimated pKa: 5.7 estimated logP (neutral/ionized): 3.1/1.7 estimated minimal projection area: 40 Å² estimated z-length: 10 Å predicted rate of energy dissipation: 319 × 10⁶ a.u. of power |
| 40-2 | 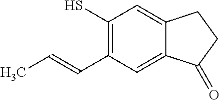 | 5-sulfanyl, 6-(propen-1-yl), dihydro- inden-1-one | estimated pKa: 6.1 estimated logP (neutral/ionized): 3.1/1.6 estimated minimal projection area: 32 Å² estimated z-length: 12 Å predicted rate of energy dissipation: 321 × 10⁶ a.u. of power |
| 40-3 | 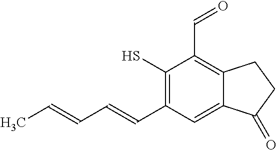 [SMILES: C/C=C/C=C/c1cc2c(c(C=O)c1S)CCC2=O] | 4-formyl, 5-sulfanyl, 6- ((1E,3E)-penta- 1,3-dien-1-yl), dihydro- inden-1-one | estimated pKa: 5.8 estimated logP (neutral/ionized): 3.3/1.9 estimated minimal projection area: 37 Å² estimated z-length: 14 Å predicted rate of energy dissipation: 264 × 10⁶ a.u. of power |
| 40-4 | 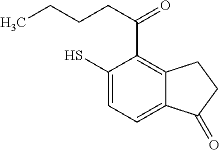 [SMILES: CCCCC(=O)c1c(S)ccc2c1CCC2=O] | 4-(pentan-1-one), 5-sulfanyl, dihydro- inden-1-one | estimated pKa: 5.9 estimated logP (neutral/ionized): 3.1/1.7 estimated minimal projection area: 35 Å² estimated z-length: 15 Å predicted rate of energy dissipation: 252 × 10⁶ a.u. of power |
| 41-1 | 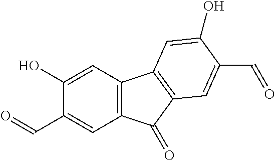 [SMILES: O=Cc1cc2c(cc1O)-c1cc(O)c(C=O)cc1C2=O] | 3,6-dihydroxy- 9-oxofluorene- 2,7-dicarbaldehyde | estimated pKa: 6.1; 6.7 estimated logP (neutral/ionized): 3.2/1.0; −1.2 estimated minimal projection area: 32 Å² estimated z-length: 14 Å predicted rate of energy dissipation: 370 × 10⁶ a.u. of power |
| 41-2 | 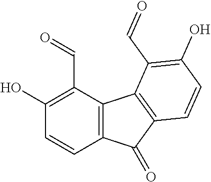 [SMILES: O=Cc1c(O)ccc2c1-c1c(ccc(O)c1C=O)C2=O] | 3,6-dihydroxy- 9-oxofluorene- 4,5-dicarbaldehyde | estimated pKa: 6.1; 6.8 estimated logP (neutral/ionized): 3.2/1.0; −1.2 estimated minimal projection area: 36 Å² estimated z-length: 12 Å predicted rate of energy dissipation: 331 × 10⁶ a.u. of power |
| 41-3 | 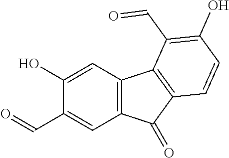 [SMILES: O=Cc1cc2c(cc1O)-c1c(ccc(O)c1C=O)C2=O] | 3,6-dihydroxy- 9-oxofluorene- 2,5-dicarbaldehyde | estimated pKa: 6.1; 6.7 estimated logP (neutral/ionized): 3.2/1.0; 1.0; −1.2 estimated minimal projection area: 34 Å² estimated z-length: 13 Å predicted rate of energy dissipation: 346 × 10⁶ a.u. of power |
| 42-1 | 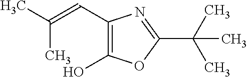 | 2-tert-butyl, 4-(2-methyl- propen-1-yl), 5-hydroxy, oxazole | estimated pKa: 5.6 estimated logP (neutral/ionized): 3.2/1.7 estimated minimal projection area: 38 Å² estimated z-length: 12 Å predicted rate of energy dissipation: 348 × 10⁶ a.u. of power |
| 42-2 | 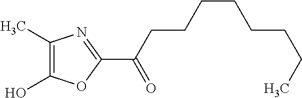 [SMILES: CCCCCCCCC(=O)c1nc(C)c(O)o1] | 2-(nonan-1-one), 4-methy, 5- hydroxy, oxazole | estimated pKa: 4.8 estimated logP (neutral/ionized): 3.1/1.6 estimated minimal projection area: 31 Å² estimated z-length: 19 Å predicted rate of energy dissipation: 377 × 10⁶ a.u. of power |
| 42-3 | 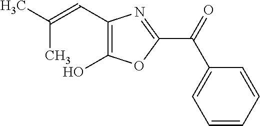 [SMILES: CC(C)=Cc1nc(C(=O)c2ccccc2)oc1O] | 2-benzoyl, 4-(2-methylprop- 1-en-1-yl), 5-hydroxy, oxazole | estimated pKa: 4.5 estimated logP (neutral/ionized): 3.1/1.7 estimated minimal projection area: 30 Å² estimated z-length: 15 Å predicted rate of energy dissipation: 560 × 10⁶ a.u. of power |
| 43-1 | 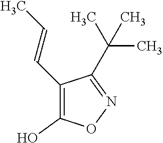 | 3-tert-butyl, 4-(propen-1-yl), 5-hydroxy, isoxazole | estimated pKa: 4.8 estimated logP (neutral/ionized): 3.2/1.7 estimated minimal projection area: 37 Å² estimated z-length: 10 Å predicted rate of energy dissipation: 538 × 10⁶ a.u. of power |
| 43-2 | 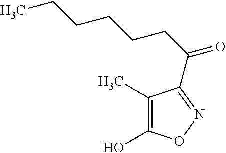 | 3-(heptan-1-one), 4-methyl, 5-hydroxy, isoxazole | estimated pKa: 4.7 estimated logP (neutral/ionized): 3.1/1.6 estimated minimal projection area: 27 Å² estimated z-length: 16 Å predicted rate of energy dissipation: 483 × 10⁶ a.u. of power |
| 52-1 | 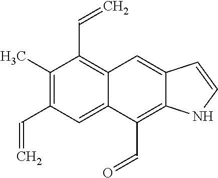 [SMILES: C=Cc1cc2c(C=O)c3[nH]ccc3cc2c(C=C)c1C] | 5,7-diethenyl- 6-methyl-1H- naphtho[2,3-b] pyrrole-9- carbaldehyde | estimated pKa: 11.3 estimated logP (neutral/ionized): 4.8/3.3 estimated minimal projection area: 41 Å² estimated z-length: 13 Å predicted rate of energy dissipation: 302 × 10⁶ a.u. of power |

TABLE 6

| Compound | | | |
|---|---|---|---|
| Number | Chemical Structure | Chemical Name | Comments |
| 25-A1 | 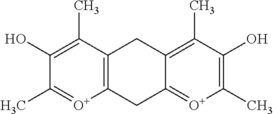 [SMILES: Cc1[o+]c2c(c(C)c1O)Cc1c([o+]c(C)c(O)c1C)C2] | 3,7-dihydroxy-2,4,6,8- tetramethyl-5H,10H- pyrano[3,2-g]chromene-1,9- bis(ylium) | estimated pKa: 6.1; 6.8 estimated logP (neutral/ionized): 3.1/1.8; 0.4 estimated minimal projection area: 35 Å² estimated z-length: 13 Å predicted rate of energy dissipation: 1216 × 10⁶ a.u. of power |
| 25-B1 | 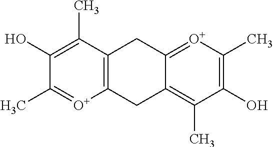 [SMILES: Cc1[o+]c2c(c(C)c1O)Cc1[o+]c(C)c(O)c(C)c1C2] | 3,8-dihydroxy-2,4,7,9- tetramethyl-5H,10H- pyrano[2,3-g]chromene-1,6- bis(ylium) | estimated pKa: 6.3; 6.9 estimated logP (neutral/ionized): 3.2/1.8; 0.5 estimated minimal projection area: 39 Å² estimated z-length: 14 Å predicted rate of energy dissipation: 1031 × 10⁶ a.u. of power |

### Pharmaceutical Compositions

The present invention also provides pharmaceutical compositions including as an active ingredient, at least one compound, preferably in a pharmacologically effective amount, more preferably in a therapeutically effective amount, suitable for any of the uses according to the present invention together with one or more pharmaceutically acceptable carriers or excipients.

The pharmaceutical composition is preferably in unit dosage form, comprising from about 0.05 mg to about 1000 mg, preferably from about 0.1 mg to about 500 mg and especially preferred from about 0.5 mg to about 200 mg of a compound suitable for any of the uses described above.

The compounds described herein may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 20^{th} Edition, Gennaro, Ed., Mack Publishing Co., Easton, Pa., 2000.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as hard or soft capsules, tablets, troches, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, aqueous or oily suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

A typical oral dosage is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from 0.05 to about 1000 mg, preferably from about 0.1 to about 500 mg, and more preferred from about 0.5 mg to about 200 mg.

For parenteral routes such as intravenous, intrathecal, intramuscular and similar administration, typically doses are in the order of about half the dose employed for oral administration.

The present invention also encompasses pharmaceutically acceptable salts of the compounds described herein. Such salts include pharmaceutically acceptable acid addition salts, pharmaceutically acceptable metal salts, ammonium, and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

Also intended as pharmaceutically acceptable acid addition salts are the hydrates which the present compounds are able to form.

The compounds described herein are generally utilized as the free substance or as a pharmaceutically acceptable salt thereof. Examples are an acid addition salt of a compound having the utility of a free base and a base addition salt of a compound having the utility of a free acid. The compounds described herein which salts are generally prepared by reacting the free base with a suitable organic or inorganic acid or by reacting the acid with a suitable organic or inorganic base. When a compound described herein contains a free base such salts are prepared in a conventional manner by treating a solution or suspension of the compound with a chemical equivalent of a pharmaceutically acceptable acid. When a compound described herein, contains a free acid such salts are prepared in a conventional manner by treating a solution or suspension of the compound with a chemical equivalent of a pharmaceutically acceptable base. Physiologically acceptable salts of a compound with a hydroxy group include the anion of said compound in combination with a suitable cation such as sodium or ammonium ion. Other salts which are not pharmaceutically acceptable may be useful in the preparation of compounds of the invention and these form a further aspect of the invention.

For parenteral administration, solutions of the compounds described herein in sterile aqueous solution, aqueous propylene glycol or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining The compounds described herein and the pharmaceutically acceptable carriers are then readily administered in a variety of dosage forms suitable for the disclosed routes of administration. The formulations may conveniently be presented in unit dosage form by methods known in the art of pharmacy.

Formulations as described herein suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

Compositions intended for oral use may be prepared according to any known method, and such compositions may contain one or more agents selected from the group consisting of sweetening agents, flavouring agents, colouring agents, and preserving agents in order to provide pharmaceutically elegant and palatable preparations. Tablets may contain the active ingredient in admixture with non-toxic pharmaceutically-accept-able excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatine or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the various techniques to form osmotic therapeutic tablets for controlled release.

Formulations for oral use may also be presented as hard gelatine capsules where the active ingredient is mixed with an inert solid diluent, for example, calcium carbonate, calcium phosphate or kaolin, or a soft gelatine capsules wherein the active ingredient is mixed with water or an oil medium, for example peanut oil, liquid paraffin, or olive oil.

Aqueous suspensions may contain the compound described herein in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavouring, and colouring agents may also be present.

The pharmaceutical compositions may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example.

For topical use, creams, ointments, jellies, solutions of suspensions, etc., containing the compounds of the invention are contemplated. For the purpose of this application, topical applications shall include mouth washes and gargles.

The compounds described herein may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

In addition, some of the compounds described herein may form solvates with water or common organic solvents. Such solvates are also encompassed within the scope of the invention.

Thus, in a further embodiment, there is provided a pharmaceutical composition including a compound described herein, or a pharmaceutically acceptable salt, solvate, or prodrug thereof, and one or more pharmaceutically acceptable carriers, excipients, or diluents.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

If desired, the pharmaceutical composition including a compound described herein may comprise a compound described herein in combination with further active substances such as those described in the foregoing.

The present invention also provides methods for the preparation of compounds. The compounds can be prepared readily according to general procedures (in which all variables are as defined before, unless so specified) using readily available starting materials, reagents and conventional synthesis procedures. In these reactions, it is also possible to make use of variants which are themselves known to those of ordinary skill in this art, but are not mentioned in greater detail.

The invention should now be illustrated with the following non-limiting examples. The chemicals were obtained from Sigma-Aldrich (St. Louis, Mo. 63178). All chemicals were of reagent grade and distilled and deionized water was used.

Unless otherwise indicated, all parts and percentages are by weight and all molecular weights are weight average molecular weight. Unless otherwise specified, all chemicals used are commercially available from, for example, Sigma-Aldrich (St. Louis, Mo.).

### EXAMPLES

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

### Preparation of Compounds

### Example 1

### Preparation of Compound 1-1 (1,3-dihydroxy, 2-(propen-1-yl), 3,6-diformyl, benzene)

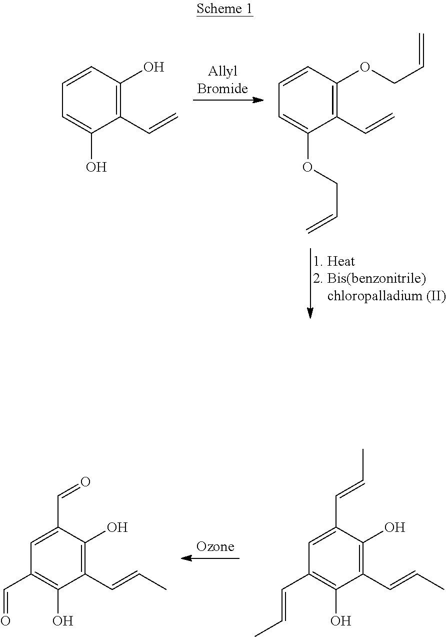

### Stage 1: Synthesis of 2-ethenyl-1,3-bis(prop-2-en-1-yloxy)benzene

1 mole of 2-ethenylbenzene-1,3-diol may be heated to reflux with 3.0 moles of allyl bromide, 2.0 moles of potassium carbonate and 0.2 moles of potassium iodide in acetone for 24 hours. The reaction mixture may be cooled to room temperature, filtered, and the solvent may be removed with a thin-film evaporator at elevated temperature and under reduced pressure. The residue may be dissolved in toluene and washed with 10% strength sodium hydroxide solution and water. The residue may be purified to afford 2-ethenyl-1,3-bis(prop-2-en-1-yloxy)benzene.

### Stage 2: Synthesis of 2,4,6-tris[(1E)-prop-1-en-1-yl]benzene-1,3-diol

1 mole of 2-ethenyl-1,3-bis(prop-2-en-1-yloxy)benzene may be heated at 230-240° C. in a metal bath for 4 hours. The reaction mixture may be cooled to room temperature and concentrated with a thin-film evaporator at elevated temperature and under reduced pressure to afford an intermediate. To 1 mole of this intermediate may be added toluene and 0.05 mole of bis(benzonitrile)dichloropalladium(II). The reaction mixture may be heated to 120° C. with stirring overnight. The reaction mixture may be cooled to room temperature, filtered through Kieselguhr, the solvent may be removed with a thin-film evaporator at elevated temperature and under reduced pressure, and the residue may purified to afford 2,4,6-tris[(1E)-prop-1-en-1-yl]benzene-1,3-diol.

### Stage 3: Synthesis of 1,3-dihydroxy, 2-(propen-1-yl), 3,6-diformyl, benzene

1 mole of 2,4,6-tris[(1E)-prop-1-en-1-yl]benzene-1,3-diol may be dissolved in dichloromethane and cooled to −60° C. To the reaction solution may be passed ozone gas from an ozone generator for 30 minutes. The reaction mixture may be treated with dimethyl sulfide, warmed to room temperature, the solvent may be removed with a thin-film evaporator at elevated temperature and under reduced pressure, and the residue may purified to afford 1,3-dihydroxy, 2-(propen-1-yl), 3,6-diformyl, benzene.

### Example 2

### Preparation of Compound 2-1 (2,4-diacetyl, 3-((1E,3E,5E)-hepta-1,3,5-trien-1-yl), thiophenol)

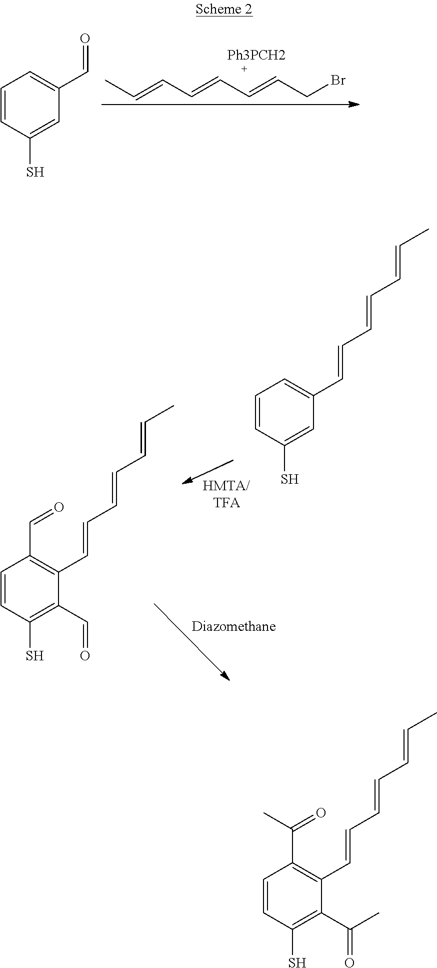

### Stage 1: Synthesis of 3-[(1E,3E,5E)-hepta-1,3,5-trien-1-yl]benzene-1-thiol

1.0 mole of 3-mercaptobenzaldehyde may be treated with 1.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 3-[(1E,3E,5E)-hepta-1,3,5-trien-1-yl]benzene-1-thiol.

### Stage 2: Synthesis of 2-[(1E,3E,5E)-hepta-1,3,5-trien-1-yl]-4-sulfanylbenzene-1,3-dicarbaldehyde

1.0 mole of 3-[(1E,3E,5E)-hepta-1,3,5-trien-1-yl]benzene-1-thiol may be treated with 1.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2-[(1E,3E,5E)-hepta-1,3,5-trien-1-yl]-4-sulfanylbenzene-1,3-dicarbaldehyde.

### Stage 3: Synthesis of 2,4-diacetyl, 3-((1E,3E,5E)-hepta-1,3,5-trien-1-yl), thiophenol

1 mole of 2-[(1E,3E,5E)-hepta-1,3,5-trien-1-yl]-4-sulfanylbenzene-1,3-dicarbaldehyde may be treated with a freshly prepared solution of 1 mole of diazomethane in tetrahydrofuran at room temperature for several hours. The solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2,4-diacetyl, 3-((1E,3E,5E)-hepta-1,3,5-trien-1-yl), thiophenol.

### Example 3

### Preparation of Compound 3-1 (2-ethenyl, 3-sulfanyl, 5-(prop-2-en-1-one), thiophenol)

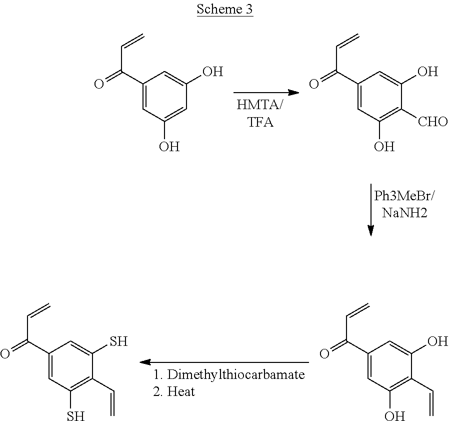

### Stage 1: Synthesis of 2,6-dihydroxy-4-(prop-2-enoyl)benzaldehyde

1 mole of 1-(3,5-dihydroxyphenyl)prop-2-en-1-one may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2,6-dihydroxy-4-(prop-2-enoyl)benzaldehyde.

### Stage 2: Synthesis of 1-(4-ethenyl-3,5-dihydroxyphenyl)prop-2-en-1-one

1.0 mole of 2,6-dihydroxy-4-(prop-2-enoyl)benzaldehyde may be treated with 1.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 1-(4-ethenyl-3,5-dihydroxyphenyl)prop-2-en-1-one.

### Stage 3: Synthesis of 2-ethenyl, 3-sulfanyl, 5-(prop-2-en-1-one), thiophenol

A solution of 0.5 mole triethylamine 0.5 mole of dimethylaminopyridine and 2 moles of N,N-dimethylthiocarbamoyl chloride may be added to a solution of 1 mole 1-(4-ethenyl-3,5-dihydroxyphenyl)prop-2-en-1-one in dioxane. The reaction mixture may be stirred for 24 hours at 100° C. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2-ethenyl, 3-sulfanyl, 5-(prop-2-en-1-one), thiophenol.

### Example 4

### Preparation of Compound 4-1 (1,3-dihydroxy, 2-(propen-1-yl), 3,6-diformyl, benzene)

Compound 4-1 was prepared according to the procedure found in the Journal of Polymer Science Part A: Polymer Chemistry, Volume 46, Issue 5, pages 1770-1782, 2008. PubChem. CID 15147116

### Example 5

### Preparation of Compound 5-1 (1,3,5-trisulfanyl, 2,4-dimethyl, 6-methoxy, benzene)

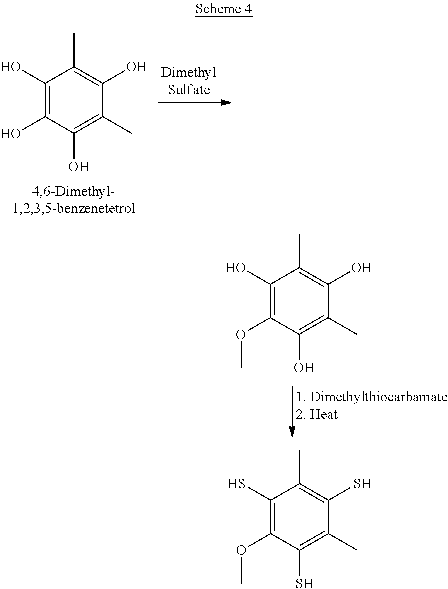

### Stage 1: Synthesis of 2-methoxy-4,6-dimethylbenzene-1,3,5-triol

1 mole of 4,6-dimethyl-1,2,3,5-benzenetetrol may be added drop wise to a solution of 1 mole of dimethyl sulfate in dioxane. The reaction mixture may be stirred at room temperature overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 2-methoxy-4,6-dimethylbenzene-1,3,5-triol.

### Stage 2: Synthesis of 1,3,5-trisulfanyl, 2,4-dimethyl, 6-methoxy, benzene

A solution of 0.5 mole triethylamine 0.5 mole of dimethylaminopyridine and 2 moles of N,N-dimethylthiocarbamoyl chloride may be added to a solution of 1 mole 2-methoxy-4,6-dimethylbenzene-1,3,5-triol in dioxane. The reaction mixture may be stirred for 24 hours at 100° C. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 1,3,5-trisulfanyl, 2,4-dimethyl, 6-methoxy, benzene.

### Example 6

### Preparation of Compound 6-1 (2-(but-3-en-2-one), 3-hydroxy, 5,7-dimethyl, chromone)

Prepared by the methods disclosed in Synthetic Communications, 17, p. 1507, 1987

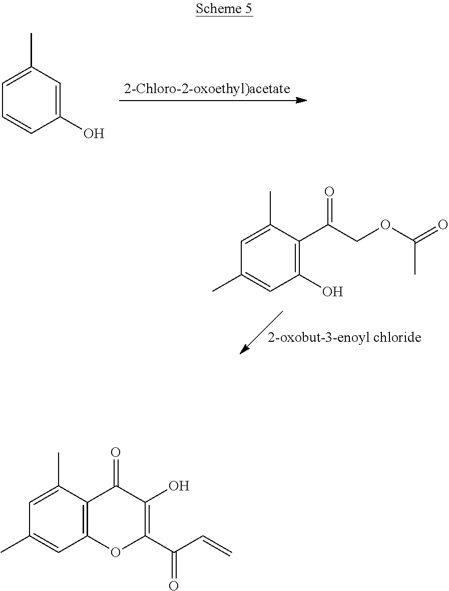

### Stage 1: Synthesis of 2,3,5-trimethylphenol 2-oxoethyl acetate

0.3 mole of aluminum chloride may be added to a solution of 1 mole freshly distilled 2-chloro-2-oxoethyl acetate in carbon disulfide at −10° C. under nitrogen atmosphere. The mixture may be stirred for fifteen minutes and warmed to room temperature. To the reaction mixture may be added 1 mole of 3,5-dimethoxyphenol drop wise with cooling. The reaction mixture may be stirred at room temperature overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 2,3,5-trimethylphenol 2-oxoethyl acetate.

### Stage 2: Synthesis of 2-(but-3-en-2-one), 3-hydroxy, 5,7-dimethyl, chromone

1 mole of 2,3,5-trimethylphenol 2-oxoethyl acetate may be dissolved in anhydrous tetrahydrofuran at room temperature. To this may be added a 1.1 mole of sodium hydride and stirred at room temperature for one hour. To this reaction mixture may be added drop wise with stirring a solution of 1 mole of 2-oxobut-3-enoyl chloride in tetrahydrofuran. The reaction mixture may be stirred at room temperature overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 2-(but-3-en-2-one), 3-hydroxy, 5,7-dimethyl, chromone.

### Example 7

### Preparation of Compound 7-1 (2,3,5-trimethyl, 6,8-diformyl, 7-hydroxy, chromone)

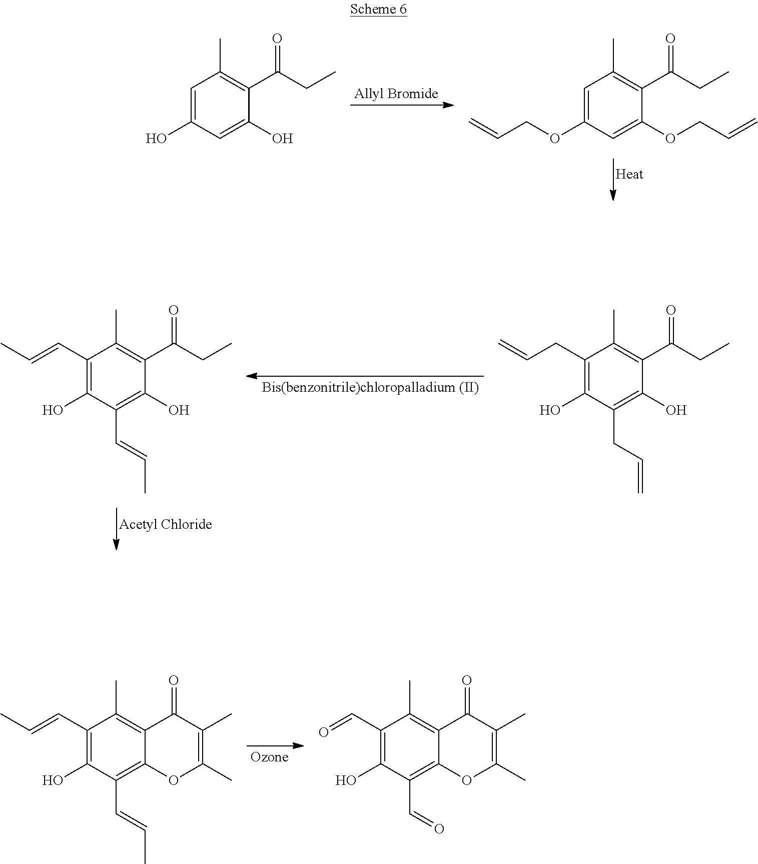 [SMILES: CCC(=O)c1c(C)cc(O)cc1O ; C=CCOc1cc(C)c(C(=O)CC)c(OCC=C)c1 ; C/C=C/c1c(C)c(C(=O)CC)c(O)c(/C=C/C)c1O ; C=CCc1c(C)c(C(=O)CC)c(O)c(CC=C)c1O ; C/C=C/c1c(O)c(/C=C/C)c2oc(C)c(C)c(=O)c2c1C ; Cc1oc2c(C=O)c(O)c(C=O)c(C)c2c(=O)c1C]

### Stage 1: Synthesis of 2-ethenyl-1,3-bis(prop-2-en-1-yloxy)benzene

1 mole of 1-(2,4-dihydroxy-6-methylphenyl)propan-1-one may be heated to reflux with 3.0 moles of allyl bromide, 2.0 moles of potassium carbonate and 0.2 moles of potassium iodide in acetone for 24 hours. The reaction mixture may be cooled to room temperature, filtered, and the solvent may be removed with a thin-film evaporator at elevated temperature and under reduced pressure. The residue may be dissolved in toluene and washed with 10% strength sodium hydroxide solution and water. The residue may be purified to afford 1-[2-methyl-4,6-bis(prop-2-en-1-yloxy)phenyl]propan-1-one.

### Stage 2: Synthesis of 1-[2,4-dihydroxy-6-methyl-3,5-bis(prop-2-en-1-yl)phenyl]propan-1-one

1 mole 1-[2-methyl-4,6-bis(prop-2-en-1-yloxy)phenyl]propan-1-one may be heated at 230-240° C. in a metal bath for 4 hours. The reaction mixture may be cooled to room temperature and concentrated with a thin-film evaporator at elevated temperature and under reduced pressure to afford 1-[2,4-dihydroxy-6-methyl-3,5-bis(prop-2-en-1-yl)phenyl]propan-1-one.

### Stage 3: Synthesis of 1-{2,4-dihydroxy-6-methyl-3,5-bis[(1E)-prop-1-en-1-yl]phenyl}propan-1-one

To 1 mole of 1-[2,4-dihydroxy-6-methyl-3,5-bis(prop-2-en-1-yl)phenyl]propan-1-one may be added toluene and 0.05 mole of bis(benzonitrile)dichloropalladium(II). The reaction mixture may be heated to 120° C. with stirring overnight. The reaction mixture may be cooled to room temperature, filtered through Kieselguhr, the solvent may be removed with a thin-film evaporator at elevated temperature and under reduced pressure, and the residue may purified to afford 1-{2,4-dihydroxy-6-methyl-3,5-bis[(1E)-prop-1-en-1-yl]phenyl}propan-1-one.

### Stage 4: Synthesis of 7-hydroxy-2,3,5-trimethyl-6,8-bis[(1E)-prop-1-en-1-yl]-4H-chromen-4-one

1 mole of 1-{2,4-dihydroxy-6-methyl-3,5-bis[(1E)-prop-1-en-1-yl]phenyl}propan-1-one may be dissolved in anhydrous tetrahydrofuran at room temperature. To this may be added a 1.1 mole of sodium hydride and stirred at room temperature for one hour. To this reaction mixture may be added drop wise with stirring a solution of 1 mole of acetyl chloride in tetrahydrofuran. The reaction mixture may be stirred at room temperature overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 7-hydroxy-2,3,5-trimethyl-6,8-bis[(1E)-prop-1-en-1-yl]-4H-chromen-4-one.

### Stage 5: Synthesis of 2,3,5-trimethyl, 6,8-diformyl, 7-hydroxy, chromone

1 mole of 7-hydroxy-2,3,5-trimethyl-6,8-bis[(1E)-prop-1-en-1-yl]-4H-chromen-4-one may be dissolved in dichloromethane and cooled to −60° C. To the reaction solution may be passed ozone gas from an ozone generator for 30 minutes. The reaction mixture may be treated with dimethyl sulfide, warmed to room temperature, the solvent may be removed with a thin-film evaporator at elevated temperature and under reduced pressure, and the residue may purified to afford 2,3,5-trimethyl, 6,8-diformyl, 7-hydroxy, chromone.

### Example 8

### Preparation of Compound 8-1 (2-ethenyl, 3,7-dihydroxy, 6-(prop-2-en-1-one), 8-methyl, chromone)

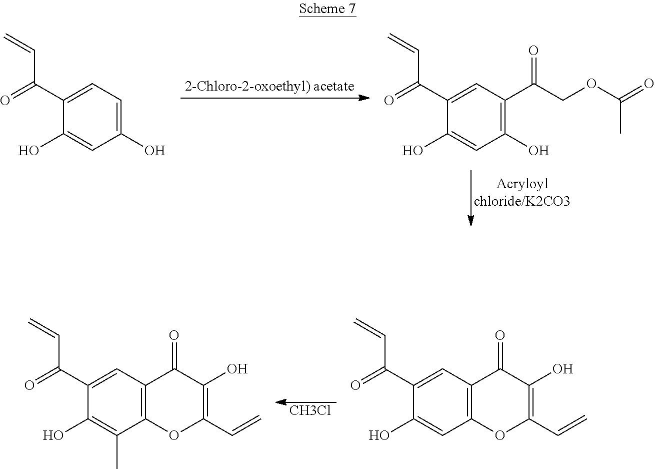

### Stage 1: Synthesis of 2-[2,4-dihydroxy-5-(prop-2-enoyl)phenyl]-2-oxoethyl acetate

0.3 mole of aluminum chloride may be added to a solution of 1 mole freshly distilled 2-chloro-2-oxoethyl acetate in carbon disulfide at −10° C. under nitrogen atmosphere. The mixture may be stirred for fifteen minutes and warmed to room temperature. To the reaction mixture may be added 1 mole of 1-(2,4-dihydroxyphenyl)prop-2-en-1-one drop wise with cooling. The reaction mixture may be stirred at room temperature overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 2-[2,4-dihydroxy-5-(prop-2-enoyl)phenyl]-2-oxoethyl acetate.

### Stage 2: Synthesis of 2-ethenyl-3,7-dihydroxy-6-(prop-2-enoyl)-4H-chromen-4-one

1 mole of 2-[2,4-dihydroxy-5-(prop-2-enoyl)phenyl]-2-oxoethyl acetate may be dissolved in anhydrous tetrahydrofuran at room temperature. To this may be added a 1.1 mole of sodium hydride and stirred at room temperature for one hour. To this reaction mixture may be added drop wise with stirring a solution of 1 mole of acryloyl chloride in tetrahydrofuran. The reaction mixture may be stirred at room temperature overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 2-ethenyl-3,7-dihydroxy-6-(prop-2-enoyl)-4H-chromen-4-one.

### Stage 3: Synthesis of 2-ethenyl, 3,7-dihydroxy, 6-(prop-2-en-1-one), 8-methyl, chromone

1 mole of 2-ethenyl-3,7-dihydroxy-6-(prop-2-enoyl)-4H-chromen-4-one may be added to a solution of 0.1 moles aluminum chloride in excess anhydrous chloromethane. The reaction mixture may be stirred at reflux overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 2-ethenyl, 3,7-dihydroxy, 6-(prop-2-en-1-one), 8-methyl, chromone.

### Example 9

### Preparation of Compound 9-1 (2,3,5-trimethyl, 6,8-diformyl, 7-hydroxy, dihydrochromone)

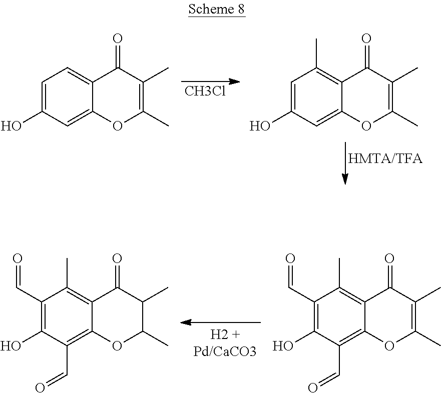

### Stage 1: Synthesis of 7-hydroxy-2,3,5-trimethyl-4H-chromen-4-one

1 mole of 7-hydroxy-2,3-dimethyl-4H-chromen-4-one may be added to a solution of 0.1 moles aluminum chloride in excess anhydrous chloromethane. The reaction mixture may be stirred at reflux overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 7-hydroxy-2,3,5-trimethyl-4H-chromen-4-one.

### Stage 2: Synthesis of 7-hydroxy-2,3,5-trimethyl-4-oxo-4H-chromene-6,8-dicarbaldehyde

1 mole of 7-hydroxy-2,3,5-trimethyl-4H-chromen-4-one may be treated with 2 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 7-hydroxy-2,3,5-trimethyl-4-oxo-4H-chromene-6,8-dicarbaldehyde.

### Stage 3: Synthesis of 2,3,5-trimethyl, 6,8-diformyl, 7-hydroxy, dihydrochromone

1 mole of 7-hydroxy-2,3,5-trimethyl-4-oxo-4H-chromene-6,8-dicarbaldehyde may be dissolved in tetrahydrofuran and a mixture of palladium and calcium carbonate may be added. The reaction mixture may be pressurized with hydrogen and sealed in a Parr hydrogenator and kept under pressure overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 2,3,5-trimethyl, 6,8-diformyl, 7-hydroxy, dihydrochromone.

### Example 10

### Preparation of Compound 10-1 (3,6-dihydroxy-9-oxoxanthene-4,5-dicarbaldehyde)

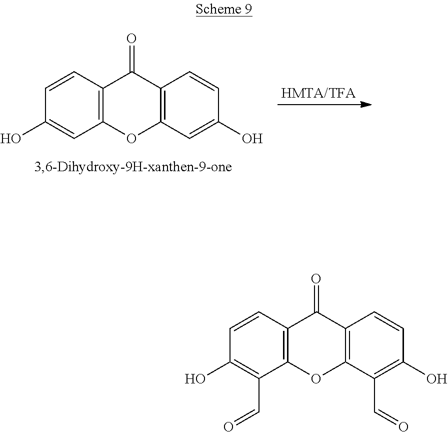 [SMILES: O=c1c2ccc(O)cc2oc2cc(O)ccc12 ; O=Cc1c(O)ccc2c(=O)c3ccc(O)c(C=O)c3oc12]

1 mole of 3,6-dihydroxy-9H-xanthen-9-one (synthesized according to the procedure on page 89. Wintner Jurgen, Ph. D. Thesis, University of Basel, 2007 may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 3,6-dihydroxy-9-oxoxanthene-4,5-dicarbaldehyde.

### Example 11

### Preparation of Compound 11-1 (2-acetyl, 3-sulfanyl, 6-((1E)-buta-1,3-dien-1-yl), chromone)

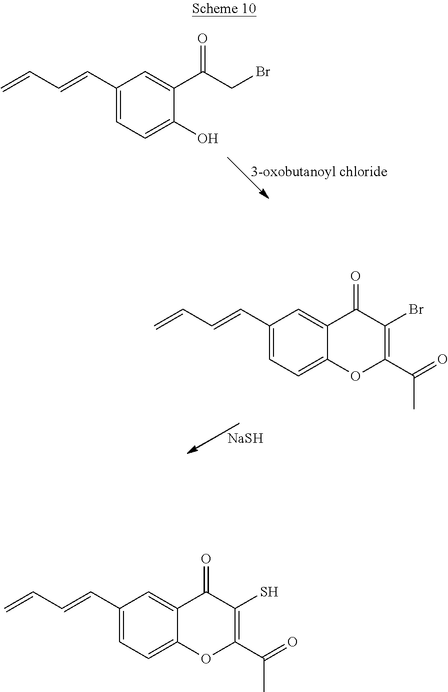 [SMILES: C=C/C=C/c1ccc(O)c(C(=O)CBr)c1 ; C=C/C=C/c1ccc2oc(C(C)=O)c(Br)c(=O)c2c1 ; "NaSH" ; C=C/C=C/c1ccc2oc(C(C)=O)c(S)c(=O)c2c1]

### Stage 1: Synthesis of 2-acetyl-3-bromo-6-[(1E)-buta-1,3-dien-1-yl]-4H-chromen-4-one

1 mole of 2-bromo-1-{5-[(1E)-buta-1,3-dien-1-yl]-2-hydroxyphenyl}ethan-1-one may be dissolved in anhydrous tetrahydrofuran at room temperature. To this may be added a 1.1 mole of sodium hydride and stirred at room temperature for one hour. To this reaction mixture may be added drop wise with stirring a solution of 1 mole of 3-oxobutanoyl chloride in tetrahydrofuran. The reaction mixture may be stirred at room temperature overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 2-acetyl-3-bromo-6-[(1E)-buta-1,3-dien-1-yl]-4H-chromen-4-one.

### Stage 2: Synthesis of 2-acetyl, 3-sulfanyl, 6-((1E)-buta-1,3-dien-1-yl), chromone

A solution of 1 mole 2-acetyl-3-bromo-6-[(1E)-buta-1,3-dien-1-yl]-4H-chromen-4-one may be added to a solution of 1 mole sodium hydrogen sulfide in dioxane. The reaction mixture may be stirred for 24 hours at 100° C. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2-acetyl, 3-sulfanyl, 6-((1E)-buta-1,3-dien-1-yl), chromone.

### Example 12

### Preparation of Compound 12-1 (2,3-diethenyl, 6-formyl, 7-sulfanyl, 8-methyl, chromone)

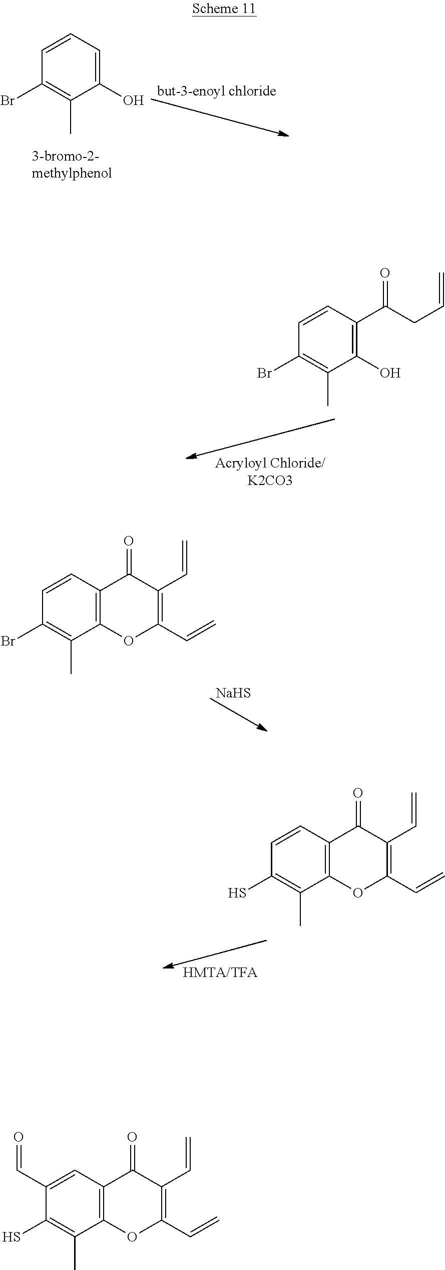

### Stage 1: Synthesis of 1-(4-bromo-2-hydroxy-3-methylphenyl)but-3-en-1-one

0.3 mole of aluminum chloride may be added to a solution of 1 mole freshly distilled but-3-enoyl chloride in carbon disulfide at −10° C. under nitrogen atmosphere. The mixture may be stirred for fifteen minutes and warmed to room temperature. To the reaction mixture may be added 1 mole of 3-bromo-2-methylphenol drop wise with cooling. The reaction mixture may be stirred at room temperature overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 1-(4-bromo-2-hydroxy-3-methylphenyl)but-3-en-1-one.

### Stage 2: Synthesis of 7-bromo-2,3-diethenyl-8-methyl-4H-chromen-4-one

1 mole of 1-(4-bromo-2-hydroxy-3-methylphenyl)but-3-en-1-one may be dissolved in anhydrous tetrahydrofuran at room temperature. To this may be added a 1.1 mole of sodium hydride and stirred at room temperature for one hour. To this reaction mixture may be added drop wise with stirring a solution of 1 mole of acryloyl chloride and potassium carbonate in tetrahydrofuran. The reaction mixture may be stirred at room temperature overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 7-bromo-2,3-diethenyl-8-methyl-4H-chromen-4-one.

### Stage 3: Synthesis of 2,3-diethenyl-8-methyl-7-sulfanyl-4H-chromen-4-one

A solution of 1 mole 7-bromo-2,3-diethenyl-8-methyl-4H-chromen-4-onemay be added to a solution of 1 mole sodium hydrogen sulfide in dioxane. The reaction mixture may be stirred for 24 hours at 100° C. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2,3-diethenyl-8-methyl-7-sulfanyl-4H-chromen-4-one.

### Stage 4: Synthesis of 2,3-diethenyl, 6-formyl, 7-sulfanyl, 8-methyl, chromone

1 mole of 2,3-diethenyl, 6-formyl, 7-sulfanyl, 8-methyl, chromone may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2,3-diethenyl, 6-formyl, 7-sulfanyl, 8-methyl, chromone.

### Example 13

### Preparation of Compound 13-1 (6-formyl, 7-sulfanyl, 8-((1E,3E,5E)-hepta-1,3,5-trien-1-yl), dihydrochromone)

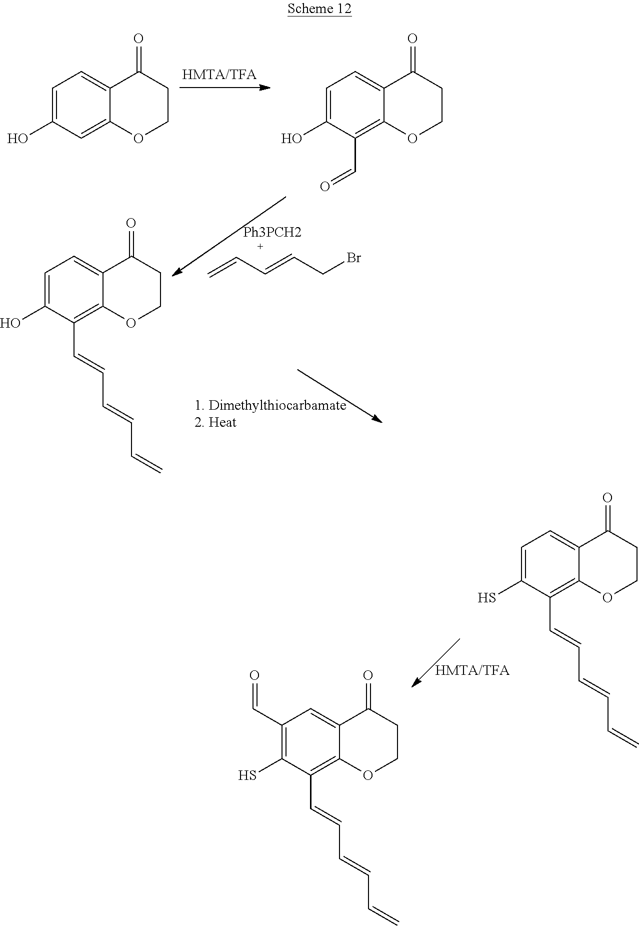

### Stage 1: Synthesis of 7-hydroxy-4-oxo-3,4-dihydro-2H-1-benzopyran-8-carbaldehyde

1 mole of 7-hydroxy-3,4-dihydro-2H-1-benzopyran-4-one hydrate may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 7-hydroxy-4-oxo-3,4-dihydro-2H-1-benzopyran-8-carbaldehyde.

### Stage 2: Synthesis of 8-[(1E,3E)-hexa-1,3,5-trien-1-yl]-7-hydroxy-3,4-dihydro-2H-1-benzopyran-4-one

1.0 mole of 7-hydroxy-4-oxo-3,4-dihydro-2H-1-benzopyran-8-carbaldehyde may be treated with 1.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 8-[(1E,3E)-hexa-1,3,5-trien-1-yl]-7-hydroxy-3,4-dihydro-2H-1-benzopyran-4-one.

### Stage 3: Synthesis of 8-[(1E,3E)-hexa-1,3,5-trien-1-yl]-7-sulfanyl-3,4-dihydro-2H-1-benzopyran-4-one

A solution of 0.5 mole triethylamine 0.5 mole of dimethylaminopyridine and 1 mole of N,N-dimethylthiocarbamoyl chloride may be added to a solution of 1 mole 8-[(1E,3E)-hexa-1,3,5-trien-1-yl]-7-hydroxy-3,4-dihydro-2H-1-benzopyran-4-one in dioxane. The reaction mixture may be stirred for 24 hours at 100° C. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 8-[(1E,3E)-hexa-1,3,5-trien-1-yl]-7-sulfanyl-3,4-dihydro-2H-1-benzopyran-4-one.

### Stage 4: Synthesis of 6-formyl, 7-sulfanyl, 8-((1E,3E,5E)-hepta-1,3,5-trien-1-yl), dihydrochromone

1 mole of 8-[(1E,3E)-hexa-1,3,5-trien-1-yl]-7-sulfanyl-3,4-dihydro-2H-1-benzopyran-4-one may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 6-formyl, 7-sulfanyl, 8-((1E,3E,5E)-hepta-1,3,5-trien-1-yl), dihydrochromone.

### Example 14

### Preparation of Compound 14-1 (2,3,8-trimethyl, 5,7-diformyl, 6-hydroxy, 1,4-naphtoquinone)

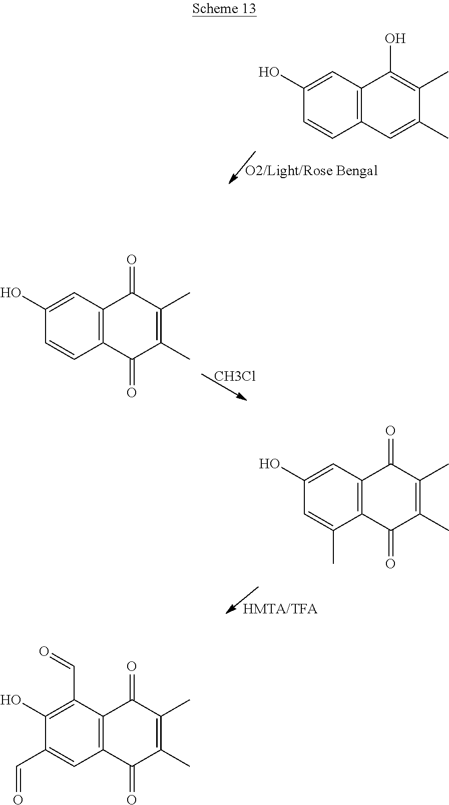

### Stage 1: Synthesis of 6-hydroxy-2,3-dimethyl-1,4-dihydronaphthalene-1,4-dione

1 mole of 2,3-dimethylnaphthalene-1,7-diol may be dissolved in tetrahydrofuran containing 0.05 mole Rose Bengal. The reaction solution may be stirred at room temperature under a bright light while oxygen may be passed through the solution overnight. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 6-hydroxy-2,3-dimethyl-1,4-dihydronaphthalene-1,4-dione

### Stage 2: Synthesis of 7-hydroxy-2,3,5-trimethyl-1,4-dihydronaphthalene-1,4-dione

1 mole of 6-hydroxy-2,3-dimethyl-1,4-dihydronaphthalene-1,4-dione may be treated with a freshly prepared solution of 1 mole of diazomethane in tetrahydrofuran at room temperature for several hours. The solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 7-hydroxy-2,3,5-trimethyl-1,4-dihydronaphthalene-1,4-dione.

### Stage 3: Synthesis of 2,3,8-trimethyl, 5,7-diformyl, 6-hydroxy, 1,4-naphtoquinone

1 mole of 7-hydroxy-2,3,5-trimethyl-1,4-dihydronaphthalene-1,4-dione may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2,3,8-trimethyl, 5,7-diformyl, 6-hydroxy, 1,4-naphtoquinone.

### Example 15

### Preparation of Compound 15-1 (1,3-diacetyl, 2-hydroxy, anthraquinone)

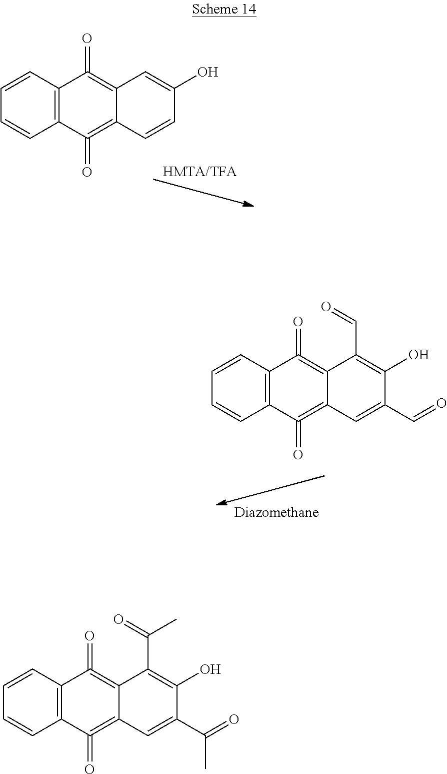

### Stage 1: Synthesis of 2-hydroxy-9,10-dioxoanthracene-1,3-dicarbaldehyde

1 mole of 2-hydroxyanthracene-9,10-dione may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2-hydroxy-9,10-dioxoanthracene-1,3-dicarbaldehyde.

### Stage 2: Synthesis of 1,3-diacetyl, 2-hydroxy, anthraquinone

1 mole of 2-hydroxy-9,10-dioxoanthracene-1,3-dicarbaldehyde may be treated with a freshly prepared solution of 1 mole of diazomethane in tetrahydrofuran at room temperature for several hours. The solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 1,3-diacetyl, 2-hydroxy, anthraquinone.

### Example 16

### Preparation of Compound 16-1 (2,6-dihydroxy, 3,7-diformyl, anthraquinone)

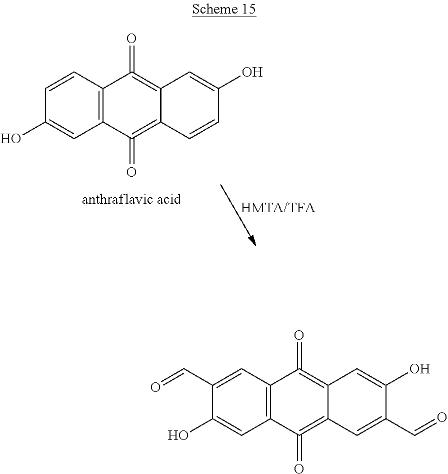

1 mole of anthraflavic acid may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2,6-dihydroxy, 3,7-diformyl, anthraquinone.

### Example 17

### Preparation of Compound 17-1 (2,3,5,8-tetramethyl, 6-sulfanyl, 7-formyl, 1,4-naphtoquinone)

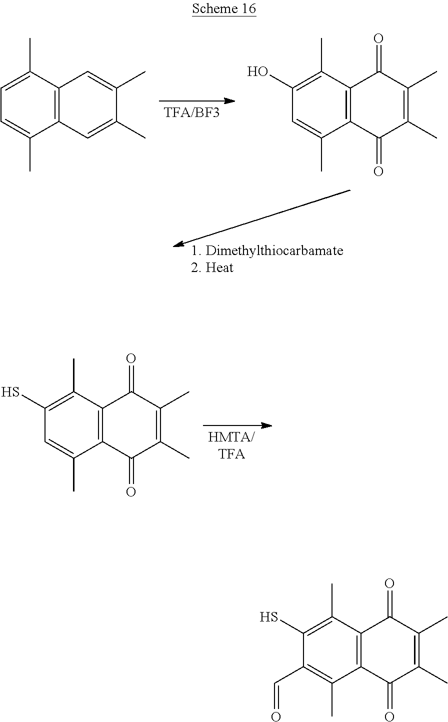

### Stage 1: Synthesis of 6-hydroxy-2,3,5,8-tetramethyl-1,4-dihydronaphthalene-1,4-dione

1 mole of 1,4,6,7-tetramethylnaphthalene may be dissolved in a solution of trifluoroacetic acid and 0.2 mole boron triflouride and stirred at reflux overnight. The reaction may be cooled to room temperature and the solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 6-hydroxy-2,3,5,8-tetramethyl-1,4-dihydronaphthalene-1,4-dione.

### Stage 2: Synthesis of 2,3,5,8-tetramethyl-6-sulfanyl-1,4-dihydronaphthalene-1,4-dione

A solution of 0.5 mole triethylamine 0.5 mole of dimethylaminopyridine and 1 mole of N,N-dimethylthiocarbamoyl chloride may be added to a solution of 1 mole 6-hydroxy-2,3,5,8-tetramethyl-1,4-dihydronaphthalene-1,4-dione in dioxane. The reaction mixture may be stirred for 24 hours at 100° C. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2,3,5,8-tetramethyl-6-sulfanyl-1,4-dihydronaphthalene-1,4-dione

### Stage 3: Synthesis of 2,3,5,8-tetramethyl, 6-sulfanyl, 7-formyl, 1,4-naphtoquinone

1 mole of 2,3,5,8-tetramethyl-6-sulfanyl-1,4-dihydronaphthalene-1,4-dione may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2,3,5,8-tetramethyl, 6-sulfanyl, 7-formyl, 1,4-naphtoquinone.

### Example 18

### Preparation of Compound 18-1 (2-sulfanyl, anthracene-9,10-dione)

95 g of 1-amino-2-methyl-4-bromoanthraquinone and 41 g of anhydrous potassium carbonate is introduced into 500 cc of dimethylformamide, 55 g of 4-tert.-butylthiophenol is added to the mixture, and the latter is heated to 125-130° C. in the course of 1 hour and is kept at this temperature until, the reaction is complete. After cooling to approx. 70° C., the reaction mixture is diluted with 500 cc of methanol and is allowed to cool completely. The dyestuff which has crystallized out is then filtered off with suction, washed with methanol and hot water and dried at 60° C. This gives 66 g of 1-amino-2-methyl-4-(4-tert.-butylphenylmercapto)-anthraquinone, which is recrystallized from dimethylformamide to remove a blue by-product. PubChem. CID 22058815; CAS 13354-38-6

### Example 19

### Preparation of Compound 19-1 (3-hydroxy, 6,7-dimethyl, chromenylium)

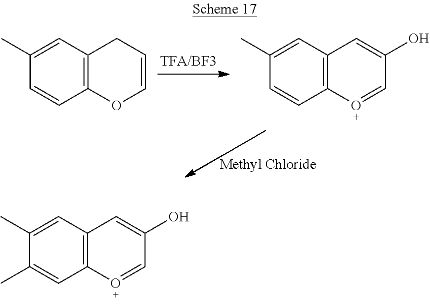

### Stage 1: Synthesis of 3-hydroxy-6-methyl-1-chromen-1-ylium

1 mole of 6-methyl-4H-chromene may be dissolved in a solution of trifluoroacetic acid and 0.2 mole boron triflouride and stirred at reflux overnight. The reaction may be cooled to room temperature and the solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 3-hydroxy-6-methyl-1-chromen-1-ylium.

### Stage 2: Synthesis of 3-hydroxy, 6,7-dimethyl, chromenylium

1 mole of 3-hydroxy-6-methyl-1$|̂{4}-chromen-1-ylium may be added to a solution of 0.1 moles aluminum chloride in excess anhydrous chloromethane. The reaction mixture may be stirred at reflux overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 3-hydroxy, 6,7-dimethyl, chromenylium.

### Example 20

### Preparation of Compound 20-1 (2-(propen-1-yl), 4-hydroxy, chromenylium)

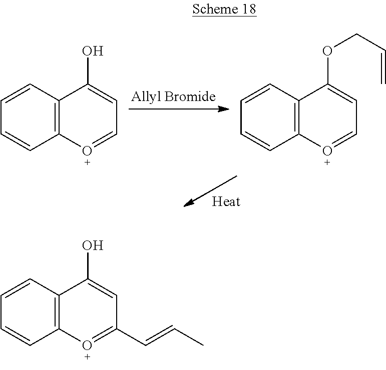

### Stage 1: Synthesis of 4-(prop-2-en-1-yloxy)-1-chromen-1-ylium

1 mole of 4-hydroxy-1-chromen-1-ylium may be heated to reflux with 1.5 moles of allyl bromide, 2.0 moles of potassium carbonate and 0.2 moles of potassium iodide in acetone for 24 hours. The reaction mixture may be cooled to room temperature, filtered, and the solvent may be removed with a thin-film evaporator at elevated temperature and under reduced pressure. The residue may be dissolved in toluene and washed with 10% strength sodium hydroxide solution and water. The residue may be purified to afford 4-(prop-2-en-1-yloxy)-1-chromen-1-ylium.

### Stage 2: Synthesis of 2-(propen-1-yl), 4-hydroxy, chromenylium

1 mole of 4-(prop-2-en-1-yloxy)-1-chromen-1-ylium may be heated at 230-240° C. in a metal bath for 4 hours. The reaction mixture may be cooled to room temperature and concentrated with a thin-film evaporator at elevated temperature and under reduced pressure to afford 2-(propen-1-yl), 4-hydroxy, chromenylium.

### Example 21

### Preparation of Compound 21-1 (7-ethenyl, 8-hydroxy, chromenylium)

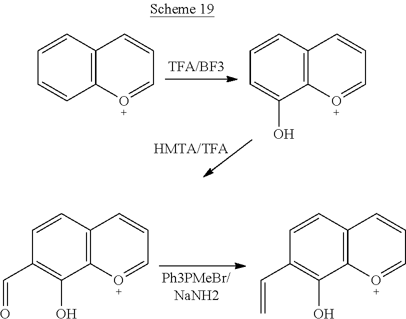

### Stage 1: Synthesis of 8-hydroxy-chromen-1-ylium

1 mole of chromen-1-ylium may be dissolved in a solution of trifluoroacetic acid and 0.2 mole boron triflouride and stirred at reflux overnight. The reaction may be cooled to room temperature and the solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 8-hydroxy-chromen-1-ylium.

### Stage 2: Synthesis of 7-formyl-8-hydroxy-chromen-1-ylium

1 mole of 8-hydroxy-chromen-1-ylium may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford formyl-8-hydroxy-chromen-1-ylium.

### Stage 3: Synthesis of 7-ethenyl, 8-hydroxy, chromenylium

1.0 mole of formyl-8-hydroxy-chromen-1-ylium may be treated with 1.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 7-ethenyl, 8-hydroxy, chromenylium.

### Example 22

### Preparation of Compound 22-1 (3,6-dihydroxy, 5-methyl, 7-ethenyl, chromenylium)

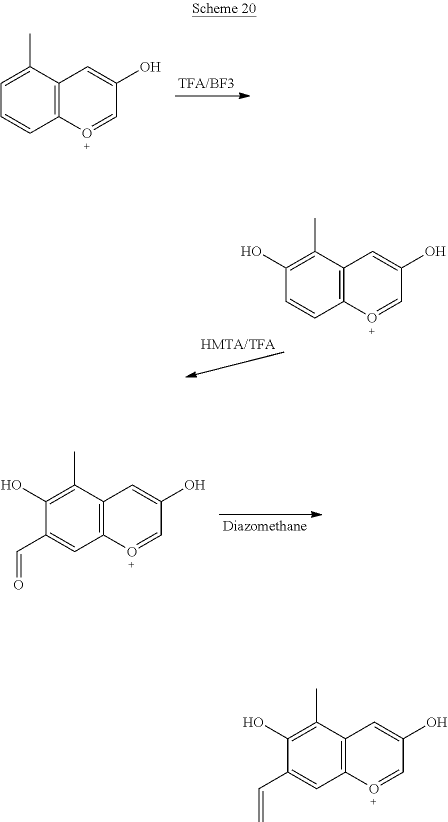

### Stage 1: Synthesis of 3,6-dihydroxy-5-methyl-chromen-1-ylium

1 mole of 3-hydroxy-5-methyl-chromen-1-ylium may be dissolved in a solution of trifluoroacetic acid and 0.2 mole boron triflouride and stirred at reflux overnight. The reaction may be cooled to room temperature and the solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 3,6-dihydroxy-5-methyl-chromen-1-ylium.

### Stage 2: Synthesis of 7-formyl-3,6-dihydroxy-5-methyl-chromen-1-ylium

1 mole of 3,6-dihydroxy-5-methyl-chromen-1-ylium may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 7-formyl-3,6-dihydroxy-5-methyl-chromen-1-ylium.

### Stage 3: Synthesis of 3,6-dihydroxy, 5-methyl, 7-ethenyl, chromenylium

1 mole of 7-formyl-3,6-dihydroxy-5-methyl-chromen-1-ylium may be treated with a freshly prepared solution of 1 mole of diazomethane in tetrahydrofuran at room temperature for several hours. The solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 3,6-dihydroxy, 5-methyl, 7-ethenyl, chromenylium.

### Example 23

### Preparation of Compound 23-1 (3-hydroxy, 4-[(1E)-buta-1,3-dien-1-yl], 5-methyl, pyrilium)

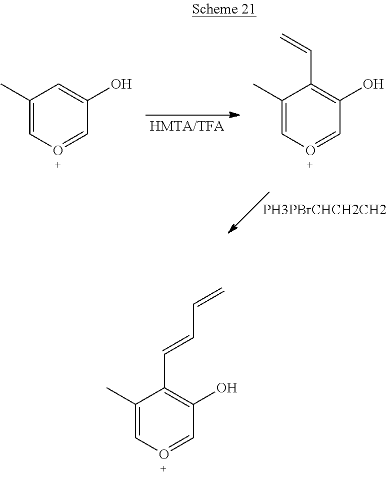

### Stage 1: Synthesis of 4-formyl-3-hydroxy-5-methyl-pyran-1-ylium

1 mole of 3-hydroxy-5-methyl-pyran-1-ylium may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 4-formyl-3-hydroxy-5-methyl-pyran-1-ylium.

### Stage 2: Synthesis of 3-hydroxy, 4-[(1E)-buta-1,3-dien-1-yl], 5-methyl, pyrilium

1.0 mole of 4-formyl-3-hydroxy-5-methyl-pyran-1-ylium may be treated with 1.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 3-hydroxy, 4-[(1E)-buta-1,3-dien-1-yl], 5-methyl, pyrilium.

### Example 24

### Preparation of Compound 24-1 (2,6-diethenyl, 3,5-diformyl, 4-hydroxy, pyrilium)

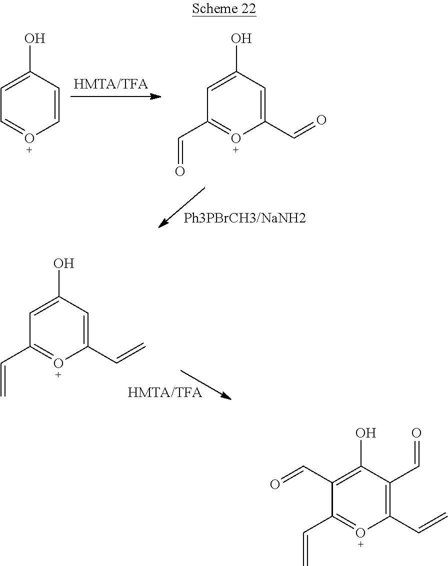

### Stage 1: Synthesis of 2,6-diformyl-4-hydroxy-pyran-1-ylium

1 mole of 4-hydroxy-pyran-1-ylium may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2,6-diformyl-4-hydroxy-pyran-1-ylium.

### Stage 2: Synthesis of 2,6-diethenyl-4-hydroxy-pyran-1-ylium

1.0 mole of 2,6-diformyl-4-hydroxy-pyran-1-ylium may be treated with 2.4 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2,6-diethenyl-4-hydroxy-pyran-1-ylium.

### Stage 3: Synthesis of 2,6-diethenyl, 3,5-diformyl, 4-hydroxy, pyrilium

1 mole of 2,6-diethenyl-4-hydroxy-pyran-1-ylium may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2,6-diethenyl, 3,5-diformyl, 4-hydroxy, pyrilium.

### Example 25

### Preparation of Compound 25-A1 (3,7-dihydroxy-2,4,6,8-tetramethyl-5H,10H-pyrano[3,2-g]chromene-1,9-bis(ylium)

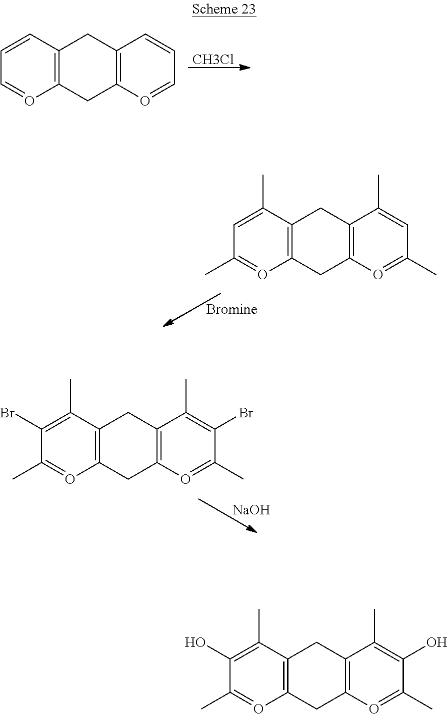

### Stage 1: Synthesis of 2,4,6,8-tetramethyl-5H,10H-pyrano[3,2-g]chromen

1 mole of 5H,10H-pyrano[3,2-g]chromone may be added to a solution of 0.1 moles aluminum chloride in excess anhydrous chloromethane. The reaction mixture may be stirred at reflux overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 2,4,6,8-tetramethyl-5H,10H-pyrano[3,2-g]chromen.

### Stage 2: Synthesis of 3,7-dibromo-2,4,6,8-tetramethyl-5H,10H-pyrano[3,2-g]chromene

1 mole of 2,4,6,8-tetramethyl-5H,10H-pyrano[3,2-g]chromen in water may be treated with two moles of bromine and stirred at room temperature overnight. The reaction mixture may be washed with dichloromethane and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford-3,7-dibromo-2,4,6,8-tetramethyl-5H,10H-pyrano[3,2-g]chromone.

### Stage 3: Synthesis of 3,7-dihydroxy-2,4,6,8-tetramethyl-5H,10H-pyrano[3,2-g]chromene-1,9-bis(ylium

1 mole of 3,7-dibromo-2,4,6,8-tetramethyl-5H,10H-pyrano[3,2-g]chromone in water may be treated with two moles of sodium hydroxide and stirred at room temperature overnight. The reaction mixture may be washed with dichloromethane and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 3,7-dihydroxy-2,4,6,8-tetramethyl-5H,10H-pyrano[3,2-g]chromene-1,9-bis(ylium).

### Example 26

### Preparation of Compound 26-1 (2-ethyl-4,5-dihydroxy-3,6,7-trimethyl-pyrano[2,3-b]pyran-1,8-bis(ylium))

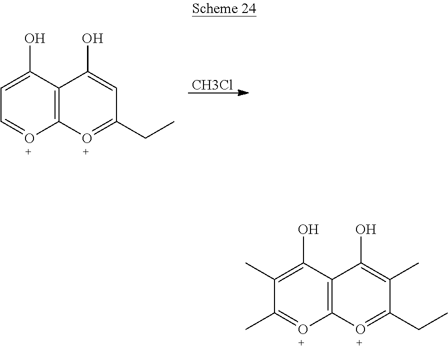

1 mole of 7-ethyl-4,5-dihydroxypyrano[2,3-b]pyran-1,8-bis(ylium) may be added to a solution of 0.1 moles aluminum chloride in excess anhydrous chloromethane. The reaction mixture may be stirred at reflux overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 2-ethyl-4,5-dihydroxy-3,6,7-trimethyl-pyrano[2,3-b]pyran-1,8-bis(ylium))

### Example 27

### Preparation of Compound 27-1 (2,4-dimethyl, 3-hydroxy, 5-[(1E,3E,5E,7E)-nona-1,3,5,7-tetraen-1-yl], 6-formyl, thiopyran)

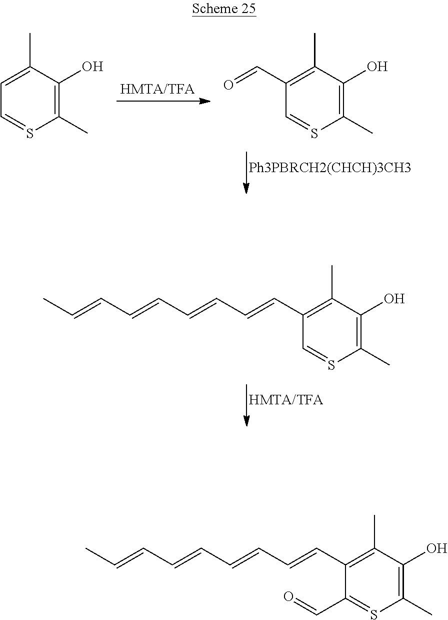

### Stage 1: Synthesis of 5-formyl-3-hydroxy-2,4-dimethyl-thiopyran-1-yl

1 mole of 3-hydroxy-2,4-dimethyl-thiopyran-1-yl may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 5-formyl-3-hydroxy-2,4-dimethyl-thiopyran-1-yl.

### Stage 2: Synthesis of 3-hydroxy-2-methyl-5-[(1E,3E,5E,7E)-nona-1,3,5,7-tetraen-1-yl]-thiopyran-1-yl

1.0 mole of 5-formyl-3-hydroxy-2,4-dimethyl-thiopyran-1-yl may be treated with 1.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 3-hydroxy-2-methyl-5-[(1E,3E,5E,7E)-nona-1,3,5,7-tetraen-1-yl]-thiopyran-1-yl.

### Stage 3: Synthesis of 2,4-dimethyl, 3-hydroxy, 5-[(1E,3E,5E,7E)-nona-1,3,5,7-tetraen-1-yl], 6-formyl, thiopyran

1 mole of 3-hydroxy-2-methyl-5-[(1E,3E,5E,7E)-nona-1,3,5,7-tetraen-1-yl]-thiopyran-1-yl may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2,4-dimethyl, 3-hydroxy, 5-[(1E,3E,5E,7E)-nona-1,3,5,7-tetraen-1-yl], 6-formyl, thiopyran.

### Example 28

### Preparation of Compound 28-1 (2-hydroxy, 3-acetyl, 4,5-di-(propen-1-yl), furan)

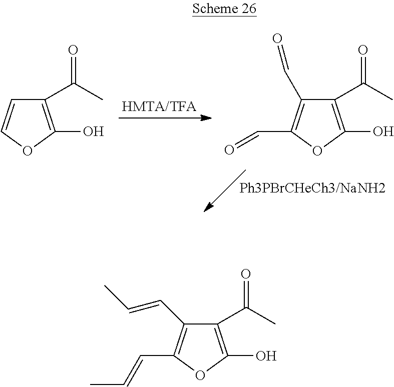

### Stage 1: Synthesis of 4-acetyl-5-hydroxyfuran-2,3-dicarbaldehyde

1 mole of 4-acetyl-5-hydroxyfuran may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 4-acetyl-5-hydroxyfuran-2,3-dicarbaldehyde.

### Stage 2: Synthesis of 2-hydroxy, 3-acetyl, 4,5-di-(propen-1-yl), furan

1.0 mole of 4-acetyl-5-hydroxyfuran-2,3-dicarbaldehyde may be treated with 2.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2-hydroxy, 3-acetyl, 4,5-di-(propen-1-yl), furan.

### Example 29

### Preparation of Compound 29-1 (2,4-di-(prop-2-en-1-one), 3-hydroxy, 5-ethenyl, furan)

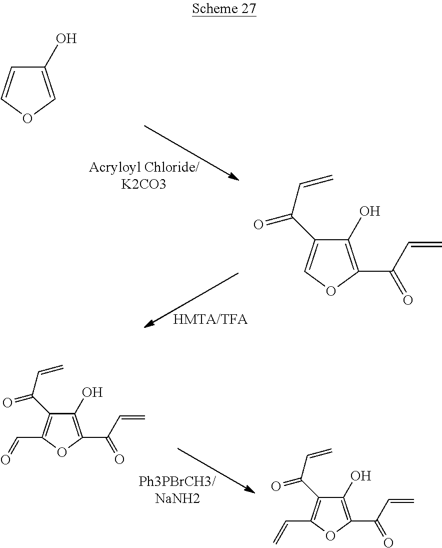 [SMILES: Oc1ccoc1 ; C=CC(=O)c1coc(C(=O)C=C)c1O ; C=CC(=O)c1oc(C=O)c(C(=O)C=C)c1O ; C=CC(=O)c1oc(C=C)c(C(=O)C=C)c1O]

### Stage 1: Synthesis of 1-[3-hydroxy-4-(prop-2-enoyl)furan-2-yl]prop-2-en-1-one

0.3 mole of aluminum chloride may be added to a solution of 1 mole freshly distilled 3-hydroxyfuran in carbon disulfide at −10° C. under nitrogen atmosphere. The mixture may be stirred for fifteen minutes and warmed to room temperature. To the reaction mixture may be added 1 moles of acryloyl chloride drop wise with cooling. The reaction mixture may be stirred at room temperature overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 1-[3-hydroxy-4-(prop-2-enoyl)furan-2-yl]prop-2-en-1-one.

### Stage 2: Synthesis of 4-hydroxy-3,5-bis(prop-2-enoyl)furan-2-carbaldehyde

1 mole of 1-[3-hydroxy-4-(prop-2-enoyl)furan-2-yl]prop-2-en-1-one may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 4-hydroxy-3,5-bis(prop-2-enoyl)furan-2-carbaldehyde.

### Stage 3: Synthesis of 2,4-di-(prop-2-en-1-one), 3-hydroxy, 5-ethenyl, furan

1.0 mole of 4-hydroxy-3,5-bis(prop-2-enoyl)furan-2-carbaldehyde may be treated with 1.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2,4-di-(prop-2-en-1-one), 3-hydroxy, 5-ethenyl, furan.

### Example 30

### Preparation of Compound 30-1 (2-hydroxy, 3,5-diformyl, 4-[(1E)-buta-1,3-dien-1-yl], thiofuran)

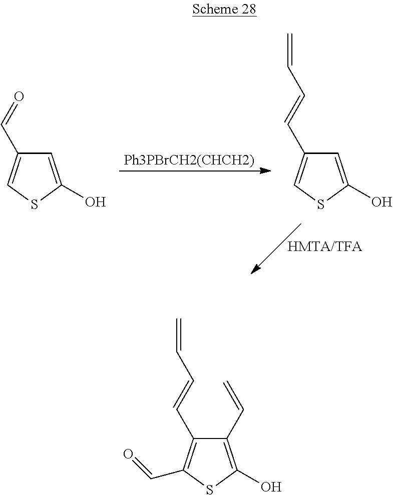

### Stage 1: Synthesis of 4-[(1E)-buta-1,3-dien-1-yl]thiophen-2-ol

1.0 mole of 5-hydroxythiophene-3-carbaldehyde may be treated with 1.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 4-[(1E)-buta-1,3-dien-1-yl]thiophen-2-ol.

### Stage 2: Synthesis of 2-hydroxy, 3,5-diformyl, 4-[(1E)-buta-1,3-dien-1-yl], thiofuran

1 mole of 4-[(1E)-buta-1,3-dien-1-yl]thiophen-2-ol may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2-hydroxy, 3,5-diformyl, 4-[(1E)-buta-1,3-dien-1-yl], thiofuran.

### Example 31

### Preparation of Compound 31-1 (2-sulfanyl, 3-formyl, 4,5-di(propen-1-yl), furan)

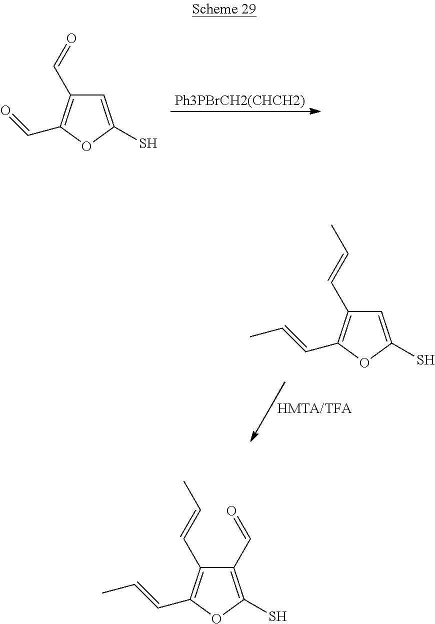

### Stage 1: Synthesis of 4,5-bis[(1E)-prop-1-en-1-yl]furan-2-thiol

1.0 mole of 5-sulfanylfuran-2,3-dicarbaldehyde may be treated with 2.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 4,5-bis[(1E)-prop-1-en-1-yl]furan-2-thiol.

### Stage 2: Synthesis of 2-sulfanyl, 3-formyl, 4,5-di(propen-1-yl), furan

1 mole of 4,5-bis[(1E)-prop-1-en-1-yl]furan-2-thiol may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2-sulfanyl, 3-formyl, 4,5-di(propen-1-yl), furan.

### Example 32

### Preparation of Compound 32-1 (2-methyl, 3-sulfanyl, 4-[(1E,3E,5E)-hepta-1,3,5-trien-1-yl], 5-formyl, furan)

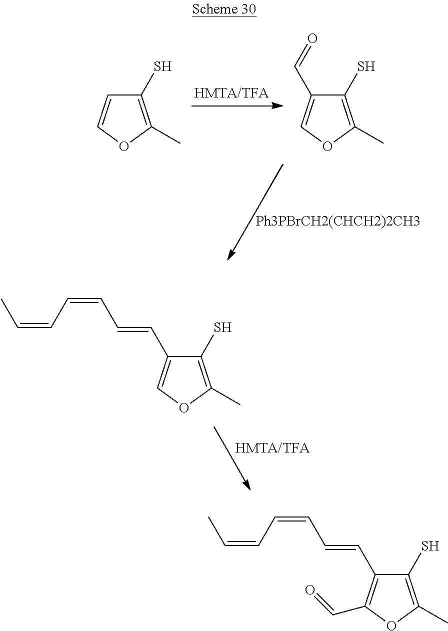

### Stage 1: Synthesis of 5-methyl-4-sulfanylfuran-3-carbaldehyde

1 mole of 2-methyl-3-sulfanylfuran may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 5-methyl-4-sulfanylfuran-3-carbaldehyde.

### Stage 2: Synthesis of 4-[(1E,3Z,5Z)-hepta-1,3,5-trien-1-yl]-2-methylfuran-3-thiol

1.0 mole of 5-methyl-4-sulfanylfuran-3-carbaldehyde may be treated with 1.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 4-[(1E,3Z,5Z)-hepta-1,3,5-trien-1-yl]-2-methylfuran-3-thiol

### Stage 3: Synthesis of 2-methyl, 3-sulfanyl, 4-[(1E,3E,5E)-hepta-1,3,5-trien-1-yl], 5-formyl, furan

1 mole of 4-[(1E,3Z,5Z)-hepta-1,3,5-trien-1-yl]-2-methylfuran-3-thiol may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2-methyl, 3-sulfanyl, 4-[(1E,3E,5E)-hepta-1,3,5-trien-1-yl], 5-formyl, furan.

### Example 33

### Preparation of Compound 33-1 (2,3-dithiol, 4-tert-butyl, 5-methyl, furan)

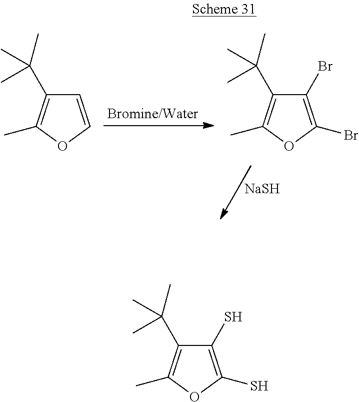

### Stage 1: Synthesis of 2,3-dibromo-4-tert-butyl-5-methylfuran

1 mole of 3-tert-butyl-3-methylfuran in water may be treated with two moles of bromine and stirred at room temperature overnight. The reaction mixture may be washed with dichloromethane and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 2,3-dibromo-4-tert-butyl-5-methylfuran.

### Stage 2: Synthesis of 2,3-dithiol, 4-tert-butyl, 5-methyl, furan

A solution of 1 mole 2,3-dibromo-4-tert-butyl-5-methylfuran may be added to a solution of 1 mole sodium hydrogen sulfide in dioxane. The reaction mixture may be stirred for 24 hours at 100° C. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2,3-dithiol, 4-tert-butyl, 5-methyl, furan.

### Example 34

### Preparation of Compound 34-1 (2,7-diacetyl, 3-hydroxy, 6-((1E,3E)-penta-1,3-dien-1-yl), benzofuran)

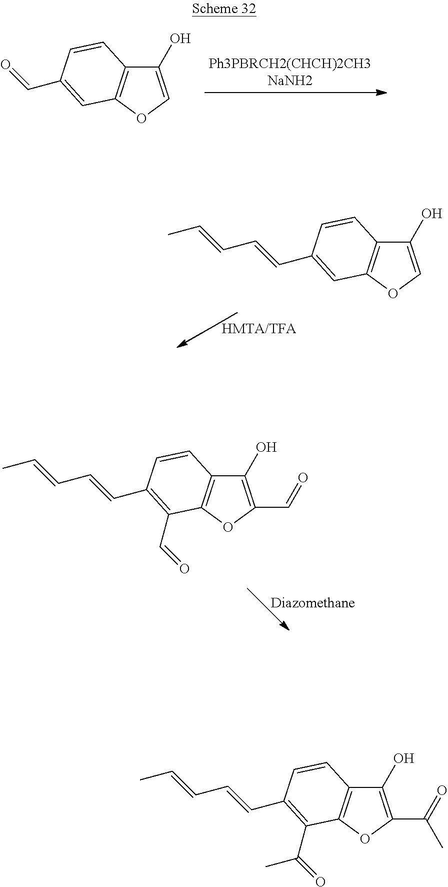 [SMILES: O=Cc1ccc2c(O)coc2c1 ; C/C=C/C=C/c1ccc2c(O)coc2c1 ; C/C=C/C=C/c1ccc2c(O)c(C=O)oc2c1C=O ; C/C=C/C=C/c1ccc2c(O)c(C(C)=O)oc2c1C(C)=O]

### Stage 1: Synthesis of 6-[(1E,3E)-penta-1,3-dien-1-yl]-1-benzofuran-3-ol

1.0 mole of 3-hydroxy-1-benzofuran-6-carbaldehyde may be treated with 1.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 6-[(1E,3E)-penta-1,3-dien-1-yl]-1-benzofuran-3-ol.

### Stage 2: Synthesis of 3-hydroxy-6-[(1E,3E)-penta-1,3-dien-1-yl]-1-benzofuran-2,7-dicarbaldehyde

1 mole of 6-[(1E,3E)-penta-1,3-dien-1-yl]-1-benzofuran-3-ol may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 3-hydroxy-6-[(1E,3E)-penta-1,3-dien-1-yl]-1-benzofuran-2,7-dicarbaldehyde.

### Stage 3: Synthesis of 2,7-diacetyl, 3-hydroxy, 6-((1E,3E)-penta-1,3-dien-1-yl), benzofuran

1 mole of 3-hydroxy-6-[(1E,3E)-penta-1,3-dien-1-yl]-1-benzofuran-2,7-dicarbaldehyde may be treated with a freshly prepared solution of 1 mole of diazomethane in tetrahydrofuran at room temperature for several hours. The solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2,7-diacetyl, 3-hydroxy, 6-((1E,3E)-penta-1,3-dien-1-yl), benzofuran.

### Example 35

### Preparation of Compound 35-1 (2,5-diethenyl, 3,7-dihydroxy, 4-formyl, 6-methyl, benzofuran)

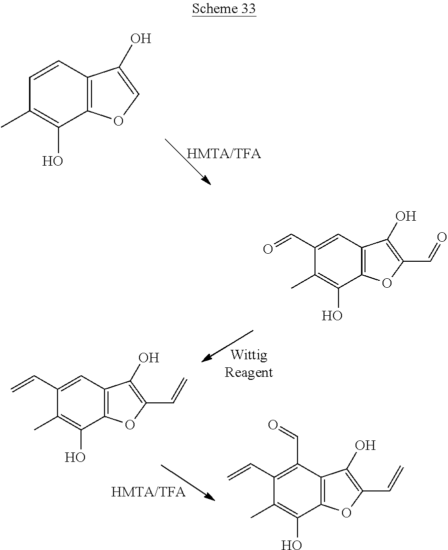 [SMILES: Cc1ccc2c(O)coc2c1O ; Cc1c(C=O)cc2c(O)c(C=O)oc2c1O ; C=Cc1cc2c(O)c(C=C)oc2c(O)c1C ; C=Cc1oc2c(O)c(C)c(C=C)c(C=O)c2c1O]

### Stage 1: Synthesis of 3,7-dihydroxy-6-methyl-1-benzofuran-2,5-dicarbaldehyde

1 mole of 6-methyl-1-benzofuran-3,7-diol may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 3,7-dihydroxy-6-methyl-1-benzofuran-2,5-dicarbaldehyde.

### Stage 2: Synthesis of 2,5-diethenyl-6-methyl-1-benzofuran-3,7-diol

1.0 mole of 3,7-dihydroxy-6-methyl-1-benzofuran-2,5-dicarbaldehyde may be treated with 2.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2,5-diethenyl-6-methyl-1-benzofuran-3,7-diol.

### Stage 3: Synthesis of 2,5-diethenyl, 3,7-dihydroxy, 4-formyl, 6-methyl, benzofuran

1 mole of 2,5-diethenyl-6-methyl-1-benzofuran-3,7-diol may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2,5-diethenyl, 3,7-dihydroxy, 4-formyl, 6-methyl, benzofuran.

### Example 36

### Preparation of Compound 36-1 (2-((1E,3E)-penta-1,3-dien-1-yl), 3-sulfanyl, 4,7-diformyl, benzofuran)

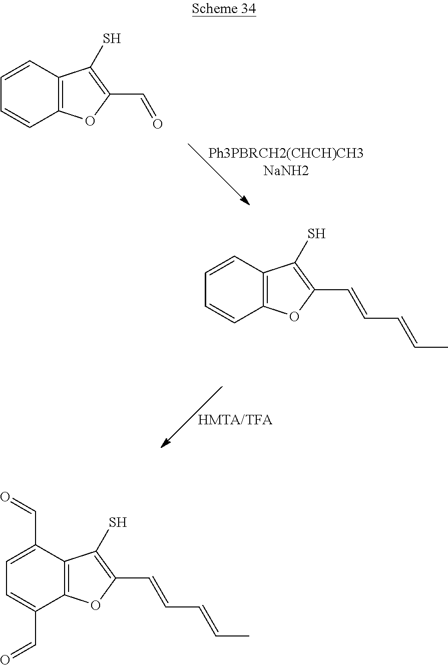 [SMILES: O=Cc1oc2ccccc2c1S ; C/C=C/C=C/c1oc2ccccc2c1S ; C/C=C/C=C/c1oc2c(C=O)ccc(C=O)c2c1S]

### Stage 1: Synthesis of 2-[(1E,3E)-penta-1,3-dien-1-yl]-1-benzofuran-3-thiol

1.0 mole of 3-sulfanyl-1-benzofuran-2-carbaldehyde may be treated with 1.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2-[(1E,3E)-penta-1,3-dien-1-yl]-1-benzofuran-3-thiol.

### Stage 2: Synthesis of 2-((1E,3E)-penta-1,3-dien-1-yl), 3-sulfanyl, 4,7-diformyl, benzofuran

1 mole of 2-[(1E,3E)-penta-1,3-dien-1-yl]-1-benzofuran-3-thiol may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2-((1E,3E)-penta-1,3-dien-1-yl), 3-sulfanyl, 4,7-diformyl, benzofuran.

### Example 37

### Preparation of Compound 37-1 (2,7-dimethyl, 4,6-diformyl, 5-hydroxy, inden-1-one)

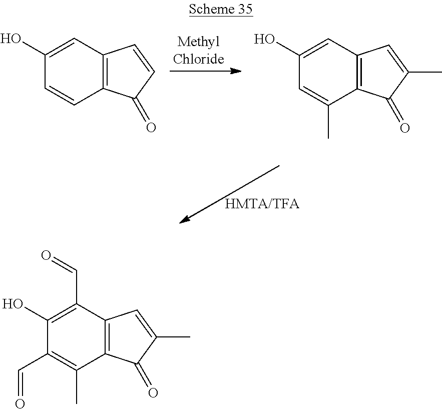 [SMILES: O=C1C=Cc2cc(O)ccc21 ; CC1=Cc2cc(O)cc(C)c2C1=O ; CC1=Cc2c(C=O)c(O)c(C=O)c(C)c2C1=O]

### Stage 1: Synthesis of 5-hydroxy-2,7-dimethyl-1H-inden-1-one

1 mole of 5-hydroxy-1H-inden-1-one may be added to a solution of 0.1 moles aluminum chloride in excess anhydrous chloromethane. The reaction mixture may be stirred at reflux overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 5-hydroxy-2,7-dimethyl-1H-inden-1-one.

### Stage 2: Synthesis of 2,7-dimethyl, 4,6-diformyl, 5-hydroxy, inden-1-one

1 mole of 5-hydroxy-2,7-dimethyl-1H-inden-1-one may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2,7-dimethyl, 4,6-diformyl, 5-hydroxy, inden-1-one.

### Example 38

### Preparation of Compound 38-1 (4-formyl, 5-hydroxy, 6-(but-2-en-1-one), dihydro-inden-1-one)

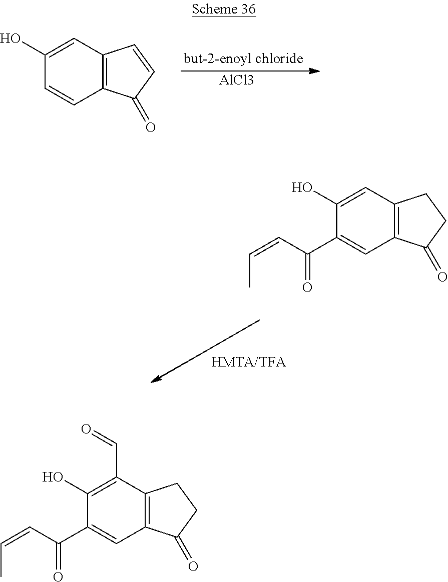 [SMILES: O=C1C=Cc2cc(O)ccc21 ; "AlCl3" ; C/C=C\C(=O)c1cc2c(cc1O)CCC2=O ; C/C=C\C(=O)c1cc2c(c(C=O)c1O)CCC2=O]

### Stage 1: Synthesis of 5-hydroxy, 6-(but-2-en-1-one), dihydro-inden-1-one

0.3 mole of aluminum chloride may be added to a solution of 1 mole freshly distilled but-2-enoyl chloride in carbon disulfide at −10° C. under nitrogen atmosphere. The mixture may be stirred for fifteen minutes and warmed to room temperature. To the reaction mixture may be added 1 mole of 5-hydroxy-dihydro-inden-1-one drop wise with cooling. The reaction mixture may be stirred at room temperature overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 5-hydroxy, 6-(but-2-en-1-one), dihydro-inden-1-one.

### Stage 2: Synthesis of 4-formyl, 5-hydroxy, 6-(but-2-en-1-one), dihydro-inden-1-one

1 mole of 5-hydroxy, 6-(but-2-en-1-one), dihydro-inden-1-one may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 4-formyl, 5-hydroxy, 6-(but-2-en-1-one), dihydro-inden-1-one.

### Example 39

### Preparation of Compound 39-1 (2-(propen-1-yl), 4-methyl, 5-sulfanyl, 6-formyl, inden-1-one)

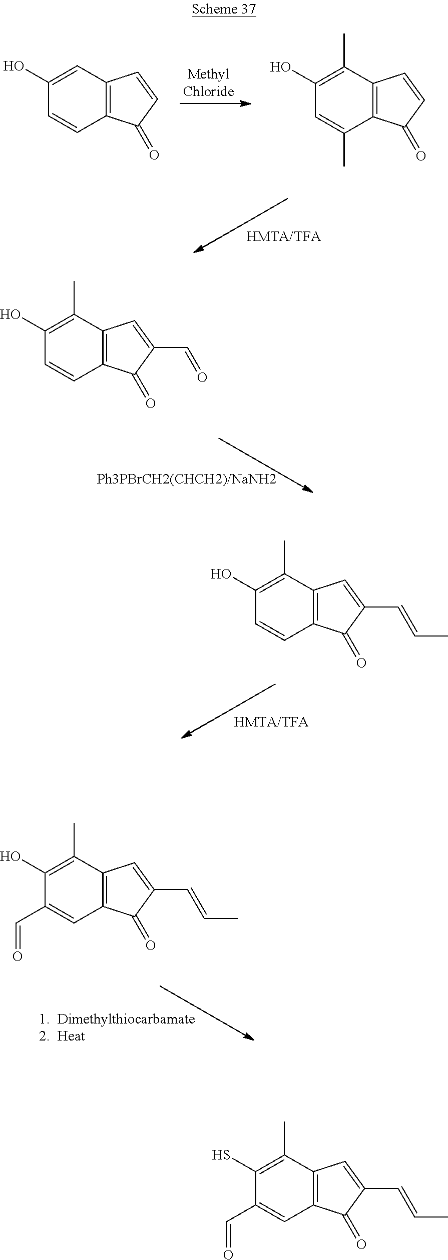 [SMILES: O=C1C=Cc2cc(O)ccc21 ; Cc1cc(O)c(C)c2c1C(=O)C=C2 ; Cc1c(O)ccc2c1C=C(C=O)C2=O ; C/C=C/C1=Cc2c(ccc(O)c2C)C1=O ; C/C=C/C1=Cc2c(cc(C=O)c(O)c2C)C1=O ; C/C=C/C1=Cc2c(cc(C=O)c(S)c2C)C1=O]

### Stage 1: Synthesis of 5-hydroxy-4-methyl-1H-inden-1-one

1 mole of 5-hydroxy-1H-inden-1-one may be added to a solution of 0.1 moles aluminum chloride in excess anhydrous chloromethane. The reaction mixture may be stirred at reflux overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 5-hydroxy-4-methyl-1H-inden-1-one.

### Stage 2: Synthesis of 5-hydroxy-4-methyl-1-oxo-1H-indene-2-carbaldehyde

1 mole of 5-hydroxy-4-methyl-1H-inden-1-one may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 5-hydroxy-4-methyl-1-oxo-1H-indene-2-carbaldehyde.

### Stage 3: Synthesis of 5-hydroxy-4-methyl-2-[(1E)-prop-1-en-1-yl]-1H-inden-1-one

1.0 mole of 5-hydroxy-4-methyl-1-oxo-1H-indene-2-carbaldehyde may be treated with 1.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 5-hydroxy-4-methyl-2-[(1E)-prop-1-en-1-yl]-1H-inden-1-one.

### Stage 4: Synthesis of 5-hydroxy-4-methyl-1-oxo-2-[(1E)-prop-1-en-1-yl]-1H-indene-6-carbaldehyde

1 mole of 5-hydroxy-4-methyl-2-[(1E)-prop-1-en-1-yl]-1H-inden-1-one may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 5-hydroxy-4-methyl-1-oxo-2-[(1E)-prop-1-en-1-yl]-1H-indene-6-carbaldehyde.

### Stage 5: Synthesis of 2-(propen-1-yl), 4-methyl, 5-sulfanyl, 6-formyl, inden-1-one

A solution of 0.5 mole triethylamine 0.5 mole of dimethylaminopyridine and 1 mole of N,N-dimethylthiocarbamoyl chloride may be added to a solution of 1 mole 5-hydroxy-4-methyl-1-oxo-2-[(1E)-prop-1-en-1-yl]-1H-indene-6-carbaldehyde in dioxane. The reaction mixture may be stirred for 24 hours at 100° C. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2-(propen-1-yl), 4-methyl, 5-sulfanyl, 6-formyl, inden-1-one.

### Example 40

### Preparation of Compound 33-1 (4,7-diethenyl, 5-sulfanyl, 6-formyl, dihydro-inden-1-one)

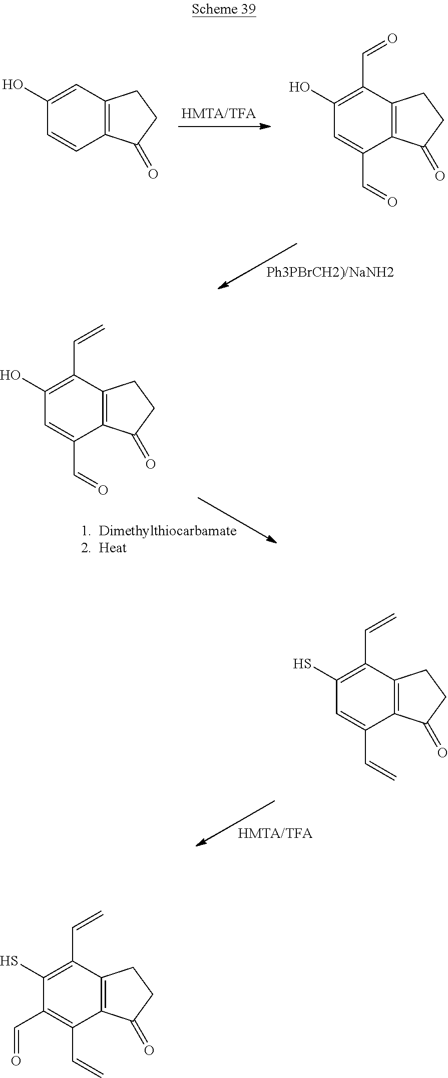 [SMILES: O=C1CCc2cc(O)ccc21 ; O=Cc1cc(O)c(C=O)c2c1C(=O)CC2 ; C=Cc1c(O)cc(C=O)c2c1CCC2=O ; C=Cc1cc(S)c(C=C)c2c1C(=O)CC2 ; C=Cc1c(S)c(C=O)c(C=C)c2c1CCC2=O]

### Stage 1: Synthesis of 5-hydroxy-1-oxo-2,3-dihydro-1H-indene-4,7-dicarbaldehyde

1 mole of 5-hydroxy-1-oxo-2,3-dihydro-1H-indene may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 5-hydroxy-1-oxo-2,3-dihydro-1H-indene-4,7-dicarbaldehyde.

### Stage 2: Synthesis of 4,7-diethenyl-5-hydroxy-2,3-dihydro-1H-inden-1-one

1.0 mole of 5-hydroxy-1-oxo-2,3-dihydro-1H-indene-4,7-dicarbaldehyde may be treated with 2.4 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 4,7-diethenyl-5-hydroxy-2,3-dihydro-1H-inden-1-one.

### Stage 3: Synthesis of 4,7-diethenyl-5-sulfanyl-2,3-dihydro-1H-inden-1-one

A solution of 0.5 mole triethylamine 0.5 mole of dimethylaminopyridine and 1 mole of N,N-dimethylthiocarbamoyl chloride may be added to a solution of 1 mole 4,7-diethenyl-5-hydroxy-2,3-dihydro-1H-inden-1-one in dioxane. The reaction mixture may be stirred for 24 hours at 100° C. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 4,7-diethenyl-5-sulfanyl-2,3-dihydro-1H-inden-1-one.

### Stage 4: Synthesis of 4,7-diethenyl, 5-sulfanyl, 6-formyl, dihydro-inden-1-one

1 mole of 4,7-diethenyl-5-sulfanyl-2,3-dihydro-1H-inden-1-one may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 4,7-diethenyl, 5-sulfanyl, 6-formyl, dihydro-inden-1-one.

### Example 41

### Preparation of Compound 41-1 (3,6-dihydroxy-9-oxofluorene-2,7-dicarbaldehyde)

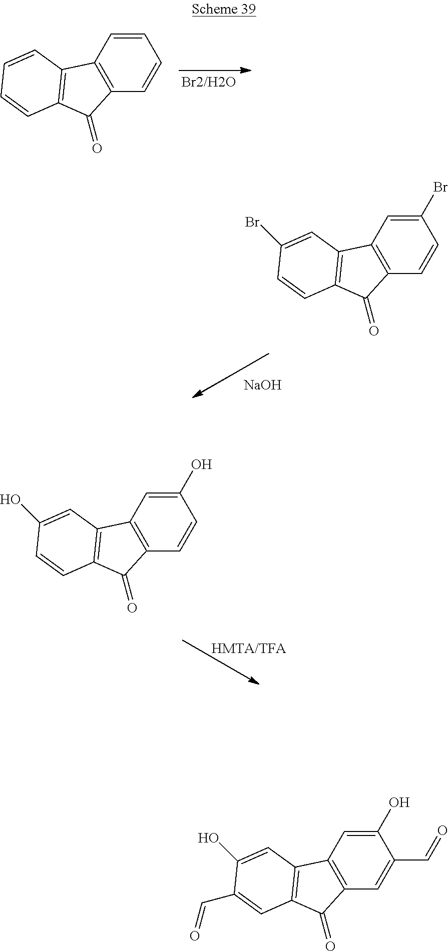 [SMILES: O=C1c2ccccc2-c2ccccc21 ; O=C1c2ccc(Br)cc2-c2cc(Br)ccc21 ; "NaOH" ; O=C1c2ccc(O)cc2-c2cc(O)ccc21 ; "HMTA/TFA" ; O=Cc1cc2c(cc1O)-c1cc(O)c(C=O)cc1C2=O]

### Stage 1: Synthesis of 3,6-dibromo-9H-fluoren-9-one

1 mole of 9H-fluoren-9-one in water may be treated with two moles of bromine and stirred at room temperature overnight. The reaction mixture may be washed with dichloromethane and the organic phase may be isolated. The organic phase may be washed twice with water and separated. The organic phase may be dried with magnesium sulfate, filtered and the solvent may be evaporated with a thin-film evaporator at elevated temperature and under reduced pressure to 3,6-dibromo-9H-fluoren-9-one.

### Stage 2: Synthesis of 3,6-dihydroxy-9H-fluoren-9-one

1 mole of 3,6-dibromo-9H-fluoren-9-one in water may be treated with two moles of sodium hydroxide and stirred at room temperature overnight. The reaction mixture may be washed with dichloromethane and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 3,6-dihydroxy-9H-fluoren-9-one.

### Stage 3: Synthesis of 3,6-dihydroxy-9-oxofluorene-2,7-dicarbaldehyde

1 mole of 3,6-dihydroxy-9H-fluoren-9-one in water may be treated with two moles of sodium hydroxide and stirred at room temperature overnight. The reaction mixture may be washed with dichloromethane and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 3,6-dihydroxy-9-oxofluorene-2,7-dicarbaldehyde.

### Example 42

### Preparation of Compound 42-1 (2-tert-butyl, 4-(2-methyl-propen-1-yl), 5-hydroxy, oxazole)

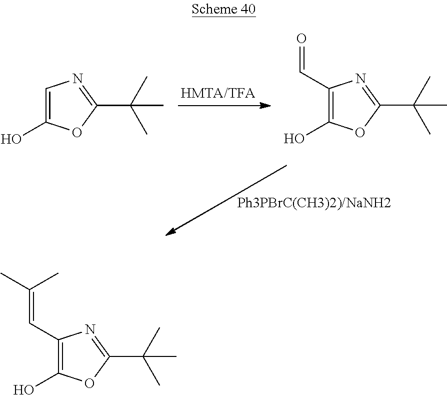

### Stage 1: Synthesis of 2-tert-butyl-5-hydroxy-1,3-oxazole-4-carbaldehyde

1 mole of 2-tert-butyl-1,3-oxazol-5-ol may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2-tert-butyl-5-hydroxy-1,3-oxazole-4-carbaldehyde.

### Stage 2: Synthesis of 2-tert-butyl, 4-(2-methyl-propen-1-yl), 5-hydroxy, oxazole)

1.0 mole of 2-tert-butyl-5-hydroxy-1,3-oxazole-4-carbaldehyde may be treated with 1.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2-tert-butyl, 4-(2-methyl-propen-1-yl), 5-hydroxy, oxazole),

### Example 43

### Preparation of Compound 43-1 (3-tert-butyl, 4-(propen-1-yl), 5-hydroxy, isoxazole)

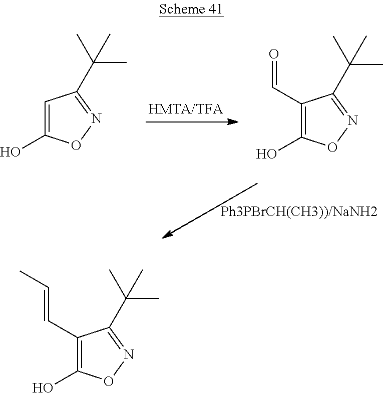

### Stage 1: Synthesis of 3-tert-butyl-5-hydroxy-1,2-oxazole-4-carbaldehyde

1 mole of 3-tert-butyl-1,2-oxazol-5-ol may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 3-tert-butyl-5-hydroxy-1,2-oxazole-4-carbaldehyde.

### Stage 2: Synthesis of 3-tert-butyl, 4-(propen-1-yl), 5-hydroxy, isoxazole

1.0 mole of 3-tert-butyl-5-hydroxy-1,2-oxazole-4-carbaldehyde may be treated with 1.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 3-tert-butyl, 4-(propen-1-yl), 5-hydroxy, isoxazole.

### Example 44

### Preparation of Compound 44-1 (4-N,4-N-dimethyl, 2,4,6-triamine, 3,5-di-(2-methylpropen-1-yl), pyridine)

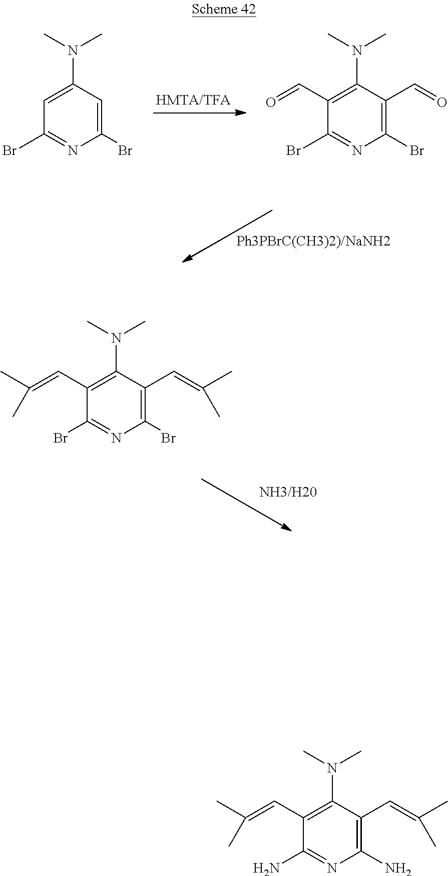 [SMILES: CN(C)c1cc(Br)nc(Br)c1 ; CN(C)c1c(C=O)c(Br)nc(Br)c1C=O ; CC(C)=Cc1c(Br)nc(Br)c(C=C(C)C)c1N(C)C ; CC(C)=Cc1c(N)nc(N)c(C=C(C)C)c1N(C)C]

### Stage 1: Synthesis of 2,6-dibromo-4-(dimethylamino)pyridine-3,5-dicarbaldehyde

1 mole of 2,6-dibromo-4-(dimethylamino)pyridine may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2,6-dibromo-4-(dimethylamino)pyridine-3,5-dicarbaldehyde.

### Stage 2: Synthesis of 2,6-dibromo-N,N-dimethyl-3,5-bis(2-methylprop-1-en-1-yl)pyridin-4-amine

1.0 mole of 2,6-dibromo-4-(dimethylamino)pyridine-3,5-dicarbaldehyde may be treated with 2.4 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2,6-dibromo-N,N-dimethyl-3,5-bis(2-methylprop-1-en-1-yl)pyridin-4-amine

### Stage 3: Synthesis of 4-N,4-N-dimethyl, 2,4,6-triamine, 3,5-di-(2-methylpropen-1-yl), pyridine

A solution of 1 mole 2,6-dibromo-N,N-dimethyl-3,5-bis(2-methylprop-1-en-1-yl)pyridin-4-amine may be added to a solution of ammonium hydroxide. The reaction mixture may be stirred for 24 hours at room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 4-N,4-N-dimethyl, 2,4,6-triamine, 3,5-di-(2-methylpropen-1-yl), pyridine.

### Example 45

### Preparation of Compound 45-1 (2,4,7-triamine, 3,5,6,8-tetraethenyl, quinoline)

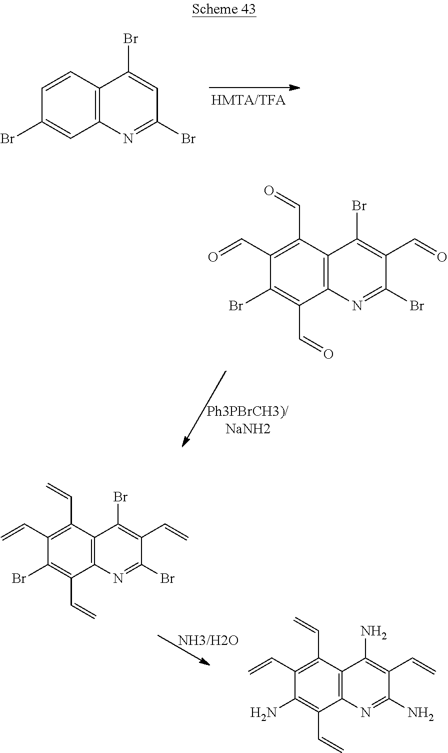 [SMILES: Brc1ccc2c(Br)cc(Br)nc2c1 ; O=Cc1c(Br)nc2c(C=O)c(Br)c(C=O)c(C=O)c2c1Br ; C=Cc1c(Br)nc2c(C=C)c(Br)c(C=C)c(C=C)c2c1Br ; C=Cc1c(N)nc2c(C=C)c(N)c(C=C)c(C=C)c2c1N]

### Stage 1: Synthesis of 2,4,7-tribromoquinoline-3,5,6,8-tetracarbaldehyde

1 mole of 2,4,7-tribromoquinoline may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2,4,7-tribromoquinoline-3,5,6,8-tetracarbaldehyde

### Stage 2: Synthesis of 2,4,7-tribromo-3,5,6,8-tetraethenylquinoline

1.0 mole of 2,4,7-tribromoquinoline-3,5,6,8-tetracarbaldehyde may be treated with 2.4 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2,4,7-tribromo-3,5,6,8-tetraethenylquinoline.

### Stage 3: Synthesis of 2,4,7-triamine, 3,5,6,8-tetraethenyl, quinoline

A solution of 1 mole 2,4,7-tribromo-3,5,6,8-tetraethenylquinoline may be added to a solution of ammonium hydroxide. The reaction mixture may be stirred for 24 hours at room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2,4,7-triamine, 3,5,6,8-tetraethenyl, quinoline.

### Example 46

### Preparation of Compound 46-1 (2,5,8-triamine, 3,4,7-trimethyl, 6-((1E)-buta-1,3-dien-1-yl), isoquinoline)

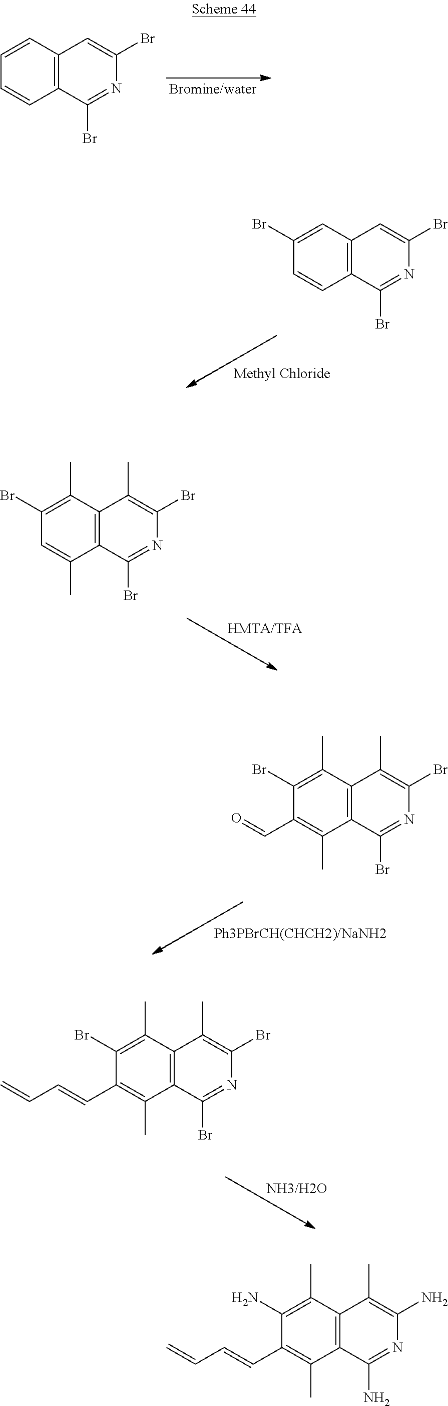 [SMILES: Brc1cc2ccccc2c(Br)n1 ; Brc1ccc2c(Br)nc(Br)cc2c1 ; Cc1cc(Br)c(C)c2c(C)c(Br)nc(Br)c12 ; Cc1c(C=O)c(Br)c(C)c2c(C)c(Br)nc(Br)c12 ; C=C/C=C/c1c(Br)c(C)c2c(C)c(Br)nc(Br)c2c1C ; C=C/C=C/c1c(N)c(C)c2c(C)c(N)nc(N)c2c1C]

### Stage 1: Synthesis of 1,3,6-tribromoisoquinoline

1 mole of 1,3-dibromoisoquinoline in water may be treated with two moles of bromine and stirred at room temperature overnight. The reaction mixture may be washed with dichloromethane and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 1,3,6-tribromoisoquinoline.

### Stage 2: Synthesis of 1,3,6-tribromo-4,5,8-trimethylisoquinoline

1 mole of 1,3,6-tribromoisoquinoline may be added to a solution of 0.1 moles aluminum chloride in excess anhydrous chloromethane. The reaction mixture may be stirred at reflux overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 1,3,6-tribromo-4,5,8-trimethylisoquinoline.

### Stage 3: Synthesis of 1,3,6-tribromo-4,5,8-trimethylisoquinoline-7-carbaldehyde

1 mole of 1,3,6-tribromo-4,5,8-trimethylisoquinoline may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 1,3,6-tribromo-4,5,8-trimethylisoquinoline-7-carbaldehyde.

### Stage 4: Synthesis of 1,3,6-tribromo-7-[(1E)-buta-1,3-dien-1-yl]-4,5,8-trimethylisoquinoline

1.0 mole of 1,3,6-tribromo-4,5,8-trimethylisoquinoline-7-carbaldehyde may be treated with 1.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 1,3,6-tribromo-7-[(1E)-buta-1,3-dien-1-yl]-4,5,8-trimethylisoquinoline.

### Stage 5: Synthesis of 2,5,8-triamine, 3,4,7-trimethyl, 6-((1E)-buta-1,3-dien-1-yl), isoquinoline

A solution of 1 mole 1,3,6-tribromo-7-[(1E)-buta-1,3-dien-1-yl]-4,5,8-trimethylisoquinoline may be added to a solution of ammonium hydroxide. The reaction mixture may be stirred for 24 hours at room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2,5,8-triamine, 3,4,7-trimethyl, 6-((1E)-buta-1,3-dien-1-yl), isoquinoline.

### Example 47

### Preparation of Compound 47-1 (4,9-diethenyl-5,10-dimethylpyrido[3,4-g]isoquinoline-1,3,6,8-tetramine)

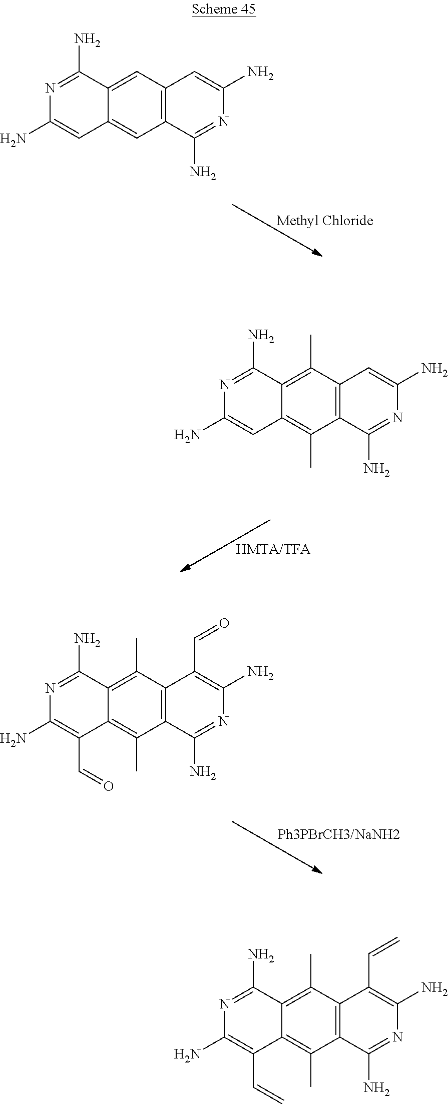 [SMILES: Nc1cc2cc3c(N)nc(N)cc3cc2c(N)n1 ; Cc1c2cc(N)nc(N)c2c(C)c2cc(N)nc(N)c12 ; Cc1c2c(N)nc(N)c(C=O)c2c(C)c2c(N)nc(N)c(C=O)c12 ; C=Cc1c(N)nc(N)c2c(C)c3c(C=C)c(N)nc(N)c3c(C)c12]

### Stage 1: Synthesis of 5,10-dimethylpyrido[3,4-g]isoquinoline-1,3,6,8-tetramine

1 mole of pyrido[3,4-g]isoquinoline-1,3,6,8-tetramine may be added to a solution of 0.1 moles aluminum chloride in excess anhydrous chloromethane. The reaction mixture may be stirred at reflux overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 5,10-dimethylpyrido[3,4-g]isoquinoline-1,3,6,8-tetramine.

### Stage 2: Synthesis of 1,3,6,8-tetramino-5,10-dimethylpyrido[3,4-g]isoquinoline-4,9-dicarbaldehyde

1 mole of 5,10-dimethylpyrido[3,4-g]isoquinoline-1,3,6,8-tetramine may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 1,3,6,8-tetramino-5,10-dimethylpyrido[3,4-g]isoquinoline-4,9-dicarbaldehyde.

### Stage 3: Synthesis of 4,9-diethenyl-5,10-dimethylpyrido[3,4-g]isoquinoline-1,3,6,8-tetramine)

1.0 mole of 1,3,6,8-tetramino-5,10-dimethylpyrido[3,4-g]isoquinoline-4,9-dicarbaldehyde may be treated with 2.4 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 4,9-diethenyl-5,10-dimethylpyrido[3,4-g]isoquinoline-1,3,6,8-tetramine).

### Example 48

### Preparation of Compound 48-1(4,6-diethenyl-5,10-dimethylpyrido[4,3-g]isoquinoline-1,3,7,9-tetramine)

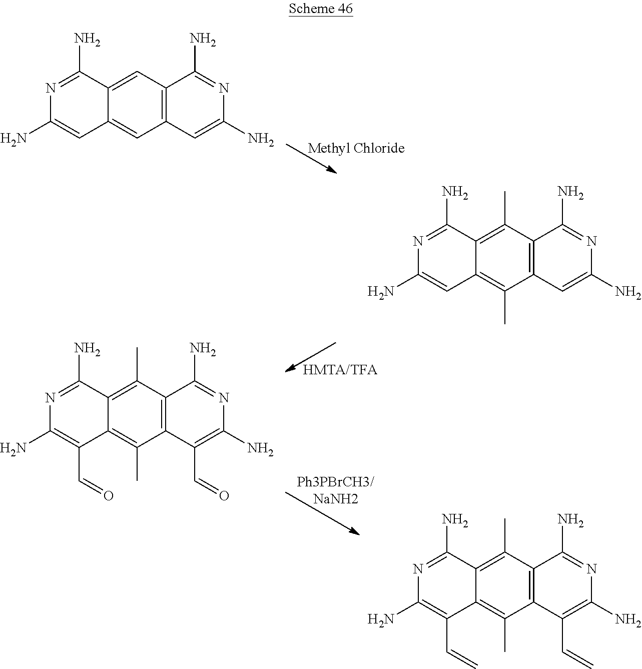 [SMILES: Nc1cc2cc3cc(N)nc(N)c3cc2c(N)n1 ; Cc1c2cc(N)nc(N)c2c(C)c2c(N)nc(N)cc12 ; Cc1c2c(N)nc(N)c(C=O)c2c(C)c2c(C=O)c(N)nc(N)c12 ; C=Cc1c(N)nc(N)c2c(C)c3c(N)nc(N)c(C=C)c3c(C)c12]

### Stage 1: Synthesis of 5,10-dimethylpyrido[4,3-g]isoquinoline-1,3,7,9-tetramine

1 mole of pyrido[4,3-g]isoquinoline-1,3,7,9-tetramine may be added to a solution of 0.1 moles aluminum chloride in excess anhydrous chloromethane. The reaction mixture may be stirred at reflux overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 5,10-dimethylpyrido[4,3-g]isoquinoline-1,3,7,9-tetramine.

### Stage 2: Synthesis of 1,3,7,9-tetramino-5,10-dimethylpyrido[4,3-g]isoquinoline-4,6-dicarbaldehyde

1 mole of 5,10-dimethylpyrido[4,3-g]isoquinoline-1,3,7,9-tetramine may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 1,3,7,9-tetramino-5,10-dimethylpyrido[4,3-g]isoquinoline-4,6-dicarbaldehyde.

### Stage 3: Synthesis of 4,6-diethenyl-5,10-dimethylpyrido[4,3-g]isoquinoline-1,3,7,9-tetramine

1.0 mole of 4,6-diethenyl-5,10-dimethylpyrido[4,3-g]isoquinoline-1,3,7,9-tetramine may be treated with 2.4 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 4,6-diethenyl-5,10-dimethylpyrido[4,3-g]isoquinoline-1,3,7,9-tetramine.

### Example 49

### Preparation of Compound 50-1 (2,4,9-triamine, 5,6,8-trimethyl, 7-[(1E)-prop-1-en-1-yl], 3H-naphtho[2,3-d]imidazole)

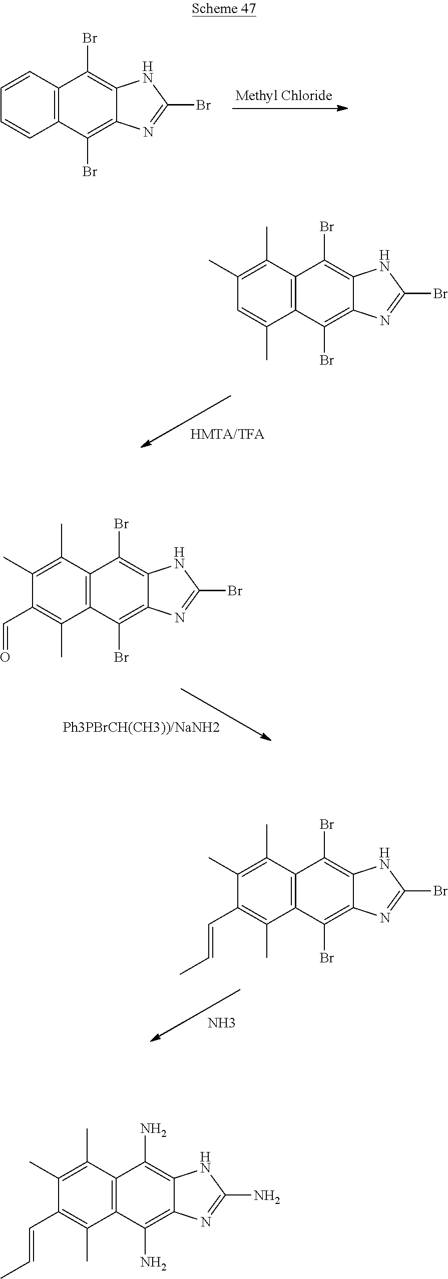

### Stage 1: Synthesis of 2,4,9-tribromo-5,7,8-trimethyl-1H-naphtho[2,3-d]imidazole

1 mole of 2,4,9-tribromo-1H-naphtho[2,3-d]imidazole may be added to a solution of 0.1 moles aluminum chloride in excess anhydrous chloromethane. The reaction mixture may be stirred at reflux overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 2,4,9-tribromo-5,7,8-trimethyl-1H-naphtho[2,3-d]imidazole.

### Stage 2: Synthesis of 2,4,9-tribromo-5,7,8-trimethyl-1H-naphtho[2,3-d]imidazole-6-carbaldehyde

1 mole of 2,4,9-tribromo-5,7,8-trimethyl-1H-naphtho[2,3-d]imidazole may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2,4,9-tribromo-5,7,8-trimethyl-1H-naphtho[2,3-d]imidazole-6-carbaldehyde.

### Stage 3: Synthesis of 2,4,9-tribromo-5,7,8-trimethyl-6-[(1E)-prop-1-en-1-yl]-1H-naphtho[2,3-d]imidazole

1.0 mole of 2,4,9-tribromo-5,7,8-trimethyl-1H-naphtho[2,3-d]imidazole-6-carbaldehyde may be treated with 1.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2,4,9-tribromo-5,7,8-trimethyl-6-[(1E)-prop-1-en-1-yl]-1H-naphtho[2,3-d]imidazole.

### Stage 4: Synthesis of 2,4,9-triamine, 5,6,8-trimethyl, 7-[(1E)-prop-1-en-1-yl], 3H-naphtho[2,3-d]imidazole

A solution of 1 mole of 2,4,9-tribromo-5,7,8-trimethyl-6-[(1E)-prop-1-en-1-yl]-1H-naphtho[2,3-d]imidazole may be added to a solution of ammonium hydroxide. The reaction mixture may be stirred for 24 hours at room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2,4,9-triamine, 5,6,8-trimethyl, 7-[(1E)-prop-1-en-1-yl], 3H-naphtho[2,3-d]imidazole.

### Example 50

### Preparation of Compound 51-1 (2,6-di-(2-methylpropen-1-yl), 3,5-diethenyl, 4-hydroxy, pyridine)

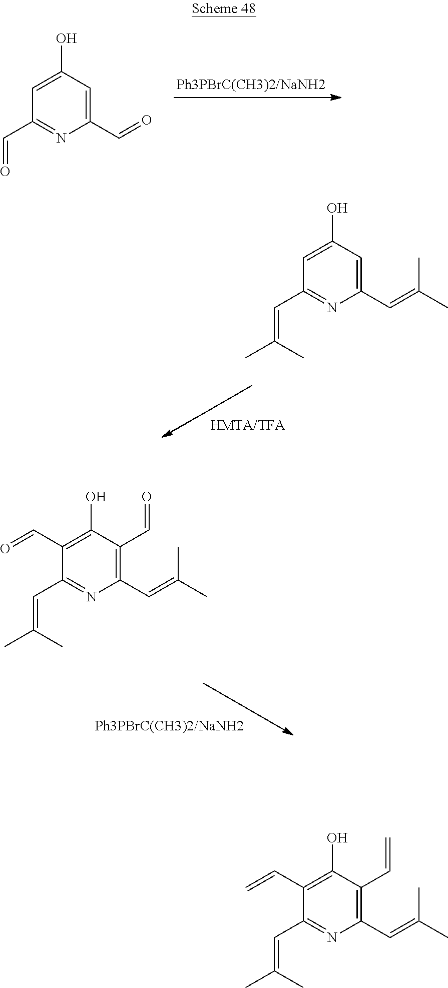 [SMILES: O=Cc1cc(O)cc(C=O)n1 ; CC(C)=Cc1cc(O)cc(C=C(C)C)n1 ; CC(C)=Cc1nc(C=C(C)C)c(C=O)c(O)c1C=O ; C=Cc1c(C=C(C)C)nc(C=C(C)C)c(C=C)c1O]

### Stage 1: Synthesis of 2,6-bis(2-methylprop-1-en-1-yl)pyridin-4-ol

1.0 mole of 4-hydroxypyridine-2,6-dicarbaldehyde may be treated with 2.4 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2,6-bis(2-methylprop-1-en-1-yl)pyridin-4-ol.

### Stage 2: Synthesis of 4-hydroxy-2,6-bis(2-methylprop-1-en-1-yl)pyridine-3,5-dicarbaldehyde

1 mole of 2,6-bis(2-methylprop-1-en-1-yl)pyridin-4-ol may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 4-hydroxy-2,6-bis(2-methylprop-1-en-1-yl)pyridine-3,5-dicarbaldehyde.

### Stage 3: Synthesis of 2,6-di-(2-methylpropen-1-yl), 3,5-diethenyl, 4-hydroxy, pyridine)

1.0 mole of 4-hydroxy-2,6-bis(2-methylprop-1-en-1-yl)pyridine-3,5-dicarbaldehyde may be treated with 2.4 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2,6-di-(2-methylpropen-1-yl), 3,5-diethenyl, 4-hydroxy, pyridine).

### Example 51

### Preparation of Compound 52-1 (5,7-diethenyl-6-methyl-1H-naphtho[2,3-b]pyrrole-9-carbaldehyde)

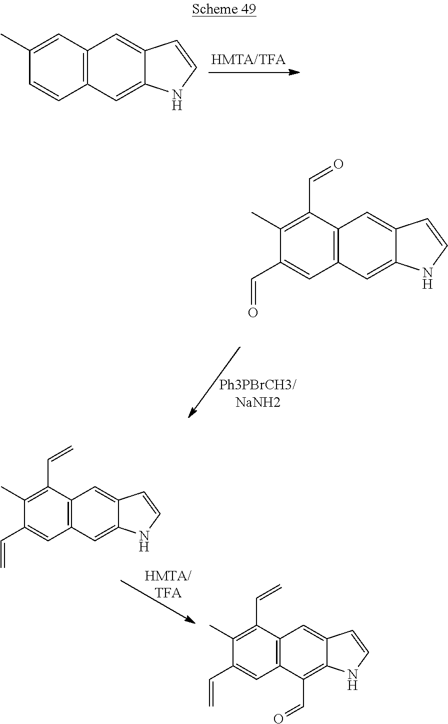 [SMILES: Cc1ccc2cc3[nH]ccc3cc2c1 ; Cc1c(C=O)cc2cc3[nH]ccc3cc2c1C=O ; C=Cc1cc2cc3[nH]ccc3cc2c(C=C)c1C ; C=Cc1cc2c(C=O)c3[nH]ccc3cc2c(C=C)c1C]

### Stage 1: Synthesis of 6-methyl-1H-benzo[f]indole-5,7-dicarbaldehyde

1 mole of 6-methyl-1H-benzo[f]indole may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 6-methyl-1H-benzo[f]indole-5,7-dicarbaldehyde.

### Stage 2: Synthesis of 5,7-diethenyl-6-methyl-1H-benzo[f]indole

1.0 mole of 6-methyl-1H-benzo[f]indole-5,7-dicarbaldehyde may be treated with 2.4 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 5,7-diethenyl-6-methyl-1H-benzo[f]indole.

### Stage 3: Synthesis of 5,7-diethenyl-6-methyl-1H-naphtho[2,3-b]pyrrole-9-carbaldehyde

1 mole of 5,7-diethenyl-6-methyl-1H-benzo[f]indole may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 5,7-diethenyl-6-methyl-1H-benzo[f]indole.

### Example 52

### Preparation of Compound 53-1 (2,3,4,5,6-pentaethenyl, pyridine)

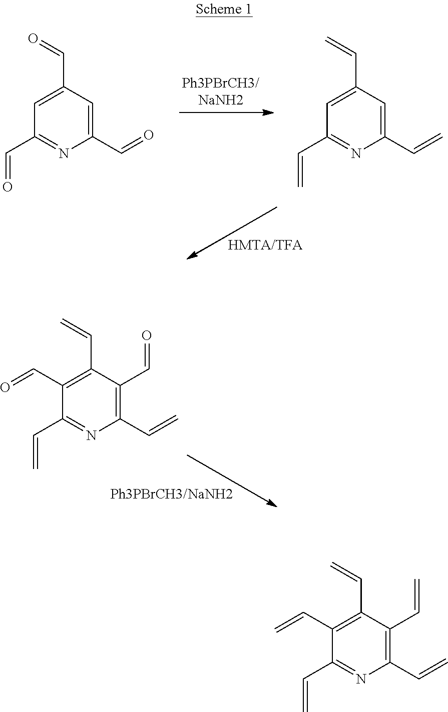 [SMILES: O=Cc1cc(C=O)nc(C=O)c1 ; C=Cc1cc(C=C)nc(C=C)c1 ; C=Cc1nc(C=C)c(C=O)c(C=C)c1C=O ; C=Cc1nc(C=C)c(C=C)c(C=C)c1C=C]

### Stage 1: Synthesis of 2,4,6-triethenylpyridine

1.0 mole of pyridine-2,4,6-tricarbaldehyde may be treated with 2.4 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2,4,6-triethenylpyridine.

### Stage 2: Synthesis of 2,4,6-triethenylpyridine-3,5-dicarbaldehyde

1 mole of 2,4,6-triethenylpyridine may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2,4,6-triethenylpyridine-3,5-dicarbaldehyde.

### Stage 3: Synthesis of 2,3,4,5,6-pentaethenyl, pyridine

1.0 mole of 2,4,6-triethenylpyridine-3,5-dicarbaldehyde may be treated with 2.4 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 2,3,4,5,6-pentaethenyl, pyridine.

### Example 53

### Preparation of Compound 54-1 (3-formyl, 5,6,8-trimethyl, 7-(propen-1-yl), quinoline)

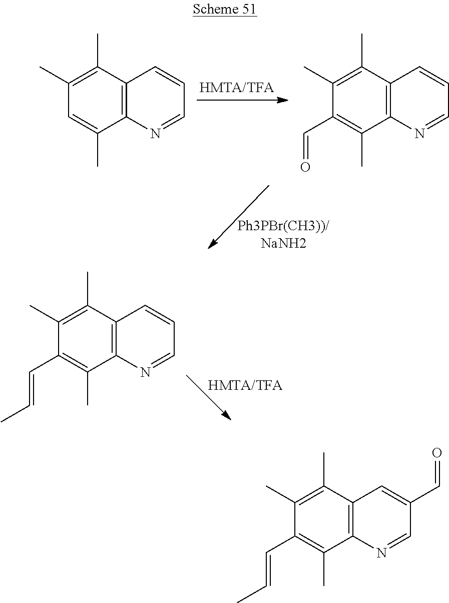 [SMILES: Cc1cc(C)c2ncccc2c1C ; Cc1c(C=O)c(C)c2ncccc2c1C ; C/C=C/c1c(C)c(C)c2cccnc2c1C ; C/C=C/c1c(C)c(C)c2cc(C=O)cnc2c1C]

### Stage 1: Synthesis of 5,6,8-trimethylquinoline-7-carbaldehyde

1 mole of 5,6,8-trimethylquinoline may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 5,6,8-trimethylquinoline-7-carbaldehyde.

### Stage 2: Synthesis of 5,6,8-trimethyl-7-[(1E)-prop-1-en-1-yl]quinoline

1.0 mole of 5,6,8-trimethylquinoline-7-carbaldehyde may be treated with 1.2 moles of a freshly prepared Wittig reagent (prepared by the reaction triphenyl phosphine with the appropriate halide followed by treatment with butyl lithium) in toluene at reflux for two hours. The reaction mixture may be cooled to room temperature, the solvent may be removed with a thin-film evaporator at elevated temperature and under reduced pressure, and the residue dissolved in ethyl acetate. The resulting organic solution may be washed twice with water and separated. The organic phase may be dried with magnesium sulfate and the solvent may be evaporated with a thin-film evaporator at elevated temperature and under reduced pressure to afford 5,6,8-trimethyl-7-[(1E)-prop-1-en-1-yl]quinoline.

### Stage 3: Synthesis of 3-formyl, 5,6,8-trimethyl, 7-(propen-1-yl), quinoline

1 mole of 5,6,8-trimethyl-7-[(1E)-prop-1-en-1-yl]quinoline may be treated with 1 mole of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 3-formyl, 5,6,8-trimethyl, 7-(propen-1-yl), quinoline.

### Example 54

### Preparation of Compound 55-1 (2,8-diformyl, 3,4,5,6,7-pentamethyl, isoquinoline)

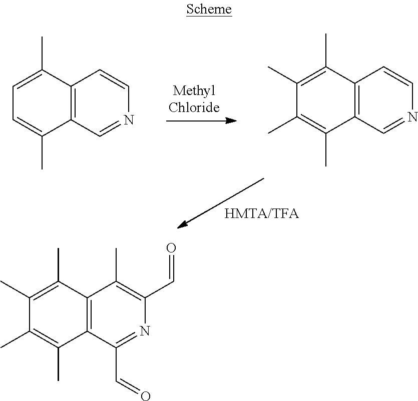 [SMILES: Cc1ccc(C)c2cnccc12 ; Cc1c(C)c(C)c2cnccc2c1C ; Cc1c(C)c(C)c2c(C)c(C=O)nc(C=O)c2c1C]

### Stage 1: Synthesis of 5,6,7,8-tetramethylisoquinoline

1 mole of 5,8-dimethylisoquinoline may be added to a solution of 0.1 moles aluminum chloride in excess anhydrous chloromethane. The reaction mixture may be stirred at reflux overnight. The reaction mixture may be added water and the organic phase may be isolated. The organic phase may be washed twice with water and separated. The organic phase may be dried with magnesium sulfate, filtered and the solvent may be evaporated with a thin-film evaporator at elevated temperature and under reduced pressure to afford 5,6,7,8-tetramethylisoquinoline.

### Stage 2: Synthesis of 2,8-diformyl, 3,4,5,6,7-pentamethyl, isoquinoline

1 mole of 5,6,7,8-tetramethylisoquinoline may be treated with 2 moles of hexamethylenetetramine in trifluoroacetic acid at reflux for three hours. The solvent may be removed with a thin-film evaporator at elevated temperature and under reduced pressure. The residue may be dissolved in 1M hydrochloric acid, extracted with dichloromethane, and the organic phase may be isolated. The organic phase may be washed with brine and the solvent may be removed with a thin-film evaporator at elevated temperature and under reduced pressure to afford 2,8-diformyl, 3,4,5,6,7-pentamethyl, isoquinoline.

### Measurement of Activity of Compounds

Various measurement procedures may be found in, for example, Martineau, Biochimica et Biophysica Acta, 1820: 133-150; 2012, Martineau, et al., Journal of Ethnopharmacology; 127(2): 396-406; 2010, Martineau, et al., Diabetes, Obesity and Metabolism; 12(2): 148-157; 2010, Eid, et al., Biochemical Pharmacology; 79(3): 444-454; 2010.

The most direct way of assessing uncoupling of oxidative phosphorylation (i.e., protonophoric activity) by a xenobiotic compound may be to measure an increase in basal oxygen consumption in isolated mitochondria, where basal may be defined as in the absence of ADP for synthesis of ATP (i.e., state 2 or 4 respiration). An increase in basal oxygen consumption in the absence of the synthesis of ATP indicates that the electron transport chain may be uncoupled from ATP synthase, or, in other words, that oxidation may be uncoupled from phosphorylation. This may be typically performed in liver mitochondria, but can also be performed in mitochondria from other tissues, such as heart or skeletal muscle. Rat tissues are most often used for the isolation of mitochondria. Oxygen consumption may be measured as a decrease in oxygen concentration in a gas-tight chamber with a Clark-type oxygen electrode in a technique known as oxygraphy.

The mitochondria isolation procedure described in Martineau, Biochimica et Biophysica Acta, 1820: 133-150; 2012 may be used. For example, mitochondria may be isolated from the liver of male Wistar rats weighing 200-225 grams. Rats may be anesthetized with sodium pentobarbital (50 mg/kg body weight). The portal vein may be cannulated and the hepatic artery and infrahepatic inferior vena cava may be ligated. The liver may be flushed with 100 ml of Krebs-Henseleit buffer (25 mM NaHCO3, 1.2 mM KH2PO4, pH 7.4, 250 mM NaCl, 4.8 mM KCl, 2.1 mM CaCl2, 1.2 mM MgSO4) at 22° C. and excised. Mitochondria may be isolated from 2 grams of tissue as per Johnson and Lardy (1967) D. Johnson, H. A. Lardy, Isolation of liver or kidney mitochondria, in: R. W. Eastbrook, M. E. Pullman (Eds.), Methods in Enzymology, Vol. 10, pp. 94-96, Academic Press, New York, N.Y., 1967. Briefly, tissue may be homogenized on ice using a Teflon potter homogenizer in ice-cold Tris-sucrose buffer (10 mM Tris, pH 7.2, 250 mM sucrose, 1 mM EGTA) and centrifuged at 600×g for 10 minutes at 4° C. The supernatant may be centrifuged at 15 000×g for 5 minutes at 4° C. The pellet may be washed once in the same buffer, centrifuged at 15 000×g, once in EGTA-free Tris-sucrose buffer, and centrifuged again. The final pellet, containing viable mitochondria, may be suspended in EGTA-free Tris-sucrose buffer and kept on ice. Protein content of the homogenate may be determined by Lowry protein assay.

The effects of compounds described herein on rate of oxygen consumption of isolated mitochondria may be assessed with a Clark-type oxygen microelectrode system with a 1 ml reaction chamber such as an Oxygraph apparatus (Hansatech Instruments; Norfolk, UK). One mg of mitochondrial protein may be added to respiration buffer (5 mM KH2PO4, pH 7.2, 250 mM sucrose, 5 mM MgCl2, 1 mM EGTA, and 2 μM of the complex I inhibitor rotenone) at 25° C. in the reaction chamber, for a final volume of 990 μl. State 4 respiration may be initiated 1 minute later by the injection of 6 mM (final concentration) of the complex II substrate succinate, and the basal rate of oxygen consumption per mg mitochondrial protein may be determined over the next 2 minutes. The test compound may then be injected and its effect on the rate of basal oxygen consumption may be assessed over 1 minute or more. Oxidative phosphorylation (state 3 respiration) may be then induced by the addition of 200 μM (final concentration) ADP and the ADP-stimulated rate of oxygen consumption per mg mitochondrial protein in the presence of the compound may be determined. Multiple runs of the vehicle-(DMSO) control may be conducted at the beginning and end of each experimental session in order to establish session-normal basal and ADP-stimulated rates of oxygen consumption, and to ensure no loss in mitochondrial viability over the duration of the session, typically less than 4 hours from the end of the isolation protocol. Such mitochondrial preparations may consistently yield a coupling ratio (ADP-stimulated rate of oxygen consumption over basal rate of oxygen consumption) of 4.5 to 5. Compounds may be all screened at 1-100 μM in 0.1% DMSO in two to three or more different mitochondrial preparations. The effect of each compound may be evaluated as: 1) the magnitude of increase in basal rate of oxygen consumption per mg protein, a direct measure of the magnitude of uncoupling effect; 2) the magnitude of decrease in functional capacity per mg protein, a measure of the magnitude of the uncoupling effect plus any concomitant inhibitory effect, where functional capacity may be defined as the difference of the ADP-stimulated rate of oxygen consumption (which may be considered the maximal functional rate of oxygen consumption) and of the basal rate of oxygen consumption (which may be considered the rate of oxygen consumption driven by proton leak and that does not contribute to ATP resynthesis). This assumes that the rate of proton leak is independent of flux through oxidative phosphorylation. Calculations may be as follows: the average functional capacity of the vehicle control experiments for a given session may be calculated by subtracting the session-average basal oxygen consumption from the session-average ADP-stimulated oxygen consumption. For 1) above, the absolute increase in basal oxygen consumption measured in a given experiment may be expressed as a percentage of the session-average vehicle control functional capacity. By this definition, complete uncoupling (≧100%) may be said to have occurred if basal oxygen consumption equals or surpasses ADP-stimulated oxygen consumption, effectively abolishing capacity for ATP synthesis. For 2) above, the functional capacity measured in a given experiment may be expressed as a percentage of the session-average vehicle control functional capacity to give the residual functional capacity. Finally, the contribution of inhibitory activity, if any, to diminished functional capacity may be estimated by subtracting the decrease in functional capacity attributable to uncoupling from the total decrease in functional capacity. In addition, dose-escalation experiments may be performed with in order to determine the concentration at which 50% uncoupling is induced (U₅₀) and to assess the concentration-activity relationship; test compound may be injected repeatedly over the course of a single experiment and the cumulative effect on basal rate of oxygen consumption may be assessed after each injection. DMSO may be confirmed to have no effect on basal oxygen consumption at a concentration of up to 2% under this paradigm.

Oxygen consumption alternatively may be measured with an XF Analyzer (Seahorse Bioscience, Inc.; Billerica, Mass.) in which multiple reactions (24 or 96) may be monitored simultaneously in real time and liquid handling (i.e., Injections into the gas-tight chambers) may be automated. The apparatus can measure oxygen consumption in isolated mitochondria, as well as in cultured cells or in tissues ex-vivo, and may be suitable for use with bacterial cultures.

Protocol for ³H-deoxyglucose uptake assay in C2C12 or other skeletal muscle cells; C2C12 murine skeletal myoblasts (American Type Culture Collection; Manassas, Va.) may be cultured under standard conditions in 12-well plates. Cells may be proliferated to 80% confluence in high-glucose Dulbecco's modified Eagle's medium (DMEM) supplemented with 10% fetal bovine serum (FBS), 10% horse serum (HS), and antibiotics. Differentiation into multinucleated myotubes may be then promoted with DMEM supplemented with 2% HS and antibiotics. All uptake assays may be performed on 7-day differentiated cells, and treatment onset may be timed accordingly. Cells may be treated with reference substances (such as metformin (100 or 400 μM), phenformin (100 μM), or 2,4-dinitrophenol 10-100 μM), compounds described herein, or vehicle (DMSO) in complete differentiation medium. DMSO concentration may be fixed at 0.1% for all conditions. Cells may be routinely inspected for abnormal morphology by phase-contrast microscopy at the conclusion of the treatment period. Thirty minutes prior to uptake experiments, cells may be equilibrated in Krebs-phosphate buffer (KPB; 20 mM HEPES, 4.05 mM Na2HPO4, 0.95 mM NaH2PO4, pH 7.4, 120 mM NaCl, 5 mM glucose, 4.7 mM KCl, 1 mM CaCl2 and 1 mM MgSO4) at 37° C. Insulin, prepared freshly, may be added to some vehicle control wells at 100 nM during this period. Cells may be then washed twice in glucose-free KPB at 37° C. before incubation for exactly 10 min at 37° C. in glucose-free KPB containing 0.5 μCi/ml 2-deoxy-D-[1-3H]glucose (Amersham Biosciences; Buckinghamshire, UK). Cells may be then placed on ice and immediately washed three times with ice-cold KPB. Cells may be inspected for monolayer detachment and lysed in 0.1 N NaOH with scraping. Lysates may be transferred to scintillation fluid (e.g., Ready-Gel; Beckman Coulter Inc.; Fullerton, Calif.) and incorporated radioactivity may be assessed in a liquid scintillation counter (e.g., 1219 RackBeta; Perkin-Elmer, Waltham, Mass.). Three or more independent experiments of 18 hours treatment duration may be performed for each test compound, with three or more replicates per condition per experiment. Vehicle-control and 2,4-dinitrophenol conditions may be included on every plate for the purpose of standardizing.

Protocol for assay of glucose-6-phosphatase activity in H4IIE or other hepatocytes: H4IIE murine hepatocytes (American Type Culture Collection) may be cultured to confluence in 12-well plates in Dulbecco's modified Eagle's medium (DMEM) supplemented with 10% fetal bovine serum (FBS), and antibiotics. Cells may be treated with insulin (100 nM; prepared freshly), metformin (100-400 μM), or 2,4-dinitrophenol (10-100 μM) as positive controls or reference substances, or other with compounds described herein, or vehicle (DMSO) for 16 hours in serum-free medium. Effects of test compounds on cellular viability may be assessed by measuring the release of lactate dehydrogenase (LDH) into the culture medium at the end of a 16 hour treatment using a commercial kit (e.g., Cytotoxicity Detection Kit; Roche Diagnostics; Laval, QC) as per the manufacturer's instructions; LDH release may be expressed as % of total (i.e., medium+lysate) LDH content for each well. Cells may also be routinely inspected for abnormal morphology by phase-contrast microscopy at the conclusion of the treatment period. Following treatment, cells may be washed in HEPES-buffered saline (10 mM HEPES, pH 7.4, 150 mM NaCl) at 37° C. Glucose-6-phosphatase (G6Pase) activity may be assessed by measuring the rate of glucose formation in the presence of a non-limiting amount of glucose-6-phosphate (G6P). Glucose production may be measured with a commercial glucose assay kit (e.g., AutoKit Glucose; Wako Diagnostics; Richmond, Va.). Two hundred μl of AutoKit Glucose buffer solution diluted 1:4 in water may be added to each well. Cells may be lysed by the addition of 50 μl of 0.05% Triton X-100 in similarly-diluted AutoKit Glucose buffer solution. Immediately following addition of Triton X-100, 20 mM (final concentration) of G6P may be added to each well for a final volume of 2754 Plates may be incubated for exactly 40 min. At 37° C., after which time 500 μl of AutoKit Glucose color reagent may be added and incubation may be continued for exactly 5 minutes. Samples may be rapidly transferred to microcentrifuge tubes. Fifty μl may be removed for assay of total protein content in order to account for effects of test compounds on cellular viability or proliferation. A commercial protein assay kit based on the Bradford method may be used (e.g., Protein Assay; Bio-Rad Laboratories; Hercules, Calif.). This assay may be observed to be unaffected by high concentrations of phenolic compounds. The remaining volume may be centrifuged at 3000×g for 5 min. Absorbance of the supernatant may be measured at 505 nm at ambient temperature and glucose concentration may be calculated from a standard curve performed in parallel. Control wells without exogenous G6P may be included on each plate for each treatment condition, and activity measured from these wells may be subtracted from activity measured in the presence of exogenous G6P. G6Pase activity calculated in this way may be expressed normalized to protein content on a well-by-well basis. Three or more independent experiments in cells of different passages may be performed for each test compound, with four to six or more replicates per condition per experiment.

In either muscle or liver cells, insulin resistance may be induced by treating cells hours to days with palmitate. For example, 16 hours or more of treatment of H4IIE hepatocytes with 0.25 mM palmitate in 2% free-fatty-acid (FFA)-free bovine serum albumin decrease by more than 50% the phosphorylation of the signaling protein AKT, a marker of the insulin-signaling pathway, in response to insulin stimulation. Chronic treatment (i.e., on the order of 1 or more days) with active compounds can restore this sensitivity (i.e., Insulin-sensitizing effect), measured again as activation of a marker of the insulin-signaling pathway in response to insulin stimulation. Typically, this is assessed as magnitude of increase in phosphorylation of AKT within five to thirty minutes of insulin stimulation, measured by western immunoblotting using a phosphospecific antibody against AKT (phosphorylated at Ser473; cat. #9271; Cell Signaling Technology, Inc.; Danvers, Mass.). The endpoint may also be a reduction in the concentration of intracellular lipids. Triglyceride content is a good marker of intracellular lipid accumulation and can be measured using a variety of enzymatic assays or fluorescent assays based on the Nile Red fluorescent dye (e.g., AdipoRed Assay Reagent; Lonza Inc.; Allendale, N.J.).

Given that the therapeutic effects of the compounds are mediated by the AMP-activated protein kinase (AMPK) signaling pathway, assessing activation of this pathway may be used as an endpoint of activity for compounds described herein. This is typically achieved by measuring the level of phosphorylation of AMPK or of downstream signaling proteins (e.g., acetyl-CoA carboxylase or ACC) by western immunoblotting or ELISA techniques, using phospho-specific antibodies (phosphorylation of the alpha subunit of AMPK at Thr 172; cat. #2531; Cell Signaling Technology, Inc.; Danvers, Mass.) (phosphoryaltion of ACC at Ser79; cat. #3661; Cell Signaling Technology, Inc.; Danvers, Mass.). In-gel kinase assays can also be performed. Activation of the AMPK pathway may be assessed in muscle or liver cells within minutes to hours of treatment with test compounds.

Protein immunoblotting protocol for phosphorylated AMPK or phosphorylated ACC or phosphorylated AKT in muscle cells or hepatocytes: Contents of phosphorylated AMPKalpha (catalytic subunit; Thr 172) and phosphorylated acetyl-coA carboxylase (ACC; Ser 79) may be measured as markers of activation of the AMPK pathway. Content of phosphorylated AKT (Ser473) may be measured as a marker of the activation of the insulin receptor pathway. Reagents may be purchased from Sigma-Aldrich unless otherwise noted. Primary antibodies may be purchased from Cell Signaling Technologies, Inc. (cat. #2531, 3661, and 9271, respectively; Danvers, Mass.). Horseradish peroxidase-conjugated anti-rabbit IgG secondary antibody may be purchased from Jackson ImmunoResearch Laboratories, Inc. (cat. #111-035-144; West Grove, Pa.). C2C12 or other skeletal muscle cells may be seeded in 6-well plates and proliferated and differentiated as above. H4IIE or other hepatocytes may be cultued as above. For muscle cells, cells may be treated with test compound or vehicle (DMSO) 18, 6, or 1 h before lysis on day 7 of differentiation. 5-Aminoimidazole-4-carboxamide-1-riboside (AICAR; Toronto Research Chemicals, Inc.; North York, ON) may be used as a positive control for activation of the AMPK pathway; AICAR may be dissolved in water and applied at a final concentration of 4 mM to a subgroup of vehicle-control wells 30 minutes prior to lysis. 2,4-dinitrophenol may also be used as positive control for activation of the AMPK pathway. Insulin (100 nM; prepared freshly) applied for 30 minutes may be used as positive control for the insulin-signaling pathway. At the end of the treatment period, plates may be placed on ice and cells may be rinsed twice with ice-cold phosphate-buffered saline (PBS; 8.1 mM Na2HPO4, 1.47 mM KH2PO4, pH 7.4, 2.68 mM KCl, 0.137 M NaCl) and covered with 250 μl/well of HEPES lysis buffer (50 mM HEPES, pH 7.4, 150 mM NaCl, 5 mM EGTA, 2 mM MgCl₂, 5% glycerol, 1% Triton X-100, 1% sodium deoxycholate, 0.1% sodium dodecyl sulphate (SDS)) containing a cocktail of protease inhibitors (e.g., Complete-Mini EDTA-free; Roche Diagnostics; Laval, QC; supplemented with 1 mM phenylmethylsulphonyl fluoride) and phosphatase inhibitors (10 mM sodium fluoride, 100 μM sodium orthovanadate, 1 mM sodium pyrophosphate). Cells may be scraped and transferred to microcentrifuge tubes. The tubes may be vortexed and kept on ice for 30 minutes with frequent vortexing. Tubes may be then centrifuged at 600×g for 10 minutes at 4° C. The supernatants may be decanted into new tubes, and these lysates may be frozen at −80° C. until analysis. The protein content of the cell lysates may be measured using a bicinchoninic acid (BCA) protein assay kit (Thermo Fisher Scientific, Inc.; Waltham, Mass.), according to the manufacturer's instructions. An equal amount of total protein from each sample may be denatured by boiling 5 minutes in reducing sample buffer (62.5 mM Tris, pH 6.8, 10% glycerol, 5% β-mercaptoethanol, 1% SDS). One hundred μg of each sample in a 100 μl volume may be resolved by SDS-polyacrylamide gel electrophoresis using a Protean IIxi apparatus (Bio-Rad Laboratories; Hercules, Calif.). The resolving gel may be composed of an 8% acrylamide phase or of a 6.5% acrylamide phase over a 10% acrylamide phase, and the stacking gel may be 5% acrylamide. Electrophoresis may be performed at 4° C. in migration buffer (25 mM Tris, pH 8.3, 192 mM glycine, 0.1% SDS) at 50 mA for 3 h followed by 25 mA for 14 h. Resolved samples may be then electrotransferred to Immobilon-P polyvinylidene fluoride membrane (Millipore Corp.; Billerica, Mass.) using a Trans-Blot cell (Bio-Rad Laboratories) in transfer buffer (25 mM Tris, pH 8.3, 192 mM glycine, 10% methanol, 0.02% SDS) at 4° C., 900 mA, for 1.5 h. Membranes may be stained with Ponceau Red to confirm equal loading, then blocked for 1.5 h in 5% bovine serum albumin (BSA) dissolved in Tris-buffered saline (TBS) plus Triton X-100 (TBST; 50 mM Tris, pH 7.4, 150 mM NaCl, 0.5% Triton X-100). Blocked membranes may be incubated overnight at 4° C. with constant agitation in primary antibody solution (antibody at 1:1,000 in TBST plus 1% BSA and 0.5% sodium azide). Membranes may be rinsed in TBST and incubated 1.5 h at ambient temperature in secondary antibody solution (antibody at 1:100,000 in TBST plus 0.5% BSA). Membranes may then be thoroughly washed in TBST and TBS, and treated for 1 minute with ECL reagent (Amersham/GE Healthcare; Baie d'Urfé, QC). Membranes may be exposed to blue-light sensitive ECL film (Amersham/GE Healthcare) for the appropriate duration for maximal signal without film saturation. Films may be developed manually using D-19 developer and RapidFixer (Eastman Kodak Co.; Rochester, N.Y.). Developed films may be scanned using a Hewlett Packard 6100 flatbed scanner (HP; Palo Alto, Calif.) with HP DeskScan II software. Densitometry analysis may be then performed using Image 1.63 software (National Institutes of Health; Bethesda, Md.). Three replicates or more, each from a different cell passage, may be performed for each condition. For each series of replicates, all samples may be simultaneously subjected to electrophoresis and transferred to a single membrane. Data from the densitometric analysis of each replicate series may be normalized to the vehicle control of that series. Normalized data from the three series may be then pooled.

Stimulation of the AMPK pathway in muscle cells leads to increased capacity for glucose uptake through increased expression of glucose transporter proteins. Increases in the capacity for glucose uptake can be assessed indirectly by measuring the content of glucose transporter proteins GLUT1 and GLUT4. This can be done by western immunoblotting in muscle cells treated on the order of 1 or more days with compounds described herein. Western immunoblotting may be performed as detailed above. Anti-GLUT1 and anti-GLUT4 antibodies can be sourced from Santa Cruz Biotechnology, Inc (Santa Cruz, Calif.). Optimal antibody concentration should be determined emperically.

Compounds described herein may stimulate mitochondrial biogenesis and increase oxidative capacity through the AMPK pathway, in much the same way as endurance exercise training. These effects are relevant to restoring insulin sensitivity since they can contribute to ridding muscle and liver cells of intracellular fat, causal to insulin resistance. Increased oxidative capacity can be assessed by enzymatic assays for such key enzymes as citrate synthase following treatment of muscle or liver cells on the order of one or more days with test compounds. Citrate synthase activity may be assessed as per the method of Srere, P. A. (1969), Methods in Enzymology, XIII, 1-11.

Compounds described herein may acutely alter fuel preference and increase the oxidation of fats, again through the AMPK pathway. These effects are relevant to restoring insulin sensitivity since they can contribute to ridding muscle and liver cells of intracellular fat, causal to insulin resistance. This can be measured by incubating cells with ¹⁴C radiolabelled palmitate and then capturing expired ¹⁴C radiolabelled CO₂. Alternatively, a Seahorse Bioscience XF Analyzer can be used to measure oxygen consumption and CO₂ production, from which a change in the respiratory quotient can be calculated. Changes in fuel preference may be assessed in muscle or liver cells within minutes to hours of treatment with test compounds.

Insulin resistance is associated not only with intracellular accumulation of fats in muscle and liver cells, but also with increased oxidative stress in these cells resulting from low flux through the electron transport chain and a high mitochondrial membrane potential, conditions promoted by energy surfeit. Through their uncoupling/short-circuit effect, compounds described herein may decrease the production of oxygen free radicals by promoting flux through the electron transport chain and a decrease in mitochondrial membrane potential (i.e., relieving the pressure). It is therefore relevant to assess a decrease in oxidative stress. Muscle or liver cells can be incubated with palmitate to promote oxidative stress, then treated with test compounds on the order of hours; a variety of markers can be then measured, including mitochondrial membrane potential using fluorescent probes (e.g., JC-1 dye; Invitrogen Corp.; Grand Island, N.Y.).

Assays in Tissues Ex-Vivo or In-Situ:

Several of the endpoints listed above, namely those measured following treatment on the order of minutes to a few hours, can be assessed in isolated tissues rather than in cultured cells. For example, glucose uptake can be performed in isolated mucles ex-vivo, maintained in an oxygenated tissue bath, or in an in-situ perfusion system, such as a perfused hindlimb. These systems are considered more physiological than cell lines. See, for example, Szabo et al, Hormone and Metabolic Research; 1(4):156-61; 1969, and Gemmill, Bulletin of the Johns Hopkins Hospital; 66: 232; 1940.

Animal Models:

a) Models of chemically-induced diabetes: Destruction of insulin-producing pancreatic cells (i.e., pancreatic beta cells) can be achieved through a number of ways. The most common involves a single injection of streptozotocin (in rats, a dose of 65 mg streptozotocin/kg body weight, injected intra-peritoneally is typical). Alloxan can also be used. Within a few days, the animal (typically a rat) will exhibit very high fasting glycemia, indicating loss of glycemic control (i.e., diabetes). Compounds described herein, administered orally or by injection to a fasted rat, may cause an acute partial normalization of this hyperglycemia within minutes to hours, and potentially lasting several hours. Repeated treatments may cause a chronic effect (i.e., a cumulative effect) as changes in gene expression contribute to the glycemic control. Glycemia can be measured using a human portable blood glucose meter. The tip of the tail can be cut in order to draw a few drops of blood every 15 to 30 minutes, in order to monitor change in glycemia over time following acute administration of the test compound.

b) Models of insulin resistance: Insulin resistance (i.e., pre-diabetes) is characterized by post-prandial hyperglycemia, but normal fasting glycemia. Insulin resistance can be induced in mice and rats by promoting obesity. This can be accomplished by placing normal mice (e.g., C57BL6 mice) or normal rats (e.g., Wistar rats) on a high-fat diet (e.g., 60% of calories derived from fat; mostly lard). Such rodent chow is available from Bio-Serv (Frenchtown, N.J.); for example, product F3282/S3282 is designed for mice. Alternatively, genetically hyperphagic animal lines (i.e., animals with a genetic defect in their appetite control mechanim) can be used, such as the KK^{ay} and the db/db mouse lines; after several weeks of ad-libitum access to food, these animals become obese and insulin-resistant (typically by week 12 of life). Such animals are available from The Jackson Laboratory (Bar Harbor, Me.). Test compounds may be mixed into the animal's food in powdered form at an emperically-determined optimal concentration; a starting point for dose searching may be a 0.5% weight/weight mixture of test compound and food. As the mixture may affect taste, food intake may be decreased; in such cases, control animals may be pair-fed (i.e., allowed to eat only as much as the experimental groups have eaten on the previous day). Alternatively, test compound may be administered by intra-gastric gavage or by injection once or more times daily. Because the compounds described herein are lipophilic, they may be solubilized in a pharmaceutically-appropriate solvent (e.g., ethanol, DMSO, ethyl acetate, etc.), and then diluted in water (up to 1000× dilution, for a final solvent concentration of 0.1%). The optimal concentration of the test compound should be determined empirically.

Experiments can follow a treatment paradigm, whereby treatment is initiated once an animal has become insulin resistant, or can follow a prevention paradigm, whereby treatment is initiated at the same time as the animal is placed on the high-fat diet or given unrestricted access to food. Insulin resistance, or insulin sensitivity, is assessed by non-terminal glucose-tolerance tests: animals are administered by intra-gastric gavage a glucose solution (e.g., 2 g of glucose per kg body weight) in order to increase their glycemia; blood glucose is then sampled over a two-to-three hour period to assess the rate at which normoglycemia is restored. Only a small volume of blood is required at each time point. As above, glycemia can be measured using a human portable blood glucose meter. The tip of the tail can be cut in order to draw a few drops of blood every 15 to 30 minutes. A more sensitive assay is the hyperinsulinemic/euglycemic glucose clamp test whereby an animal is perfused with insulin and one measures how much glucose should be co-administered to maintain a normal blood glucose concentration. See, for example, Kim, Methods in Molecular Biology: Type 2 Diabetes; (560): 221-238; 2009, ISBN: 978-1-934115-15-2. For general information on the measurement of glucose homeostasis, see, for example, Ayala et al., Disease Models & Mechanisms; 3(9-10): 525-534; 2010.

The main clinical effect expected to result from an overdose of the compounds described herein is lactic acidosis. Lactatemia may be monitored during dose-searching experiments using a lactate analyzer (e.g. Lactate Plus; Nova Biomedical; Waltham, Mass.).

Once the protocol is terminated and animals are sacrificed, various measurements can be performed on their tissues post-mortem, such as concentration of muscle glucose transporters or oxidative capacity/mitochondrial density. The protein immunoblot procedure described in detail above can be adapted to animal tissues such as skeletal muscle (typically ankle extensors) and liver. The tissues are harvested under terminal anaesthesia and flash-frozen in liquid nitrogen. They are then powdered under liquid nitrogen and lysed in lysis buffer as follows:

Frozen muscles and liver samples may be powdered under liquid nitrogen. Powdered tissue (approximately 100 mg) may be solubilized by frequent vortexing over a period of 1 hour in 10 volumes of ice-cold radioimmunoprecipitation assay buffer (RIPA buffer; 50 mM HEPES, 150 mM NaCl, 5% glycerol, 5 mM EGTA, 2 mM MgCl2, pH 7.4, 1% Triton X-100, 1% sodium deoxycholate, 0.1% sodium dodecyl sulfate) containing a cocktail of protease inhibitors (e.g., Complete Mini; Roche Diagnostics; Laval, QC) and 2 mM phenylmethanesulfonyl fluoride) and phosphatase inhibitors (100 μM sodium orthovanadate, 1 mM sodium pyrophosphate, 10 mM sodium fluoride). The homogenate may be centrifuged 60 min at 4500×g, 4° C., and the supernatant may be decanted. Protein concentration of the supernatant may be determined by Bradford protein assay (Bio-Rad Canada; Mississauga, ON) in aliquots diluted 2000 times in water. Samples containing 200 μg of protein may be prepared for separation by SDS-PAGE by dilution and boiling in reducing sample buffer (60 mM Tris, 10% glycerol, 2% sodium dodecyl sulfate, 5% β-mercaptoethanol, pH 6.8). The citrate synthase activity assay detailed in Srere, P. A. (1969), Methods in Enzymology, XIII, 1-11 above can also be performed on frozen animal processed in this same way.

Due to their stimulatory effects on the AMPK pathway, the compounds described herein may also be useful for the treatment of cancer; induction of a metabolic stress and the resulting activation of the AMPK pathway leads to inhibition of non-essential energy-consuming processes including protein synthesis and cell divison, effects that may slow the growth of tumorous cells. Anti-cancer effects may be assessed in-vitro using a variety of tumorous cell lines. An endpoint may be the rate of cell proliferation, as read by the rate of incorporation of ³H radio-labelled thymidine or nucleoside analogs of thymidine into DNA as cells divide, following a treatment with test compound on the order of several hours. Effects may be assessed in-vivo using models of chemically-induced cancer. For example, breast cancer can be induced by injection of alkylating agents N-methyl-N-nitrosourea or ethylnitrosourea. Endpoints may be tumor mass and tumor number following treatment over several weeks, whereby the test compound is administered in the animal's food, or by intragastric gavage, or by injection, as described above.

The compounds described herein may kill or cause a bacteriostatic effect in aerobic bacteria. These effects may be measured in cultured bacteria in-vitro. See, for example, Cappuccino et al, Microbiology: A Laboratory Manual, 9th Edition (2010). Such effects may be therapeutically relevant, for example, for the prevention of oral carries or for the treatment of stomach ulcers caused by H. pylori. They may be useful externally as topical agents. In cases of internal use, compounds such as those described herein may be designed so as to combine anti-bacterial activity with low bioavailability to humans or absence of activity in mitochondria, either strategy potentially increasing therapeutic safety.

The compounds described herein may be used as anti-fungals for the treatment of wood or leather or other substances. Anti-fungal activity can be measured in cultured fungi in-vitro. See, for example, Koneman, et al. Practical Laboratory Mycology; 3rd edition (1985) Williams and Wilkins (Baltimore, Md.). As in bacteria, both death and inhibition of growth may be of interest.

The compounds described herein may be toxic to some plants and may therefore be used as herbicides. Such activity may be measured in cultured plants. See, for example, Naylor, ed. Weed Management Handbook, 9th Edition (2002), Wiley-Blackwell and Monaco et al, Weed Science: Principles and Practices, 4th Edition (2002). The compounds described herein may be expected to exhibit low environmental persistence, distinguishing them from existing uncoupler-based herbicides. For improved safety, compounds such as those described herein may be designed for activity in chloroplasts and absence of activity in mitochondria.

The compounds described herein may be toxic to some insects and other pests and may therefore be used as pesticides. Such activity can be measured in cultures. See, for example, Bohnmont, The Standard Pesticide User's Guide, 7th edition (2006). The compounds described herein may be expected to exhibit low environmental persistence, distinguishing them from existing uncoupler-based pesticides. For improved safety, compounds such as those described herein may be designed for low gastro-intestinal bioavailabilty in higher animals.

In the claims provided herein, the steps specified to be taken in a claimed method or process may be carried out in any order without departing from the principles of the invention, except when a temporal or operational sequence is explicitly defined by claim language. Recitation in a claim to the effect that first a step is performed then several other steps are performed shall be taken to mean that the first step is performed before any of the other steps, but the other steps may be performed in any sequence unless a sequence is further specified within the other steps. For example, claim elements that recite “first A, then B, C, and D, and lastly E” shall be construed to mean step A should be first, step E should be last, but steps B, C, and D may be carried out in any sequence between steps A and E and the process of that sequence will still fall within the four corners of the claim.

Furthermore, in the claims provided herein, specified steps may be carried out concurrently unless explicit claim language requires that they be carried out separately or as parts of different processing operations. For example, a claimed step of doing X and a claimed step of doing Y may be conducted simultaneously within a single operation, and the resulting process will be covered by the claim. Thus, a step of doing X, a step of doing Y, and a step of doing Z may be conducted simultaneously within a single process step, or in two separate process steps, or in three separate process steps, and that process will still fall within the four corners of a claim that recites those three steps.

Similarly, except as explicitly required by claim language, a single substance or component may meet more than a single functional requirement, provided that the single substance fulfills the more than one functional requirement as specified by claim language.

All patents, patent applications, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Additionally, all claims in this application, and all priority applications, including but not limited to original claims, are hereby incorporated in their entirety into, and form a part of, the written description of the invention.

Applicant reserves the right to physically incorporate into this specification any and all materials and information from any such patents, applications, publications, scientific articles, web sites, electronically available information, and other referenced materials or documents. Applicant reserves the right to physically incorporate into any part of this document, including any part of the written description, the claims referred to above including but not limited to any original claims.

The pending claims are:

1. A computer-assisted method of generating a protonophore, the method requiring the use of a computer comprising a processor, the method comprising:
designing the protonophore;
calculating, using the processor, an estimated protonophoric activity across a biological membrane with a pH gradient for the protonophore;
producing the protonophore if the estimated protonophoric activity across the biological membrane with the pH gradient for the protonophore corresponds to an U₅₀ of about 20 μM or less; and
determining an uncoupling activity of the protonophore.

2. The method of claim 1, wherein the biological membrane with the pH gradient includes an inner membrane of a mitochondrion, a thylakoid membrane of a chloroplast, an outer membrane of an aerobic bacterium, or an outer membrane of an archaeum.

3. The method of claim 1, wherein the designing the protonophore comprises:
adding one or more hydroxyl or thiol groups to an aromatic or a heteroaromatic ring system or replacing one or more of ring atoms of the aromatic or heteroaromatic ring system with one or more unsubstituted acidic or basic nitrogen atoms to provide a first ionizable intermediate having a proportion of an unionized species and a proportion of an ionized species on a first side and on a second side of a biological membrane,
wherein the aromatic or the heteroaromatic ring system is unsubstituted or substituted with one or more oxygen atoms;
provided that if the proportion of the ionized species is less than about one thousand times greater than the proportion of the unionized species on either the first side or the second side of the biological membrane or that the proportion of the unionized species is less than about one thousand times greater than the proportion of the ionized species on either the first side or the second side of the biological membrane, then adding one or more acidity-modulating substituents directly to the aromatic or the heteroaromatic ring system of the first ionizable intermediate to provide a second ionizable intermediate having the proportion of the ionized species two or more times greater than the proportion of the unionized species on both the first and the second sides of the biological membrane or having the proportion of the unionized species two or more times greater than the proportion of the ionized species on both the first and the second sides of the biological membrane,
provided that the one or more acidity-modulating substituents do not comprise one or more nitro groups or one or more cyano groups; and
adding one or more lipophilicity-conferring substituents directly to the aromatic or the heteroaromatic ring system of the first ionizable intermediate or the second ionizable intermediate or to the one or more acidity-modulating substituents of the second ionizable intermediate to provide the protonophore,
wherein the protonophore exhibits a planar and a linear three-dimensional geometry, and provided that:
if the proportion of the unionized species of the protonophore is greater than the proportion of the ionized species of the protonophore, then the ionized species exhibits a greater degree of diffusibility across a biological membrane than the unionized species, or
if the proportion of the ionized species of the protonophore is greater than the proportion of the unionized species of the protonophore, then the unionized species exhibits a greater degree of diffusibility across the biological membrane than the ionized species.

4. The method of claim 3, wherein the one or more acidity-modulating substituents each independently include formyl or NH₂.

5. The method of claim 3, wherein the one or more lipophilicity-conferring substituents each independently include (C₁-C₁₂)alkyl, (C₁-C₁₂)alkenyl, (C₁-C₁₂)aldehyde, (C₁-C₁₂)alkoxy, (C₆-C₁₂)aryl, halogen, or haloalkyl.

6. The method of claim 1, wherein the calculating, using the processor, an estimated protonophoric activity across a biological membrane with a pH gradient for the protonophore comprises: calculating, using the processor, the estimated protonophoric activity across the biological membrane with the pH gradient for the protonophore as a function of an inverse of a first sum of a first resistance to diffusion across the biological membrane with the pH gradient for an unionized species of the protonophore and of a second resistance to diffusion across the biological membrane with the pH gradient for an ionized species of the protonophore; and comparing the estimated protonophoric activity across the biological membrane with the pH gradient for the protonophore with a second estimated protonophoric activity for a reference protonophore of known U₅₀.

7. The method of claim 6, wherein the determining the first resistance to diffusion across the biological membrane with the pH gradient for the unionized species of the protonophore and determining the second resistance to diffusion across the biological membrane with the pH gradient for the ionized species of the protonophore comprises:
calculating the first resistance to diffusion across the biological membrane with the pH gradient for the unionized species of the protonophore as an inverse function of a first permeability across the biological membrane with the pH gradient for the unionized species of the protonophore and a function of a first ratio of a first number of molecules of the unionized species of the protonophore at a steady-state condition on a first side of the biological membrane with the pH gradient from which the unionized species of the protonophore translocates over a second number of molecules of the ionized species of the protonophore at the steady-state condition on a second opposite side of the biological membrane with the pH gradient; and
calculating the second resistance to diffusion across the biological membrane with the pH gradient for the ionized species of the protonophore as an inverse function of a second permeability across the biological membrane with the pH gradient for the ionized species of the protonophore and a function of the first ratio.

8. The method of claim 1, wherein the determining the uncoupling activity of the protonophore includes measuring an increase of a rate of oxygen consumption in a preparation of isolated mitochondria, in a preparation of cells in culture, or in a preparation of tissues in culture, or measuring a bactericidal or a bacteriostatic effect, a fungicidal or a fungistatic effect, a herbicidal effect, or a pesticidal effect.

9. The method of claim 1, wherein the protonophore comprises a compound of Formula (I) 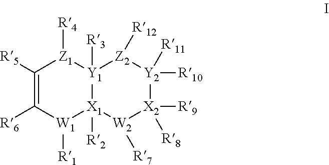
wherein:
W₁ is carbon, oxygen, sulfur, or nitrogen;
X₁, Y₁, and Z₁ are each independently carbon;
R′₁ is absent, hydroxyl, or thiol;
R′₂ is hydrogen, (C1-C6)aldehyde, (C1-C8)alkyl, acetyl, amino, (C1-C6)dialkylamino, or (C1-C8)alkenyl;
R′₃ is hydrogen, hydroxyl, thiol, (C1-C6)aldehyde, (C1-C8)alkyl, or (C1-C8)alkenyl;
R′₄ is hydrogen, (C1-C6)aldehyde, hydroxyl, (C1-C8)alkyl, (C1-C6)dialkylamino, (C1-C8)alkyl, acetyl, (C1-C8)alkenyl, CO(C1-C8)alkenyl, CO(C1-C8)alkyl, or CO-p-C₆H₅SH;
R′₅ is hydrogen, (C1-C6)aldehyde, (C1-C8)alkyl, thiol, acetyl, (C1-C8)alkenyl, or CO(C1-C8)alkenyl; and
R′₆ is hydrogen, amino, (C1-C6)dialkylamino, (C1-C6)aldehyde, (C1-C8)alkyl, (C1-C6)alkoxy, acetyl, (C1-C8)alkenyl, CO(C1-C8)alkenyl, or CO(C1-C8)alkyl,
provided that W₂, R′₇, X₂, R′₈, R′₉, Y₂, R′₁₀, R′₁₁, Z₂, and R′₁₂ are absent; or
wherein:
W₁, X₁, Y₁, and Z₁ are each independently carbon;
W₂ is oxygen, carbon, or nitrogen;
X₂ is carbon or nitrogen;
Y₂ and Z₂ are each independently carbon;
R′₁ is hydrogen, (C1-C6)aldehyde, (C1-C8)alkyl, acetyl, (C1-C8)alkenyl, CO(C1-C8)alkenyl, or CO(C1-C8)alkyl;
R′₂ and R′₃ are each independently absent;
R′₄ is hydrogen, (C1-C6)aldehyde, (C1-C8)alkyl, acetyl, or (C1-C8)alkenyl;
R′₅ is hydrogen, hydroxyl, thiol, amino, (C1-C8)alkyl, (C1-C6)aldehyde, acetyl, (C1-C8)alkylamino, (C1-C8)alkenyl, CO(C1-C8)alkenyl, or CO(C1-C8)alkyl;
R′₆ is hydrogen, hydroxyl, (C1-C6)aldehyde, thiol, (C1-C8)alkyl, amino, (C1-C8)alkylamino, acetyl, (C1-C8)alkenyl, CO(C1-C8)alkenyl, or CO(C1-C8)alkyl;
R′₇ is absent, hydrogen, (C1-C6)aldehyde, amino, or (C1-C8)alkylamino;
R′₈ is absent, hydrogen, acetyl, (C1-C8)alkyl, amino, (C1-C8)alkylamino, (C1-C8)alkenyl, or CO(C1-C8)alkenyl or R′₈, R′₉, R′₁₀ and R′₁₁ together form two carbon atoms of an unsubstituted or substituted(C1-C12)aryl or two carbon atoms of an unsubstituted or substituted(C1-C12)hetero aromatic;
R′₉ and R′₁₁ are each independently absent or hydrogen;
R′₁₀ is hydrogen, hydroxyl, thiol, amino, (C1-C8)alkylamino, (C1-C8)alkyl, or (C1-C8)alkenyl;
R′₁₂ is carbonyl, hydrogen, amino, (C1-C8)alkylamino, or (C1-C8)alkyl; or
wherein:
W₁ is oxygen, sulfur, carbon, or nitrogen;
X₁ and Y₁ are each independently carbon or nitrogen;
Z₁ is absent;
W₂ and Z₂ are each independently hydrogen;
R′₁ is absent, hydrogen, or carbonyl;
R′₂ is absent, hydrogen, thiol, hydroxyl, (C1-C8)alkyl, acetyl, (C1-C8)alkenyl, CO(C1-C8)alkenyl, CO(C1-C8)alkyl; or CO—(C1-C6)aryl;
R′₃ is absent, hydrogen, thiol, hydroxyl, (C1-C6)aldehyde, (C1-C8)alkyl, acetyl, CO(C1-C8)alkenyl, or CO(C1-C8)alkyl;
R′₄ is absent;
R′₅ is hydrogen, (C1-C8)alkyl, (C1-C8)alkenyl, or CO(C1-C8)alkenyl; or R′₅ and R′₆ together form two carbon atoms of an unsubstituted or substituted(C1-C12)aryl;
R′₆ is hydroxyl, (C1-C6)aldehyde, acetyl, (C1-C8)alkyl, (C1-C8)alkenyl, CO(C1-C8)alkenyl, or CO(C1-C8)alkyl;
W₂, X₂, Y₂, Z₂, R′₇, R′₈, R′₉, R′₁₀, R′₁₁, and R′₁₂ is absent; and
R′₂, R′₃, Z₁, W₂, X₂, Y₂, Z₂, R′₇, R′₈, R′₉, R′₁₀, R′₁₁, and R′₁₂ together form two carbon atoms of an unsubstituted or substituted(C1-C12)aryl; or
wherein:
W₁ is oxygen;
X₁, Y₁, and Z₁ are each independently carbon;
R′₁, R′₂, and R′₃ are each independently absent;
R′₄ and R′₆ are each independently (C1-C8)alkyl;
R′₅ is hydroxyl;
W₂ and Z₂ are each independently CH;
X₂ and Y₂ are each independently carbon;
R′₇ and R′₁₂ are each independently hydrogen; and
R′₈, R′₉, R′₁₀ and R′₁₁ together form two carbon atoms of an unsubstituted or substituted(C1-C12)heteroaromatic, or a pharmaceutically acceptable salt, solvate, or prodrug thereof.

10. A method of treating a disorder, disease, or condition benefiting from a protonophore-induced uncoupling of mitochondrial oxidative phosphorylation in a patient in need thereof comprising administering a composition comprising a protonophore of Formula (I) 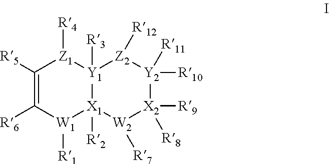
wherein:
W₁ is carbon, oxygen, sulfur, or nitrogen;
X₁, Y₁, and Z₁ are each independently carbon;
R′₁ is absent, hydroxyl, or thiol;
R′₂ is hydrogen, (C1-C6)aldehyde, (C1-C8)alkyl, acetyl, amino, (C1-C6)dialkylamino, or (C1-C8)alkenyl;
R′₃ is hydrogen, hydroxyl, thiol, (C1-C6)aldehyde, (C1-C8)alkyl, or (C1-C8)alkenyl;
R′₄ is hydrogen, (C1-C6)aldehyde, hydroxyl, (C1-C8)alkyl, (C1-C6)dialkylamino, (C1-C8)alkyl, acetyl, (C1-C8)alkenyl, CO(C1-C8)alkenyl, CO(C1-C8)alkyl, or CO-p-C₆H₅SH;
R′₅ is hydrogen, (C1-C6)aldehyde, (C1-C8)alkyl, thiol, acetyl, (C1-C8)alkenyl, or CO(C1-C8)alkenyl; and
R′₆ is hydrogen, amino, (C1-C6)dialkylamino, (C1-C6)aldehyde, (C1-C8)alkyl, (C1-C6)alkoxy, acetyl, (C1-C8)alkenyl, CO(C1-C8)alkenyl, or CO(C1-C8)alkyl,
provided that W₂, R′₇, X₂, R′₈, R′₉, Y₂, R′₁₀, R′₁₁, Z₂, and R′₁₂ are absent; or
wherein:
W₁, X₁, Y₁, and Z₁ are each independently carbon;
W₂ is oxygen, carbon, or nitrogen;
X₂ is carbon or nitrogen;
Y₂ and Z₂ are each independently carbon;
R′₁ is hydrogen, (C1-C6)aldehyde, (C1-C8)alkyl, acetyl, (C1-C8)alkenyl, CO(C1-C8)alkenyl, or CO(C1-C8)alkyl;
R′₂ and R′₃ are each independently absent;
R′₄ is hydrogen, (C1-C6)aldehyde, (C1-C8)alkyl, acetyl, or (C1-C8)alkenyl;
R′₅ is hydrogen, hydroxyl, thiol, amino, (C1-C8)alkyl, (C1-C6)aldehyde, acetyl, (C1-C8)alkylamino, (C1-C8)alkenyl, CO(C1-C8)alkenyl, or CO(C1-C8)alkyl;
R′₆ is hydrogen, hydroxyl, (C1-C6)aldehyde, thiol, (C1-C8)alkyl, amino, (C1-C8)alkylamino, acetyl, (C1-C8)alkenyl, CO(C1-C8)alkenyl, or CO(C1-C8)alkyl;
R′₇ is absent, hydrogen, (C1-C6)aldehyde, amino, or (C1-C8)alkylamino;
R′₈ is absent, hydrogen, acetyl, (C1-C8)alkyl, amino, (C1-C8)alkylamino, (C1-C8)alkenyl, or CO(C1-C8)alkenyl or R′₈, R′₉, R′₁₀ and R′₁₁ together form two carbon atoms of an unsubstituted or substituted(C1-C₁₂)aryl or two carbon atoms of an unsubstituted or substituted(C1-C₁₂)heteroaromatic;
R′₉ and R′₁₁ are each independently absent or hydrogen;
R′₁₀ is hydrogen, hydroxyl, thiol, amino, (C1-C8)alkylamino, (C1-C8)alkyl, or (C1-C8)alkenyl;
R′₁₂ is carbonyl, hydrogen, amino, (C1-C8)alkylamino, or (C1-C8)alkyl; or
wherein:
W₁ is oxygen, sulfur, carbon, or nitrogen;
X₁ and Y₁ are each independently carbon or nitrogen;
Z₁ is absent;
W₂ and Z₂ are each independently hydrogen;
R′₁ is absent, hydrogen, or carbonyl;
R′₂ is absent, hydrogen, thiol, hydroxyl, (C1-C8)alkyl, acetyl, (C1-C8)alkenyl, CO(C1-C8)alkenyl, CO(C1-C8)alkyl; or CO—(C1-C6)aryl;
R′₃ is absent, hydrogen, thiol, hydroxyl, (C1-C6)aldehyde, (C1-C8)alkyl, acetyl, CO(C1-C8)alkenyl, or CO(C1-C8)alkyl;
R′₄ is absent;
R′₅ is hydrogen, (C1-C8)alkyl, (C1-C8)alkenyl, or CO(C1-C8)alkenyl; or R′₅ and R′₆ together form two carbon atoms of an unsubstituted or substituted(C1-C₁₂)aryl;
R′₆ is hydroxyl, (C1-C6)aldehyde, acetyl, (C1-C8)alkyl, (C1-C8)alkenyl, CO(C1-C8)alkenyl, or CO(C1-C8)alkyl;
W₂, X₂, Y₂, Z₂, R′₇, R′₈, R′₉, R′₁₀, R′₁₁, and R′₁₂ is absent; and
R′₂, R′₃, Z₁, W₂, X₂, Y₂, Z₂, R′₇, R′₈, R′₉, R′₁₀, R′₁₁, and R′₁₂ together form two carbon atoms of an unsubstituted or substituted(C1-C₁₂)aryl; or
wherein:
W₁ is oxygen;
X₁, Y₁, and Z₁ are each independently carbon;
R′₁, R′₂, and R′₃ are each independently absent;
R′₄ and R′₆ are each independently (C1-C8)alkyl;
R′₅ is hydroxyl;
W₂ and Z₂ are each independently CH;
X₂ and Y₂ are each independently carbon;
R′₇ and R′₁₂ are each independently hydrogen; and
R′₈, R′₉, R′₁₀ and R′₁₁ together form two carbon atoms of an unsubstituted or substituted(C1-C12)heteroaromatic, or a pharmaceutically acceptable salt, solvate, or prodrug thereof.

11. The method of claim 10, wherein the disorder, disease or condition comprises insulin resistance, impaired glucose tolerance, Type I diabetes, Type II diabetes, fatty liver disease, lipid accumulation in striated muscle, hyperglycemia, hyperinsulinemia, cancer, or a combination thereof.

12. The method of claim 10, wherein the protonophore of Formula (I) is represented by a protonophore of Formula (II) 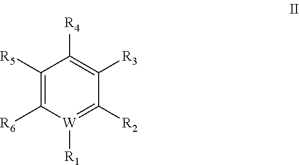
wherein:
W is C, O, S, or N;
R₁ is Absent, OH, or SH;
R₂ is Hydrogen, NH₂, CH3, C(CH₃)₃, COCH3, CHO, CH═CH₂, CH═CHCH₃, CH═CHCH═CH₂, (CH═CH)₂(CH₃), (CH═CH)₂CH═CH₂, or (CH═CH)₂CH═CH₂;
R₃ is Hydrogen, OH, SH, CH₃, CHO, CH═CH₂, CH═C(CH₃)₂, (CH═CH)₂(CH₃), or (CH═CH)₃(CH₃);
R₄ is CHO, CH₃, C(CH₃)₃, N(CH₃)₂, COCH₃, CH═CH₂, CH═CHCH₃, CH═CH₂CH═CH₂, (CH═CH)₂(CH₃), COCH═CH₂, CO(CH═CH)₂CH═CH₂, CO(CH═CH)₃(CH₃), or CO-p-C₆H₅SH;
R₅ is Hydrogen, CHO, CH₃, SH, COCH₃, C(CH₃)₃, CH═CH₂, CH═CHCH₃, CH═C(CH₃)₂ (CH═CH)₄(CH₃), or COCH═CH₂; and
R₆ is Hydrogen, NH₂, N(CH₃)₂, CHO, CH₃, OCH₃, COCH₃, CH═CH₂, CH═C(CH₃)₂, COCH═CH₂, or CO(CH₂)₇CH₃, or a pharmaceutically acceptable salt, solvate, or prodrug thereof.

13. The method of claim 12, wherein the protonophore is:
1,3-dihydroxy, 2-(propen-1-yl), 3,6-diformyl, benzene,
1,3-dihydroxy, 4,6-di(prop-2-en-1-one), benzene,
1,3 dihydroxy, 2,5-diethenyl, 4,6-diacetyl, benzene,
1,3-dihydroxy, 2-((1E)-buta-1,3-dien-1-yl), 4,6-acetyl, benzene,
2,4-diacetyl, 3-((1E,3E,5E)-hepta-1,3,5-trien-1-yl), thiophenol,
2,4,6-triformyl, 3-methyl, 5-tert-butyl, thiophenol,
2,4-diformyl, 3-((1E,3E)-penta-1,3-dien-1-yl), thiophenol,
3,5-diformyl, 4-((1E,3E)-penta-1,3-dien-1-yl), thiophenol,
2,4,6-triformyl, 3-((1E,3E,5E)-hepta-1,3,5-trien-1-yl), thiophenol,
2,6-diformyl, 4-((2E,4E,6E)-octa-2,4,6-trien-1-one), thiophenol,
2-formyl, 4-((2E,4E,6E)-hepta-2,4,6-trien-1-one), thiophenol,
2-acetyl, 4-(hexan-1-one), thiophenol,
2-ethenyl, 3-sulfanyl, 5-(prop-2-en-1-one), thiophenol,
2-((1E,3E)-hexa-1,3,5-trien-1-yl), 3-sulfanyl, 4,6-diacetyl, thiophenol,
2,5,6-trimethyl, 3-sulfanyl, 4-acetyl, thiophenol,
2-methyl, 3-sulfanyl, 4-formyl, 6-ethenyl, thiophenol,
2-((1E,3E)-penta-1,3-dien-1-yl), 3-sulfanyl, 5-acetyl, thiophenol,
2,5,6-trimethyl, 3-sulfanyl, 4-formyl, thiophenol,
4-[(4-sulfanylphenyl)carbonyl]benzenethiol,
1,3,5-trisulfanyl, 2,4-dimethyl, 6-methoxy, benzene,
1,3,5-trisulfanyl, 2,4-dimethyl, benzene,
1,3,5-trisulfanyl, 4-(propen-1-yl), benzene,
3-hydroxy, 4-[(1E)-buta-1,3-dien-1-yl], 5-methyl, pyrilium,
3-hydroxy, 4,5-diethenyl, 6-methyl, pyrilium,
3-hydroxy, 4,5-diethenyl, pyrilium,
3-hydroxy, 4-(propen-1-yl), 5-ethenyl, pyrilium,
2,4-dimethyl, 3-hydroxy, 5-[(1E,3E,5E,7E)-nona-1,3,5,7-tetraen-1-yl], 6-formyl, thiopyran,
2,4-tert-butyl, 3-hydroxy, 5-methyl, 6-formyl, thiopyran,
2,4,5-tri-(propen-1-yl), 3-hydroxy, 6-formyl, 27-4-thiopyran,
2,4-dimethyl, 3-hydroxy, 6-(nonan-1-one), thiopyran,
4-N,4-N-dimethyl, 2,4,6-triamine, 3,5-di-(2-methylpropen-1-yl), pyridine,
2-N,2-N,4-N,4-N,6-N,6-N-hexamethy, 2,4,6-triamine, 3,5-dimethyl, pyridine,
2,6-di-(2-methylpropen-1-yl), 3,5-diethenyl, 4-hydroxy, pyridine, or
2,3,4,5,6-pentaethenyl, pyridine.

14. The method of claim 12, wherein the protonophore is

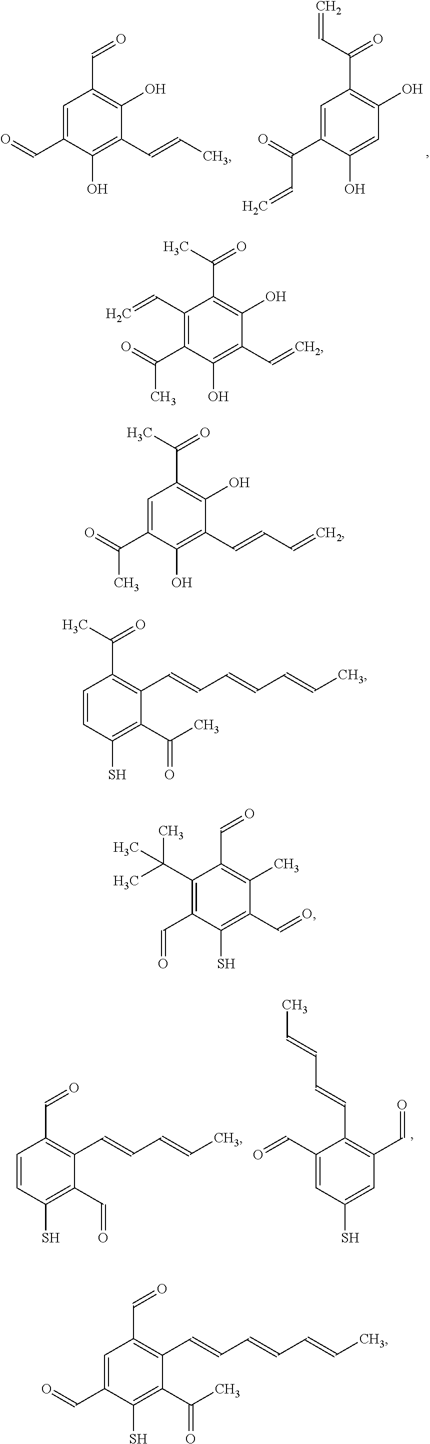

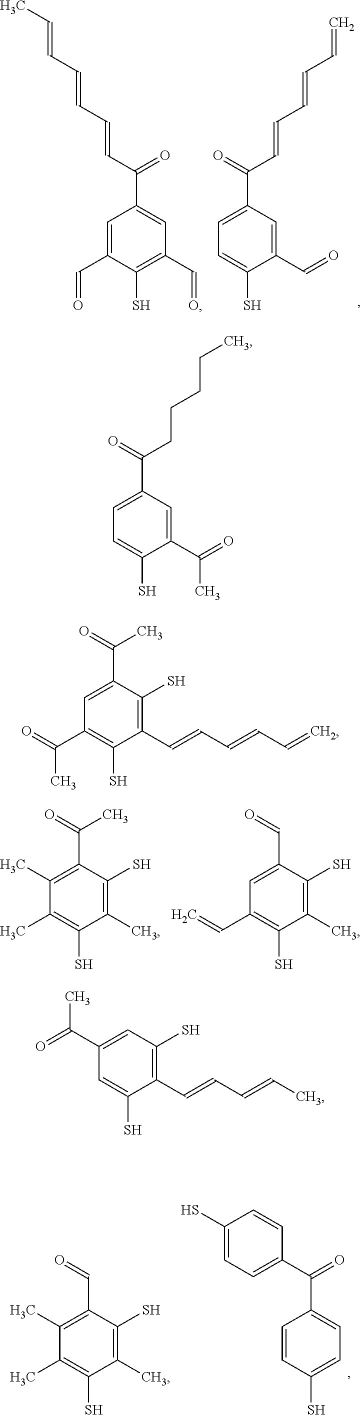

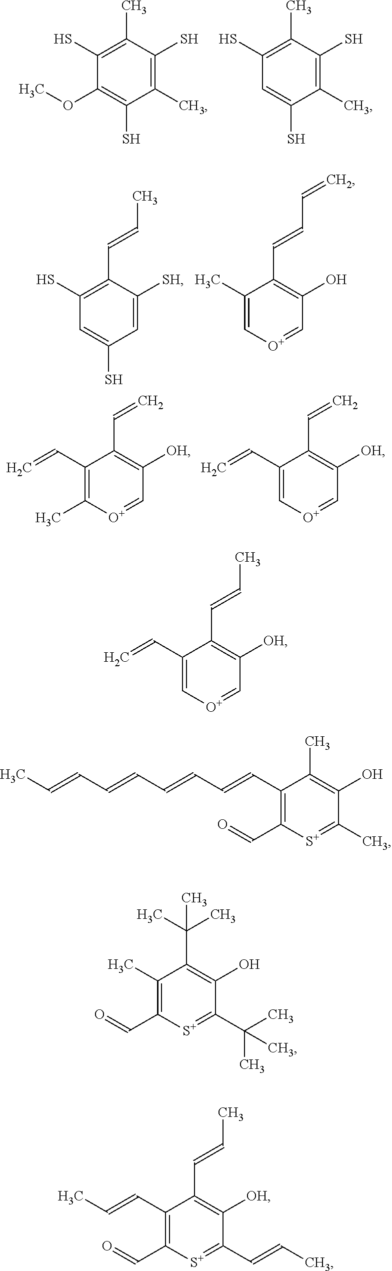

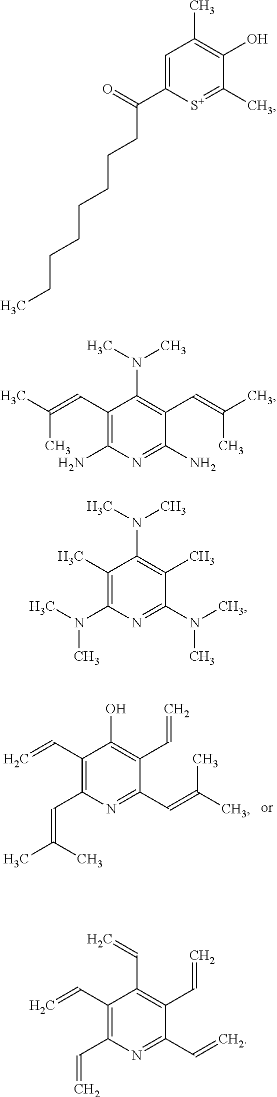 [SMILES: CCCCCCCCC(=O)c1cc(C)c(O)c(C)[s+]1 ; CC(C)=Cc1c(N)nc(N)c(C=C(C)C)c1N(C)C ; Cc1c(N(C)C)nc(N(C)C)c(C)c1N(C)C ; C=Cc1c(C=C(C)C)nc(C=C(C)C)c(C=C)c1O ; "or" ; C=Cc1nc(C=C)c(C=C)c(C=C)c1C=C]

15. The method of claim 10, wherein the protonophore of Formula (I) is represented by a protonophore of Formula (III) 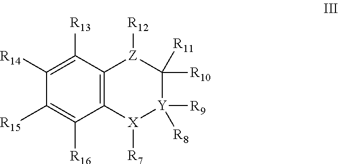
wherein:
X is O, C, or N;
Y is C or N;
Z is C;
R₇ is Absent, Hydrogen, CHO, ═O, NH₂, or NHCH₃, provided that when X is O, then R₇ is Absent, or when X is C, then R₇ is Hydrogen, CHO, ═O, NH₂, or NHCH₃ or when X is N, then R₇ is Absent or Hydrogen;
R₈ is Hydrogen, CH₃, NH₂, NHCH₃, COCH₃, CH═CH₂, CH═CHCH₃, CH═C(CH₃)₂, CH═CHCH═CH₂, (CH═CH)₂CH═CH₂, (CH═CH)₃CH₃, COCH═CH₂, COCH═CH(CH₃), COCH═C(CH₃)₂, or R₈, R₉, R₁₀ and R₁₁ together form the 3 and 4 carbon atoms of 2-carboxaldehyde-phenol, the 3 and 4 carbon atoms of 2-carboxaldehyde-6-methyl-phenol, the 3 and 4 carbon atoms of 2-acetyl-6-methyl-phenol, the 4 and 5 carbon atoms of 2-carboxaldehyde-phenol, the 3 and 4 carbon atoms of 2-carboxaldehyde-phenol, the 3 and 4 carbon atoms of 2-carboxaldehyde-6-methyl-phenol, the 3 and 4 carbon atoms of 2-carboxaldehyde-phenol, the 1 and 2 carbon atoms of phenyl, or the 2 and 3 carbon atoms of [2,3-b]1-H pyrole;
R₉ and R₁₁ are each independently Absent or Hydrogen;
R₁₀ is Hydrogen, OH, SH, NH₂, CH₃, NHCH₃, CH═CH₂, or CH═CH(CH₃);
R₁₂ is ═O, Hydrogen, NH₂, NHCH₃, or CH₃;
R₁₃ is Hydrogen, CHO, CH₃, COCH₃, or CH═CH₂;
R₁₄ is Hydrogen, OH, SH, NH₂, CH₃, CHO, COCH₃, NHCH₃, CH═CH₂, CH═CH(CH₃), C(CH₃)═CH(CH₃), CH═CHCH═CH₂, (CH═CH)₂CH═CH₂, (CH═CH)₃CH₃, COCH═CH₂, COCH═CHCH₃, CO(CH₂)2CH3, or CO(CH═CH)₂CH₃;
R₁₅ is Hydrogen, OH, CHO, SH, CH₃, NH₂, NHCH₃, COCH₃, CH═CH₂, CH═CHCH₃, CH═CHCH═CH₂, (CH═CH)₂(CH₃), COCH═CHCH₃, CO(CH₂)₃CH₃, or CO(CH₂)₄CH₃; and
R₁₆ is Hydrogen, CHO, CH₃, COCH₃, CH═CH₂, CH═CH(CH₃), (CH═CH)₂CH₃, (CH═CH)₂CH═CH₂, (CH═CH)₃CH₃, COCH═CH₂, COCH═CHCH₃, or CO(CH₂)₃CH₃, or a pharmaceutically acceptable salt, solvate, or prodrug thereof.

16. The method of claim 15, wherein the protonophore is
2-(but-3-en-2-one), 3-hydroxy, 5,7-dimethyl, chromone,
2-(prop-2-en-1-one), 3-hydroxy, 6,7-dimethyl, chromone,
2-(2-methyl-prop-2-en-1-one), 3-hydroxy, 7-methyl, chromone,
2-acetyl, 3-hydroxy, 5,7-dimethyl, 6-ethenyl, chromone,
3-hydroxy, 6-(propen-1-yl)₅7-(but-2-en-1-one), chromone,
2-((1E)-buta-1,3-dien-1-yl), 3-hydroxy, 5,6-dimethyl, 8-acetyl, chromone,
2-(prop-2-en-1-one), 3-hydroxy, 6-(propen-1-yl), chromone,
2-(but-2-en-1-one), 3-hydroxy, 6-ethenyl, chromone,
2,5-dimethyl, 6-((2E)-but-2-en-2-yl), 8-acetyl, chromone,
2,3,5-trimethyl, 6,8-diformyl, 7-hydroxy, chromone,
2,3-dimethyl, 6-(prop-2-en-1-one), 7-hydroxy, 8-acetyl, chromone,
2,3,5-trimethyl, 6,8-diformyl, 7-hydroxy, chromone,
2,3-dimethyl, 6-(prop-2-en-1-one), 7-hydroxy, 8-acetyl, chromone,
2-(propen-1-yl), 3-methyl, 6,8-diacetyl, 7-hydroxy, chromone,
2-(propen-1-yl), 6-acetyl, 7-hydroxy, 8-ethenyl, chromone,
2,3-dimethyl, 6-ethenyl, 7-hydroxy, 8-formyl, chromone,
6-(prop-2-en-1-one), 7-hydroxy, 8-ethenyl, chromone,
2,3-dimethyl, 6-formyl, 7-hydroxy, 8-ethenyl, chromone,
2,8-diethenyl, 3-methyl, 6-acetyl, 7-hydroxy, chromone,
3,6-diethenyl, 7-hydroxy, 8-formyl, chromone,
2,3-diethenyl, 6-acetyl, 7-hydroxy, 8-methyl, chromone,
3,8-diethenyl, 6-formyl, 7-hydroxy, chromone,
3-methyl, 7-hydroxy, 8-(but-2-en-1-one), chromone,
2-((1E)-buta-1,3-dien-1-yl), 3-methyl, 6-acetyl, 7-hydroxy, chromone,
3-methyl, 6-(propen-1-yl), 7-hydroxy, 8-acetyl, chromone,
2-((1E)-buta-1,3-dien-1-yl), 3-methyl, 7-hydroxy, 8-acetyl, chromone,
3-methyl, 6-(butan-1-one), 7-hydroxy, chromone,
6-acetyl, 7-hydroxy, 8-((1E,3E)-penta-1,3-dien-1-yl), chromone,
2-ethenyl, 3,7-dihydroxy, 6-(prop-2-en-1-one), 8-methyl, chromone,
2-(2-methylprop-1-en-1-yl), 3,7-dihydroxy, 6-acetyl, 8-methyl, chromone,
2,6,8-triethenyl, 3,7-dihydroxy, chromone,
2,6-di-(propen-1-yl), 3,7-dihydroxy, chromone,
2,3,5-trimethyl, 6,8-diformyl, 7-hydroxy, dihydrochromone,
6-((2E,4E)-hexa-2,4-dien-1-one), 7-hydroxy, 8-acetyl, dihydrochromone,
6-formyl, 7-hydroxy, 8-((1E,3E,5E)-hexa-1,3,5-trien-1-yl), dihydrochromone,
6-(prop-2-en-1-one), 7-hydroxy, 8-(propen-1-yl), dihydrochromone,
6-((1E,3E,5E)-hexa-1,3,5-trien-1-yl), 7-hydroxy, 8-acetyl, dihydrochromone,
6-(but-2-en-1-one), 7-hydroxy, 8-(prop-2-en-1-one), dihydrochromone,
6-formyl, 7-hydroxy, 8-(pentan-1-one), dihydrochromone,
3,6-dihydroxy-9-oxoxanthene-4,5-dicarbaldehyde,
4,7-diacetyl-3,6-dihydroxy-2-methylxanthen-9-one,
2-acetyl, 3-sulfanyl, 6-((1E)-buta-1,3-dien-1-yl), chromone,
2-(prop-2-en-1-one), 3-sulfanyl, 6-methyl, chromone,
2-methyl, 3-sulfanyl, 7-(pentan-1-one), chromone,
2,3-diethenyl, 6-formyl, 7-sulfanyl, 8-methyl, chromone,
2-ethenyl, 5,8-dimethyl, 6-formyl, 7-sulfanyl, chromone,
2-ethenyl, 5,8-dimethyl, 6-formyl, 7-sulfanyl, chromone,
2-(propen-1-yl), 3-ethenyl, 6-methyl, 7-sulfanyl, 8-acetyl, chromone,
2-(propen-1-yl), 3-ethenyl, 5-methyl, 6,8-diformyl, 7-sulfanyl, chromone,
6-formyl, 7-sulfanyl, 8-((1E,3E,5E)-hepta-1,3,5-trien-1-yl), dihydrochromone,
6-((1E,3E,5E)-hepta-1,3,5-trien-1-yl), 7-sulfanyl, 8-formyl dihydrochromone,
6-(pentan-1-one), 7-sulfanyl, 8-methyl, dihydrochromone,
6-methyl, 7-sulfanyl, 8-(pentan-1-one), dihydrochromone,
2,3,8-trimethyl, 5,7-diformyl, 6-hydroxy, 1,4-naphtoquinone,
2,3-di-(propen-1-yl), 6-hydroxy, 7-acetyl, 1,4-naphtoquinone,
2,3,5,8-tetramethyl, 6-hydroxy, 7-acetyl, 1,4-naphtoquinone,
2,5-dimethyl, 3-(propen-1-yl), 6-hydroxy, 7-acetyl, 1,4-naphtoquinone,
2-(propen-1-yl), 3,5-dimethyl, 6-hydroxy, 7-acetyl, 1,4-naphtoquinone,
2,3,7,8-tetramethyl, 5-acetyl, 6-hydroxy, 1,4-naphtoquinone,
2,3,8-triethenyl, 5-acetyl, 6-hydroxy, 1,4-naphtoquinone,
2,3-diethenyl, 5,7-diacetyl, 6-hydroxy, 8-methyl, 1,4-naphtoquinone,
2,8-diethenyl, 5,7-diformyl, 6-hydroxy, 1,4-naphtoquinone,
2-(propen-1-yl), 5,7-acetyl, 6-hydroxy, 8-ethenyl, 1,4-naphtoquinone,
1,3-diacetyl, 2-hydroxy, anthraquinone,
1,3-formyl, 2-hydroxy, anthraquinone,
2,6-dihydroxy, 3,7-diformyl, anthraquinone,
2,6-dihydroxy, 1,5-diformyl, anthraquinone,
2,3,5,8-tetramethyl, 6-sulfanyl, 7-formyl, 1,4-naphtoquinone,
2-((1E,3E,5E)-hepta-1,3,5-trien-1-yl), 6-sulfanyl, 7-acetyl, 1,4-naphtoquinone,
2-((1E,3E,5E)-hepta-1,3,5-trien-1-yl), 6-sulfanyl, 7-formyl, 1,4-naphtoquinone,
2-(propen-1-yl), 6-sulfanyl, 7-(but-2-en-1-one), 1,4-naphtoquinone,
2,3-dimethyl, 6-sulfanyl, 7-ethenyl, 1,4-naphtoquinone,
2-(propen-1-yl), 6-sulfanyl, 7-ethenyl, 1,4-naphtoquinone,
2-ethenyl, 6-sulfanyl, 7-(propen-1-yl), 1,4-naphtoquinone,
3-ethenyl, 6-sulfanyl, 7-(propen-1-yl), 1,4-naphtoquinone,
3-(propen-1-yl), 6-sulfanyl, 7-ethenyl, 1,4-naphtoquinone,
2-((1E,3E,5E)-hexa-1,3,5-trien-1-yl), 6-sulfanyl, 1,4-naphtoquinone,
6-sulfanyl, 7-(hexan-1-one), 1,4-naphtoquinone,
2-sulfanyl, anthracene-9,10-dione,
3-hydroxy, 6,7-dimethyl, chromenylium,
3-hydroxy, 2,6,7-trimethyl, chromenylium,
3-hydroxy, 6-ethenyl, chromenylium,
2-methyl, 3-hydroxy, 6-ethenyl, chromenylium,
3-hydroxy, 7-ethenyl, chromenylium,
2-methyl, 3-hydroxy, 7-ethenyl, chromenylium,
2-(propen-1-yl), 4-hydroxy, chromenylium,
4-hydroxy, 7-ethenyl, chromenylium,
7-ethenyl, 8-hydroxy, chromenylium,
2-methyl, 7-ethenyl, 8-hydroxy, chromenylium,
3,6-dihydroxy, 5-methyl, 7-ethenyl, chromenylium,
3,6-dihydroxy, 5,7,8-trimethyl, chromenylium,
2-methyl, 3,6-dihydroxy, 7-(propen-1-yl), chromenylium,
2,4,7-triamine, 3,5,6,8-tetraethenyl, quinoline,
2-N,4-N,7-N-trimethyl, 2,4,7-triamine, 3,5,6,8-tetramethyl, quinoline,
2,5,8-triamine, 3,4,7-trimethyl, 6-((1E)-buta-1,3-dien-1-yl), isoquinoline,
N-5-methyl, 2,5,8-triamine, 3,7-dimethyl, 4,6-diethenyl, isoquinoline,
N-2,N-8-methyl, 2,5,8-triamine, 3,4,6,7-tetramethyl, isoquinoline,
5,7-diethenyl-6-methyl-1H-naphtho[2,3-b]pyrrole-9-carbaldehyde,
6,8-dimethyl, 7-((1E,3E)-penta-1,3-dien-1-yl), 1-H-quinolin-4-one, or
6,8-dimethyl, 7-((1E,3E)-penta-1,3-dien-1-yl), 1-H-quinolin-4-one.

17. The method of claim 15, wherein the protonophore is

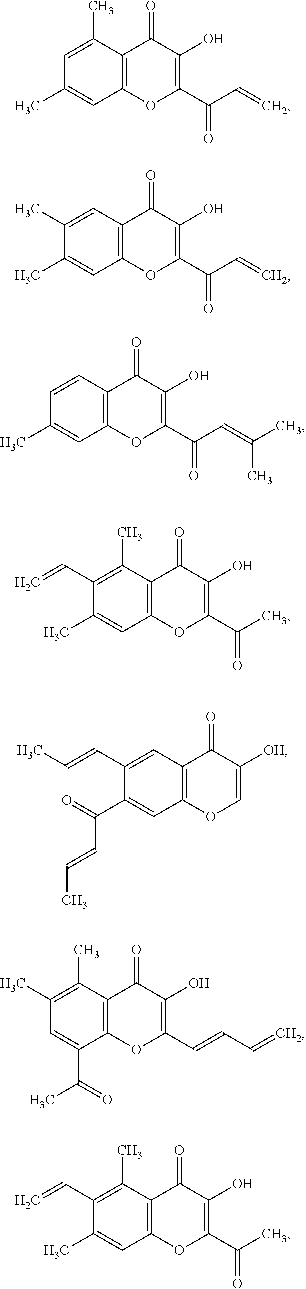

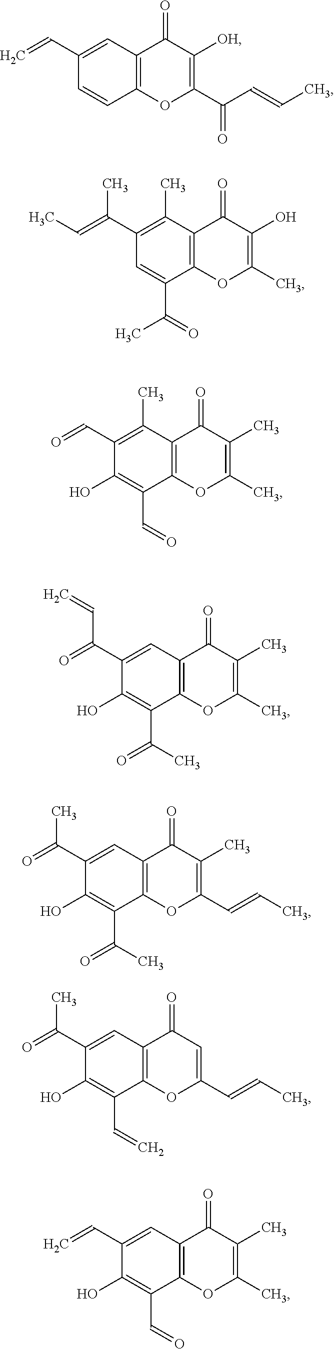

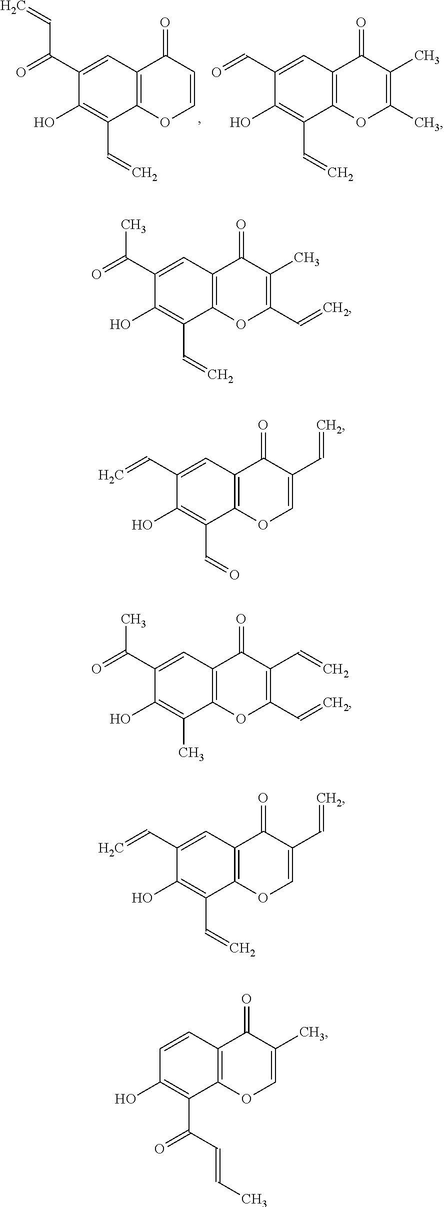

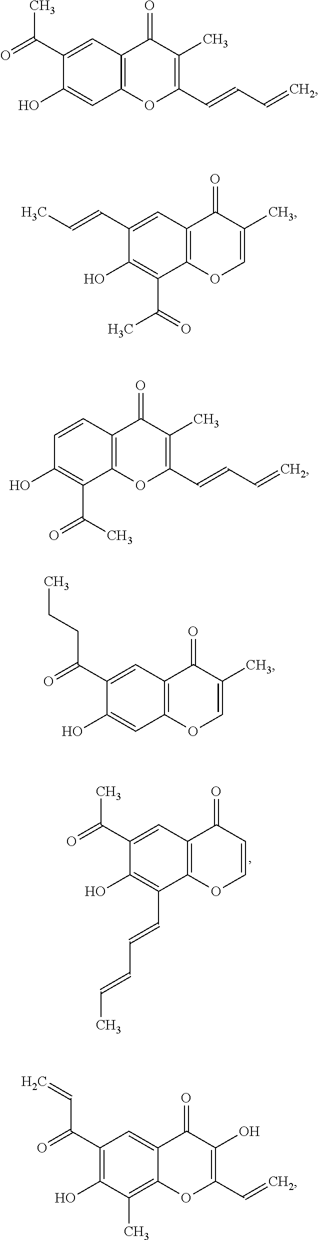

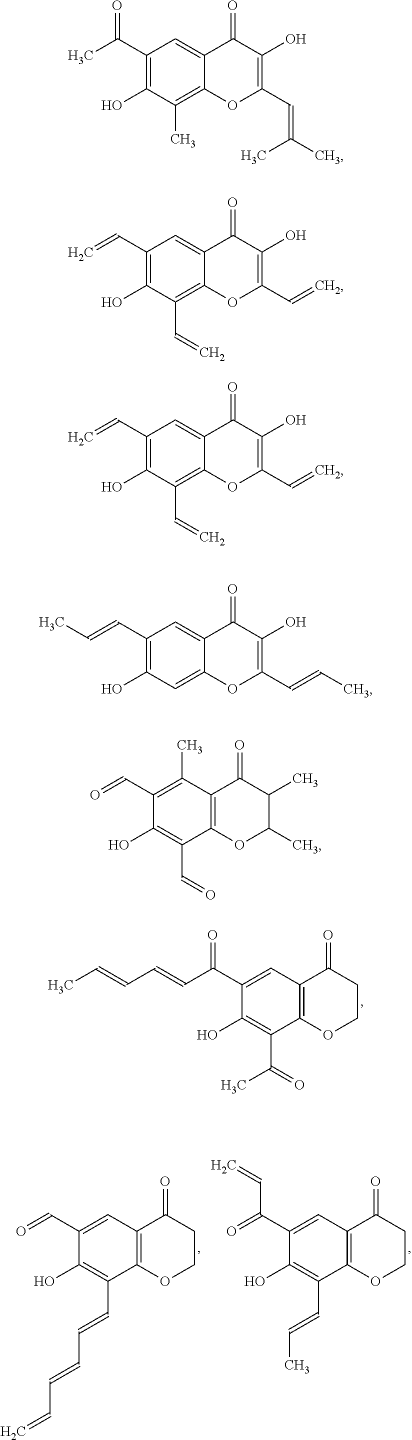

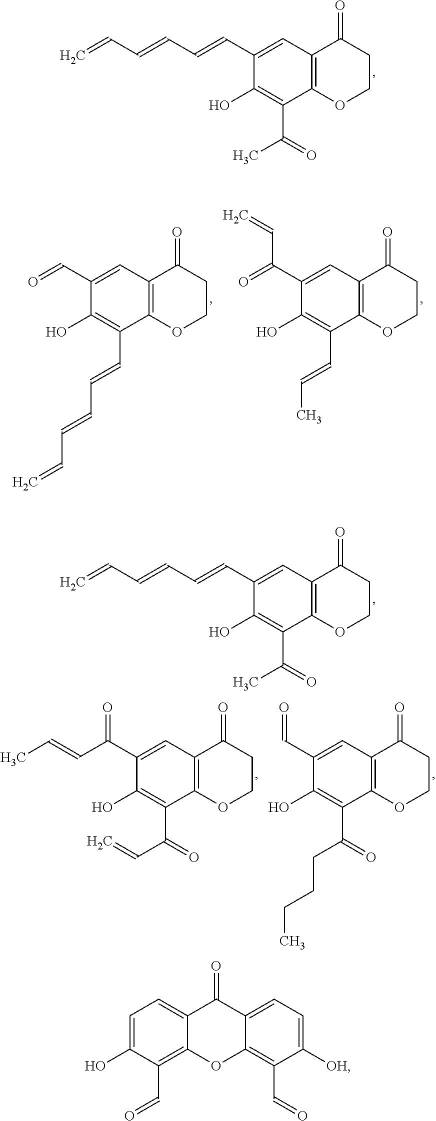

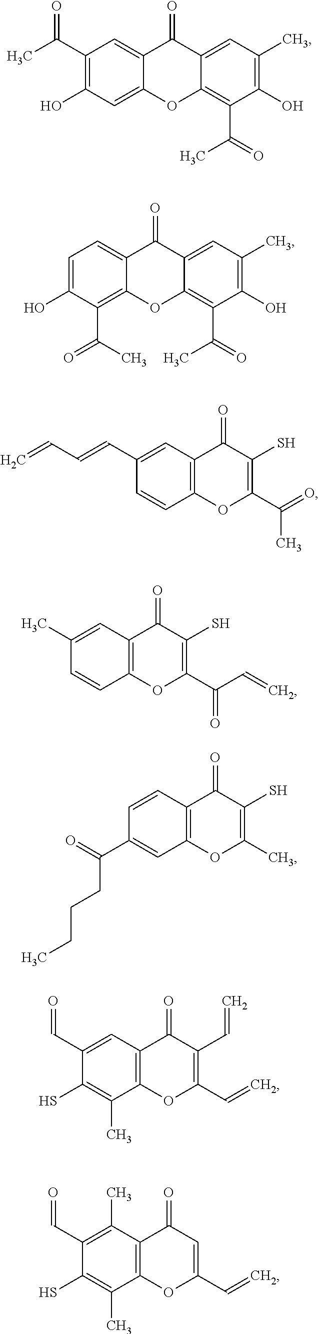

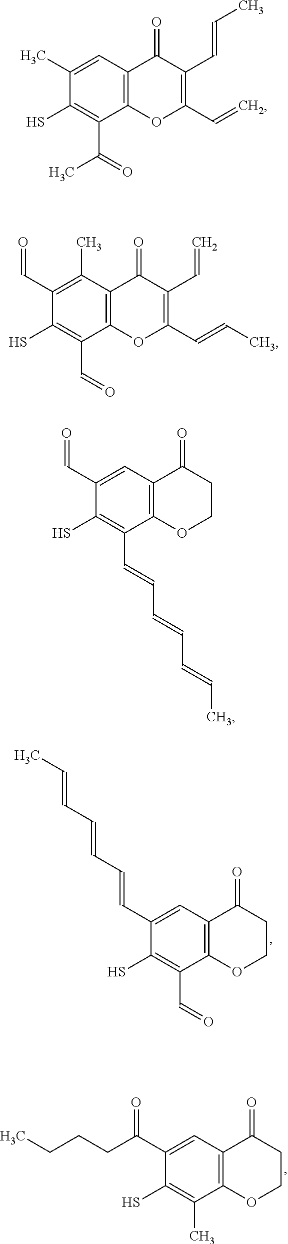

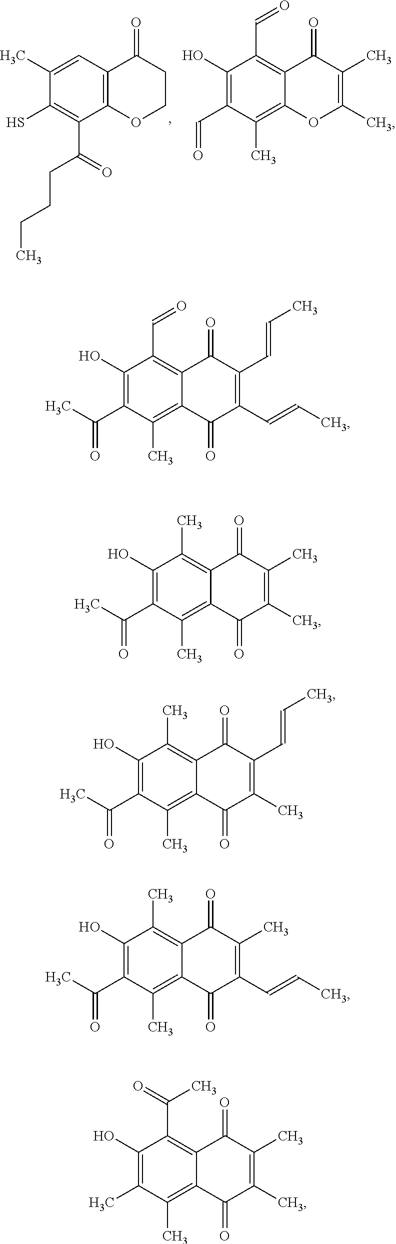

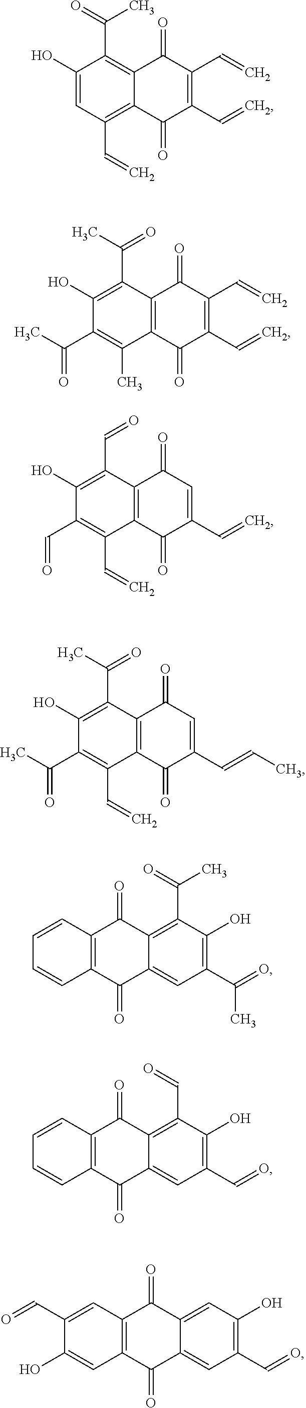

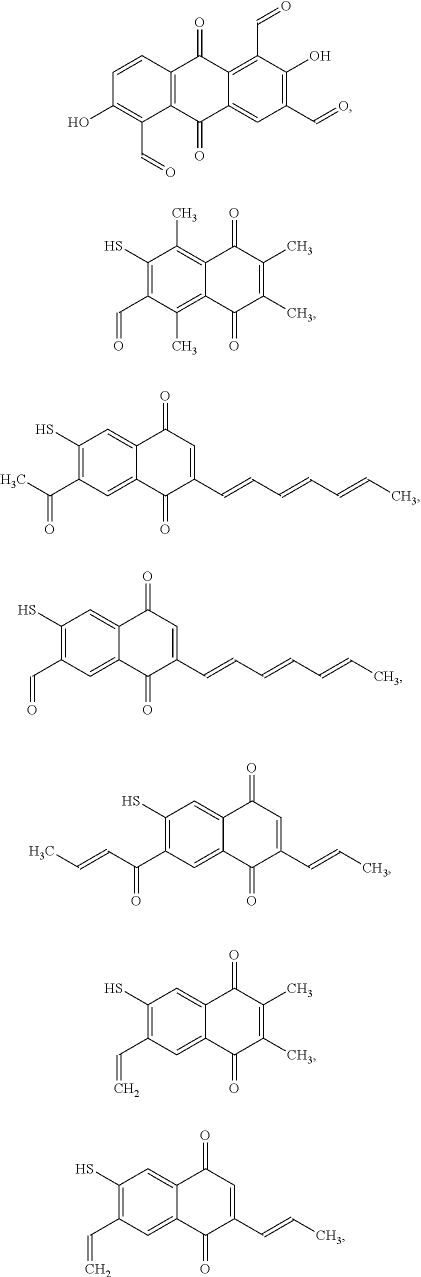

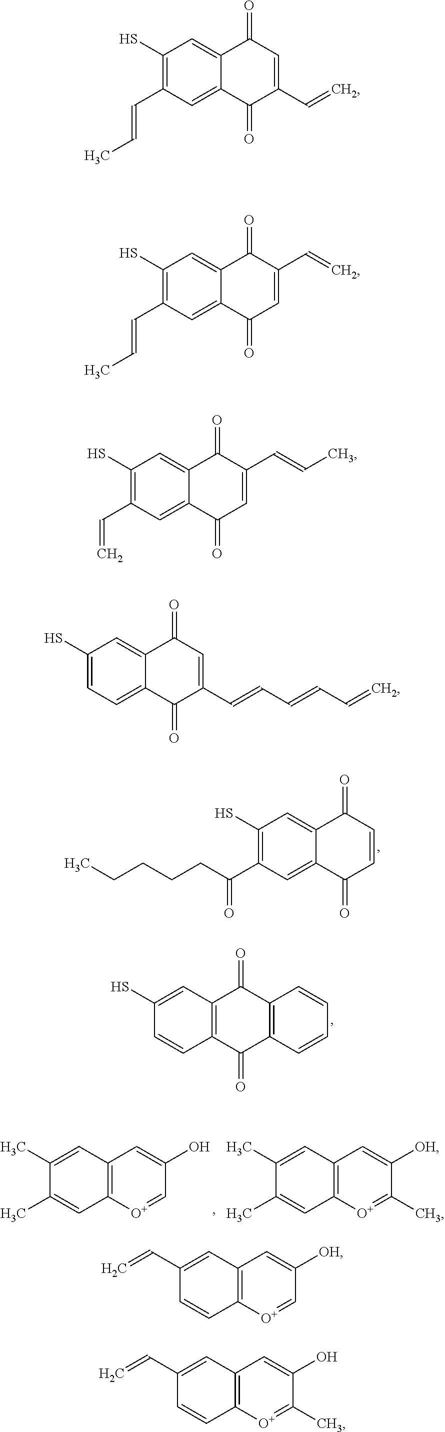

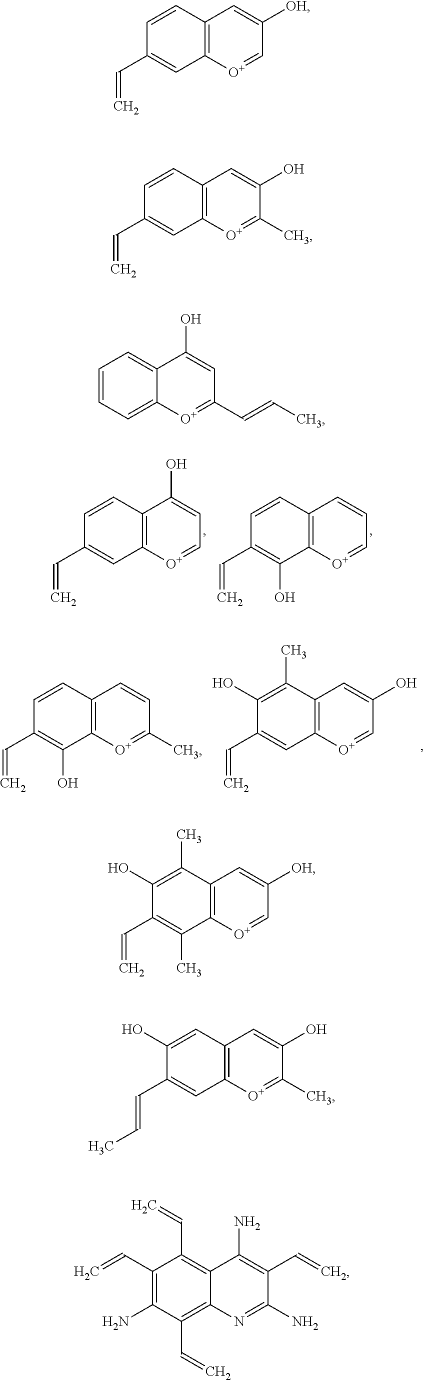

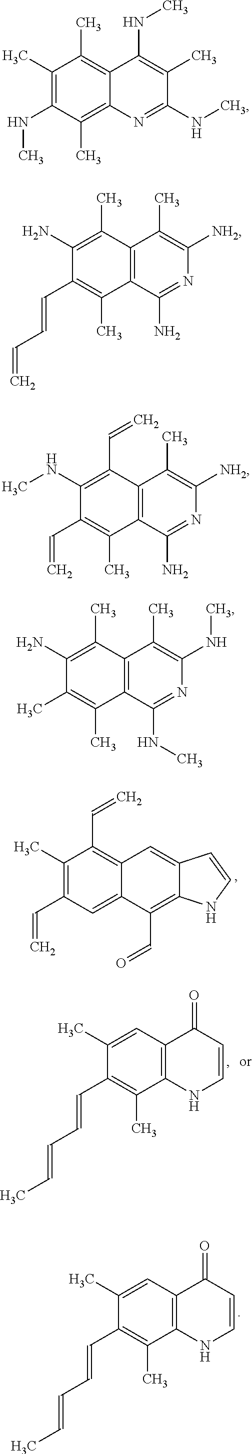

18. The method of claim 10, wherein the protonophore of Formula (I) is represented by a protonophore of Formula (IV) 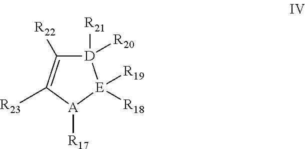
wherein:
A is O, S, C, or N;
D and E are each independently C or N;
R₁₇ is Absent, ═O, or Hydrogen;
R₁₈ is Absent, Hydrogen, CHO, OH, SH, CH₃, COCH₃, CH═CH₂, CH═CH(CH₃), CH═C(CH₃)₂, (CH═CH)₂CH₃, (CH═CH)₃CH₃, COCH═CH₂, COCH═CHCH₃, CO(CH₂)₃CH₃, CO(CH₂)₇CH₃, or CO-phenyl;
R₁₉ and R₂₀ are each independently Absent or Hydrogen;
R₂₀ is Absent, Hydrogen, OH, SH, CHO, COCH₃, CH₃, C(CH₃)₃, COCH═CH₂, COCH═CH(CH₃); CO(CH₂)₃CH₃, or CO(CH₂)₅CH₃; and
R₂₂ is Hydrogen, CH₃, C(CH₃)₃, CH═CH₂, CH═CH(CH₃), CH═C(CH₃)₂, CH═CHCH═CH₂, (CH═CH)₂CH₃, (CH═CH)₃CH₃, or COCH═CH₂, or a pharmaceutically acceptable salt, solvate, or prodrug thereof;
provided that: when A is O or S, then R₁₇ is Absent, when A is C, then R₁₇ is ═O, and when A is N, then R₁₇ is Hydrogen.

19. The method of claim 18, wherein the protonophore is
2-hydroxy, 3-acetyl, 4,5-di-(propen-1-yl), furan,
2-hydroxy, 3-(prop-2-en-1-one), 4,5-diethenyl, furan,
2,4-di-(prop-2-en-1-one), 3-hydroxy, 5-ethenyl, furan,
2-hydroxy, 3,5-diformyl, 4-[(1E)-buta-1,3-dien-1-yl], thiofuran,
2-hydroxy, 3-acetyl, 4-methyl, 5-ethenyl, thiofuran,
2-hydroxy, 3,5-acetyl, 4-[(1E,3E)-penta-1,3-dien-1-yl], thiofuran,
2-sulfanyl, 3-formyl, 4,5-di(propen-1-yl), furan,
2-sulfanyl, 3-(but-2-en-1-one), 4,5-diethenyl, furan,
2-sulfanyl, 3-(pentan-1-one), 4,5-dimethyl, furan,
2-methyl, 3-sulfanyl, 4-[(1E,3E,5E)-hepta-1,3,5-trien-1-yl], 5-formyl, furan,
2-methyl, 3-sulfanyl, 5-((2E,4E,6E)-octa-2,4,6-trien-1-one), furan,
3-sulfanyl, 5-(heptan-1-one), furan,
2,3-dithiol, 4-tert-butyl, 5-methyl, furan,
2,7-diacetyl, 3-hydroxy, 6-((1E,3E)-penta-1,3-dien-1-yl), benzofuran,
2-(prop-2-en-1-one), 3-hydroxy, 5-methyl, benzofuran,
2-acetyl, 3-hydroxy, 5-ethenyl, 6-methyl, benzofuran,
2-(but-2-en-1-one), 3-hydroxy, benzofuran,
2-acetyl, 3-hydroxy, 5-(propen-1-yl) benzofuran,
2,5-di-(prop-2-en-1-one), 3-hydroxy, benzofuran,
2-(but-2-en-1-one), 3-hydroxy, 5-acetyl, 6-methyl, benzofuran,
2-acetyl, 3-hydroxy, 5-(but-2-en-1-one), 6-methyl, benzofuran,
2-acetyl, 3-hydroxy, 5-(2-methylprop-1-en-1-yl), benzofuran,
2-acetyl, 3-hydroxy, 5,6-dimethyl, benzofuran,
2,6-di(propen-1-yl), 3-hydroxy, 5,7-diacetyl, benzofuran,
2-acetyl, 3-hydroxy, 7-(pentan-1-one), benzofuran,
2-(pentan-1-one), 3-hydroxy, 7-formyl, benzofuran,
2-formyl, 3-hydroxy, 5-(pentan-1-one), benzofuran,
2-(pentan-1-one), 3-hydroxy, 5-acetyl, benzofuran,
2,5-diethenyl, 3,7-dihydroxy, 4-formyl, 6-methyl, benzofuran,
2-(2-methylprop-1-en-1-yl), 3,7-dihydroxy, 4-acetyl, 6-ethenyl, benzofuran,
3,7-dihydroxy, 4-(but-2-en-1-one), 6-ethenyl, benzofuran,
2-(2-methylprop-1-en-1-yl), 3,7-dihydroxy, 6-acetyl, benzofuran,
2,4-diethenyl, 3,7-dihydroxy, 6-acetyl, benzofuran,
2-((1E,3E)-penta-1,3-dien-1-yl), 3-sulfanyl, 4,7-diformyl, benzofuran,
2-(propen-1-yl), 3-sulfanyl, 4,7-diformyl, 6-methyl, benzofuran,
2,5,6-trimethyl, 3-sulfanyl, 7-formyl, benzofuran,
2,7-dimethyl, 4,6-diformyl, 5-hydroxy, inden-1-one,
3,7-dimethyl, 4,6-diformyl, 5-hydroxy, inden-1-one,
4,6-diformyl, 5-hydroxy, 7-ethenyl, inden-1-one,
4-acetyl, 5-hydroxy, 6-(butan-1-one), inden-1-one,
4-formyl, 5-hydroxy, 6-(but-2-en-1-one), dihydro-inden-1-one,
4-acetyl, 5-hydroxy, 6-(but-2-en-1-one), dihydro-inden-1-one,
2-(propen-1-yl), 4-methyl, 5-sulfanyl, 6-formyl, inden-1-one,
2-(propen-1-yl), 3-methyl, 5-sulfanyl, inden-1-one,
2-ethenyl, 5-sulfanyl, 6-methyl, inden-1-one,
2,4-dimethyl, 5-sulfanyl, 6-(prop-2-en-1-one), inden-1-one,
2-((1E,3E,5E)-hepta-1,3,5-trien-1-yl), 4,6-diacetyl, 5-sulfanyl, inden-1-one,
4-acetyl, 5-sulfanyl, 6-(hexan-1-one), inden-1-one,
5-sulfanyl, 6-(pentan-1-one), inden-1-one,
4,7-diethenyl, 5-sulfanyl, 6-formyl, dihydro-inden-1-one,
5-sulfanyl, 6-(propen-1-yl), dihydro-inden-1-one,
4-formyl, 5-sulfanyl, 6-((1E,3E)-penta-1,3-dien-1-yl), dihydro-inden-1-one,
4-(pentan-1-one), 5-sulfanyl, dihydro-inden-1-one,
3,6-dihydroxy-9-oxofluorene-2,7-dicarbaldehyde,
3,6-dihydroxy-9-oxofluorene-4,5-dicarbaldehyde,
3,6-dihydroxy-9-oxofluorene-2,5-dicarbaldehyde,
2-tert-butyl, 4-(2-methyl-propen-1-yl), 5-hydroxy, oxazole,
2-(nonan-1-one), 4-methy, 5-hydroxy, oxazole,
2-benzoyl, 4-(2-methylprop-1-en-1-yl), 5-hydroxy, oxazole,
3-tert-butyl, 4-(propen-1-yl), 5-hydroxy, isoxazole,
3-(heptan-1-one), 4-methyl, 5-hydroxy, isoxazole, or
5,7-diethenyl-6-methyl-1H-naphtho[2,3-b]pyrrole-9-carbaldehyde.

20. The method of claim 18, wherein the protonophore is

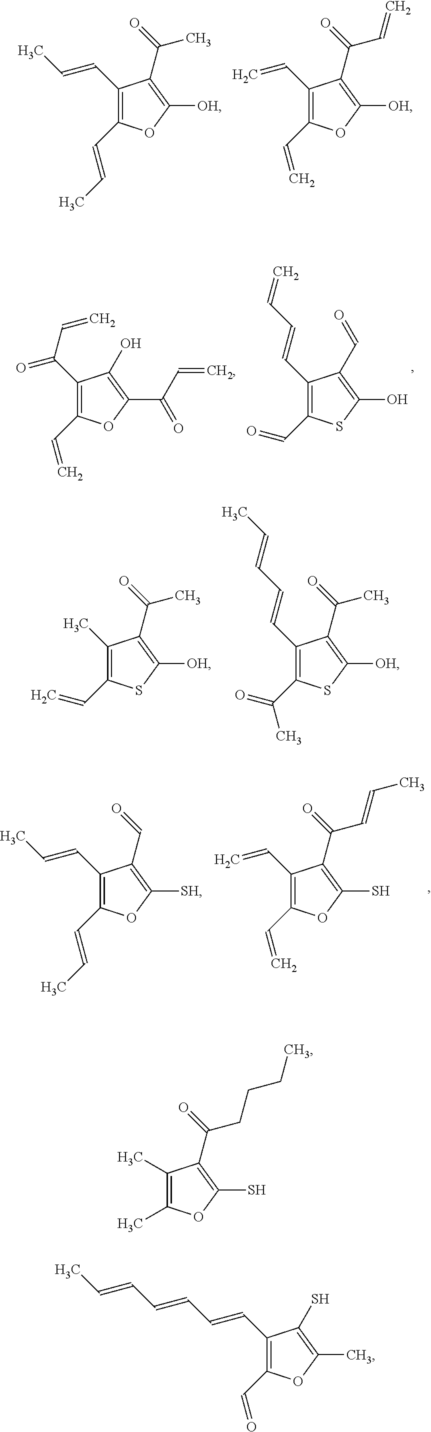

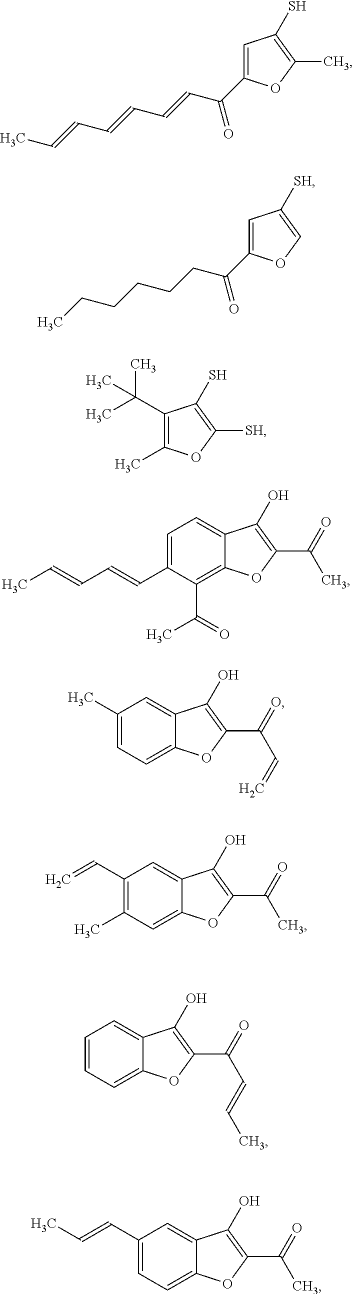

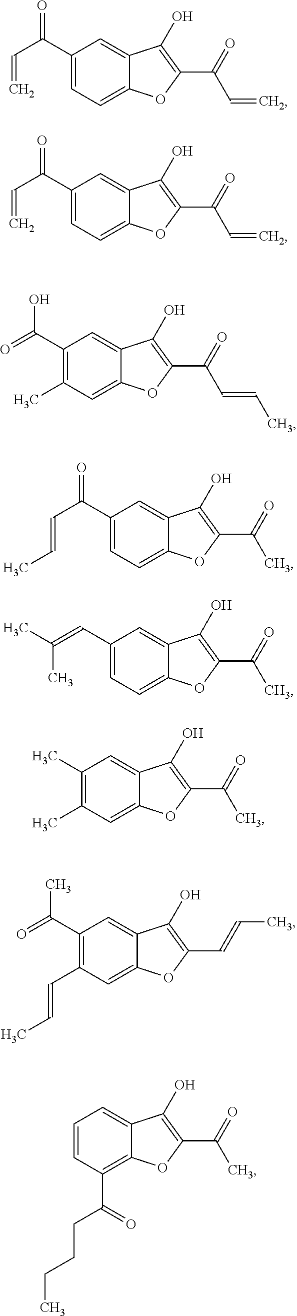

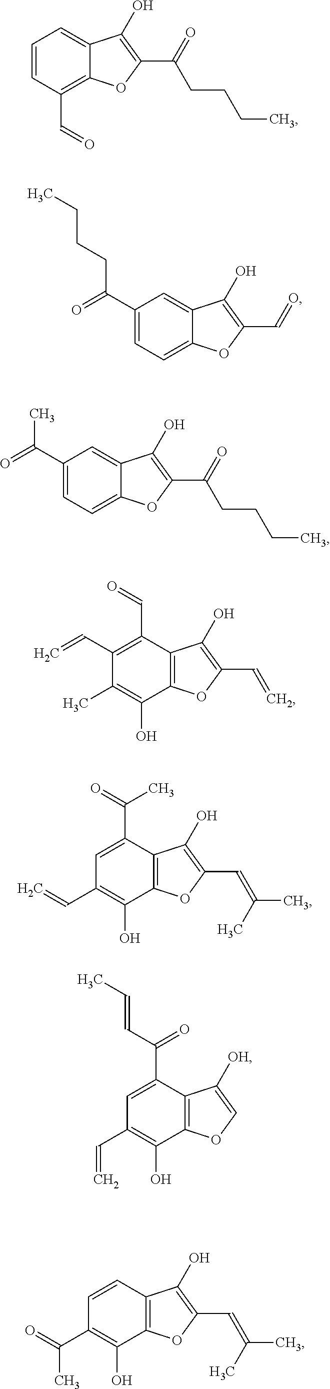

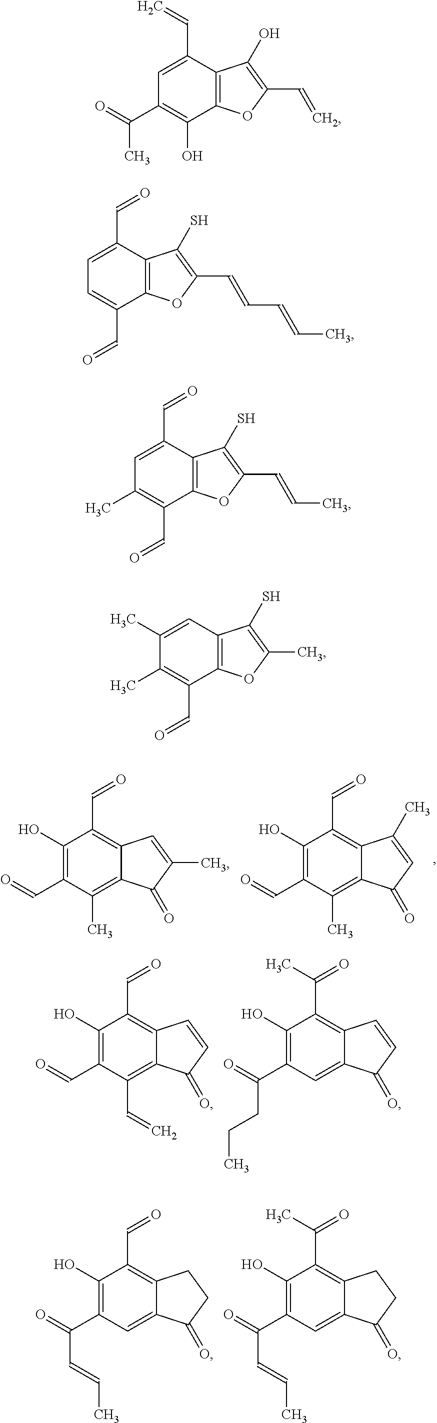

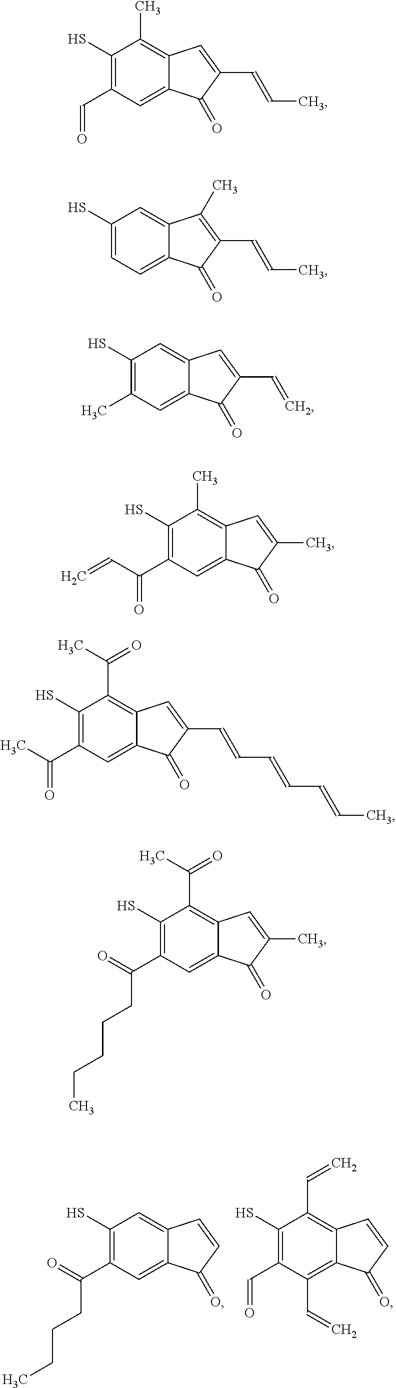

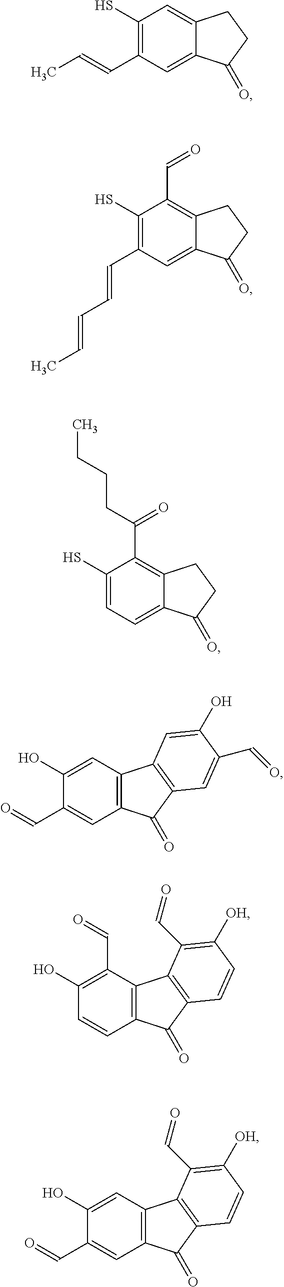

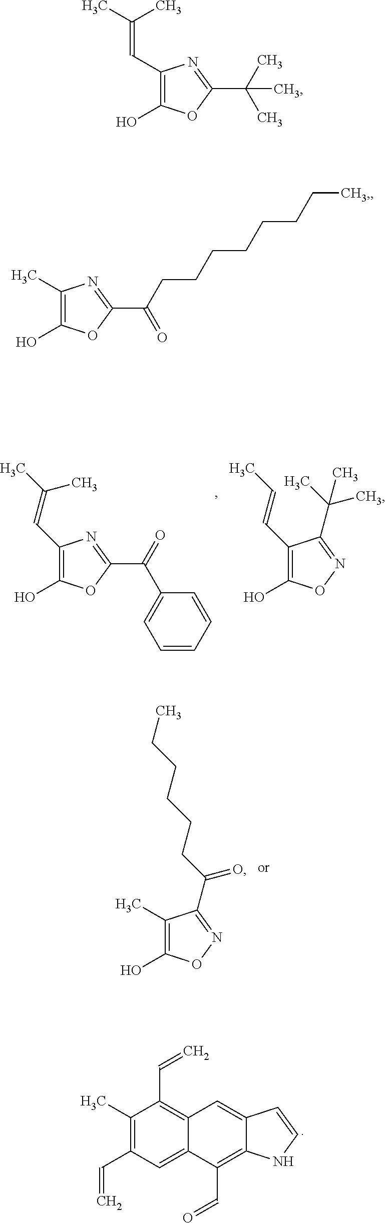

21. The method of claim 10, wherein the protonophore of Formula (I) is represented by a protonophore of Formula (V) 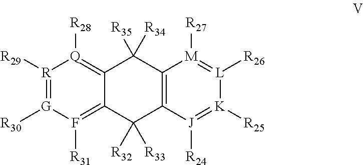
wherein:
G, K, L, Q, and R are each independently C;
F is O;
J. And M are each independently C, O;
R₂₄ and R₂₇ are each independently Absent or CH₃;
R₂₅ and R₂₆ are each independently CH₃ or OH;
R₂₈ and R₃₀ are each independently CH₃;
R₂₉ is OH;
R₃₁ is Absent; and
R₃₂, R₃₃, R₃₄, R₃₅ are each independently Hydrogen.

22. The method of claim 21, wherein the protonophore is
3,7-dihydroxy-2,4,6,8-tetramethyl-5H,10H-pyrano[3,2-g]chromene-1,9-bis(ylium) or
3,8-dihydroxy-2,7,9-tetramethyl-5H,10H-pyrano[2,3-g]chromene-1,6-bis(ylium).

23. The method of claim 21, wherein the protonophore is

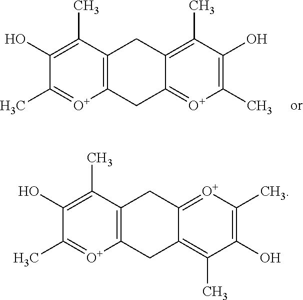 [SMILES: Cc1[o+]c2c(c(C)c1O)Cc1c([o+]c(C)c(O)c1C)C2 ; Cc1[o+]c2c(c(C)c1O)Cc1[o+]c(C)c(O)c(C)c1C2]

24. The pharmaceutical composition comprising a protonophore of Formula (II) 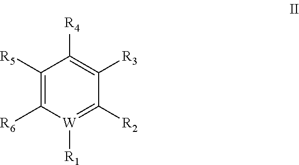
wherein:
W is C, O, S, or N;
R₁ is Absent, OH, or SH;
R₂ is Hydrogen, NH₂, CH3, C(CH₃)₃, COCH3, CHO, CH═CH₂, CH═CHCH₃, CH═CHCH═CH₂, (CH═CH)₂(CH₃), (CH═CH)₂CH═CH₂, or (CH═CH)₂CH═CH₂;
R₃ is Hydrogen, OH, SH, CH₃, CHO, CH═CH₂, CH═C(CH₃)₂, (CH═CH)₂(CH₃), or (CH═CH)₃(CH₃);
R₄ is CHO, CH₃, C(CH₃)₃, N(CH₃)₂, COCH₃, CH═CH₂, CH═CHCH₃, CH═CH₂CH═CH₂, (CH═CH)₂(CH₃), COCH═CH₂, CO(CH═CH)₂CH═CH₂, CO(CH═CH)₃(CH₃), or CO-p-C₆H₅SH;
R₅ is Hydrogen, CHO, CH₃, SH, COCH₃, C(CH₃)₃, CH═CH₂, CH═CHCH₃, CH═C(CH₃)₂ (CH═CH)₄(CH₃), or COCH═CH₂; and
R₆ is Hydrogen, NH₂, N(CH₃)₂, CHO, CH₃, OCH₃, COCH₃, CH═CH₂, CH═C(CH₃)₂, COCH═CH₂, or CO(CH₂)₇CH₃, or by a protonophore of Formula (III) 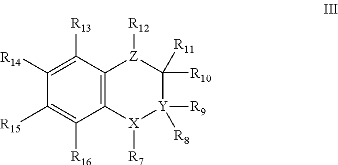
wherein:
X is O, C, or N;
Y is C or N;
Z is C;
R₇ is Absent, Hydrogen, CHO, ═O, NH₂, or NHCH₃, provided that when X is O, then R₇ is Absent, or when X is C, then R₇ is Hydrogen, CHO, ═O, NH₂, or NHCH₃ or when X is N, then R₇ is Absent or Hydrogen;
R₈ is Hydrogen, CH₃, NH₂, NHCH₃, COCH₃, CH═CH₂, CH═CHCH₃, CH═C(CH₃)₂, CH═CHCH═CH₂, (CH═CH)₂CH═CH₂, (CH═CH)₃CH₃, COCH═CH₂, COCH═CH(CH₃), COCH═C(CH₃)₂, or R₈, R₉, R₁₀ and R₁₁ together form the 3 and 4 carbon atoms of 2-carboxaldehyde-phenol, the 3 and 4 carbon atoms of 2-carboxaldehyde-6-methyl-phenol, the 3 and 4 carbon atoms of 2-acetyl-6-methyl-phenol, the 4 and 5 carbon atoms of 2-carboxaldehyde-phenol, the 3 and 4 carbon atoms of 2-carboxaldehyde-phenol, the 3 and 4 carbon atoms of 2-carboxaldehyde-6-methyl-phenol, the 3 and 4 carbon atoms of 2-carboxaldehyde-phenol, the 1 and 2 carbon atoms of phenyl, or the 2 and 3 carbon atoms of [2,3-b]1-H pyrole;
R₉ and R₁₁ are each independently Absent or Hydrogen;
R₁₀ is Hydrogen, OH, SH, NH₂, CH₃, NHCH₃, CH═CH₂, or CH═CH(CH₃);
R₁₂ is ═O, Hydrogen, NH₂, NHCH₃, or CH₃;
R₁₃ is Hydrogen, CHO, CH₃, COCH₃, or CH═CH₂;
R₁₄ is Hydrogen, OH, SH, NH₂, CH₃, CHO, COCH₃, NHCH₃, CH═CH₂, CH═CH(CH₃), C(CH₃)═CH(CH₃), CH═CHCH═CH₂, (CH═CH)₂CH═CH₂, (CH═CH)₃CH₃, COCH═CH₂, COCH═CHCH₃, CO(CH₂)₂CH₃, or CO(CH═CH)₂CH₃;
R₁₅ is Hydrogen, OH, CHO, SH, CH₃, NH₂, NHCH₃, COCH₃, CH═CH₂, CH═CHCH₃, CH═CHCH═CH₂, (CH═CH)₂(CH₃), COCH═CHCH₃, CO(CH₂)₃CH₃, or CO(CH₂)₄CH₃; and
R₁₆ is Hydrogen, CHO, CH₃, COCH₃, CH═CH₂, CH═CH(CH₃), (CH═CH)₂CH₃, (CH═CH)₂CH═CH₂, (CH═CH)₃CH₃, COCH═CH₂, COCH═CHCH₃, or CO(CH₂)₃CH₃, or by a protonophore of Formula (IV) 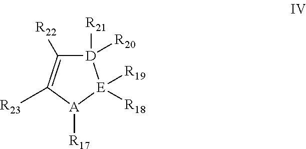
wherein:
A is O, S, C, or N;
D and E are each independently C or N;
R₁₇ is Absent, ═O, or Hydrogen;
R₁₈ is Absent, Hydrogen, CHO, OH, SH, CH₃, COCH₃, CH═CH₂, CH═CH(CH₃), CH═C(CH₃)₂, (CH═CH)₂CH₃, (CH═CH)₃CH₃, COCH═CH₂, COCH═CHCH₃, CO(CH₂)₃CH₃, CO(CH₂)₇CH₃, or CO-phenyl;
R₁₉ and R₂₀ are each independently Absent or Hydrogen;
R₂₀ is Absent, Hydrogen, OH, SH, CHO, COCH₃, CH₃, C(CH₃)₃, COCH═CH₂, COCH═CH(CH₃); CO(CH₂)₃CH₃, or CO(CH₂)₅CH₃; and
R₂₂ is Hydrogen, CH₃, C(CH₃)₃, CH═CH₂, CH═CH(CH₃), CH═C(CH₃)₂, CH═CHCH═CH₂, (CH═CH)₂CH₃, (CH═CH)₃CH₃, or COCH═CH₂; provided that: when A is O or S, then R₁₇ is Absent, when A is C, then R₁₇ is ═O, and when A is N, then R₁₇ is Hydrogen, or by a protonophore of Formula (V) 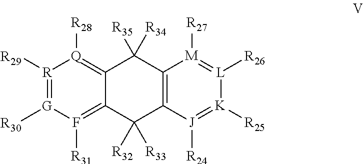
wherein:
G, K, L, Q, and R are each independently C;
F is O;
J. And M are each independently C, O;
R₂₄ and R₂₇ are each independently Absent or CH₃;
R₂₅ and R₂₆ are each independently CH₃ or OH;
R₂₈ and R₃₀ are each independently CH₃;
R₂₉ is OH;
R₃₁ is Absent;
R₃₂, R₃₃, R₃₄, R₃₅ are each independently Hydrogen, or a pharmaceutically acceptable salt, solvate, or prodrug thereof; and
a pharmaceutically acceptable diluent or carrier.

25. A method of inhibiting or killing a bacterium comprising contacting the bacterium with an effective anti-bacterial amount of a protonophore of Formula (II) 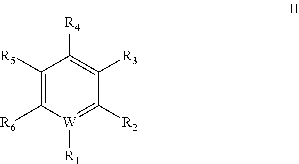
wherein:
W is C, O, S, or N;
R₁ is Absent, OH, or SH;
R₂ is Hydrogen, NH₂, CH₃, C(CH₃)₃, COCH3, CHO, CH═CH₂, CH═CHCH₃, CH═CHCH═CH₂, (CH═CH)₂(CH₃), (CH═CH)₂CH═CH₂, or (CH═CH)₂CH═CH₂;
R₃ is Hydrogen, OH, SH, CH₃, CHO, CH═CH₂, CH═C(CH₃)₂, (CH═CH)₂(CH₃), or (CH═CH)₃(CH₃);
R₄ is CHO, CH₃, C(CH₃)₃, N(CH₃)₂, COCH₃, CH═CH₂, CH═CHCH₃, CH═CH₂CH═CH₂, (CH═CH)₂(CH₃), COCH═CH₂, CO(CH═CH)₂CH═CH₂, CO(CH═CH)₃(CH₃), or CO-p-C₆H₅SH;
R₅ is Hydrogen, CHO, CH₃, SH, COCH₃, C(CH₃)₃, CH═CH₂, CH═CHCH₃, CH═C(CH₃)₂ (CH═CH)₄(CH₃), or COCH═CH₂; and
R₆ is Hydrogen, NH₂, N(CH₃)₂, CHO, CH₃, OCH₃, COCH₃, CH═CH₂, CH═C(CH₃)₂, COCH═CH₂, or CO(CH₂)₇CH₃; or
a protonophore of Formula (III) 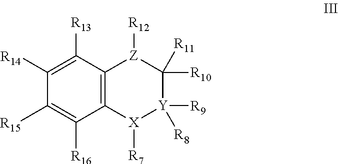
wherein:
X is O, C, or N;
Y is C or N;
Z is C;
R₇ is Absent, Hydrogen, CHO, ═O, NH₂, or NHCH₃, provided that when X is O, then R₇ is Absent, or when X is C, then R₇ is Hydrogen, CHO, ═O, NH₂, or NHCH₃ or when X is N, then R₇ is Absent or Hydrogen;
R₈ is Hydrogen, CH₃, NH₂, NHCH₃, COCH₃, CH═CH₂, CH═CHCH₃, CH═C(CH₃)₂, CH═CHCH═CH₂, (CH═CH)₂CH═CH₂, (CH═CH)₃CH₃, COCH═CH₂, COCH═CH(CH₃), COCH═C(CH₃)₂, or R₈, R₉, R₁₀ and R₁₁ together form the 3 and 4 carbon atoms of 2-carboxaldehyde-phenol, the 3 and 4 carbon atoms of 2-carboxaldehyde-6-methyl-phenol, the 3 and 4 carbon atoms of 2-acetyl-6-methyl-phenol, the 4 and 5 carbon atoms of 2-carboxaldehyde-phenol, the 3 and 4 carbon atoms of 2-carboxaldehyde-phenol, the 3 and 4 carbon atoms of 2-carboxaldehyde-6-methyl-phenol, the 3 and 4 carbon atoms of 2-carboxaldehyde-phenol, the 1 and 2 carbon atoms of phenyl, or the 2 and 3 carbon atoms of [2,3-b]1-H pyrole;
R₉ and R₁₁ are each independently Absent or Hydrogen;
R₁₀ is Hydrogen, OH, SH, NH₂, CH₃, NHCH₃, CH═CH₂, or CH═CH(CH₃);
R₁₂ is ═O, Hydrogen, NH₂, NHCH₃, or CH₃;
R₁₃ is Hydrogen, CHO, CH₃, COCH₃, or CH═CH₂;
R₁₄ is Hydrogen, OH, SH, NH₂, CH₃, CHO, COCH₃, NHCH₃, CH═CH₂, CH═CH(CH₃), C(CH₃)═CH(CH₃), CH═CHCH═CH₂, (CH═CH)₂CH═CH₂, (CH═CH)₃CH₃, COCH═CH₂, COCH═CHCH₃, CO(CH₂)2CH3, or CO(CH═CH)₂CH₃;
R₁₅ is Hydrogen, OH, CHO, SH, CH₃, NH₂, NHCH₃, COCH₃, CH═CH₂, CH═CHCH₃, CH═CHCH═CH₂, (CH═CH)₂(CH₃), COCH═CHCH₃, CO(CH₂)₃CH₃, or CO(CH₂)₄CH₃; and
R₁₆ is Hydrogen, CHO, CH₃, COCH₃, CH═CH₂, CH═CH(CH₃), (CH═CH)₂CH₃, (CH═CH)₂CH═CH₂, (CH═CH)₃CH₃, COCH═CH₂, COCH═CHCH₃, or CO(CH₂)₃CH₃; or
a protonophore of Formula (IV) 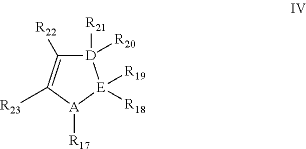
wherein:
A is O, S, C, or N;
D and E are each independently C or N;
R₁₇ is Absent, ═O, or Hydrogen;
R₁₈ is Absent, Hydrogen, CHO, OH, SH, CH₃, COCH₃, CH═CH₂, CH═CH(CH₃), CH═C(CH₃)₂, (CH═CH)₂CH₃, (CH═CH)₃CH₃, COCH═CH₂, COCH═CHCH₃, CO(CH₂)₃CH₃, CO(CH₂)₇CH₃, or CO-phenyl;
R₁₉ and R₂₀ are each independently Absent or Hydrogen;
R₂₀ is Absent, Hydrogen, OH, SH, CHO, COCH₃, CH₃, C(CH₃)₃, COCH═CH₂, COCH═CH(CH₃); CO(CH₂)₃CH₃, or CO(CH₂)₅CH₃; and
R₂₂ is Hydrogen, CH₃, C(CH₃)₃, CH═CH₂, CH═CH(CH₃), CH═C(CH₃)₂, CH═CHCH═CH₂, (CH═CH)₂CH₃, (CH═CH)₃CH₃, or COCH═CH₂; provided that: when A is O or S, then R₁₇ is Absent, when A is C, then R₁₇ is ═O, and when A is N, then R₁₇ is Hydrogen, or
a protonophore of Formula (V) 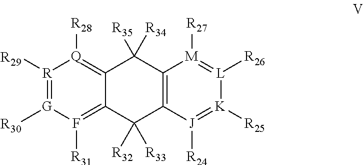
wherein:
G, K, L, Q, and R are each independently C;
F is O;
J and M are each independently C, O;
R₂₄ and R₂₇ are each independently Absent or CH₃;
R₂₅ and R₂₆ are each independently CH₃ or OH;
R₂₈ and R₃₀ are each independently CH₃;
R₂₉ is OH;
R₃₁ is Absent; and
R₃₂, R₃₃, R₃₄, R₃₅ are each independently Hydrogen, wherein the contacting is in vitro, in vivo, or directly on the bacterium.

26. A method of inhibiting or killing a fungus comprising contacting the fungus with an effective anti-fungal amount of a protonophore of Formula (II) 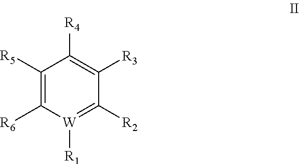
wherein:
W is C, O, S, or N;
R₁ is Absent, OH, or SH;
R₂ is Hydrogen, NH₂, CH3, C(CH₃)₃, COCH3, CHO, CH═CH₂, CH═CHCH₃, CH═CHCH═CH₂, (CH═CH)₂(CH₃), (CH═CH)₂CH═CH₂, or (CH═CH)₂CH═CH₂;
R₃ is Hydrogen, OH, SH, CH₃, CHO, CH═CH₂, CH═C(CH₃)₂, (CH═CH)₂(CH₃), or (CH═CH)₃(CH₃);
R₄ is CHO, CH₃, C(CH₃)₃, N(CH₃)₂, COCH₃, CH═CH₂, CH═CHCH₃, CH═CH₂CH═CH₂, (CH═CH)₂(CH₃), COCH═CH₂, CO(CH═CH)₂CH═CH₂, CO(CH═CH)₃(CH₃), or CO-p-C₆H₅SH;
R₅ is Hydrogen, CHO, CH₃, SH, COCH₃, C(CH₃)₃, CH═CH₂, CH═CHCH₃, CH═C(CH₃)₂ (CH═CH)₄(CH₃), or COCH═CH₂; and
R₆ is Hydrogen, NH₂, N(CH₃)₂, CHO, CH₃, OCH₃, COCH₃, CH═CH₂, CH═C(CH₃)₂, COCH═CH₂, or CO(CH₂)₇CH₃; or
a protonophore of Formula (III) 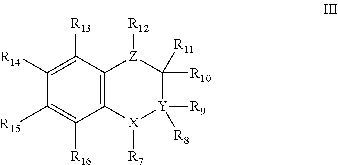
wherein:
X is O, C, or N;
Y is C or N;
Z is C;
R₇ is Absent, Hydrogen, CHO, ═O, NH₂, or NHCH₃, provided that when X is O, then R₇ is Absent, or when X is C, then R₇ is Hydrogen, CHO, ═O, NH₂, or NHCH₃ or when X is N, then R₇ is Absent or Hydrogen;
R₈ is Hydrogen, CH₃, NH₂, NHCH₃, COCH₃, CH═CH₂, CH═CHCH₃, CH═C(CH₃)₂, CH═CHCH═CH₂, (CH═CH)₂CH═CH₂, (CH═CH)₃CH₃, COCH═CH₂, COCH═CH(CH₃), COCH═C(CH₃)₂, or R₈, R₉, R₁₀ and R₁₁ together form the 3 and 4 carbon atoms of 2-carboxaldehyde-phenol, the 3 and 4 carbon atoms of 2-carboxaldehyde-6-methyl-phenol, the 3 and 4 carbon atoms of 2-acetyl-6-methyl-phenol, the 4 and 5 carbon atoms of 2-carboxaldehyde-phenol, the 3 and 4 carbon atoms of 2-carboxaldehyde-phenol, the 3 and 4 carbon atoms of 2-carboxaldehyde-6-methyl-phenol, the 3 and 4 carbon atoms of 2-carboxaldehyde-phenol, the 1 and 2 carbon atoms of phenyl, or the 2 and 3 carbon atoms of [2,3-b]1-H pyrole;
R₉ and R₁₁ are each independently Absent or Hydrogen;
R₁₀ is Hydrogen, OH, SH, NH₂, CH₃, NHCH₃, CH═CH₂, or CH═CH(CH₃);
R₁₂ is ═O, Hydrogen, NH₂, NHCH₃, or CH₃;
R₁₃ is Hydrogen, CHO, CH₃, COCH₃, or CH═CH₂;
R₁₄ is Hydrogen, OH, SH, NH₂, CH₃, CHO, COCH₃, NHCH₃, CH═CH₂, CH═CH(CH₃), C(CH₃)═CH(CH₃), CH═CHCH═CH₂, (CH═CH)₂CH═CH₂, (CH═CH)₃CH₃, COCH═CH₂, COCH═CHCH₃, CO(CH₂)2CH3, or CO(CH═CH)₂CH₃;
R₁₅ is Hydrogen, OH, CHO, SH, CH₃, NH₂, NHCH₃, COCH₃, CH═CH₂, CH═CHCH₃, CH═CHCH═CH₂, (CH═CH)₂(CH₃), COCH═CHCH₃, CO(CH₂)₃CH₃, or CO(CH₂)₄CH₃; and
R₁₆ is Hydrogen, CHO, CH₃, COCH₃, CH═CH₂, CH═CH(CH₃), (CH═CH)₂CH₃, (CH═CH)₂CH═CH₂, (CH═CH)₃CH₃, COCH═CH₂, COCH═CHCH₃, or CO(CH₂)₃CH₃, or
a protonophore of Formula (IV) 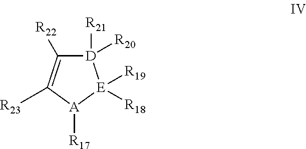
wherein:
A is O, S, C, or N;
D and E are each independently C or N;
R₁₇ is Absent, ═O, or Hydrogen;
R₁₈ is Absent, Hydrogen, CHO, OH, SH, CH₃, COCH₃, CH═CH₂, CH═CH(CH₃), CH═C(CH₃)₂, (CH═CH)₂CH₃, (CH═CH)₃CH₃, COCH═CH₂, COCH═CHCH₃, CO(CH₂)₃CH₃, CO(CH₂)₇CH₃, or CO-phenyl;
R₁₉ and R₂₀ are each independently Absent or Hydrogen;
R₂₀ is Absent, Hydrogen, OH, SH, CHO, COCH₃, CH₃, C(CH₃)₃, COCH═CH₂, COCH═CH(CH₃); CO(CH₂)₃CH₃, or CO(CH₂)₅CH₃; and
R₂₂ is Hydrogen, CH₃, C(CH₃)₃, CH═CH₂, CH═CH(CH₃), CH═C(CH₃)₂, CH═CHCH═CH₂, (CH═CH)₂CH₃, (CH═CH)₃CH₃, or COCH═CH₂; provided that: when A is O or S, then R₁₇ is Absent, when A is C, then R₁₇ is ═O, and when A is N, then R₁₇ is Hydrogen, or
a protonophore of Formula (V) 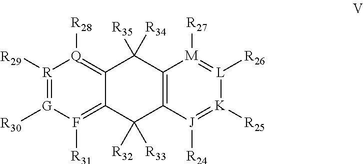
wherein:
G, K, L, Q, and R are each independently C;
F is O;
J and M are each independently C or O;
R₂₄ and R₂₇ are each independently absent or CH₃;
R₂₅ and R₂₆ are each independently CH₃ or OH;
R₂₈ and R₃₀ are each independently CH₃;
R₂₉ is OH;
R₃₁ is absent; and
R₃₂, R₃₃, R₃₄, R₃₅ are each independently Hydrogen, wherein the contacting is in vitro, in vivo, or directly on the fungus.

27. A method of inhibiting or killing a pest comprising contacting the pest with an effective pesticidal amount of a protonophore of Formula (II) 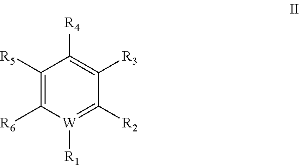
wherein:
W is C, O, S, or N;
R₁ is Absent, OH, or SH;
R₂ is Hydrogen, NH₂, CH3, C(CH₃)₃, COCH3, CHO, CH═CH₂, CH═CHCH₃, CH═CHCH═CH₂, (CH═CH)₂(CH₃), (CH═CH)₂CH═CH₂, or (CH═CH)₂CH═CH₂;
R₃ is Hydrogen, OH, SH, CH₃, CHO, CH═CH₂, CH═C(CH₃)₂, (CH═CH)₂(CH₃), or (CH═CH)₃(CH₃);
R₄ is CHO, CH₃, C(CH₃)₃, N(CH₃)₂, COCH₃, CH═CH₂, CH═CHCH₃, CH═CH₂CH═CH₂, (CH═CH)₂(CH₃), COCH═CH₂, CO(CH═CH)₂CH═CH₂, CO(CH═CH)₃(CH₃), or CO-p-C₆H₅SH;
R₅ is Hydrogen, CHO, CH₃, SH, COCH₃, C(CH₃)₃, CH═CH₂, CH═CHCH₃, CH═C(CH₃)₂ (CH═CH)₄(CH₃), or COCH═CH₂; and
R₆ is Hydrogen, NH₂, N(CH₃)₂, CHO, CH₃, OCH₃, COCH₃, CH═CH₂, CH═C(CH₃)₂, COCH═CH₂, or CO(CH₂)₇CH₃; or
a protonophore of Formula (III) 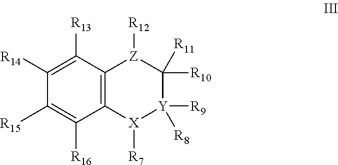
wherein:
X is O, C, or N;
Y is C or N;
Z is C;
R₇ is Absent, Hydrogen, CHO, ═O, NH₂, or NHCH₃, provided that when X is O, then R₇ is Absent, or when X is C, then R₇ is Hydrogen, CHO, ═O, NH₂, or NHCH₃ or when X is N, then R₇ is Absent or Hydrogen;
R₈ is Hydrogen, CH₃, NH₂, NHCH₃, COCH₃, CH═CH₂, CH═CHCH₃, CH═C(CH₃)₂, CH═CHCH═CH₂, (CH═CH)₂CH═CH₂, (CH═CH)₃CH₃, COCH═CH₂, COCH═CH(CH₃), COCH═C(CH₃)₂, or R₈, R₉, R₁₀ and R₁₁ together form the 3 and 4 carbon atoms of 2-carboxaldehyde-phenol, the 3 and 4 carbon atoms of 2-carboxaldehyde-6-methyl-phenol, the 3 and 4 carbon atoms of 2-acetyl-6-methyl-phenol, the 4 and 5 carbon atoms of 2-carboxaldehyde-phenol, the 3 and 4 carbon atoms of 2-carboxaldehyde-phenol, the 3 and 4 carbon atoms of 2-carboxaldehyde-6-methyl-phenol, the 3 and 4 carbon atoms of 2-carboxaldehyde-phenol, the 1 and 2 carbon atoms of phenyl, or the 2 and 3 carbon atoms of [2,3-b]1-H pyrole;
R₉ and R₁₁ are each independently Absent or Hydrogen;
R₁₀ is Hydrogen, OH, SH, NH₂, CH₃, NHCH₃, CH═CH₂, or CH═CH(CH₃);
R₁₂ is ═O, Hydrogen, NH₂, NHCH₃, or CH₃;
R₁₃ is Hydrogen, CHO, CH₃, COCH₃, or CH═CH₂;
R₁₄ is Hydrogen, OH, SH, NH₂, CH₃, CHO, COCH₃, NHCH₃, CH═CH₂, CH═CH(CH₃), C(CH₃)═CH(CH₃), CH═CHCH═CH₂, (CH═CH)₂CH═CH₂, (CH═CH)₃CH₃, COCH═CH₂, COCH═CHCH₃, CO(CH₂)2CH3, or CO(CH═CH)₂CH₃;
R₁₅ is Hydrogen, OH, CHO, SH, CH₃, NH₂, NHCH₃, COCH₃, CH═CH₂, CH═CHCH₃, CH═CHCH═CH₂, (CH═CH)₂(CH₃), COCH═CHCH₃, CO(CH₂)₃CH₃, or CO(CH₂)₄CH₃; and
R₁₆ is Hydrogen, CHO, CH₃, COCH₃, CH═CH₂, CH═CH(CH₃), (CH═CH)₂CH₃, (CH═CH)₂CH═CH₂, (CH═CH)₃CH₃, COCH═CH₂, COCH═CHCH₃, or CO(CH₂)₃CH₃, or
a protonophore of Formula (IV) 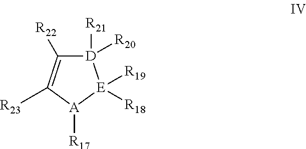
wherein:
A is O, S, C, or N;
D and E are each independently C or N;
R₁₇ is Absent, ═O, or Hydrogen;
R₁₈ is Absent, Hydrogen, CHO, OH, SH, CH₃, COCH₃, CH═CH₂, CH═CH(CH₃), CH═C(CH₃)₂, (CH═CH)₂CH₃, (CH═CH)₃CH₃, COCH═CH₂, COCH═CHCH₃, CO(CH₂)₃CH₃, CO(CH₂)₇CH₃, or CO-phenyl;
R₁₉ and R₂₀ are each independently Absent or Hydrogen;
R₂₀ is Absent, Hydrogen, OH, SH, CHO, COCH₃, CH₃, C(CH₃)₃, COCH═CH₂, COCH═CH(CH₃); CO(CH₂)₃CH₃, or CO(CH₂)₅CH₃; and
R₂₂ is Hydrogen, CH₃, C(CH₃)₃, CH═CH₂, CH═CH(CH₃), CH═C(CH₃)₂, CH═CHCH═CH₂, (CH═CH)₂CH₃, (CH═CH)₃CH₃, or COCH═CH₂; provided that: when A is O or S, then R₁₇ is Absent, when A is C, then R₁₇ is ═O, and when A is N, then R₁₇ is Hydrogen, or
a protonophore of Formula (V) 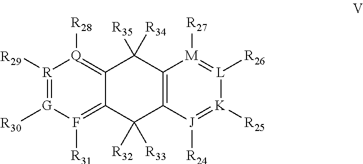
wherein:
G, K, L, Q, and R are each independently C;
F is O;
J and M are each independently C or O;
R₂₄ and R₂₇ are each independently Absent or CH₃;
R₂₅ and R₂₆ are each independently CH₃ or OH;
R₂₈ and R₃₀ are each independently CH₃;
R₂₉ is OH;
R₃₁ is Absent; and
R₃₂, R₃₃, R₃₄, R₃₅ are each independently Hydrogen, wherein the contacting is in vitro, in vivo, or directly on the pest.
